(19) United States
(12) Patent Application Publication
(10) Pub. No.: US 2026/0285855 A1
(43) Pub. Date: Sep. 24, 2026

(54) HETEROARYL DERIVATIVES AS DDRS INHIBITORS

(71) Applicant: CHIESI FARMACEUTICI S.P.A., Parma (IT)

(72) Inventors: Laura CARZANIGA, Parma (IT); Fabio RANCATI, Parma (IT); Jonathan Andrew SPENCER, Parma (IT); Benjamin Paul WHITTAKER, Parma (IT); Zuzana HAMASOVA, Parma (IT); Andrea RIZZI, Parma (IT); Roberta MAZZUCATO, Parma (IT); Stefano LEVANTO, Parma (IT); Robert Stuart Laurie CHAPMAN, Parma (IT); Gabriele FUMAGALLI, Parma (IT); Nicolò IOTTI, Parma (IT); Marta GIULIANI, Parma (IT); Dean Alfie STIMPSON, Parma (IT)

(73) Assignee: CHIESI FARMACEUTICI S.P.A., Parma (IT)

(21) Appl. No.: 19/490,007

(22) PCT Filed: Jun. 6, 2024
(86) PCT No.: PCT/EP2024/065550

§ 371 Date: Dec. 4, 2025

(30) Foreign Application Priority Data
Jun. 7, 2023 (EP) 23177905.9

(51) Int. Cl. C07D 471/04; A61K 31/4178; A61K 31/4188; A61K 31/429; A61K 31/4365; A61K 31/437; A61K 31/4427; A61K 31/4439; A61K 31/444; A61K 31/496; A61K 31/4985; A61K 31/501; A61K 31/5025; A61K 31/506; A61K 31/519; A61K 31/5377; A61K 31/5383; A61K 31/541; A61K 31/542; C07B 59/00; C07D 401/06; C07D 403/06; C07D 487/04; C07D 495/04; C07D 498/04; C07D 513/04

(52) U.S. Cl. CPC C07D 471/04; A61K 31/4178; A61K 31/4188; A61K 31/429; A61K 31/4365; A61K 31/437; A61K 31/4427; A61K 31/4439; A61K 31/444; A61K 31/496; A61K 31/4985; A61K 31/501; A61K 31/5025; A61K 31/506; A61K 31/519; A61K 31/5377; A61K 31/5383; A61K 31/541; A61K 31/542; C07B 59/002; C07D 401/06; C07D 403/06; C07D 487/04; C07D 495/04; C07D 498/04; C07D 513/04

(57) ABSTRACT

The present invention relates to compounds of formula (I) inhibiting Discoidin Domain Receptors (DDR inhibitors), methods of preparing such compounds, intermediate compounds useful in such preparations, pharmaceutical compositions containing them and therapeutic use thereof. The compounds of the invention may be useful for instance in the treatment of many disorders associated with DDR mechanisms.

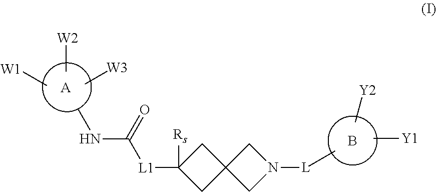

### FIELD OF THE INVENTION

The present invention relates to compounds inhibiting Discoidin Domain Receptors (DDR inhibitors), methods of preparing such compounds, intermediate compounds useful in such preparations, pharmaceutical compositions containing them and therapeutic use thereof.

The compounds of the invention may be useful for instance in the treatment of many disorders associated with DDR mechanisms.

### BACKGROUND OF THE INVENTION

Discoidin Domain Receptors (DDRs) are type I transmembrane receptor tyrosine kinase (RTKs). The DDR family comprises two distinct members, DDR1 and DDR2.

DDRs are unique receptors among the other members of the RTK superfamily, in that DDRs are activated by collagen whereas other members of the RTK superfamily are typically activated by soluble peptide-like growth factors (see Vogel, W. (1997) Mol. Cell 1, 13-23; Shrivastava A. Mol Cell. 1997; 1:25-34). Moreover, DDRs are unusual RTKs also because they form ligand-independent stable dimers that are non-covalently linked (see Noordeen, N. A. (2006) J. Biol. Chem. 281, 22744-22751; Mihai C. J Mol Biol. 2009; 385:432-445).

The DDR1 subfamily is composed of five membrane-anchored isoforms, and the DDR2 subfamily is represented by a single protein. The five DDR1 isoforms all have in common the extracellular and transmembrane domains but differ in the cytoplasmic region (see Valiathan, R. R. (2012) Cancer Metastasis Rev. 31, 295-321; Alves, F. (2001) FASEB J. 15, 1321-1323).

DDR receptor family has been found involved in a series of fibrotic diseases, such as pulmonary fibrosis, and in particular idiopathic pulmonary fibrosis (IPF), or kidney fibrosis. The first evidence for a protective role of DDR1 deletion in lung fibrosis was generated in 2006 by the research group of Dr. Vogel (see Avivi-Green C, Am J Respir Crit Care Med 2006; 174:420-427). The authors demonstrated that DDR1-null mice were largely protected against bleomycin (BLM)-induced injury. Furthermore, myofibroblast expansion and apoptosis were much lower in these animals compared with their wild-type counterparts. Absence of inflammation in knockout mice was confirmed by lavage cell count and cytokines ELISA. These results indicated that DDR1 expression is a prerequisite for the development of lung inflammation and fibrosis.

DDR2 deficiency or downregulation reduces bleomycin-induced lung fibrosis (see Zhao H, Bian H, Bu X, Zhang S, Zhang P, Yu J, et al Mol Ther 2016; 24:1734-1744). Zhao et al, demonstrated that DDR2 plays a critical role in the induction of fibrosis and angiogenesis in the lung, in particular that DDR2 synergizes with transforming growth factor (TGF)-β to induce myofibroblast differentiation. Furthermore, they showed that treatment of injured mice with specific siRNA against DDR2 exhibited therapeutic efficacy against lung fibrosis. In a second publication, Jia et al showed that mice lacking DDR2 are protected from bleomycin-induced lung fibrosis (see Jia S, Am J Respir Cell Mol Biol 2018; 59:295-305). In addition, DDR2-null fibroblasts are significantly more prone to apoptosis than wild-type fibroblasts, supporting a paradigm in which fibroblast resistance to apoptosis is critical for progression of fibrosis.

Discoidin Domain Receptor 1 (DDR1) is also a key factor of renal disease by promoting inflammation and fibrosis, and blocking the expression of DDR1 after the initiation of renal disease can delay or arrest the progression of this pathology (Kerroch et al., Sci Rep 6, 21262, 2016). DDR1 contributes to acute and chronic kidney injury by regulating BCR and STAT3 phosphorylation and in turn the production of MCP-1 and TGF-β. A DDR1-null mice model had reduced acute tubular injury, inflammation and tubulointerstitial fibrosis with overall decreased renal monocyte chemoattractant protein (MCP-1) levels and STAT3 activation. These findings identify DDR1 as an attractive therapeutic target for ameliorating both proinflammatory and profibrotic signaling in kidney disease (C. M. Borza et al., JCI Insight. 2022; 7 (3): e150887).

Some compounds have been described in the literature as DDR1 or DDR2 antagonists.

Expert Opin. Ther. Patents 2020, Vol. 30, No. 5, 341-350, by Guo Jing et al., is a review article of Discoidin Domain Receptor 1 (DDR1) modulators.

WO2023021278 and WO2023079291 disclose compounds as DDR1 and DDR2 inhibitors, useful in the treatment of cancer and fibrotic diseases, in particular in lung and kidney.

US 2018/148450 A1 discloses triaza-spiroenones as DDR1 inhibitors.

US 2019/374526 A1 discloses inhibitors of PR domain-containing protein 9. It discloses the conversion of 1-(3,5-dichlorophenyl)-3-(2-azaspiro[3.3]heptan-6-yl)urea HCl salt into 1-(3,5-dichlorophenyl)-3-(2-(2-methylpyridin-4-yl)-2-azaspiro[3.3]heptan-6-yl)urea (compound 239).

WO2023064370A1 discloses epoxide hydrolase (sEH) inhibitors. It discloses 1-(2-azaspiro[3.3]heptan-6-yl)-3-(4-(trifluoromethoxy)phenyl)urea at page 53, paragraph 219.

Of note, antagonizing the DDR receptors may be useful for the treatment of fibrosis and diseases, disorders or conditions that result from fibrosis. Even more, antagonizing both receptors DDR1 and DDR2 may be particularly efficacious in the treatment of the above-mentioned diseases, disorders or conditions.

Several efforts have been done in the past years to develop novel DDR1 and DDR2 receptor antagonists useful for the treatment of several diseases and some of those compounds have shown efficacy also in humans. However, there remains a potential for developing selective inhibitors of both receptors DDR1 and DDR2 useful for the treatment of diseases, disorders or conditions associated with a dysregulation of DDR receptors, in the respiratory field, in particular idiopathic pulmonary fibrosis (IPF), and in the field of fibrosis diseases in general, in particular kidney fibrosis.

New selective inhibitors of both receptors DDR1 and DDR2 could be administered by the inhalation route when characterized by a good inhalatory profile, corresponding to a good activity in the lung, a good lung retention and to a low metabolic stability in order to minimize the systemic exposure and correlated safety issues. Alternatively, new inhibitors could be administered by the oral route when characterized by a good oral profile, high oral bioavailability and low clearance with suitable physical-chemical properties.

In this direction, a new series of compounds of general formula (I), as herebelow reported, has been surprisingly found, which solves the problem of providing inhibitors for receptors DDR1 and DDR2 for administration by inhalation, which act as selective inhibitors of DDR1 and DDR2 receptors with respect to other human protein kinases. Such compounds show high potency, good inhalatory profile, low metabolic stability, low systemic exposure, improved safety and tolerability.

### SUMMARY OF THE INVENTION

In a first aspect the present invention relates to a compound of formula (I)

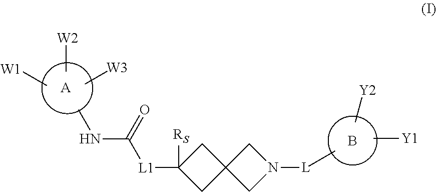

wherein
A is a ring selected from the group consisting of:

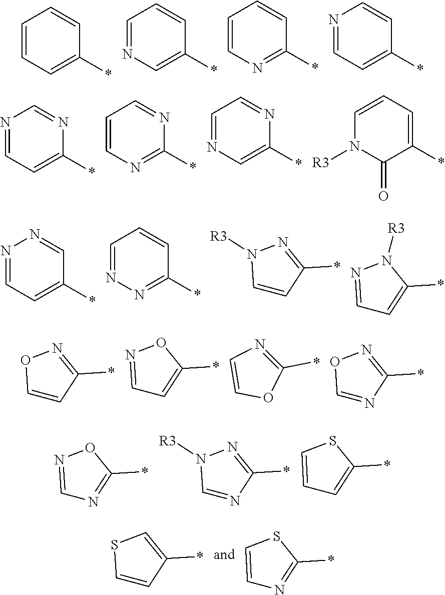

wherein [symbol] indicates a direct bond to NH;
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2—(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl, and are preferably selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Cl, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy, CH₂N(CH₃)₂ and 4-methylpiperazin-1-yl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms; and wherein preferably the 6-membered heterocycloalkyl is a pyranyl ring;
L is selected from CO and CH₂ or is absent;
L1 is selected from NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇)cycloalkyl-(C₁-C₄)alkyl and deuterated (C₁-C₄)alkyl, preferably selected from the group consisting of hydrogen, CD₃, cyclopropylmethyl, cyclopropyl, CH₂CF₃, CH₂CH₃ and CH₃;
R_{S} is selected from hydrogen and methyl, when L1 is NR;
Rs is hydrogen, when L1 is CH₂ or O;
B is mono- or bi-cyclic heteroaryl ring or bi-cyclic semisaturated heteroaryl ring, preferably selected from the group consisting of pyridinyl, pyrimidinyl, pyrazinyl, pyridazinyl, imidazolyl, pyrazolyl, pyrazolo[1,5-a]pyrazinyl, pyrazolo[1,5-a]pyridinyl, pyrazolo[3,4-d]pyrimidinyl, pyrazolo[1,5-a]pyrimidinyl, 1H-pyrazolo[3,4-b]pyridinyl, imidazo[1,2-b]pyridazinyl, imidazo[1,2-a]pyrazinyl, 1H-imidazo[1,2-b]pyrazolyl, imidazo[2,1-b]thiazolyl, pyrrolo[2,3-c]pyrimidinyl, pyrrolo[3,2-b]pyridinyl, 1H-pyrrolo[2,3-b]pyridinyl, pyrazolo[5,1-b]thiazolyl, imidazo[1,5-a]pyrazinyl, 5,6-dihydro-8H-imidazo[2,1-c][1,4]oxazinyl, thieno[3,2-d]pyrimidinyl, thieno[3,2-b]pyridinyl, thieno[2,3-d]pyrimidinyl, pyrazolo[5,1-b][1,3]thiazinyl, pyrrolo[3,2-d]pyrimidinyl, pyrrolo[2,3-d]pyrimidinyl and imidazo[1,2-a]pyridinyl;
Y1 and Y2 are substituents of ring B independently selected from the group consisting of hydrogen, (C₁-C₄)alkyl, deuterated (C₁-C₄)alkyl, (C₁-C₄)haloalkyl, (C₁-C₄)alkoxy, (C₁-C₄)haloalkoxy, (C₁-C₄)hydroxyalkyl, halogen, cyano, SF₅, (C₁-C₄) cyanoalkyl, (C₁-C₄)alkyl-sulfonyl, (C₁-C₄)haloalkyl-sulfonyl, CONR1R2, NHCOR1, NR1R2, NR1R2-(C₁-C₄)alkyl, heterocycloalkyl optionally substituted with 1 to 3 halogens, heterocycloalkyl-NH—(C₁-C₄)alkyl, (C₃-C₇)cycloalkyl, (C₁-C₄)alkyl-heterocycloalkyl, (C₁-C₄)alkyl-(C₃-C₇)cycloalkyl, heterocycloalkyl-(C₁-C₄)alkyl, (C₃-C₇)cycloalkyl-(C₁-C₄)alkyl, heterocycloalkyl-(C₁-C₄)alkoxy, (C₃-C₇)cycloalkyl-(C₁-C₄)alkoxy, heterocycloalkyl-oxy, (C₃-C₇)cycloalkyl-oxy, phenyl, (C₁-C₄)alkoxy substituted phenyl, (C₁-C₄)alkyl-thio-(C₁-C₄)alkyl, (C₁-C₄)alkyl-sulfonyl-(C₁-C₄)alkyl, (C₁-C₄)alkoxy-(C₁-C₄)alkyl, (C₁-C₄)haloalkyl-cycloalkyl, (C₁-C₄)alkyl-heterocycloalkyl-carbonyl and monocyclic heteroaryl, optionally substituted with 1 to 3 groups selected from the group consisting of (C₁-C₄)alkyl, (C₁-C₄)alkoxy and (C₁-C₄)alkoxy-(C₁-C₄)alkyl; wherein Y1 is preferably selected from the group consisting of hydrogen, CH₃, OCH₃, OCF₃, CF₃, C(CH₃)₃, C(CH₃)₂CF₃, CONH₂, OCF₂H, CF₂H, CF₂CH₃, CF₂CF₃, CH₂CF₃, CH₂OH, CH₂CH₂OH, CH₂CH₂OCH₃, CH₂CH₂SCH₃, CH₂CH₂F, CH₂SCH₃, SCF₃, SO₂CF₃, CD₃, cyano, NHCOCH₃, NH₂, (C₁-C₄)alkyl-piperazinyl, 4-methylpiperazin-1-yl, piperazinyl, morpholinyl, pyrrolidinyl, hydroxypyrrolidinyl, N-methyl-oxopiperazinyl, S,S-dioxido-thiomorpholinyl, oxetanyl-methyl, oxetanyl, (oxetanylamino)methyl, 1H-pyrazol-4-yl, oxazol-5-yl, pyridin-3-yl, pyrimidin-5-yl, pyridin-4-yl, dimethoxyphenyl, thiazol-5-yl, 3-isoxazol-5-yl and 1-methyl-1H-pyrazol-4-yl, and wherein Y2 is preferably hydrogen;
R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl; preferably, R1 and R2 are independently selected from the group consisting of hydrogen, (C₁-C₄)alkyl and optionally substituted heterocycloalkyl; more preferably, R1 and R2 are independently selected from the group consisting of hydrogen, methyl and oxetanyl;
R3 is selected from the group consisting of (C₁-C₄)alkyl, (C₁-C₄)haloalkyl, (C₁-C₄)alkyl-phenyl and monocyclic heteroaryl; preferably, R3 is CH₃ or p-tolyl;
or a stereoisomer, tautomer, solvate and pharmaceutically acceptable salt thereof;
wherein the compound of formula (I) is not 1-(3,5-dichlorophenyl)-3-(2-(2-methylpyridin-4-yl)-2-azaspiro[3.3]heptan-6-yl)urea.

In a second aspect, the invention refers to a pharmaceutical composition comprising a compound of formula (I), or pharmaceutically acceptable salts thereof, in admixture with at least one or more pharmaceutically acceptable carrier and/or excipient.

In a third aspect, the invention refers to a compound of formula (I), or pharmaceutically acceptable salts thereof, or to a pharmaceutical composition comprising a compound of formula (I), or pharmaceutically acceptable salts thereof, for use as a medicament.

In a further aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating a disease, disorder or condition associated with dysregulation of DDR.

In another aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating fibrosis and/or diseases, disorders or conditions that involve fibrosis.

In yet another aspect, the invention refers to a compound of formula (I), or pharmaceutically acceptable salts thereof, or to a pharmaceutical composition comprising a compound of formula (I), or pharmaceutically acceptable salts thereof, for use in preventing and/or treating idiopathic pulmonary fibrosis (IPF).

In a further aspect, the invention refers to processes for the preparation of compounds of formula (I) and to intermediate compounds that are useful in their preparation.

### DETAILED DESCRIPTION OF THE INVENTION

### Definitions

Unless otherwise specified, the compounds of formula (I) of the present invention are intended to include stereoisomers, tautomers, solvates and pharmaceutically acceptable salts thereof.

Unless otherwise specified, the compounds of formula (I) of the present invention are intended to include the compounds of formula (Ia), (Ib), (Ic), (Id) and (Ie).

The term “pharmaceutically acceptable salts”, as used herein, refers to derivatives of compounds of formula (I) wherein the parent compound is suitably modified by converting any of the free acid or basic groups, if present, into the corresponding addition salt with any base or acid conventionally intended as being pharmaceutically acceptable. Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals, such as potassium, sodium, calcium or magnesium.

The salts obtained by reacting the main compound, functioning as a base, with an inorganic or organic acid comprise, for example, salts of hydrochloric acid, hydrobromic acid, sulfuric acid, phosphoric acid, methanesulfonic acid, camphorsulfonic acid, acetic acid, oxalic acid, maleic acid, fumaric acid, succinic acid and citric acid.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “racemate” or “racemic mixture” refers to a composition composed of equimolar quantities of two enantiomeric species, wherein the composition is devoid of optical activity.

Wherein the compounds of formula (I) of the present invention comprise one or more stereogenic carbons, all stereoisomers thereof are included in the scope of the invention, both as racemic mixtures and as other stereoisomer mixtures in all proportions.

The term “halogen” or “halogen atoms” or “halo” as used herein includes fluorine (F), chlorine (Cl), bromine and iodine atom.

The term “(C₁-C₄)alkyl” refers to a straight or branched chain alkyl group having from 1 to 4 carbon atoms. Said term comprises methyl, ethyl, n-propyl, isopropyl, n-butyl, isobutyl, sec-butyl, and t-butyl. Such groups are herein indicated also by their atoms arrangement, i.e. as CH₃, CH₂CH₃, etc.

The term “(C₁-C₄)haloalkyl” refers to a straight or branched chain alkyl group having from 1 to 4 carbon atoms, comprising at least one halogen substituent. For example, the term comprises CF₃ and C(CH₃)₂CF₃.

The term “(C₁-C₄)alkoxy” refers to a straight or branched chain alkyl group having from 1 to 4 carbon atoms, comprising at least one oxygen atom, in particular, but not only, an oxygen atom directly linked to ring A or ring B, i.e. when W1, W2, W3, Y1 or Y2, respectively and independently, are (C₁-C₄)alkoxy. Examples include OCH₃ and OCH₂CH₃.

The term “(C₁-C₄)haloalkoxy” refers to a (C₁-C₄)alkoxy comprising at least one halogen substituent. Examples include OCF₃ and OCF₂H.

The term “(C₁-C₄)hydroxyalkyl” refers to a (C₁-C₄)alkyl comprising at least one hydroxy substituent. Examples include CH₂OH, CH(OH)CH₃ and CH₂CH₂OH.

The term “(C₃-C₇)cycloalkyl” refers to a saturated hydrocarbon ring comprising a number of ring carbon atoms from 3 to 7. Examples include cyclopropyl, cyclobutyl, cyclopentyl, cyclohexyl, and cycloheptyl.

The term “oxy” refers to an oxygen atom as the point of attachment to ring A or ring B of the substituent indicated right before “oxy”. For instance, “heterocycloalkyl-oxy” refers to a heterocycloalkyl substituent attached to ring A or ring B through an oxygen atom.

The term “deuterated” refers to a substituent wherein one or more hydrogen atoms are replaced with deuterium, like for instance CD₃.

The term “mono- or bi-cyclic heteroaryl (ring)” refers to a mono- or bi-cyclic aromatic group, comprising a number of ring atoms from 5 to 10 and comprising from 1 to 4, or 1 to 3, heteroatoms independently selected from N, S and O, and includes groups having two such monocyclic rings, or one such monocyclic ring and one monocyclic aryl ring, such as a phenyl ring, which are fused through a common bond or linked by a single bond. The mono- or bi-cyclic heteroaryl rings comprise pyrazolyl, furanyl, tiophenyl, oxazolyl, isoxazolyl, isothiazolyl, thiazolyl, imidazolyl, benzofuranyl, 1H-benzo[d]imidazolyl, 1H-indazolyl, benzothiophenyl, benzo[c]thiophenyl, quinazolinyl, pteridinyl, 1H-pyrazolo[5,1-c][1,2,4]triazolyl, pyrrolizinyl, indolizinyl, benzothiazolyl, pyrazolo[5,1-b]thiazolyl, 1H-imidazo[1,2-b]pyrazolyl, 1H-pyrazolo[3,4-b]pyridinyl, 1H-pyrazolo[3,4-d]pyrimidinyl, 1,6-dihydropyrrolo[2,3-b]pyrrolyl, 1,4-dihydropyrrolo[3,2-b]pyrrolyl, 4H-thieno[3,2-b]pyrrolyl, thieno[3,2-b]pyridinyl, isobenzofuranyl, 1,2,4-triazolyl, 1,2,5 oxadiazolyl, 1,2,3 oxadiazolyl, 1,2,5 thiadiazolyl, 1,2,3 thiadiazolyl, tetrazolyl, 6H-furo[2,3-b]pyrrolyl, 6H-thieno[2,3-b]pyrrolyl, 4H-furo[3,2-b]pyrrolyl, benzo[d]isothiazolyl, thiazolo[4,5-b]pyridinyl, 1,3,5-triazinyl, 1,2,3,4-thiatriazolyl, 1,2,3,4-oxatriazolyl, 1,2,3,4-tetrazinyl, 1,2,4,5-tetrazinyl, 1,2,3,5-tetrazinyl, 1H-imidazo[4,5-b]pyridinyl, 7H-purinyl, 1H-pyrrolyl, 1-methyl-1H-pyrrolyl, 1-methyl-1H-1,2,4-triazolyl, 1-methyl-1H-tetrazolyl, pyridinyl, pyrimidinyl, pyrazinyl, pyridazinyl, imidazo[1,2-b]pyridazinyl, pyrazolo[1,5-a]pyrazinyl, imidazo[1,2-a]pyrazinyl, imidazo[1,2-a]pyrazinyl, imidazo[1,2-a]pyridinyl, pyrazolo[1,5-a]pyridinyl, pyrazolo[1,5-a]pyrimidinyl, 1H-pyrrolo[2,3-b]pyridinyl, pyrrolo[2,3-d]pyrimidinyl, pyrrolo[3,2-d]pyrimidinyl, pyrrolo[2,3-c]pyridinyl, thieno[3,2-d]pyrimidinyl, imidazo[2,1-b]thiazolyl and imidazo[1,5-a]pyrazinyl.

The term “bi-cyclic semisaturated heteroaryl ring” refers to a bicyclic group, comprising a number of ring atoms from 7 to 11, and comprising from 1 to 4, or 1 to 3, heteroatoms independently selected from N, S and O, and comprising a mono-cyclic heteroaryl or phenyl ring condensed to a mono-cyclic heterocycloalkyl or (C₃-C₇)cycloalkyl. Examples of suitable bi-cyclic semisaturated heteroaryl ring include, for instance, 2,3-dihydro-1H-pyrrolo[2,3-b]pyridinyl, 6,7-dihydro-5H-pyrrolo[2,3-b]pyrazinyl, 4,5,6,7-tetrahydrooxazolo[4,5-c]pyridinyl, 4,5,6,7-tetrahydro-1H-pyrazolo[4,3-b]pyridinyl, 2,3,4,5-tetrahydro-1H-pyrrolo[2,3-b]pyrazinyl, 5,6-dihydro-8H-imidazo[2,1-c][1,4]oxazinyl, 6,7-dihydro-5H-pyrazolo[5,1-b][1,3]thiazinyl and 1-(4,5,6,7-tetrahydropyrazolo[1,5-a]pyridinyl).

The term “heterocycloalkyl” refers to a saturated or partly unsaturated mono-, bi- or spiro-cyclic ring system of 3 to 12 ring atoms comprising one or more, for instance 1 to 3, heteroatoms independently selected from N, S and O. This definition includes ring systems wherein S may be in different oxidation states, such as for instance S, SO or SO₂. This definition also includes ring systems wherein any ring carbon atom may be in different oxidation states, such as for instance CH₂, CH(OH) or CO. Examples of heterocycloalkyl include piperazinyl, pyrrolidinyl, azetidinyl, morpholinyl, S,S-dioxido-thiomorpholinyl, oxopiperazinyl and piperidinyl.

The term “spiro-cyclic ring system” refers to a saturated or partly unsaturated bi-cyclic ring system of 5 to 12 ring atoms, comprising one or more, for instance 1 to 3, heteroatoms selected from N, S and O, wherein the two rings have only one common carbon atom. Examples of spiro-cyclic ring systems include spiro[3.5]nonanyl, spiro[2.3]hexanyl, spiro[2.4]heptanyl, 2-azaspiro[3.3]heptanyl, 7l²-azaspiro[3.5]nonanyl and 2l²-azaspiro[3.5]nonanyl.

Any composite term, like for instance “(C₁-C₄)alkyl-heterocycloalkyl-carbonyl”, should be intended as conventionally construed by the groups from which it derives; in this example, it has to be construed by a (C₁-C₄)alkyl, a heterocycloalkyl and a carbonyl group which are linked together in the indicated sequence, and wherein the last group in the sequence, the carbonyl group in this example, is the point of attachment to the residual part of the compound of formula (I).

When referring to substituents, a dash (“-”) that is not between two letters, words, or symbols is meant to represent the point of attachment for such substituents.

The carbonyl group, when not indicated in words, is herein represented as CO.

Whenever basic amino groups are present in the compounds of formula (I), physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate. Likewise, in the presence of acidic groups, corresponding physiological cations may be present as well, for instance including alkaline or alkaline earth metal ions.

The term “Ki” indicates the dissociation constant for the enzyme-inhibitor complex, expressed in molar units. It is an indicator of the binding affinity between inhibitor and DDR1 or DDR2 receptors.

As above indicated, the present invention refers to a series of compounds represented by the general formula (I) as herein below described in detail, which are endowed with an inhibitory activity on receptors DDR1 and DDR2. Antagonizing receptors DDR1 and DDR2 can be particularly effective in the treatment of those diseases where the DDR receptors play a role, such as fibrosis and any other disease, disorder or condition related to fibrosis.

Indeed, as detailed in the experimental part below, the compounds of formula (I) of the present invention are able to act as inhibitors of both DDR1 and DDR2 receptors in a substantive and effective way. In particular, Table 44 further below shows that for representative compounds of the present invention the inhibitory activity against either DDR1 and/or DDR2 receptors, expressed as Ki, is lower than 100 nM in the binding assay. This confirms that the compounds of formula (I) are able to inhibit the two isoforms of DDR receptor mainly involved in fibrosis and diseases resulting from fibrosis. Accordingly, the compounds of formula (I) can be used in the treatment of fibrosis, in particular pulmonary fibrosis or kidney fibrosis, when DDR1 and DDR2 are involved.

As indicated in the experimental part, comparative compounds section, in particular in Table 7, conversely to compounds C1 and C2, characterized by having different linkers replacing the —NH—CO-L1-linker of the compounds of the invention, and conversely to compound C3, characterized by a flipped central core, the presence of said —NH—CO-L1-linker and of a central core as defined above in the compounds of the present invention unexpectedly and remarkably determines a high inhibitory activity on the DDR1 and DDR2 receptors.

Advantageously, the compounds of the present invention are endowed with a very high potency and could be administered in humans at very low dosages, thus potentially reducing the adverse events that typically may occur when administering drugs.

In addition to being notably potent with respect to their inhibitory activity on both receptors DDR1 and DDR2, the compounds of the present invention are also characterized by being selective inhibitors of DDR1 and DDR2 receptors with respect to other human protein kinases, and by a good inhalatory profile, that allows to act effectively on the lung compartment and have, at the same time, a low metabolic stability, that allows to minimize the drawbacks associated with systemic exposure, such as safety and tolerability issues.

Therefore, the compounds of the present invention may be particularly appreciated when looking at suitable and efficacious compounds useful for the treatment of fibrosis, in particular idiopathic pulmonary fibrosis, administered by the inhalation route and characterized by a good inhalatory profile, that corresponds to a good activity on the lung, a good lung retention and a low metabolic stability, that minimizes the systemic exposure and correlated safety issues.

Alternatively, the compounds of the present invention may be administered by the oral route for the treatment of fibrosis, when characterized by a good oral profile, high oral bioavailability and low clearance with suitable physical-chemical properties.

Accordingly, the present invention relates to a compound of formula (I)

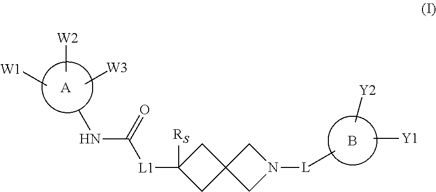

wherein
A is a ring selected from the group consisting of:

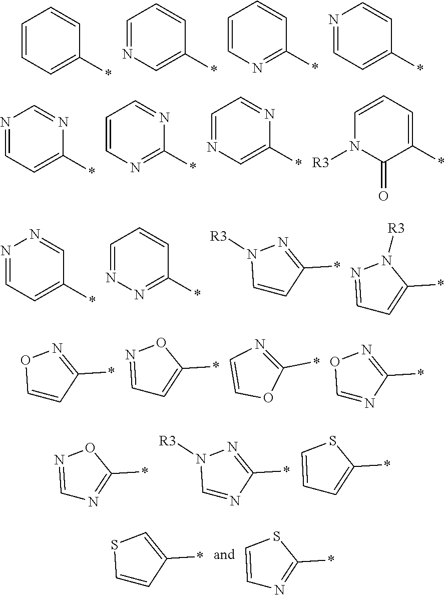

wherein [symbol] indicates a direct bond to NH;
   W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇) cycloalkyl-(C₁-C₄)alkoxy, heterocycloalkyl-oxy, (C₃-C₇) cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl, and are preferably selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Cl, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy, CH₂N(CH₃)₂ and 4-methylpiperazin-1-yl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 fluorine atoms; and wherein preferably the 6-membered heterocycloalkyl is a pyranyl ring;
   L is selected from CO and CH₂ or is absent;
   L1 is selected from NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇)cycloalkyl-(C₁-C₄)alkyl and deuterated (C₁-C₄)alkyl, preferably selected from the group consisting of hydrogen, CD₃, cyclopropylmethyl, CH₂CF₃, CH₂CH₃ and CH₃;
   Rs is selected from hydrogen and methyl, when L1 is NR;
   Rs is hydrogen when L1 is CH₂ or O;
   B is mono- or bi-cyclic heteroaryl ring or bi-cyclic semisaturated heteroaryl ring;
   Y1 and Y2 are substituents of ring B independently selected from the group consisting of hydrogen, (C₁-C₄)alkyl, deuterated (C₁-C₄)alkyl, (C₁-C₄)haloalkyl, (C₁-C₄)alkoxy, (C₁-C₄)haloalkoxy, (C₁-C₄)hydroxyalkyl, halogen, cyano, SF₅, (C₁-C₄) cyanoalkyl, (C₁-C₄)alkyl-sulfonyl, (C₁-C₄)haloalkyl-sulfonyl, CONR1R2, NHCOR1, NR1R2, NR1R2-(C₁-C₄)alkyl, heterocycloalkyl optionally substituted with 1 to 3 halogens, heterocycloalkyl-NH—(C₁-C₄)alkyl, (C₃-C₇)cycloalkyl, (C₁-C₄)alkyl-heterocycloalkyl, (C₁-C₄)alkyl-(C₃-C₇)cycloalkyl, heterocycloalkyl-(C₁-C₄)alkyl, (C₃-C₇)cycloalkyl-(C₁-C₄)alkyl, heterocycloalkyl-(C₁-C₄)alkoxy, (C₃-C₇)cycloalkyl-(C₁-C₄)alkoxy, heterocycloalkyl-oxy, (C₃-C₇)cycloalkyl-oxy, phenyl, (C₁-C₄)alkoxy substituted phenyl, (C₁-C₄)alkyl-thio-(C₁-C₄)alkyl, (C₁-C₄)alkyl-sulfonyl-(C₁-C₄)alkyl, (C₁-C₄)alkoxy-(C₁-C₄)alkyl, (C₁-C₄)haloalkyl-cycloalkyl, (C₁-C₄)alkyl-heterocycloalkyl-carbonyl and monocyclic heteroaryl, optionally substituted with 1 to 3 groups selected from the group consisting of (C₁-C₄)alkyl, (C₁-C₄)alkoxy and (C₁-C₄)alkoxy-(C₁-C₄)alkyl; wherein Y1 is preferably selected from the group consisting of hydrogen, CH₃, OCH₃, OCF₃, CF₃, C(CH₃)₃, C(CH₃)₂CF₃, CONH₂, OCF₂H, CF₂H, CF₂CH₃, CF₂CF₃, CH₂CF₃, CH₂OH, CH₂CH₂OH, CH₂CH₂OCH₃, CH₂CH₂SCH₃, CH₂CH₂F, CH₂SCH₃, SCF₃, SO₂CF₃, CD₃, cyano, NHCOCH₃, NH₂, (C₁-C₄)alkyl-piperazinyl, 4-methylpiperazin-1-yl, piperazinyl, morpholinyl, pyrrolidinyl, hydroxypyrrolidinyl, N-methyl-oxopiperazinyl, S,S-dioxido-thiomorpholinyl, oxetanyl-methyl, oxetanyl, (oxetanylamino)methyl, 1H-pyrazol-4-yl, oxazol-5-yl, pyridin-3-yl, pyrimidin-5-yl, pyridin-4-yl, dimethoxyphenyl, thiazol-5-yl, 3-isoxazol-5-yl and 1-methyl-1H-pyrazol-4-yl, and wherein Y2 is preferably hydrogen; in a preferred embodiment, Y1 is selected from the group consisting of hydrogen, CH₃ and 1-methyl-1H-pyrazol-4-yl, and Y2 is hydrogen; in another preferred embodiment, both Y1 and Y2 are hydrogen.
   R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl; optional substituents are preferably selected from the group consisting of CH₃, OCH₃, OCF₃, CF₃, C(CH₃)₃, C(CH₃)₂CF₃, CONH₂, OCF₂H and CF₂H.

R₃ is selected from the group consisting of (C₁-C₄)alkyl, (C₁-C₄)haloalkyl, (C₁-C₄)alkyl-phenyl and monocyclic heteroaryl; preferably, R3 is CH₃ or p-tolyl;
or a stereoisomer, tautomer, solvate and pharmaceutically acceptable salt thereof;
wherein the compound of formula (I) is not
1-(3,5-dichlorophenyl)-3-(2-(2-methylpyridin-4-yl)-2-azaspiro[3.3]heptan-6-yl)urea.

All the listed meanings of each of the variable moieties A, B, L, L1, R, W1, W2, W3, Y1, Y2, R1, R2, R3 and Rs of the compound of formula (I) of the invention have to be intended as alternatives and may be combined with each other in embodiments which are included in the scope of the invention.

Preferred halogens, as such and in groups comprising them, such as, for instance, (C₁-C₄)haloalkyl and (C₁-C₄)haloalkoxy substituents, are fluorine and chlorine, wherein fluorine is more preferred.

W1, W2 and W3 are substituents of ring A which can be attached to A at any available position. Ortho and meta are preferred positions.

In one embodiment, when A ring is phenyl and L1 is NH, W1, W2 and W3 are independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇) cycloalkyl, heterocycloalkyl-(C₁-C₄)alkyl, heterocycloalkyl-NH—(C₁-C₄)alkyl, (C₃-C₇) cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₃-C₆)cycloalkyl or the 5- or 6-membered heterocycloalkyl are optionally substituted with 1 to 3 halogen atoms; and wherein preferably the 6-membered heterocycloalkyl is a pyranyl ring; W1, W2 and W3 are preferably selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy, CH₂N(CH₃)₂, and 4-methylpiperazin-1-yl; more preferably, W1, W2 and W3 are selected from the group consisting of H, CH₃, OCH₃, OCF₃, CF₃, OCF₂H, cyano, SF₅, F, Br, I, CH₂OH, CF₂CF₃ and CH₂CF₃.

In another embodiment, W1, W2 and W3 are independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇) cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇) cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms; and wherein preferably the 6-membered heterocycloalkyl is a pyranyl ring; W1, W2 and W3 are preferably selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy, CH₂N(CH₃)₂, and 4-methylpiperazin-1-yl; more preferably, W1, W2 and W3 are selected from the group consisting of H, CH₃, OCH₃, OCF₃, CF₃, OCF₂H, cyano, SF₅, F, Br, I, CH₂OH, CF₂CF₃ and CH₂CF₃.

L is a linker and may be selected from CO and CH₂ or is absent.

L1 is a linker and may be selected from NR, CH₂ and O, wherein R is preferably selected from the group consisting of hydrogen, CD₃, cyclopropylmethyl, cyclopropyl, CH₂CF₃, CH₂CH₃ and CH₃.

Rs is hydrogen or methyl when L1 is NR; Rs is hydrogen when L1 is CH₂ or O.

B is preferably selected from the group consisting of pyridinyl, pyrimidinyl, pyrazinyl, pyridazinyl, imidazolyl, pyrazolyl, pyrazolo[1,5-a]pyrazinyl, pyrazolo[1,5-a]pyridinyl, pyrazolo[3,4-d]pyrimidinyl, pyrazolo[1,5-a]pyrimidinyl, 1H-pyrazolo[3,4-b]pyridinyl, imidazo[1,2-b]pyridazinyl, imidazo[1,2-a]pyrazinyl, 1H-imidazo[1,2-b]pyrazolyl, imidazo[2,1-b]thiazolyl, pyrrolo[2,3-c]pyrimidinyl, pyrrolo[3,2-b]pyridinyl, 1H-pyrrolo[2,3-b]pyridinyl, pyrazolo[5,1-b]thiazolyl, imidazo[1,5-a]pyrazinyl, 5,6-dihydro-8H-imidazo[2,1-c][1,4]oxazinyl, thieno[3,2-d]pyrimidinyl, thieno[3,2-b]pyridinyl, thieno[2,3-d]pyrimidinyl, pyrazolo[5,1-b][1,3]thiazinyl, pyrrolo[3,2-d]pyrimidinyl, pyrrolo[2,3-d]pyrimidinyl and imidazo[1,2-a]pyridinyl. Any ring B may be attached to the L moiety at any available position of its mono- or bi-cyclic ring system.

Y1 is a substituent of ring B which can be attached to B at any available position. Y1 is preferably selected from the group consisting of hydrogen, CH₃, OCH₃, OCF₃, CF₃, C(CH₃)₃, C(CH₃)₂CF₃, CONH₂, OCF₂H, CF₂H, CF₂CH₃, CF₂CF₃, CH₂CF₃, CH₂OH, CH₂CH₂OH, CH₂CH₂OCH₃, CH₂CH₂SCH₃, CH₂CH₂F, CH₂SCH₃, SCF₃, SO₂CF₃, CD₃, cyano, NHCOCH₃, NH₂, (C₁-C₄)alkyl-piperazinyl, 4-methylpiperazin-1-yl, piperazinyl, morpholinyl, pyrrolidinyl, hydroxypyrrolidinyl, N-methyl-oxopiperazinyl, S,S-dioxido-thiomorpholinyl, oxetanyl-methyl, oxetanyl, (oxetanylamino)methyl, 1H-pyrazol-4-yl, oxazol-5-yl, pyridin-3-yl, pyrimidin-5-yl, pyridin-4-yl, dimethoxyphenyl, thiazol-5-yl, 3-isoxazol-5-yl and 1-methyl-1H-pyrazol-4-yl; more preferably, Y1 is selected from the group consisting of hydrogen, CH₃ and 1-methyl-1H-pyrazol-4-yl.

Y2 is a substituent of ring B which can be attached to B at any available position. Y2 may have the same preferred meanings as Y1, independently. In more preferred embodiments Y2 is hydrogen.

R1 and R2 are preferably independently selected from the group consisting of hydrogen, (C₁-C₄)alkyl and optionally substituted heterocycloalkyl; more preferably, R1 and R2 are independently selected from the group consisting of hydrogen, methyl and oxetanyl.

R3 is selected from the group consisting of (C₁-C₄)alkyl, (C₁-C₄)haloalkyl, (C₁-C₄)alkyl-phenyl and monocyclic heteroaryl, being preferably CH₃ or p-tolyl.

Accordingly, in a preferred embodiment, the invention relates to a compound of formula (I) wherein
A is a ring selected from the group consisting of:

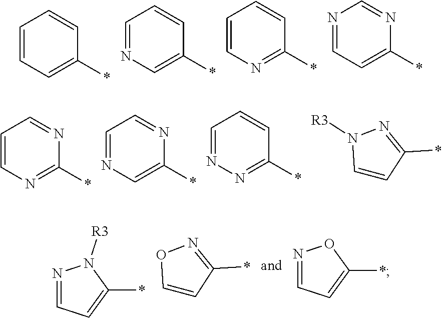

even more preferably, A is selected from the group consisting of phenyl, 3-pyridinyl, 2-pyridinyl, 3-pyridazinyl, 3-isoxazolyl and pyrazolyl, wherein R3 is CH₃ or p-tolyl;
W1, W2 and W3 are selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy and CH₂N(CH₃)₂; more preferably, W1, W2 and W3 are selected from the group consisting of H, CH₃, OCH₃, OCF₃, CF₃, OCF₂H, cyano, SF₅, F, Br, I, CH₂OH, CF₂CF₃ and CH₂CF₃.
L is selected from CO and CH₂ or is absent, being preferably CO;
L1 is selected from NR, CH₂ and O, being preferably NR, wherein R is preferably selected from the group consisting of hydrogen, CD₃, cyclopropylmethyl, cyclopropyl, CH₂CF₃, CH₂CH₃ and CH₃;
B is selected from the group consisting of pyridinyl, pyrimidinyl, pyrazinyl, pyridazinyl, imidazolyl, pyrazolyl, pyrazolo[1,5-a]pyrazinyl, pyrazolo[1,5-a]pyridinyl, pyrazolo[3,4-d]pyrimidinyl, pyrazolo[1,5-a]pyrimidinyl, 1H-pyrazolo[3,4-b]pyridinyl, imidazo[1,2-b]pyridazinyl, imidazo[1,2-a]pyrazinyl, 1H-imidazo[1,2-b]pyrazolyl, imidazo[2,1-b]thiazolyl, pyrrolo[2,3-c]pyrimidinyl, pyrrolo[3,2-b]pyridinyl, 1H-pyrrolo[2,3-b]pyridinyl, pyrazolo[5,1-b]thiazolyl, imidazo[1,5-a]pyrazinyl, 5,6-dihydro-8H-imidazo[2,1-c][1,4]oxazinyl, thieno[3,2-d]pyrimidinyl, thieno[3,2-b]pyridinyl, thieno[2,3-d]pyrimidinyl, pyrazolo[5,1-b][1,3]thiazinyl, pyrrolo[3,2-d]pyrimidinyl, pyrrolo[2,3-d]pyrimidinyl and imidazo[1,2-a]pyridinyl;
Y1 is selected from the group consisting of hydrogen, CH₃, OCH₃, OCF₃, CF₃, C(CH₃)₃, C(CH₃)₂CF₃, CONH₂, OCF₂H, CF₂H, CF₂CH₃, CF₂CF₃, CH₂CF₃, CH₂CH₂OH, CH₂CH₂OCH₃, CH₂CH₂SCH₃, CH₂CH₂F, CH₂SCH₃, SCF₃, SO₂CF₃, CD₃, cyano, NHCOCH₃, NH₂, (C₁-C₄)alkyl-piperazinyl, 4-methylpiperazin-1-yl, piperazinyl, morpholinyl, pyrrolidinyl, hydroxypyrrolidinyl, N-methyl-oxopiperazinyl, S,S-dioxido-thiomorpholinyl, oxetanyl-methyl, oxetanyl, (oxetanylamino)methyl, 1H-pyrazol-4-yl, oxazol-5-yl, pyridin-3-yl, pyrimidin-5-yl, pyridin-4-yl, dimethoxyphenyl, thiazol-5-yl, 3-isoxazol-5-yl and 1-methyl-1H-pyrazol-4-yl, and Y2 is hydrogen; and
R1 and R2 are preferably independently selected from the group consisting of hydrogen, (C₁-C₄)alkyl and optionally substituted heterocycloalkyl; more preferably, R1 and R2 are independently selected from the group consisting of hydrogen, methyl and optionally substituted oxetanyl, wherein optional substituents, when present, are selected from the group consisting of CH₃, OCH₃, OCF₃, CF₃, C(CH₃)₃, C(CH₃)₂CF₃, CONH₂, OCF₂H and CF₂H;
or a stereoisomer, tautomer, solvate and pharmaceutically acceptable salt thereof.

In another preferred embodiment, the present invention relates to a compound of formula (I) wherein L is CO and L1 is NR, which is represented by formula (Ia)

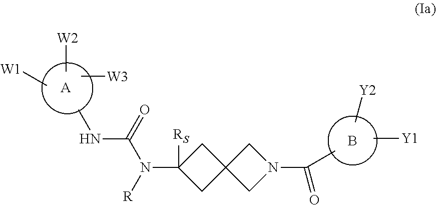

wherein W1, W2, W3, A, B, R, Rs, Y1 and Y2 are as defined above.

In yet another preferred embodiment, the present invention relates to a compound of formula (I), wherein L is CH₂ and L1 is NR, which is represented by formula (Ib)

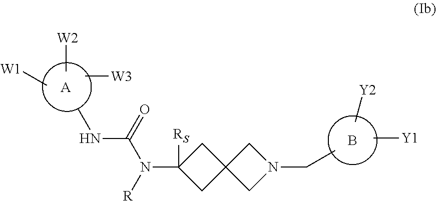

wherein W1, W2, W3, A, B, R, Rs, Y1 and Y2 are as defined above.

In a further preferred embodiment, the present invention relates to a compound of formula (I), wherein L is absent and L1 is NR, which is represented by formula (Ic)

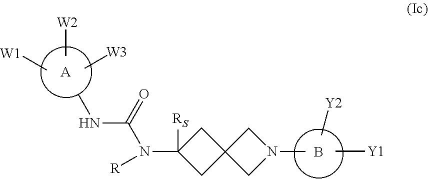

wherein W1, W2, W3, A, B, R, Rs, Y1 and Y2 are as defined above.

In another preferred embodiment, the present invention relates to a compound of formula (I), wherein L1 is CH₂, which is represented by formula (Id)

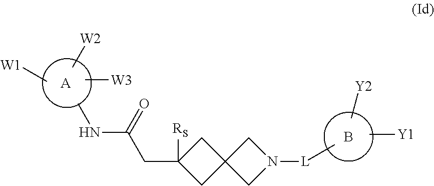

wherein W1, W2, W3, A, B, L, Y1 and Y2 are as defined above and Rs is hydrogen. In a particularly preferred embodiment, the present invention relates to a compound of formula (Id) wherein L is CO.

In another preferred embodiment, the present invention relates to a compound of formula (I), wherein L1 is O, which is represented by formula (Ie)

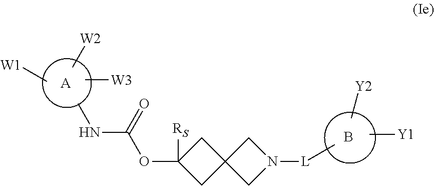

wherein W1, W2, W3, A, B, L, Y1 and Y2 are as defined above and Rs is hydrogen. In a particularly preferred embodiment, the present invention relates to a compound of formula (Ie) wherein L is CO.

Particularly preferred embodiments of the invention are the compounds of formula (Ia) listed in Table 1 below, and pharmaceutically acceptable salts thereof. These compounds are particularly active on receptors DDR1 and DDR2, as shown in Table 44 further below.

TABLE 1
List of representative compounds of Formula (Ia)

| Example No | Structure | Chemical Name |
|---|---|---|
| 1 | 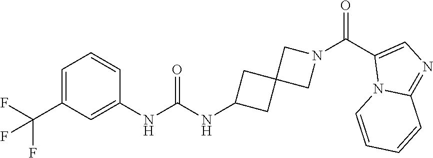 [SMILES: O=C(Nc1cccc(C(F)(F)F)c1)NC1CC2(C1)CN(C(=O)c1cnc3ccccn13)C2] | 1-(2-(imidazo[1,2-a]pyridine- 3-carbonyl)-2- azaspiro[3.3]heptan-6-yl)-3-(3- (trifluoromethyl)phenyl)urea |
| 2 | 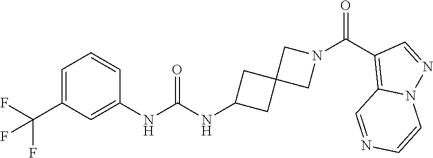 [SMILES: O=C(Nc1cccc(C(F)(F)F)c1)NC1CC2(C1)CN(C(=O)c1cnn3ccncc13)C2] | 1-(2-(pyrazolo[1,5-a]pyrazine- 3-carbonyl)-2- azaspiro[3.3]heptan-6-yl)-3-(3- (trifluoromethyl)phenyl)urea |
| 3 | 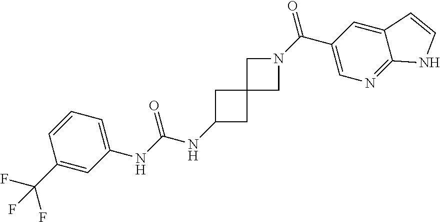 [SMILES: O=C(Nc1cccc(C(F)(F)F)c1)NC1CC2(C1)CN(C(=O)c1cnc3[nH]ccc3c1)C2] | 1-(2-(1H-pyrrolo[2,3- b]pyridine-5-carbonyl)-2- azaspiro[3.3]heptan-6-yl)-3-(3- (trifluoromethyl)phenyl)urea |
| 4 | 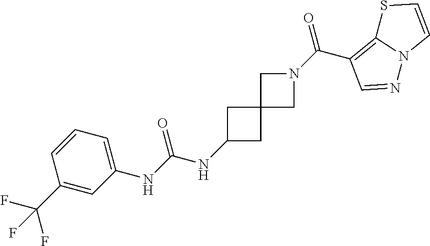 [SMILES: O=C(Nc1cccc(C(F)(F)F)c1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(3- (trifluoromethyl)phenyl)urea |
| 5 | 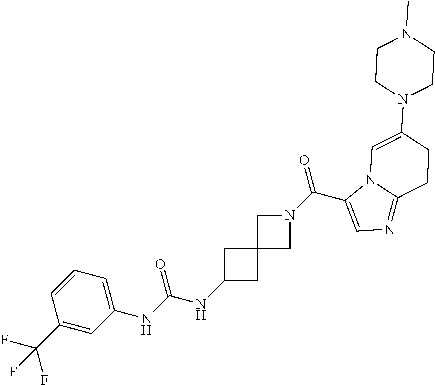 [SMILES: CN1CCN(C2=Cn3c(C(=O)N4CC5(CC(NC(=O)Nc6cccc(C(F)(F)F)c6)C5)C4)cnc3CC2)CC1] | 1-(2-(6-(4-methylpiperazin-1- yl)imidazo[1,2-a]pyridine-3- carbonyl)-2- azaspiro[3.3]heptan-6-yl)-3-(3- (trifluoromethyl)phenyl)urea |
| 6 | 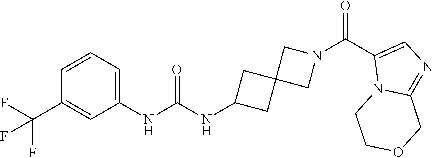 [SMILES: O=C(Nc1cccc(C(F)(F)F)c1)NC1CC2(C1)CN(C(=O)c1cnc3n1CCOC3)C2] | 1-(2-(5,6-dihydro-8H- imidazo[2,1-c][1,4]oxazine-3- carbonyl)-2- azaspiro[3.3]heptan-6-yl)-3-(3- (trifluoromethyl) phenyl)urea |
| 7 | 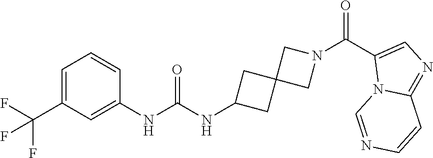 [SMILES: O=C(Nc1cccc(C(F)(F)F)c1)NC1CC2(C1)CN(C(=O)c1cnc3ccncn13)C2] | 1-(2-(imidazo[1,2- b]pyridazine-3-carbonyl)-2- azaspiro[3.3]heptan-6-yl)-3-(3- (trifluoromethyl)phenyl)urea |
| 8 | 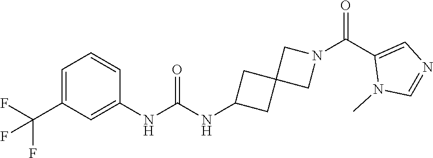 [SMILES: Cn1cncc1C(=O)N1CC2(CC(NC(=O)Nc3cccc(C(F)(F)F)c3)C2)C1] | 1-(2-(1-methyl-1H-imidazole- 5-carbonyl)-2- azaspiro[3.3]heptan-6-yl)-3-(3- (trifluoromethyl)phenyl)urea |
| 9 | 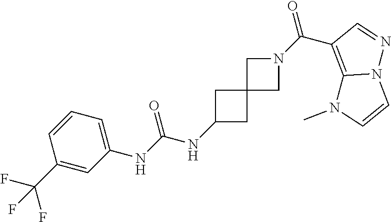 [SMILES: Cn1ccn2ncc(C(=O)N3CC4(CC(NC(=O)Nc5cccc(C(F)(F)F)c5)C4)C3)c12] | 1-(2-(1-methyl-1H- imidazo[1,2-b]pyrazole-7- carbonyl)-2- azaspiro[3.3]heptan-6-yl)-3-(3- (trifluoromethyl)phenyl)urea |
| 16 | 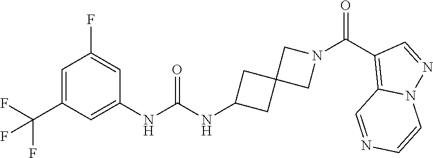 [SMILES: O=C(Nc1cc(F)cc(C(F)(F)F)c1)NC1CC2(C1)CN(C(=O)c1cnn3ccncc13)C2] | 1-(3-fluoro-5- (trifluoromethyl)phenyl)-3-(2- (pyrazolo[1,5-a]pyrazine-3- carbonyl)-2- azaspiro[3.3]heptan-6-yl)urea |
| 17 | 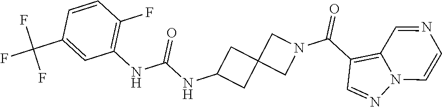 [SMILES: O=C(Nc1cc(C(F)(F)F)ccc1F)NC1CC2(C1)CN(C(=O)c1cnn3ccncc13)C2] | 1-(2-fluoro-5- (trifluoromethyl)phenyl)-3-(2- (pyrazolo[1,5-a]pyrazine-3- carbonyl)-2- azaspiro[3.3]heptan-6-yl)urea |
| 18 | 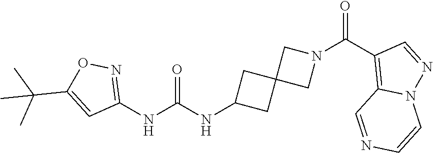 [SMILES: CC(C)(C)c1cc(NC(=O)NC2CC3(C2)CN(C(=O)c2cnn4ccncc24)C3)no1] | 1-(5-(tert-butyl)isoxazol-3-yl)- 3-(2-(pyrazolo[1,5-a]pyrazine- 3-carbonyl)-2- azaspiro[3.3]heptan-6-yl)urea |
| 19 | 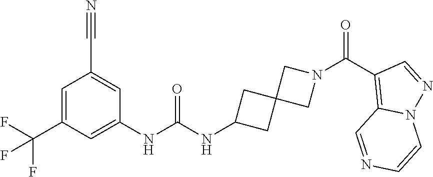 [SMILES: N#Cc1cc(NC(=O)NC2CC3(C2)CN(C(=O)c2cnn4ccncc24)C3)cc(C(F)(F)F)c1] | 1-(3-cyano-5- (trifluoromethyl)phenyl)-3-(2- (pyrazolo[1,5-a]pyrazine-3- carbonyl)-2- azaspiro[3.3]heptan-6-yl)urea |
| 20 | 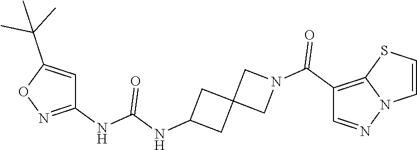 [SMILES: CC(C)(C)c1cc(NC(=O)NC2CC3(C2)CN(C(=O)c2cnn4ccsc24)C3)no1] | 1-(5-(tert-butyl)isoxazol-3-yl)- 3-(2-(pyrazolo[5,1-b]thiazole- 7-carbonyl)-2- azaspiro[3.3]heptan-6-yl)urea |
| 21 | 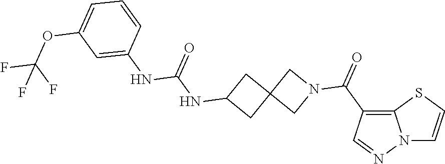 [SMILES: O=C(Nc1cccc(OC(F)(F)F)c1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(3- (trifluoromethoxy)phenyl)urea |
| 22 | 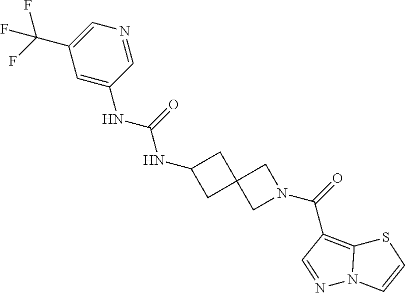 [SMILES: O=C(Nc1cncc(C(F)(F)F)c1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(5- (trifluoromethyl)pyridin-3- yl)urea |
| 23 | 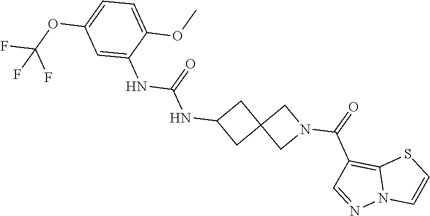 [SMILES: COc1ccc(OC(F)(F)F)cc1NC(=O)NC1CC2(C1)CN(C(=O)c1cnn3ccsc13)C2] | 1-(2-methoxy-5- (trifluoromethoxy)phenyl)-3- (2-(pyrazolo[5,1-b]thiazole-7- carbonyl)-2- azaspiro[3.3]heptan-6-yl)urea |
| 24 | 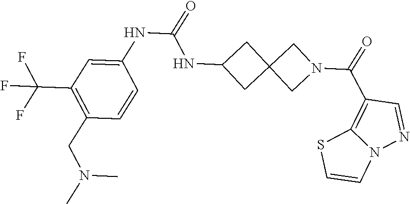 [SMILES: CN(C)Cc1ccc(NC(=O)NC2CC3(C2)CN(C(=O)c2cnn4ccsc24)C3)cc1C(F)(F)F] | 1-(4-((dimethylamino)methyl)- 3-(trifluoromethyl)phenyl)-3- (2-(pyrazolo[5,1-b]thiazole-7- carbonyl)-2- azaspiro[3.3]heptan-6-yl)urea |
| 25 | 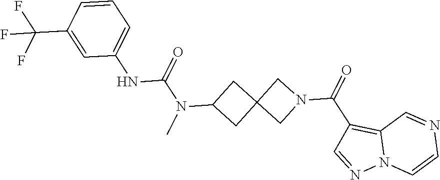 [SMILES: CN(C(=O)Nc1cccc(C(F)(F)F)c1)C1CC2(C1)CN(C(=O)c1cnn3ccncc13)C2] | 1-methyl-1-(2-(pyrazolo[1,5- a]pyrazine-3-carbonyl)-2- azaspiro[3.3]heptan-6-yl)-3-(3- (trifluoromethyl)phenyl)urea |
| 26 | 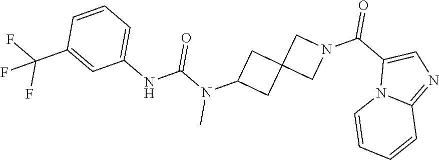 [SMILES: CN(C(=O)Nc1cccc(C(F)(F)F)c1)C1CC2(C1)CN(C(=O)c1cnc3ccccn13)C2] | 1-(2-(imidazo[1,2-a]pyridine- 3-carbonyl)-2- azaspiro[3.3]heptan-6-yl)-1- methyl-3-(3- (trifluoromethyl)phenyl)urea |
| 30 | 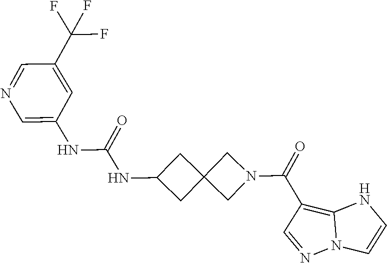 [SMILES: O=C(Nc1cncc(C(F)(F)F)c1)NC1CC2(C1)CN(C(=O)c1cnn3cc[nH]c13)C2] | 1-(2-(1H-imidazo[1,2- b]pyrazole-7-carbonyl)-2- azaspiro[3.3]heptan-6-yl)-3-(3- (trifluoromethyl)phenyl)urea |
| 31 | 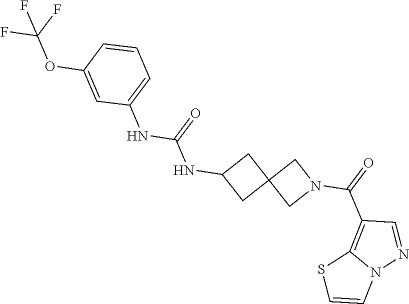 [SMILES: O=C(Nc1cccc(OC(F)(F)F)c1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(3- (2,2,2- trifluoroethyl)phenyl)urea |
| 32 | 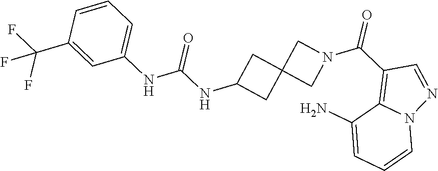 [SMILES: Nc1cccn2ncc(C(=O)N3CC4(CC(NC(=O)Nc5cccc(C(F)(F)F)c5)C4)C3)c12] | 1-(2-(4-aminopyrazolo[1,5- a]pyridine-3-carbonyl)-2- azaspiro [3.3]heptan-6-yl)-3-(3- (trifluoromethyl)phenyl)urea |
| 33 | 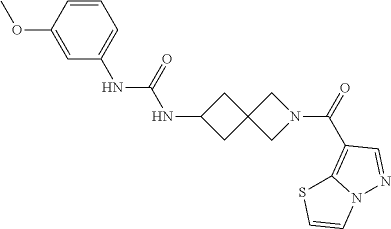 [SMILES: COc1cccc(NC(=O)NC2CC3(C2)CN(C(=O)c2cnn4ccsc24)C3)c1] | 1-(3-methoxyphenyl)-3-(2- (pyrazolo[5,1-b]thiazole-7- carbonyl)-2- azaspiro[3.3]heptan-6-yl)urea |
| 34 | 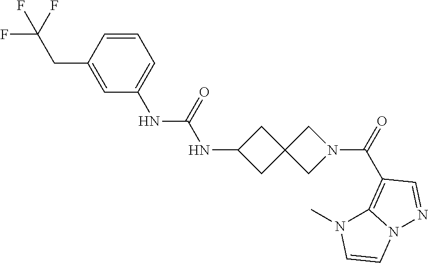 [SMILES: Cn1ccn2ncc(C(=O)N3CC4(CC(NC(=O)Nc5cccc(CC(F)(F)F)c5)C4)C3)c12] | 1-(2-(1-methyl-1H- imidazo[1,2-b]pyrazole-7- carbonyl)-2- azaspiro[3.3]heptan-6-yl)- 3-(3-(2,2,2- trifluoroethyl)phenyl)urea |
| 35 | 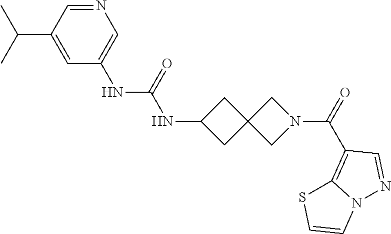 [SMILES: CC(C)c1cncc(NC(=O)NC2CC3(C2)CN(C(=O)c2cnn4ccsc24)C3)c1] | 1-(5-isopropylpyridin-3-yl)-3- (2-(pyrazolo[5,1-b]thiazole-7- carbonyl)-2- azaspiro[3.3]heptan-6-yl)urea |
| 36 | 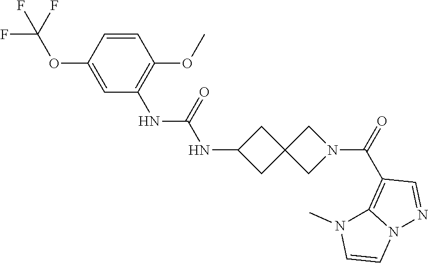 [SMILES: COc1ccc(OC(F)(F)F)cc1NC(=O)NC1CC2(C1)CN(C(=O)c1cnn3ccn(C)c13)C2] | 1-(2-methoxy-5- (trifluoromethoxy)phenyl)-3- (2-(1-methyl-1H-imidazo[1,2- b]pyrazole-7-carbonyl)-2- azaspiro[3.3]heptan-6-yl)urea |
| 37 | 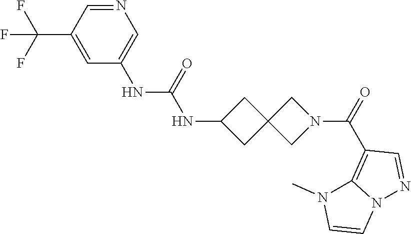 [SMILES: Cn1ccn2ncc(C(=O)N3CC4(CC(NC(=O)Nc5cncc(C(F)(F)F)c5)C4)C3)c12] | 1-(2-(1-methyl-1H- imidazo[1,2-b]pyrazole-7- carbonyl)-2-azaspiro [3.3]heptan-6-yl)-3-(5- (trifluoromethyl)pyridin-3- yl)urea |
| 38 | 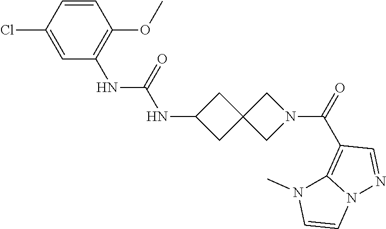 [SMILES: COc1ccc(Cl)cc1NC(=O)NC1CC2(C1)CN(C(=O)c1cnn3ccn(C)c13)C2] | 1-(5-chloro-2-methoxyphenyl)- 3-(2-(1-methyl-1H- imidazo[1,2-b]pyrazole-7- carbonyl)-2- azaspiro[3.3]heptan-6-yl)urea |
| 39 | 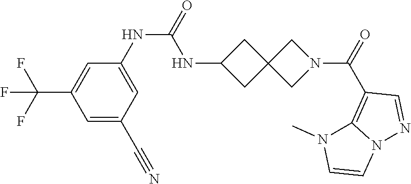 [SMILES: Cn1ccn2ncc(C(=O)N3CC4(CC(NC(=O)Nc5cc(C#N)cc(C(F)(F)F)c5)C4)C3)c12] | 1-(3-cyano-5- (trifluoromethyl)phenyl)-3-(2- (1-methyl-1H-imidazo[1,2- b]pyrazole-7-carbonyl)-2- azaspiro[3.3]heptan-6-yl)urea |
| 40 | 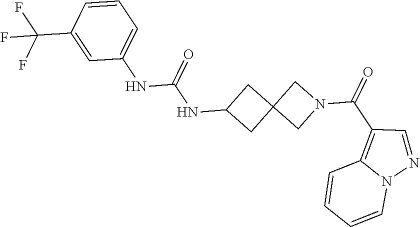 [SMILES: O=C(Nc1cccc(C(F)(F)F)c1)NC1CC2(C1)CN(C(=O)c1cnn3ccccc13)C2] | 1-(2-(pyrazolo[1,5-a]pyridine- 3-carbonyl)-2- azaspiro[3.3]heptan-6-yl)-3-(3- (trifluoromethyl)phenyl)urea |
| 41 | 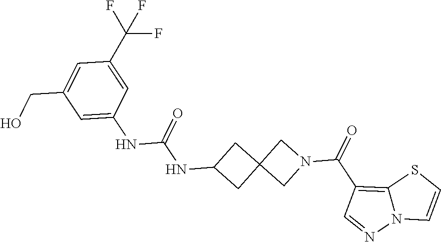 [SMILES: O=C(Nc1cc(CO)cc(C(F)(F)F)c1)NC1CC2(C1)CN(C(=O)c1cnn3ccsc13)C2] | 1-(3-(hydroxymethyl)-5- (trifluoromethyl)phenyl)-3-(2- (pyrazolo[5,1-b]thiazole-7- carbonyl)-2- azaspiro[3.3]heptan-6-yl)urea |
| 42 | 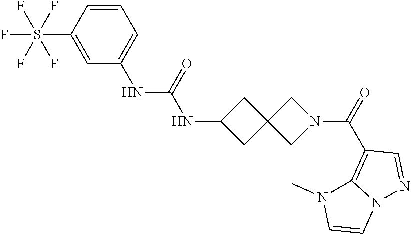 [SMILES: Cn1ccn2ncc(C(=O)N3CC4(CC(NC(=O)Nc5cccc(S(F)(F)(F)(F)F)c5)C4)C3)c12] | 1-(2-(1-methyl-1H- imidazo[1,2-b]pyrazole-7- carbonyl)-2- azaspiro[3.3]heptan-6-yl)-3-(3- (pentafluoro-16- sulfaneyl)phenyl)urea |
| 43 | 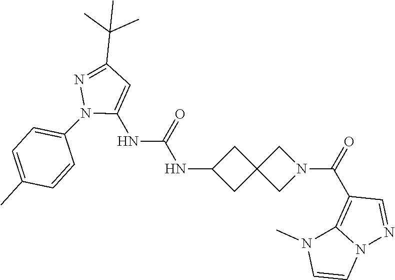 [SMILES: Cc1ccc(-n2nc(C(C)(C)C)cc2NC(=O)NC2CC3(C2)CN(C(=O)c2cnn4ccn(C)c24)C3)cc1] | 1-(3-(tert-butyl)-1-(p-tolyl)- 1H-pyrazol-5-yl)-3-(2-(1- methyl-1H-imidazo[1,2- b]pyrazole-7-carbonyl)-2- azaspiro[3.3]heptan-6-yl)urea |
| 44 | 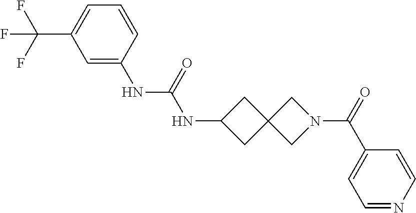 [SMILES: O=C(Nc1cccc(C(F)(F)F)c1)NC1CC2(C1)CN(C(=O)c1ccncc1)C2] | 1-(2-isonicotinoyl-2- azaspiro[3.3]heptan-6-yl)-3-(3- (trifluoromethyl)phenyl)urea |
| 45 | 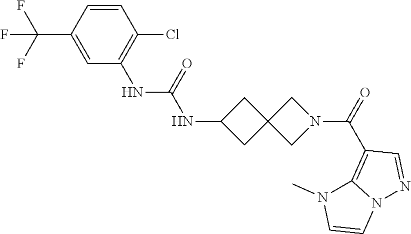 [SMILES: Cn1ccn2ncc(C(=O)N3CC4(CC(NC(=O)Nc5cc(C(F)(F)F)ccc5Cl)C4)C3)c12] | 1-(2-chloro-5- (trifluoromethyl)phenyl)-3-(2- (1-methyl-1H-imidazo[1,2- b]pyrazole-7-carbonyl)-2- azaspiro[3.3]heptan-6-yl)urea |
| 46 | 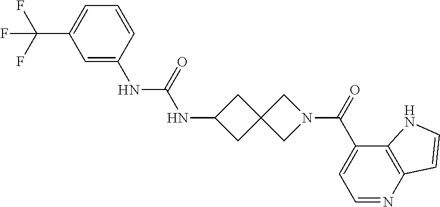 [SMILES: O=C(Nc1cccc(C(F)(F)F)c1)NC1CC2(C1)CN(C(=O)c1ccnc3cc[nH]c13)C2] | 1-(2-(1H-pyrrolo[3,2- b]pyridine-7-carbonyl)-2- azaspiro[3.3]heptan-6-yl)-3-(3- (trifluoromethyl)phenyl)urea |
| 47 | 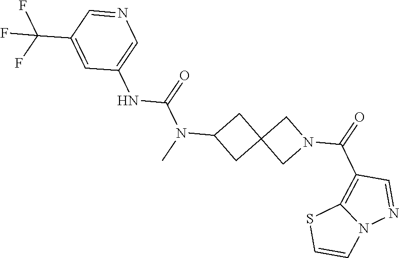 [SMILES: CN(C(=O)Nc1cncc(C(F)(F)F)c1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(5- (trifluoromethyl)pyridin-3- yl)urea |
| 48 | 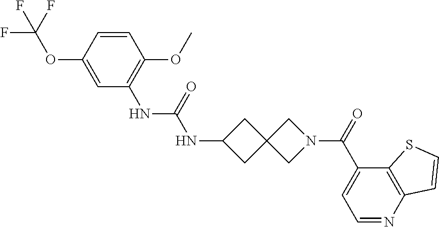 [SMILES: COc1ccc(OC(F)(F)F)cc1NC(=O)NC1CC2(C1)CN(C(=O)c1ccnc3ccsc13)C2] | 1-(2-methoxy-5- (trifluoromethoxy)phenyl)-3- (2-(thieno[3,2-b]pyridine-7- carbonyl)-2- azaspiro[3.3]heptan-6-yl)urea |
| 49 | 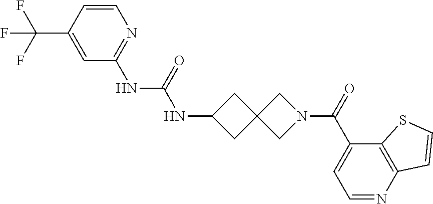 [SMILES: O=C(Nc1cc(C(F)(F)F)ccn1)NC1CC2(C1)CN(C(=O)c1ccnc3ccsc13)C2] | 1-(2-(thieno[3,2-b]pyridine-7- carbonyl)-2- azaspiro[3.3]heptan-6-yl)-3-(5- (trifluoromethyl)pyridin-3- yl)urea |
| 50 | 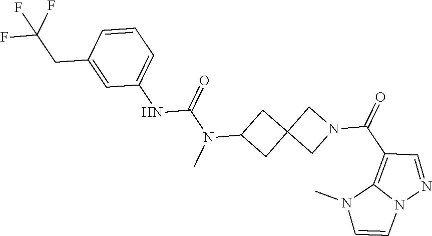 [SMILES: CN(C(=O)Nc1cccc(CC(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)- 3-(3-(2,2,2- trifluoroethyl)phenyl)urea |
| 51 | 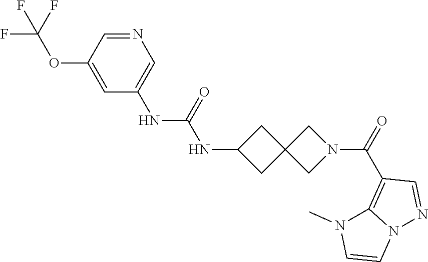 [SMILES: Cn1ccn2ncc(C(=O)N3CC4(CC(NC(=O)Nc5cncc(OC(F)(F)F)c5)C4)C3)c12] | 1-(2-(1-methyl-1H- imidazo[1,2-b]pyrazole-7- carbonyl)-2- azaspiro[3.3]heptan-6-yl)-3-(5- (trifluoromethoxy)pyridin-3- yl)urea |
| 52 | 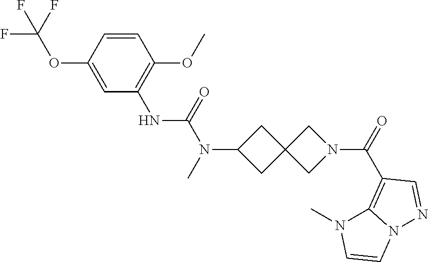 [SMILES: COc1ccc(OC(F)(F)F)cc1NC(=O)N(C)C1CC2(C1)CN(C(=O)c1cnn3ccn(C)c13)C2] | 3-(2-methoxy-5- (trifluoromethoxy)phenyl)-1- methyl-1-(2-(1-methyl-1H- imidazo[1,2-b]pyrazole-7- carbonyl)-2- azaspiro[3.3]heptan-6-yl)urea |
| 53 | 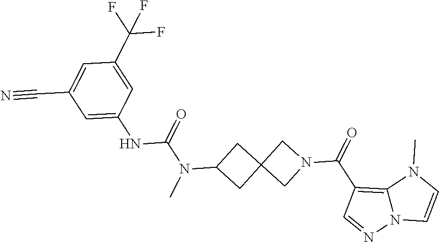 [SMILES: CN(C(=O)Nc1cc(C#N)cc(C(F)(F)F)c1)C1CC2(C1)CN(C(=O)c1cnn3ccn(C)c13)C2] | 3-(3-cyano-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 54 | 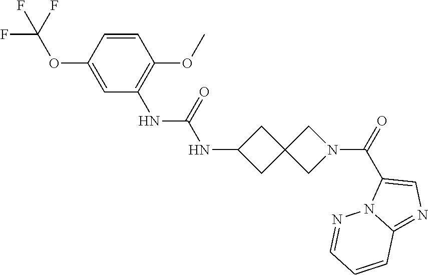 [SMILES: COc1ccc(OC(F)(F)F)cc1NC(=O)NC1CC2(C1)CN(C(=O)c1cnc3cccnn13)C2] | 1-(2-(imidazo[1,2- b]pyridazine-3-carbonyl)-2- azaspiro[3.3]heptan-6-yl)-3-(2- methoxy-5- (trifluoromethoxy)phenyl)urea |
| 55 | 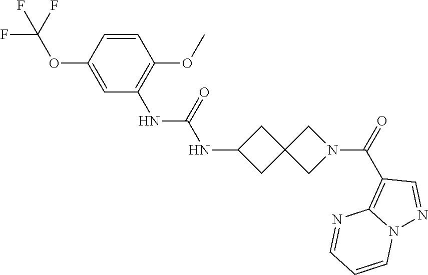 [SMILES: COc1ccc(OC(F)(F)F)cc1NC(=O)NC1CC2(C1)CN(C(=O)c1cnn3cccnc13)C2] | 1-(2-methoxy-5- (trifluoromethoxy)phenyl)-3- (2-(pyrazolo[1,5-a]pyrimidine- 3-carbonyl)-2- azaspiro[3.3]heptan-6-yl)urea |
| 56 | 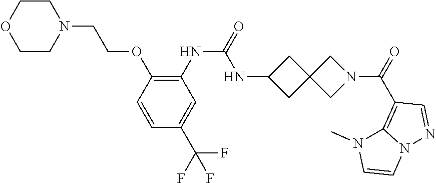 [SMILES: Cn1ccn2ncc(C(=O)N3CC4(CC(NC(=O)Nc5cc(C(F)(F)F)ccc5OCCN5CCOCC5)C4)C3)c12] | 1-(2-(1-methyl-1H- imidazo[1,2-b]pyrazole-7- carbonyl)-2- azaspiro[3.3]heptan-6-yl)-3-(2- (2-morpholinoethoxy)-5- (trifluoromethyl)phenyl)urea |
| 57 | 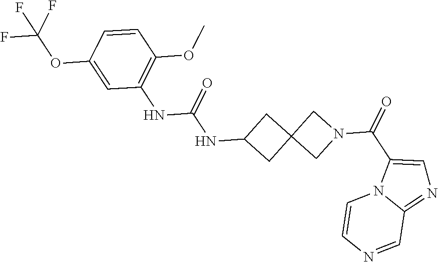 [SMILES: COc1ccc(OC(F)(F)F)cc1NC(=O)NC1CC2(C1)CN(C(=O)c1cnc3cnccn13)C2] | 1-(2-(imidazo[1,2-a]pyrazine- 3-carbonyl)-2- azaspiro[3.3]heptan-6-yl)-3-(2- methoxy-5- (trifluoromethoxy)phenyl)urea |
| 58 | 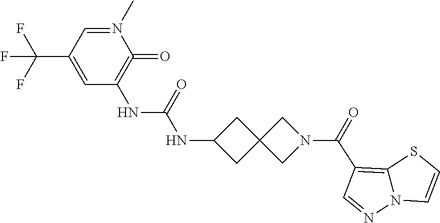 | 1-(1-methyl-2-oxo-5- (trifluoromethyl)-1,2- dihydropyridin-3-yl)-3-(2- (pyrazolo[5,1-b]thiazole-7- carbonyl)-2- azaspiro[3.3]heptan-6-yl)urea |
| 59 | 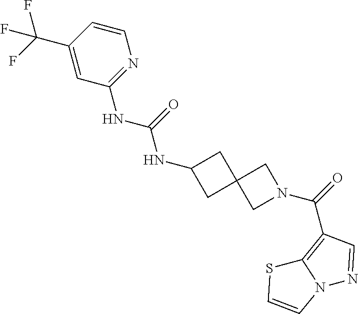 [SMILES: O=C(Nc1cc(C(F)(F)F)ccn1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(4- (trifluoromethyl)pyridin-2- yl)urea |
| 60 | 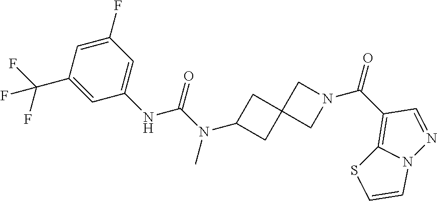 [SMILES: CN(C(=O)Nc1cc(F)cc(C(F)(F)F)c1)C1CC2(C1)CN(C(=O)c1cnn3ccsc13)C2] | 3-(3-fluoro-5-(trifluoro methyl)phenyl)-1-methyl-1-(2- (pyrazolo[5,1-b]thiazole-7- carbonyl)-2- azaspiro[3.3]heptan-6-yl)urea |
| 61 | 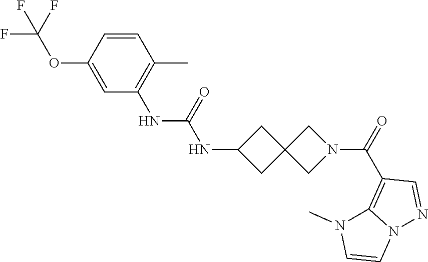 [SMILES: Cc1ccc(OC(F)(F)F)cc1NC(=O)NC1CC2(C1)CN(C(=O)c1cnn3ccn(C)c13)C2] | 1-(2-(1-methyl-1H- imidazo[1,2-b]pyrazole-7- carbonyl)-2- azaspiro[3.3]heptan-6-yl)-3-(2- methyl-5- (trifluoromethoxy)phenyl)urea |
| 62 | 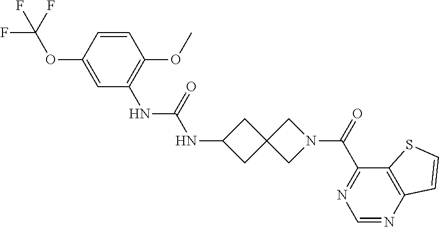 [SMILES: COc1ccc(OC(F)(F)F)cc1NC(=O)NC1CC2(C1)CN(C(=O)c1ncnc3ccsc13)C2] | 1-(2-methoxy-5- (trifluoromethoxy)phenyl)-3- (2-(thieno[3,2-d]pyrimidine-4- carbonyl)-2- azaspiro[3.3]heptan-6-yl)urea |
| 63 | 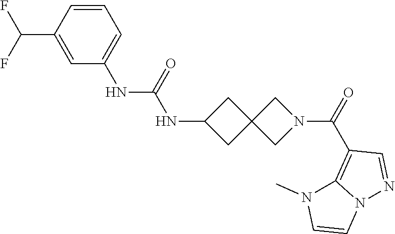 [SMILES: Cn1ccn2ncc(C(=O)N3CC4(CC(NC(=O)Nc5cccc(C(F)F)c5)C4)C3)c12] | 1-(3-(difluoromethyl)phenyl)- 3-(2-(1-methyl-1H- imidazo[1,2-b]pyrazole-7- carbonyl)-2- azaspiro[3.3]heptan-6-yl)urea |
| 64 | 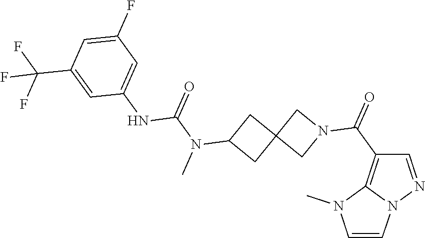 [SMILES: CN(C(=O)Nc1cc(F)cc(C(F)(F)F)c1)C1CC2(C1)CN(C(=O)c1cnn3ccn(C)c13)C2] | 3-(3-fluoro-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 65 | 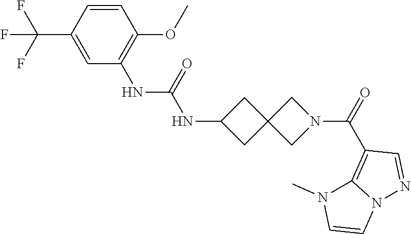 [SMILES: COc1ccc(C(F)(F)F)cc1NC(=O)NC1CC2(C1)CN(C(=O)c1cnn3ccn(C)c13)C2] | 1-(2-methoxy-5- (trifluoromethyl)phenyl)-3-(2- (1-methyl-1H-imidazo[1,2- b]pyrazole-7-carbonyl)-2- azaspiro[3.3]heptan-6-yl)urea |
| 66 | 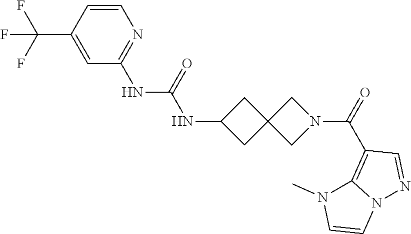 [SMILES: Cn1ccn2ncc(C(=O)N3CC4(CC(NC(=O)Nc5cc(C(F)(F)F)ccn5)C4)C3)c12] | 1-(4-(difluoromethyl)pyridin- 2-yl)-3-(2-(1-methyl-1H- imidazo[1,2-b]pyrazole-7- carbonyl)-2- azaspiro[3.3]heptan-6-yl)urea |
| 67 | 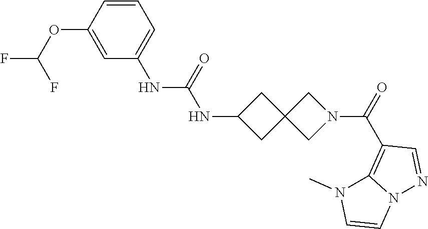 [SMILES: Cn1ccn2ncc(C(=O)N3CC4(CC(NC(=O)Nc5cccc(OC(F)F)c5)C4)C3)c12] | 1-(3- (difluoromethoxy)phenyl)-3- (2-(1-methyl-1H-imidazo[1,2- b]pyrazole-7-carbonyl)-2- azaspiro[3.3]heptan-6-yl)urea |
| 68 | 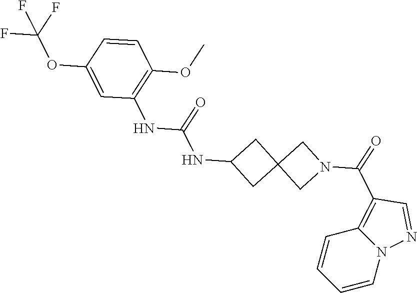 [SMILES: COc1ccc(OC(F)(F)F)cc1NC(=O)NC1CC2(C1)CN(C(=O)c1cnn3ccccc13)C2] | 1-(2-methoxy-5- (trifluoromethoxy)phenyl)-3- (2-(pyrazolo[1,5-a]pyridine-3- carbonyl)-2- azaspiro[3.3]heptan-6-yl)urea |
| 69 | 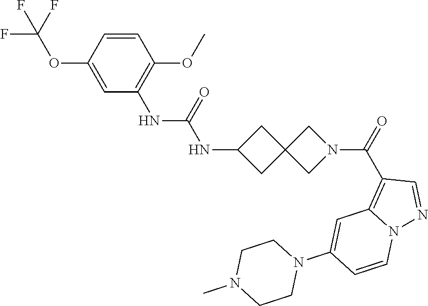 [SMILES: COc1ccc(OC(F)(F)F)cc1NC(=O)NC1CC2(C1)CN(C(=O)c1cnn3ccc(N4CCN(C)CC4)cc13)C2] | 1-(2-methoxy-5- (trifluoromethoxy)phenyl)-3- (2-(5-(4-methylpiperazin-1- yl)pyrazolo[1,5-a]pyridine-3- carbonyl)-2- azaspiro[3.3]heptan-6-yl)urea |
| 70 | 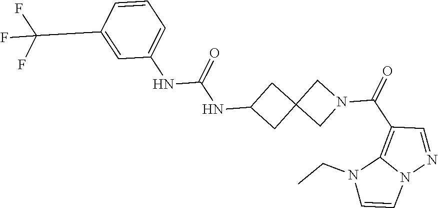 [SMILES: CCn1ccn2ncc(C(=O)N3CC4(CC(NC(=O)Nc5cccc(CC(F)(F)F)c5)C4)C3)c12] | 1-(2-(1-ethyl-1H-imidazo[1,2- b]pyrazole-7-carbonyl)-2- azaspiro[3.3]heptan-6-yl)-3-(3- (trifluoromethyl)phenyl)urea |
| 71 | 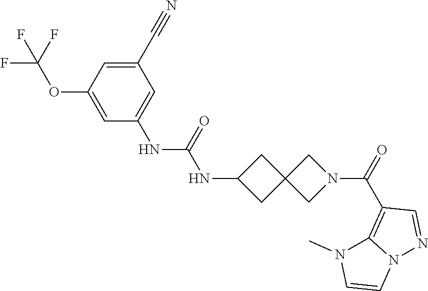 [SMILES: Cn1ccn2ncc(C(=O)N3CC4(CC(NC(=O)Nc5cc(C#N)cc(OC(F)(F)F)c5)C4)C3)c12] | 1-(3-cyano-5- (trifluoromethoxy)phenyl)-3- (2-(1-methyl-1H-imidazo[1,2- b]pyrazole-7-carbonyl)-2- azaspiro[3.3]heptan-6-yl)urea |
| 72 | 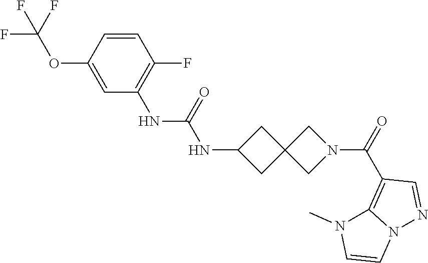 [SMILES: Cn1ccn2ncc(C(=O)N3CC4(CC(NC(=O)Nc5cc(OC(F)(F)F)ccc5F)C4)C3)c12] | 1-(2-fluoro-5- (trifluoromethoxy)phenyl)-3- (2-(1-methyl-1H-imidazo[1,2- b]pyrazole-7-carbonyl)-2- azaspiro[3.3]heptan-6-yl)urea |
| 73 | 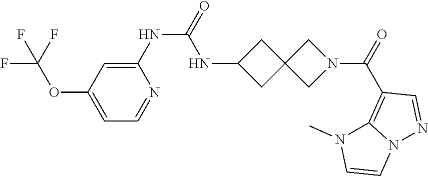 [SMILES: Cn1ccn2ncc(C(=O)N3CC4(CC(NC(=O)Nc5cc(OC(F)(F)F)ccn5)C4)C3)c12] | 1-(2-(1-methyl-1H- imidazo[1,2-b]pyrazole-7- carbonyl)-2- azaspiro[3.3]heptan-6-yl)-3-(4- (trifluoromethoxy)pyridin-2- yl)urea |
| 74 | 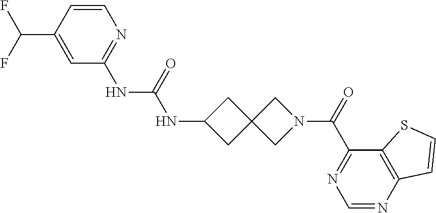 [SMILES: O=C(Nc1cc(C(F)F)ccn1)NC1CC2(C1)CN(C(=O)c1ncnc3ccsc13)C2] | 1-(4-(difluoromethyl)pyridin- 2-yl)-3-(2-(thieno[3,2- d]pyrimidine-4-carbonyl)-2- azaspiro[3.3]heptan-6-yl)urea |
| 75 | 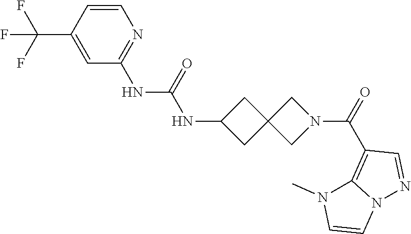 [SMILES: Cn1ccn2ncc(C(=O)N3CC4(CC(NC(=O)Nc5cc(C(F)(F)F)ccn5)C4)C3)c12] | 1-(2-(1-methyl-1H- imidazo[1,2-b]pyrazole-7- carbonyl)-2- azaspiro[3.3]heptan-6-yl)-3-(4- (trifluoromethyl)pyridin-2- yl)urea |
| 76 | 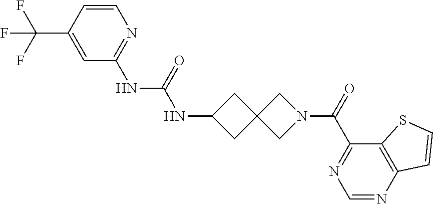 [SMILES: O=C(Nc1cc(C(F)(F)F)ccn1)NC1CC2(C1)CN(C(=O)c1ncnc3ccsc13)C2] | 1-(2-(thieno[3,2-d]pyrimidine- 4-carbonyl)-2- azaspiro[3.3]heptan-6-yl)-3-(5- (trifluoromethyl)pyridin-3- yl)urea |
| 77 | 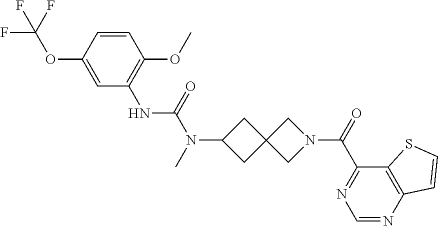 [SMILES: COc1ccc(OC(F)(F)F)cc1NC(=O)N(C)C1CC2(C1)CN(C(=O)c1ncnc3ccsc13)C2] | 3-(2-methoxy-5- (trifluoromethoxy)phenyl)-1- methyl-1-(2-(thieno[3,2- d]pyrimidine-4-carbonyl)-2- azaspiro[3.3]heptan-6-yl)urea |
| 78 | 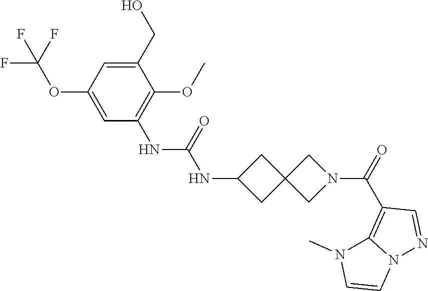 [SMILES: COc1c(CO)cc(OC(F)(F)F)cc1NC(=O)NC1CC2(C1)CN(C(=O)c1cnn3ccn(C)c13)C2] | 1-(3-(hydroxymethyl)-2- methoxy-5- (trifluoromethoxy)phenyl)-3- (2-(1-methyl-1H-imidazo[1,2- b]pyrazole-7-carbonyl)-2- azaspiro[3.3]heptan-6-yl)urea |
| 79 | 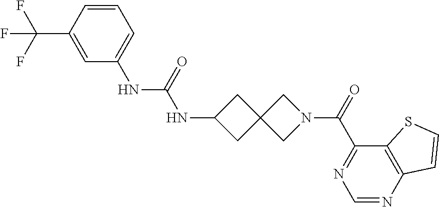 [SMILES: O=C(Nc1cccc(C(F)(F)F)c1)NC1CC2(C1)CN(C(=O)c1ncnc3ccsc13)C2] | 1-(2-(thieno[3,2-d]pyrimidine- 4-carbonyl)-2- azaspiro[3.3]heptan-6-yl)-3-(3- (trifluoromethyl)phenyl)urea |
| 80 | 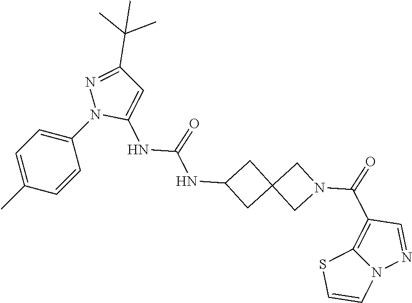 [SMILES: Cc1ccc(-n2nc(C(C)(C)C)cc2NC(=O)NC2CC3(C2)CN(C(=O)c2cnn4ccsc24)C3)cc1] | 1-(3-(tert-butyl)-1-(p-tolyl)- 1H-pyrazol-5-yl)-3-(2- (pyrazolo[5,1-b]thiazole-7- carbonyl)-2- azaspiro[3.3]heptan-6-yl)urea |
| 81 | 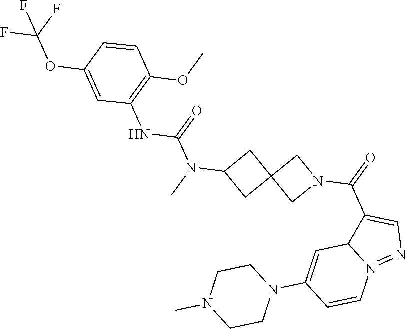 | 3-(2-methoxy-5- (trifluoromethoxy)phenyl)-1- methyl-1-(2-(5-(4- methylpiperazin-1- yl)pyrazolo[1,5-a]pyridine-3- carbonyl)-2- azaspiro[3.3]heptan-6-yl)urea |
| 82 | 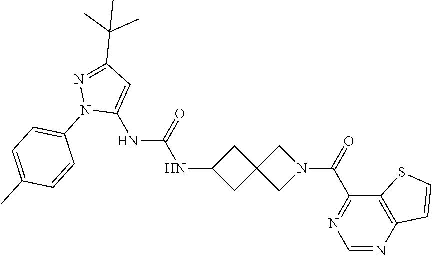 [SMILES: Cc1ccc(-n2nc(C(C)(C)C)cc2NC(=O)NC2CC3(C2)CN(C(=O)c2ncnc4ccsc24)C3)cc1] | 1-(3-(tert-butyl)-1-(p-tolyl)- 1H-pyrazol-5-yl)-3-(2- (thieno[3,2-d]pyrimidine-4- carbonyl)-2- azaspiro[3.3]heptan-6-yl)urea |
| 83 | 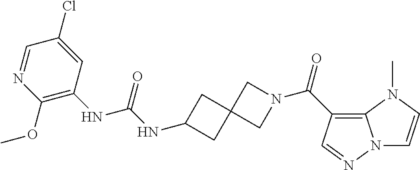 [SMILES: COc1ncc(Cl)cc1NC(=O)NC1CC2(C1)CN(C(=O)c1cnn3ccn(C)c13)C2] | 1-(5-chloro-2-methoxypyridin- 3-yl)-3-(2-(1-methyl-1H- imidazo[1,2-b]pyrazole-7- carbonyl)-2- azaspiro[3.3]heptan-6-yl)urea |
| 84 | 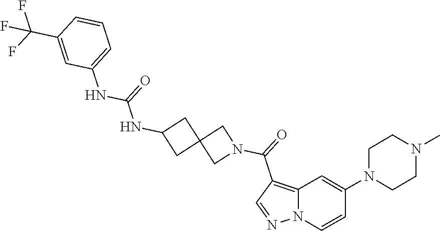 [SMILES: CN1CCN(c2ccn3ncc(C(=O)N4CC5(CC(NC(=O)Nc6cccc(C(F)(F)F)c6)C5)C4)c3c2)CC1] | 1-(2-(5-(4-methylpiperazin-1- yl)pyrazolo[1,5-a]pyridine-3- carbonyl)-2- azaspiro[3.3]heptan-6-yl)-3-(3- (trifluoromethyl)phenyl)urea |
| 85 | 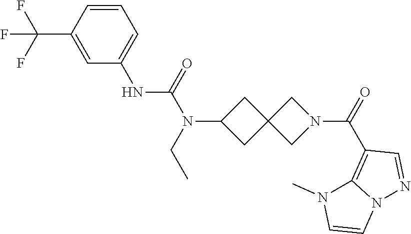 [SMILES: CCN(C(=O)Nc1cccc(C(F)(F)F)c1)C1CC2(C1)CN(C(=O)c1cnn3ccn(C)c13)C2] | 1-ethyl-1-(2-(1-methyl-1H- imidazo[1,2-b]pyrazole-7- carbonyl)-2- azaspiro[3.3]heptan-6-yl)-3-(3- (trifluoromethyl)phenyl)urea |
| 86 | 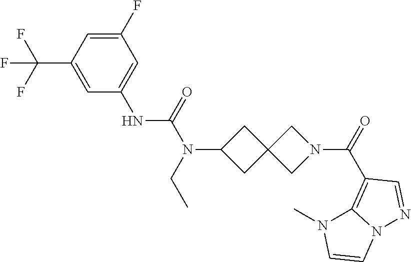 [SMILES: CCN(C(=O)Nc1cc(F)cc(C(F)(F)F)c1)C1CC2(C1)CN(C(=O)c1cnn3ccn(C)c13)C2] | 1-ethyl-3-(3-fluoro-5- (trifluoromethyl)phenyl)-1-(2- (1-methyl-1H-imidazo[1,2- b]pyrazole-7-carbonyl)-2- azaspiro[3.3]heptan-6-yl)urea |
| 87 | 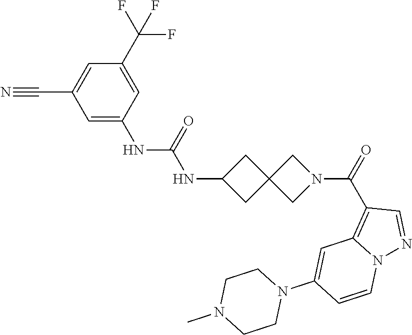 [SMILES: CN1CCN(c2ccn3ncc(C(=O)N4CC5(CC(NC(=O)Nc6cc(C#N)cc(C(F)(F)F)c6)C5)C4)c3c2)CC1] | 1-(3-cyano-5- (trifluoromethyl)phenyl)-3-(2- (5-(4-methylpiperazin-1- yl)pyrazolo[1,5-a]pyridine-3- carbonyl)-2- azaspiro[3.3]heptan-6-yl)urea |
| 88 | 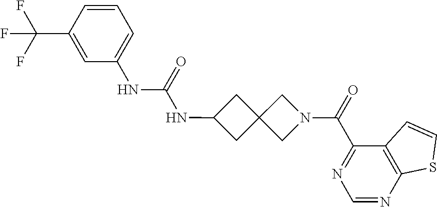 [SMILES: O=C(Nc1cccc(C(F)(F)F)c1)NC1CC2(C1)CN(C(=O)c1ncnc3sccc13)C2] | 1-(2-(thieno[2,3-d]pyrimidine- 4-carbonyl)-2- azaspiro[3.3]heptan-6-yl)-3-(3- (trifluoromethyl)phenyl)urea |
| 89 | 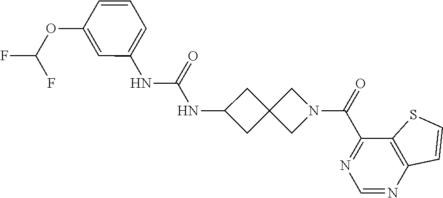 [SMILES: O=C(Nc1cccc(OC(F)F)c1)NC1CC2(C1)CN(C(=O)c1ncnc3ccsc13)C2] | 1-(3- (difluoromethoxy)phenyl)-3- (2-(thieno[3,2-d]pyrimidine-4- carbonyl)-2- azaspiro[3.3]heptan-6-yl)urea |
| 90 | 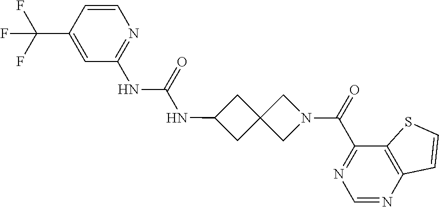 [SMILES: O=C(Nc1cc(C(F)(F)F)ccn1)NC1CC2(C1)CN(C(=O)c1ncnc3ccsc13)C2] | 1-(2-(thieno[3,2-d]pyrimidine- 4-carbonyl)-2- azaspiro[3.3]heptan-6-yl)-3-(4- (trifluoromethyl)pyridin-2- yl)urea |
| 91 | 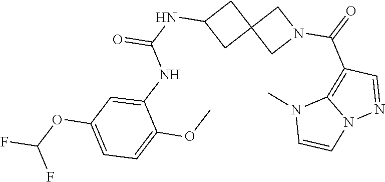 [SMILES: COc1ccc(OC(F)F)cc1NC(=O)NC1CC2(C1)CN(C(=O)c1cnn3ccn(C)c13)C2] | 1-(5-(difluoromethoxy)-2- methoxyphenyl)-3-(2-(1- methyl-1H-imidazo[1,2- b]pyrazole-7-carbonyl)-2- azaspiro[3.3]heptan-6-yl)urea |
| 92 | 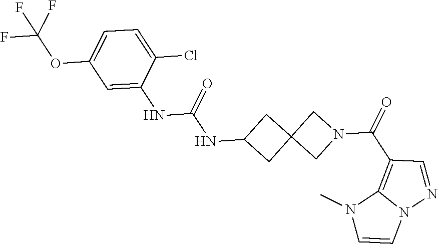 [SMILES: Cn1ccn2ncc(C(=O)N3CC4(CC(NC(=O)Nc5cc(OC(F)(F)F)ccc5Cl)C4)C3)c12] | 1-(2-chloro-5- (trifluoromethoxy)phenyl)-3- (2-(1-methyl-1H-imidazo[1,2- b]pyrazole-7-carbonyl)-2- azaspiro[3.3]heptan-6-yl)urea |
| 93 | 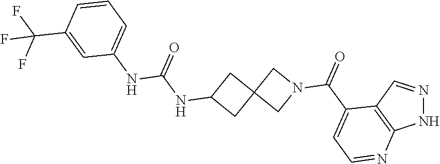 [SMILES: O=C(Nc1cccc(C(F)(F)F)c1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(3- (trifluoromethyl)phenyl)urea |
| 94 | 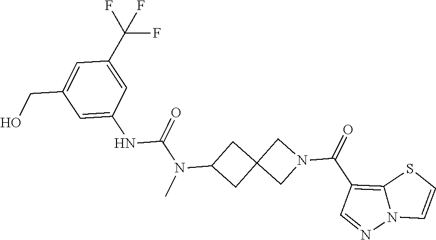 [SMILES: CN(C(=O)Nc1cc(CO)cc(C(F)(F)F)c1)C1CC2(C1)CN(C(=O)c1cnn3ccsc13)C2] | 3-(3-(hydroxymethyl)-5- (trifluoromethyl)phenyl)-1- methyl-1-(2-(pyrazolo[5,1- b]thiazole-7-carbonyl)-2- azaspiro[3.3]heptan-6-yl)urea |
| 95 | 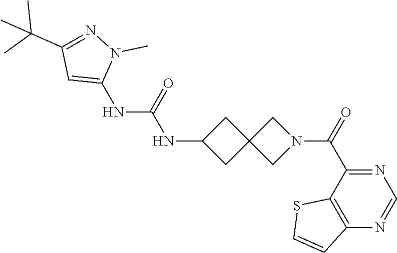 [SMILES: Cn1nc(C(C)(C)C)cc1NC(=O)NC1CC2(C1)CN(C(=O)c1ncnc3ccsc13)C2] | 1-(3-(tert-butyl)-1-methyl-1H- pyrazol-5-yl)-3-(2-(thieno[3,2- d]pyrimidine-4-carbonyl)-2- azaspiro[3.3]heptan-6-yl)urea |
| 96 | 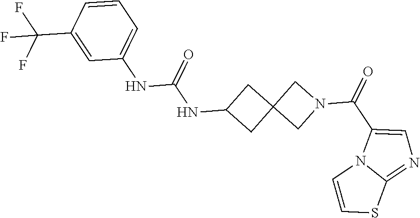 [SMILES: O=C(Nc1cccc(C(F)(F)F)c1)NC1CC2(C1)CN(C(=O)c1cnc3sccn13)C2] | 1-(2-(imidazo[2,1-b]thiazole-5- carbonyl)-2- azaspiro[3.3]heptan-6-yl)-3-(3- (trifluoromethyl)phenyl)urea |
| 97 | 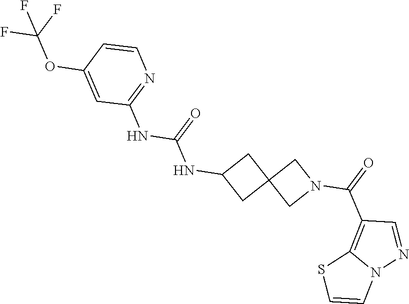 [SMILES: O=C(Nc1cc(OC(F)(F)F)ccn1)NC1CC2(C1)CN(C(=O)c1cnn3ccsc13)C2] | 1-(2-(pyrazolo[5,1-b]thiazole- 7-carbonyl)-2- azaspiro[3.3]heptan-6-yl)-3-(4- (trifluoromethoxy)pyridin-2- yl)urea |
| 98 | 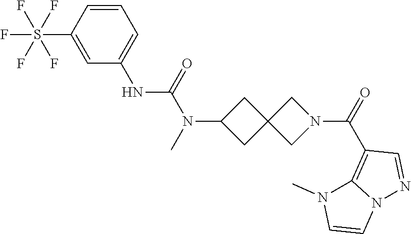 [SMILES: CN(C(=O)Nc1cccc(S(F)(F)(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(3- (pentafluoro-16- sulfaneyl)phenyl)urea |
| 99 | 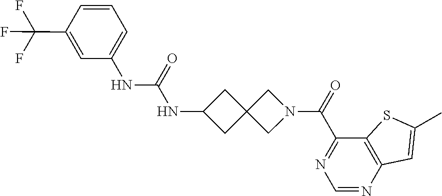 [SMILES: Cc1cc2ncnc(C(=O)N3CC4(CC(NC(=O)Nc5cccc(C(F)(F)F)c5)C4)C3)c2s1] | 1-(2-(6-methylthieno[3,2- d]pyrimidine-4-carbonyl)-2- azaspiro[3.3]heptan-6-yl)-3-(3- (trifluoromethyl)phenyl)urea |
| 100 | 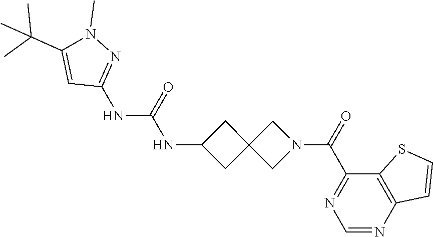 [SMILES: Cn1nc(NC(=O)NC2CC3(C2)CN(C(=O)c2ncnc4ccsc24)C3)cc1C(C)(C)C] | 1-(5-(tert-butyl)-1-methyl-1H- pyrazol-3-yl)-3-(2-(thieno[3,2- d]pyrimidine-4-carbonyl)-2- azaspiro[3.3]heptan-6-yl)urea |
| 101 | 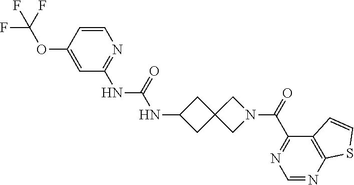 [SMILES: O=C(Nc1cc(OC(F)(F)F)ccn1)NC1CC2(C1)CN(C(=O)c1ncnc3sccc13)C2] | 1-(2-(thieno[3,2-d]pyrimidine- 4-carbonyl)-2- azaspiro[3.3]heptan-6-yl)-3-(4- (trifluoromethoxy)pyridin-2- yl)urea |
| 102 | 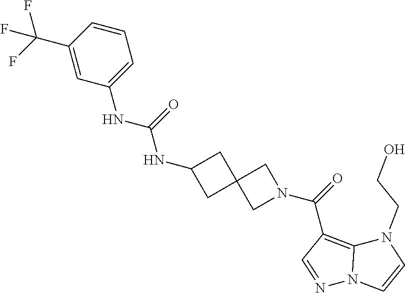 [SMILES: O=C(Nc1cccc(C(F)(F)F)c1)NC1CC2(C1)CN(C(=O)c1cnn3ccn(CCO)c13)C2] | 1-(2-(1-(2-hydroxyethyl)-1H- imidazo[1,2-b]pyrazole-7- carbonyl)-2- azaspiro[3.3]heptan-6-yl)-3-(3- (trifluoromethyl)phenyl)urea |
| 103 | 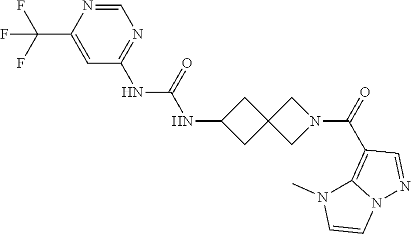 [SMILES: Cn1ccn2ncc(C(=O)N3CC4(CC(NC(=O)Nc5cc(C(F)(F)F)ncn5)C4)C3)c12] | 1-(2-(1-methyl-1H- imidazo[1,2-b]pyrazole-7- carbonyl)-2- azaspiro[3.3]heptan-6-yl)-3-(6- (trifluoromethyl)pyrimidin-4- yl)urea |
| 104 | 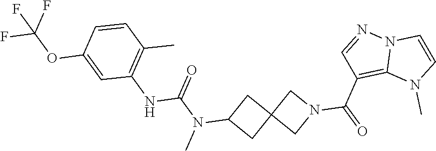 [SMILES: Cc1ccc(OC(F)(F)F)cc1NC(=O)N(C)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(2- methyl-5- (trifluoromethoxy)phenyl)urea |
| 105 | 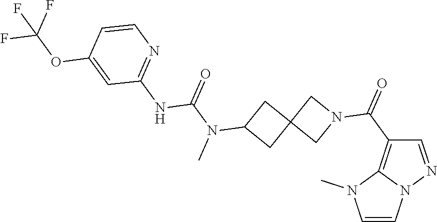 [SMILES: CN(C(=O)Nc1cc(OC(F)(F)F)ccn1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(4- (trifluoromethoxy)pyridin-2- yl)urea |
| 106 | 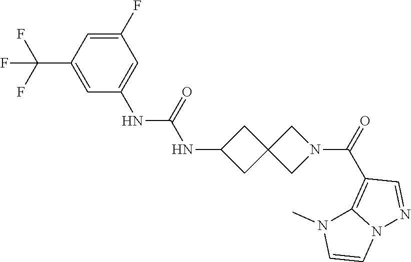 [SMILES: Cn1ccn2ncc(C(=O)N3CC4(CC(NC(=O)Nc5cc(F)cc(C(F)(F)F)c5)C4)C3)c12] | 1-(3-fluoro-5- (trifluoromethyl)phenyl)-3-(2- (1-methyl-1H-imidazo[1,2- b]pyrazole-7-carbonyl)-2- azaspiro[3.3]heptan-6-yl)urea |
| 107 | 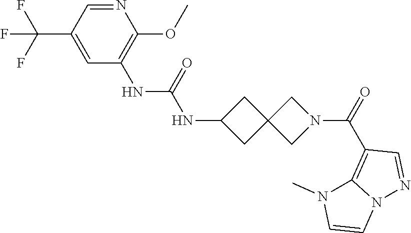 [SMILES: COc1ncc(C(F)(F)F)cc1NC(=O)NC1CC2(C1)CN(C(=O)c1cnn3ccn(C)c13)C2] | 1-(2-methoxy-5- (trifluoromethyl)pyridin-3-yl)- 3-(2-(1-methyl-1H- imidazo[1,2-b]pyrazole-7- carbonyl)-2- azaspiro[3.3]heptan-6-yl)urea |
| 108 | 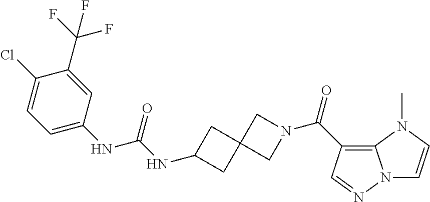 [SMILES: Cn1ccn2ncc(C(=O)N3CC4(CC(NC(=O)Nc5ccc(Cl)c(C(F)(F)F)c5)C4)C3)c12] | 1-(4-chloro-3- (trifluoromethyl)phenyl)-3-(2- (1-methyl-1H-imidazo[1,2- b]pyrazole-7-carbonyl)-2- azaspiro[3.3]heptan-6-yl)urea |
| 109 | 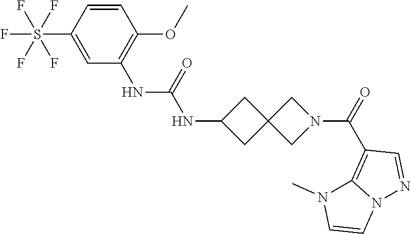 [SMILES: COc1ccc(S(F)(F)(F)(F)F)cc1NC(=O)NC1CC2(C1)CN(C(=O)c1cnn3ccn(C)c13)C2] | 1-(2-methoxy-5-(pentafluoro- 16-sulfaneyl)phenyl)-3-(2-(1- methyl-1H-imidazo[1,2- b]pyrazole-7-carbonyl)-2- azaspiro[3.3]heptan-6-yl)urea |
| 110 | 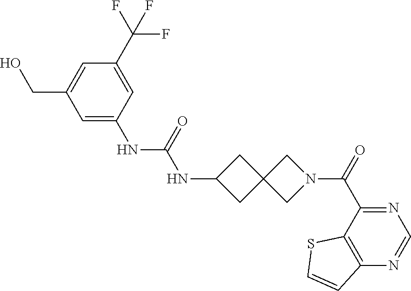 [SMILES: O=C(Nc1cc(CO)cc(C(F)(F)F)c1)NC1CC2(C1)CN(C(=O)c1ncnc3ccsc13)C2] | 1-(3-(hydroxymethyl)-5- (trifluoromethyl)phenyl)-3-(2- (thieno[3,2-d]pyrimidine-4- carbonyl)-2- azaspiro[3.3]heptan-6-yl)urea |
| 111 | 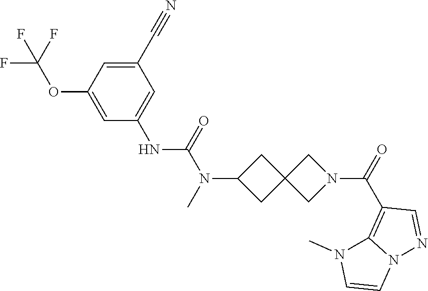 [SMILES: CN(C(=O)Nc1cc(C#N)cc(OC(F)(F)F)c1)C1CC2(C1)CN(C(=O)c1cnn3ccn(C)c13)C2] | 3-(3-cyano-5- (trifluoromethoxy)phenyl)-1- methyl-1-(2-(1-methyl-1H- imidazo[1,2-b]pyrazole-7- carbonyl)-2- azaspiro[3.3]heptan-6-yl)urea |
| 112 | 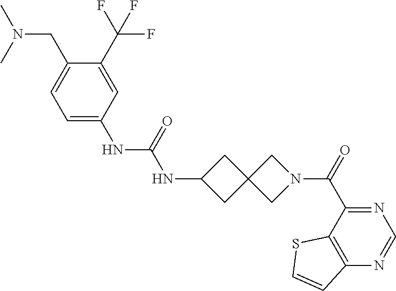 [SMILES: CN(C)Cc1ccc(NC(=O)NC2CC3(C2)CN(C(=O)c2ncnc4ccsc24)C3)cc1C(F)(F)F] | 1-(4-((dimethylamino)methyl)- 3-(trifluoromethyl)phenyl)-3- (2-(thieno[3,2-d]pyrimidine-4- carbonyl)-2- azaspiro[3.3]heptan-6-yl)urea |
| 113 | 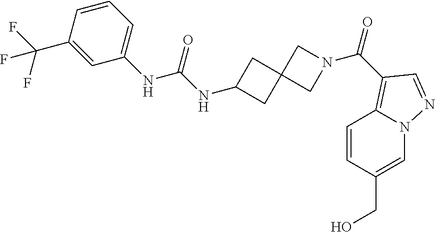 [SMILES: O=C(Nc1cccc(C(F)(F)F)c1)NC1CC2(C1)CN(C(=O)c1cnn3cc(CO)ccc13)C2] | 1-(2-(6- (hydroxymethyl)pyrazolo[1,5- a]pyridine-3-carbonyl)-2- azaspiro[3.3]heptan-6-yl)-3-(3- (trifluoromethyl)phenyl)urea |
| 114 | 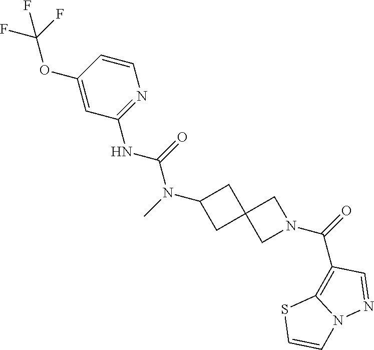 [SMILES: CN(C(=O)Nc1cc(OC(F)(F)F)ccn1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(4- (trifluoromethoxy)pyridin-2- yl)urea |
| 115 | 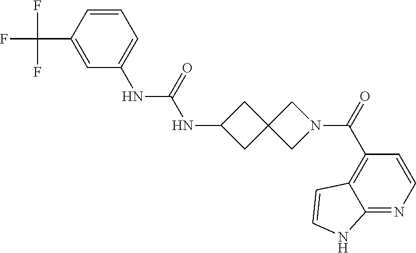 [SMILES: O=C(Nc1cccc(C(F)(F)F)c1)NC1CC2(C1)CN(C(=O)c1ccnc3[nH]ccc13)C2] | 1-(2-(1H-pyrrolo[2,3- b]pyridine-4-carbonyl)-2- azaspiro[3.3]heptan-6-yl)-3-(3- (trifluoromethyl)phenyl)urea |
| 116 | 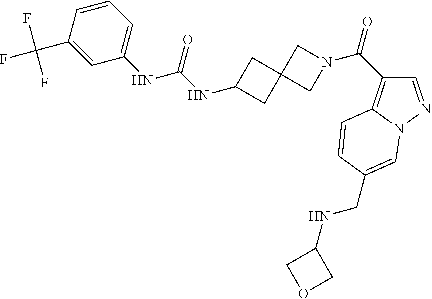 [SMILES: O=C(Nc1cccc(C(F)(F)F)c1)NC1CC2(C1)CN(C(=O)c1cnn3cc(CNC4COC4)ccc13)C2] | 1-(2-(6-((oxetan-3- ylamino)methyl)pyrazolo[1,5- a]pyridine-3-carbonyl)-2- azaspiro[3.3]heptan-6-yl)-3-(3- (trifluoromethyl)phenyl)urea |
| 117 | 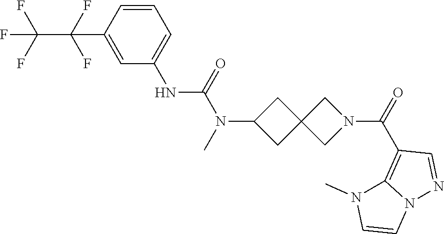 [SMILES: CN(C(=O)Nc1cccc(C(F)(F)C(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(3- (perfluoroethyl)phenyl) urea |
| 118 | 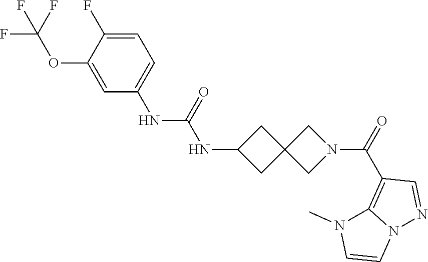 [SMILES: Cn1ccn2ncc(C(=O)N3CC4(CC(NC(=O)Nc5ccc(F)c(OC(F)(F)F)c5)C4)C3)c12] | 1-(4-fluoro-3- (trifluoromethoxy)phenyl)-3- (2-(1-methyl-1H-imidazo[1,2- b]pyrazole-7-carbonyl)-2- azaspiro[3.3]heptan-6-yl)urea |
| 119 | 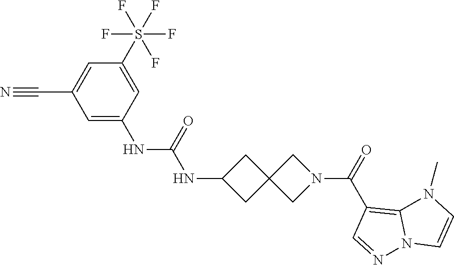 [SMILES: Cn1ccn2ncc(C(=O)N3CC4(CC(NC(=O)Nc5cc(C#N)cc(S(F)(F)(F)(F)F)c5)C4)C3)c12] | 1-(3-cyano-5-(pentafluoro-16- sulfaneyl)phenyl)-3-(2-(1- methyl-1H-imidazo[1,2- b]pyrazole-7-carbonyl)-2- azaspiro[3.3]heptan-6-yl)urea |
| 120 | 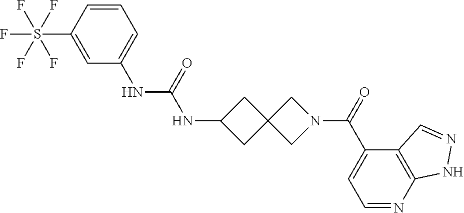 [SMILES: O=C(Nc1cccc(S(F)(F)(F)(F)F)c1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(3- (pentafluoro-16- sulfaneyl)phenyl)urea |
| 121 | 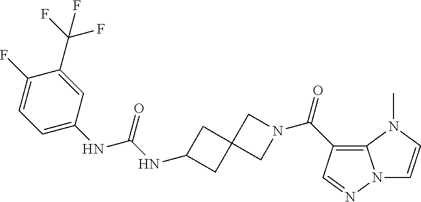 [SMILES: Cn1ccn2ncc(C(=O)N3CC4(CC(NC(=O)Nc5ccc(F)c(C(F)(F)F)c5)C4)C3)c12] | 1-(4-fluoro-3- (trifluoromethyl)phenyl)-3-(2- (1-methyl-1H-imidazo[1,2- b]pyrazole-7-carbonyl)-2- azaspiro[3.3]heptan-6-yl)urea |
| 122 | 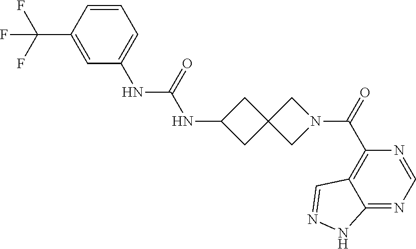 [SMILES: O=C(Nc1cccc(C(F)(F)F)c1)NC1CC2(C1)CN(C(=O)c1ncnc3[nH]ncc13)C2] | 1-(2-(1H-pyrazolo[3,4- d]pyrimidine-4-carbonyl)-2- azaspiro[3.3]heptan-6-yl)-3-(3- (trifluoromethyl)phenyl)urea |
| 123 | 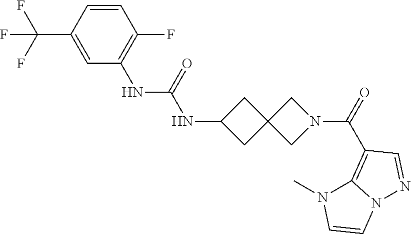 [SMILES: Cn1ccn2ncc(C(=O)N3CC4(CC(NC(=O)Nc5cc(C(F)(F)F)ccc5F)C4)C3)c12] | 1-(2-fluoro-5- (trifluoromethyl)phenyl)-3-(2- (1-methyl-1H-imidazo[1,2- b]pyrazole-7-carbonyl)-2- azaspiro[3.3]heptan-6-yl)urea |
| 124 | 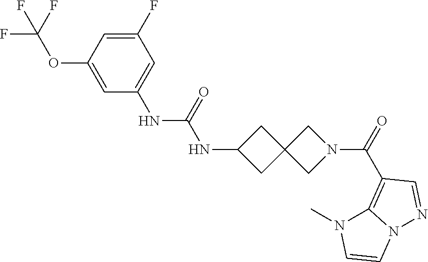 [SMILES: Cn1ccn2ncc(C(=O)N3CC4(CC(NC(=O)Nc5cc(F)cc(OC(F)(F)F)c5)C4)C3)c12] | 1-(3-fluoro-5- (trifluoromethoxy)phenyl)-3- (2-(1-methyl-1H-imidazo[1,2- b]pyrazole-7-carbonyl)-2- azaspiro[3.3]heptan-6-yl)urea |
| 125 | 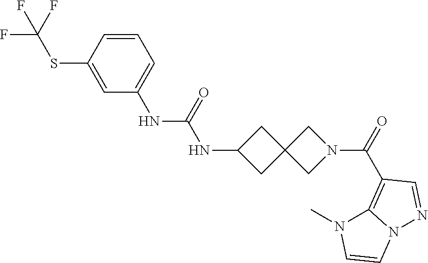 [SMILES: Cn1ccn2ncc(C(=O)N3CC4(CC(NC(=O)Nc5cccc(SC(F)(F)F)c5)C4)C3)c12] | 1-(2-(1-methyl-1H- imidazo[1,2-b]pyrazole-7- carbonyl)-2- azaspiro[3.3]heptan-6-yl)-3-(3- ((trifluoromethyl)thio)phenyl) urea |
| 126 | 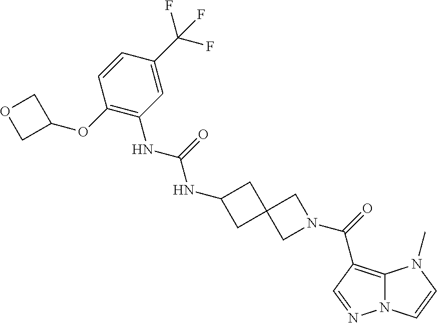 [SMILES: Cn1ccn2ncc(C(=O)N3CC4(CC(NC(=O)Nc5cc(C(F)(F)F)ccc5OC5COC5)C4)C3)c12] | 1-(2-(1-methyl-1H- imidazo[1,2-b]pyrazole-7- carbonyl)-2- azaspiro[3.3]heptan-6-yl)-3-(2- (oxetan-3-yloxy)-5- (trifluoromethyl)phenyl)urea |
| 127 | 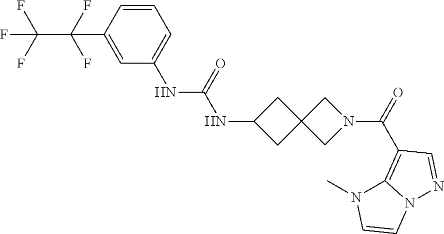 [SMILES: Cn1ccn2ncc(C(=O)N3CC4(CC(NC(=O)Nc5cccc(C(F)(F)C(F)(F)F)c5)C4)C3)c12] | 1-(2-(1-methyl-1H- imidazo[1,2-b]pyrazole-7- carbonyl)-2- azaspiro[3.3]heptan-6-yl)-3-(3- (perfluoroethyl)phenyl)urea |
| 128 | 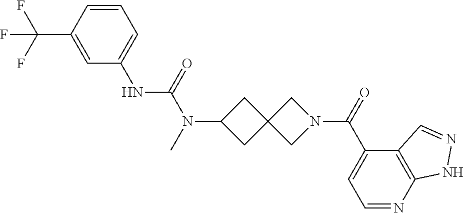 [SMILES: CN(C(=O)Nc1cccc(C(F)(F)F)c1)C1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-1- methyl-3-(3- (trifluoromethyl)phenyl)urea |
| 129 | 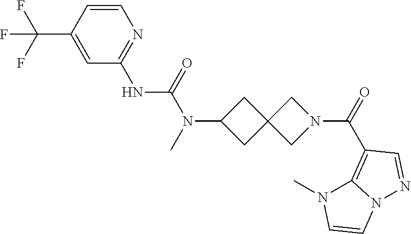 | 1-methyl-1-(2-(1-methyl-1H- imidazo[1,2-b]pyrazole-7- carbonyl)-2- azaspiro[3.3]heptan-6-yl)-3-(4- (trifluoromethyl)pyridin-2- yl)urea |
| 130 | 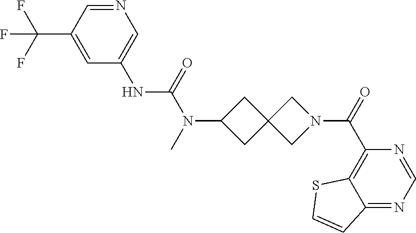 | 1-methyl-1-(2-(thieno[3,2- d]pyrimidine-4-carbonyl)-2- azaspiro[3.3]heptan-6-yl)-3-(5- (trifluoromethyl)pyridin-3- yl)urea |
| 131 | 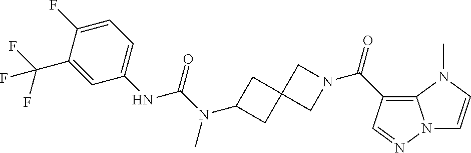 | 3-(4-fluoro-3- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 132 | 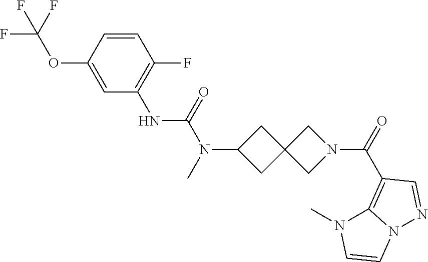 [SMILES: CN(C(=O)Nc1cc(OC(F)(F)F)ccc1F)C1CC2(C1)CN(C(=O)c1cnn3ccn(C)c13)C2] | 3-(2-fluoro-5- (trifluoromethoxy)phenyl)-1- methyl-1-(2-(1-methyl-1H- imidazo[1,2-b]pyrazole-7- carbonyl)-2- azaspiro[3.3]heptan-6-yl)urea |
| 133 | 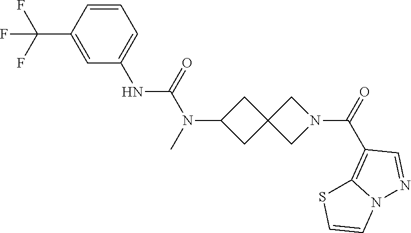 [SMILES: CN(C(=O)Nc1cccc(C(F)(F)F)c1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(3- (trifluoromethyl)phenyl)urea |
| 134 | 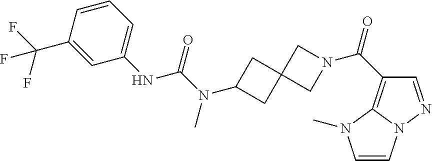 | 1-methyl-1-(2-(1-methyl-1H- imidazo[1,2-b]pyrazole-7- carbonyl)-2- azaspiro[3.3]heptan-6-yl)-3-(3- (trifluoromethyl)phenyl)urea |
| 135 | 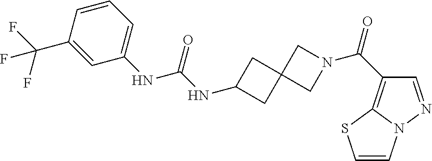 | 1-(2-(2,3-dihydropyrazolo[5,1- b]thiazole-7-carbonyl)-2- azaspiro[3.3]heptan-6-yl)-3-(3- (trifluoromethyl)phenyl)urea |
| 136 | 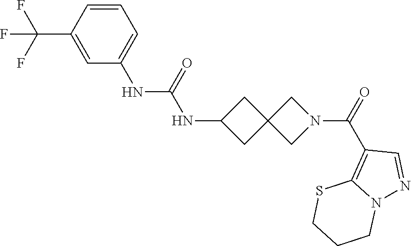 [SMILES: O=C(Nc1cccc(C(F)(F)F)c1)NC1CC2(C1)CN(C(=O)c1cnn3c1SCCC3)C2] | 1-(2-(6,7-dihydro-5H- pyrazolo[5,1-b][1,3]thiazine-3- carbonyl)-2- azaspiro[3.3]heptan-6-yl)-3-(3- (trifluoromethyl)phenyl)urea |
| 137 | 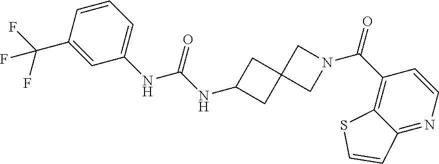 [SMILES: O=C(Nc1cccc(C(F)(F)F)c1)NC1CC2(C1)CN(C(=O)c1ccnc3ccsc13)C2] | 1-(2-(thieno[3,2-b]pyridine-7- carbonyl)-2- azaspiro[3.3]heptan-6-yl)-3-(3- (trifluoromethyl)phenyl)urea |
| 145 | 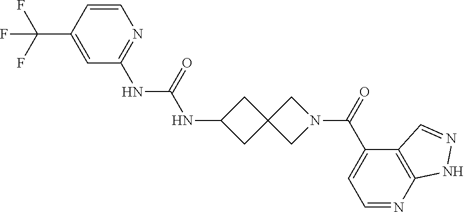 [SMILES: O=C(Nc1cc(C(F)(F)F)ccn1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(4- (trifluoromethyl)pyridin-2- yl)urea |
| 148 | 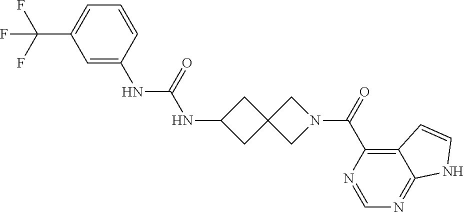 [SMILES: O=C(Nc1cccc(C(F)(F)F)c1)NC1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-3-(3- (trifluoromethyl)phenyl)urea |
| 150 | 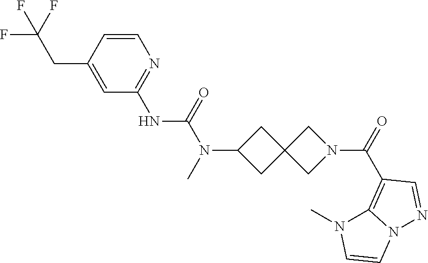 [SMILES: CN(C(=O)Nc1cc(CC(F)(F)F)ccn1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(4- (2,2,2-trifluoroethyl)pyridin-2- yl)urea |
| 151 | 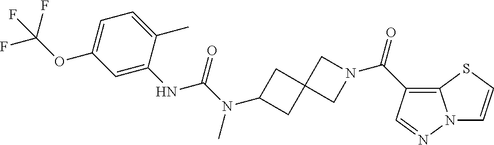 [SMILES: Cc1ccc(OC(F)(F)F)cc1NC(=O)N(C)C1CC2(C1)CN(C(=O)c1cnn3ccsc13)C2] | 1-methyl-3-(2-methyl-5- (trifluoromethoxy)phenyl)-1- (2-(pyrazolo[5,1-b]thiazole-7- carbonyl)-2- azaspiro[3.3]heptan-6-yl)urea |
| 152 | 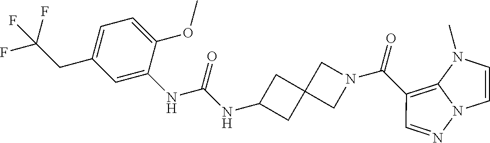 [SMILES: COc1ccc(CC(F)(F)F)cc1NC(=O)NC1CC2(C1)CN(C(=O)c1cnn3ccn(C)c13)C2] | 1-(2-methoxy-5-(2,2,2- trifluoroethyl)phenyl)-3-(2-(1- methyl-1H-imidazo[1,2- b]pyrazole-7-carbonyl)-2- azaspiro[3.3]heptan-6-yl)urea |
| 153 | 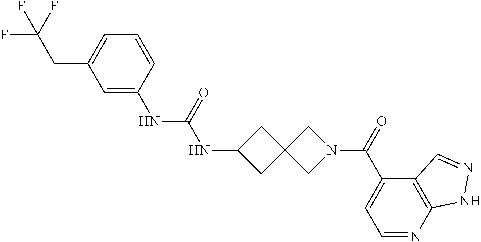 [SMILES: O=C(Nc1cccc(CC(F)(F)F)c1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(3- (2,2,2- trifluoroethyl)phenyl)urea |
| 154 | 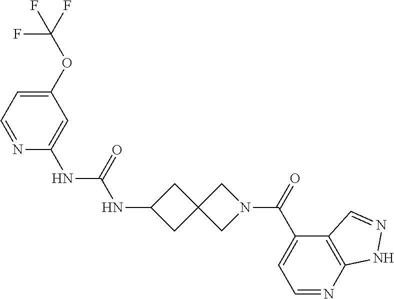 [SMILES: O=C(Nc1cc(OC(F)(F)F)ccn1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(4- (trifluoromethoxy)pyridin-2- yl)urea |
| 155 | 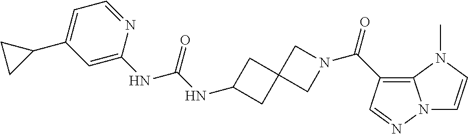 [SMILES: Cn1ccn2ncc(C(=O)N3CC4(CC(NC(=O)Nc5cc(C6CC6)ccn5)C4)C3)c12] | 1-(4-cyclopropylpyridin-2-yl)- 3-(2-(1-methyl-1H- imidazo[1,2-b]pyrazole-7- carbonyl)-2- azaspiro[3.3]heptan-6-yl)urea |
| 156 | 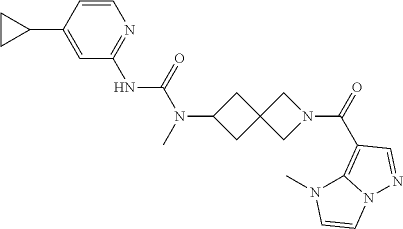 | 3-(4-cyclopropylpyridin-2-yl)- 1-methyl-1-(2-(1-methyl-1H- imidazo[1,2-b]pyrazole-7- carbonyl)-2- azaspiro[3.3]heptan-6-yl)urea |
| 157 | 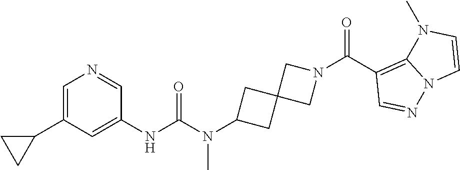 | 3-(5-cyclopropylpyridin-3-yl)- 1-methyl-1-(2-(1-methyl-1H- imidazo[1,2-b]pyrazole-7- carbonyl)-2- azaspiro[3.3]heptan-6-yl)urea |
| 158 | 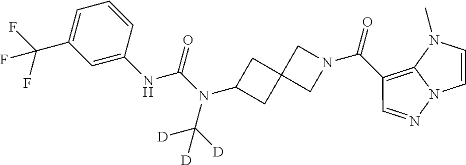 | 1-(methyl-d3)-1-(2-(1-methyl- 1H-imidazo[1,2-b]pyrazole-7- carbonyl)-2- azaspiro[3.3]heptan-6-yl)-3-(3- (trifluoromethyl)phenyl)urea |
| 159 | 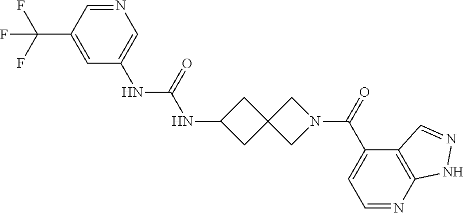 [SMILES: O=C(Nc1cncc(C(F)(F)F)c1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(5- (trifluoromethyl)pyridin-3- yl)urea |
| 160 | 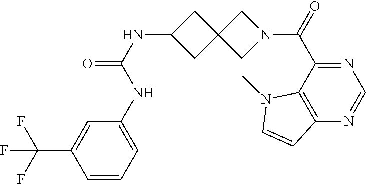 [SMILES: Cn1ccc2ncnc(C(=O)N3CC4(CC(NC(=O)Nc5cccc(C(F)(F)F)c5)C4)C3)c21] | 1-(2-(5-methyl-5H-pyrrolo[3,2- d]pyrimidine-4-carbonyl)-2- azaspiro[3.3]heptan-6-yl)-3-(3- (trifluoromethyl)phenyl)urea |
| 161 | 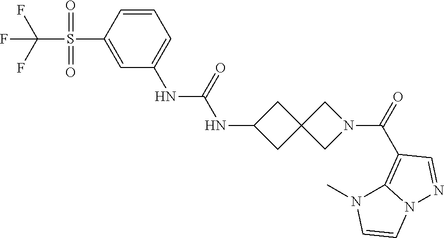 [SMILES: Cn1ccn2ncc(C(=O)N3CC4(CC(NC(=O)Nc5cccc(S(=O)(=O)C(F)(F)F)c5)C4)C3)c12] | 1-(2-(1-methyl-1H- imidazo[1,2-b]pyrazole-7- carbonyl)-2- azaspiro[3.3]heptan-6-yl)-3-(3- ((trifluoromethyl)sulfonyl) phenyl)urea |
| 162 | 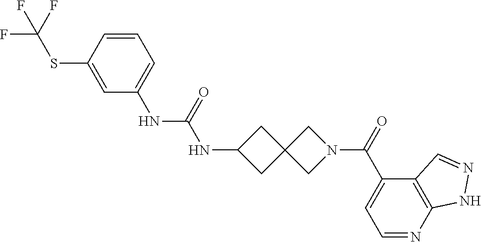 [SMILES: O=C(Nc1cccc(SC(F)(F)F)c1)NC1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-3-(3- ((trifluoromethyl)thio) phenyl)urea |
| 164 | 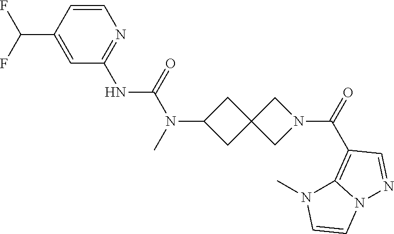 [SMILES: CN(C(=O)Nc1cc(C(F)F)ccn1)C1CC2(C1)CN(C(=O)c1cnn3ccn(C)c13)C2] | 3-(4-(difluoromethyl)pyridin- 2-yl)-1-methyl-1-(2-(1-methyl- 1H-imidazo[1,2-b]pyrazole-7- carbonyl)-2- azaspiro[3.3]heptan-6-yl)urea |
| 165 | 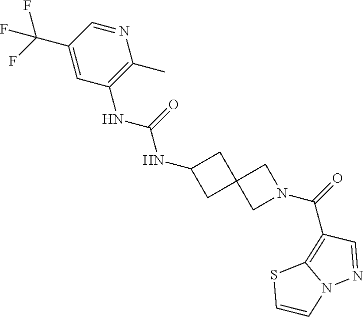 [SMILES: Cc1ncc(C(F)(F)F)cc1NC(=O)NC1CC2(C1)CN(C(=O)c1cnn3ccsc13)C2] | 1-(2-methyl-5- (trifluoromethyl)pyridin-3-yl)- 3-(2-(pyrazolo[5,1-b]thiazole- 7-carbonyl)-2- azaspiro[3.3]heptan-6-yl)urea |
| 166 | 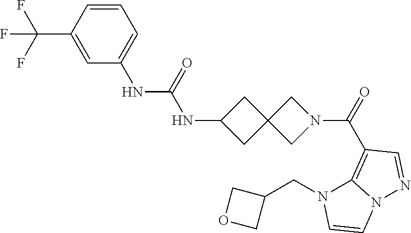 [SMILES: O=C(Nc1cccc(C(F)(F)F)c1)NC1CC2(C1)CN(C(=O)c1cnn3ccn(CC4COC4)c13)C2] | 1-(2-(1-(oxetan-3-ylmethyl)- 1H-imidazo[1,2-b]pyrazole-7- carbonyl)-2- azaspiro[3.3]heptan-6-yl)-3-(3- (trifluoromethyl)phenyl)urea |
| 167 | 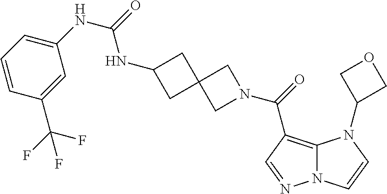 [SMILES: O=C(Nc1cccc(C(F)(F)F)c1)NC1CC2(C1)CN(C(=O)c1cnn3ccn(C4COC4)c13)C2] | 1-(2-(1-(oxetan-3-yl)-1H- imidazo[1,2-b]pyrazole-7- carbonyl)-2- azaspiro[3.3]heptan-6-yl)-3-(3- (trifluoromethyl)phenyl)urea |
| 168 | 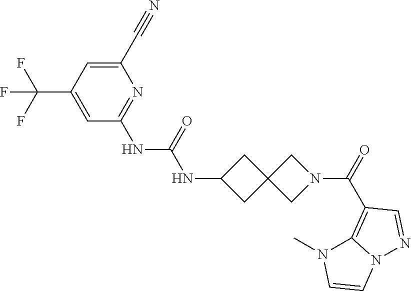 [SMILES: Cn1ccn2ncc(C(=O)N3CC4(CC(NC(=O)Nc5cc(C(F)(F)F)cc(C#N)n5)C4)C3)c12] | 1-(6-cyano-4- (trifluoromethyl)pyridin-2-yl)- 3-(2-(1-methyl-1H- imidazo[1,2-b]pyrazole-7- carbonyl)-2- azaspiro[3.3]heptan-6-yl)urea |
| 169 | 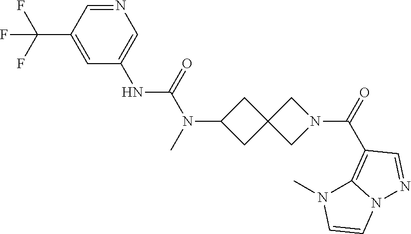 [SMILES: CN(C(=O)Nc1cncc(C(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- (trifluoromethyl)pyridin-3- yl)urea |
| 170 | 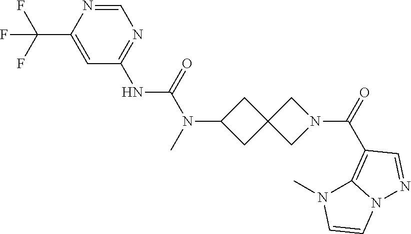 [SMILES: CN(C(=O)Nc1cc(C(F)(F)F)ncn1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(6- (trifluoromethyl)pyrimidin-4- yl)urea |
| 171 | 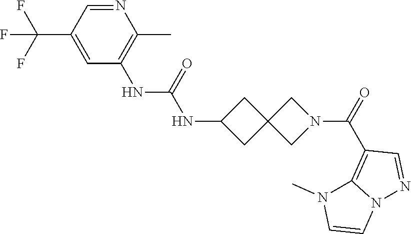 [SMILES: Cc1ncc(C(F)(F)F)cc1NC(=O)NC1CC2(C1)CN(C(=O)c1cnn3ccn(C)c13)C2] | 1-(2-(1-methyl-1H- imidazo[1,2-b]pyrazole-7- carbonyl)-2- azaspiro[3.3]heptan-6-yl)-3-(2- methyl-5- (trifluoromethyl)pyridin-3- yl)urea |
| 172 | 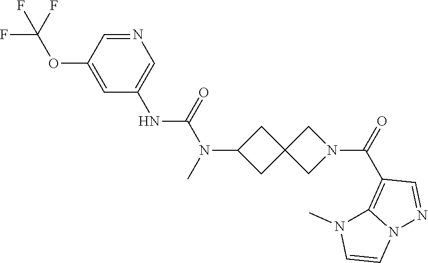 [SMILES: CN(C(=O)Nc1cncc(OC(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- (trifluoromethoxy)pyridin-3- yl)urea |
| 173 | 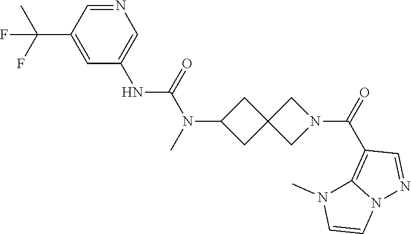 [SMILES: CN(C(=O)Nc1cncc(C(C)(F)F)c1)C1CC2(C1)CN(C(=O)c1cnn3ccn(C)c13)C2] | 3-(5-(1, 1-difluoroethyl)pyridin- 3-yl)-1-methyl-1-(2-(1-methyl- 1H-imidazo[1,2-b]pyrazole-7- carbonyl)-2- azaspiro[3.3]heptan-6-yl)urea |
| 174 | 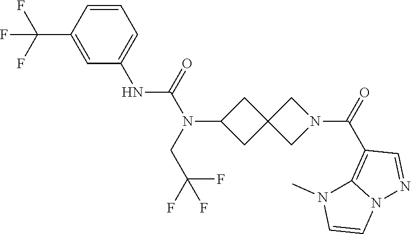 [SMILES: Cn1ccn2ncc(C(=O)N3CC4(CC(N(CC(F)(F)F)C(=O)Nc5cccc(C(F)(F)F)c5)C4)C3)c12] | 1-(2-(1-methyl-1H- imidazo[1,2-b]pyrazole-7- carbonyl)-2- azaspiro[3.3]heptan-6-yl)-1- (2,2,2-trifluoroethyl)-3-(3- (trifluoromethyl)phenyl)urea |
| 175 | 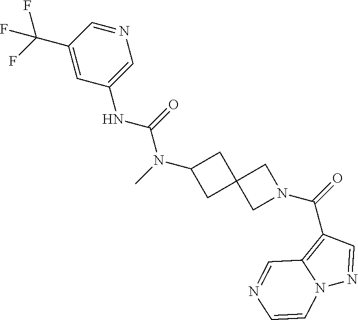 [SMILES: CN(C(=O)Nc1cncc(C(F)(F)F)c1)C1CC2(C1)CN(C(=O)c1cnn3ccncc13)C2] | 1-methyl-1-(2-(pyrazolo[1,5- a]pyrazine-3-carbonyl)-2- azaspiro[3.3]heptan-6-yl)-3-(5- (trifluoromethyl)pyridin-3- yl)urea |
| 176 | 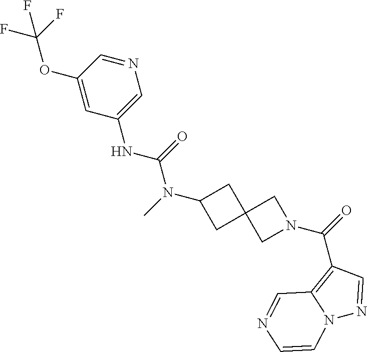 [SMILES: CN(C(=O)Nc1cncc(OC(F)(F)F)c1)C1CC2(C1)CN(C(=O)c1cnn3ccncc13)C2] | 1-methyl-1-(2-(pyrazolo[1,5- a]pyrazine-3-carbonyl)-2- azaspiro[3.3]heptan-6-yl)-3-(5- (trifluoromethoxy)pyridin-3- yl)urea |
| 177 | 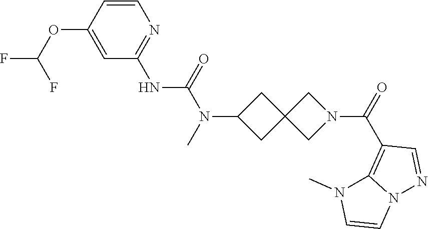 [SMILES: CN(C(=O)Nc1cc(OC(F)F)ccn1)C1CC2(C1)CN(C(=O)c1cnn3ccn(C)c13)C2] | 3-(4-(difluoromethoxy)pyridin- 2-yl)-1-methyl-1-(2-(1-methyl- 1H-imidazo[1,2-b]pyrazole-7- carbonyl)-2- azaspiro[3.3]heptan-6-yl)urea |
| 178 | 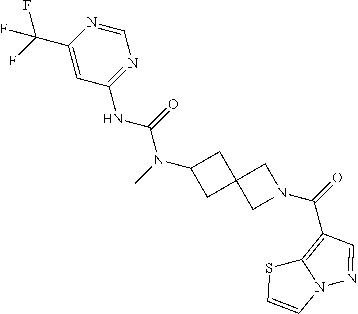 [SMILES: CN(C(=O)Nc1cc(C(F)(F)F)ncn1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(6- (trifluoromethyl)pyrimidin-4- yl)urea |
| 179 | 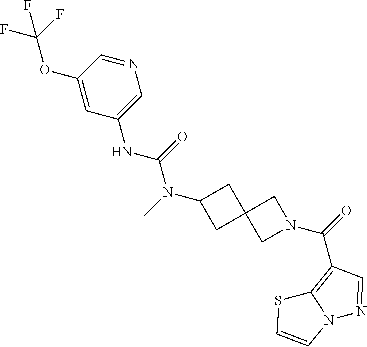 [SMILES: CN(C(=O)Nc1cncc(OC(F)(F)F)c1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(5- (trifluoromethoxy)pyridin-3- yl)urea |
| 180 | 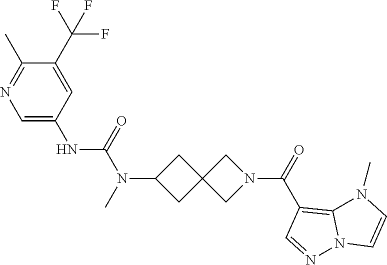 [SMILES: Cc1ncc(NC(=O)N(C)C2CC3(C2)CN(C(=O)c2cnn4ccn(C)c24)C3)cc1C(F)(F)F] | 1-methyl-1-(2-(1-methyl-1H- imidazo[1,2-b]pyrazole-7- carbonyl)-2- azaspiro[3.3]heptan-6-yl)-3-(6- methyl-5- (trifluoromethyl)pyridin-3- yl)urea |
| 181 | 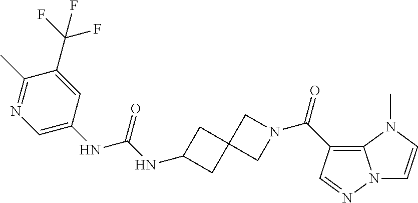 [SMILES: Cc1ncc(NC(=O)NC2CC3(C2)CN(C(=O)c2cnn4ccn(C)c24)C3)cc1C(F)(F)F] | 1-(2-(1-methyl-1H- imidazo[1,2-b]pyrazole-7- carbonyl)-2- azaspiro[3.3]heptan-6-yl)-3-(6- methyl-5- (trifluoromethyl)pyridin-3- yl)urea |
| 182 | 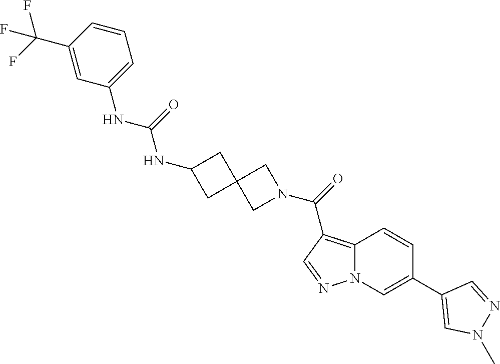 [SMILES: Cn1cc(-c2ccc3c(C(=O)N4CC5(CC(NC(=O)Nc6cccc(C(F)(F)F)c6)C5)C4)cnn3c2)cn1] | 1-(2-(6-(1-methyl-1H-pyrazol- 4-yl)pyrazolo[1,5-a]pyridine-3- carbonyl)-2- azaspiro[3.3]heptan-6-yl)-3-(3- (trifluoromethyl)phenyl)urea |
| 183 | 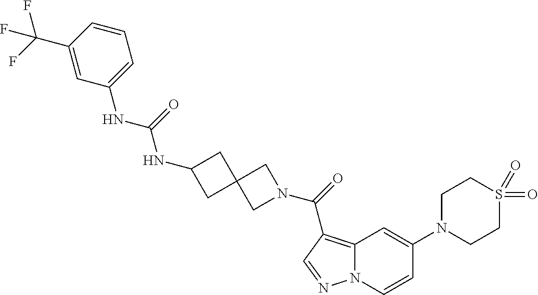 [SMILES: O=C(Nc1cccc(C(F)(F)F)c1)NC1CC2(C1)CN(C(=O)c1cnn3ccc(N4CCS(=O)(=O)CC4)cc13)C2] | 1-(2-(5-(1,1- dioxidothiomorpholino) pyrazolo[1,5-a]pyridine- 3-carbonyl)-2- azaspiro[3.3]heptan-6-yl)-3-(3- (trifluoromethyl)phenyl)urea |
| 184 | 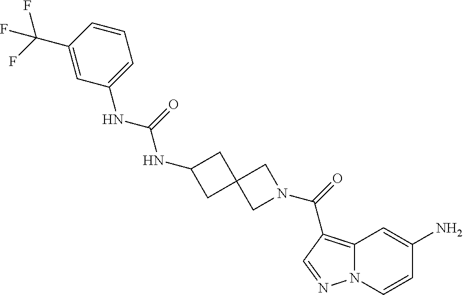 [SMILES: Nc1ccn2ncc(C(=O)N3CC4(CC(NC(=O)Nc5cccc(C(F)(F)F)c5)C4)C3)c2c1] | 1-(2-(5-aminopyrazolo[1,5- a]pyridine-3-carbonyl)-2- azaspiro[3.3]heptan-6-yl)-3-(3- (trifluoromethyl)phenyl)urea |
| 185 | 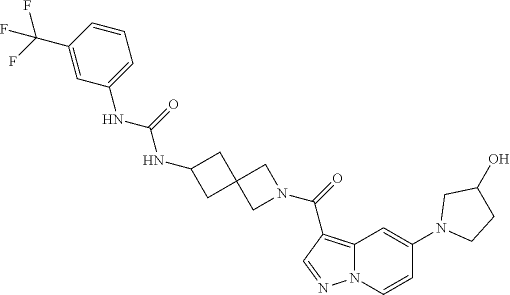 [SMILES: O=C(Nc1cccc(C(F)(F)F)c1)NC1CC2(C1)CN(C(=O)c1cnn3ccc(N4CCC(O)C4)cc13)C2] | 1-(2-(5-(3-hydroxypyrrolidin- 1-yl)pyrazolo[1,5-a]pyridine-3- carbonyl)-2- azaspiro[3.3]heptan-6-yl)-3-(3- (trifluoromethyl)phenyl)urea |
| 186 | 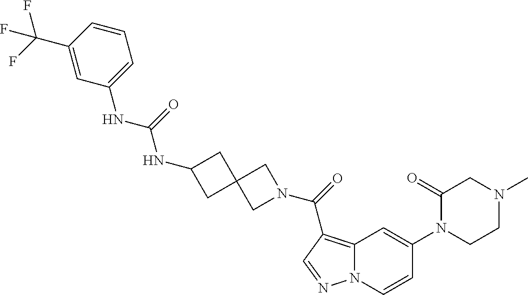 [SMILES: CN1CCN(c2ccn3ncc(C(=O)N4CC5(CC(NC(=O)Nc6cccc(C(F)(F)F)c6)C5)C4)c3c2)C(=O)C1] | 1-(2-(5-(4-methyl-2- oxopiperazin-1- yl)pyrazolo[1,5-a]pyridine-3- carbonyl)-2- azaspiro[3.3]heptan-6-yl)-3-(3- (trifluoromethyl)phenyl)urea |
| 187 | 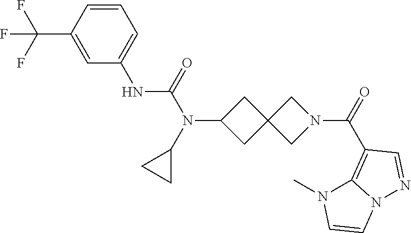 [SMILES: Cn1ccn2ncc(C(=O)N3CC4(CC(N(C(=O)Nc5cccc(C(F)(F)F)c5)C5CC5)C4)C3)c12] | 1-cyclopropyl-1-(2-(1-methyl- 1H-imidazo[1,2-b]pyrazole-7- carbonyl)-2- azaspiro[3.3]heptan-6-yl)-3-(3- (trifluoromethyl)phenyl)urea |
| 188 | 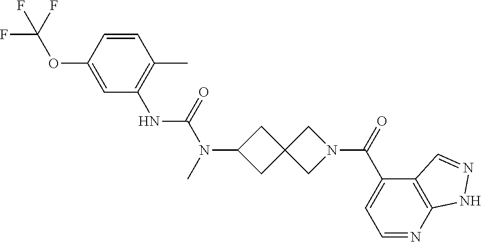 [SMILES: Cc1ccc(OC(F)(F)F)cc1NC(=O)N(C)C1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-1- methyl-3-(2-methyl-5- (trifluoromethoxy)phenyl)urea |
| 189 | 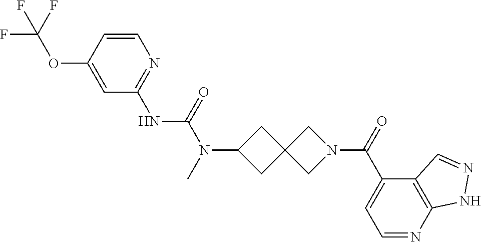 [SMILES: CN(C(=O)Nc1cc(OC(F)(F)F)ccn1)C1CC2(C1)CN(C(=O)c1ccnc3[nH]ncc13)C2] | 1-(2-(1H-pyrazolo[3,4- b]pyridine-4-carbonyl)-2- azaspiro[3.3]heptan-6-yl)-1- methyl-3-(4- (trifluoromethoxy)pyridin-2- yl)urea |
| 190 | 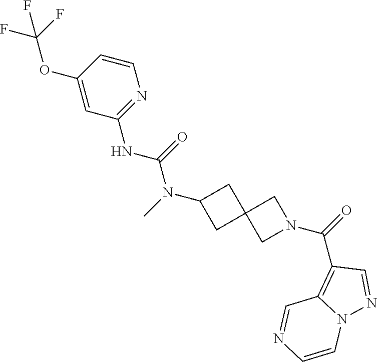 [SMILES: CN(C(=O)Nc1cc(OC(F)(F)F)ccn1)C1CC2(C1)CN(C(=O)c1cnn3ccncc13)C2] | 1-methyl-1-(2-(pyrazolo[1,5- a]pyrazine-3-carbonyl)-2- azaspiro[3.3]heptan-6-yl)-3-(4- (trifluoromethoxy)pyridin-2- yl)urea |
| 192 | 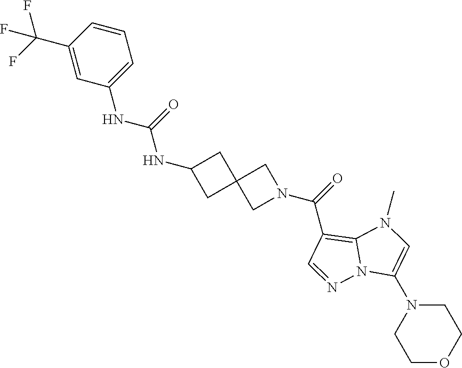 [SMILES: Cn1cc(N2CCOCC2)n2ncc(C(=O)N3CC4(CC(NC(=O)Nc5cccc(C(F)(F)F)c5)C4)C3)c12] | 1-(2-(1-methyl-3-morpholino- 1H-imidazo[1,2-b]pyrazole-7- carbonyl)-2- azaspiro[3.3]heptan-6-yl)-3-(3- (trifluoromethyl)phenyl)urea |
| 191 | 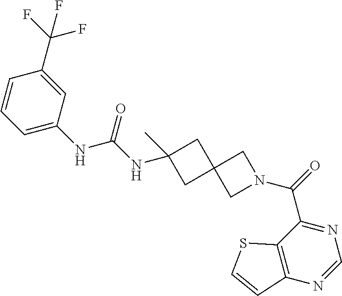 [SMILES: CC1(NC(=O)Nc2cccc(C(F)(F)F)c2)CC2(CN(C(=O)c3ncnc4ccsc34)C2)C1] | d]pyrimidine-4-carbonyl)-2- azaspiro[3.3]heptan-6-yl)-3-(3- (trifluoromethyl)phenyl)urea |
| 193 | 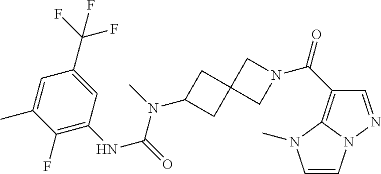 [SMILES: Cc1cc(C(F)(F)F)cc(NC(=O)N(C)C2CC3(C2)CN(C(=O)c2cnn4ccn(C)c24)C3)c1F] | 3-(2-fluoro-3-methyl-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 194 | 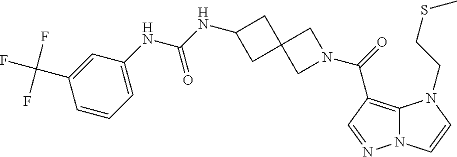 [SMILES: CSCCn1ccn2ncc(C(=O)N3CC4(CC(NC(=O)Nc5cccc(C(F)(F)F)c5)C4)C3)c12] | 1-(2-(1-(2-(methylthio)ethyl)- 1H-imidazo[1,2-b]pyrazole-7- carbonyl)-2- azaspiro[3.3]heptan-6-yl)-3-(3- (trifluoromethyl)phenyl)urea |
| 195 | 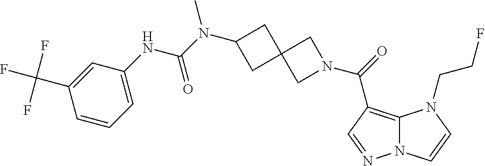 [SMILES: CN(C(=O)Nc1cccc(C(F)(F)F)c1)C1CC2(C1)CN(C(=O)c1cnn3ccn(CCF)c13)C2] | 1-(2-(1-(2-fluoroethyl)-1H- imidazo[1,2-b]pyrazole-7- carbonyl)-2- azaspiro[3.3]heptan-6-yl)-1- methyl-3-(3- (trifluoromethyl)phenyl)urea |
| 196 | 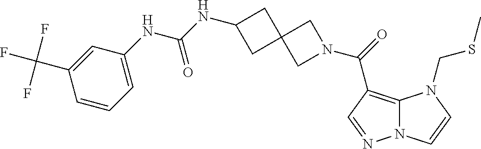 [SMILES: CSCn1ccn2ncc(C(=O)N3CC4(CC(NC(=O)Nc5cccc(C(F)(F)F)c5)C4)C3)c12] | 1-(2-(1-((methylthio)methyl)- 1H-imidazo[1,2-b]pyrazole-7- carbonyl)-2- azaspiro[3.3]heptan-6-yl)-3-(3- (trifluoromethyl)phenyl)urea |
| 197 | 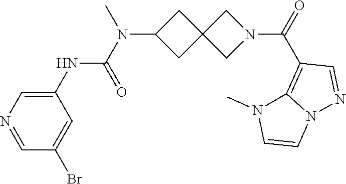 [SMILES: CN(C(=O)Nc1cncc(Br)c1)C1CC2(C1)CN(C(=O)c1cnn3ccn(C)c13)C2] | 3-(5-bromopyridin-3-yl)-1- methyl-1-(2-(1-methyl-1H- imidazo[1,2-b]pyrazole-7- carbonyl)-2- azaspiro[3.3]heptan-6-yl)urea |
| 198 | 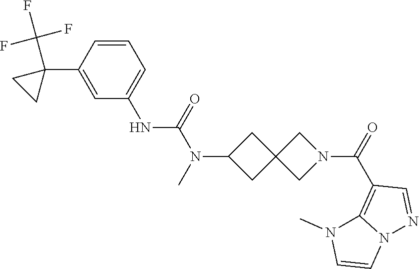 [SMILES: CN(C(=O)Nc1cccc(C2(C(F)(F)F)CC2)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(3- (1-(trifluoromethyl) cyclopropyl)phenyl)urea |
| 199 | 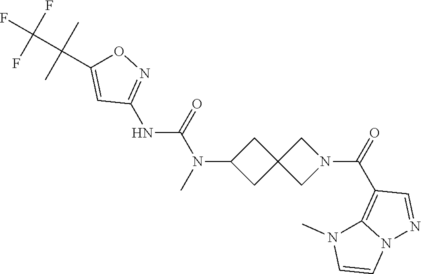 [SMILES: CN(C(=O)Nc1cc(C(C)(C)C(F)(F)F)on1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- (1,1,1-trifluoro-2- methylpropan-2-yl)isoxazol-3- yl)urea |
| 200 | 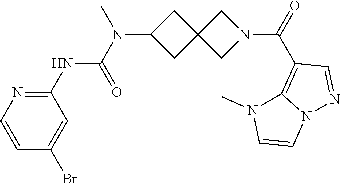 [SMILES: CN(C(=O)Nc1cc(Br)ccn1)C1CC2(C1)CN(C(=O)c1cnn3ccn(C)c13)C2] | 3-(4-bromopyridin-2-yl)-1- methyl-1-(2-(1-methyl-1H- imidazo[1,2-b]pyrazole-7- carbonyl)-2- azaspiro[3.3]heptan-6-yl)urea |
| 201 | 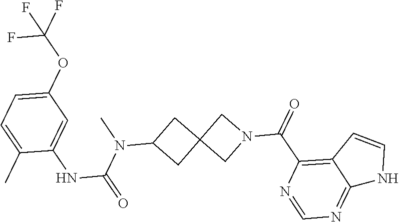 [SMILES: Cc1ccc(OC(F)(F)F)cc1NC(=O)N(C)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-1- methyl-3-(2-methyl-5- (trifluoromethoxy)phenyl)urea |
| 202 | 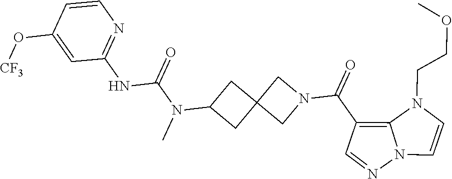 [SMILES: COCCn1ccn2ncc(C(=O)N3CC4(CC(N(C)C(=O)Nc5cc(OC(F)(F)F)ccn5)C4)C3)c12] | 1-(2-(1-(2-methoxyethyl)-1H- imidazo[1,2-b]pyrazole-7- carbonyl)-2- azaspiro[3.3]heptan-6-yl)-1- methyl-3-(4- (trifluoromethoxy)pyridin-2- yl)urea |
| 203 | 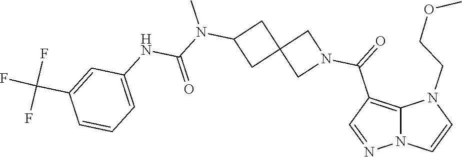 [SMILES: COCCn1ccn2ncc(C(=O)N3CC4(CC(N(C)C(=O)Nc5cccc(C(F)(F)F)c5)C4)C3)c12] | 1-(2-(1-(2-methoxyethyl)-1H- imidazo[1,2-b]pyrazole-7- carbonyl)-2- azaspiro[3.3]heptan-6-yl)-1- methyl-3-(3- (trifluoromethyl)phenyl)urea |
| 204 | 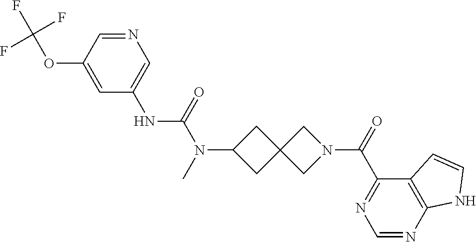 [SMILES: CN(C(=O)Nc1cncc(OC(F)(F)F)c1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-1- methyl-3-(5- (trifluoromethoxy)pyridin-3- yl)urea |
| 205 | 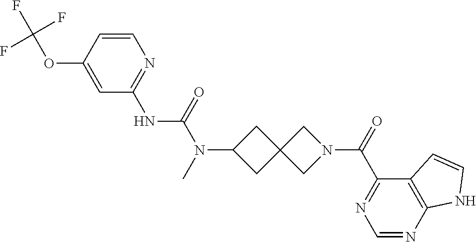 [SMILES: CN(C(=O)Nc1cc(OC(F)(F)F)ccn1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-1- methyl-3-(4- (trifluoromethoxy)pyridin-2- yl)urea |
| 206 | 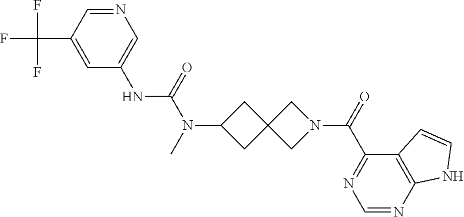 [SMILES: CN(C(=O)Nc1cncc(C(F)(F)F)c1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-1- methyl-3-(5- (trifluoromethyl)pyridin-3- yl)urea |
| 207 | 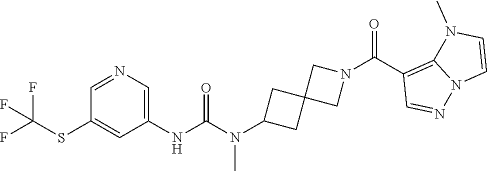 [SMILES: CN(C(=O)Nc1cncc(SC(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- ((trifluoromethyl)thio)pyridin- 3-yl)urea |
| 208 | 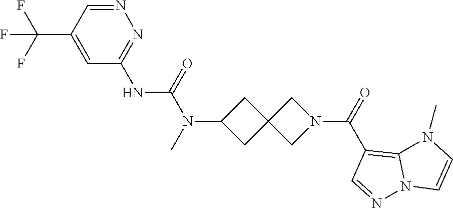 [SMILES: CN(C(=O)Nc1cc(C(F)(F)F)cnn1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- (trifluoromethyl)pyridazin-3- yl)urea |
| 209 | 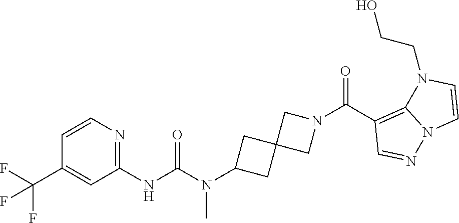 [SMILES: CN(C(=O)Nc1cc(C(F)(F)F)ccn1)C1CC2(C1)CN(C(=O)c1cnn3ccn(CCO)c13)C2] | 1-(2-(1-(2-hydroxyethyl)-1H- imidazo[1,2-b]pyrazole-7- carbonyl)-2- azaspiro[3.3]heptan-6-yl)-1- methyl-3-(4- (trifluoromethyl)pyridin-2- yl)urea |
| 210 | 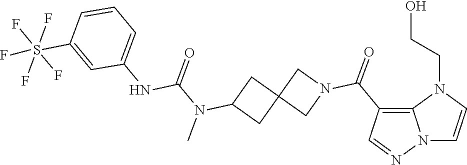 [SMILES: CN(C(=O)Nc1cccc(S(F)(F)(F)(F)F)c1)C1CC2(C1)CN(C(=O)c1cnn3ccn(CCO)c13)C2] | 1-(2-(1-(2-hydroxyethyl)-1H- imidazo[1,2-b]pyrazole-7- carbonyl)-2- azaspiro[3.3]heptan-6-yl)-1- methyl-3-(3-(pentafluoro-16- sulfaneyl)phenyl)urea |
| 211 | 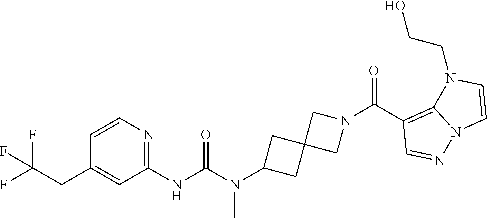 [SMILES: CN(C(=O)Nc1cc(CC(F)(F)F)ccn1)C1CC2(C1)CN(C(=O)c1cnn3ccn(CCO)c13)C2] | 1-(2-(1-(2-hydroxyethyl)-1H- imidazo[1,2-b]pyrazole-7- carbonyl)-2- azaspiro[3.3]heptan-6-yl)-1- methyl-3-(4- (trifluoromethoxy)pyridin-2- yl)urea |
| 212 | 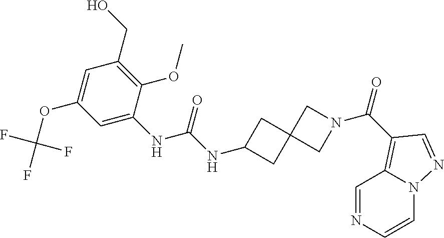 [SMILES: COc1c(CO)cc(OC(F)(F)F)cc1NC(=O)NC1CC2(C1)CN(C(=O)c1cnn3ccncc13)C2] | 1-(3-(hydroxymethyl)-2- methoxy-5- (trifluoromethoxy)phenyl)-3- (2-(pyrazolo[1,5-a]pyrazine-3- carbonyl)-2- azaspiro[3.3]heptan-6-yl)urea |
| 213 | 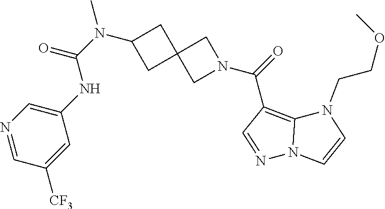 [SMILES: COCCn1ccn2ncc(C(=O)N3CC4(CC(N(C)C(=O)Nc5cncc(C(F)(F)F)c5)C4)C3)c12] | 1-(2-(1-(2-methoxyethyl)-1H- imidazo[1,2-b]pyrazole-7- carbonyl)-2- azaspiro[3.3]heptan-6-yl)-1- methyl-3-(5- (trifluoromethyl)pyridin-3- yl)urea |
| 214 | 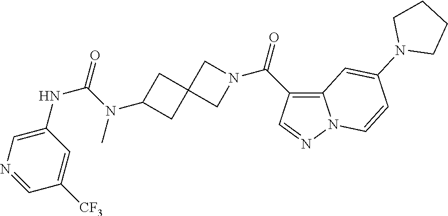 [SMILES: CN(C(=O)Nc1cncc(C(F)(F)F)c1)C1CC2(C1)CN(C(=O)c1cnn3ccc(N4CCCC4)cc13)C2] | 1-methyl-1-(2-(5-(pyrrolidin-1- yl)pyrazolo[1,5-a]pyridine-3- carbonyl)-2- azaspiro[3.3]heptan-6-yl)-3-(5- (trifluoromethyl)pyridin-3- yl)urea |
| 215 | 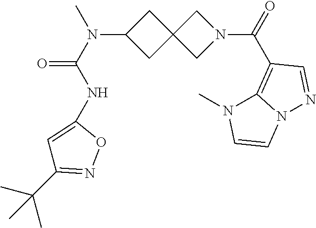 [SMILES: CN(C(=O)Nc1cc(C(C)(C)C)no1)C1CC2(C1)CN(C(=O)c1cnn3ccn(C)c13)C2] | 3-(3-(tert-butyl)isoxazol-5-yl)- 1-methyl-1-(2-(1-methyl-1H- imidazo[1,2-b]pyrazole-7- carbonyl)-2- azaspiro[3.3]heptan-6-yl)urea |
| 216 | 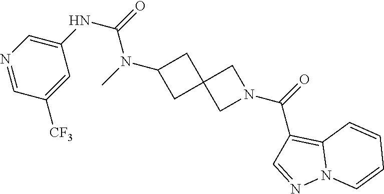 [SMILES: CN(C(=O)Nc1cncc(C(F)(F)F)c1)C1CC2(C1)CN(C(=O)c1cnn3ccccc13)C2] | 1-methyl-1-(2-(pyrazolo[1,5- a]pyridine-3-carbonyl)-2- azaspiro[3.3]heptan-6-yl)-3-(5- (trifluoromethyl)pyridin-3- yl)urea |
| 217 | 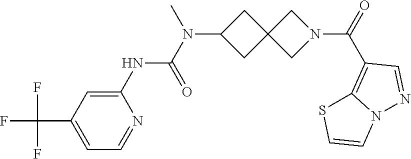 [SMILES: CN(C(=O)Nc1cc(C(F)(F)F)ccn1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(4- (trifluoromethyl)pyridin-2- yl)urea |
| 222 | 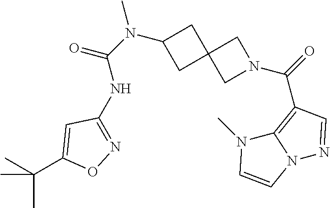 [SMILES: CN(C(=O)Nc1cc(C(C)(C)C)on1)C1CC2(C1)CN(C(=O)c1cnn3ccn(C)c13)C2] | 3-(5-(tert-butyl)isoxazol-3-yl)- 1-methyl-1-(2-(1-methyl-1H- imidazo[1,2-b]pyrazole-7- carbonyl)-2- azaspiro[3.3]heptan-6-yl)urea |
| 223 | 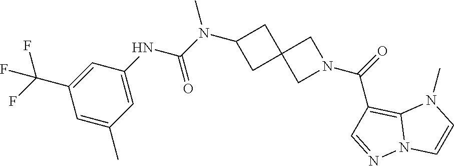 [SMILES: Cc1cc(NC(=O)N(C)C2CC3(C2)CN(C(=O)c2cnn4ccn(C)c24)C3)cc(C(F)(F)F)c1] | 1-methyl-1-(2-(1-methyl-1H- imidazo[1,2-b]pyrazole-7- carbonyl)-2- azaspiro[3.3]heptan-6-yl)-3-(3- methyl-5- (trifluoromethyl)phenyl)urea |
| 224 | 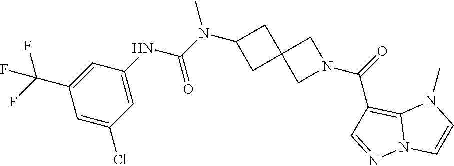 [SMILES: CN(C(=O)Nc1cc(Cl)cc(C(F)(F)F)c1)C1CC2(C1)CN(C(=O)c1cnn3ccn(C)c13)C2] | 3-(3-chloro-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 225 | 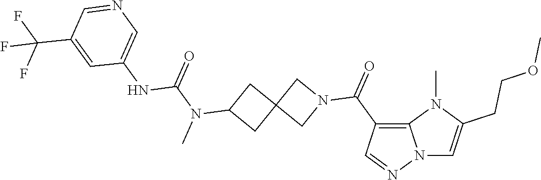 [SMILES: COCCc1cn2ncc(C(=O)N3CC4(CC(N(C)C(=O)Nc5cncc(C(F)(F)F)c5)C4)C3)c2n1C] | 1-(2-(2-(2-methoxyethyl)-1- methyl-1H-imidazo[1,2- b]pyrazole-7-carbonyl)-2- azaspiro[3.3]heptan-6-yl)-1- methyl-3-(5- (trifluoromethyl)pyridin-3- yl)urea |
| 226 | 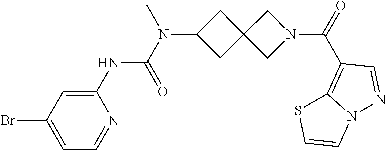 [SMILES: CN(C(=O)Nc1cc(Br)ccn1)C1CC2(C1)CN(C(=O)c1cnn3ccsc13)C2] | 3-(4-bromopyridin-2-yl)-1- methyl-1-(2-(pyrazolo[5,1- b]thiazole-7-carbonyl)-2- azaspiro[3.3]heptan-6-yl)urea |
| 227 | 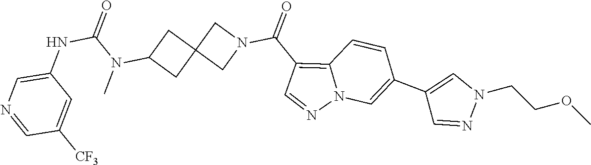 [SMILES: COCCn1cc(-c2ccc3c(C(=O)N4CC5(CC(N(C)C(=O)Nc6cncc(C(F)(F)F)c6)C5)C4)cnn3c2)cn1] | 1-(2-(6-(1-(2-methoxyethyl)- 1H-pyrazol-4-yl)pyrazolo[1,5- a]pyridine-3-carbonyl)-2- azaspiro[3.3]heptan-6-yl)-1- methyl-3-(5- (trifluoromethyl)pyridin-3- yl)urea |
| 228 | 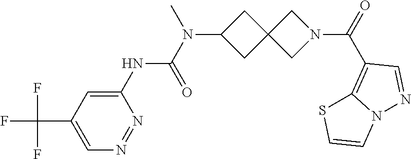 [SMILES: CN(C(=O)Nc1cc(C(F)(F)F)cnn1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(5- (trifluoromethyl)pyridazin-3- yl)urea |
| 229 | 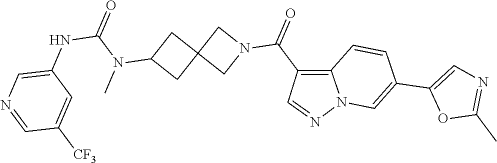 [SMILES: Cc1ncc(-c2ccc3c(C(=O)N4CC5(CC(N(C)C(=O)Nc6cncc(C(F)(F)F)c6)C5)C4)cnn3c2)o1] | 1-methyl-1-(2-(6-(2- methyloxazol-5- yl)pyrazolo[1,5-a]pyridine-3- carbonyl)-2- azaspiro[3.3]heptan-6-yl)-3-(5- (trifluoromethyl)pyridin-3- yl)urea |
| 230 | 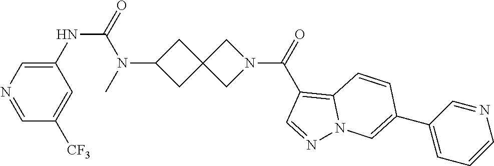 [SMILES: CN(C(=O)Nc1cncc(C(F)(F)F)c1)C1CC2(C1)CN(C(=O)c1cnn3cc(-c4cccnc4)ccc13)C2] | 1-methyl-1-(2-(6-(pyridin-3- yl)pyrazolo[1,5-a]pyridine-3- carbonyl)-2- azaspiro[3.3]heptan-6-yl)-3-(5- (trifluoromethyl)pyridin-3- yl)urea |
| 231 | 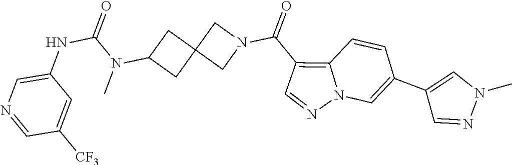 [SMILES: CN(C(=O)Nc1cncc(C(F)(F)F)c1)C1CC2(C1)CN(C(=O)c1cnn3cc(-c4cnn(C)c4)ccc13)C2] | 1-methyl-1-(2-(6-(1-methyl- 1H-pyrazol-4-yl)pyrazolo[1,5- a]pyridine-3-carbonyl)-2- azaspiro[3.3]heptan-6-yl)-3-(5- (trifluoromethyl)pyridin-3- yl)urea |
| 232 | 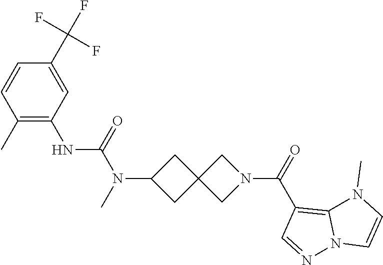 [SMILES: Cc1ccc(C(F)(F)F)cc1NC(=O)N(C)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(2- methyl-5- (trifluoromethyl)phenyl)urea |
| 233 | 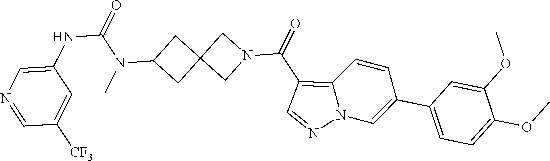 [SMILES: COc1ccc(-c2ccc3c(C(=O)N4CC5(CC(N(C)C(=O)Nc6cncc(C(F)(F)F)c6)C5)C4)cnn3c2)cc1OC] | 1-(2-(6-(3,4- dimethoxyphenyl)pyrazolo [1,5-a]pyridine-3-carbonyl)-2- azaspiro[3.3]heptan-6-yl)-1- methyl-3-(5- (trifluoromethyl)pyridin-3- yl)urea |
| 234 | 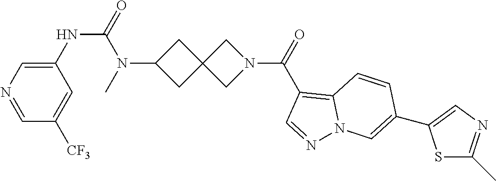 [SMILES: Cc1ncc(-c2ccc3c(C(=O)N4CC5(CC(N(C)C(=O)Nc6cncc(C(F)(F)F)c6)C5)C4)cnn3c2)s1] | 1-methyl-1-(2-(6-(2- methylthiazol-5- yl)pyrazolo[1,5-a]pyridine-3- carbonyl)-2- azaspiro[3.3]heptan-6-yl)-3-(5- (trifluoromethyl)pyridin-3- yl)urea |
| 235 | 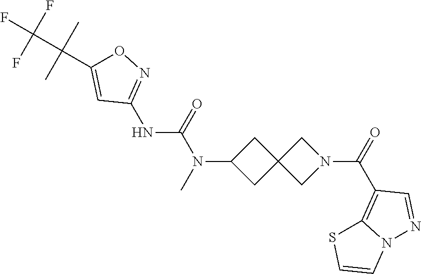 [SMILES: CN(C(=O)Nc1cc(C(C)(C)C(F)(F)F)on1)C1CC2(C1)CN(C(=O)c1cnn3ccsc13)C2] | 1-methyl-1-(2-(pyrazolo[5,1- b]thiazole-7-carbonyl)-2- azaspiro[3.3]heptan-6-yl)-3-(5- (1, 1,1-trifluoro-2- methylpropan-2-yl)isoxazol-3- yl)urea |
| 236 | 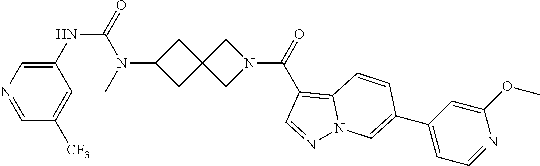 [SMILES: COc1cc(-c2ccc3c(C(=O)N4CC5(CC(N(C)C(=O)Nc6cncc(C(F)(F)F)c6)C5)C4)cnn3c2)ccn1] | 1-(2-(6-(2-methoxypyridin-4- yl)pyrazolo[1,5-a]pyridine-3- carbonyl)-2- azaspiro[3.3]heptan-6-yl)-1- methyl-3-(5- (trifluoromethyl)pyridin-3- yl)urea |
| 237 | 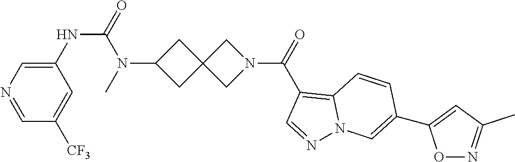 [SMILES: Cc1cc(-c2ccc3c(C(=O)N4CC5(CC(N(C)C(=O)Nc6cncc(C(F)(F)F)c6)C5)C4)cnn3c2)on1] | 1-methyl-1-(2-(6-(3- methylisoxazol-5- yl)pyrazolo[1,5-a]pyridine-3- carbonyl)-2- azaspiro[3.3]heptan-6-yl)-3-(5- (trifluoromethyl)pyridin-3- yl)urea |
| 238 | 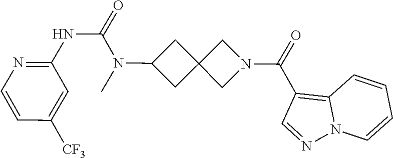 [SMILES: CN(C(=O)Nc1cc(C(F)(F)F)ccn1)C1CC2(C1)CN(C(=O)c1cnn3ccccc13)C2] | 1-methyl-1-(2-(pyrazolo[1,5- a]pyridine-3-carbonyl)-2- azaspiro[3.3]heptan-6-yl)-3-(4- (trifluoromethyl)pyridin-2- yl)urea |
| 239 | 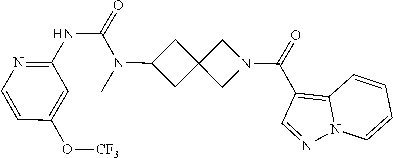 [SMILES: CN(C(=O)Nc1cc(OC(F)(F)F)ccn1)C1CC2(C1)CN(C(=O)c1cnn3ccccc13)C2] | 1-methyl-1-(2-(pyrazolo[1,5- a]pyridine-3-carbonyl)-2- azaspiro[3.3]heptan-6-yl)-3-(4- (trifluoromethoxy)pyridin-2- yl)urea |
| 240 | 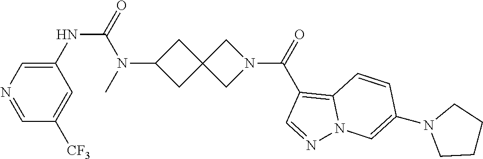 [SMILES: CN(C(=O)Nc1cncc(C(F)(F)F)c1)C1CC2(C1)CN(C(=O)c1cnn3cc(N4CCCC4)ccc13)C2] | 1-methyl-1-(2-(6-(pyrrolidin-1- yl)pyrazolo[1,5-a]pyridine-3- carbonyl)-2- azaspiro[3.3]heptan-6-yl)-3-(5- (trifluoromethyl)pyridin-3- yl)urea |
| 241 | 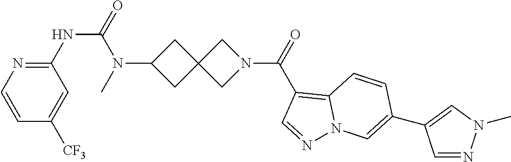 [SMILES: CN(C(=O)Nc1cc(C(F)(F)F)ccn1)C1CC2(C1)CN(C(=O)c1cnn3cc(-c4cnn(C)c4)ccc13)C2] | 1-methyl-1-(2-(6-(1-methyl- 1H-pyrazol-4-yl)pyrazolo[1,5- a]pyridine-3-carbonyl)-2- azaspiro[3.3]heptan-6-yl)-3-(4- (trifluoromethyl)pyridin-2- yl)urea |
| 242 | 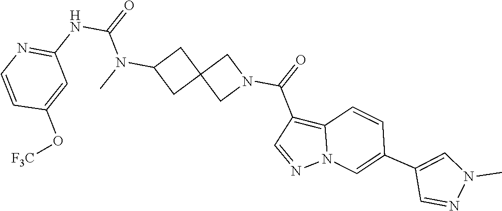 [SMILES: CN(C(=O)Nc1cc(OC(F)(F)F)ccn1)C1CC2(C1)CN(C(=O)c1cnn3cc(-c4cnn(C)c4)ccc13)C2] | 1-(2-(6-(1,2-dimethyl-1H-214- pyrazol-4-yl)pyrazolo[1,5- a]pyridine-3-carbonyl)-2- azaspiro[3.3]heptan-6-yl)-1- methyl-3-(4- (trifluoromethoxy)pyridin-2- yl)urea |
| 243 | 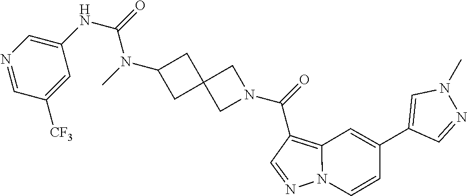 [SMILES: CN(C(=O)Nc1cncc(C(F)(F)F)c1)C1CC2(C1)CN(C(=O)c1cnn3ccc(-c4cnn(C)c4)cc13)C2] | 1-methyl-1-(2-(5-(1-methyl- 1H-pyrazol-4-yl)pyrazolo[1,5- a]pyridine-3-carbonyl)-2- azaspiro[3.3]heptan-6-yl)-3- (5-(trifluoromethyl)pyridin-3- yl)urea |
| 244 | 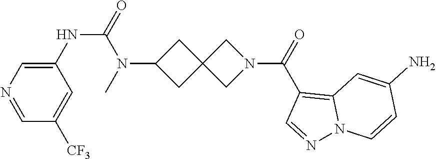 [SMILES: CN(C(=O)Nc1cncc(C(F)(F)F)c1)C1CC2(C1)CN(C(=O)c1cnn3ccc(N)cc13)C2] | 1-(2-(5-aminopyrazolo[1,5- a]pyridine-3-carbonyl)-2- azaspiro[3.3]heptan-6-yl)-1- methyl-3-(5- (trifluoromethyl)pyridin-3- yl)urea |
| 245 | 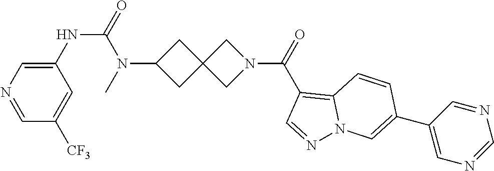 [SMILES: CN(C(=O)Nc1cncc(C(F)(F)F)c1)C1CC2(C1)CN(C(=O)c1cnn3cc(-c4cncnc4)ccc13)C2] | 1-methyl-1-(2-(6-(pyrimidin- 5-yl)pyrazolo[1,5-a]pyridine- 3-carbonyl)-2- azaspiro[3.3]heptan-6-yl)-3- (5-(trifluoromethyl)pyridin-3- yl)urea |
| 246 | 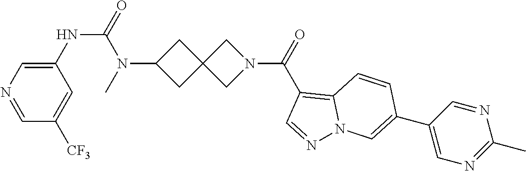 [SMILES: Cc1ncc(-c2ccc3c(C(=O)N4CC5(CC(N(C)C(=O)Nc6cncc(C(F)(F)F)c6)C5)C4)cnn3c2)cn1] | 1-methyl-1-(2-(6-(2- methylpyrimidin-5- yl)pyrazolo[1,5-a]pyridine-3- carbonyl)-2- azaspiro[3.3]heptan-6-yl)-3- (5-(trifluoromethyl)pyridin- 3-yl)urea |
| 247 | 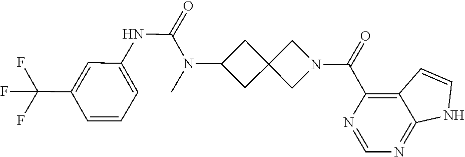 [SMILES: CN(C(=O)Nc1cccc(C(F)(F)F)c1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)-1- methyl-3-(3- (trifluoromethyl)phenyl)urea |
| 248 | 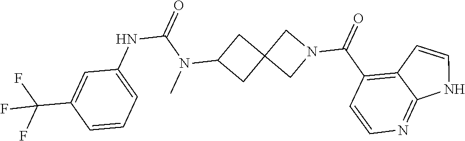 [SMILES: CN(C(=O)Nc1cccc(C(F)(F)F)c1)C1CC2(C1)CN(C(=O)c1ccnc3[nH]ccc13)C2] | 1-(2-(1H-pyrrolo[2,3- b]pyridine-4-carbonyl)-2- azaspiro[3.3]heptan-6-yl)-1- methyl-3-(3- (trifluoromethyl)phenyl)urea |
| 249 | 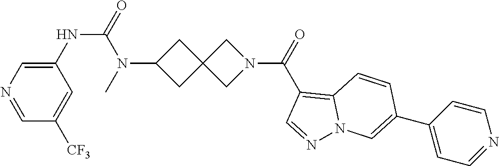 [SMILES: CN(C(=O)Nc1cncc(C(F)(F)F)c1)C1CC2(C1)CN(C(=O)c1cnn3cc(-c4ccncc4)ccc13)C2] | 1-methyl-1-(2-(6-(pyridin-4- yl)pyrazolo[1,5-a]pyridine-3- carbonyl)-2- azaspiro[3.3]heptan-6-yl)-3- (5-(trifluoromethyl)pyridin- 3-yl)urea |
| 250 | 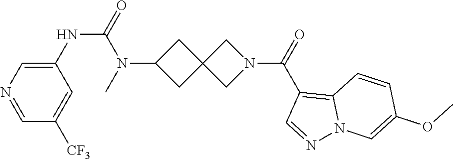 [SMILES: COc1ccc2c(C(=O)N3CC4(CC(N(C)C(=O)Nc5cncc(C(F)(F)F)c5)C4)C3)cnn2c1] | 1-(2-(6-methoxypyrazolo[1,5- a]pyridine-3-carbonyl)-2- azaspiro[3.3]heptan-6-yl)-1- methyl-3-(5- (trifluoromethyl)pyridin-3- yl)urea |
| 251 | 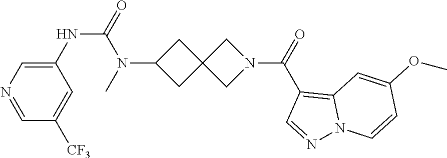 [SMILES: COc1ccn2ncc(C(=O)N3CC4(CC(N(C)C(=O)Nc5cncc(C(F)(F)F)c5)C4)C3)c2c1] | 1-(2-(5-methoxypyrazolo[1,5- a]pyridine-3-carbonyl)-2- azaspiro[3.3]heptan-6-yl)-1- methyl-3-(5- (trifluoromethyl)pyridin-3- yl)urea |
| 252 | 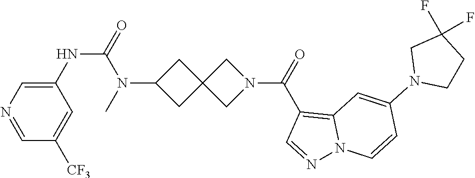 [SMILES: CN(C(=O)Nc1cncc(C(F)(F)F)c1)C1CC2(C1)CN(C(=O)c1cnn3ccc(N4CCC(F)(F)C4)cc13)C2] | 1-(2-(5-(3,3-difluoropyrrolidin- 1-yl)pyrazolo[1,5-a]pyridine- 3-carbonyl)-2- azaspiro[3.3]heptan-6-yl)-1- methyl-3-(5- (trifluoromethyl)pyridin-3- yl)urea |
| 253 | 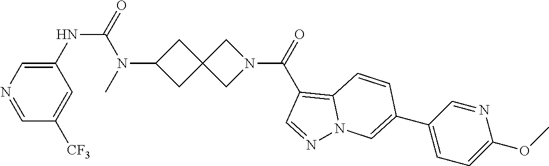 [SMILES: COc1ccc(-c2ccc3c(C(=O)N4CC5(CC(N(C)C(=O)Nc6cncc(C(F)(F)F)c6)C5)C4)cnn3c2)cn1] | 1-(2-(6-(6-methoxypyridin-3- yl)pyrazolo[1,5-a]pyridine-3- carbonyl)-2- azaspiro[3.3]heptan-6-yl)-1- methyl-3-(5- (trifluoromethyl)pyridin-3- yl)urea |
| 254 | 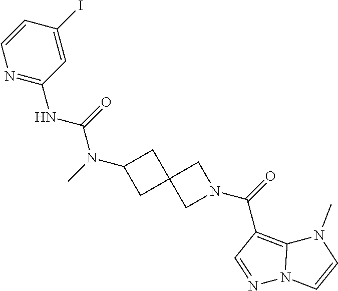 [SMILES: CN(C(=O)Nc1cc(I)ccn1)C1CC2(C1)CN(C(=O)c1cnn3ccn(C)c13)C2] | 3-(4-iodopyridin-2-yl)-1- methyl-1-(2-(1-methyl-1H- imidazo[1,2-b]pyrazole-7- carbonyl)-2- azaspiro[3.3]heptan-6-yl)urea |
| 255 | 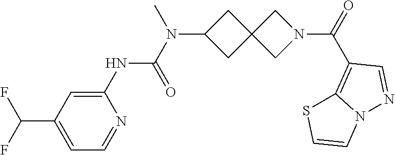 [SMILES: CN(C(=O)Nc1cc(C(F)F)ccn1)C1CC2(C1)CN(C(=O)c1cnn3ccsc13)C2] | 3-(4-(difluoromethyl)pyridin- 2-yl)-1-methyl-1-(2- (pyrazolo[5,1-b]thiazole-7- carbonyl)-2- azaspiro[3.3]heptan-6-yl)urea |
| 256 | 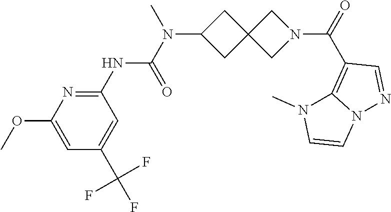 [SMILES: COc1cc(C(F)(F)F)cc(NC(=O)N(C)C2CC3(C2)CN(C(=O)c2cnn4ccn(C)c24)C3)n1] | 3-(6-methoxy-4- (trifluoromethyl)pyridin-2-yl)- 1-methyl-1-(2-(1-methyl-1H- imidazo[1,2-b]pyrazole-7- carbonyl)-2- azaspiro[3.3]heptan-6-yl)urea |
| 257 | 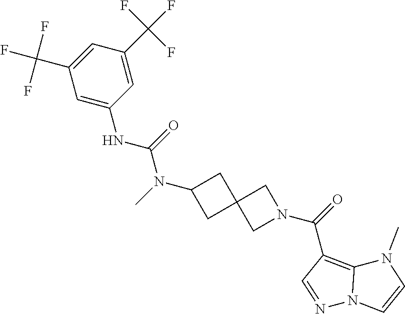 [SMILES: CN(C(=O)Nc1cc(C(F)(F)F)cc(C(F)(F)F)c1)C1CC2(C1)CN(C(=O)c1cnn3ccn(C)c13)C2] | 3-(3,5- bis(trifluoromethyl)phenyl)-1- methyl-1-(2-(1-methyl-1H- imidazo[1,2-b]pyrazole-7- carbonyl)-2- azaspiro[3.3]heptan-6-yl)urea |
| 258 | 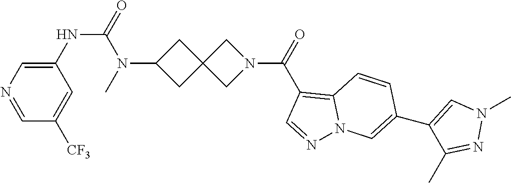 [SMILES: Cc1nn(C)cc1-c1ccc2c(C(=O)N3CC4(CC(N(C)C(=O)Nc5cncc(C(F)(F)F)c5)C4)C3)cnn2c1] | 1-(2-(6-(1,3-dimethyl-1H- pyrazol-4-yl)pyrazolo[1,5- a]pyridine-3-carbonyl)-2- azaspiro[3.3]heptan-6-yl)-1- methyl-3-(5- (trifluoromethyl)pyridin-3- yl)urea |
| 259 | 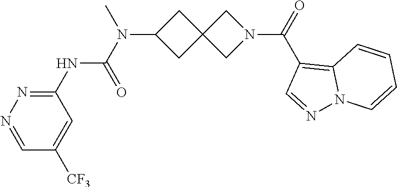 [SMILES: CN(C(=O)Nc1cc(C(F)(F)F)cnn1)C1CC2(C1)CN(C(=O)c1cnn3ccccc13)C2] | 1-methyl-1-(2-(pyrazolo[1,5- a]pyridine-3-carbonyl)-2- azaspiro[3.3]heptan- 6-yl)-3-(5- (trifluoromethyl)pyridazin- 3-yl)urea |
| 260 | 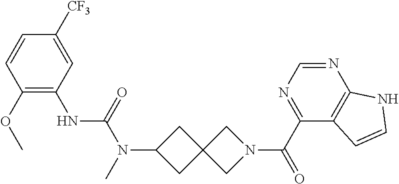 [SMILES: COc1ccc(C(F)(F)F)cc1NC(=O)N(C)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6-yl)- 3-(2-methoxy-5- (trifluoromethyl)phenyl)-1- methylurea |
| 261 | 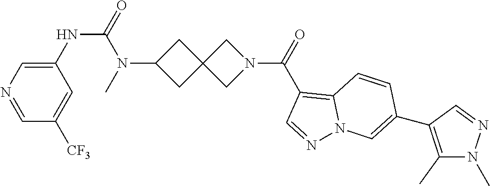 [SMILES: Cc1c(-c2ccc3c(C(=O)N4CC5(CC(N(C)C(=O)Nc6cncc(C(F)(F)F)c6)C5)C4)cnn3c2)cnn1C] | 1-(2-(6-(1,5-dimethyl-1H- pyrazol-4-yl)pyrazolo[1,5- a]pyridine-3-carbonyl)-2- azaspiro[3.3]heptan-6-yl)-1- methyl-3-(5- (trifluoromethyl)pyridin-3- yl)urea |
| 262 | 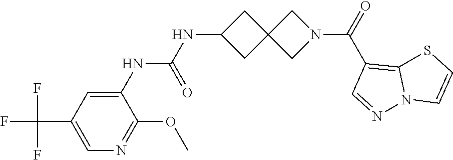 [SMILES: COc1ncc(C(F)(F)F)cc1NC(=O)NC1CC2(C1)CN(C(=O)c1cnn3ccsc13)C2] | 1-(2-methoxy-5- (trifluoromethyl)pyridin-3-yl)- 3-(2-(pyrazolo[5,1-b]thiazole- 7-carbonyl)-2- azaspiro[3.3]heptan-6-yl)urea |
| 263 | 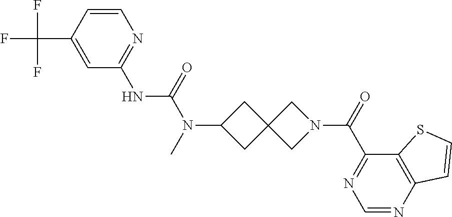 [SMILES: CN(C(=O)Nc1cc(C(F)(F)F)ccn1)C1CC2(C1)CN(C(=O)c1ncnc3ccsc13)C2] | 1-methyl-1-(2-(thieno[3,2- d]pyrimidine-4-carbonyl)-2- azaspiro[3.3]heptan-6-yl)-3- (4-(trifluoromethyl)pyridin-2- yl)urea |
| 264 | 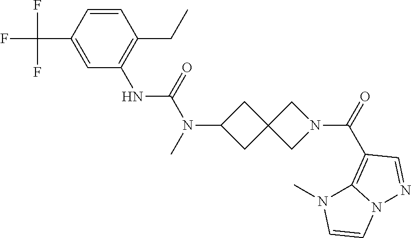 [SMILES: CCc1ccc(C(F)(F)F)cc1NC(=O)N(C)C1CC2(C1)CN(C(=O)c1cnn3ccn(C)c13)C2] | 3-(2-ethyl-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 265 | 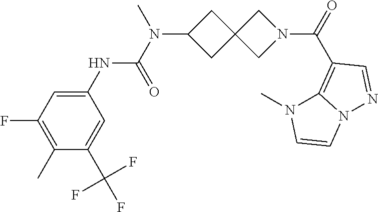 [SMILES: Cc1c(F)cc(NC(=O)N(C)C2CC3(C2)CN(C(=O)c2cnn4ccn(C)c24)C3)cc1C(F)(F)F] | 3-(3-fluoro-4-methyl-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 266 | 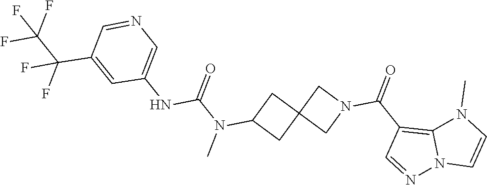 [SMILES: CN(C(=O)Nc1cncc(C(F)(F)C(F)(F)F)c1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(5- (perfluoroethyl)pyridin-3- yl)urea |
| 267 | 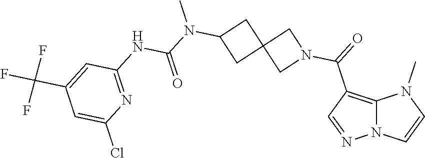 [SMILES: CN(C(=O)Nc1cc(C(F)(F)F)cc(Cl)n1)C1CC2(C1)CN(C(=O)c1cnn3ccn(C)c13)C2] | 3-(6-chloro-4- (trifluoromethyl)pyridin-2-yl)- 1-methyl-1-(2-(1-methyl-1H- imidazo[1,2-b]pyrazole-7- carbonyl)-2- azaspiro[3.3]heptan-6-yl)urea |
| 268 | 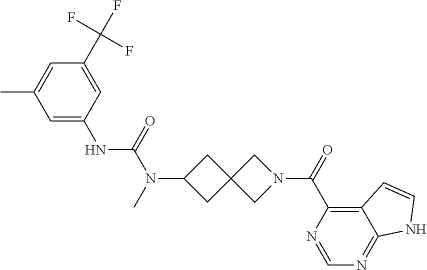 [SMILES: Cc1cc(NC(=O)N(C)C2CC3(C2)CN(C(=O)c2ncnc4[nH]ccc24)C3)cc(C(F)(F)F)c1] | 1-(2-(7H-pyrrolo[2,3- d]pyrimidine-4-carbonyl)-2- azaspiro[3.3]heptan-6-yl)-1- methyl-3-(3-methyl-5- (trifluoromethyl)phenyl)urea |
| 269 | 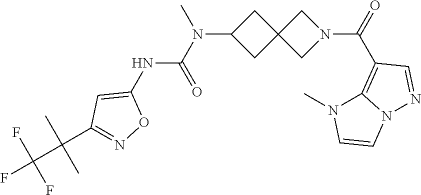 [SMILES: CN(C(=O)Nc1cc(C(C)(C)C(F)(F)F)no1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)- 3-(3-(1,1,1-trifluoro-2- methylpropan-2-yl)isoxazol-5- yl)urea |
| 280 | 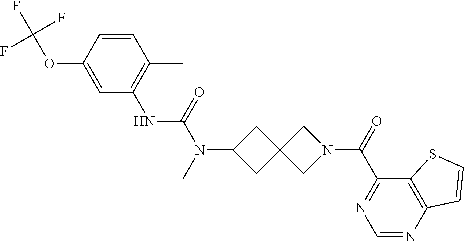 [SMILES: Cc1ccc(OC(F)(F)F)cc1NC(=O)N(C)C1CC2(C1)CN(C(=O)c1ncnc3ccsc13)C2] | 1-methyl-3-(2-methyl-5- (trifluoromethoxy)phenyl)-1- (2-(thieno[3,2-d]pyrimidine-4- carbonyl)-2- azaspiro[3.3]heptan-6-yl)urea |
| 281 | 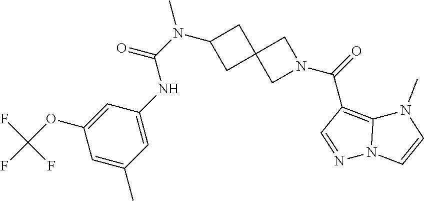 [SMILES: Cc1cc(NC(=O)N(C)C2CC3(C2)CN(C(=O)c2cnn4ccn(C)c24)C3)cc(OC(F)(F)F)c1] | 1-methyl-1-(2-(1-methyl-1H- imidazo[1,2-b]pyrazole-7- carbonyl)-2- azaspiro[3.3]heptan- 6-yl)-3-(3-methyl-5- (trifluoromethoxy)phenyl)urea |
| 282 | 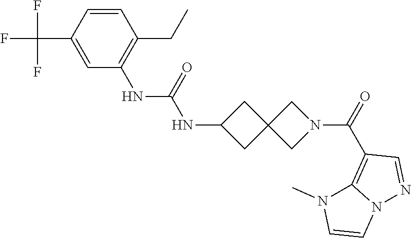 [SMILES: CCc1ccc(C(F)(F)F)cc1NC(=O)NC1CC2(C1)CN(C(=O)c1cnn3ccn(C)c13)C2] | 1-(2-ethyl-5- (trifluoromethyl)phenyl)-3-(2- (1-methyl-1H-imidazo[1,2- b]pyrazole-7-carbonyl)-2- azaspiro[3.3]heptan-6-yl)urea |
| 283 | 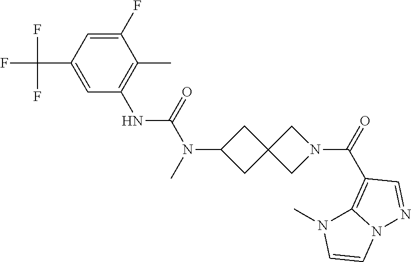 [SMILES: Cc1c(F)cc(C(F)(F)F)cc1NC(=O)N(C)C1CC2(C1)CN(C(=O)c1cnn3ccn(C)c13)C2] | 3-(3-fluoro-2-methyl-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 284 | 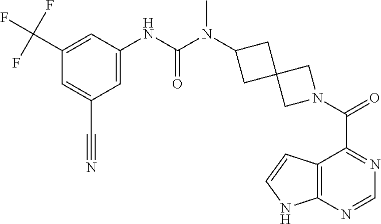 [SMILES: CN(C(=O)Nc1cc(C#N)cc(C(F)(F)F)c1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6- yl)-3-(3-cyano-5- (trifluoromethyl)phenyl)-1- methylurea |
| 285 | 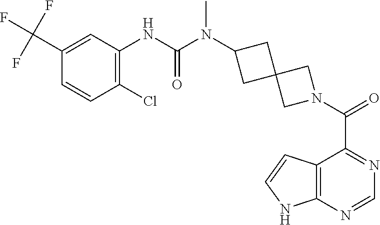 [SMILES: CN(C(=O)Nc1cc(C(F)(F)F)ccc1Cl)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6- yl)-3-(2-chloro-5- (trifluoromethyl)phenyl)-1- methylurea |
| 286 | 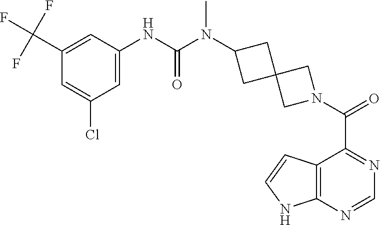 [SMILES: CN(C(=O)Nc1cc(Cl)cc(C(F)(F)F)c1)C1CC2(C1)CN(C(=O)c1ncnc3[nH]ccc13)C2] | 1-(2-(7H-pyrrolo[2,3- d]pyrimidine-4-carbonyl)-2- azaspiro[3.3]heptan-6- yl)-3-(3-chloro-5- (trifluoromethyl)phenyl)-1- methylurea |
| 287 | 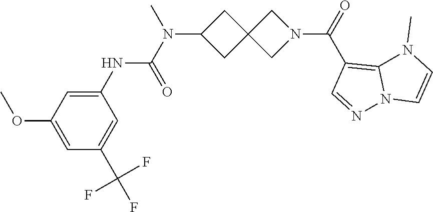 [SMILES: COc1cc(NC(=O)N(C)C2CC3(C2)CN(C(=O)c2cnn4ccn(C)c24)C3)cc(C(F)(F)F)c1] | 3-(3-methoxy-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 288 | 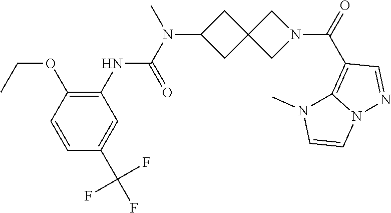 [SMILES: CCOc1ccc(C(F)(F)F)cc1NC(=O)N(C)C1CC2(C1)CN(C(=O)c1cnn3ccn(C)c13)C2] | 3-(2-ethoxy-5- (trifluoromethyl)phenyl)-1- methyl-1-(2-(1-methyl-1H- imidazo[1,2-b]pyrazole-7- carbonyl)-2- azaspiro[3.3]heptan-6-yl)urea |
| 289 | 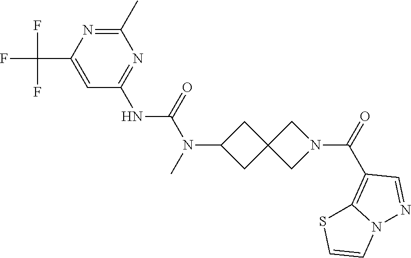 [SMILES: Cc1nc(NC(=O)N(C)C2CC3(C2)CN(C(=O)c2cnn4ccsc24)C3)cc(C(F)(F)F)n1] | 1-methyl-3-(2-methyl-6- (trifluoromethyl)pyrimidin-4- yl)-1-(2-(pyrazolo[5,1- b]thiazole-7-carbonyl)-2- azaspiro[3.3]heptan-6-yl)urea |
| 290 | 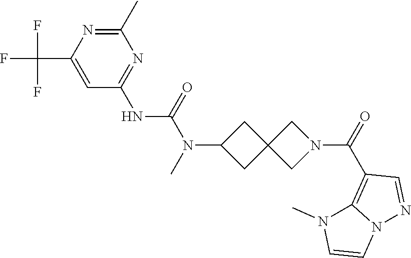 [SMILES: Cc1nc(NC(=O)N(C)C2CC3(C2)CN(C(=O)c2cnn4ccn(C)c24)C3)cc(C(F)(F)F)n1] | 1-methyl-1-(2-(1-methyl-1H- imidazo[1,2-b]pyrazole-7- carbonyl)-2- azaspiro[3.3]heptan-6- yl)-3-(2-methyl-6- (trifluoromethyl)pyrimidin- 4-yl)urea |
| 291 | 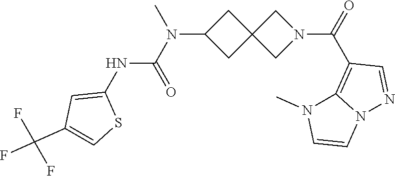 [SMILES: CN(C(=O)Nc1cc(C(F)(F)F)cs1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)-3-(4- (trifluoromethyl)thiophen-2- yl)urea |
| 298 | 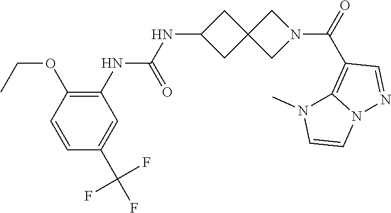 [SMILES: CCOc1ccc(C(F)(F)F)cc1NC(=O)NC1CC2(C1)CN(C(=O)c1cnn3ccn(C)c13)C2] | 1-(2-ethoxy-5- (trifluoromethyl)phenyl)-3-(2- (1-methyl-1H-imidazo[1,2- b]pyrazole-7-carbonyl)-2- azaspiro[3.3]heptan-6-yl)urea |
| 300 | 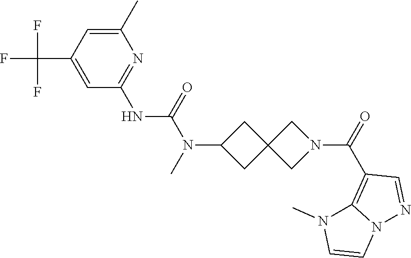 [SMILES: Cc1cc(C(F)(F)F)cc(NC(=O)N(C)C2CC3(C2)CN(C(=O)c2cnn4ccn(C)c24)C3)n1] | 1-methyl-1-(2-(1-methyl-1H- imidazo[1,2-b]pyrazole-7- carbonyl)-2- azaspiro[3.3]heptan-6-yl)- 3-(6-methyl-4- (trifluoromethyl)pyridin-2- yl)urea |
| 303 | 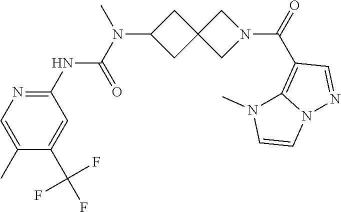 [SMILES: Cc1cnc(NC(=O)N(C)C2CC3(C2)CN(C(=O)c2cnn4ccn(C)c24)C3)cc1C(F)(F)F] | 1-methyl-1-(2-(1-methyl-1H- imidazo[1,2-b]pyrazole-7- carbonyl)-2- azaspiro[3.3]heptan-6-yl)-3-(5- methyl-4- (trifluoromethyl)pyridin-2- yl)urea |
| 305 | 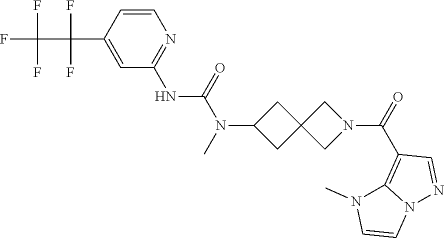 [SMILES: CN(C(=O)Nc1cc(C(F)(F)C(F)(F)F)ccn1)C1CC2(C1)CN(C(=O)c1cnn3ccn(C)c13)C2] | 1-methyl-1-(2-(1-methyl-1H- imidazo[1,2-b]pyrazole-7- carbonyl)-2- azaspiro[3.3]heptan-6-yl)- 3-(4-(perfluoroethyl) pyridin-2-yl)urea |
| 308 | 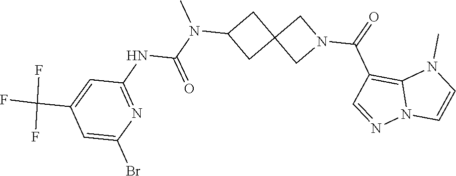 [SMILES: CN(C(=O)Nc1cc(C(F)(F)F)cc(Br)n1)C1CC2(C1)CN(C(=O)c1cnn3ccn(C)c13)C2] | 3-(6-bromo-4- (trifluoromethyl)pyridin-2-yl)- 1-methyl-1-(2-(1-methyl-1H- imidazo[1,2-b]pyrazole-7- carbonyl)-2- azaspiro[3.3]heptan-6-yl)urea |
| 309 | 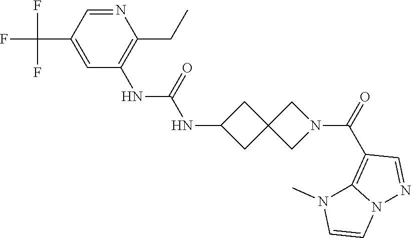 [SMILES: CCc1ncc(C(F)(F)F)cc1NC(=O)NC1CC2(C1)CN(C(=O)c1cnn3ccn(C)c13)C2] | 1-(2-ethyl-5- (trifluoromethyl)pyridin-3-yl)- 3-(2-(1-methyl-1H- imidazo[1,2-b]pyrazole-7- carbonyl)-2- azaspiro[3.3]heptan-6-yl)urea |
| 310 | 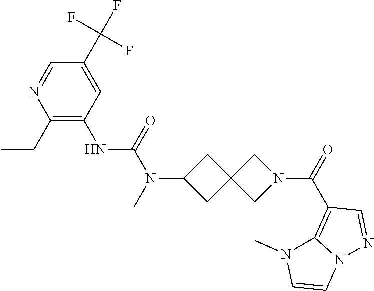 [SMILES: CCc1ncc(C(F)(F)F)cc1NC(=O)N(C)C1CC2(C1)CN(C(=O)c1cnn3ccn(C)c13)C2] | 3-(2-ethyl-5- (trifluoromethyl)pyridin-3-yl)- 1-methyl-1-(2-(1-methyl-1H- imidazo[1,2-b]pyrazole-7- carbonyl)-2- azaspiro[3.3]heptan-6-yl)urea |

Particularly preferred embodiments of the invention are the compounds of formula (Ib) as defined above, wherein A is optionally substituted phenyl, L1 is NH and B is selected from imidazo[1,2-a]pyrazinyl and 1H-pyrazolo[3,4-b]pyridinyl.

Further particularly preferred compounds of formula (Ib) are those listed in Table 2 below, and pharmaceutically acceptable salts thereof. These compounds are particularly active on receptors DDR1 and DDR2, as shown in Table 44 further below.

TABLE 2
List of representative compounds of Formula (Ib)

| Example N^{o} | Structure | Chemical Name |
|---|---|---|
| 11 | 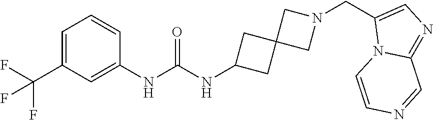 [SMILES: O=C(Nc1cccc(C(F)(F)F)c1)NC1CC2(C1)CN(Cc1cnc3cnccn13)C2] | 1-(2-(imidazo[1,2-a]pyrazin- 3-ylmethyl)-2- azaspiro[3.3]heptan-6-yl)-3- (3- (trifluoromethyl)phenyl)urea |
| 12 | 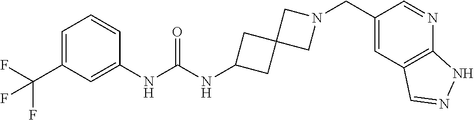 [SMILES: O=C(Nc1cccc(C(F)(F)F)c1)NC1CC2(C1)CN(Cc1cnc3[nH]ncc3c1)C2] | 1-(2-((1H-pyrazolo[3,4- b]pyridine-5-yl)methyl)-2- azaspiro[3.3]heptan-6-yl)-3- (3- (trifluoromethyl)phenyl)urea |

Additional particularly preferred embodiments of the invention are the compounds of formula (Ic) as defined above, wherein A is optionally substituted phenyl or pyridinyl and B is selected from 1H-pyrazolo[3,4-b]pyridinyl, imidazo[1,5-a]pyrazinyl and 1H-pyrrolo[2,3-b]pyridinyl, and wherein R is preferably hydrogen or CH₃.

More particularly preferred compounds of formula (Ic) are those listed in Table 3 below, and pharmaceutically acceptable salts thereof. These compounds are particularly active on receptors DDR1 and DDR2, as shown in Table 44 further below.

TABLE 3
List of representative compounds of Formula (Ic)

| Example | | |
|---|---|---|
| N^{o} | Structure | Chemical Name |
| 15 | 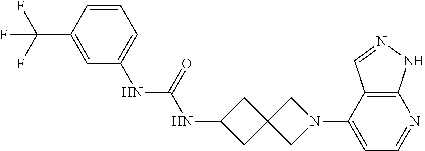 [SMILES: O=C(Nc1cccc(C(F)(F)F)c1)NC1CC2(C1)CN(c1ccnc3[nH]ncc13)C2] | 1-(2-(1H-pyrazolo[3,4- b]pyridin-4-yl)-2- azaspiro[3.3]heptan-6-yl)-3- (3- (trifluoromethyl)phenyl)urea |
| 138 | 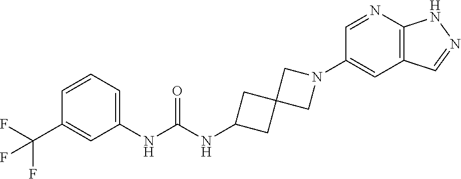 [SMILES: O=C(Nc1cccc(C(F)(F)F)c1)NC1CC2(C1)CN(c1cnc3[nH]ncc3c1)C2] | 1-(2-(1H-pyrazolo[3,4- b]pyridine-5-yl)-2- azaspiro[3.3]heptan-6-yl)-3- (3- (trifluoromethyl)phenyl)urea |
| 149 | 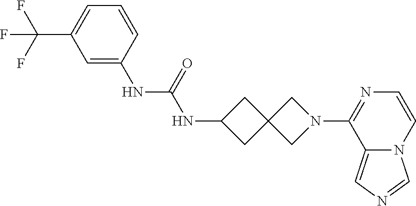 [SMILES: O=C(Nc1cccc(C(F)(F)F)c1)NC1CC2(C1)CN(c1nccn3cncc13)C2] | 1-(2-(imidazo[1,5-a]pyrazin- 8-yl)-2-azaspiro[3.3]heptan- 6-yl)-3-(3- (trifluoromethyl)phenyl)urea |
| 163 | 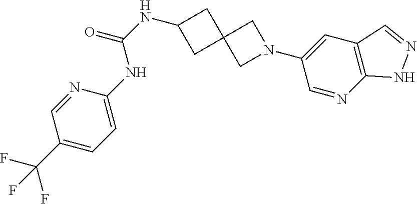 [SMILES: O=C(Nc1ccc(C(F)(F)F)cn1)NC1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (5-(trifluoromethyl)pyridin-2- yl)urea |
| 218 | 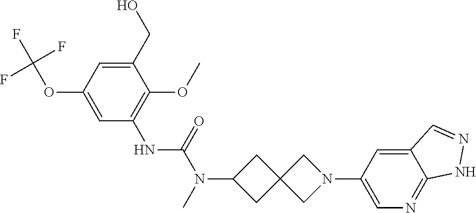 [SMILES: COc1c(CO)cc(OC(F)(F)F)cc1NC(=O)N(C)C1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (3-(hydroxymethyl)-2- methoxy-5- (trifluoromethoxy)phenyl)-1- methylurea |
| 219 | 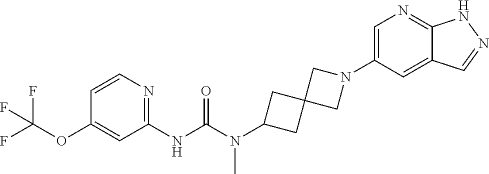 [SMILES: CN(C(=O)Nc1cc(OC(F)(F)F)ccn1)C1CC2(C1)CN(c1cnc3[nH]ncc3c1)C2] | 1-(2-(1H-pyrazolo[3,4- b]pyridin-5-yl)-2- azaspiro[3.3]heptan-6-yl)-1- methyl-3-(4- (trifluoromethoxy)pyridin-2- yl)urea |
| 220 | 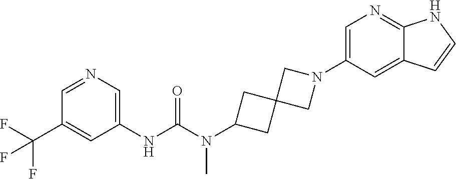 [SMILES: CN(C(=O)Nc1cncc(C(F)(F)F)c1)C1CC2(C1)CN(c1cnc3[nH]ccc3c1)C2] | 1-(2-(1H-pyrrolo[2,3- b]pyridin-5-yl)-2- azaspiro[3.3]heptan-6-yl)-1- methyl-3-(5- (trifluoromethyl)pyridin-3- yl)urea |
| 221 | 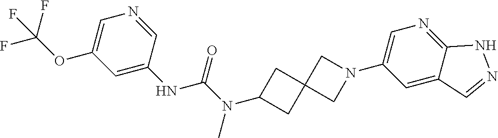 [SMILES: CN(C(=O)Nc1cncc(OC(F)(F)F)c1)C1CC2(C1)CN(c1cnc3[nH]ncc3c1)C2] | 1-(2-(1H-pyrazolo[3,4- b]pyridin-5-yl)-2- azaspiro[3.3]heptan-6-yl)-1- methyl-3-(5- (trifluoromethoxy)pyridin-3- yl)urea |
| 304 | 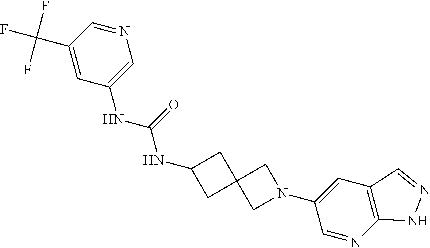 [SMILES: O=C(Nc1cncc(C(F)(F)F)c1)NC1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (5-(trifluoromethyl)pyridin-3- yl)urea |
| 306 | 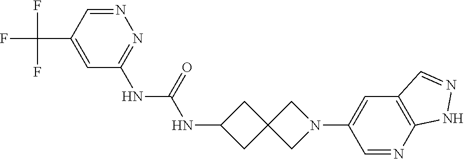 [SMILES: O=C(Nc1cc(C(F)(F)F)cnn1)NC1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (5-(trifluoromethyl)pyridazin- 3-yl)urea |
| 307 | 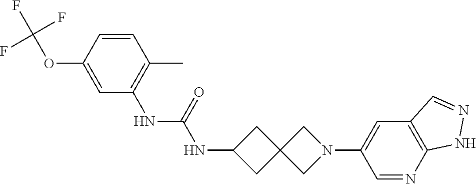 [SMILES: Cc1ccc(OC(F)(F)F)cc1NC(=O)NC1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (2-methyl-5- (trifluoromethoxy)phenyl)urea |
| 312 | 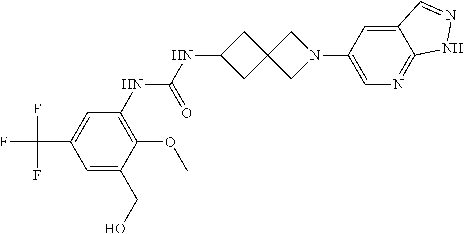 [SMILES: COc1c(CO)cc(C(F)(F)F)cc1NC(=O)NC1CC2(C1)CN(c1cnc3[nH]ncc3c1)C2] | 1-(2-(1H-pyrazolo[3,4- b]pyridin-5-yl)-2- azaspiro[3.3]heptan-6-yl)-3- (3-(hydroxymethyl)-2- methoxy-5- (trifluoromethyl)phenyl)urea |

Additional particularly preferred embodiments of the invention are the compounds of formula (Id) as defined above, wherein A is optionally substituted phenyl or pyridinyl, L is CO and B is selected from pyrazolo[5,1-b]thiazolyl, imidazo[1,2-a]pyridinyl, pyrazolo[1,5-a]pyrazinyl, imidazo[1,5-a]pyrazinyl, thieno[3,2-b]pyridinyl, 1H-imidazo[1,2-b]pyrazolyl and pyrazolo[5,1-b]thiazolyl.

Further particularly preferred compounds of formula (Id) are those listed in Table 4 below, and pharmaceutically acceptable salts thereof. These compounds are particularly active on receptors DDR1 and DDR2, as shown in Table 44 further below.

TABLE 4
List of representative compounds of Formula (Id)

| Ex- | | |
|---|---|---|
| ample | | |
| N^{o} | Structure | Chemical Name |
| 27 | 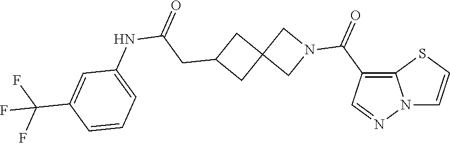 [SMILES: O=C(CC1CC2(C1)CN(C(=O)c1cnn3ccsc13)C2)Nc1cccc(C(F)(F)F)c1] | 2-(2-(pyrazolo[5,1-b]thiazole-7- carbonyl)-2-azaspiro[3.3]heptan- 6-yl)-N-(3- (trifluoromethyl)phenyl)acetamide |
| 28 | 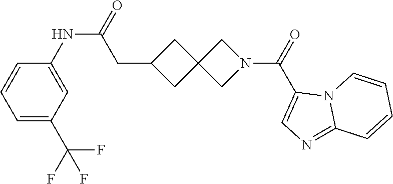 [SMILES: O=C(CC1CC2(C1)CN(C(=O)c1cnc3ccccn13)C2)Nc1cccc(C(F)(F)F)c1] | 2-(2-(imidazo[1,2-a]pyridine-3- carbonyl)-2-azaspiro[3.3]heptan- 6-yl)-N-(3- (trifluoromethyl)phenyl)acetamide |
| 29 | 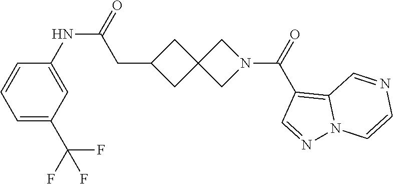 [SMILES: O=C(CC1CC2(C1)CN(C(=O)c1cnn3ccncc13)C2)Nc1cccc(C(F)(F)F)c1] | 2-(2-(pyrazolo[1,5-a]pyrazine-3- carbonyl)-2-azaspiro[3.3]heptan- 6-yl)-N-(3- (trifluoromethyl)phenyl)acetamide |
| 139 | 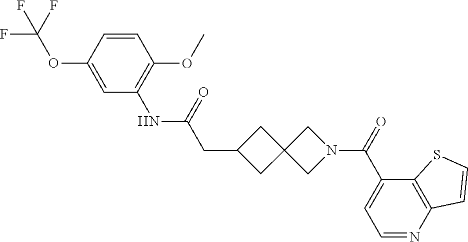 [SMILES: COc1ccc(OC(F)(F)F)cc1NC(=O)CC1CC2(C1)CN(C(=O)c1ccnc3ccsc13)C2] | N-(2-methoxy-5- (trifluoromethoxy)phenyl)-2-(2- (thieno[3,2-b]pyridine-7- carbonyl)-2-azaspiro[3.3]heptan- 6-yl)acetamide |
| 140 | 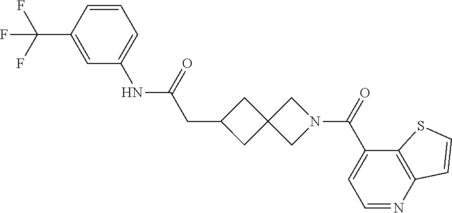 [SMILES: O=C(CC1CC2(C1)CN(C(=O)c1ccnc3ccsc13)C2)Nc1cccc(C(F)(F)F)c1] | 2-(2-(thieno[3,2-b]pyridine-7- carbonyl)-2-azaspiro[3.3]heptan- 6-yl)-N-(3- (trifluoromethyl)phenyl)acetamide |
| 141 | 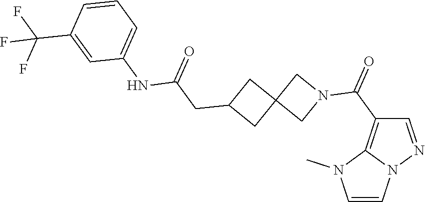 [SMILES: Cn1ccn2ncc(C(=O)N3CC4(CC(CC(=O)Nc5cccc(C(F)(F)F)c5)C4)C3)c12] | 2-(2-(1-methyl-1H-imidazo[1,2- b]pyrazole-7-carbonyl)-2- azaspiro[3.3]heptan-6-yl)-N-(3- (trifluoromethyl)phenyl)acetamide |
| 142 | 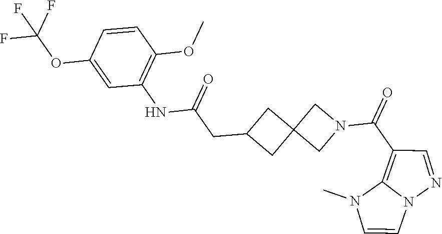 [SMILES: COc1ccc(OC(F)(F)F)cc1NC(=O)CC1CC2(C1)CN(C(=O)c1cnn3ccn(C)c13)C2] | N-(2-methoxy-5- (trifluoromethoxy)phenyl)-2-(2- (1-methyl-1H-imidazo[1,2- b]pyrazole-7-carbonyl)-2- azaspiro[3.3]heptan-6- yl)acetamide |
| 274 | 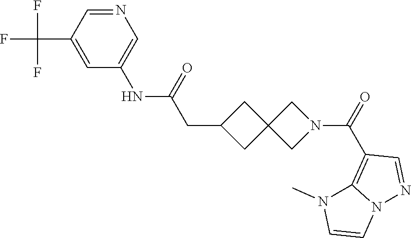 [SMILES: Cn1ccn2ncc(C(=O)N3CC4(CC(CC(=O)Nc5cncc(C(F)(F)F)c5)C4)C3)c12] | 2-(2-(1-methyl-1H-imidazo[1,2- b]pyrazole-7-carbonyl)-2- azaspiro[3.3]heptan-6-yl)-N-(5- (trifluoromethyl)pyridin-3- yl)acetamide |
| 275 | 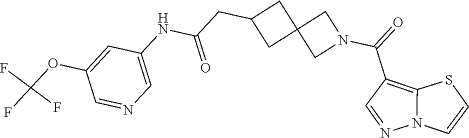 [SMILES: O=C(CC1CC2(C1)CN(C(=O)c1cnn3ccsc13)C2)Nc1cncc(OC(F)(F)F)c1] | 2-(2-(pyrazolo[5,1-b]thiazole-7- carbonyl)-2-azaspiro[3.3]heptan- 6-yl)-N-(5- (trifluoromethoxy)pyridin-3- yl)acetamide |
| 276 | 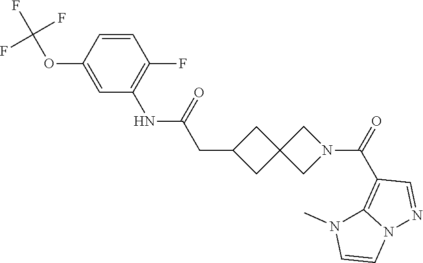 [SMILES: Cn1ccn2ncc(C(=O)N3CC4(CC(CC(=O)Nc5cc(OC(F)(F)F)ccc5F)C4)C3)c12] | N-(2-fluoro-5- (trifluoromethoxy)phenyl)-2-(2- (1-methyl-1H-imidazo[1,2- b]pyrazole-7-carbonyl)-2- azaspiro[3.3]heptan-6- yl)acetamide |
| 277 | 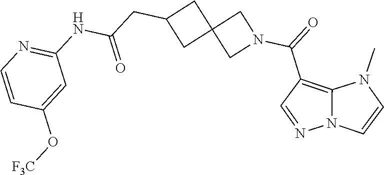 [SMILES: Cn1ccn2ncc(C(=O)N3CC4(CC(CC(=O)Nc5cc(OC(F)(F)F)ccn5)C4)C3)c12] | 2-(2-(1-methyl-1H-imidazo[1,2- b]pyrazole-7-carbonyl)-2- azaspiro[3.3]heptan-6-yl)-N-(4- (trifluoromethoxy)pyridin-2- yl)acetamide |
| 278 | 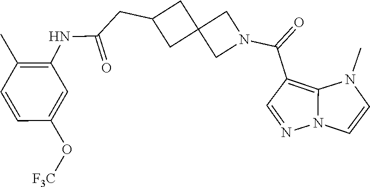 [SMILES: Cc1ccc(OC(F)(F)F)cc1NC(=O)CC1CC2(C1)CN(C(=O)c1cnn3ccn(C)c13)C2] | 2-(2-(1-methyl-1H-imidazo[1,2- b]pyrazole-7-carbonyl)-2- azaspiro[3.3]heptan-6-yl)-N-(2- methyl-5-(trifluoro- methoxy)phenyl)acetamide |
| 279 | 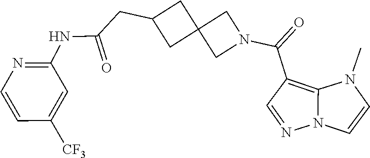 [SMILES: Cn1ccn2ncc(C(=O)N3CC4(CC(CC(=O)Nc5cc(C(F)(F)F)ccn5)C4)C3)c12] | 2-(2-(1-methyl-1H-imidazo[1,2- b]pyrazole-7-carbonyl)-2- azaspiro[3.3]hepan-6-yl)-N-(4- (trifluoromethyl)pyridin-2- yl)acetamide |
| 301 | 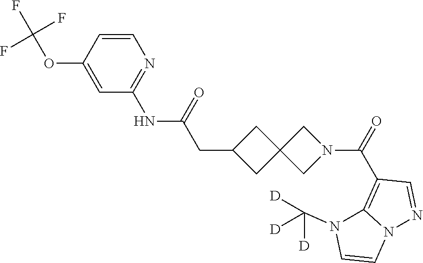 [SMILES: [2H]C([2H])([2H])n1ccn2ncc(C(=O)N3CC4(CC(CC(=O)Nc5cc(OC(F)(F)F)ccn5)C4)C3)c12] | 2-(2-(1-(methyl-d3)-1H- imidazo[1,2-b]pyrazole-7- carbonyl)-2-azaspiro[3.3]heptan- 6-yl)-N-(4- (trifluoromethoxy)pyridin-2- yl)acetamide |
| 302 | 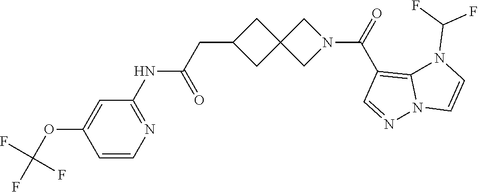 [SMILES: O=C(CC1CC2(C1)CN(C(=O)c1cnn3ccn(C(F)F)c13)C2)Nc1cc(OC(F)(F)F)ccn1] | 2-(2-(1-(difluoromethyl)-1H- imidazo[1,2-b]pyrazole-7- carbonyl)-2-azaspiro[3.3]heptan- 6-yl)-N-(4- (trifluoromethoxy)pyridin-2- yl)acetamide |

Also particularly preferred embodiments of the invention are the compounds of formula (Ie) as defined above, wherein A is selected from the group consisting of optionally substituted phenyl, optionally substituted pyridinyl and optionally substituted isoxazolyl, L is CO and B is selected from pyrazolo[5,1-b]thiazolyl and 1H-imidazo[1,2-b]pyrazolyl.

Further particularly preferred compounds of formula (Ie) are those listed in Table 5 below, and pharmaceutically acceptable salts thereof. These compounds are particularly active on receptors DDR1 and DDR2, as shown in Table 44 further below.

TABLE 5
List of representative compounds of Formula (Ie)

| Ex- | | |
|---|---|---|
| ample | | |
| N^{o} | Structure | Chemical Name |
| 143 | 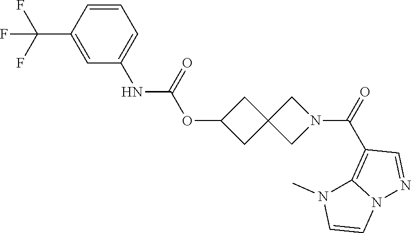 [SMILES: Cn1ccn2ncc(C(=O)N3CC4(CC(OC(=O)Nc5cccc(C(F)(F)F)c5)C4)C3)c12] | 2-(1-methyl-1H-imidazo[1,2- b]pyrazole-7-carbonyl)-2- azaspiro[3.3]heptan-6-yl(3- (trifluoromethyl)phenyl)carbamate |
| 144 | 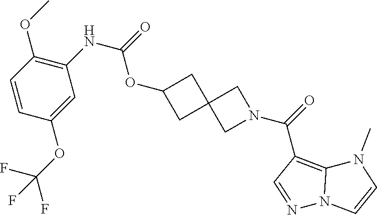 [SMILES: COc1ccc(OC(F)(F)F)cc1NC(=O)OC1CC2(C1)CN(C(=O)c1cnn3ccn(C)c13)C2] | 2-(1-methyl-1H-imidazo[1,2- b]pyrazole-7-carbonyl)-2- azaspiro[3.3]heptan-6-yl(2- methoxy-5- (trifluoromethoxy)phenyl)carba- mate |
| 146 | 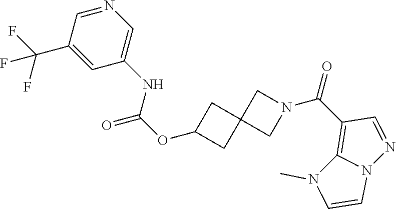 [SMILES: Cn1ccn2ncc(C(=O)N3CC4(CC(OC(=O)Nc5cncc(C(F)(F)F)c5)C4)C3)c12] | 2-(1-methyl-1H-imidazo[1,2- b]pyrazole-7-carbonyl)-2- azaspiro[3.3]heptan-6-yl(5- (trifluoromethyl)pyridin-3- yl)carbamate |
| 147 | 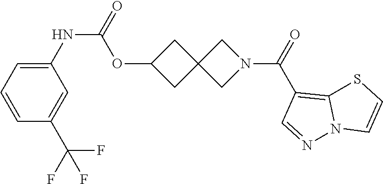 [SMILES: O=C(Nc1cccc(C(F)(F)F)c1)OC1CC2(C1)CN(C(=O)c1cnn3ccsc13)C2] | 2-(pyrazolo[5,1-b]thiazole-7- carbonyl)-2-azaspiro[3.3]heptan- 6-yl(3- (trifluoromethyl)phenyl)carbamate |
| 270 | 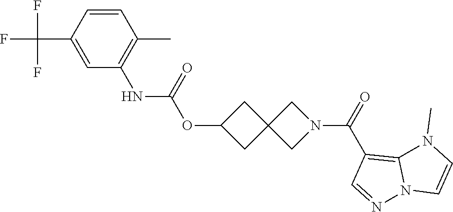 [SMILES: Cc1ccc(C(F)(F)F)cc1NC(=O)OC1CC2(C1)CN(C(=O)c1cnn3ccn(C)c13)C2] | 2-(1-methyl-1H-imidazo[1,2- b]pyrazole-7-carbonyl)-2- azaspiro[3.3]heptan-6-yl(2- methyl-5- (trifluoromethyl)phenyl)carbamate |
| 271 | 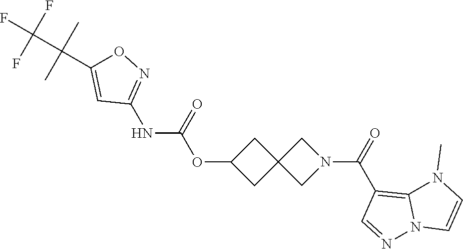 [SMILES: Cn1ccn2ncc(C(=O)N3CC4(CC(OC(=O)Nc5cc(C(C)(C)C(F)(F)F)on5)C4)C3)c12] | 2-(1-methyl-1H-imidazo[1,2- b]pyrazole-7-carbonyl)-2- azaspiro[3.3]heptan-6-yl(5- (1,1,1-trifluoro-2-methylpropan- 2-yl)isoxazol-3-yl)carbamate |
| 272 | 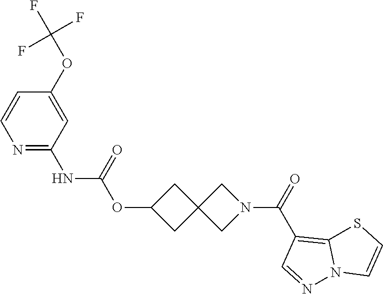 [SMILES: O=C(Nc1cc(OC(F)(F)F)ccn1)OC1CC2(C1)CN(C(=O)c1cnn3ccsc13)C2] | 2-(pyrazolo[5,1-b]thiazole-7- carbonyl)-2-azaspiro[3.3]heptan- 6-yl(4- (trifluoromethoxy)pyridin-2- yl)carbamate |
| 273 | 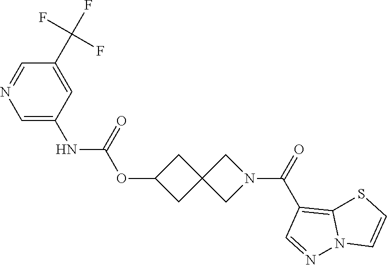 [SMILES: O=C(Nc1cncc(C(F)(F)F)c1)OC1CC2(C1)CN(C(=O)c1cnn3ccsc13)C2] | 2-(pyrazolo[5,1-b]thiazole-7- carbonyl)-2-azaspiro[3.3]heptan- 6-yl(5-(trifluoromethyl)pyridin- 3-yl)carbamate |
| 292 | 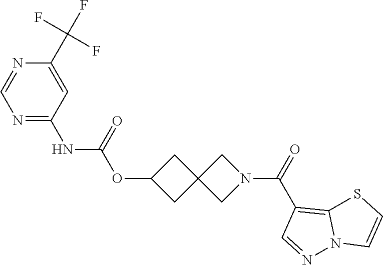 [SMILES: O=C(Nc1cc(C(F)(F)F)ncn1)OC1CC2(C1)CN(C(=O)c1cnn3ccsc13)C2] | 2-(pyrazolo[5,1-b]thiazole-7- carbonyl)-2-azaspiro[3.3]heptan- 6-yl(6- (trifluoromethyl)pyrimidin-4- yl)carbamate |
| 293 | 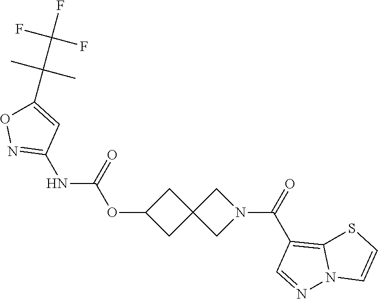 [SMILES: CC(C)(c1cc(NC(=O)OC2CC3(C2)CN(C(=O)c2cnn4ccsc24)C3)no1)C(F)(F)F] | 2-(pyrazolo[5,1-b]thiazole-7- carbonyl)-2-azaspiro[3.3]heptan- 6-yl(5-(1,1,1-trifluoro-2- methylpropan-2-yl)isoxazol-3- yl)carbamate |
| 294 | 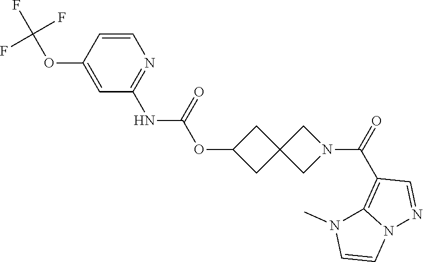 [SMILES: Cn1ccn2ncc(C(=O)N3CC4(CC(OC(=O)Nc5cc(OC(F)(F)F)ccn5)C4)C3)c12] | 2-(1-methyl-1H-imidazo[1,2- b]pyrazole-7-carbonyl)-2- azaspiro[3.3]heptan-6-yl(4- (trifluoromethoxy)pyridin-2- yl)carbamate |
| 295 | 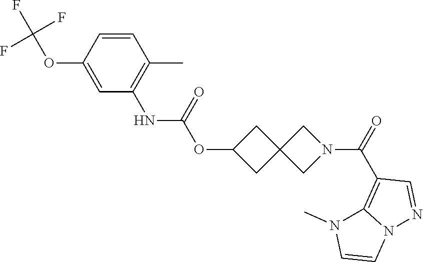 [SMILES: Cc1ccc(OC(F)(F)F)cc1NC(=O)OC1CC2(C1)CN(C(=O)c1cnn3ccn(C)c13)C2] | 2-(1-methyl-1H-imidazo[1,2- b]pyrazole-7-carbonyl)-2- azaspiro[3.3]heptan-6-yl(2- methyl-5- (trifluoromethoxy)phenyl)carba- mate |
| 296 | 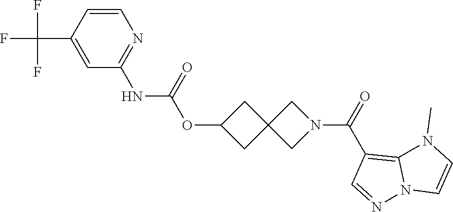 [SMILES: Cn1ccn2ncc(C(=O)N3CC4(CC(OC(=O)Nc5cc(C(F)(F)F)ccn5)C4)C3)c12] | 2-(1-methyl-1H-imidazo[1,2- b]pyrazole-7-carbonyl)-2- azaspiro[3.3]heptan-6-yl(4- (trifluoromethyl)pyridin-2- yl)carbamate |
| 297 | 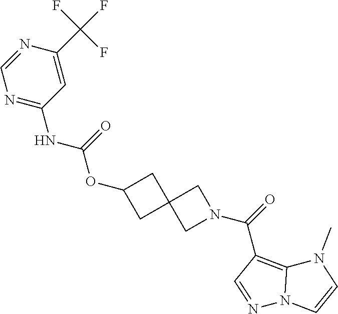 [SMILES: Cn1ccn2ncc(C(=O)N3CC4(CC(OC(=O)Nc5cc(C(F)(F)F)ncn5)C4)C3)c12] | 2-(1-methyl-1H-imidazo[1,2- b]pyrazole-7-carbonyl)-2- azaspiro[3.3]heptan-6-yl(6- (trifluoromethyl)pyrimidin-4- yl)carbamate |
| 299 | 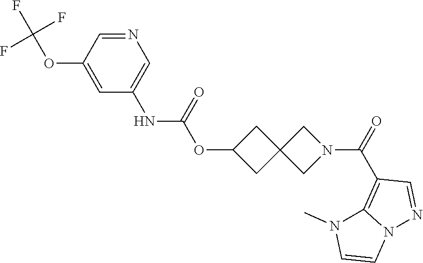 [SMILES: Cn1ccn2ncc(C(=O)N3CC4(CC(OC(=O)Nc5cncc(OC(F)(F)F)c5)C4)C3)c12] | 2-(1-methyl-1H-imidazo[1,2- b]pyrazole-7-carbonyl)-2- azaspiro[3.3]heptan-6-yl(5- (trifluoromethoxy)pyridin-3- yl)carbamate |
| 311 | 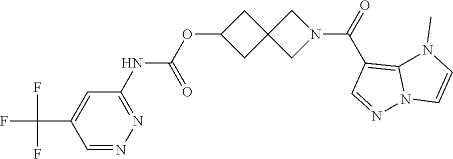 [SMILES: Cn1ccn2ncc(C(=O)N3CC4(CC(OC(=O)Nc5cc(C(F)(F)F)cnn5)C4)C3)c12] | 2-(1-methyl-1H-imidazo[1,2- b]pyrazole-7-carbonyl)-2- azaspiro[3.3]heptan-6-yl(5- (trifluoromethyl)pyridazin-3- yl)carbamate |

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimal reaction conditions may vary depending on the particular reactants or solvent used, such conditions can be readily determined by those skilled in the art by routine optimization procedures. Thus, processes described below should not be viewed as limiting the scope of the synthetic methods available for the preparation of the compounds of the invention.

In some cases, generally known protective groups (PG) may be employed when needed to mask or protect sensitive or reactive moieties, in accordance to general principles of chemistry (Protective group in organic syntheses, 3^{rd} ed. T. W. Greene, P. G. M. Wuts).

Compounds of formula (I) may be prepared as described hereinafter in SCHEME 1 to SCHEME 6, wherein at least one non-limiting synthetic route is provided for the preparation of the exemplified compounds (i.e. the Examples).

Compounds of formula (I), in particular compounds of formula (Ia), wherein L is CO and L1 is NR, may be prepared, for instance, according to SCHEME 1 as described below.

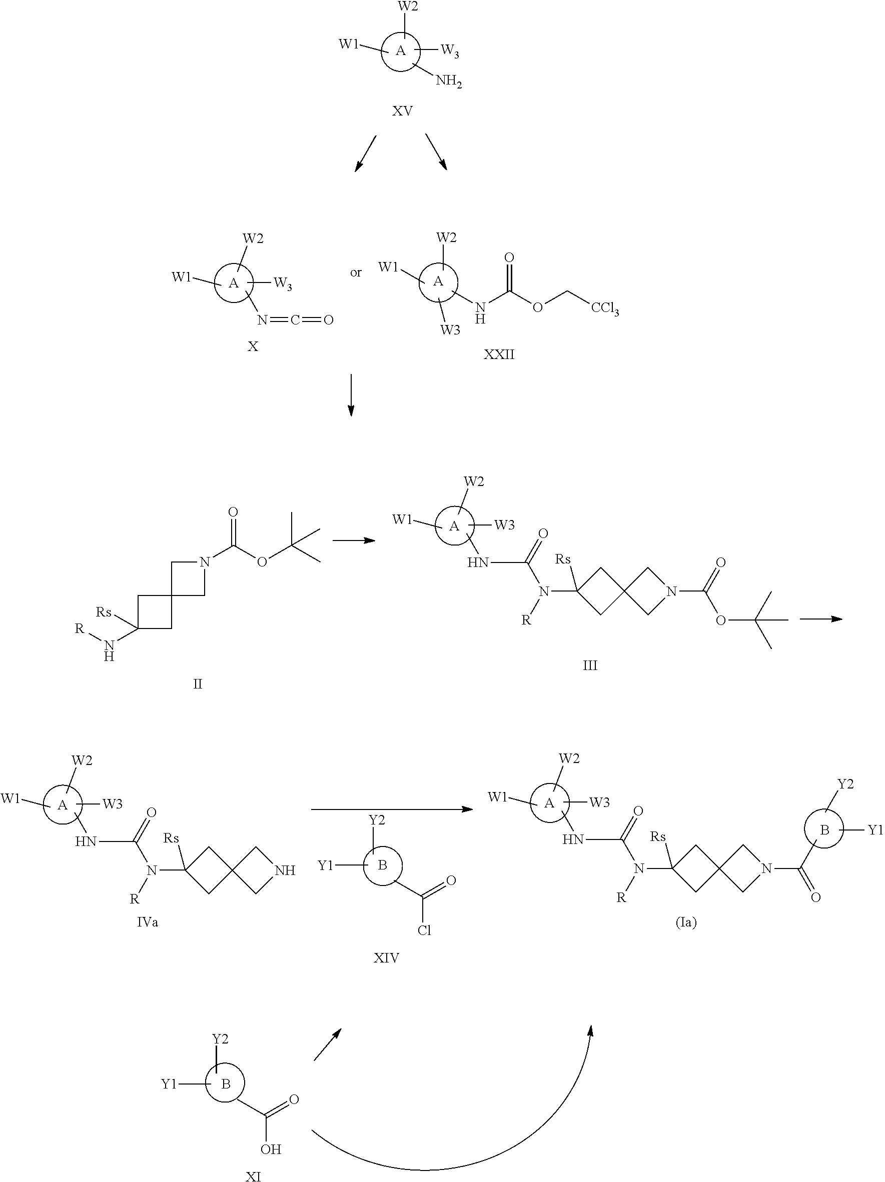

According to SCHEME 1, intermediate urea III may be prepared from amine II, wherein Rs is hydrogen or methyl, by reaction with the isocyanate X in a suitable solvent, such as DCM, at rt. Intermediate compound IVa may then be obtained from intermediate III by Boc-deprotection in acidic conditions, for instance by using TFA in a suitable solvent, such as DCM, at rt.

Alternatively, intermediate urea III may be prepared from amine II by reaction with the suitable 2,2,2-trichloroethyl carbamate (Troc carbamate) XXII, which can be obtained from the suitable amine XV and 2,2,2-trichloroethyl chloroformate, in a suitable solvent mixture, such as ACN, in the presence of a base, such as DIPEA, and at a temperature varying from 80° C. to 150° C. in a microwave oven.

Subsequently, carboxylic acid XI and intermediate compound IVa may be reacted to give a compound of formula (Ia) under suitable amide coupling reaction conditions, in the presence of an activating agent, such as for instance HATU or TBTU, with an organic base, such as DIPEA or TEA, in a suitable organic solvent, such as DCM or DMF, at a temperature generally around rt for a time ranging from a few hours to overnight. Alternatively, a compound of formula (Ia) may be prepared from amine IVa and acyl chloride XIV, which can be obtained from carboxylic acid XI in the presence of an appropriate chlorinating reagent, such as POCl₃, thionyl chloride or oxalyl chloride, in a solvent, such as DCM and, if needed, in the presence of a catalytic amount of DMF, at a temperature ranging from 5° C. to 50° C. Alternatively, a compound of formula (Ia) may be prepared via direct amidation of carboxylic acid XI, by conversion into the transient activated acylimidazolinium intermediate in the presence of TCFH and 1-methylimidazole and subsequent reaction with the amine IVa in a solvent, such as DMF, generally around RT.

A compound of formula (Ia), wherein Y1 is bromine, may be converted into a different compound of formula (Ia), wherein Y1 is optionally substituted phenyl or optionally substituted heteroaryl, by reaction with a phenyl or heteroaryl boronic ester/acid by a palladium-catalyzed cross-coupling. The reaction may be carried out by the usual Suzuki protocol, in a suitable organic solvent, such as dioxane, in the presence of an inorganic base, such as potassium carbonate, with an appropriate palladium catalytic system, such as Pd(dppf)Cl₂, at high temperature (around 100° C.) for few hours. A compound of formula (Ia), wherein Y1 is bromine, may be converted into a different compound of formula (Ia), wherein Y1 is heterocycloalkyl, by reaction with an heterocycloalkylamine (i.e. a heterocycloalkyl wherein a group NH is part of the ring) by means of an electrochemical reaction, in the presence of dibromonickel and an electrolyte, such as TBAB, in an appropriate solvent, such as DMA.

Compounds of formula (I), in particular compounds of formula (Ib), wherein L is CH₂ and L1 is NR, or compounds of formula (Ic), wherein L is absent and L1 is NR, may be prepared, for instance, according to SCHEME 2 as described below.

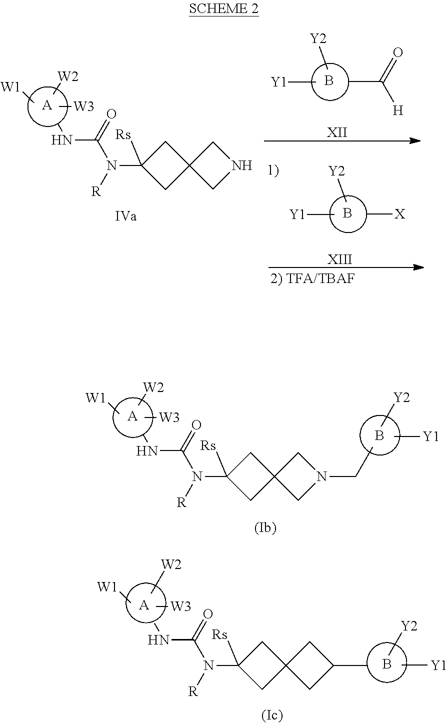

As depicted in SCHEME 2, intermediate compound IVa may be converted into a compound of formula (Ib) by applying reductive amination conditions with the appropriate aldehyde XII, with a suitable reducing agent, such as STAB or NaCNBH₃, in a suitable solvent, such as DCM or EtOH, in the presence of an acid, such as acetic acid, and in the presence of a dehydrating agent, such as magnesium sulfate, if needed, at a temperature ranging from rt to 50° C.

Otherwise, intermediate compound IVa may be converted into a compound of formula (Ic) by cross-coupling reactions, like for instance Buchwald coupling, in the presence of a palladium catalyst, such as Pd(dppf)Cl₂, or another palladium source/phosphine-based ligand at high temperature (around 100° C.) for a few hours with a suitable aryl halide XIII, preferably an aryl bromide (X=Br), in an organic solvent, such as DMA, with a suitable base, such as Cs₂CO₃.

In case a protecting group is present on aryl halide XIII or intermediate compound IVa, a compound of formula (I) may be obtained after an additional step of removal of the protecting group in the required conditions, such as acidic conditions, by using TFA or HCl in 4-dioxane, or with TBAF.

Compounds of formula (Ic) may also be prepared according to SCHEME 2a as described hereinafter.

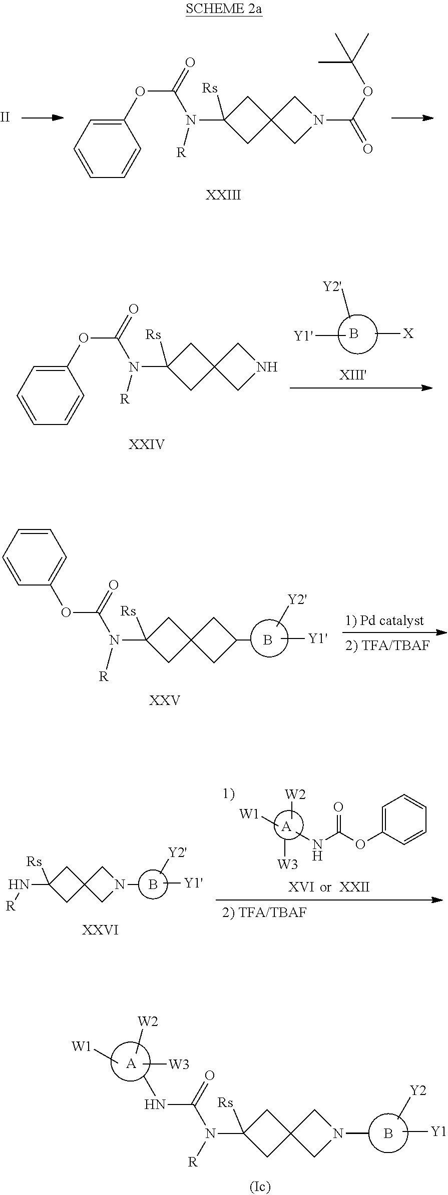

According to SCHEME 2a, intermediate compound XXIII can be obtained by protection of amine II, as defined above, with phenyl chloroformate, in a suitable solvent, such as for instance DCM or EtOAc and/or water, at a temperature varying from −5° C. to rt, optionally in the presence of TEA. Similarly, N-(benzyloxycarbonyloxy) succinimide or 4-nitrophenyl chloroformate may be used for protection of amine II. Intermediate compound XXIV may then be prepared from intermediate compound XXIII by Boc-deprotection in acidic conditions, for instance by using TFA in a suitable solvent, such as DCM, at rt. Intermediate compound XXIV may be converted into intermediate compound XXV by cross-coupling reactions, like for instance Buchwald coupling, with a suitable aryl halide XIII′, in the presence of a palladium catalyst, such as Pd-175 or Pd(dppf)Cl₂, or another palladium source/phosphine-based ligand, at high temperature (around 100° C.) for a few hours, in an organic solvent, such as DMA or Me-THF, with a suitable base, such as Cs₂CO₃, and wherein Y1′/Y2′ may be Y1/Y2 or may bear protecting groups, such as Boc or SEM, and wherein the halide X is preferably bromide. Then compounds of formula XXVI may be prepared by amine deprotection reaction of the intermediate compound XXV in the presence of a palladium catalyst and triethylsilane or Pd/C 10% and hydrogen, in a suitable solvent, such as ethanol, at a temperature generally around rt for a few hours, optionally followed by deprotection of any protecting group on the Y substituents, if present, in acidic conditions, for instance by using TFA in a suitable solvent, such as DCM, or using TBAF in THE, at rt.

Then, compounds of formula (Ic) may be prepared by reaction of the intermediate compound XXVI either with the suitable 2,2,2-trichloroethyl carbamate (Troc carbamate) XXII, which can be obtained from the suitable amine XV and 2,2,2-trichloroethyl chloroformate, or with intermediate compound XVI, which can be obtained from the suitable amine XV and phenyl chloroformate, in a suitable solvent mixture, such as ACN or THF, in the presence of a base, such as DIPEA, and at a temperature varying from rt to 150° C., in a few cases in a microwave oven, followed by deprotection of any protecting group on the W1, W2 or W3 substituents. For instance, acidic conditions may be used for cleavage of SEM protecting group, such as TFA in DCM or TBAF in THF, at rt.

Compounds of formula (Ia) may also be prepared according to SCHEME 3 as described hereinafter.

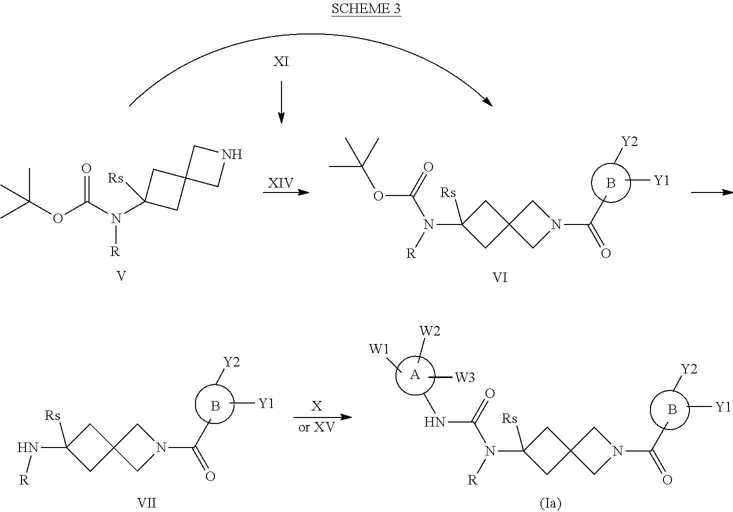

Intermediate VI may be prepared from amine V and acyl chloride XIV (as defined in SCHEME 1), which can be obtained from carboxylic acid XI (as defined in SCHEME 1) in the presence of an appropriate chlorinating reagent, such as POCl₃, thionyl chloride or oxalyl chloride, in a solvent, such as DCM and, if needed, in the presence of a catalytic amount of DMF at a temperature ranging from 5° C. to 50° C.; the amine V and the acyl chloride XIV may undergo an amide coupling in the presence of a suitable base, such as TEA, in a suitable solvent, such as DCM, at rt. Alternatively, intermediate VI may be prepared via direct amidation of carboxylic acid XI, by conversion into the transient activated acylimidazolinium intermediate in the presence of TCFH and 1-methylimidazole and subsequent reaction with the amine V in a solvent, such as DMF, generally at rt. Intermediate VII may then be obtained from intermediate VI by Boc-deprotection in acidic conditions, for instance by using TFA in a suitable solvent, such as DCM, at rt. Compounds of formula (Ia) may be prepared from intermediate VII by reaction with the isocyanate X (see SCHEME 1) in a suitable solvent, such as DCM, at rt. Alternatively, compounds of formula (Ia) may be prepared from intermediate VII by reaction with amine XV (see SCHEME 1) and triphosgene in a suitable solvent, such as DCM, at a temperature varying from 0° C. to rt.

Additionally, compounds of formula (Ic) may be prepared according to SCHEME 3a as described hereinafter.

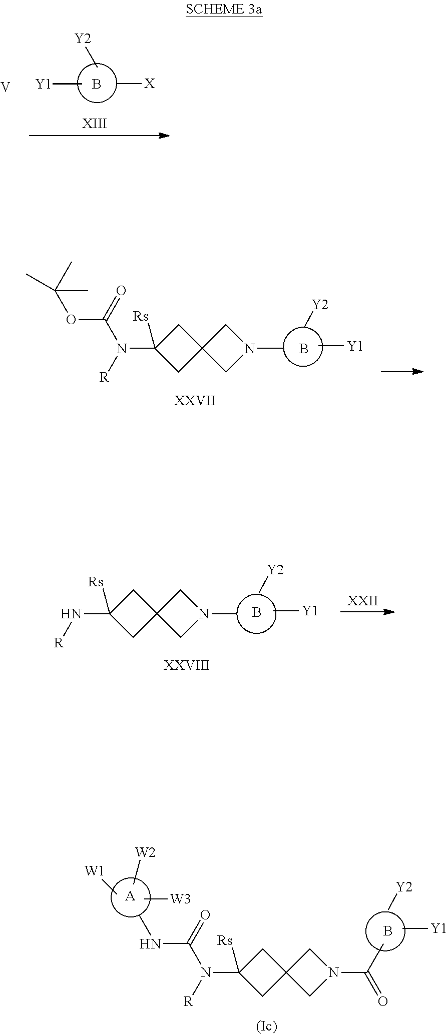

Intermediate compound XXVII may be obtained by cross-coupling reactions, like for instance Buchwald coupling, starting from amine V in the presence of a palladium catalyst, such as Pd(dppf)Cl₂, or another palladium source/phosphine-based ligand at high temperature (around 100° C.) for a few hours with a suitable aryl halide XIII, preferably an aryl bromide (X=Br), in an organic solvent, such as DMA, with a suitable base, such as Cs₂CO₃. Intermediate compound XXVIII may then be obtained from intermediate XXVII by Boc-deprotection in acidic conditions, for instance by using TFA in a suitable solvent, such as DCM, at rt. Compounds of formula (Ic) may be prepared from intermediate XXVIII by reaction with the suitable 2,2,2-trichloroethyl carbamate (Troc carbamate) XXII, which can be obtained from the suitable amine XV (see SCHEME 1) and 2,2,2-trichloroethyl chloroformate, in a suitable solvent mixture, such as ACN, in the presence of a base, such as DIPEA, and at a temperature varying from 80° C. to 150° C. in a microwave oven.

Compounds of formula (Ia) may also be prepared according to SCHEME 4 as described hereinafter.

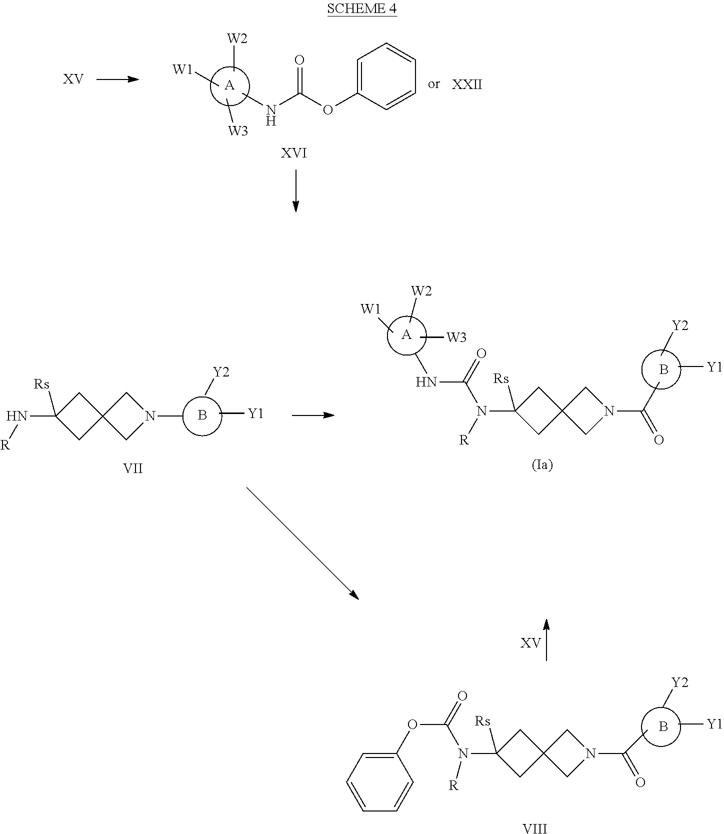

Compounds of formula (Ia) may be prepared from intermediate VII by reaction with the suitable phenyl carbamate XVI, which can be obtained from the suitable amine XV (see SCHEME 1) and phenyl chloroformate in a suitable solvent mixture, such as a mixture of EtOAc, DCM and water, at a temperature varying from 0° C. to rt. Alternatively, the suitable amine XV may be reacted with N-(benzyloxycarbonyloxy) succinimide, in a suitable solvent, such as DCM, at a temperature varying from −5° C. to rt, in the presence of TEA to afford the benzyloxycarbonyl protected amine XV.

Compounds of formula (Ia) may also be prepared from intermediate VII by reaction with the suitable 2,2,2-trichloroethyl carbamate (Troc carbamate) XXII (see SCHEME 1), which can be obtained as described above.

Alternatively, compounds of formula (Ia) may be prepared by reaction of the suitable amine XV with intermediate VIII, which may be obtained from intermediate VII and phenyl chloroformate in a suitable solvent mixture, such as a mixture of EtOAc, DCM and water, at a temperature varying from 0° C. to rt.

Compounds of formula (Id), which are compounds of formula (I) wherein L1 is CH₂, may be prepared, for instance, according to SCHEME 5 as described below.

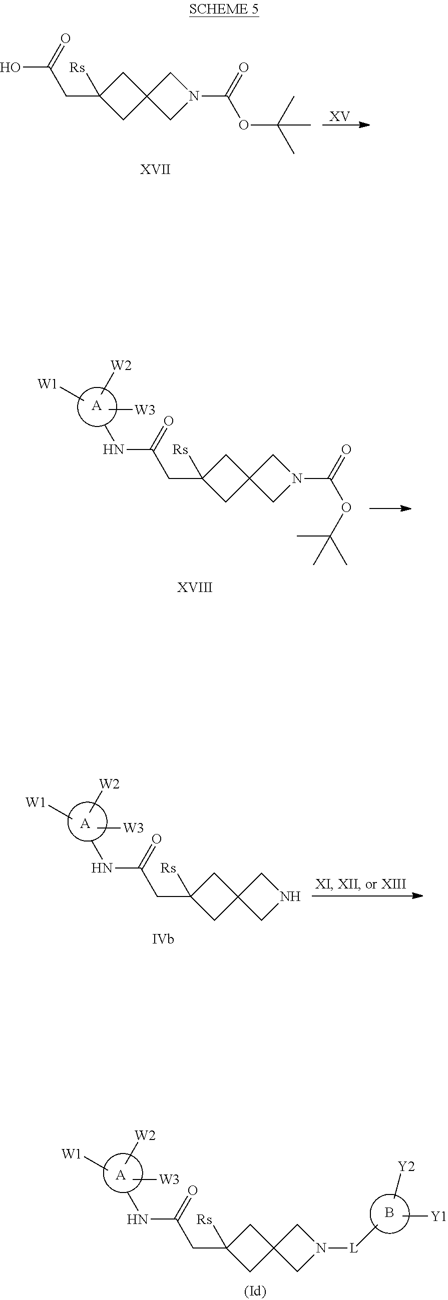

According to SCHEME 5, intermediate XVIII may be prepared from commercially available carboxylic acid XVII, wherein Rs is hydrogen, by reaction with the suitable amine XV (see SCHEME 1) under suitable amide coupling reaction conditions, in the presence of an activating agent, such as for instance HATU or TBTU, and an organic base, such as DIPEA or TEA, in a suitable organic solvent, such as DCM or DMF, and at a temperature generally around rt for a time ranging from a few hours to overnight. Alternatively, a compound XVIII may be prepared via direct amidation of carboxylic acid XVII, i.e. by its conversion into the transient activated acylimidazolinium intermediate in the presence of TCFH and 1-methylimidazole and subsequent reaction with the amine XV in a solvent, such as DMF, generally around rt.

Intermediate compound IVb may then be obtained from intermediate XVIII by Boc-deprotection in acidic conditions, for instance by using TFA in a suitable solvent, such as DCM, at rt. Subsequently, intermediate compound IVb may be reacted with either a carboxylic acid XI, as described for SCHEME 1, or an aldehyde XII or an aryl halide XIII, as described for SCHEME 2, to obtain a compound of formula (Id), wherein L is CO, or CH₂, or absent, respectively. Intermediate compound IVb may also be reacted with an aryl halide XIII′, followed by a deprotection reaction, as described for the synthetic pathway of SCHEME 2a, wherein XIII′ is defined, to finally provide a compound of formula (Id).

Compounds of formula (Ie), which are compounds of formula (I) wherein L1 is O, may be prepared, for instance, according to SCHEME 6 as described below.

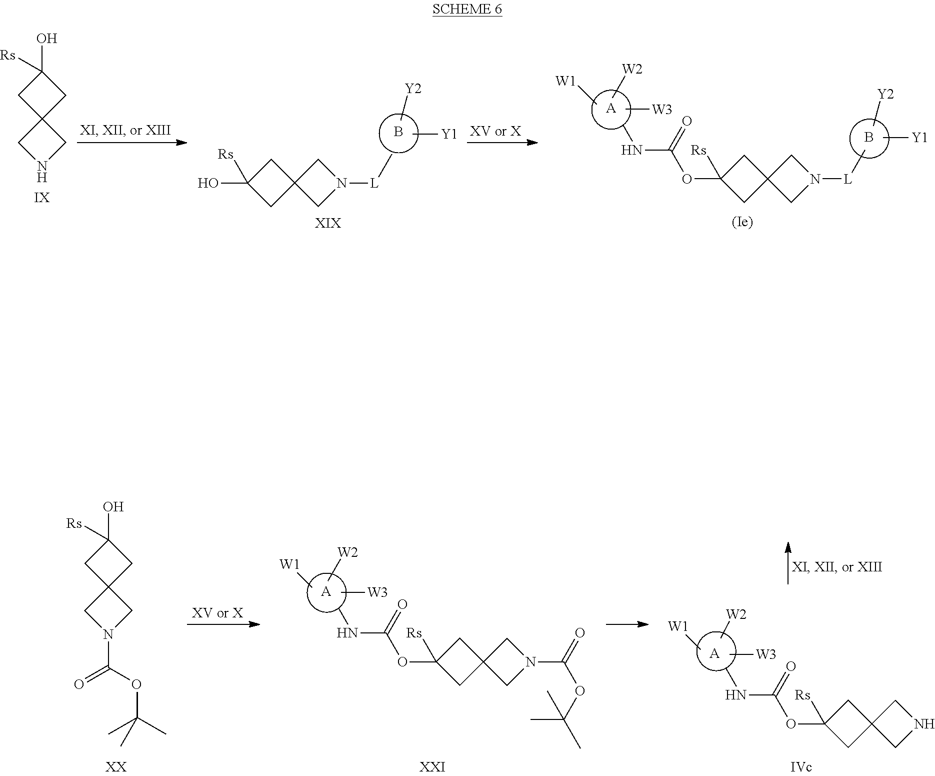

According to SCHEME 6, commercially available compound IX, wherein Rs is hydrogen, may be reacted with either a carboxylic acid XI (see SCHEME 1) or an aldehyde XII or an aryl halide XIII (see SCHEME 2) to obtain intermediate compound XIX, wherein L is CO, or CH₂, or absent, respectively. Compounds of formula (Ie) may be prepared from intermediate compound XIX by reaction with a chloroformate, such as 4-nitrophenyl chloroformate, and the suitable amine XV (see SCHEME 1) in the presence of a base, such as pyridine, in a suitable solvent, such as DCM, at rt. Alternatively, compounds of formula (Ie) may be prepared from intermediate compound XIX by reaction with isocyanate X (see SCHEME 1) in a suitable solvent, such as DCM, and in the presence of a base, such as DIPEA, at rt.

According to another synthetic approach, commercially available compound XX may be reacted with isocyanate X in a suitable solvent, such as DCM, and in the presence of a base, such as DIPEA, at rt, or compound XX may be reacted with a suitable chloroformate and amine XV, in the presence of a base, such as pyridine, in a suitable solvent, such as DCM, at rt, thus affording intermediate compound XXI. Intermediate IVc may then be obtained from intermediate XXI by Boc-deprotection in acidic conditions, for instance by using TFA in a suitable solvent, such as DCM, at rt. Then intermediate IVc may be reacted with either a carboxylic acid XI or an aldehyde XII or an aryl halide XIII, as described for SCHEME 1 and 2, to obtain a compound of formula (Ie), wherein L is CO, or CH₂, or absent, respectively. Intermediate compound IVc may also be reacted with an aryl halide XIII′, followed by a deprotection reaction, as described for the synthetic pathway of SCHEME 2a, wherein XIII′ is defined, to finally provide a compound of formula (Ie).

Accordingly, the present invention provides an intermediate compound IV

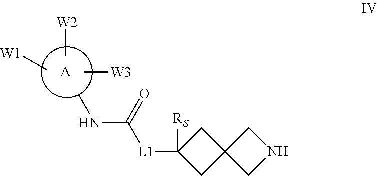

wherein
A is a ring selected from the group consisting of:

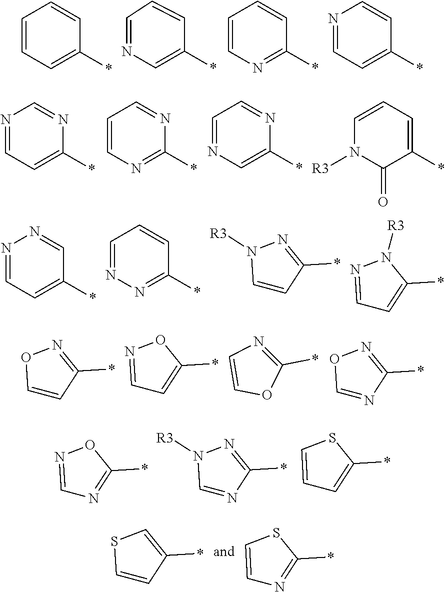

wherein [symbol] indicates a direct bond to NH;
   W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇) cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇) cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl, and are preferably selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy, CH₂N (CH₃)₂ and 4-methylpiperazin-1-yl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms; and wherein preferably the 6-membered heterocycloalkyl is a pyranyl ring;
   L1 is selected from NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇) cycloalkyl-(C₁-C₄)alkyl and deuterated (C₁-C₄)alkyl, preferably selected from the group consisting of hydrogen, CD₃, cyclopropylmethyl, cyclopropyl, CH₂CF₃, CH₂CH₃ or CH₃;
   Rs is selected from hydrogen and methyl when L1 is NR;
   Rs is hydrogen when L1 is CH₂ or O;
   R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl; preferably, R1 and R2 are independently selected from the group consisting of hydrogen, (C₁-C₄)alkyl and optionally substituted heterocycloalkyl, wherein optional substituents, when present, are selected from the group consisting of CH₃, OCH₃, OCF₃, CF₃, C(CH₃)₃, C(CH₃)₂CF₃, CONH₂, OCF₂H and CF₂H;
   more preferably, R1 and R2 are independently selected from the group consisting of hydrogen, methyl and optionally substituted oxetanyl; and
   R3 is selected from the group consisting of (C₁-C₄)alkyl, (C₁-C₄)haloalkyl, (C₁-C₄)alkyl-phenyl and monocyclic heteroaryl;
   or a stereoisomer, tautomer, solvate and pharmaceutically acceptable salt thereof;
   wherein the intermediate compound IV is not 1-(3,5-dichlorophenyl)-3-(2-azaspiro[3.3]heptan-6-yl)urea;
   and wherein, when A ring is phenyl, L1 is NH and W1 and W2 are hydrogen, W3 is not para-OCF₃.

The intermediate compound IV is not 1-(2-azaspiro[3.3]heptan-6-yl)-3-(4-(trifluoromethoxy)phenyl)urea.

The intermediate compound IV corresponds to an intermediate compound IVa when L1 is NR, to an intermediate compound IVb when L1 is CH₂ and to an intermediate compound IVc when L1 is O.

Preferably, substituents W1, W2 and W3 (indicated also as W substituents) in the intermediate compound IV occupy ortho and/or meta positions on ring A. More preferred W substituents are hydrogen, CH₃, OCH₃, OCF₃, CF₃, CHF₂, CH₂CF₃, OCF₂H, CH₂OH, SF₅, F, Br and I. More preferably, such substituents occupy ortho and/or meta positions. When W1 and W2 are both hydrogen and W3 is OCF₃, W3 preferably occupies ortho or meta position.

When A ring is phenyl, L1 is NH and W1 and W2 are hydrogen, W3 is not para-OCF₃.

When A ring is phenyl, L1 is NH and W1 is hydrogen, W2 and W3 are not both Cl in meta positions.

In one preferred embodiment, when A ring is phenyl and L1 is NH, W1, W2 and W3 are independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇) cycloalkyl, heterocycloalkyl-(C₁-C₄)alkyl, heterocycloalkyl-NH—(C₁-C₄)alkyl, (C₃-C₇) cycloalkyl-(C₁-C₄)alkyl, heterocycloalkyl-(C₁-C₄)alkoxy, (C₁-C₄)alkyl-CO-heterocycloalkyl-oxy, (C₃-C₇) cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms.

The invention further provides the use of the intermediate compound IV as defined above in the preparation of a compound of formula (I), or pharmaceutically acceptable salts thereof.

Accordingly, the present invention provides a process for the preparation of a compound of formula (I), or a pharmaceutically acceptable salt thereof, comprising the step of:
a) reacting an intermediate compound IV

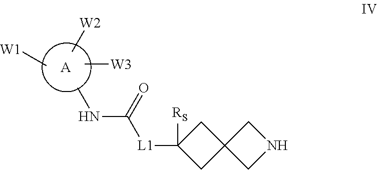

with either a carboxylic acid XI or an aldehyde XII or an aryl halide XIII, as defined above, to obtain a compound of formula (I), as defined above, wherein A, L1, Rs, W1, W2 and W3 are as defined above and wherein X in the aryl halide XIII is a halogen, preferably bromine.

The process may comprise an additional deprotection step in case any substituent on A or B rings bears a protecting group, which the skilled in the art would envisage to use to accomplish the synthesis of a compound of formula (I).

Accordingly, the present invention provides intermediate compounds III, IV, IVa, IVb, IVc, VI, VII, VIII, XVIII, XIX, XXI, XXIII, XXIV, XXV, XXVI, XXVII and XXVIII, as defined above, and their use in the preparation of compounds of formula (I).

The compounds of formula (I) of the present invention have surprisingly been found to effectively inhibit both receptor DDR1 and DDR2. Advantageously, the inhibition of receptors DDR1 and DDR2 may result in efficacious treatment of the diseases or conditions wherein the DDR receptors are involved.

In this respect, it has been found that the compounds of formula (I) of the present invention have a very high antagonist drug potency on DDR1 and DDR2. Table 44 in the present experimental part reports such potency expressed as inhibition constant Ki for representative compounds of formula (I) of the invention. Preferred compounds of the invention have a Ki on DDR1 and DDR2 which is lower than 100 nM. More preferred compounds have a Ki between 25 and 5 nM. Even more preferred compounds of the invention have a Ki on DDR1 and DDR2 lower than 5 nM.

In one aspect, the present invention refers to a compound of formula (I) according to any of the embodiments disclosed above for use as a medicament.

In a preferred aspect, the invention refers to a compound of formula (I), and pharmaceutically acceptable salts thereof, for use in treating diseases, disorders, or conditions associated with dysregulation of DDR.

In another aspect, the invention refers to the use of a compound of formula (I) as above described, and pharmaceutically acceptable salts thereof, in the preparation of a medicament for the treatment of disorders associated with dysregulation of DDR.

In another preferred aspect, the invention refers to a compound of formula (I), and pharmaceutically acceptable salts thereof, for use in the prevention and/or treatment of a disease, disorder or condition associated with DDR receptor mechanism. In a preferred embodiment, the present invention refers to a compound of formula (I) for use in the prevention and/or treatment of fibrosis and/or diseases, disorders or conditions that involve fibrosis.

The terms “fibrosis” or “fibrosing disorder,” as used herein, refer to conditions that are associated with the abnormal accumulation of cells and/or fibronectin and/or collagen and/or increased fibroblast recruitment and include, but are not limited to, fibrosis of individual organs or tissues such as the heart, kidney, liver, joints, lung, pleural tissue, peritoneal tissue, skin, cornea, retina, musculoskeletal and digestive tract.

Preferably, the compounds of formula (I) as above described are useful for the treatment and/or prevention of fibrosis, such as pulmonary fibrosis, idiopathic pulmonary fibrosis (IPF), hepatic fibrosis, progressive pulmonary fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis. More preferably, the compounds of formula (I) as above described are useful for the treatment of idiopathic pulmonary fibrosis (IPF).

In another preferred embodiment, the compounds of formula (I) as above described are useful for the treatment of kidney fibrosis.

Thus in a preferred embodiment, the invention refers to a compound of formula (I) or its pharmaceutical composition for use in the prevention and/or treatment of IPF.

In another preferred embodiment, the invention refers to the use of the compounds of formula (I) or its pharmaceutical composition for the preparation of a medicament for the treatment and/or prevention of IPF.

In another preferred embodiment, the invention refers to a method for the treatment and/or prevention of IPF, the method comprising administering a compound of formula (I) or a pharmaceutical composition comprising a compound of formula (I) and one or more pharmaceutically acceptable carriers and/or excipients.

In another preferred embodiment, the invention refers to a compound of formula (I) or its pharmaceutical composition for use in the prevention and/or treatment of kidney fibrosis.

In another aspect, the invention refers to the use of the compounds of formula (I) or its pharmaceutical composition for the preparation of a medicament for the treatment and/or prevention of kidney fibrosis.

In another preferred embodiment, the invention refers to a method for the treatment and/or prevention of kidney fibrosis, the method comprising administering a compound of formula (I) or a pharmaceutical composition comprising a compound of formula (I) and one or more pharmaceutically acceptable carriers and/or excipients.

In one aspect, the invention also refers to a method for the prevention and/or treatment of diseases, disorders or conditions associated with DDR receptors mechanisms, said method comprising the step of administering to a patient in need thereof a therapeutically effective amount of a compound of formula (I) as above described.

In a further aspect, the invention refers to the use of a compound of formula (I) as above described for the treatment of diseases, disorders or conditions associated with DDR receptors mechanism.

In another aspect, the invention refers to the use of a compound of formula (I) as above described in the preparation of a medicament for the treatment of diseases, disorders or conditions associated with DDR receptors mechanism.

In a further aspect, the invention refers to a method for the prevention and/or treatment of a disease, disorder or condition associated with the dysregulation of DDR receptors 1 and 2, said method comprising administering to a patient in need of such treatment a therapeutically effective amount of a compound of formula (I) as above described.

In a further aspect, the present invention refers to the use of a compound of formula (I) as above described for the treatment of a disease, disorder or condition associated with dysregulation of DDR receptors 1 and 2.

As used herein, “safe and effective amount” in reference to a compound of formula (I) or a pharmaceutically acceptable salt thereof or other pharmaceutically active agent means an amount of the compound sufficient to treat the patient's condition but low enough to avoid serious side effects and that can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) according to any of its embodiment in admixture with at least one or more pharmaceutically acceptable carrier and/or excipient.

In one embodiment, the invention refers to a pharmaceutical composition of compounds of formula (I) in admixture with at least one or more pharmaceutically acceptable carrier and/or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage form such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula (I) may be combined with each other and apply as well mutatis mutandis.

The various aspects of the invention described in this application are illustrated by the following examples which are not meant to limit the invention in any way.

### PREPARATION OF INTERMEDIATES AND EXAMPLES

Chemical Names of the compounds were generated with Structure-To-Name tool of PerkinElmer ChemDraw® Professional application (v. 20.0.0.41.) or are common chemical names, unless otherwise stated. All reagents, for which the synthesis is not described in the experimental part, are either commercially available, or are known compounds or may be formed from known compounds by known methods by a person skilled in the art.

In the procedures that follow, some of the starting materials are identified through an “Intermediate” or “Example” number with indications on step number. This is provided merely for assistance to the skilled chemist.

When reference is made to the use of a “similar” or “analogous” procedure, as it will be appreciated by those skilled in the art, such a procedure may involve minor variations, for example reaction temperature, reagent/solvent amount, reaction time, work-up conditions or chromatographic purification conditions, that will be appreciated by those skilled in the art. All final compounds were obtained as a free base, unless stated otherwise.

### Abbreviations

AcOH=acetic acid; Acetone-d6=deuterated acetone; ACN=acetonitrile; ACN-d3=deuterated acetonitrile; CDCl₃=deuterated chloroform; cHex=cyclohexane; CV=Column Volumes; DCM=dichloromethane; DIPEA=diisopropylethylamine; DMA=N,N-dimethylacetamide; DMF=dimethylformamide; DMSO=dimethyl sulfoxide; DMSO-d6=deuterated dimethyl sulfoxide; ee=enantiomeric excess; Et₂O=diethyl ether; EtOAc=ethyl acetate; eq=equivalents; FCC=flash column chromatography; h=hour/s; HATU=1-[Bis(dimethylamino)methylene]-1H-1,2,3-triazolo[4,5-b]pyridinium 3-oxide hexafluorophosphate; HCOOH=formic acid; HPLC=High Performance Liquid Chromatography; LCMS=Liquid Chromatography/Mass Spectrometry; MeOH=methyl alcohol; Me-THF=2-Methyltetrahydrofuran; min=minute/s; NMR=nuclear magnetic resonance; Pd₂(dba)₃=tris(dibenzylideneacetone)dipalladium(0); XPhos Pd G3=2-Dicyclohexylphosphino-2′,4′,6′-triisopropyl-1,1′-biphenyl) [2-(2′-amino-1,1′-biphenyl)]palladium (II) methanesulfonate; RT/rt=room temperature; RuPhos=2-Dicyclohexylphosphino-2′,6′-diisopropoxybiphenyl; SCX=strong cation exchange; SFC=supercritical fluid chromatography; STAB=sodium triacetoxyborohydride; SM=starting material; tBu=tert-Butyl; tBuOK=Potassium tert-butoxide; TCFH=chloro-N,N,N′,N′-tetramethylformamidinium hexafluorophosphate; TEA=triethylamine; TFA=trifluoroacetic acid; THF=tetrahydrofuran; TLC=Thin Layer Chromatography; t_{R}=retention time; UPLC=Ultra Performance Liquid Chromatography; TBAF=tetrabutylammonium fluoride; TBAB=Tetrabutylammonium bromide; DBU=1,8-diazabiciclo[5.4.0]undec-7-ene; CPME=cyclopentyl methyl ether; mCPBA=3-Chloroperbenzoic acid; CDI 1,1′-Carbonyldiimidazole; Xantphos=4,5-Bis(diphenylphosphino)-9,9-dimethylxanthene; MeI=methyl iodide; SEM=2-(Trimethylsilyl) ethoxymethyl; Boc=tert-Butyloxycarbonyl; Troc=2,2,2-trichloroethyl carbamate; LiOH=lithium hydroxyde.

### General Experimental Details

### NMR Characterization:

¹H NMR spectra were recorded on Bruker instrument operating at 400 MHz (proton frequency), using the stated solvent at around rt unless otherwise stated. In all cases, NMR data were consistent with the proposed structures. Characteristic chemical shifts are reported as δ values in ppm (parts-per-million). Coupling constants (J values) are given in hertz (Hz) and multiplicities are reported using the following conventional abbreviations for designation of major peaks: s=singlet, d=doublet, t=triplet, q=quartet, dd=doublet of doublets, dt=doublet of triplets, m=multiplet, br=broad, nd=not determined.

In some cases, signals NH from amide bond or amine bond (exchangeable protons) are not visible. In a few cases, some signals could be hidden or partly covered by the signal of water or under the DMSO peak or other residual solvents.

### LC/UV/MS Analytical Methods

LC/MS retention times are estimated to be affected by an experimental error of ±0.5 min.

Method 1: Agilent Zorbax column 4.6×50 mm, 3.5 μm, maintained at 40° C. Mobile phase:MeCN (0.1% formic acid) in water (0.1% formic acid), from 5% to 95% within 2 min. Flow rate: 3.0 ml/min. Wavelength: 210-400 nm DAD. Waters™ 2795/2695 separations module+Waters™ DAD+Micromass ZQ, single quadrupole LCMS.

Method 2: Waters™ Acquity UPLC HSS C18 column, 100×2.1 mm, 1.8 μm (Plus guard cartridge), maintained at 40° C. Mobile phase: MeCN (0.1% formic acid) in water (0.1% formic acid) from 5% to 95% within 5.6 min. Flow rate: 0.4 ml/min. Wavelength: 210-400 nm DAD. UPLC+Waters™ DAD+Waters™ SQD2, single quadrupole UPLCMS

Method 3: Waters™ Acquity UPLC BEH Shield RP18 column, 100×2.1 mm, 1.72 μm (Plus guard cartridge), maintained at 40° C. Mobile phase: MeCN in water+10 nM ammonium bicarbonate from 5% to 95% within 5.6 min. Flow rate: 0.4 ml/min. Wavelength: 210-400 nm DAD. UPLC+Waters™ DAD+Waters™ SQD2, single quadrupole UPLCMS

Method 4: Waters™ Sunfire C18 column, 4.6×50 mm, 3.5 μm, maintained at 40° C. Mobile phase MeCN in water+10 mM ammonium bicarbonate, from 5 to 95% within 2.5 min. Flow rate: 2.0 mL/min. Wavelength: 210-400 nm DAD. Waters™ 2795 separations module+Waters™ DAD+Micromass ZQ, single quadrupole LCMS.

Method 5: Waters Acquity QSM, Kinetex C8 column 100 mm×2.1 mm 1.7 μm, maintained at 55° C.; Mobile Phase: Eluent A (HCOONH4 0.025M pH 3), Eluent B (ACN+0.1% FA). Gradient mode: from 0 to 3 min. eluent B is increased from 1% to 30%, from 3 to 6.50 min. is increased from 30% to 50%, from 6.50 to 7.50 min. is increased from 50% to 80%, from 7.50 to 8 min is kept at 80%, from 8 to 8.10 min. is decreased from 80% to 1% and from 8.10 it is kept at 1% till the end at 10 min. Flow rate: 0.5 mL/min. Wavelength: 210-400 nm PAD. UPLC+Waters PDA+Xevo TQS MS instrument.

Method 6: Waters™ Acquity QSM, Acquity UPLC CSH C18 column 50 mm×2.1 mm 1.7 μm, maintained at 50° C.; Mobile Phase: Eluent A (HCOONH₄ 0.025M pH 3), Eluent B (ACN+0.1% FA). Gradient mode: from 0 to 5.50 min. eluent B is increased from 20% to 80%, from 5.50 to 7.50 min. is kept at 80%, from 7.50 to 8 min. is decreased from 80% to 20%, and from 8 min. it is kept at 20% till the end at 10 min. Flow rate: 0.35 mL/min. Wavelength: 210-400 nm DAD. UPLC+Waters™ PDA+Xevo TQS MS instrument.

Method 7: Acquity CSH C18 column 50 mm×2.1 mm 1.7 μm, maintained at 40° C.; Mobile Phase: Eluent B (ACN) in Eluent A (water+0.1% HCOOH) from 1% to 99.9% within 1.5 min. Flow rate: 1 mL/min. Wavelength: 210-400 nm DAD. UPLC+Waters PDA+Waters QDA.

Method 8: Kinetex® XB-C18 column, 4.6×50 mm, 2.6 μm maintained at 25° C. Mobile phase: water (0.1% formic acid) in MeCN (0.1% formic acid), from 80% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 9: Kinetex® XB-C18 column, 4.6×50 mm, 2.6 μm maintained at 25° C. Mobile phase: water (0.1% formic acid) in MeCN (0.1% formic acid), from 90% to 5% within 3.90 min; Flow rate: 1.0 ml/min; wavelength: 190-340 nm DAD. Dionex UHPLC Ultimate 3000 with DAD detector/Thermo Scientific ISQ EC mass spectrometer.

Method 10: Acquity UPLC BEH-Waters, 1.7 μm C18 (2.1×100 mm), 130 Å, maintained at 25° C. Mobile phase: water (0.1% formic acid) in MeCN (0.1% formic acid), from 80% to 5% within 2.70 min; Flow rate: 0.5 ml/min; wavelength: 254 nm. Shimadzu LCMS-2020 Single Quadrupole Liquid Chromatograph Mass Spectrometer.

Method 11: Waters Acquity UPLC BEH C18 column 2.1×100 mm, 1.7 μm, (Plus guard cartridge), maintained at 40° C. Mobile phase: MeCN (0.1% formic acid) in water (0.1% formic acid), from 5% to 95% within 8 min. Flow rate: 0.4 ml/min. Wavelength: 200-400 nm DAD, Waters Aquity PDA, separations module+Waters PDA+Micromass ZQ, single quadrapole LC-MS.

Method 12: Waters Acquity UPLC BEH C18 column 2.1×100 mm, 1.7 μm, (Plus guard cartridge), maintained at 40° C. Mobile phase: MeCN (0.1% Ammonium Hydroxide) in water (0.1% Ammonium Hydroxide), from 5% to 95% within 8 min. Flow rate: 0.4 ml/min. Wavelength: 200-400 nm DAD, Waters Acquity PDA, separations module+Waters PDA+Micromass ZQ, single quadrapole LC-MS.

Method 13: Acquity CSH C18 column 50 mm×2.1 mm 1.7 μm, maintained at 40° C.; Mobile Phase: Eluent B (ACN) in Eluent A (water+0.1% HCOOH) from 1% to 99.9% within 3.5 min. Flow rate: 1 mL/min. Wavelength: 210-400 nm DAD. UPLC+Waters PDA+Waters QDA.g

Method 14: Acquity UPLC BEH-Waters, 1.7 μm C18 (2.1×100 mm), 130 Å, maintained at 25° C. Mobile phase: water (0.1% formic acid) in MeCN (0.1% formic acid), from 80% to 5% within 3.9 min; Flow rate: 0.5 mL/min; wavelength: 220 nm and 254 nm. Shimadzu LCMS-2020 Single Quadrupole Liquid Chromatograph Mass Spectrometer.

Method 15: Phenomenex Kinetex EVO C18 HPLC column, 4.6×50 mm, 5 μm, operating at room temperature: Mobile phase: MeCN (0.1% NH₄OH) in water (0.1% NH₄OH), from 5 to 95% within 2.5 min. Flow rate: 1.5 mL/min. Wavelength: 210-400 nm DAD. Acquity H-Class UPLC with PDA detector and QDa.

Method 16: Waters Aquity UPLC HSS C18 column, 2.1×5 0 mm, 1.8 μm, maintained at 40° C.: MeCN (0.1% FA) in water (0.1% FA), from 5 to 95% within 2.5 min. Flow rate: 1.5 mL/min. Wavelength: 210-400 nm DAD. Acquity H-Class UPLC with PDA detector and QDa.

Method 17: Waters Aquity UPLC BEH C18 column 2.1×100 mm, 1.7 μm, (Plus guard cartridge), maintained at 40° C. Mobile phase: MeCN (0.1% FA) in water (0.1% FA), from 5% to 50% within 8 min. Flow rate: 0.4 mL/min. Wavelength: 200-400 nm DAD, Waters Aquity PDA, separations module+Waters PDA+Micromass ZQ, single quadrapole LCMS.

Method 18: Waters Aquity UPLC BEH C18 column 2.1×100 mm, 1.7 μm, (Plus guard cartridge), maintained at 40° C. Mobile phase: MeCN (0.1% FA) in water (0.1% FA), from 10% to 50% within 8 min. Flow rate: 0.4 mL/min. Wavelength: 200-400 nm DAD, Waters Aquity PDA, separations module+Waters PDA+Micromass ZQ, single quadrapole LCMS.

The purification of some compounds was achieved by Achiral Supercritical Fluid Chromatography (SFC) using a Waters™ Thar Prep100 preparative SFC system.

Method 19: (TORUS DEA 20×150 mm, 5 μm 10-20% MeOH (0.1% NH₄OH)/CO₂, 100 ml/min, 120 bar, 40° C.

Method 20: YMC Amylose-C MeOH, WATERS VIRIDIS 2-EP 20×250 mm, 5 μm 5-15% MeOH (0.1% NH₄OH)/CO₂, 100 mL/min, 120 bar, 40° C., DAD 235 nm.

Other compounds were purified by reverse phase HPLC using a Waters™ Fractionlynx™ preparative HPLC system or equivalent system, both in basic conditions (ACN+0.1% NH₃, H₂O+0.1% NH₃) and in acidic conditions (ACN+0.1% HCOOH, H₂O+0.1% HCOOH), wherein, in the last case, the fractions containing the required product (identified by TLC and/or LCMS analysis) were pooled and the solvent either removed under reduced pressure or lyophilised; or alternatively extracted by SCX (NH) to obtain the free base of the product, unless differently stated

Flash chromatography (FCC) was performed on Biotage® Isolera™ system or similar instruments.

For reverse phase FCC (RF FCC) the following gradient/eluent were used: gradient eluent A:eluent B from 100:0 to 0:10 in 12 CV eluent A:H₂O/ACN/HCOOH 95:5:0.1 Eluent B:H₂O/ACN/HCOOH 5:95:0.1. After purification, SCX (NH) was utilized to obtain free base of the product, unless differently stated.

All solvents were purchased from commercial sources and were used without additional purification.

### General Synthetic Procedures

### Intermediate 1: 6-(4-methylpiperazin-1-yl) imidazo[1,2-a]pyridine-3-carboxylic Acid

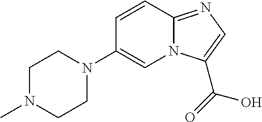 [SMILES: CN1CCN(c2ccc3ncc(C(=O)O)n3c2)CC1]

### Step 1-Methyl 6-(4-methylpiperazin-1-yl) imidazo[1,2-a]pyridine-3-carboxylate (Intermediate 2)

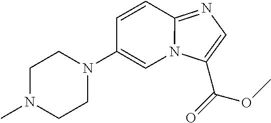

To a solution of ethyl 6-bromoimidazo[1,2-a]pyridine-3-carboxylate (807 mg, 3.00 mmol) in toluene (10 mL) Pd₂(dba)₃ (275 mg, 0.300 mmol), BINAP (560 mg, 0.900 mmol) and sodium tert-butoxide (403 mg, 4.20 mmol) were added. The reaction mixture was sparged with nitrogen then 1-methylpiperazine (0.37 mL, 3.30 mmol) was added and the reaction mixture was stirred at 100° C. for 3 h. The reaction mixture was allowed to cool to rt and filtered through a pad of Celite®, which was then washed with MeOH. The combined organic phases were concentrated in vacuo, the residue was purified by FCC (0-100% EtOAc in cHex followed by 0-100% MeOH in EtOAc). The material was taken on to the next step without further purification, as a mixture of methyl and ethyl esters.

LCMS (ESI): Method 1, t_{R}=0.65 min, m/z (M+1)=275 (Methyl ester);

LCMS (ESI): Method 1, t_{R}=0.75 min, m/z (M+1)=289 (Ethyl ester)

Analogously the following Intermediates were prepared, using the suitable amine, bromo-intermediate, the catalyst shown in the Table 6 and, in a few cases, dioxane or Me-THF as a solvent and Cs₂CO₃ as a base.

TABLE 6

| | | Product Amount |
|---|---|---|
| | Structure | (Yield), catalyst |
| N^{o} | Analytical Data | |
| Interme- diate 20 | 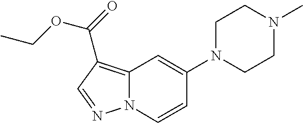 [SMILES: CCOC(=O)c1cnn2ccc(N3CCN(C)CC3)cc12] | 390 mg (73%), Palladium(II) acetate/BINAP |
| | LCMS (ESI): Method 1 min, t_{R} = 1.12 min, m/z (M + 1) = 289.1 | |
| Interme- diate 21 | 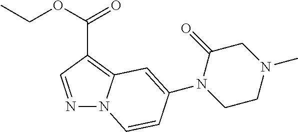 [SMILES: CCOC(=O)c1cnn2ccc(N3CCN(C)CC3=O)cc12] | 62 mg (55%), Xanthphos/Pd₂(dba)₃ |
| | LCMS (ESI): Method 10, t_{R} = 1.14 min, m/z (M + 1) = 303.15 | |
| Interme- diate 22 | 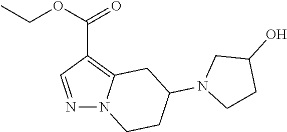 [SMILES: CCOC(=O)c1cnn2c1CC(N1CCC(O)C1)CC2] | 102 mg (quantitative), Xanthphos/Pd₂(dba)₃ |
| | LCMS (ESI): Method 10 min, t_{R} = 1.75 min, m/z (M + 1) = 276.45 | |
| Interme- diate 23 | 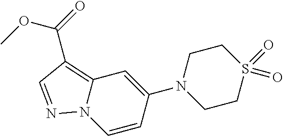 [SMILES: COC(=O)c1cnn2ccc(N3CCS(=O)(=O)CC3)cc12] | 120 mg (quantitative), Xantphos/Pd₂(dba)₃ |
| | LCMS (ESI): Method 10, t_{R} = 1.81 min, m/z (M + 1) = 324.50 | |
| Interme- diate 100 | 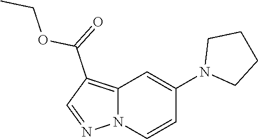 [SMILES: CCOC(=O)c1cnn2ccc(N3CCCC3)cc12] | 83 mg (42%), Xantphos/Cs₂CO₃/ Pd₂(dba)₃ |
| | LCMS (ESI): Method 7, t_{R} = 1.03 min, m/z (M + 1) = 260.21− | |
| Interme- diate 101 | 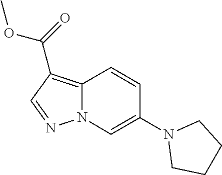 [SMILES: COC(=O)c1cnn2cc(N3CCCC3)ccc12] | 102 mg (71%), Xantphos/Cs₂CO₃/ Pd₂(dba)₃ |
| | LCMS (ESI): Method 7, t_{R} = 1.03 min, m/z (M + 1) = 246.01.50 | |
| Interme- diate 102 | 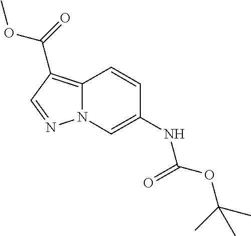 [SMILES: COC(=O)c1cnn2cc(NC(=O)OC(C)(C)C)ccc12] | 74 mg (51%), Xantphos/Me- THF/Cs₂CO₃/Pd₂(dba)₃ |
| | LCMS (ESI): Method 7, t_{R} = 1.01 min, m/z (M + 1) = 292.18 | |
| Interme- diate 103 | 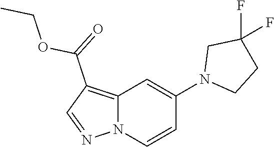 [SMILES: CCOC(=O)c1cnn2ccc(N3CCC(F)(F)C3)cc12] | 198 mg (86%), Xantphos/Me- THF/Cs₂CO₃/Pd₂(dba)₃ |
| | LCMS (ESI): Method 7, t_{R} = 1.0 min, m/z (M + 1) = 296.04 | |
| Interme- diate 104 | 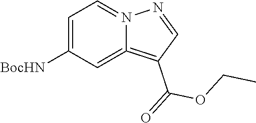 | 142 mg (83%), Xantphos/Me- THF/Cs₂CO₃/Pd₂(dba)₃ |

LCMS (ESI): Method 7, t_{R} = 1.08 min, m/z (M + 1) = 308.03

### Step 2—6-(4-methylpiperazin-1-yl) imidazo[1,2-a]pyridine-3-carboxylic Acid (Intermediate 1)

LiOH (314 mg, 13.1 mmol) in water (1 mL) was added to Intermediate 2 (1200 mg, 4.37 mmol) in THF (1 mL) at 0° C. The reaction mixture was allowed to warm to RT and stirred for 18 h. The reaction mixture was concentrated in vacuo. The residue was washed with water and the aqueous phase was adjusted to pH 7 with 1M HCl_{(aq)} and washed with DCM. The aqueous fractions were concentrated in vacuo to yield a mixture of product and inorganic salts. The material was taken on to the next step without further purification.

LCMS (ESI): Method 1, t_{R}=0.87 min, m/z (M+1)=261

Analogously the following Intermediates, as carboxylic acids or as salts, were prepared (see Table 7 below).

TABLE 7

| | | Product Amount |
|---|---|---|
| | Structure | (Yield) |
| N^{o} | Analytical Data | |
| Interme- diate 24 | 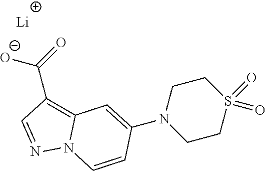 [SMILES: [Li+] ; O=C([O-])c1cnn2ccc(N3CCS(=O)(=O)CC3)cc12] | 112 mg (quantitative) |
| | LCMS (ESI): Method 10, t_{R} = 1.29 min, m/z (M + 1) = 296.20 | |
| Interme- diate 25 | 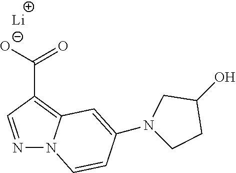 [SMILES: [Li+] ; O=C([O-])c1cnn2ccc(N3CCC(O)C3)cc12] | 92 mg (quantitative) |
| | LCMS (ESI): Method 10, t_{R} = 1.41 min, m/z (M + 1) = 247.85 | |
| Interme- diate 26 | 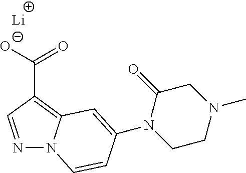 [SMILES: [Li+] ; CN1CCN(c2ccn3ncc(C(=O)[O-])c3c2)C(=O)C1] | 98 mg (96%) |
| | ¹H NMR (300 MHz, DMSO-d6) δ 8.14 (d, J = 7.5 Hz, 1H), 7.91 (s, 1H), 7.13 (s, | |
| | 1H), 6.30 (d, J = 7.5 Hz, 1H), 3.18 (d, J = 9.8 Hz, 2H), 2.90 (s, 2H), 2.59 (s, 2H), | |
| | 2.26 (s, 3H). | |
| Interme- diate 27 | 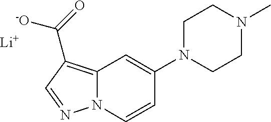 | 360 mg (quantitative) |
| | ¹H NMR (400 MHz, DMSO-d6) δ 8.32 (d, J = 7.8 Hz, 1H), 7.87 (s, 1H), 7.43 (d, | |
| | J = 2.6 Hz, 1H), 6.72 (dd, J = 2.8, 7.8 Hz, 1H), 3.21 (d, J = 5.0, 5.0 Hz, 4H), 2.49- | |
| | 2.46 (m, 4H), 2.24 (s, 3H). | |
| Interme- diate 105 | 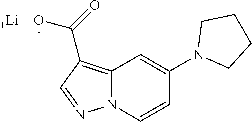 [SMILES: [Li+] ; O=C(O)c1cnn2ccc(N3CCCC3)cc12] | 77 mg (quantitative) |
| | LCMS (ESI): Method 7, t_{R} = 0.73 min, m/z (M + 1) = 232.03 | |
| Interme- diate 106 | 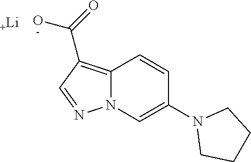 [SMILES: [Li+] ; O=C(O)c1cnn2cc(N3CCCC3)ccc12] | 99 mg (quantitative) |
| | LCMS (ESI): Method 7, t_{R} = 0.83 min, m/z (M + 1) = 231.98 | |
| Interme- diate 107 | 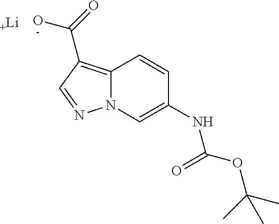 [SMILES: [Li+] ; CC(C)(C)OC(=O)Nc1ccc2c(C(=O)O)cnn2c1] | 102 mg (quantitative) |
| | LCMS (ESI): Method 7, t_{R} = 0.83 min, m/z (M + 1) = 277.99 | |
| Interme- diate 108 | 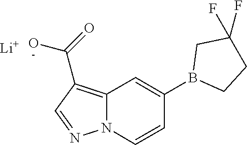 [SMILES: [Li+] ; O=C(O)c1cnn2ccc(B3CCC(F)(F)C3)cc12] | 180 mg (quantitative) |
| | LCMS (ESI): Method 7, t_{R} = 0.72 min, m/z (M + 1) = 267.98 | |
| Interme- diate 109 | 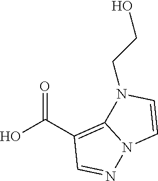 [SMILES: O=C(O)c1cnn2ccn(CCO)c12] | 150 mg, (quantitative) |
| | LCMS (ESI): Method 1, t_{R} = 0.97 min, m/z (M + 1) = 196.0 | |
| Interme- diate 110 | 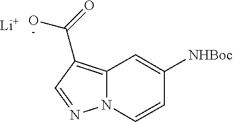 [SMILES: [Li+] ; CC(C)(C)OC(=O)Nc1ccn2ncc(C(=O)O)c2c1] | 132 mg (quantitative) |

LCMS (ESI): Method 7, t_{R} = 0.81 min, m/z (M − 1) = 276.11

### Intermediate 3: phenyl (5-(tert-butyl)isoxazol-3-yl)carbamate

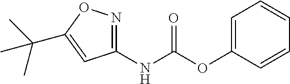 [SMILES: CC(C)(C)c1cc(NC(=O)Oc2ccccc2)no1]

To a suspension of 5-(tert-butyl) isoxazol-3-amine (321 mg, 2.29 mmol) and Na₂CO₃ (171 mg, 1.61 mmol) in EtOAc (20.00 mL), THF (4.00 mL) and water (4.00 mL) at 0° C., phenyl chloroformate (0.37 mL, 2.95 mmol) was added dropwise. The reaction mixture was stirred at 0° C. for 20 min, then warmed up to rt and stirred for 18 h. The reaction was concentrated in vacuo, EtOAc (20 mL) added and washed with water (30 mL). The organic phase was dried over a hydrophobic frit and concentrated in vacuo to give the title compound (487 mg, 1.25 mmol, 55% yield).

LCMS (ESI): Method 4, t_{R}=1.69 min, m/z (M+1)=261

Analogously the following Intermediates were prepared (see Table 8 below).

TABLE 8

| | | Product Amount |
|---|---|---|
| | Structure | (Yield) |
| N^{o} | Analytical Data | |
| Interme- diate 4 | 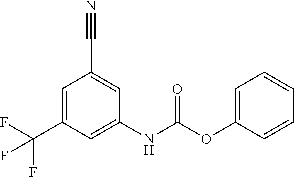 [SMILES: N#Cc1cc(NC(=O)Oc2ccccc2)cc(C(F)(F)F)c1] | 463 mg, (56%) |
| | LCMS (ESI): Method 4, t_{R} = 1.74 min, m/z (M − 1) = 305 | |
| Interme- diate 111 | 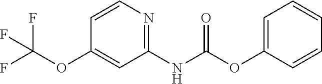 [SMILES: O=C(Nc1cc(OC(F)(F)F)ccn1)Oc1ccccc1] | 67 mg, (50%), DIPEA |
| | LCMS (ESI): Method 4, t_{R} = 1.4 min, m/z (M + 1) = 299.1 | |
| Interme- diate 112 | 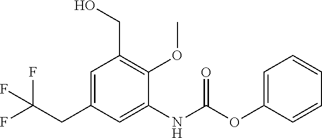 [SMILES: COc1c(CO)cc(CC(F)(F)F)cc1NC(=O)Oc1ccccc1] | 497 mg, (33%) |
| | ¹H NMR (300 MHz, Chloroform-d) δ 8.09 (s, 1H), 7.54 (s, 1H), 7.50-7.37 (m, | |
| | 2H), 7.33-7.29 (m, 1H), 7.25 (d, J = 1.3 Hz, 1H), 7.22 (dd, J = 2.0, 0.9 Hz, 1H), | |
| | 7.08-7.01 (m, 1H), 4.80 (s, 2H), 3.91 (s, 3H). | |
| Interme- diate 113 | 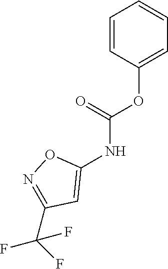 [SMILES: O=C(Nc1cc(C(F)(F)F)no1)Oc1ccccc1] | 48 mg, (11%) |

LCMS (ESI): Method 7, m/z (M + 1) = 272.04

### Intermediate 114: (3-amino-2-methoxy-5-(trifluoromethoxy)phenyl)methanol

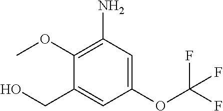 [SMILES: COc1c(N)cc(OC(F)(F)F)cc1CO]

(2-methoxy-3-nitro-5-(trifluoromethoxy)phenyl)methanol (2.64 g, 9.88 mmol) was prepared by following the procedure described in U.S. Pat. No. 10,280,145.

### Intermediate 5: 4-bromo-1-((2-(trimethylsilyl) ethoxy)methyl)-1H-pyrazolo[3,4-b]pyridine

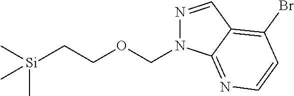 [SMILES: C[Si](C)(C)CCOCn1ncc2c(Br)ccnc21]

To a solution of 4-bromo-1H-pyrazolo[3,4-b]pyridine (1160 mg, 5.86 mmol) and/BuOK (986 mg, 8.79 mmol) in DMF (15.00 mL), 2-(trimethylsilyl) ethoxymethyl chloride (1.2 mL, 7.03 mmol) was added dropwise at 0° C. and the mixture was allowed to warm to rt and stirred overnight. The reaction mixture was quenched with sat. aq. NH₄Cl and extracted with EtOAc. The organic phase was washed with water and brine, dried over MgSO₄ and concentrated in vacuo. The residue was purified by FCC (0-60% EtOAc in cHex) to give the title compound (959 mg, 2.92 mmol, 50% yield).

LCMS (ESI): Method 4, t_{R}=1.96 min, m/z (M+1)=328.0; 330.0

¹H NMR (400 MHz, DMSO-d6) δ 8.58 (d, J=4.9 Hz, 1H), 8.40 (s, 1H), 7.72 (d, J=4.9 Hz, 1H), 5.90 (s, 2H), 3.71 (t, J=8.0 Hz, 2H), 0.93 (t, J=8.0 Hz, 2H), 0.01 (s, 9H).

Analogously the following Intermediate was prepared, using sodium hydride as a base (see Table 9).

TABLE 9

| | | Product Amount |
|---|---|---|
| | Structure | (Yield) |
| N^{o} | Analytical Data | |
| Interme- diate 28 | 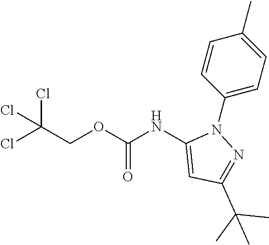 [SMILES: Cc1ccc(-n2nc(C(C)(C)C)cc2NC(=O)OCC(Cl)(Cl)Cl)cc1] | 17 mg (15%) |

LCMS (ESI): Method 4 min, t_{R} = 2.58 min, m/z (M + 1) = 329.99

### Intermediate 29: 2,2,2-trichloroethyl (3-(tert-butyl)-1-(p-tolyl)-1H-pyrazol-5-yl)carbamate

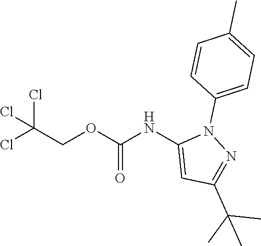 [SMILES: Cc1ccc(-n2nc(C(C)(C)C)cc2NC(=O)OCC(Cl)(Cl)Cl)cc1]

A solution of 3-(tert-butyl)-1-(p-tolyl)-1H-pyrazol-5-amine (100 mg, 0.436 mmol) and NaOH (1M aq., 1.3 mL, 1.31 mmol) in EtOAc (3 mL) was cooled in an ice/water bath, 2,2,2-trichloroethyl chloroformate (0.066 mL, 0.480 mmol) was added dropwise and the reaction mixture was stirred at rt for 1.5 h, then concentrated in vacuo to give the title compound (100 mg, 0.247 mmol, 57% yield) and it was used as crude in the next reaction.

LCMS (ESI): Method 1, t_{R}=1.86 min, m/z (M+1)=404.0, 406.0

¹H NMR (400 MHz, CDCl₃) δ 7.36-7.26 (m, 4H), 6.82 (br s, 1H), 6.41 (s, 1H), 4.81 (s, 2H), 2.41 (s, 3H), 1.34 (s, 9H).

All the following Troc carbamates were prepared by using the same procedure; sometimes DIPEA was used as a base and THE or pyridine as a solvent, starting from the suitable amine (see Table 10)

TABLE 10
IUPAC Name

2,2,2-trichloroethyl (4-(trifluoromethyl)pyridin-2-yl)carbamate
2,2,2-trichloroethyl (2-methyl-5-(trifluoromethoxy)phenyl)carbamate
2,2,2-trichloroethyl (4-(difluoromethyl)pyridin-2-yl)carbamate
2,2,2-trichloroethyl (3-(difluoromethyl)phenyl)carbamate
2,2,2-trichloroethyl (3-(difluoromethoxy)phenyl)carbamate
2,2,2-trichloroethyl (2-methoxy-5-(trifluoromethyl)phenyl)carbamate
2,2,2-trichloroethyl (3-cyano-5-(trifluoromethoxy)phenyl)carbamate
2,2,2-trichloroethyl (2-fluoro-5-(trifluoromethoxy)phenyl)carbamate
2,2,2-trichloroethyl (4-(trifluoromethoxy)pyridin-2-yl)carbamate
2,2,2-trichloroethyl (3-(hydroxymethyl)-2-methoxy-5-(trifluoromethoxy)
phenyl)carbamate
2,2,2-trichloroethyl (5-(difluoromethoxy)-2-methoxyphenyl)carbamate
2,2,2-trichloroethyl (5-(trifluoromethyl)pyridin-3-yl)carbamate
2,2,2-trichloroethyl (2-chloro-5-(trifluoromethoxy)phenyl)carbamate
2,2,2-trichloroethyl (3-(hydroxymethyl)-5-(trifluoromethyl)phenyl)
carbamate
2,2,2-trichloroethyl (3-(pentafluoro-16-sulfaneyl)phenyl)carbamate
2,2,2-trichloroethyl (5-(tert-butyl)-1-methyl-1H-pyrazol-3-yl)carbamate
2,2,2-trichloroethyl (6-(trifluoromethyl)pyrimidin-4-yl)carbamate
2,2,2-trichloroethyl (2-methoxy-5-(trifluoromethyl)pyridin-3-yl)
carbamate
2,2,2-trichloroethyl (4-chloro-3-(trifluoromethyl)phenyl)carbamate
2,2,2-trichloroethyl (2-methoxy-5-(pentafluoro-16-sulfaneyl)phenyl)
carbamate
2,2,2-trichloroethyl (4-fluoro-3-(trifluoromethoxy)phenyl)carbamate
2,2,2-trichloroethyl (3-(perfluoroethyl)phenyl)carbamate
2,2,2-trichloroethyl (3-cyano-5-(pentafluoro-16-sulfaneyl)phenyl)
carbamate
2,2,2-trichloroethyl (4-fluoro-3-(trifluoromethyl)phenyl)carbamate
2,2,2-trichloroethyl (3-fluoro-5-(trifluoromethoxy)phenyl)carbamate
2,2,2-trichloroethyl (3-((trifluoromethyl)thio)phenyl)carbamate
2,2,2-trichloroethyl (2-(oxetan-3-yloxy)-5-(trifluoromethyl)phenyl)
carbamate
2,2,2-trichloroethyl (2-methoxy-5-(trifluoromethoxy)phenyl)carbamate
2,2,2-trichloroethyl (4-(2,2,2-trifluoroethyl)pyridin-2-yl)carbamate
2,2,2-trichloroethyl (5-chloro-2-methoxypyridin-3-yl)carbamate
2,2,2-trichloroethyl (3-(tert-butyl)-1-methyl-1H-pyrazol-5-yl)carbamate
2,2,2-trichloroethyl (3-(hydroxymethyl)-5-(trifluoromethyl)phenyl)
carbamate
2,2,2-trichloroethyl (5-methoxy-2-methylphenyl)carbamate
2,2,2-trichloroethyl (2-methoxy-5-(2,2,2-trifluoroethyl)phenyl)carbamate
2,2,2-trichloroethyl (4-cyclopropylpyridin-2-yl)carbamate
2,2,2-trichloroethyl (5-cyclopropylpyridin-3-yl)carbamate
2,2,2-trichloroethyl (3-((trifluoromethyl)sulfonyl)phenyl)carbamate
2,2,2-trichloroethyl (2-methyl-5-(trifluoromethyl)pyridin-3-yl)carbamate
2,2,2-trichloroethyl (6-cyano-4-(trifluoromethyl)pyridin-2-yl)carbamate
2,2,2-trichloroethyl (2-methyl-5-(trifluoromethyl)pyridin-3-yl)carbamate
2,2,2-trichloroethyl (5-(trifluoromethoxy)pyridin-3-yl)carbamate
2,2,2-trichloroethyl (5-(1, 1-difluoroethyl)pyridin-3-yl)carbamate
2,2,2-trichloroethyl (4-(difluoromethoxy)pyridin-2-yl)carbamate
2,2,2-trichloroethyl (6-methyl-5-(trifluoromethyl)pyridin-3-yl)carbamate
2,2,2-trichloroethyl (6-(trifluoromethoxy)pyridin-3-yl)carbamate
2,2,2-trichloroethyl (5-bromopyridin-3-yl)carbamate
2,2,2-trichloroethyl (3-(1-(trifluoromethyl)cyclopropyl)phenyl)carbamate
2,2,2-trichloroethyl (5-(1,1,1-trifluoro-2-methylpropan-2-yl)pyridin-
3-yl)carbamate
2,2,2-trichloroethyl (4-bromopyridin-2-yl)carbamate
2,2,2-trichloroethyl (1-methyl-2-oxo-5-(trifluoromethyl)-1,2-
dihydropyridin-3-yl)carbamate
2,2,2-trichloroethyl (5-((trifluoromethyl)thio)pyridin-3-yl)carbamate
2,2,2-trichloroethyl (3-(tert-butyl)pyridin-5-yl)carbamate
2,2,2-trichloroethyl (5-(tert-butyl)pyridin-3-yl)carbamate
2,2,2-trichloroethyl (3-methyl-5-(trifluoromethyl)phenyl)carbamate
2,2,2-trichloroethyl (3-chloro-5-(trifluoromethyl)phenyl)carbamate
2,2,2-trifluoroethyl (2-methoxy-5-(trifluoromethyl)phenyl)carbamate
2,2,2-trichloroethyl (2-fluoro-3-methyl-5-(trifluoromethyl)phenyl)
carbamate
2,2,2-trichloroethyl (2-methyl-5-(trifluoromethyl)phenyl)carbamate
2,2,2-trichloroethyl (5-(trifluoromethyl)pyridazin-3-yl)carbamate
2,2,2-trichloroethyl (6-methoxy-4-(trifluoromethyl)pyridin-2-yl)
carbamate
2,2,2-trichloroethyl (3,5-bis(trifluoromethyl)phenyl)carbamate
2,2,2-trichloroethyl (2-methoxy-5-(trifluoromethyl)pyridin-3-yl)
carbamate
2,2,2-trichloroethyl (2-ethyl-5-(trifluoromethyl)phenyl)carbamate
2,2,2-trichloroethyl (3-fluoro-4-methyl-5-(trifluoromethyl)phenyl)
carbamate
2,2,2-trichloroethyl (5-(perfluoroethyl)pyridin-3-yl)carbamate
2,2,2-trichloroethyl (4-iodopyridin-2-yl)carbamate
2,2,2-trichloroethyl (4-methoxypyridin-2-yl)carbamate
2,2,2-trichloroethyl (3-methyl-5-(trifluoromethoxy)phenyl)carbamate
2,2,2-trichloroethyl (3-fluoro-2-methyl-5-(trifluoromethyl)phenyl)
carbamate
2,2,2-trichloroethyl (3-cyano-5-(trifluoromethyl)phenyl)carbamate
2,2,2-trichloroethyl (2-chloro-5-(trifluoromethyl)phenyl)carbamate
2,2,2-trichloroethyl (3-methoxy-5-(trifluoromethyl)phenyl)carbamate
2,2,2-trichloroethyl (2-ethoxy-5-(trifluoromethyl)phenyl)carbamate
2,2,2-trichloroethyl (2-methyl-6-(trifluoromethyl)pyrimidin-4-yl)
carbamate
2,2,2-trichloroethyl (4-(trifluoromethyl)thiophen-2-yl)carbamate
2,2,2-trichloroethyl (6-methyl-4-(trifluoromethyl)pyridin-2-yl)carbamate
2,2,2-trichloroethyl (5-methyl-4-(trifluoromethyl)pyridin-2-yl)carbamate
2,2,2-trichloroethyl (4-(perfluoroethyl)pyridin-2-yl)carbamate
2,2,2-trichloroethyl (6-bromo-4-(trifluoromethyl)pyridin-2-yl)carbamate
2,2,2-trichloroethyl (2-ethyl-5-(trifluoromethyl)pyridin-3-yl)carbamate
2,2,2-trichloroethyl (3-(hydroxymethyl)-2-methoxy-5-(trifluoromethyl)
phenyl)carbamate
2,2,2-trichloroethyl (5-(1,1,1-trifluoro-2-methylpropan-2-yl)isoxazol-3-
yl)carbamate
2,2,2-trichloroethyl (5-(trifluoromethyl)pyridazin-3-yl)carbamate

### Intermediate 30: Ethyl 1-(oxetan-3-yl)-1H-imidazo[1,2-b]pyrazole-7-carboxylate

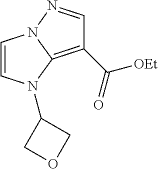 [SMILES: CCOC(=O)c1cnn2ccn(C3COC3)c12]

To a solution of ethyl 1H-imidazo[1,2-b]pyrazole-7-carboxylate (150 mg, 0.837 mmol) and 3-iodooxetane (0.22 mL, 2.51 mmol) in DMF (3.5 mL), stirring under nitrogen, potassium carbonate (231 mg, 1.67 mmol). was added in one portion. The reaction was stirred at 50° C. for 18 h. The reaction was allowed to cool down to rt and diluted with EtOAc. The organic layer was washed with saturated aqueous NaHCO₃ solution, brine, dried over MgSO₄, filtered and solvent removed in vacuo. The crude material was purified by FCC (0-100% 3:1 EtOAc:EtOH in cHex) to yield the title compound (126 mg, 0.536 mmol, 64% yield).

LCMS (ESI): Method 4, t_{R}=1.26 min, m/z (M+1)=236.2

Analogously to Intermediate 30, the following intermediates were prepared (see Table 11).

TABLE 11

| | | Product Amount |
|---|---|---|
| | Structure | (Yield) |
| N^{o} | Analytical Data | |
| Interme- diate 31 | 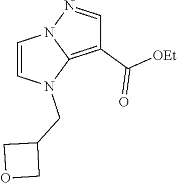 [SMILES: CCOC(=O)c1cnn2ccn(CC3COC3)c12] | 174 mg (83%) |
| | LCMS (ESI): Method 4 min, t_{R} = 1.24 min, m/z (M + 1) = 250.2 | |
| Interme- diate 115 | 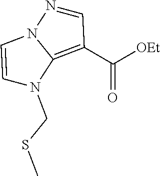 [SMILES: CCOC(=O)c1cnn2ccn(CSC)c12] | 40 mg, (20%) |
| | LCMS (ESI): Method 4, t_{R} = 1.44 min, m/z (M + 1) = 240.1 | |
| Interme- diate 116 | 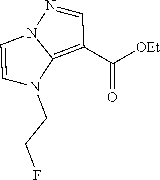 [SMILES: CCOC(=O)c1cnn2ccn(CCF)c12] | 156 mg (83%) |
| | LCMS (ESI): Method 4, t_{R} = 1.38 min, m/z (M + 1) = 226.2 | |
| Interme- diate 117 | 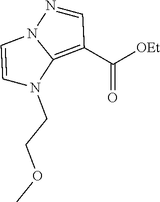 [SMILES: CCOC(=O)c1cnn2ccn(CCOC)c12] | 171 mg (86%) |
| | LCMS (ESI): Method 4, t_{R} = 1.37 min, m/z (M + 1) = 238.2 | |
| Interme- diate 118 | 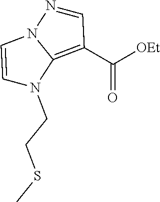 [SMILES: CCOC(=O)c1cnn2ccn(CCSC)c12] | 140 mg (66%) |
| | LCMS (ESI): Method 4, t_{R} = 1.48 min, m/z (M + 1) = 254.2 | |
| Interme- diate 119 | 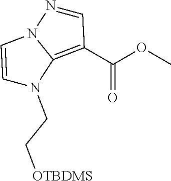 [SMILES: COC(=O)c1cnn2ccn(CCO[Si](C)(C)C(C)(C)C)c12] | 250 mg, (64%) |
| | LCMS (ESI): Method 1, t_{R} = 1.84 min, m/z (M + 1) = 324.1 | |
| Interme- diate 175 | 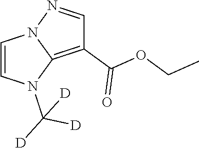 [SMILES: [2H]C([2H])([2H])n1ccn2ncc(C(=O)OCC)c12] | 180 mg, (82%) Sodium hydride was used as a base |
| | LCMS (ESI): Method 15, t_{R} = 1.34 min, m/z (M + 1) =197 | |
| Interme- diate 176 | 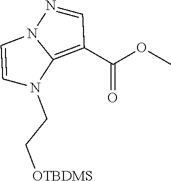 [SMILES: COC(=O)c1cnn2ccn(CCO[Si](C)(C)C(C)(C)C)c12] | 250 mg, (64%) |

LCMS (ESI): Method 1, t_{R} = 1.84 min, m/z (M + 1) = 324

### Intermediate 32: 1-(oxetan-3-yl)-1H-imidazo[1,2-b]pyrazole-7-carboxylic Acid

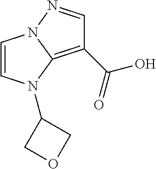 [SMILES: O=C(O)c1cnn2ccn(C3COC3)c12]

To a solution of ethyl 1-(oxetan-3-yl)-1H-imidazo[1,2-b]pyrazole-7-carboxylate (120 mg, 0.51 mmol) in THF (10 mL) sodium hydroxide (204 mg, 5.10 mmol) and MeOH (5.0 mL) were added and the mixture was heated to 50° C. for 24 h. The reaction mixture was allowed to cool down to rt, pH reduced to about 2 with 2 N HCl aqueous solution and extracted with EtOAc. Combined organic layers were washed with brine, dried over MgSO₄, filtered and solvents removed in vacuo. The title compound (100 mg, 0.48 mmol, 95% yield) was used in the next step without further purification.

LCMS (ESI): Method 1 min, t_{R}=1.02 min, m/z (M+1)=208.0

The following compounds were prepared according to the same synthetic procedure (table 12).

TABLE 12

| | | Product Amount |
|---|---|---|
| | Structure | (Yield) |
| N^{o} | Analytical Data | |
| Interme- diate 33 | 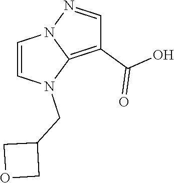 [SMILES: O=C(O)c1cnn2ccn(CC3COC3)c12] | 150 mg (57%) |
| | LCMS (ESI): Method 1 min, t_{R} = 1.04 min, m/z (M + 1) = 222.0 | |
| Interme- diate 120 | 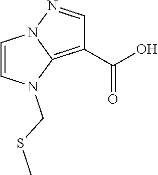 | 35 mg, (99%) |
| | LCMS (ESI): Method 4, t_{R} = 0.58 min, m/z (M + 1) = 212.1 | |
| Interme- diate 121 | 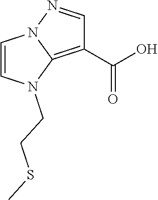 | 125 mg, (quantitative) |
| | LCMS (ESI): Method 4, t_{R} = 0.74 min, m/z (M + 1) = 226.1 | |
| Interme- diate 122 | 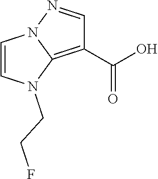 [SMILES: O=C(O)c1cnn2ccn(CCF)c12] | 136 mg, (quantitative) |
| | LCMS (ESI): Method 1, t_{R} = 1.07 min, m/z (M + 1) = 198.0 | |
| Interme- diate 123 | 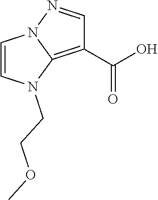 | 150 mg, (99%) |

LCMS (ESI): Method 1, t_{R} = 1.07 min, m/z (M + 1) = 210.0

### Intermediate 34: methyl 6-((oxetan-3-ylamino)methyl) pyrazolo[1,5-a]pyridine-3-carboxylate

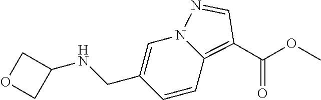 [SMILES: COC(=O)c1cnn2cc(CNC3COC3)ccc12]

To a solution of methyl 6-formylpyrazolo[1,5-a]pyridine-3-carboxylate (85 mg, 0.416 mmol) and oxetan-3-amine (0.037 mL, 0.520 mmol) in DCM (2 mL) AcOH (20 μL) was added; after 10 min NaBH(OAc)₃ (176 mg, 0.833 mmol) was added and the mixture was stirred at rt for 3 h. The reaction mixture was then diluted with DCM and washed with NaHCO₃ and water. The organic phase was dried over MgSO₄ and concentrated in vacuo to give the title compound (95 mg, 0.364 mmol, 87% yield).

LCMS (ESI): Method 7, t_{R}=1.01 min, m/z (M+1)=262.0

### Intermediate 35: lithium 6-((oxetan-3-ylamino)methyl) pyrazolo[1,5-a]pyridine-3-carboxylate

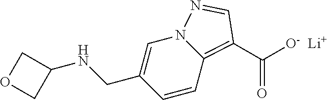 [SMILES: O=C([O-])c1cnn2cc(CNC3COC3)ccc12 ; [Li+]]

Intermediate 35 was prepared by following the procedure for Intermediate 1, step 2, to give the title compound (66 mg, 0.268 mmol, quantitative).

LCMS (ESI): Method 4 min, t_{R}=0.60 min, m/z (M+1)=248.2

### Intermediate 36: 4-(2,2,2-trifluoroethyl)pyridin-2-amine

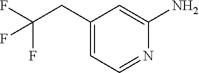 [SMILES: Nc1cc(CC(F)(F)F)ccn1]

### Step 1—2-chloro-4-(2,2,2-trifluoroethyl)pyridine (Intermediate 37)

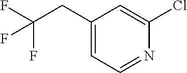 [SMILES: FC(F)(F)Cc1ccnc(Cl)c1]

To a solution of 2-chloro-isonicotinaldehyde (250 mg, 1.77 mmol) in MeOH (2.0 mL) hydrazine monohydrate (0.10 mL, 2.12 mmol) was added and the reaction stirred at rt for 1 h under nitrogen. The solvent was removed in vacuo and 1-(trifluoromethyl)-123-benzo[d][1,2]iodaoxol-3 (1H)-one (Togni reagent II, 1.023 g, 1.94 mmol) was added to the solid crude. The vial containing the two solids and a stirrer bar was subjected to vacuum/nitrogen cycles before DMSO (2.0 mL) and TFA (0.14 mL, 1.77 mmol) were added and the reaction mixture heated to 50° C. for 72 h. The reaction was allowed to cool back down to rt and it was diluted with water, extracted with Et₂O and organic layer was washed with water and brine, and then dried over MgSO₄. The organic layers were concentrated in vacuo. The residue was purified by FCC (0-50% EtOAc in cHex) to provide title compound (60 mg, 0.307 mmol, 17% yield).

LCMS (ESI): Method 1 min, t_{R}=1.57 min, m/z (M+1)=196.0, 198.0

### Step 2—4-(2,2,2-trifluoroethyl)pyridin-2-amine (Intermediate 36)

Intermediate 37 (60 mg, 0.307 mmol), tert-butyl carbamate (93 mg, 0.798 mmol), caesium carbonate (130 mg, 0.399 mmol), Xantphos (30 mg, 0.0522 mmol), Pd₂(dba)₃ (25 mg, 0.0276 mmol) and CPME (3.0 mL) were added to a reaction vessel and the mixture sparged with nitrogen for 20 minutes. The vessel was sealed and the reaction mixture was heated at 130° C. for 18 h. The reaction was allowed to cool down to rt and filtered through Celite® washing with EtOAc. Solvents were removed in vacuo. The crude mixture was redissolved in DCM (3.0 mL) and TFA (1.0 mL) then the reaction was stirred for 3 h. The reaction was concentrated to dryness and the crude residue was dissolved in MeOH and passed through an SCX Isolute column, eluting with 7 N methanolic NH₃. The ammonia solution was evaporated at reduced pressure and the title compound was used in the next step without further purification (44 mg, 31%).

LCMS (ESI): Method 1 min, t_{R}=1.32 min, m/z (M+1)=177.1

### Intermediate 124: lithium 2-(2-methoxyethyl)-1-methyl-1H-imidazo[1,2-b]pyrazole-7-carboxylate

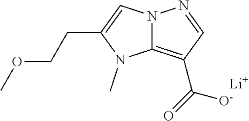 [SMILES: COCCc1cn2ncc(C(=O)[O-])c2n1C ; [Li+]]

### Step 1: ethyl 2-(2-methoxyethyl)-1H-imidazo[1,2-b]pyrazole-7-carboxylate (Intermediate 125)

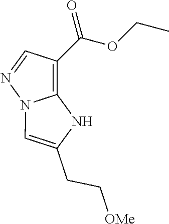 [SMILES: CCOC(=O)c1cnn2cc(CCOC)[nH]c12]

Ethyl 5-amino-1H-pyrazole-4-carboxylate (1.48 g, 9.5 mmol) was dissolved in dry ACN (20 mL), then DIPEA (1.28 ml, 7.3 mmol), tetrabutylammonium bromide (2.36 g, 7.3 mmol) and 1-chloro-4-methoxybutan-2-one (1 g, 7.3 mmol) were added and the reaction was stirred overnight at 90° C. The solvent was removed and the crude was purified by RF FCC affording title compound (0.57 g, 2.4 mmol, 33% yield).

¹H NMR (600 MHz, DMSO-d6) δ ppm 11.88 (1H, br s), 7.82 (1H, s), 7.47 (1H, s), 4.21 (2H, q, J=7.09 Hz), 3.60 (2H, t, J=6.75 Hz), 3.26 (3H, s), 2.85 (2H, m), 1.28 (3H, t, J=7.12 Hz)

### Step 2: ethyl 2-(2-methoxyethyl)-1-methyl-1H-imidazo[1,2-b]pyrazole-7-carboxylate (Intermediate 126)

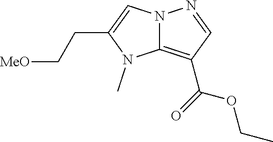 [SMILES: CCOC(=O)c1cnn2cc(CCOC)n(C)c12]

To a solution of Intermediate 125 (380 mg, 1.6 mmol), MeI (0.10 ml, 1.60 mmol) and NaH 95% w/w (40.5 mg, 1.60 mmol) were added and the mixture was stirred overnight at 50° C. The solvent was removed and the crude was purified by RF FCC affording title compound (50 mg, 0.20 mmol, 12% yield).

LCMS (ESI): Method 7, t_{R}=0.8 min, m/z (M+1)=252.0

### Step 3: lithium 2-(2-methoxyethyl)-1-methyl-1H-imidazo[1,2-b]pyrazole-7-carboxylate (Intermediate 124)

Intermediate 124 was prepared by following the procedure for Intermediate 1, step 2, to give the title compound (45 mg, 98% yield).

LCMS (ESI): Method 7, t_{R}=0.54 min, m/z (M+1)=224

### Intermediate 127: lithium 6-(1-methyl-1H-pyrazol-4-yl) pyrazolo[1,5-a]pyridine-3-carboxylate

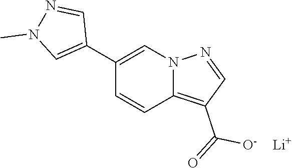 [SMILES: Cn1cc(-c2ccc3c(C(=O)[O-])cnn3c2)cn1 ; [Li+]]

### Step 1: methyl 6-(1-methyl-1H-pyrazol-4-yl) pyrazolo[1,5-a]pyridine-3-carboxylate (Intermediate 128)

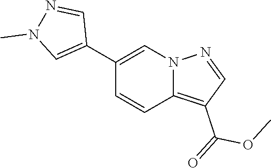

To a suspension of methyl 6-bromopyrazolo[1,5-a]pyridine-3-carboxylate (185 mg, 0.725 mmol) and (1-methylpyrazol-4-yl) boronic acid (137 mg, 1.088 mmol) in Me-THF (1.5 mL), Potassium phosphate tribasic (385 mg, 1.813 mmol) was added. After applying three N₂/vacuum cycles XPhos Pd G3 (61.4 mg, 0.073 mmol) was added and the vial was sealed. The reaction was heated at 75° C. for 12 h. EtOAc and brine were added and the organic layer was concentrated under vacuum. The crude was purified by FCC (Sfar NH, gradient of EtOAc/EtOH 3:1 in heptane from 0% to 60%) to afford title compound (61 mg, 0.24 mmol, 33% yield)

LCMS (ESI): Method 7, t_{R}=0.77 min, m/z (M+1)=256.99

### Step 2: lithium 6-(1-methyl-1H-pyrazol-4-yl) pyrazolo[1,5-a]pyridine-3-carboxylate (Intermediate 127)

Intermediate 127 was prepared by following the procedure for Intermediate 1, step 2 to give the title compound (59 mg, 100% yield).

LCMS (ESI): Method 7, t_{R}=0.54 min, m/z (M+1)=224

### Intermediate 177: 4-(perfluoroethyl)pyridin-2-amine

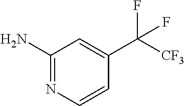 [SMILES: Nc1cc(C(F)(F)C(F)(F)F)ccn1]

In a microwave vial, 2-chloro-4-(perfluoroethyl)pyridine (250 mg, 1.08 mmol) was suspended in 35% aqueous ammonia (4.0 mL, 72.3 mmol). The vial was capped and heated in a microwave to 150° C. for 8 h, then the contents were transferred in a separating funnel and diluted with water and EtOAc. Phases were separated and the aqueous layer was extracted with EtOAc. Combined organics were washed with brine, dried over MgSO₄, filtered and solvents removed in vacuo to yield the title compound (450 mg, 2.54 mmol, quantitative) which was used without further purification.

LCMS (ESI): Method 16, t_{R}=1.50 min, m/z (M+1)=213.1

### Intermediate 178: (3-amino-2-methoxy-5-(trifluoromethyl)phenyl)methanol

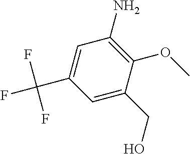

Intermediate 178 was prepared by following the procedure described in WO2017038873.

### Intermediate 179: (3-amino-2-methoxy-5-(trifluoromethoxy)phenyl)methanol

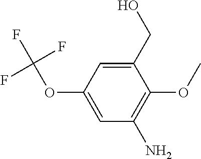 [SMILES: COc1c(N)cc(OC(F)(F)F)cc1CO]

Intermediate 179, was prepared by following the procedure described in WO2017038873.

### Example 1: 1-(2-(imidazo[1,2-a]pyridine-3-carbonyl)-2-azaspiro[3.3]heptan-6-yl)-3-(3-(trifluoromethyl)phenyl)urea

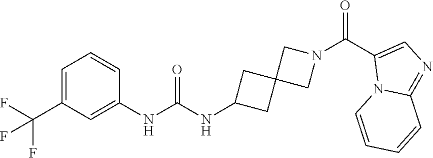 [SMILES: O=C(Nc1cccc(C(F)(F)F)c1)NC1CC2(C1)CN(C(=O)c1cnc3ccccn13)C2]

### Example 1

### Step 1—tert-butyl 6-(3-(3-(trifluoromethyl)phenyl) ureido)-2-azaspiro[3.3]heptane-2-carboxylate (Intermediate 6)

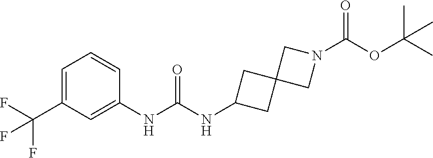 [SMILES: CC(C)(C)OC(=O)N1CC2(CC(NC(=O)Nc3cccc(C(F)(F)F)c3)C2)C1]

To a solution of tert-butyl 6-amino-2-azaspiro[3.3]heptane-2-carboxylate (500 mg, 2.36 mmol) in DCM (15.00 mL) cooled to 0° C., 1-isocyanato-3-(trifluoromethyl)benzene (0.32 mL, 2.36 mmol) was added and the reaction mixture was warmed to rt after 5 min and stirred for 2 h. It was then concentrated in vacuo to give the title compound (1 g, 2.50 mmol, quantitative yield) as a clear oil.

LCMS (ESI): Method 1, t_{R}=1.70 min, m/z (M+1-tBu)=344.0

¹H NMR (400 MHz, DMSO-d6) δ 8.65 (s, 1H), 7.86 (s, 1H), 7.44-7.34 (m, 2H), 7.14 (d, J=7.5 Hz, 1H), 6.45 (d, J=7.5 Hz, 1H), 3.97-3.85 (m, 1H), 3.81 (s, 2H), 3.70 (s, 2H), 2.42-2.35 (m, 2H), 2.02-1.91 (m, 2H), 1.29 (s, 9H).

The following Intermediates were prepared according to the same synthetic procedure, starting from commercially available compounds or previously described intermediates (see Table 13).

TABLE 13

| | | Product Amount |
|---|---|---|
| | Structure | (Yield) |
| N^{o} | Analytical Data | |
| Interme- diate 38 | 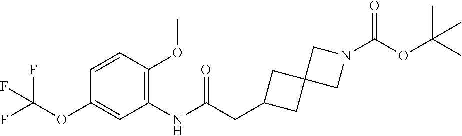 [SMILES: COc1ccc(OC(F)(F)F)cc1NC(=O)CC1CC2(C1)CN(C(=O)OC(C)(C)C)C2] | 340 mg (65%) |
| | LCMS (ESI): Method 1, t_{R} = 1.84 min, m/z (M + 1 − tBu) = 389.1 | |
| Interme- diate 39 | 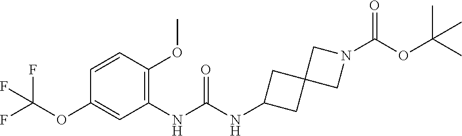 [SMILES: COc1ccc(OC(F)(F)F)cc1NC(=O)NC1CC2(C1)CN(C(=O)OC(C)(C)C)C2] | 882 mg (93%) |
| | LCMS (ESI): Method 1, t_{R} = 1.76 min, m/z (M + 1 − tBu) = 390.1 | |
| Interme- diate 40 | 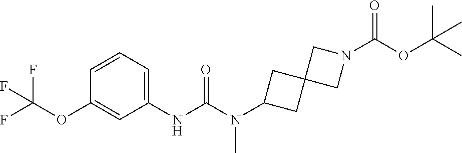 [SMILES: CN(C(=O)Nc1cccc(OC(F)(F)F)c1)C1CC2(C1)CN(C(=O)OC(C)(C)C)C2] | 130 mg (quantitative) |
| | LCMS (ESI): Method 1, t_{R} = 1.72 min, m/z (M + 1 − tBu) = 372.1 | |
| Interme- diate 41 | 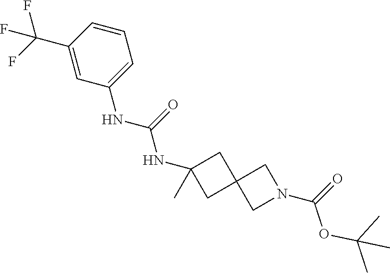 [SMILES: CC1(NC(=O)Nc2cccc(C(F)(F)F)c2)CC2(CN(C(=O)OC(C)(C)C)C2)C1] | 680 mg (93%) |
| | ¹H NMR (400 MHz, CDCl₃) δ 7.59 (s, 1H), 7.51 (d, J = 7.2 Hz, 1H), 7.37 | |
| | (dd, J = 8.0, 8.0 Hz, 1H), 7.25-7.23 (m, 1H), 3.95 (d, J = 5.5 Hz, 4H), 2.55- | |
| | 2.53 (m, 2H), 2.26 (d, J = 13.2 Hz, 2H), 1.44 (s, 9H), 1.41 (s, 3H). | |
| Interme- diate 42 | 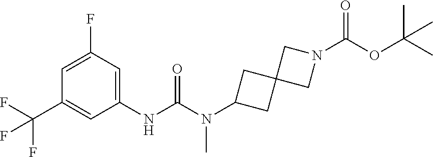 [SMILES: CN(C(=O)Nc1cc(F)cc(C(F)(F)F)c1)C1CC2(C1)CN(C(=O)OC(C)(C)C)C2] | 275 mg (87%) |
| | LCMS (ESI): Method 1, t_{R} = 1.79 min, m/z (M − 1) = 430.3 | |
| Interme- diate 43 | 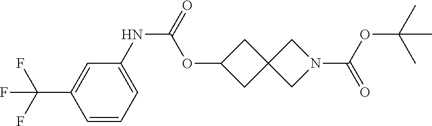 [SMILES: CC(C)(C)OC(=O)N1CC2(CC(OC(=O)Nc3cccc(C(F)(F)F)c3)C2)C1] | 1 g (53%) |

LCMS (ESI): Method 7, t_{R} = 1.30 min, m/z (M + 1 − tBu) = 345.0

### Step 2—1-(2-azaspiro[3.3]heptan-6-yl)-3-(3-(trifluoromethyl)phenyl)urea (Intermediate 7, compound IVa)

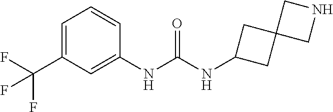 [SMILES: O=C(Nc1cccc(C(F)(F)F)c1)NC1CC2(CNC2)C1]

A solution of Intermediate 6 (900 mg, 2.25 mmol) and TFA (2.6 mL, 33.8 mmol) in DCM (15.00 mL) was stirred at rt for 1 h, then concentrated in vacuo. The residue was loaded onto an Isolute SCX cartridge, washed with 1:1 DCM:MeOH (50 mL) and 100% MeOH (20 mL), and released with 1:1 7 N NH₃ in MeOH:DCM (50 mL) and 100% 7 N NH₃ in MeOH (20 mL). The ammonia eluent was concentrated in vacuo to give the title compound (550 mg, 1.84 mmol, 82% yield).

LCMS (ESI): Method 1, t_{R}=1.29 min, m/z (M+1)=300.0

¹H NMR (400 MHz, CDCl₃) δ 7.60-7.55 (m, 1H), 7.53-7.49 (m, 1H), 7.36 (t, J=8.0 Hz, 1H), 7.28-7.23 (m, 1H), 7.17 (s, 1H), 5.24 (d, J=7.3 Hz, 1H), 4.15-4.05 (m, 1H), 3.62 (s, 2H), 3.52 (s, 2H), 2.63-2.56 (m, 2H), 1.94-1.83 (m, 2H).

The following compounds were prepared according to the same synthetic procedure of Intermediate 7 (see Table 14).

TABLE 14

| | | Product Amount |
|---|---|---|
| | Structure | (Yield) |
| N^{o} | Analytical Data | |
| Example 218 | 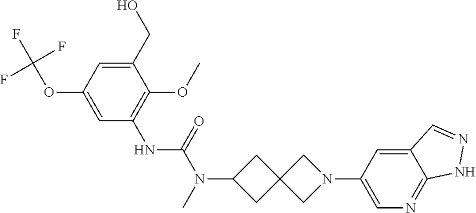 [SMILES: COc1c(CO)cc(OC(F)(F)F)cc1NC(=O)N(C)C1CC2(C1)CN(c1cnc3[nH]ncc3c1)C2] | 5 mg (22%) |
| | LCMS (ESI): Method 9, t_{R} = 3.08 min, m/z (M + 1) = 507.21 | |
| | ¹H NMR (400 MHz, DMSO-d6) δ 13.29 (s, 1H), 7.95 (d, J = 2.6 Hz, 1H), | |
| | 7.92 (s, 1H), 7.83-7.79 (m, 1H), 7.65 (s, 1H), 7.11 (d, J = 2.6 Hz, 1H), | |
| | 7.02 (dd, J = 3.0, 1.3 Hz, 1H), 5.36 (t, J = 5.6 Hz, 1H), 4.57 (d, J = 5.0 Hz, | |
| | 3H), 3.96 (s, 2H), 3.84 (s, 2H), 3.73 (s, 3H), 2.93 (s, 3H), 2.47-2.40 (m, | |
| | 4H). | |
| Example 244 | 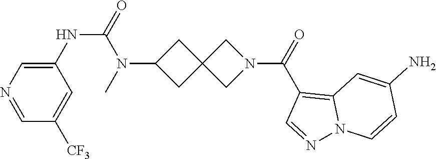 [SMILES: CN(C(=O)Nc1cncc(C(F)(F)F)c1)C1CC2(C1)CN(C(=O)c1cnn3ccc(N)cc13)C2] | 10 mg (24%) |
| | LCMS (ESI): Method 5, t_{R} = 4.63 min, m/z (M + 1) = 474.40 | |
| | ¹H NMR (400 MHz, DMSO-d6) δ ppm 8.94 (1 H, s), 8.85 (1 H, s), 8.51 (1 | |
| | H, s), 8.34 (1 H, d, J = 7.45 Hz), 8.30 (1 H, s), 7.94 (1 H, s), 7.11 (1 H, d, | |
| | J = 2.41 Hz), 6.46 (1 H, dd, J = 7.34, 2.30 Hz), 6.10 (2 H, s), 4.57 (1 H, s), | |
| | 4.15 (4 H, m), 2.92 (3 H, s), 2.41 (4 H, m) | |
| Interme- diate 44 | 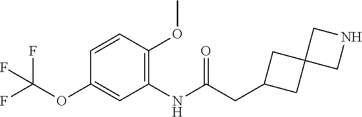 [SMILES: COc1ccc(OC(F)(F)F)cc1NC(=O)CC1CC2(CNC2)C1] | 210 mg (80%) |
| | LCMS (ESI): Method 1, t_{R} = 1.27 min, m/z (M + 1) = 345.1 | |
| Interme- diate 45 | 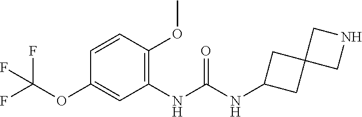 [SMILES: COc1ccc(OC(F)(F)F)cc1NC(=O)NC1CC2(CNC2)C1] | 450 mg (66%) |
| | LCMS (ESI): Method 1, t_{R} = 1.32 min, m/z (M + 1) = 346.0 | |
| Interme- diate 46 | 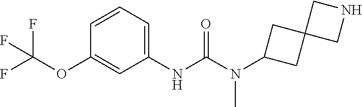 [SMILES: CN(C(=O)Nc1cccc(OC(F)(F)F)c1)C1CC2(CNC2)C1] | 60 mg (65%) |
| | LCMS (ESI): Method 1, t_{R} = 1.30 min, m/z (M +1) = 328.0 | |
| Interme- diate 47 | 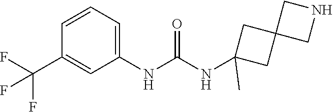 [SMILES: CC1(NC(=O)Nc2cccc(C(F)(F)F)c2)CC2(CNC2)C1] | 490 mg (95%) |
| | ¹H NMR (400 MHz, DMSO-d6) δ 8.77 (s, 1H), 7.97 (s, 1H), 7.43 (d, | |
| | J = 5.0 Hz, 2H), 7.19 (d, J = 4.0 Hz, 1H), 6.51 (s, 1H), 3.52 (s, 2H), 3.45 (s, | |
| | 2H), 2.39 (d, J = 12.8 Hz, 2H), 2.12 (d, J = 12.8 Hz, 2H), 1.30 (s, 3H). | |
| Interme- diate 48 | 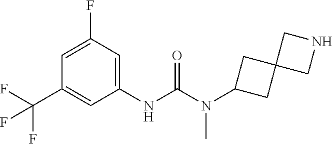 [SMILES: CN(C(=O)Nc1cc(F)cc(C(F)(F)F)c1)C1CC2(CNC2)C1] | 170 mg (82%) |
| | LCMS (ESI): Method 1, t_{R} = 1.30 min, m/z (M + 1) = 332.0 | |
| Interme- diate 49 | 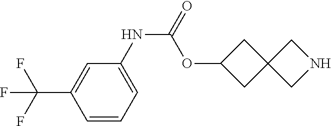 [SMILES: O=C(Nc1cccc(C(F)(F)F)c1)OC1CC2(CNC2)C1] | 87 mg (97%) |

LCMS (ESI): Method 7, t_{R} = 0.58 min, m/z (M + 1) = 301.0

### Step 3—1-(2-(imidazo[1,2-a]pyridine-3-carbonyl)-2-azaspiro[3.3]heptan-6-yl)-3-(3-(trifluoromethyl)phenyl)urea (Example 1)

To a solution of Intermediate 7 (75 mg, 0.251 mmol, 1.00 eq), imidazo[1,2-a]pyridine-3-carboxylic acid (41 mg, 0.251 mmol) and DIPEA (0.13 mL, 0.752 mmol) in DMF (2.00 mL), HATU (124 mg, 0.326 mmol) was added and the reaction mixture was stirred at rt overnight. It was then diluted with EtOAc, washed with K₂CO₃ (semi sat. aq.) and brine. The organic phase was dried MgSO₄ and concentrated in vacuo. The residue was purified by achiral SFC (method 19) to give the title compound (3.0 mg, 6.70 μmol, 3% yield).

LCMS (ESI): Method 2, t_{R}=3.41 min, m/z (M+1)=444.2

¹H NMR (400 MHz, DMSO-d6) δ 9.45 (d, J=6.8 Hz, 1H), 8.84 (s, 1H), 8.06 (s, 1H), 7.94 (s, 1H), 7.74 (d, J=8.8 Hz, 1H), 7.53-7.41 (m, 3H), 7.24-7.20 (m, 1H), 7.15-7.11 (m, 1H), 6.67 (d, J=7.6 Hz, 1H), 4.58-4.03 (m, 5H), 2.60-2.52 (m, 2H), 2.20-2.13 (m, 2H).

The following Examples were prepared analogously to what described for Example 1, step 3, by starting from the suitable, commercially available carboxylic acid intermediate or, alternatively, previously described acid intermediate or corresponding lithium salt (see Table 15).

TABLE 15

| | | Product Amount |
|---|---|---|
| | Structure | (Yield) |
| N | Analytical Data | |
| Example 2 | 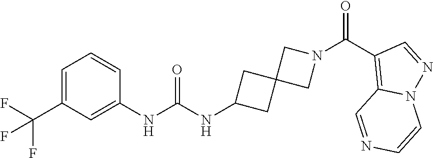 [SMILES: O=C(Nc1cccc(C(F)(F)F)c1)NC1CC2(C1)CN(C(=O)c1cnn3ccncc13)C2] | 17 mg (15%) |
| | LCMS (ESI): Method 2, t_{R} = 3.83 min, m/z (M + 1) = 445.2 | |
| | ¹H NMR (400 MHz, DMSO-d6) δ 9.55 (d, J = 1.5 Hz, 1H), 8.91 (dd, J = 1.3, | |
| | 4.5 Hz, 1H), 8.80-8.76 (m, 1H), 8.44 (s, 1H), 8.11 (d, J = 4.8 Hz, 1H), | |
| | 7.95-7.93 (m, 1H), 7.53-7.41 (m, 2H), 7.22 (d, J = 7.3 Hz, 1H), 6.61 (d, | |
| | J = 7.3 Hz, 1H), 4.59-4.47 (m, 2H), 4.16-4.04 (m, 3H), 2.60-2.53 (m, | |
| | 2H), 2.20-2.13 (m, 2H) | |
| Example 3 | 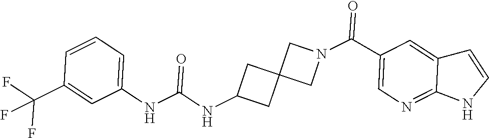 [SMILES: O=C(Nc1cccc(C(F)(F)F)c1)NC1CC2(C1)CN(C(=O)c1cnc3[nH]ccc3c1)C2] | 6.2 mg (4.2%) |
| | LCMS (ESI): method 2, t_{R} = 3.99 min, m/z (M + 1) = 444.3 | |
| | ¹H NMR (400 MHz, DMSO-d6) δ 11.90 (s, 1H), 8.82 (s, 1H), 8.50 (s, | |
| | 1H), 8.22 (s, 1H), 7.93 (s, 1H), 7.56 (t, J = 2.8 Hz, 1H), 7.50 (d, J = 8.6 Hz, | |
| | 1H), 7.43 (t, J = 7.8 Hz, 1H), 7.21 (d, J = 7.6 Hz, 1H), 6.65 (d, J = 7.6 Hz, | |
| | 1H), 6.57-6.53 (m, 1H), 4.45-4.33 (m, 2H), 4.18-3.96 (m, 3H), 2.57- | |
| | 2.52 (m, 2H), 2.12 (t, J = 9.6 Hz, 2H) | |
| Example 4 | 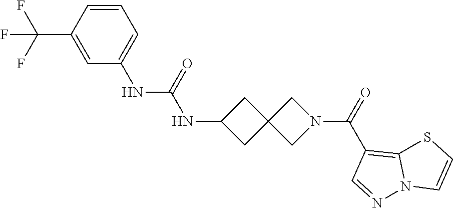 [SMILES: O=C(Nc1cccc(C(F)(F)F)c1)NC1CC2(C1)CN(C(=O)c1cnn3ccsc13)C2] | 38 mg (25%) |
| | LCMS (ESI): method 2, t_{R} = 4.10 min, m/z (M + 1) = 450.3 | |
| | ¹H NMR (400 MHz, DMSO-d6) δ 8.76 (s, 1H), 8.31 (d, J = 4.3 Hz, 1H), | |
| | 8.07 (s, 1H), 7.94 (s, 1H), 7.53-7.49 (m, 2H), 7.44 (t, J = 8.0 Hz, 1H), 7.24- | |
| | 7.20 (m, 1H), 6.61-6.57 (m, 1H), 4.48 (d, J = 46.7 Hz, 2H), 4.13-3.98 | |
| | (m, 3H), 2.59-2.53 (m, 2H), 2.19-2.11 (m, 2H) | |
| Example 5 | 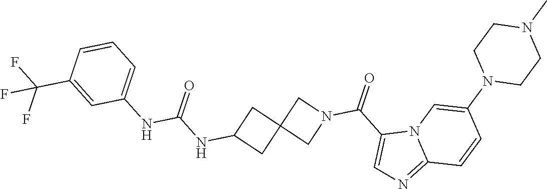 [SMILES: CN1CCN(c2ccc3ncc(C(=O)N4CC5(CC(NC(=O)Nc6cccc(C(F)(F)F)c6)C5)C4)n3c2)CC1] | 44 mg (24%) |
| | LCMS (ESI): Method 2, t_{R} = 2.90 min, m/z (M + 1) = 542.2 | |
| | ¹H NMR (400 MHz, CDCl₃) δ 9.09 (s, 1H), 7.81-7.77 (m, 2H), 7.61 (s, | |
| | 1H), 7.58-7.51 (m, 2H), 7.37-7.27 (m, 2H), 7.25-7.23 (m, 1H), 5.83- | |
| | 5.78 (m, 1H), 4.31-4.15 (m, 5H), 3.16 (t, J = 4.7 Hz, 4H), 2.74-2.66 (m, | |
| | 2H), 2.60 (t, J = 4.7 Hz, 4H), 2.37 (s, 3H), 2.14-2.06 (m, 2H) | |
| Example 6 | 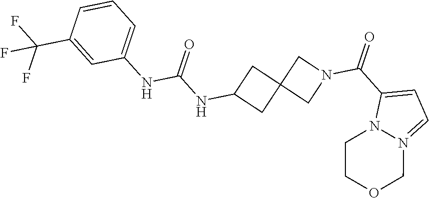 | 26 mg (23%) |
| | LCMS (ESI): Method 2, t_{R} = 3.38 min, m/z (M + 1) = 450.3 | |
| | ¹H NMR (400 MHz, DMSO-d6) δ 8.78 (s, 1H), 7.93 (s, 1H), 7.53-7.41 | |
| | (m, 2H), 7.35 (s, 1H), 7.22 (d, J = 7.6 Hz, 1H), 6.60 (d, J = 7.3 Hz, 1H), 4.76 | |
| | (s, 2H), 4.37 (d, J = 47.8 Hz, 2H), 4.22 (t, J = 5.2 Hz, 2H), 4.08-4.00 (m, | |
| | 2H), 3.99-3.93 (m, 3H), 2.56-2.52 (m, 2H), 2.17-2.09 (m, 2H) | |
| Example 7 | 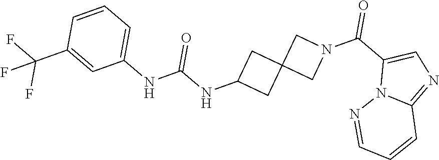 [SMILES: O=C(Nc1cccc(C(F)(F)F)c1)NC1CC2(C1)CN(C(=O)c1cnc3cccnn13)C2] | 18 mg (16%) |
| | LCMS (ESI): Method 2, t_{R} = 3.72 min, m/z (M + 1) = 445.4 | |
| | ¹H NMR (400 MHz, DMSO-d6) δ 8.75 (s, 1H), 8.67 (dd, J = 1.6, 4.4 Hz, | |
| | 1H), 8.23 (dd, J = 1.6, 9.2 Hz, 1H), 8.13 (s, 1H), 7.94-7.91 (m, 1H), 7.52- | |
| | 7.37 (m, 3H), 7.23-7.19 (m, 1H), 6.56 (d, J = 7.3 Hz, 1H), 4.38-3.97 | |
| | (m, 5H), 2.58-2.52 (m, 2H), 2.19-2.08 (m, 2H) | |
| Example 8 | 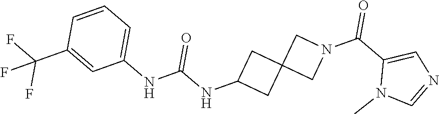 [SMILES: Cn1cncc1C(=O)N1CC2(CC(NC(=O)Nc3cccc(C(F)(F)F)c3)C2)C1] | 23 mg (22%) |
| | LCMS (ESI): Method 2, t_{R} = 3.15 min, m/z (M + 1) = 408.3 | |
| | ¹H NMR (400 MHz, DMSO-d6) δ 8.76 (s, 1H), 7.94-7.92 (m, 1H), 7.77 | |
| | (s, 1H), 7.52-7.41 (m, 2H), 7.34 (s, 1H), 7.22 (d, J = 7.3 Hz, 1H), 6.58 (d, | |
| | J = 7.8 Hz, 1H), 4.42-4.28 (m, 2H), 4.08-3.93 (m, 3H), 3.78 (s, 3H), 2.57- | |
| | 2.52 (m, 2H), 2.17-2.08 (m, 2H) | |
| Example 9 | 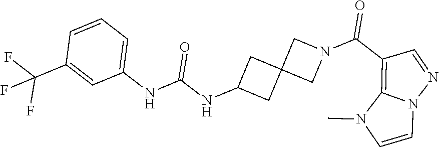 [SMILES: Cn1ccn2ncc(C(=O)N3CC4(CC(NC(=O)Nc5cccc(C(F)(F)F)c5)C4)C3)c12] | 47 mg (32%) |
| | LCMS (ESI): Method 2, t_{R} = 3.98 min, m/z (M + 1) = 447.2 | |
| | ¹H NMR (400 MHz, DMSO-d6) δ 8.80 (s, 1H), 7.94 (s, 1H), 7.77 (s, 1H), | |
| | 7.66-7.65 (m, 1H), 7.53-7.41 (m, 2H), 7.26-7.20 (m, 2H), 6.62 (d, | |
| | J = 7.6 Hz, 1H), 4.19-3.96 (m, 8H), 2.57-2.52 (m, 2H), 2.16-2.09 (m, | |
| | 2H) | |
| Example 10 | 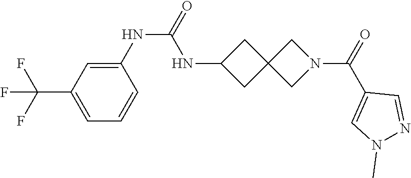 [SMILES: Cn1cc(C(=O)N2CC3(CC(NC(=O)Nc4cccc(C(F)(F)F)c4)C3)C2)cn1] | 48 mg (47%) |
| | LCMS (ESI): Method 3, t_{R} = 3.92 min, m/z (M + 1) = 408.2 | |
| | ¹H NMR (400 MHz, DMSO-d6) δ 8.79-8.77 (m, 1H), 8.10-8.07 (m, | |
| | 1H), 7.93 (s, 1H), 7.69 (s, 1H), 7.52-7.42 (m, 2H), 7.22 (d, J = 7.8 Hz, | |
| | 1H), 6.62-6.59 (m, 1H), 4.41-4.27 (m, 2H), 4.06-3.90 (m, 3H), 3.85 | |
| | (s, 3H), 2.16-2.09 (m, 2H) | |
| Example 30 | 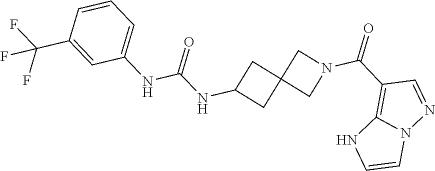 [SMILES: O=C(Nc1cccc(C(F)(F)F)c1)NC1CC2(C1)CN(C(=O)c1cnn3cc[nH]c13)C2] | 15 mg (14%) |
| | LCMS (ESI): Method 2, t_{R} = 3.89 min, m/z (M + 1) = 433.5 | |
| | ¹H NMR (400 MHz, DMSO-d6) δ 11.80 (s, 1H), 8.87 (s, 1H), 7.94 (s, | |
| | 1H), 7.75 (d, J = 0.8 Hz, 1H), 7.62 (d, J = 2.3 Hz, 1H), 7.51 (d, J = 9.2 Hz, | |
| | 1H), 7.47-7.41 (m, 1H), 7.24-7.19 (m, 2H), 6.70 (d, J = 7.6 Hz, 1H), 4.29- | |
| | 4.00 (m, 5H), 2.17-2.11 (m, 2H). | |
| Example 32 | 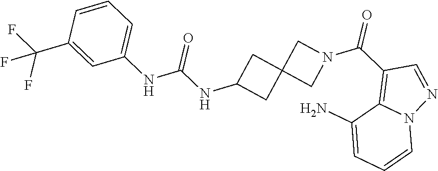 [SMILES: Nc1cccn2ncc(C(=O)N3CC4(CC(NC(=O)Nc5cccc(C(F)(F)F)c5)C4)C3)c12] | 20 mg (9%) |
| | LCMS (ESI): Method 2, t_{R} = 4.39 min, m/z (M + 1) = 459.2 | |
| | ¹H NMR (400 MHz, DMSO-d6) δ 8.76 (s, 1H), 8.17 (s, 1H), 7.98 (d, | |
| | J = 6.6 Hz, 1H), 7.94 (s, 1H), 7.51 (d, J = 8.6 Hz, 1H), 7.44 (t, J = 8.0 Hz, | |
| | 1H), 7.22 (d, J = 7.3 Hz, 1H), 6.96 (s, 2H), 6.82 (t, J = 7.2 Hz, 1H), 6.58 (d, | |
| | J = 7.8 Hz, 1H), 6.40 (d, J = 7.6 Hz, 1H), 4.56-4.44 (m, 2H), 4.13-4.02 | |
| | (m, 3H), 2.59-2.52 (m, 2H), 2.19-2.11 (m, 2H). | |
| Example 40 | 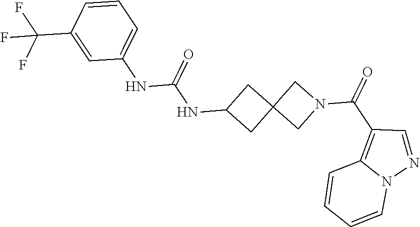 [SMILES: O=C(Nc1cccc(C(F)(F)F)c1)NC1CC2(C1)CN(C(=O)c1cnn3ccccc13)C2] | 56 mg (51%) |
| | LCMS (ESI): Method 2, t_{R} = 4.31 min, m/z (M + 1) = 444.3 | |
| | ¹H NMR (400 MHz, DMSO-d6) δ 8.80 (d, J = 7.1 Hz, 1H), 8.74 (s, 1H), | |
| | 8.29 (s, 1H), 8.20 (d, J = 8.8 Hz, 1H), 7.94 (s, 1H), 7.53-7.42 (m, 3H), | |
| | 7.22 (d, J = 7.6 Hz, 1H), 7.11-7.07 (m, 1H), 6.57 (d, J = 7.6 Hz, 1H), 4.54- | |
| | 4.41 (m, 2H), 4.13-4.00 (m, 3H), 2.59-2.52 (m, 2H), 2.19-2.12 (m, | |
| | 2H). | |
| Example 44 | 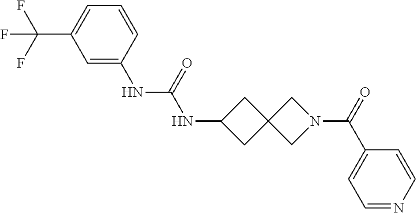 [SMILES: O=C(Nc1cccc(C(F)(F)F)c1)NC1CC2(C1)CN(C(=O)c1ccncc1)C2] | 20 mg (20%) |
| | LCMS (ESI): Method 3, t_{R} = 3.96 min, m/z (M + 1) = 405.5 | |
| | ¹H NMR (400 MHz, DMSO-d6) δ 8.74-8.67 (m, 3H), 7.92 (s, 1H), 7.57- | |
| | 7.53 (m, 2H), 7.49 (d, J = 8.6 Hz, 1H), 7.43 (dd, J = 7.8, 7.8 Hz, 1H), 7.22 | |
| | (d, J = 7.3 Hz, 1H), 6.56 (d, J = 7.3 Hz, 1H), 4.38 (s, 1H), 4.26 (s, 1H), 4.13 | |
| | (s, 1H), 4.05-3.96 (m, 2H), 2.56-2.52 (m, 2H), 2.16-2.07 (m, 2H). | |
| Example 46 | 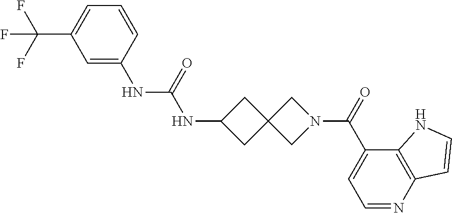 [SMILES: O=C(Nc1cccc(C(F)(F)F)c1)NC1CC2(C1)CN(C(=O)c1ccnc3cc[nH]c13)C2] | 37 mg (25%) |
| | LCMS (ESI): Method 2, t_{R} = 3.22 min, m/z (M + 1) = 444.2 | |
| | ¹H NMR-(400 MHz, DMSO-d6) δ 11.35-11.31 (m, 1H), 8.79 (d, J = 10.4 | |
| | Hz, 1H), 8.39 (dd, J = 4.8, 4.8 Hz, 1H), 7.93 (s, 1H), 7.63 (dd, J = 3.0, 3.0 | |
| | Hz, 1H), 7.49 (d, J = 8.3 Hz, 1H), 7.43 (dd, J = 7.8, 7.8 Hz, 1H), 7.23-7.18 | |
| | (m, 2H), 6.60 (d, J = 8.3 Hz, 2H), 4.36 (d, J = 49.6 Hz, 2H), 4.16 (d, J = 42.8 | |
| | Hz, 2H), 4.08-3.95 (m, 1H), 2.59-2.52 (m, 2H), 2.18-2.07 (m, 2H). | |
| Example 54 | 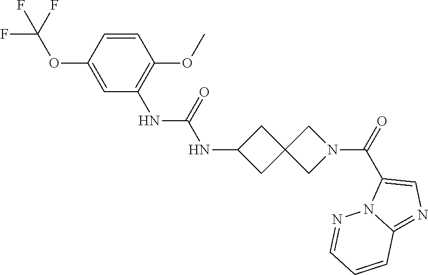 [SMILES: COc1ccc(OC(F)(F)F)cc1NC(=O)NC1CC2(C1)CN(C(=O)c1cnc3cccnn13)C2] | 30 mg (28%) |
| | LCMS (ESI): Method 3, t_{R} = 4.05 min, m/z (M + 1) = 491.2 | |
| | ¹H NMR (400 MHz, DMSO-d6) δ 8.67 (dd, J = 1.6, 4.4 Hz, 1H), 8.24 (dd, | |
| | J = 1.5, 9.3 Hz, 1H), 8.16-8.12 (m, 2H), 8.05 (s, 1H), 7.40-7.36 (m, 1H), | |
| | 7.26 (s, 1H), 7.02 (d, J = 8.8 Hz, 1H), 6.84 (dd, J = 2.7, 8.5 Hz, 1H), 4.36- | |
| | 3.97 (m, 5H), 3.86 (s, 3H), 2.60-2.52 (m, 2H), 2.05 (s, 2H). | |
| Example 55 | 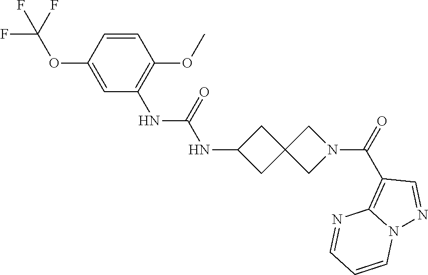 [SMILES: COc1ccc(OC(F)(F)F)cc1NC(=O)NC1CC2(C1)CN(C(=O)c1cnn3cccnc13)C2] | 62 mg (58%) |
| | LCMS (ESI): Method 3, t_{R} = 4.08 min, m/z (M + 1) = 491.2 | |
| | ¹H NMR (400 MHz, DMSO-d6) δ 9.22 (dd, J = 1.8, 7.1 Hz, 1H), 8.75 (d, | |
| | J = 2.5 Hz, 1H), 8.47 (s, 1H), 8.15 (d, J = 2.0 Hz, 1H), 8.05 (s, 1H), 7.29- | |
| | 7.18 (m, 2H), 7.02 (d, J = 8.8 Hz, 1H), 6.86-6.81 (m, 1H), 4.40 (s, 1H), | |
| | 4.30 (s, 1H), 4.12-3.92 (m, 3H), 3.86 (s, 3H), 2.58-2.52 (m, 2H), 2.08- | |
| | 1.99 (m, 2H). | |
| Example 57 | 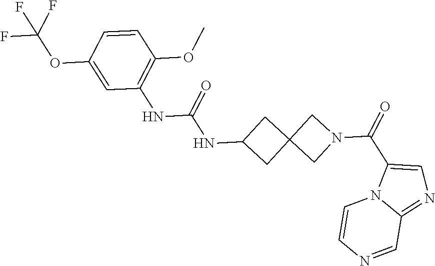 [SMILES: COc1ccc(OC(F)(F)F)cc1NC(=O)NC1CC2(C1)CN(C(=O)c1cnc3cnccn13)C2] | 21 mg (20%) |
| | LCMS (ESI): Method 3, t_{R} =4.17 min, m/z (M + 1) = 491.2 | |
| | ¹H NMR (400 MHz, DMSO-d6) δ 9.30 (dd, J = 1.5, 4.5 Hz, 1H), 9.24 (d, | |
| | J = 1.3 Hz, 1H), 8.24 (s, 1H), 8.16 (d, J = 2.3 Hz, 1H), 8.12 (d, J = 4.8 Hz, | |
| | 1H), 8.06 (s, 1H), 7.29 (d, J = 7.3 Hz, 1H), 7.03 (d, J = 9.1 Hz, 1H), 6.86- | |
| | 6.82 (m, 1H), 4.62 (s, 1H), 4.51 (s, 1H), 4.23-4.03 (m, 3H), 3.87 (s, 3H), | |
| | 2.62-2.55 (m, 2H), 2.12-2.04 (m, 2H). | |
| Example 62 | 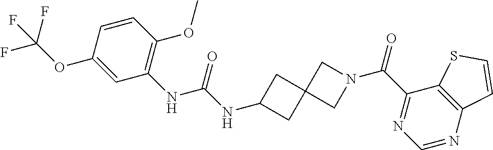 [SMILES: COc1ccc(OC(F)(F)F)cc1NC(=O)NC1CC2(C1)CN(C(=O)c1ncnc3ccsc13)C2] | 30 mg (20%) |
| | LCMS (ESI): Method 3, t_{R} = 4.85 min, m/z (M + 1) = 508.2 | |
| | ¹H NMR (400 MHz, DMSO-d6) δ 9.29 (d, J = 7.6 Hz, 1H), 8.59 (d, J = 5.6 | |
| | Hz, 1H), 8.16 (d, J = 3.0 Hz, 1H), 8.08 (d, J = 6.1 Hz, 1H), 7.68 (dd, J = 2.0, | |
| | 5.6 Hz, 1H), 7.29 (dd, J = 2.8, 7.3 Hz, 1H), 7.03 (d, J = 9.1 Hz, 1H), 6.84 | |
| | (dd, J = 2.1, 8.7 Hz, 1H), 4.86 (s, 1H), 4.77 (s, 1H), 4.25 (s, 1H), 4.16 (s, | |
| | 1H), 4.07-3.98 (m, 1H), 3.87 (s, 3H), 2.64-2.56 (m, 2H), 2.12-2.05 (m, | |
| | 2H). | |
| Example 69 | 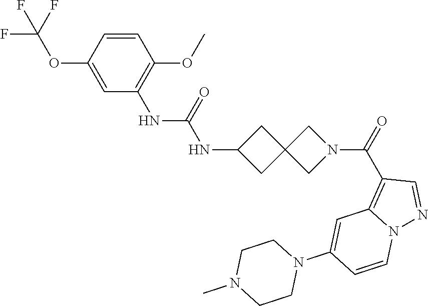 [SMILES: COc1ccc(OC(F)(F)F)cc1NC(=O)NC1CC2(C1)CN(C(=O)c1cnn3ccc(N4CCN(C)CC4)cc13)C2] | 39 mg (23%) |
| | LCMS (ESI): Method 3, t_{R} = 4.48 min, m/z (M + 1) = 588.5 | |
| | ¹H NMR (400 MHz, DMSO-d6) δ 8.52 (d, J = 7.7 Hz, 1H), 8.17 (d, J = 2.6 | |
| | Hz, 1H), 8.07 (d, J = 2.1 Hz, 2H), 7.34 (d, J = 2.6 Hz, 1H), 7.29 (d, J = 7.4 | |
| | Hz, 1H), 7.03 (d, J = 9.0 Hz, 1H), 6.96 (dd, J = 2.8, 7.8 Hz, 1H), 6.85 (dd, | |
| | J = 2.6, 8.7 Hz, 1H), 4.49-3.97 (m, 5H), 3.87 (s, 3H), 2.58-2.53 (m, 2H), | |
| | 2.25 (s, 3H), 2.09-2.02 (m, 2H). | |
| Example 70 | 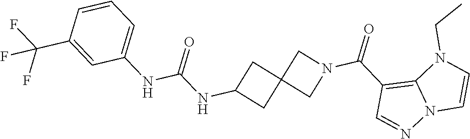 [SMILES: CCn1ccn2ncc(C(=O)N3CC4(CC(NC(=O)Nc5cccc(C(F)(F)F)c5)C4)C3)c12] | 9 mg (7%) |
| | LCMS (ESI): Method 3, t_{R} = 4.15 min, m/z (M + 1) = 461.45 | |
| | ¹H NMR (400 MHz, DMSO-d6) δ 8.77 (s, 1H), 7.95-7.93 (m, 1H), 7.78 | |
| | (d, J = 1.1 Hz, 1H), 7.67 (d, J = 2.3 Hz, 1H), 7.51 (d, J = 8.3 Hz, 1H), 7.44 (t, | |
| | J = 7.9 Hz, 1H), 7.35-7.33 (m, 1H), 7.22 (d, J = 7.5 Hz, 1H), 6.59 (d, J = 7.8 | |
| | Hz, 1H), 4.50-3.99 (m, 7H), 2.16-2.10 (m, 2H), 1.27 (t, J = 7.2 Hz, 3H). | |
| Example 84 | 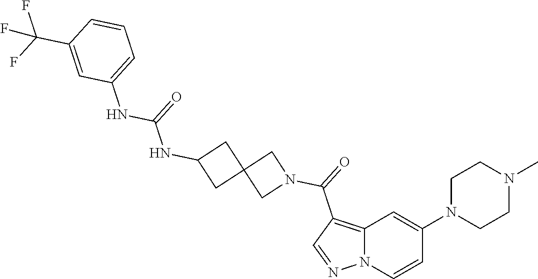 [SMILES: CN1CCN(c2ccn3ncc(C(=O)N4CC5(CC(NC(=O)Nc6cccc(C(F)(F)F)c6)C5)C4)c3c2)CC1] | 4 mg (2%) |
| | LCMS (ESI): Method 3, t_{R} = 3.27 min, m/z (M + 1) = 542.5 | |
| | ¹H NMR (400 MHz, DMSO-d6) δ 8.80 (s, 1H), 8.51 (d, J = 7.8 Hz, 1H), | |
| | 8.07 (s, 1H), 7.94 (s, 1H), 7.51 (d, J = 8.5 Hz, 1H), 7.44 (dd, J = 7.9, 7.9 | |
| | Hz, 1H), 7.33 (d, J = 2.6 Hz, 1H), 7.22 (d, J = 7.5 Hz, 1H), 6.95 (dd, J = 2.7, | |
| | 7.7 Hz, 1H), 6.63 (d, J = 7.5 Hz, 1H), 4.51-3.93 (m, 5H), 3.30-3.24 (m, | |
| | 4H), 2.57-2.52 (m, 2H), 2.45 (dd, J = 4.9, 4.9 Hz, 4H), 2.23 (s, 3H), 2.13 | |
| | (dd, J = 9.1, 11.9 Hz, 2H). | |
| Example 102 | 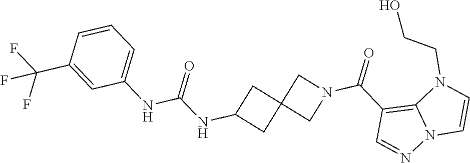 [SMILES: O=C(Nc1cccc(C(F)(F)F)c1)NC1CC2(C1)CN(C(=O)c1cnn3ccn(CCO)c13)C2] | 28 mg (8%) |
| | LCMS (ESI): Method 3, t_{R} = 3.79 min, m/z (M + 1) = 477.3 | |
| | ¹H NMR (400 MHz, DMSO-d6) δ 8.75 (s, 1H), 7.94 (s, 1H), 7.79 (s, | |
| | 1H), 7.65 (d, J = 2.1 Hz, 1H), 7.51 (d, J = 8.5 Hz, 1H), 7.45 (dd, J = 7.9, 7.9 | |
| | Hz, 1H), 7.27-7.21 (m, 2H), 6.57 (d, J = 7.5 Hz, 1H), 4.90 (dd, J = 5.1, | |
| | 5.1 Hz, 1H), 4.47 (dd, J = 5.3, 5.3 Hz, 2H), 4.42-4.00 (m, 5H), 3-65 (q, | |
| | J = 5.3 Hz, 2H), 2.57-2.53 (m, 2H), 2.18-2.08 (m, 2H). | |
| Example 113 | 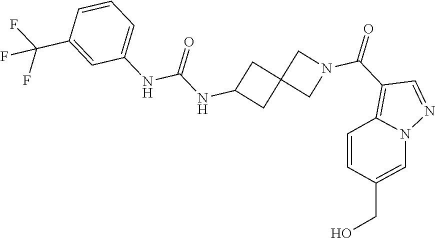 [SMILES: O=C(Nc1cccc(C(F)(F)F)c1)NC1CC2(C1)CN(C(=O)c1cnn3cc(CO)ccc13)C2] | 11 mg (10%) |
| | LCMS (ESI): Method 3, t_{R} = 4.06 min, m/z (M + 1) = 474.2 | |
| | ¹H NMR (400 MHz, DMSO-d6) δ 8.79 (s, 1H), 8.66 (s, 1H), 8.26 (s, 1H), | |
| | 8.16 (d, J = 9.0 Hz, 1H), 7.95 (s, 1H), 7.51 (d, J = 8.1 Hz, 1H), 7.45 (t, J = 8.9 | |
| | Hz, 2H), 7.23 (d, J = 7.6 Hz, 1H), 6.63 (d, J = 7.6 Hz, 1H), 5.43 (t, J = 5.7 Hz, | |
| | 1H), 4.58-4.52 (m, 3H), 4.46-4.42 (m, 1H), 4.17-3.94 (m, 3H), 2.60- | |
| | 2.55 (m, 2H), 2.19-2.08 (m, 2H). | |
| Example 116 | 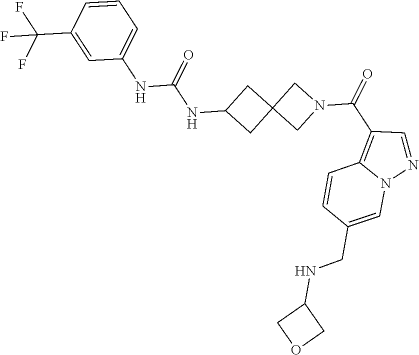 [SMILES: O=C(Nc1cccc(C(F)(F)F)c1)NC1CC2(C1)CN(C(=O)c1cnn3cc(CNC4COC4)ccc13)C2] | 9 mg (6%) |
| | LCMS (ESI): Method 3, t_{R} = 4.10 min, m/z (M + 1) = 529.3 | |
| | ¹H NMR (400 MHz, DMSO-d6) δ 8.81 (s, 1H), 8.68 (s, 1H), 8.25 (s, 1H), | |
| | 8.13 (d, J = 9.4 Hz, 1H), 7.95 (s, 1H), 7.52-7.43 (m, 3H), 7.22 (d, J = 7.6 | |
| | Hz, 1H), 6.68-6.61 (m, 1H), 4.57-4.27 (m, 6H), 4.16-3.84 (m, 4H), | |
| | 3.69 (d, J = 5.1 Hz, 2H), 3.12-3.03 (m, 1H), 2.19-2.09 (m, 3H). | |
| Example 135 | 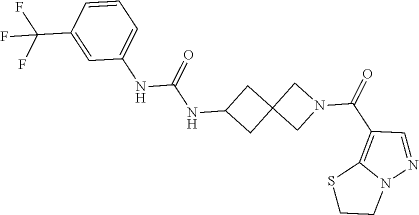 [SMILES: O=C(Nc1cccc(C(F)(F)F)c1)NC1CC2(C1)CN(C(=O)c1cnn3c1SCC3)C2] | 48 mg (42%) |
| | LCMS (ESI): Method 3, t_{R} = 4.01 min, m/z (M + 1) = 452.2 | |
| | ¹H NMR (400 MHz, DMSO-d6) δ 8.74 (s, 1H), 7.93 (s, 1H), 7.70 (s, | |
| | 1H), 7.52-7.41 (m, 2H), 7.22 (d, J = 7.6 Hz, 1H), 6.59-6.54 (m, 1H), | |
| | 4.42-4.29 (m, 4H), 4.08-3.83 (m, 5H), 2.16-2.08 (m, 2H). | |
| Example 136 | 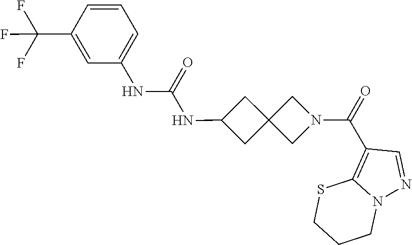 [SMILES: O=C(Nc1cccc(C(F)(F)F)c1)NC1CC2(C1)CN(C(=O)c1cnn3c1SCCC3)C2] | 60 mg (52%) |
| | LCMS (ESI): Method 3, t_{R} = 4.05 min, m/z (M + 1) = 466.2 | |
| | ¹H NMR (400 MHz, DMSO-d6) δ 8.74 (s, 1H), 7.93 (s, 1H), 7.68 (s, | |
| | 1H), 7.52-7.42 (m, 2H), 7.22 (d, J = 7.6 Hz, 1H), 6.56 (d, J = 7.6 Hz, 1H), | |
| | 4.40-4.15 (m, 4H), 4.08-3.88 (m, 3H), 3.03-2.99 (m, 2H), 2.27-2.20 | |
| | (m, 2H), 2.15-2.07 (m, 2H). | |
| Example 137 | 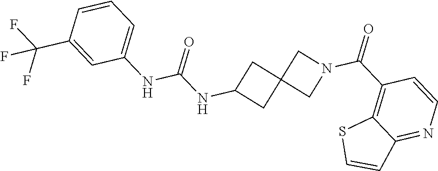 [SMILES: O=C(Nc1cccc(C(F)(F)F)c1)NC1CC2(C1)CN(C(=O)c1ccnc3ccsc13)C2] | 11 mg (9%) |
| | LCMS (ESI): Method 3, t_{R} = 4.29 min, m/z (M + 1) = 461.2 | |
| | ¹H NMR (400 MHz, DMSO-d6) δ 8.80-8.70 (m, 2H), 8.22 (d, J = 5.8 Hz, | |
| | 1H), 7.93 (s, 1H), 7.62-7.54 (m, 2H), 7.52-7.41 (m, 2H), 7.22 (d, J = 7.6 | |
| | Hz, 1H), 6.57-6.51 (m, 1H), 4.54 (d, J = 48.9 Hz, 2H), 4.25-3.98 (m, | |
| | 3H), 2.60-2.54 (m, 2H), 2.19-2.10 (m, 2H). | |
| Example 141 | 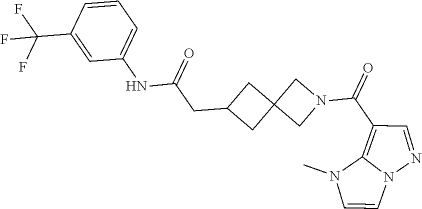 [SMILES: Cn1ccn2ncc(C(=O)N3CC4(CC(CC(=O)Nc5cccc(C(F)(F)F)c5)C4)C3)c12] | 56 mg (40%) |
| | LCMS (ESI): Method 3, t_{R} = 4.19 min, m/z (M + 1) = 446.2 | |
| | ¹H NMR (400 MHz, DMSO-d6) δ 10.19 (s, 1H), 8.09 (s, 1H), 7.77-7.73 | |
| | (m, 2H), 7.65 (d, J = 2.3 Hz, 1H), 7.53 (t, J = 8.0 Hz, 1H), 7.37 (d, J = 7.8 Hz, | |
| | 1H), 7.25-7.24 (m, 1H), 4.30-3.95 (m, 7H), 2.61-2.52 (m, 1H), 2.44 | |
| | (d, J = 7.7 Hz, 2H), 2.39-2.32 (m, 2H), 1.99-1.92 (m, 2H). | |
| Example 142 | 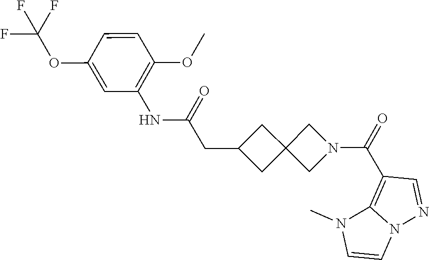 [SMILES: COc1ccc(OC(F)(F)F)cc1NC(=O)CC1CC2(C1)CN(C(=O)c1cnn3ccn(C)c13)C2] | 72 mg (48%) |
| | LCMS (ESI): Method 3, t_{R} = 4.42 min, m/z (M + 1) = 492.2 | |
| | ¹H NMR (400 MHz, DMSO-d6) δ 9.23 (s, 1H), 8.10 (d, J = 2.0 Hz, 1H), | |
| | 7.76 (d, J = 1.0 Hz, 1H), 7.65 (d, J = 2.3 Hz, 1H), 7.25-7.24 (m, 1H), 7.11 | |
| | (d, J = 9.1 Hz, 1H), 7.04 (dd, J = 2.6, 9.1 Hz, 1H), 4.23-3.87 (m, 10H), 2.56- | |
| | 2.52 (m, 3H), 2.38-2.30 (m, 2H), 1.98-1.92 (m, 2H). | |
| Example 147 | 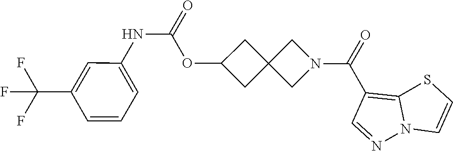 [SMILES: O=C(Nc1cccc(C(F)(F)F)c1)OC1CC2(C1)CN(C(=O)c1cnn3ccsc13)C2] | 14 mg (10%) |
| | LCMS (ESI): Method 6, t_{R} = 4.02 min, m/z (M + 1) = 451.20 | |
| | ¹H NMR (400 MHz, DMSO-d6) δ ppm 10.05 (1 H, s), 8.32 (1 H, d, | |
| | J = 4.17 Hz), 8.06 (1 H, s), 7.89 (1 H, s), 7.67 (1 H, br d, J = 8.33 Hz), 7.52 | |
| | (2 H, m), 7.34 (1 H, d, J = 7.67 Hz), 4.91 (1 H, br t, J = 7.23 Hz), 4.46 (2 | |
| | H, m), 4.08 (2 H, m), 2.70 (2 H, m), 2.33 (2 H, m). | |
| Example 148 | 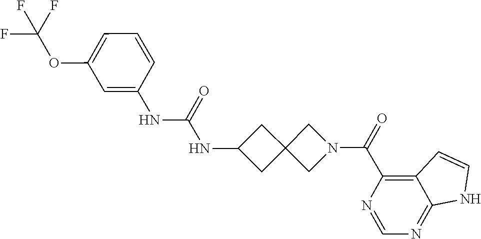 [SMILES: O=C(Nc1cccc(OC(F)(F)F)c1)NC1CC2(C1)CN(C(=O)c1ncnc3[nH]ccc13)C2] | 23 mg (26%) |
| | LCMS (ESI): Method 5, t_{R} = 5.19 min, m/z (M + 1) = 445.20 | |
| | ¹H NMR (400 MHz, DMSO-d6) δ ppm 12.32 (1 H, br s), 8.81 (1 H, d, | |
| | J = 7.23 Hz), 8.76 (1 H, d, J = 4.17 Hz), 7.94 (1 H, m), 7.69 (1 H, d, J = 3.29 | |
| | Hz), 7.46 (2 H, m), 7.22 (1 H, d, J = 7.45 Hz), 6.90 (1 H, dd, J = 3.40, 1.86 | |
| | Hz), 6.58 (1 H, br d, J = 7.45 Hz), 4.68 (1 H, s), 4.58 (1 H, s), 4.19 (1 H, | |
| | s), 4.08 (1 H, s), 4.06 (1 H, m), 4.04 (1 H, dd, J = 15.57, 7.67 Hz), 2.55 (2 | |
| | H, m), 2.14 (2 H, m). | |
| Example 166 | 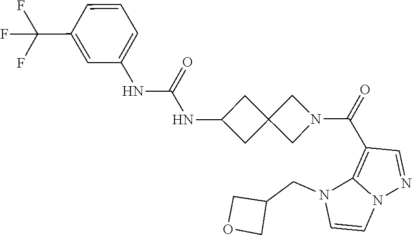 [SMILES: O=C(Nc1cccc(C(F)(F)F)c1)NC1CC2(C1)CN(C(=O)c1cnn3ccn(CC4COC4)c13)C2] | 22 mg (11%) |
| | LCMS (ESI): Method 2, t_{R} = 3.93 min, m/z (M + 1) = 503.5 | |
| | ¹H NMR (400 MHz, DMSO-d6) δ 8.75 (s, 1H), 7.94 (s, 1H), 7.79 (s, 1H), | |
| | 7.70 (d, J = 2.2 Hz, 1H), 7.54-7.38 (m, 3H), 7.23 (d, J = 7.3 Hz, 1H), 6.57 | |
| | (d, J = 7.6 Hz, 1H), 4.74 (d, J = 7.0 Hz, 2H), 4.57 (dd, J = 6.2, 7.9 Hz, 2H), | |
| | 4.40 (t, J = 6.1 Hz, 2H), 4.5-3.81 (m, 5H), 3.47-3.31 (m, 1H), 2.58-2.46 (m, | |
| | 2H), 2.19-2.08 (m, 2H). | |
| Example 167 | 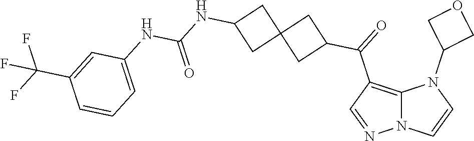 [SMILES: O=C(Nc1cccc(C(F)(F)F)c1)NC1CC2(C1)CC(C(=O)c1cnn3ccn(C4COC4)c13)C2] | 82 mg (52%) |
| | LCMS (ESI): Method 2, t_{R} = 3.94 min, m/z (M + 1) = 489.5 | |
| | ¹H NMR (400 MHz, DMSO-d6) δ 8.75 (s, 1H), 7.94 (s, 1H), 7.91 (s, 1H), | |
| | 7.86 (d, J = 2.4 Hz, 1H), 7.81 (s, 1H), 7.51 (d, J = 8.5 Hz, 1H), 7.44 (t, J = 7.9 | |
| | Hz, 1H), 7.23 (d, J = 7.6 Hz, 1H), 6.65 (p, J = 7.0 Hz, 1H), 6.57 (d, J = 7.5 | |
| | Hz, 1H), 4.91 (t, J = 7.4 Hz, 2H), 4.82 (t, J = 6.7 Hz, 2H), 4.52-3.87 (m, 5H), | |
| | 2.59-2.43 (m, 2H), 2.18-2.08 (m, 2H). | |
| Example 183 | 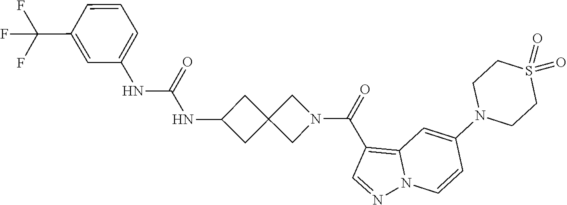 [SMILES: O=C(Nc1cccc(C(F)(F)F)c1)NC1CC2(C1)CN(C(=O)c1cnn3ccc(N4CCS(=O)(=O)CC4)cc13)C2] | 57 mg (30%) |
| | LCMS (ESI): Method 8, t_{R} = 2.66 min, m/z (M + 1) = 577.17 | |
| | ¹H NMR (400 MHz, DMSO-d6) δ 8.76 (s, 1H), 8.59 (d, J = 7.8 Hz, 1H), | |
| | 8.11 (s, 1H), 7.94 (d, J = 2.1 Hz, 1H), 7.50 (d, J = 8.3 Hz, 1H), 7.45 (d, J = | |
| | 7.8 Hz, 1H), 7.42 (d, J = 2.6 Hz, 1H), 7.22 (d, J = 7.5 Hz, 1H), 7.02 (dd, | |
| | J = 7.8, 2.9 Hz, 1H), 6.58 (d, J = 7.6 Hz, 1H), 4.35 (s, 3H), 4.05 (h, J = | |
| | 8.0 Hz, 2H), 3.91 (d, J = 5.5 Hz, 4H), 3.27-3.13 (m, 4H), 2.57-2.52 | |
| | (m, 2H), 2.14 (dd, J = 12.0, 8.7 Hz, 2H). | |
| Example 184 | 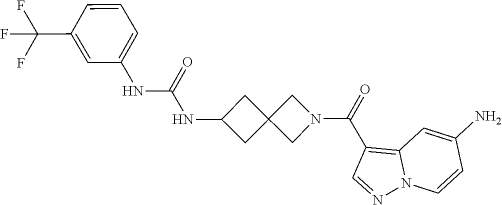 [SMILES: Nc1ccn2ncc(C(=O)N3CC4(CC(NC(=O)Nc5cccc(C(F)(F)F)c5)C4)C3)c2c1] | 27 mg (17%) |
| | LCMS (ESI): Method 8, t_{R} = 2.55 min, m/z (M + 1) = 459.16 | |
| | ¹H NMR (400 MHz, DMSO-d6) δ 8.74 (s, 1H), 8.34 (d, J = 7.4 Hz, 1H), | |
| | 7.94 (s, 2H), 7.53-7.48 (m, 1H), 7.44 (t, J = 7.9 Hz, 1H), 7.24-7.20 (m, | |
| | 1H), 7.11 (d, J = 2.5 Hz, 1H), 6.57 (d, J = 7.6 Hz, 1H), 6.45 (dd, J = 7.4, | |
| | 2.5 Hz, 1H), 6.10 (s, 2H), 4.19 (s, 4H), 4.04 (q, J = 8.0 Hz, 2H), 2.55 (s, | |
| | 2H), 2.17-2.08 (m, 2H). | |
| Example 185 | 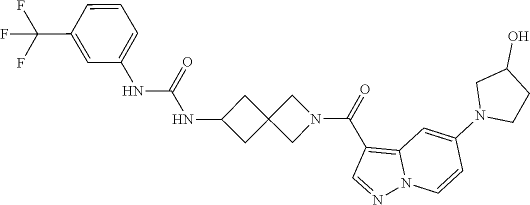 [SMILES: O=C(Nc1cccc(C(F)(F)F)c1)NC1CC2(C1)CN(C(=O)c1cnn3ccc(N4CCC(O)C4)cc13)C2] | 35 mg (19%) |
| | LCMS (ESI): Method 8, t_{R} = 2.56 min, m/z (M + 1) = 529.24 | |
| | ¹H NMR (400 MHz, DMSO-d6) δ 8.76 (s, 1H), 8.47 (d, J = 7.6 Hz, 1H), | |
| | 8.00 (s, 1H), 7.94 (d, J = 2.3 Hz, 1H), 7.50 (d, J = 8.3 Hz, 1H), 7.44 (t, | |
| | J = 7.9 Hz, 1H), 7.22 (d, J = 7.6 Hz, 1H), 6.92 (d, J = 2.6 Hz, 1H), 6.58 (dd, | |
| | J = 7.7, 2.6 Hz, 2H), 5.05 (d, J = 3.6 Hz, 1H), 4.43 (s, 1H), 4.05 (q, J = | |
| | 7.9 Hz, 2H), 3.49 (dd, J = 10.6, 4.7 Hz, 1H), 3.45-3.36 (m, 2H), 3.18 (d, | |
| | J = 10.5 Hz, 1H), 2.56-2.52 (m, 2H), 2.18-2.11 (m, 2H), 2.11-2.02 | |
| | (m, 2H), 1.95 (s, 2H). | |
| Example 186 | 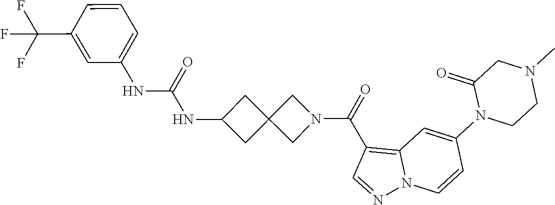 [SMILES: CN1CCN(c2ccn3ncc(C(=O)N4CC5(CC(NC(=O)Nc6cccc(C(F)(F)F)c6)C5)C4)c3c2)C(=O)C1] | 11 mg (6%) |
| | LCMS (ESI): Method 9, t_{R} = 2.31 min, m/z (M + 1) = 556.51 | |
| | ¹H NMR (400 MHz, DMSO-d6) δ 8.83-8.73 (m, 2H), 8.29 (s, 1H), 8.11- | |
| | 8.06 (m, 1H), 7.94 (d, J = 2.2 Hz, 1H), 7.54-7.48 (m, 1H), 7.44 (t, J = | |
| | 7.9 Hz, 1H), 7.22 (dd, J = 7.5, 2.3 Hz, 2H), 6.60 (d, J = 7.6 Hz, 1H), 4.53 | |
| | (s, 1H), 4.41 (s, 1H), 4.04 (dt, J = 16.0, 8.0 Hz, 3H), 3.79-3.72 (m, 2H), | |
| | 3.18 (s, 2H), 2.77 (t, J = 5.4 Hz, 2H), 2.56 (d, J = 9.9 Hz, 2H), 2.30 (s, | |
| | 3H), 2.20-2.10 (m, 2H). | |
| Example 194 | 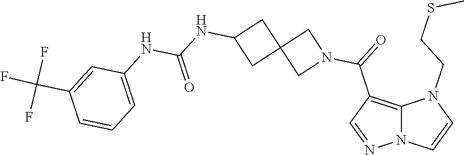 [SMILES: CSCCn1ccn2ncc(C(=O)N3CC4(CC(NC(=O)Nc5cccc(C(F)(F)F)c5)C4)C3)c12] | 1.3 mg (1%) |
| | LCMS (ESI): Method 2, t_{R} = 4.37 min, m/z (M + 1) = 507.5 | |
| | ¹H NMR (400 MHz, DMSO-d6) δ 8.79 (s, 1H), 7.94 (s, 1H), 7.78 (s, 1H), | |
| | 7.67 (d, J = 2.2 Hz, 1H), 7.51 (d, J = 10.2 Hz, 1H), 7.44 (t, J = 8.0 Hz, 1H), | |
| | 7.38-7.33 (m, 1H), 7.22 (d, J = 7.9 Hz, 1H), 6.61 (d, J = 7.5 Hz, 1H), 4.62 | |
| | (t, J = 7.3 Hz, 2H), 4.56-3.66 (m, 5H), 2.82 (t, J = 7.3 Hz, 2H), 2.57-2.46 | |
| | (m, 2H), 2.17-2.08 (m, 2H), 2.06 (s, 3H). | |
| Example 195 | 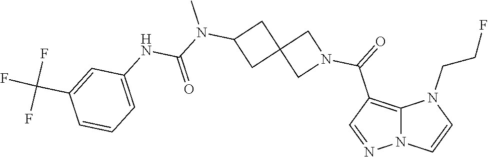 [SMILES: CN(C(=O)Nc1cccc(C(F)(F)F)c1)C1CC2(C1)CN(C(=O)c1cnn3ccn(CCF)c13)C2] | 22 mg (14%) |
| | LCMS (ESI): Method 2, t_{R} = 4.25 min, m/z (M + 1) = 493.2 | |
| | ¹H NMR (400 MHz, DMSO-d6) δ 8.59 (s, 1H), 7.91 (s, 1H), 7.80 (s, 1H), | |
| | 7.75 (d, J = 8.5 Hz, 1H), 7.71 (d, J = 2.2 Hz, 1H), 7.46 (t, J = 8.0 Hz, 1H), | |
| | 7.33 (s, 1H), 7.26 (d, J = 7.7 Hz, 1H), 4.75 (dt, J = 4.6, 9.0 Hz, 2H), 4.72 (dt, | |
| | J = 4.6, 70.3 Hz, 2H), 4.61-4.50 (m, 1H), 4.50-3.77 (m, 4H), 2.89 (s, 3H), | |
| | 2.46-2.31 (m, 4H). | |
| Example 196 | 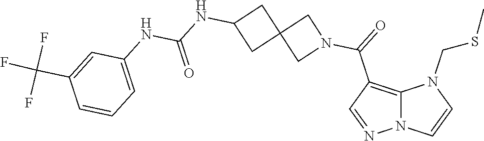 [SMILES: CSCn1ccn2ncc(C(=O)N3CC4(CC(NC(=O)Nc5cccc(C(F)(F)F)c5)C4)C3)c12] | 3.7 mg (5%) |
| | LCMS (ESI): Method 2, t_{R} = 4.30 min, m/z (M + 1) = 493.4 | |
| | ¹H NMR (400 MHz, DMSO-d6) δ 8.81 (s, 1H), 7.93 (s, 1H), 7.81 (s, 1H), | |
| | 7.73 (d, J = 2.3 Hz, 1H), 7.51 (d, J = 8.2 Hz, 1H), 7.45 (d, J = 7.8 Hz, 1H), | |
| | 7.43-7.39 (m, 1H), 7.22 (d, J = 7.8 Hz, 1H), 6.63 (d, J = 7.8 Hz, 1H), 5.97 | |
| | (s, 2H), 4.55-3.76 (m, 5H), 2.57-2.45 (m, 2H), 2.19-2.09 (m, 2H), 2.04 (s, | |
| | 3H). | |
| Example 201 | 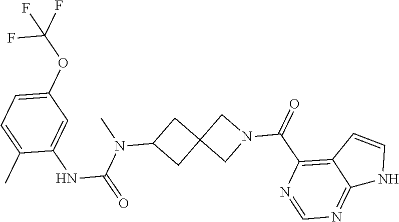 [SMILES: Cc1ccc(OC(F)(F)F)cc1NC(=O)N(C)C1CC2(C1)CN(C(=O)c1ncnc3[nH]ccc13)C2] | 12 mg (11%) |
| | LCMS (ESI): Method 5, t_{R} = 5.18 min, m/z (M + 1) = 489.28 | |
| | ¹H NMR (600 MHz, DMSO-d6) δ ppm 12.33 (1 H, br s), 8.80 (1 H, d, | |
| | J = 2.64 Hz), 7.81 (1 H, m), 7.69 (1 H, m), 7.34 (1 H, dd, J = 6.31, 1.61 | |
| | Hz), 7.28 (1 H, dd, J = 8.36, 1.61 Hz), 7.00 (1 H, br d, J = 8.36 Hz), 6.90 (1 | |
| | H, d, J = 3.23 Hz), 4.72 (1 H, s), 4.59 (1 H, s), 4.53 (1 H, m), 4.21 (1 H, | |
| | s), 4.09 (1 H, s), 2.89 (3 H, m), 2.41 (4 H, m), 2.18 (3 H, m). | |
| Example 203 | 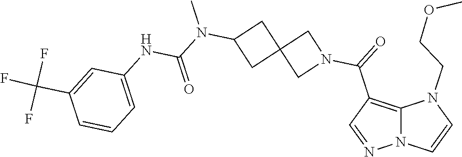 [SMILES: COCCn1ccn2ncc(C(=O)N3CC4(CC(N(C)C(=O)Nc5cccc(C(F)(F)F)c5)C4)C3)c12] | 6.2 mg (3.8%) |
| | LCMS (ESI): Method 2, t_{R} = 4.23 min, m/z (M + 1) = 505.5 | |
| | ¹H NMR (400 MHz, DMSO-d6) δ 8.60 (s, 1H), 7.91 (s, 1H), 7.78 (s, 1H), | |
| | 7.75 (d, J = 8.4 Hz, 1H), 7.65 (d, J = 2.2 Hz, 1H), 7.46 (t, J = 8.0 Hz, 1H), | |
| | 7.29-7.23 (m, 2H), 4.62 (t, J = 5.2 Hz, 2H), 4.61-4.50 (m, 1H), 4.50-3.71 | |
| | (m, 4H), 3.60 (t, J = 5.2 Hz, 2H), 3.22 (s, 3H), 2.89 (s, 3H), 2.46-2.30 (m, | |
| | 4H). | |
| Example 212 | 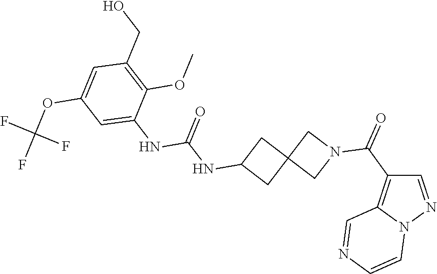 [SMILES: COc1c(CO)cc(OC(F)(F)F)cc1NC(=O)NC1CC2(C1)CN(C(=O)c1cnn3ccncc13)C2] | 60 mg (43%) |
| | LCMS (ESI): Method 9, t_{R} = 2.80 min, m/z (M + 1) = 521.30 | |
| | ¹H NMR (400 MHz, DMSO-d6) δ 9.56 (d, J = 1.5 Hz, 1H), 8.92 (dd, J = | |
| | 4.7, 1.4 Hz, 1H), 8.46 (s, 1H), 8.17 (s, 1H), 8.12 (t, J = 4.0 Hz, 2H), 7.31 | |
| | (d, J = 7.3 Hz, 1H), 6.93-6.87 (m, 1H), 5.32 (t, J = 5.8 Hz, 1H), 4.60 (s, | |
| | 1H), 4.56 (d, J = 5.7 Hz, 2H), 4.49 (s, 1H), 4.17 (s, 1H), 4.06 (s, 2H), 3.68 | |
| | (s, 3H), 2.60 (ddd, J = 10.0, 7.6, 2.9 Hz, 2H), 2.11 (dd, J = 11.8, 8.6 Hz, | |
| | 2H). | |
| Example 214 | 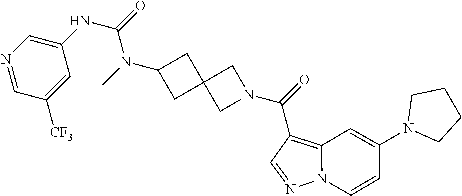 [SMILES: CN(C(=O)Nc1cncc(C(F)(F)F)c1)C1CC2(C1)CN(C(=O)c1cnn3ccc(N4CCCC4)cc13)C2] | 42 mg (25%) |
| | LCMS (ESI): Method 5, t_{R} = 6.46 min, m/z (M + 1) = 528.50 | |
| | ¹H NMR (400 MHz, DMSO-d6) δ ppm 8.94 (1 H, d, J = 2.19 Hz), 8.85 (1 | |
| | H, s), 8.52 (1 H, s), 8.47 (1 H, d, J = 7.67 Hz), 8.30 (1 H, m), 7.99 (1 H, s), | |
| | 6.94 (1 H, d, J = 2.63 Hz), 6.59 (1 H, dd, J = 7.67, 2.63 Hz), 4.57 (1 H, t, | |
| | J = 8.44 Hz), 4.21 (4 H, m), 3.34 (4 H, m), 2.92 (3 H, s), 2.41 (4 H, br dd, | |
| | J = 8.11, 3.51 Hz), 2.00 (4 H, m) | |
| Example 216 | 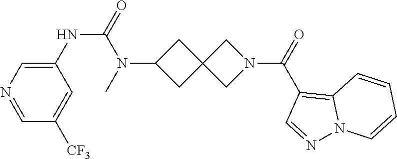 [SMILES: CN(C(=O)Nc1cncc(C(F)(F)F)c1)C1CC2(C1)CN(C(=O)c1cnn3ccccc13)C2] | 62 mg (22%) |
| | LCMS (ESI): Method 5, t_{R} = 5.22 min, m/z (M + 1) = 459.50 | |
| | ¹H NMR (400 MHz, DMSO-d6) δ ppm 8.94 (1 H, d, J = 2.19 Hz), 8.85 (1 | |
| | H, s), 8.80 (1 H, d, J = 7.02 Hz), 8.52 (1 H, m), 8.30 (1 H, d, J = 1.97 Hz), | |
| | 8.29 (1 H, br s), 8.20 (1 H, d, J = 8.77 Hz), 7.48 (1 H, ddd, J = 8.82, 6.85, | |
| | 0.99 Hz), 7.09 (1 H, td, J = 6.91, 1.32 Hz), 4.58 (1 H, quin, J = 8.50 Hz), | |
| | 4.43 (2 H, m), 4.14 (2 H, m), 2.92 (3 H, s), 2.43 (4 H, m). | |
| Example 225 | 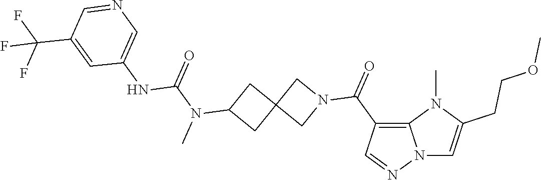 [SMILES: COCCc1cn2ncc(C(=O)N3CC4(CC(N(C)C(=O)Nc5cncc(C(F)(F)F)c5)C4)C3)c2n1C] | 20 mg (18%) |
| | LCMS (ESI): Method 13, t_{R} = 1.48 min, m/z (M + 1) = 520 | |
| | ¹H NMR (600 MHz, DMSO-d6) δ ppm 8.94 (1 H, d, J = 2.35 Hz), 8.84 (1 | |
| | H, s), 8.52 (1 H, s), 8.30 (1 H, t, J = 1.98 Hz), 7.72 (1 H, s), 7.48 (1 H, s), | |
| | 4.56 (1 H, t, J = 8.51 Hz), 3.96 (3 H, s), 4.20 (4 H, m), 3.59 (2 H, t, J = 6.53 | |
| | Hz), 3.28 (3 H, s), 2.92 (3 H, s), 2.88 (2 H, m), 2.40 (4 H, m). | |
| Example 238 | 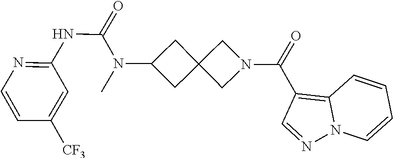 [SMILES: CN(C(=O)Nc1cc(C(F)(F)F)ccn1)C1CC2(C1)CN(C(=O)c1cnn3ccccc13)C2] | 40 mg (47%) |
| | LCMS (ESI): Method 5, t_{R} = 5.60 min, m/z (M + 1) = 459.40 | |
| | ¹H NMR (400 MHz, DMSO-d6) δ ppm 9.43 (1 H, s), 8.80 (1 H, d, J = 7.02 | |
| | Hz), 8.50 (1 H, d, J = 5.04 Hz), 8.28 (1 H, s), 8.20 (1 H, d, J = 8.77 Hz), 8.14 | |
| | (1 H, s), 7.48 (1 H, t, J = 7.85 Hz), 7.31 (1 H, d, J = 5.04 Hz), 7.09 (1 H, t, | |
| | J = 6.91 Hz), 4.56 (3 H, m), 4.07 (2 H, m), 2.91 (3 H, s), 2.41 (4 H, m). | |
| Example 239 | 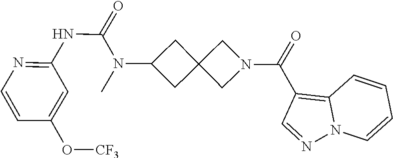 [SMILES: CN(C(=O)Nc1cc(OC(F)(F)F)ccn1)C1CC2(C1)CN(C(=O)c1cnn3ccccc13)C2] | 21 mg (25%) |
| | LCMS (ESI): Method 5, t_{R} = 5.71 min, m/z (M + 1) = 475.40 | |
| | ¹H NMR (400 MHz, DMSO-d6) δ ppm 9.28 (1 H, s), 8.76 (1 H, d, J = 7.02 | |
| | Hz), 8.30 (1 H, d, J = 5.48 Hz), 8.25 (1 H, s), 8.16 (1 H, d, J = 8.77 Hz), 7.80 | |
| | (1 H, s), 7.45 (1 H, dd, J = 8.33, 7.45 Hz), 7.06 (1 H, t, J = 6.91 Hz), 6.95 (1 | |
| | H, dd, J = 5.48, 1.10 Hz), 4.52 (3 H, m), 4.05 (2 H, m), 2.86 (3 H, s), 2.38 | |
| | (4 H, m). | |
| Example 247 | 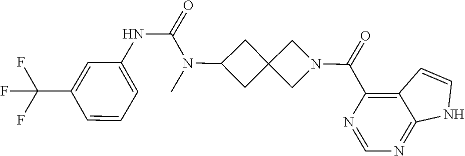 [SMILES: CN(C(=O)Nc1cccc(C(F)(F)F)c1)C1CC2(C1)CN(C(=O)c1ncnc3[nH]ccc13)C2] | 15 mg (28%) |
| | LCMS (ESI): Method 5, t_{R} = 5.41 min, m/z (M + 1) = 459.30 | |
| | ¹H NMR (400 MHz, DMSO-d6) δ ppm 12.33 (1 H, br s), 8.81 (1 H, d, | |
| | J = 5.92 Hz), 8.58 (1 H, d, J = 8.77 Hz), 7.90 (1 H, br s), 7.75 (1 H, br d, | |
| | J = 7.89 Hz), 7.69 (1 H, d, J = 3.29 Hz), 7.46 (1 H, t, J = 7.89 Hz), 7.26 (1 | |
| | H, d, J = 7.67 Hz), 6.91 (1 H, d, J = 3.29 Hz), 4.73 (1 H, s), 4.56 (2 H, m), | |
| | 4.22 (1 H, s), 4.09 (1 H, s), 2.89 (3 H, d, J = 3.73 Hz), 2.41 (4 H, m). | |
| Example 248 | 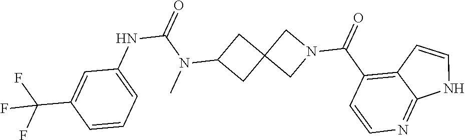 [SMILES: CN(C(=O)Nc1cccc(C(F)(F)F)c1)C1CC2(C1)CN(C(=O)c1ccnc3[nH]ccc13)C2] | 35 mg (47%) |
| | LCMS (ESI): Method 5, t_{R} = 5.48 min, m/z (M + 1) = 458.30 | |
| | ¹H NMR (400 MHz, DMSO-d6) δ ppm 11.87 (1 H, br s), 8.56 (1 H, br d, | |
| | J = 14.69 Hz), 8.28 (1 H, t, J = 3.95 Hz), 7.89 (1 H, br d, J = 9.65 Hz), 7.73 | |
| | (1 H, br t, J = 8.00 Hz), 7.59 (1 H, br s), 7.45 (1 H, br d, J = 5.26 Hz), 7.25 | |
| | (1 H, m), 7.16 (1 H, dd, J = 9.21, 4.82 Hz), 6.58 (1 H, br s), 4.53 (1 H, m), | |
| | 4.24 (1 H, s), 4.20 (1 H, s), 4.09 (1 H, s), 4.07 (1 H, s), 2.86 (3 H, d, | |
| | J = 16.00 Hz), 2.38 (4 H, m). | |
| Example 250 | 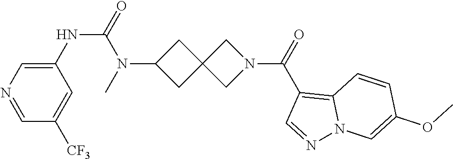 [SMILES: COc1ccc2c(C(=O)N3CC4(CC(N(C)C(=O)Nc5cncc(C(F)(F)F)c5)C4)C3)cnn2c1] | 27 mg (15%) |
| | LCMS (ESI): Method 5, t_{R} = 4.91 min, m/z (M + 1) = 489.25 | |
| | ¹H NMR (400 MHz, DMSO-d6) δ ppm 8.91 (1 H, d, J = 1.97 Hz), 8.81 (1 | |
| | H, s), 8.61 (1 H, d, J = 7.67 Hz), 8.48 (1 H, s), 8.27 (1 H, s), 8.14 (1 H, s), | |
| | 7.48 (1 H, d, J = 2.85 Hz), 6.72 (1 H, dd, J = 7.45, 2.85 Hz), 4.53 (2 H, m), | |
| | 4.38 (1 H, m), 4.12 (1 H, m), 3.98 (1 H, m), 3.83 (3 H, s), 2.89 (3 H, s), | |
| | 2.38 (4 H, m). | |
| Example 251 | 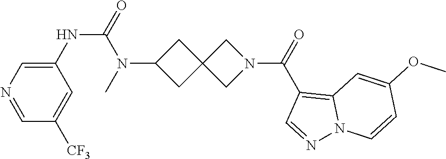 [SMILES: COc1ccn2ncc(C(=O)N3CC4(CC(N(C)C(=O)Nc5cncc(C(F)(F)F)c5)C4)C3)c2c1] | 35 mg (20%) |
| | LCMS (ESI): Method 5, t_{R} = 4.94 min, m/z (M + 1) = 489.27 | |
| | ¹H NMR (600 MHz, DMSO-d6) δ ppm 8.94 (1 H, d, J = 2.20 Hz), 8.85 (1 | |
| | H, s), 8.52 (1 H, d, J = 0.88 Hz), 8.51 (1 H, d, J = 2.05 Hz), 8.31 (1 H, t, | |
| | J = 1.91 Hz), 8.18 (1 H, br s), 8.09 (1 H, d, J = 9.54 Hz), 7.28 (1 H, dd, | |
| | J = 9.68, 2.20 Hz), 4.57 (2 H, m), 4.42 (1 H, br s), 4.13 (1 H, br s), 3.99 (1 | |
| | H, br s), 3.85 (3 H, s), 2.92 (3 H, s), 2.42 (4 H, m). | |
| Example 259 | 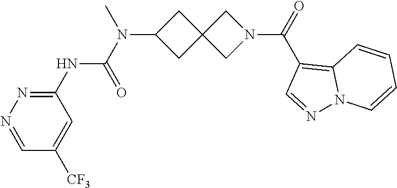 [SMILES: CN(C(=O)Nc1cc(C(F)(F)F)cnn1)C1CC2(C1)CN(C(=O)c1cnn3ccccc13)C2] | 78 mg (37%) |
| | LCMS (ESI): Method 5, t_{R} = 4.85 min, m/z (M + 1) = 459.99 | |
| | ¹H NMR (400 MHz, DMSO-d6) δ ppm 10.23 (1 H, br s), 9.26 (1 H, s), | |
| | 8.80 (1 H, d, J = 6.80 Hz), 8.33 (1 H, s), 8.29 (1 H, br s), 8.20 (1 H, d, | |
| | J = 8.99 Hz), 7.48 (1 H, t, J = 7.89 Hz), 7.09 (1 H, t, J = 6.91 Hz), 4.60 (2 H, | |
| | m), 4.43 (1 H, br s), 4.12 (1 H, m), 4.03 (1 H, m), 2.95 (3 H, s), 2.43 (4 | |
| | H, m). | |
| Example 260 | 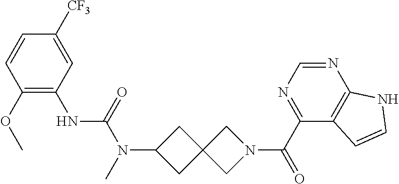 [SMILES: COc1ccc(C(F)(F)F)cc1NC(=O)N(C)C1CC2(C1)CN(C(=O)c1ncnc3[nH]ccc13)C2] | 10 mg (2.5%) |
| | LCMS (ESI): Method 5, t_{R} = 5.82 min, m/z (M + 1) = 488.97 | |
| | ¹H NMR (600 MHz, DMSO-d6) δ ppm 12.33 (1 H, br s), 8.81 (1 H, m), | |
| | 8.19 (1 H, m), 7.69 (1 H, dd, J = 3.37, 0.88 Hz), 7.53 (1 H, m), 7.34 (1 H, | |
| | br d, J = 8.51 Hz), 7.18 (1 H, m), 6.90 (1 H, m), 4.73 (1 H, s), 4.59 (1 H, | |
| | s), 4.48 (1 H, m), 4.23 (1 H, s), 4.09 (1 H, s), 3.92 (3 H, m), 2.88 (3 H, | |
| | m), 2.42 (4 H, m). | |
| Example 277 | 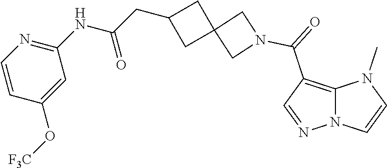 [SMILES: Cn1ccn2ncc(C(=O)N3CC4(CC(CC(=O)Nc5cc(OC(F)(F)F)ccn5)C4)C3)c12] | 60 mg (32%) |
| | LCMS (ESI): Method 5, t_{R} = 5.08 min, m/z (M + 1) = 463.24 | |
| | ¹H NMR (400 MHz, DMSO-d6) δ ppm 10.83 (1 H, s), 8.41 (1 H, d, J = 5.48 | |
| | Hz), 8.09 (1 H, s), 7.76 (1 H, s), 7.65 (1 H, d, J = 2.19 Hz), 7.25 (1 H, s), | |
| | 7.10 (1 H, br d, J = 5.70 Hz), 3.95 (3 H, s), 4.17 (4 H, m), 2.53 (3 H, m), | |
| | 2.35 (2 H, m), 1.93 (2 H, m). | |
| Example 278 | 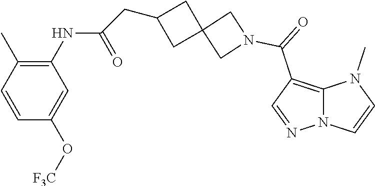 [SMILES: Cc1ccc(OC(F)(F)F)cc1NC(=O)CC1CC2(C1)CN(C(=O)c1cnn3ccn(C)c13)C2] | 75 mg (63%) |
| | LCMS (ESI): Method 5, t_{R} = 5.48 min, m/z (M + 1) = 476.27 | |
| | ¹H NMR (600 MHz, DMSO-d6) δ ppm 9.31 (1 H, s), 7.77 (1 H, s), 7.66 | |
| | (1 H, s), 7.54 (1 H, br s), 7.31 (1 H, d, J = 8.36 Hz), 7.25 (1 H, s), 7.05 (1 | |
| | H, br d, J = 8.22 Hz), 3.96 (3 H, s), 4.17 (4 H, m), 2.54 (1 H, m), 2.48 (2 | |
| | H, m), 2.36 (2 H, br t, J = 9.83 Hz), 2.22 (3 H, s), 1.97 (2 H, br t, J = 9.68 | |
| | Hz). | |
| Example 279 | 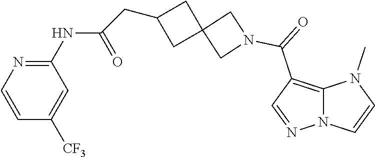 [SMILES: Cn1ccn2ncc(C(=O)N3CC4(CC(CC(=O)Nc5cc(C(F)(F)F)ccn5)C4)C3)c12] | 160 mg (56%) |
| | LCMS (ESI): Method 5, t_{R} = 4.88 min, m/z (M + 1) = 447.24 | |
| | ¹H NMR (400 MHz, DMSO-d6) δ ppm 10.93 (1 H, s), 8.58 (1 H, d, | |
| | J = 5.04 Hz), 8.41 (1 H, s), 7.76 (1 H, s), 7.66 (1 H, d, J = 2.19 Hz), 7.45 (1 | |
| | H, d, J = 5.04 Hz), 7.25 (1 H, s), 4.32 (4 H, m), 3.95 (3 H, s), 2.54 (3 H, | |
| | m), 2.35 (2 H, m), 1.94 (2 H, m). | |
| Intermediate 50 | 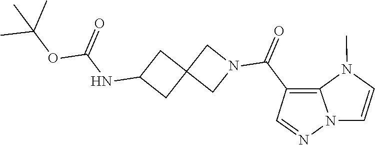 [SMILES: Cn1ccn2ncc(C(=O)N3CC4(CC(NC(=O)OC(C)(C)C)C4)C3)c12] | 2.3 g (quantitative) |
| | LCMS (ESI): Method 1, t_{R} = 1.37 min, m/z (M + 1) = 360.1 | |
| Intermediate 51 | 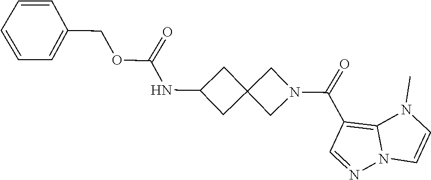 [SMILES: Cn1ccn2ncc(C(=O)N3CC4(CC(NC(=O)OCc5ccccc5)C4)C3)c12] | 660 mg (46%) |
| | LCMS (ESI): Method 10, t_{R} = 1.97 min, m/z (M + 1) = 393.90 | |
| Intermediate 52 | 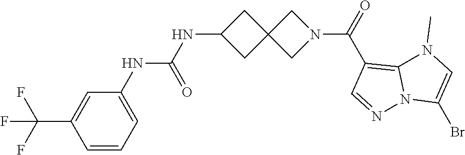 [SMILES: Cn1cc(Br)n2ncc(C(=O)N3CC4(CC(NC(=O)Nc5cccc(C(F)(F)F)c5)C4)C3)c12] | 62 mg (40%) |
| | LCMS (ESI): Method 7, t_{R} = 0.98 min, m/z (M + 1) = 526.99 | |
| Intermediate 53 | 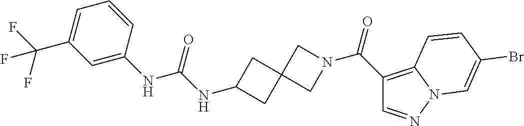 [SMILES: O=C(Nc1cccc(C(F)(F)F)c1)NC1CC2(C1)CN(C(=O)c1cnn3cc(Br)ccc13)C2] | 450 mg (43%) |
| | LCMS (ESI): Method 10, t_{R} = 2.16 min, m/z (M + 1) = 521.95 | |
| Intermediate 129 | 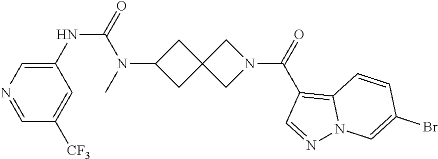 [SMILES: CN(C(=O)Nc1cncc(C(F)(F)F)c1)C1CC2(C1)CN(C(=O)c1cnn3cc(Br)ccc13)C2] | 112 mg (25%) |
| | LCMS (ESI): Method 7, t_{R} = 0.99 min, m/z (M + 1) = 538.97 | |
| Intermediate 130 | 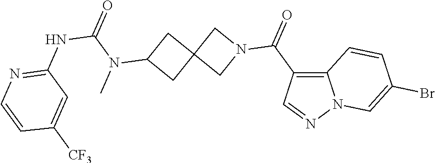 [SMILES: CN(C(=O)Nc1cc(C(F)(F)F)ccn1)C1CC2(C1)CN(C(=O)c1cnn3cc(Br)ccc13)C2] | 65 mg (40%) |
| | LCMS (ESI): Method 7, t_{R} = 1.08 min, m/z (M + 1) = 538.95 | |
| Intermediate 131 | 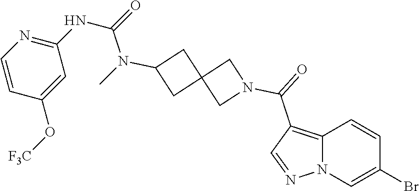 [SMILES: CN(C(=O)Nc1cc(OC(F)(F)F)ccn1)C1CC2(C1)CN(C(=O)c1cnn3cc(Br)ccc13)C2] | 36 mg (22%) |
| | LCMS (ESI): Method 7, t_{R} =1.08 min, m/z (M + 1) = 555.03/554.98 | |
| Intermediate 132 | 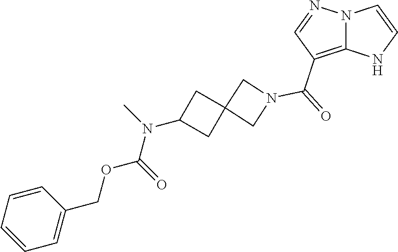 [SMILES: CN(C(=O)OCc1ccccc1)C1CC2(C1)CN(C(=O)c1cnn3cc[nH]c13)C2] | 960 mg (70%) |

LCMS (ESI): Method 7, t_{R} = 0.87 min, m/z (M + 1) = 394.18

### Example 11: 1-(2-(imidazo[1,2-a]pyrazin-3-ylmethyl)-2-azaspiro[3.3]heptan-6-yl)-3-(3-(trifluoromethyl)phenyl)urea

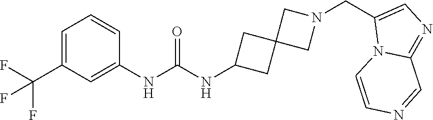 [SMILES: O=C(Nc1cccc(C(F)(F)F)c1)NC1CC2(C1)CN(Cc1cnc3cnccn13)C2]

### Example 11

A solution of Intermediate 7 (115 mg, 0.384 mmol), imidazo[1,2-a]pyrazine-3-carbaldehyde (62 mg, 0.423 mmol), STAB (122 mg, 0.576 mmol) and AcOH (2 drops) in DCM (3.00 mL) and MeOH (0.50 mL) was stirred at rt overnight. The reaction mixture was concentrated in vacuo and the residue was purified by achiral SFC (Method 19) to give the title compound (33 mg, 0.0766 mmol, 20% yield).

LCMS (ESI): Method 3, t_{R}=3.65 min, m/z (M+1)=431.27

¹H NMR (400 MHz, DMSO-d6) δ 9.03 (d, J=1.5 Hz, 1H), 8.72 (s, 1H), 8.45 (dd, J=1.5, 4.8 Hz, 1H), 7.93-7.90 (m, 2H), 7.72 (s, 1H), 7.49-7.40 (m, 2H), 7.20 (d, J=7.6 Hz, 1H), 6.55 (d, J=7.6 Hz, 1H), 4.02-3.91 (m, 1H), 3.89 (s, 2H), 3.21 (s, 2H), 3.09 (s, 2H), 2.41-2.34 (m, 2H), 2.00-1.93 (m, 2H).

The following Examples were prepared analogously to what described for Example 11 by starting from the suitable, commercially available aldehyde (see Table 16).

TABLE 16

| | | Product Amount |
|---|---|---|
| | Structure | (Yield) |
| No | Analytical Data | |
| Example 12 | 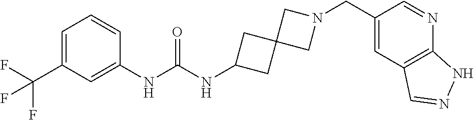 [SMILES: O=C(Nc1cccc(C(F)(F)F)c1)NC1CC2(C1)CN(Cc1cnc3[nH]ncc3c1)C2] | 7.3 mg (4.4%) |
| | LCMS (ESI): Method 2, t_{R} = 3.1 min, m/z (M + 1) = 431.2 | |
| | ¹H NMR (400 MHz, DMSO-d6) δ 13.55 (s, 1H), 8.76 (s, 1H), 8.40 (d, J = 2.0 Hz, | |
| | 1H), 8.09 (s, 1H), 8.06 (d, J = 1.8 Hz, 1H), 7.93-7.91 (m, 1H), 7.50-7.39 (m, 2H), | |
| | 7.22-7.18 (m, 1H), 6.59 (d, J = 7.6 Hz, 1H), 4.03-3.92 (m, 1H), 3.62 (s, 2H), 3.20 | |
| | (s, 2H), 3.08 (s, 2H), 2.43-2.35 (m, 2H), 2.01-1.94 (m, 2H) | |
| Example 13 | 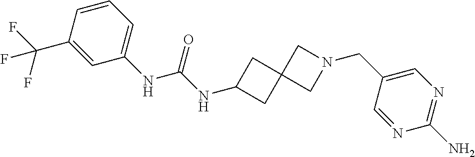 [SMILES: Nc1ncc(CN2CC3(CC(NC(=O)Nc4cccc(C(F)(F)F)c4)C3)C2)cn1] | 35 mg (23%) |
| | LCMS (ESI): Method 2, t_{R} = 2.98 min, m/z (M + 1) = 407 | |
| | ¹H NMR (400 MHz, DMSO-d6) δ 8.68 (s, 1H), 8.08-8.07 (m, 2H), 7.92 (s, 1H), | |
| | 7.49-7.39 (m, 2H), 7.20 (d, J = 7.6 Hz, 1H), 6.53-6.46 (m, 3H), 4.01-3.93 (m, 1H), | |
| | 3.27 (s, 2H), 3.12 (s, 2H), 3.01 (s, 2H), 2.40-2.33 (m, 2H), 1.99-1.92 (m, 2H). | |
| Example 14 | 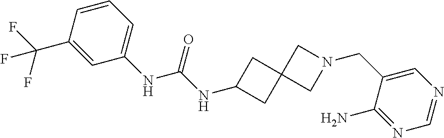 [SMILES: Nc1ncncc1CN1CC2(CC(NC(=O)Nc3cccc(C(F)(F)F)c3)C2)C1] | 79 mg (50%) |

LCMS (ESI): Method 3, t_{R} = 3.83 min, m/z (M + 1) = 407.2
¹H NMR (400 MHz, DMSO-d6) δ 8.68 (s, 1H), 7.92 (s, 1H), 7.83-7.82 (m, 1H),
7.63-7.62 (m, 1H), 7.50-7.40 (m, 2H), 7.21 (d, J = 7.3 Hz, 1H), 6.51-6.47 (m, 1H),
6.41-6.38 (m, 2H), 4.01-3.94 (m, 1H), 3.58 (s, 2H), 3.22 (s, 2H), 3.13-3.11 (m,
2H), 2.43-2.36 (m, 2H), 2.02-1.95 (m, 2H).

### Example 15: 1-(2-(1H-pyrazolo[3,4-b]pyridin-4-yl)-2-azaspiro[3.3]heptan-6-yl)-3-(3-(trifluoromethyl)phenyl)urea

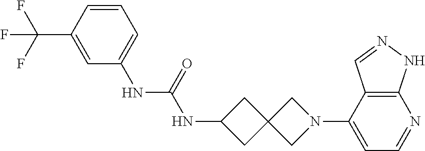 [SMILES: O=C(Nc1cccc(C(F)(F)F)c1)NC1CC2(C1)CN(c1ccnc3[nH]ncc13)C2]

### Example 15

### Step 1—1-(3-(trifluoromethyl)phenyl)-3-(2-(1-((2-(trimethylsilyl) ethoxy)methyl)-1H-pyrazolo[3,4-b]pyridin-4-yl)-2-azaspiro[3.3]heptan-6-yl)urea (Intermediate 8)

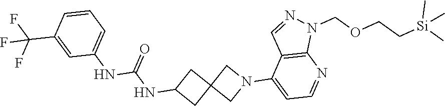 [SMILES: C[Si](C)(C)CCOCn1ncc2c(N3CC4(CC(NC(=O)Nc5cccc(C(F)(F)F)c5)C4)C3)ccnc21]

A suspension of Intermediate 5 (165 mg, 0.501 mmol), Intermediate 7 (150 mg, 0.501 mmol), RuPhos (47 mg, 0.100 mmol) and Cs₂CO₃ (490 mg, 1.50 mmol) in THF (5.00 mL) was degassed with N₂ for 10 min, then Pd₂(dba)₃ (46 mg, 0.0501 mmol) was added and the reaction mixture was stirred at 80° C. overnight. It was then cooled to rt, filtered through a pad of Celite®, washed with EtOAc (50 mL) and concentrated in vacuo. The residue was purified by FCC, (gradient of 20-100% EtOAc in cHex) to give the title compound (274 mg, 0.501 mmol, 100%).

LCMS (ESI): Method 4, t_{R}=1.77 min, m/z (M+1)=547.6

The following Examples and Intermediates were prepared using the procedure described for Intermediate 8, using the suitable commercially available bromo or chloro derivative or already described intermediates (see Table 17).

TABLE 17

| | | | Catalyst- |
|---|---|---|---|
| | | Product | Ligand |
| | | Amount | Base |
| | Structure | (Yield) | Solvent |
| No | Analytical Data | | |
| Example 138 | 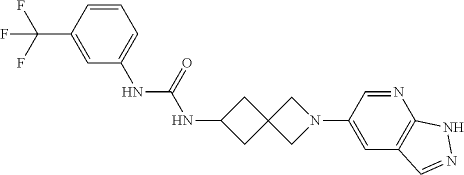 [SMILES: O=C(Nc1cccc(C(F)(F)F)c1)NC1CC2(C1)CN(c1cnc3[nH]ncc3c1)C2] | 20 mg (7%) | tBuBrettPhos Pd G3 Cs₂CO₃ MeTHF |
| | LCMS (ESI): Method 2, t_{R} = 4.11 min, m/z (M + 1) = 417.2 | | |
| | ¹H NMR (400 MHz, DMSO-d6) δ 13.26 (s, 1H), 8.76 (s, 1H), 7.96-7.89 (m, 3H), | | |
| | 7.51 (d, J = 8.6 Hz, 1H), 7.44 (t, J = 7.8 Hz, 1H), 7.23 (d, J = 6.8 Hz, 1H), 7.09 (d, | | |
| | J = 2.5 Hz, 1H), 6.59 (d, J = 7.6 Hz, 1H), 4.13-4.03 (m, 1H), 3.92 (s, 2H), 3.81 (s, | | |
| | 2H), 2.19-2.12 (m, 2H). | | |
| Example 149 | 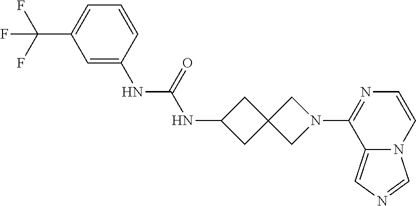 [SMILES: O=C(Nc1cccc(C(F)(F)F)c1)NC1CC2(C1)CN(c1nccn3cncc13)C2] | 19 mg (17%) | tBuBrettPhos Pd G3 Cs₂CO₃ MeTHF |
| | LCMS (ESI): Method 5, t_{R} = 4.11 min, m/z (M + 1) = 417.30 | | |
| | ¹H NMR (400 MHz, DMSO-d6) δ ppm 8.77 (1 H, s), 8.34 (1 H, s), 7.94 (1 H, s), | | |
| | 7.63 (1 H, d, J = 4.88 Hz), 7.56 (1 H, s), 7.51 (1 H, br d, J = 8.55 Hz), 7.44 (1 H, t, | | |
| | J = 7.89 Hz), 7.22 (1 H, d, J = 7.67 Hz), 7.01 (1 H, d, J = 4.82 Hz), 6.60 (1 H, d, J = 7.67 | | |
| | Hz), 4.34 (2 H, s), 4.23 (2 H, s), 4.08 (1 H, m), 2.57 (2 H, m), 2.18 (2 H, m) | | |
| Intermediate 54 | 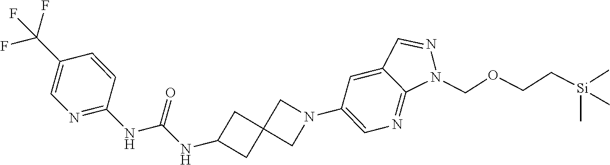 [SMILES: C[Si](C)(C)CCOCn1ncc2cc(N3CC4(CC(NC(=O)Nc5ccc(C(F)(F)F)cn5)C4)C3)cnc21] | 75 mg (38%) | tBuBrettPhos Pd G3 Cs₂CO₃ Dioxane |
| | LCMS (ESI): Method 4 min, t_{R} = 2.59 min, m/z (M + 1) = 548.09 | | |
| Intermediate 133 | 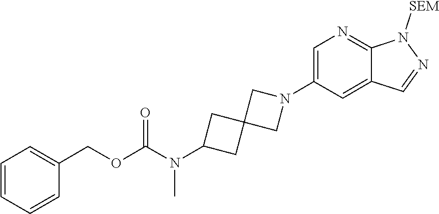 [SMILES: CN(C(=O)OCc1ccccc1)C1CC2(C1)CN(c1cnc3c(cnn3COCC[Si](C)(C)C)c1)C2] | 172 mg (67%) | tBuBrettPhos Pd G3 Cs₂CO₃ Dioxane |
| | LCMS (ESI): Method 13, t_{R} = 2.8 min, m/z (M + 1) = 508.25 | | |
| Intermediate 180 | 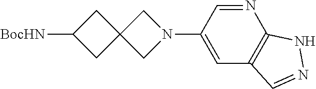 [SMILES: CC(C)(C)OC(=O)NC1CC2(C1)CN(c1cnc3[nH]ncc3c1)C2] | 172 mg (67%) | tBuBrettPhos Pd₂(dba)₃ potassium tert-butoxide CPME |

LCMS (ESI): Method 16, t_{R} = 1.5 min, m/z (M + 1) = 333.0

### Step 2—1-(2-(1/I-pyrazolo[3,4-b]pyridin-4-yl)-2-azaspiro[3.3]heptan-6-yl)-3-(3-(trifluoromethyl)phenyl)urea (Example 15)

A solution of Intermediate 8 (274 mg, 0.501 mmol) and HCl (4 M in 1,4-dioxane, 3.0 mL) in 1,4-dioxane (4.00 mL) was stirred at rt for 4 h, then concentrated in vacuo. The residue was purified by achiral SFC (Method 19) to give the title compound (67 mg, 0.161 mmol, 29% yield).

LCMS (ESI): Method 2, t_{R}=3.32 min, m/z (M+1)=417.2

¹H NMR (400 MHz, DMSO-d6) δ 13.11 (s, 1H), 8.82 (s, 1H), 8.00-7.94 (m, 3H), 7.54-7.42 (m, 2H), 7.24-7.20 (m, 1H), 6.69-6.64 (m, 1H), 5.86 (d, J=5.6 Hz, 1H), 4.34-4.03 (m, 5H), 2.63-2.56 (m, 2H), 2.24-2.17 (m, 2H).

### Example 163: 1-(2-(1H-pyrazolo[3,4-b]pyridin-5-yl)-2-azaspiro[3.3]heptan-6-yl)-3-(5-(trifluoro methyl)pyridin-2-yl)urea

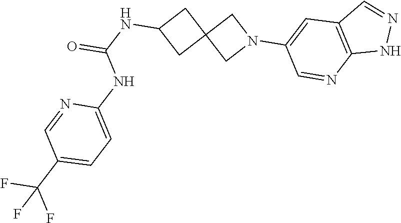 [SMILES: O=C(Nc1ccc(C(F)(F)F)cn1)NC1CC2(C1)CN(c1cnc3[nH]ncc3c1)C2]

### Example 163

Intermediate 54 (0.07 g, 0.13 mmol) was charged in a vial and diluted with TBAF 1M solution in THF (3.5 mL, 3.5 mmol). The solution was heated up to 70° C. for 8 h. Mixture was cooled down and organic mixture was diluted in EtOAc and washed with aq KHCO₃ 2% and then brine/water 1:4. Organic layer was evaporated to dryness and crude dissolved in DMF/CH₃CN 2:1 and purified by RF FCC. Fractions containing the products were combined and evaporated to dryness to give the title compound (0.025 g, 0.06 mmol, 47% yield).

LC-MS (ESI): Method 5, t_{R}=5.50 min, m/z (M+1)=418.30

¹H NMR (400 MHz, DMSO-d6) δ ppm 13.27 (1H, s), 9.45 (1H, s), 8.46 (1H, d, J=5.26 Hz), 7.92 (3H, m), 7.69 (1H, br d, J=7.23 Hz), 7.27 (1H, dd, J=5.26, 0.88 Hz), 7.09 (1H, d, J=2.41 Hz), 4.12 (1H, d, J=7.89 Hz), 3.93 (2H, s), 3.83 (2H, s), 2.57 (2H, m), 2.16 (2H, m)

The following Examples and Intermediates were prepared using the procedure described for Example 163 (see Table 18).

TABLE 18

| | | Product Amount |
|---|---|---|
| | Structure | (Yield) |
| No | Analytical Data | |
| Example 220 | 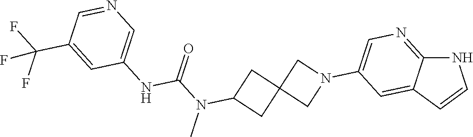 [SMILES: CN(C(=O)Nc1cncc(C(F)(F)F)c1)C1CC2(C1)CN(c1cnc3[nH]ccc3c1)C2] | 10 mg (15%) |
| | LC-MS (ESI): Method 5, t_{R} = 4.98 min, m/z (M + 1) = 461.40 | |
| | ¹H NMR (600 MHz, DMSO-d6) δ ppm 11.25 (1 H, br s), 8.95 (1 H, d, J = 2.35 Hz), | |
| | 8.87 (1 H, s), 8.52 (1 H, s), 8.32 (1 H, t, J = 2.05 Hz), 7.58 (1 H, d, J = 2.64 Hz), 7.33 | |
| | (1 H, t, J = 2.93 Hz), 6.97 (1 H, d, J = 2.49 Hz), 6.25 (1 H, dd, J = 3.23, 1.91 Hz), 4.61 | |
| | (1 H, t, J = 8.51 Hz), 3.90 (2 H, s), 3.77 (2 H, s), 2.94 (3 H, s), 2.41 (4 H, m) | |
| Intermediate 134 | 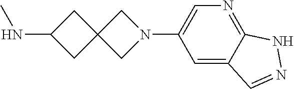 [SMILES: CNC1CC2(C1)CN(c1cnc3[nH]ncc3c1)C2] | 68 mg, (quantitative) |
| | LCMS (ESI): Method 7, t_{R} = 0.16 min, m/z (M + 1) = 244.27 | |

### Example 16: 1-(3-fluoro-5-(trifluoromethyl)phenyl)-3-(2-(pyrazolo[1,5-a]pyrazine-3-carbonyl)-2-azaspiro[3.3]heptan-6-yl)urea

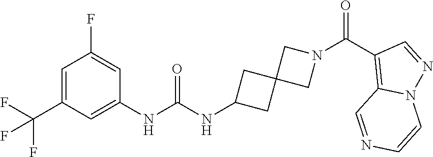 [SMILES: O=C(Nc1cc(F)cc(C(F)(F)F)c1)NC1CC2(C1)CN(C(=O)c1cnn3ccncc13)C2]

### Example 16

### Step 1—tert-butyl(2-(pyrazolo[1,5-a]pyrazine-3-carbonyl)-2-azaspiro[3.3]heptan-6-yl)carbamate (Intermediate 9)

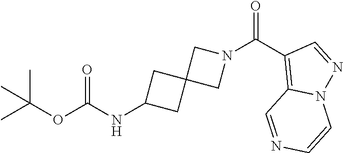 [SMILES: CC(C)(C)OC(=O)NC1CC2(C1)CN(C(=O)c1cnn3ccncc13)C2]

A solution of pyrazolo[1,5-a]pyrazine-3-carboxylic acid (384 mg, 2.36 mmol) and thionyl chloride (1.7 mL, 23.6 mmol) was stirred at reflux for 6 h. The reaction mixture was concentrated in vacuo and dissolved in DCM (2.00 mL). This solution was added dropwise to a solution of tert-butyl N-(2-azaspiro[3.3]heptan-6-yl)carbamate (500 mg, 2.36 mmol) and TEA (0.98 mL, 7.07 mmol) in DCM (2.00 mL) and the reaction was stirred at rt for 16 h. Water was added and the organic phase was separated. The organic phase was dried over a hydrophobic frit and concentrated in vacuo to give the title compound (750 mg, 2.10 mmol, 89% yield).

LCMS (ESI): Method 1, t_{R}=1.34 min, m/z (M+1)=358

¹H NMR (400 MHz, DMSO-d6) δ 9.55 (d, J=1.4 Hz, 1H), 8.91 (dd, J=1.4, 4.6 Hz, 2H), 8.44 (s, 1H), 8.11 (d, J=4.7 Hz, 1H), 7.16 (d, J=8.2 Hz, 1H), 4.50 (d, J=50.8 Hz, 2H), 4.04 (d, J=48.4 Hz, 2H), 3.91-3.79 (m, 1H), 2.48-2.43 (m, 2H), 2.15-2.07 (m, 2H), 1.38 (s, 9H).

Analogously, the following Examples and Intermediates were prepared starting from the suitable analogue and carboxylic acid (see Table 19).

TABLE 19

| | | Product Amount |
|---|---|---|
| | Structure | (Yield) |
| No | Analytical Data | |
| Example 73 | 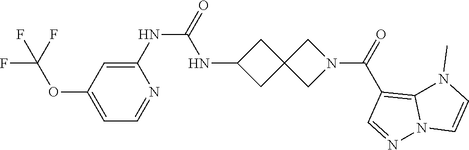 [SMILES: Cn1ccn2ncc(C(=O)N3CC4(CC(NC(=O)Nc5cc(OC(F)(F)F)ccn5)C4)C3)c12] | 21 mg (12%) |
| | LCMS (ESI): Method 2, t_{R} = 3.83 min, m/z (M + 1) = 464.3 | |
| | ¹H NMR (400 MHz, DMSO-d6) δ 9.34 (s, 1H), 8.30 (d, J = 5.9 Hz, 1H), 7.76 | |
| | (d, J = 1.1 Hz, 1H), 7.70 (d, J = 7.4 Hz, 1H), 7.66 (d, J = 2.4 Hz, 1H), 7.57 (s, 1H), | |
| | 7.25 (dd, J = 1.3, 2.1 Hz, 1H), 6.96-6.92 (m, 1H), 4.19-3.96 (m, 8H), 2.59-2.52 | |
| | (m, 2H), 2.16-2.09 (m, 2H). | |
| Example 77 | 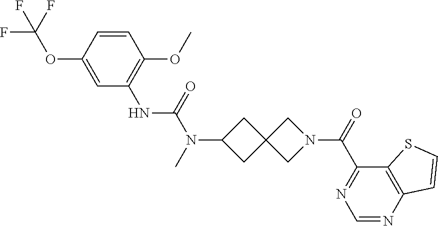 [SMILES: COc1ccc(OC(F)(F)F)cc1NC(=O)N(C)C1CC2(C1)CN(C(=O)c1ncnc3ccsc13)C2] | 7 mg (8%) |
| | LCMS (ESI): Method 2, t_{R} = 5.19 min, m/z (M + 1) = 522 | |
| | ¹H NMR (400 MHz, DMSO-d6) δ 9.29 (d, J = 7.3 Hz, 1H), 8.60 (d, J = 5.5 Hz, | |
| | 1H), 7.93 (dd, J = 3.3, 4.6 Hz, 1H), 7.69 (dd, J = 1.8, 5.6 Hz, 1H), 7.47 (s, 1H), | |
| | 7.08 (d, J = 8.5 Hz, 1H), 6.95 (dd, J = 2.6, 8.9 Hz, 1H), 4.91 (s, 1H), 4.76 (s, | |
| | 1H), 4.55-4.43 (m, 1H), 4.29 (s, 1H), 4.15 (s, 1H), 3.88 (d, J = 2.5 Hz, 3H), | |
| | 2.89 (s, 3H), 2.47-2.39 (m, 4H). | |
| Example 91 | 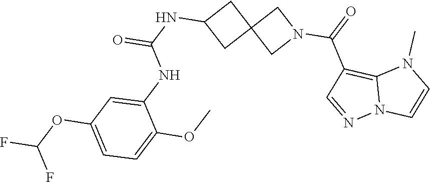 [SMILES: COc1ccc(OC(F)F)cc1NC(=O)NC1CC2(C1)CN(C(=O)c1cnn3ccn(C)c13)C2] | 39 mg (27%) |
| | LCMS (ESI): Method 2, t_{R} = 3.86 min, m/z (M + 1) = 475.32 | |
| | ¹H NMR (400 MHz, DMSO-d6) δ 8.01 (d, J = 2.9 Hz, 1H), 7.98 (s, 1H), 7.78 | |
| | (s, 1H), 7.67 (d, J = 2.1 Hz, 1H), 7.26 (d, J = 7.3 Hz, 2H), 7.22-6.83 (m, 2H), | |
| | 6.68 (dd, J = 2.9, 8.8 Hz, 1H), 4.18-3.97 (m, 8H), 3.84 (s, 3H), 2.08-2.01 (m, | |
| | 2H). | |
| Example 92 | 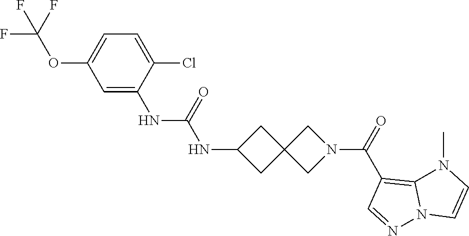 [SMILES: Cn1ccn2ncc(C(=O)N3CC4(CC(NC(=O)Nc5cc(OC(F)(F)F)ccc5Cl)C4)C3)c12] | 83 mg (59%) |
| | LCMS (ESI): Method 2, t_{R} = 4.47 min, m/z (M + 1) = 497.31 | |
| | ¹H NMR (400 MHz, DMSO-d6) δ 8.28 (d, J = 2.3 Hz, 1H), 8.16 (s, 1H), 7.77 | |
| | (s, 1H), 7.66 (d, J = 2.1 Hz, 1H), 7.54 (d, J = 8.9 Hz, 1H), 7.48 (d, J = 7.1 Hz, | |
| | 1H), 7.26 (s, 1H), 6.95 (dd, J = 2.8, 8.8 Hz, 1H), 4.19-3.95 (m, 8H), 2.60- | |
| | 2.54 (m, 2H), 2.12-2.05 (m, 2H). | |
| Example 103 | 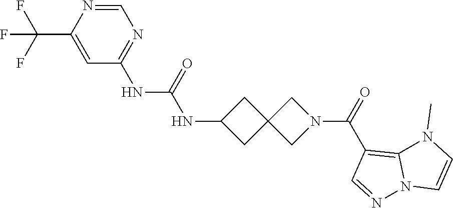 [SMILES: Cn1ccn2ncc(C(=O)N3CC4(CC(NC(=O)Nc5cc(C(F)(F)F)ncn5)C4)C3)c12] | 83 mg (52%) |
| | LCMS (ESI): Method 2, t_{R} = 3.46 min, m/z (M + 1) = 449.4 | |
| | ¹H NMR (400 MHz, DMSO-d6) δ 10.01 (br s, 1H), 8.93 (s, 1H), 8.08 | |
| | (s, 1H), 7.77 (s, 1H), 7.67 (d, J = 2.2 Hz, 1H), 7.61-7.52 (m, 1H), 7.26 | |
| | (s, 1H), 4.54-3.80 (m, 5H), 3.97 (s, 3H), 2.62-2.40 (m, 2H, partly | |
| | covered by the residual DMSO peak), 2.22-2.09 (m, 2H). | |
| Example 174 | 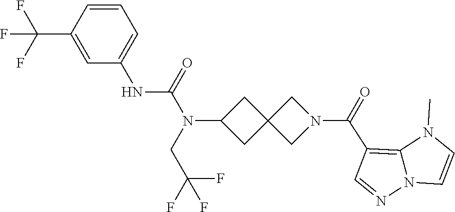 [SMILES: Cn1ccn2ncc(C(=O)N3CC4(CC(N(CC(F)(F)F)C(=O)Nc5cccc(C(F)(F)F)c5)C4)C3)c12] | 25 mg (18%) |
| | LCMS (ESI): Method 2, t_{R} = 4.65 min, m/z (M + 1) = 529.6 | |
| | ¹H NMR (400 MHz, DMSO-d6) δ 8.82 (s, 1H), 7.89 (s, 1H), 7.80-7.72 | |
| | (m, 2H), 7.66 (d, J = 2.2 Hz, 1H), 7.51 (t, J = 7.9 Hz, 1H), 7.34 (d, J = 7.6 | |
| | Hz, 1H), 7.28-7.24 (m, 1H), 4.58-3.76 (m, 7H), 3.96 (s, 3H), 2.62-2.43 | |
| | (m, 2H), 2.38-2.27 (m, 2H). | |
| Example 187 | 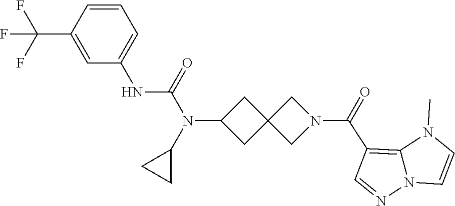 [SMILES: Cn1ccn2ncc(C(=O)N3CC4(CC(N(C(=O)Nc5cccc(C(F)(F)F)c5)C5CC5)C4)C3)c12] | 120 mg (21%) |
| | LCMS (ESI): Method 2, t_{R} = 4.44 min, m/z (M + 1) = 487.2 | |
| | ¹H NMR (400 MHz, DMSO-d6) δ 8.62 (s, 1H), 7.98 (s, 1H), 7.82-7.75 | |
| | (m, 2H), 7.66 (d, J = 2.2 Hz, 1H), 7.47 (t, J = 8.0 Hz, 1H), 7.29-7.23 (m, | |
| | 2H), 4.61-3.62 (m, 5H), 3.97 (s, 3H), 2.69-2.60 (m, 1H), 2.53-2.45 (m, | |
| | 4H), 0.96-0.88 (m, 2H), 0.67-0.60 (m, 2H). | |
| Example 191 | 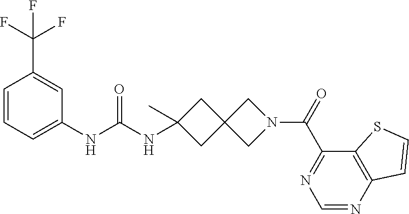 [SMILES: CC1(NC(=O)Nc2cccc(C(F)(F)F)c2)CC2(CN(C(=O)c3ncnc4ccsc34)C2)C1] | 23 mg (15%) |
| | LCMS (ESI): Method 2, t_{R} = 4.86 min, m/z (M + 1) = 476.3 | |
| | ¹H NMR (400 MHz, DMSO-d₆) δ 9.30 (d, J = 6.7 Hz, 1H), 8.69 (d, | |
| | J = 3.3 Hz, 1H), 8.60 (dd, J = 1.0, 4.9 Hz, 1H), 7.98 (s, 1H), 7.69 (dd, | |
| | J = 2.3, 5.6 Hz, 1H), 7.45 (d, J = 4.0 Hz, 2H), 7.23 (dd, J = 3.3, 3.3 Hz, | |
| | 1H), 6.48 (d, J = 2.5 Hz, 1H), 4.84 (d, J = 22.0 Hz, 2H), 4.22 (d, J = 20.6 | |
| | Hz, 2H), 2.62-2.56 (m, 2H), 2.55 (s, 1H), 2.34-2.27 (m, 2H), 1.37 (d, | |
| | J = 5.6 Hz, 3H). | |
| Example 274 | 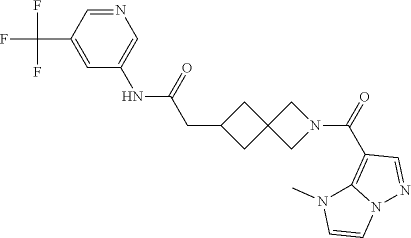 [SMILES: Cn1ccn2ncc(C(=O)N3CC4(CC(CC(=O)Nc5cncc(C(F)(F)F)c5)C4)C3)c12] | 57 mg (48%) |
| | LCMS (ESI): Method 2, t_{R} = 3.66 min, m/z (M + 1) = 447.4 | |
| | ¹H NMR (400 MHz, DMSO-d6) δ 10.48 (s, 1H), 8.91 (d, J = 2.0 Hz, 1H), | |
| | 8.63 (s, 1H), 8.50 (s, 1H), 7.76 (s, 1H), 7.66 (d, J = 2.2 Hz, 1H), 7.25 (s, | |
| | 1H), 4.52-3.79 (m, 4H), 3.96 (s, 3H), 2.62-2.45 (m, 1H), 2.43-2.29 (m, | |
| | 2H), 2.03-1.91 (m, 2H). | |
| Example 276 | 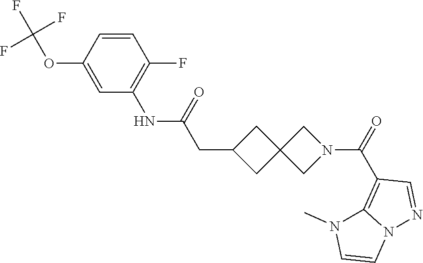 [SMILES: Cn1ccn2ncc(C(=O)N3CC4(CC(CC(=O)Nc5cc(OC(F)(F)F)ccc5F)C4)C3)c12] | 35 mg (40%) |
| | LCMS (ESI): Method 2, t_{R} = 4.37 min, m/z (M + 1) = 480.4 | |
| | ¹H NMR (400 MHz, DMSO-d6) δ 9.92 (s, 1H), 8.11-8.05 (m, 1H), 7.76 | |
| | (s, 1H), 7.76 (d, J = 2.1 Hz, 1H), 7.44-7.36 (m, 1H), 7.25 (s, 1H), 7.18- | |
| | 7.10 (m, 1H), 4.46-3.84 (m, 4H), 3.96 (s, 3H), 2.55-2.45 (m, 1H), 2.39- | |
| | 2.31 (m, 2H), 2.00-1.91 (m, 2H). | |
| Example 301 | 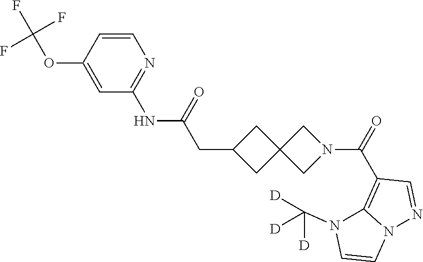 [SMILES: [2H]C([2H])([2H])n1ccn2ncc(C(=O)N3CC4(CC(CC(=O)Nc5cc(OC(F)(F)F)ccn5)C4)C3)c12] | 16 mg (21%) Preparative SFC (Method 20) |
| | LCMS (ESI): Method 11, t_{R} = 4.12 min, m/z (M + 1) = 466.4 | |
| | ¹H NMR (400 MHz, DMSO-d6) δ 10.84 (s, 1H), 8.42 (d, J = 5.9 Hz, 1H), | |
| | 8.09-8.09 (m, 1H), 7.76 (s, 1H), 7.65 (d, J = 2.3 Hz, 1H), 7.25-7.25 (m, | |
| | 1H), 7.12-7.10 (m, 1H), 4.49-3.92 (m, 4H), 2.53 (s, 2H), 2.37-2.32 | |
| | (m, 2H), 1.97-1.90 (m, 2H). | |
| Example 302 | 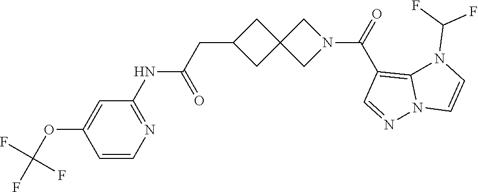 [SMILES: O=C(CC1CC2(C1)CN(C(=O)c1cnn3ccn(C(F)F)c13)C2)Nc1cc(OC(F)(F)F)ccn1] | 12 mg (25%) |
| | LCMS (ESI): Method 11, t_{R} = 4.78 min, m/z (M + 1) = 499.5 | |
| | ¹H NMR (400 MHz, CDCl₃) δ 10.84 (s, 1H), 8.75 (t, J = 60.1 Hz, 1H), | |
| | 8.42 (d, J = 5.6 Hz, 1H), 8.11-8.10 (m, 1H), 8.05 (d, J = 2.6 Hz, 1H), 7.94 | |
| | (s, 1H), 7.81 (d, J = 1.4 Hz, 1H), 7.12-7.10 (m, 1H), 4.50-4.39 (m, 2H), | |
| | 4.11-3.93 (m, 2H), 2.54 (s, 2H), 2.38-2.34 (m, 2H), 1.99-1.93 (m, | |
| | 2H). | |
| Intermediate 55 | 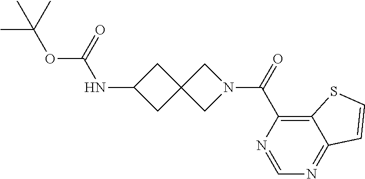 [SMILES: CC(C)(C)OC(=O)NC1CC2(C1)CN(C(=O)c1ncnc3ccsc13)C2] | 250mg (28%) |
| | ¹H NMR (400 MHz, DMSO-d6) δ 9.28 (d, J = 5.3 Hz, 1H), 8.59 (d, | |
| | J = 5.5 Hz, 1H), 7.68 (dd, J = 1.6, 5.6 Hz, 1H), 7.14 (d, J = 7.7 Hz, 1H), | |
| | 4.83 (s, 1H), 4.71 (s, 1H), 4.21 (s, 1H), 4.10 (s, 1H), 3.89-3.83 (m, 1H), | |
| | 2.16-2.08 (m, 2H), 1.38 (s, 9H). | |
| Intermediate 56 | 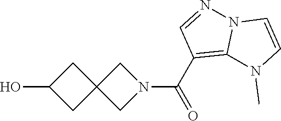 [SMILES: Cn1ccn2ncc(C(=O)N3CC4(CC(O)C4)C3)c12] | 90 mg (52%) |
| | LCMS (ESI): Method 1, t_{R} = 1.05 min-m/z (M + 1) = 261.1 | |
| Intermediate 135 | 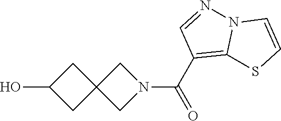 [SMILES: O=C(c1cnn2ccsc12)N1CC2(CC(O)C2)C1] | 504 mg (42%) |

¹H NMR (400 MHz, DMSO-d6) δ 8.31 (d, J = 4.1 Hz, 1H), 8.05 (s, 1H),
7.52 (dd, J = 1.4, 4.1 Hz, 1H), 5.12-5.08 (m, 1H), 4.49-4.20 (m, 2H),
4.06-3.89 (m, 3H), 2.49-2.44 (m, 2H), 2.04-1.97 (m, 2H).

### Step 2-(6-amino-2-azaspiro[3.3]heptan-2-yl) (pyrazolo[1,5-a]pyrazin-3-yl) methanone (Intermediate 10)

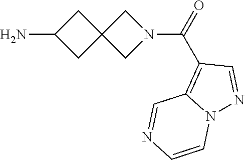 [SMILES: NC1CC2(C1)CN(C(=O)c1cnn3ccncc13)C2]

Intermediate 10 was prepared by following the procedure described for Intermediate 7.

SM: Intermediate 9: 750 mg (1 eq); TFA: 3.4 mL (20 eq)

Amount/yield: 460 mg, 1.79 mmol, 80%

LCMS (ESI): Method 1, t_{R}=0.94 min, m/z (M+1)=258

The following Intermediates were prepared analogously to what described for Intermediate 10, by starting from the suitable precursor (see Table 20).

TABLE 20

| | | Product Amount |
|---|---|---|
| | Structure | (Yield) |
| N° | Analytical Data | |
| Intermediate 57 | 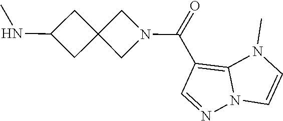 [SMILES: CNC1CC2(C1)CN(C(=O)c1cnn3ccn(C)c13)C2] | 230 mg (87%) |
| | LCMS (ESI): Method 1, t_{R} = 0.96 min, m/z (M + 1) = 274.1 | |
| Intermediate 58 | 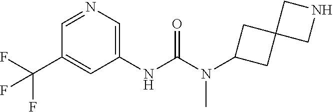 [SMILES: CN(C(=O)Nc1cncc(C(F)(F)F)c1)C1CC2(CNC2)C1] | 50 mg (73%) |
| | LCMS (ESI): Method 7, t_{R} = 0.39 min, m/z (M + 1) = 315.1 | |
| Intermediate 59 | 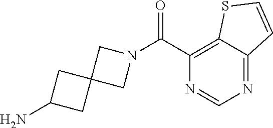 [SMILES: NC1CC2(C1)CN(C(=O)c1ncnc3ccsc13)C2] | 175 mg (96%) |
| | ¹H NMR (400 MHz, DMSO-d6) δ 9.28 (d, J = 1.8 Hz, 1H), 8.59 (d, J = 5.5 | |
| | Hz, 1H), 7.68 (d, J = 5.6 Hz, 1H), 4.78 (s, 1H), 4.69 (s, 1H), 4.17 (s, 1H), | |
| | 4.08 (s, 1H), 3.27-3.16 (m, 1H), 2.49-2.41 (m, 2H), −1.91-1.82 (m, 2H). | |
| Intermediate 182 | 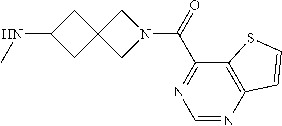 [SMILES: CNC1CC2(C1)CN(C(=O)c1ncnc3ccsc13)C2] | mg (96%) |

LCMS (ESI): Method 16, t_{R} = 1.57 min, m/z (M + 1) = 288.3

### Step 3—1-(3-fluoro-5-(trifluoromethyl)phenyl)-3-(2-(pyrazolo[1,5-a]pyrazine-3-carbonyl)-2-azaspiro[3.3]heptan-6-yl)urea (Example 16)

To a solution of Intermediate 10 (50 mg, 0.194 mmol) in DCM (1.00 mL) 1-fluoro-3-isocyanato-5-(trifluoromethyl)benzene (0.028 mL, 0.194 mmol) was added and the reaction mixture was stirred at rt for 4 h, then concentrated in vacuo. The residue was purified by preparative HPLC (Sunfire C18 19×150 mm, 10 μm 20-80% ACN/H₂O (10 mM NH₄CO₃), 20 mL/min, RT) to give the title compound (7.5 mg, 0.0162 mmol, 8% yield).

LCMS (ESI): Method 2, t_{R}=4.04 min, m/z (M+1)=463.2

¹H NMR (400 MHz, DMSO-d6) δ 9.55-9.54 (m, 1H), 9.06 (s, 1H), 8.91 (dd, J=1.1, 4.7 Hz, 1H), 8.44 (s, 1H), 8.11 (d, J=4.8 Hz, 1H), 7.62 (s, 1H), 7.57 (d, J=11.6 Hz, 1H), 7.14-7.10 (m, 1H), 6.83-6.79 (m, 1H), 4.58 (s, 1H), 4.48 (s, 1H), 4.15 (s, 1H), 4.07-4.02 (m, 2H), 2.60-2.54 (m, 2H), 2.22-2.14 (m, 2H).

The following Examples and Intermediates were prepared analogously to what described for Example 16, step 3, by starting from the suitable Intermediate and the suitable, commercially available isocyanate (see Table 21).

TABLE 21

| | | Product Amount |
|---|---|---|
| | Structure | (Yield) |
| N° | Analytical Data | |
| Example 17 | 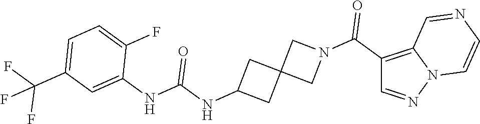 [SMILES: O=C(Nc1cc(C(F)(F)F)ccc1F)NC1CC2(C1)CN(C(=O)c1cnn3ccncc13)C2] | 16 mg (17%) |
| | LCMS (ESI): Method 2, t_{R} = 4.1 min, m/z (M + 1) = 463.5 ¹H NMR (400 MHz, DMSO-d6) δ 9.55 (d, J = 1.3 Hz, 1H), 8.91 (dd, J = 1.1, 4.7 Hz, 1H), 8.59-8.42 (m, 3H), 8.11 (d, J = 4.5 Hz, 1H), 7.46-7.40 (m, 1H), 7.33-7.28 (m, 1H), 7.02 (d, J = 7.1 Hz, 1H), 4.53 (d, J = 40.7 Hz, 1H), 4.15-3.97 (m, 2H), 2.62- 2.57 (m, 2H), 2.16-2.07 (m, 2H) | |
| Example 31 | 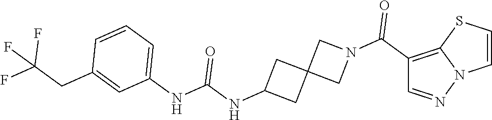 [SMILES: O=C(Nc1cccc(CC(F)(F)F)c1)NC1CC2(C1)CN(C(=O)c1cnn3ccsc13)C2] | 56 mg (43%) |
| | LCMS (ESI): Method 3, t_{R} = 4.06 min, m/z (M + 1) = 464.3 ¹H NMR (400 MHz, DMSO-d6) δ 8.45 (s, 1H), 8.31 (d, J = 4.0 Hz, 1H), 8.08 (s, 1H), 7.52 (dd, J = 1.3, 4.0 Hz, 1H), 7.40 (s, 1H), 7.37-7.33 (m, 1H), 7.21 (t, J = 7.8 Hz, 1H), 6.88 (d, J = 7.8 Hz, 1H), 6.43 (d, J = 7.6 Hz, 1H), 4.52-4.39 (m, 2H), 4.10-3.96 (m, 3H), 3.56 (q, J = 11.7 Hz, 2H), 2.58-2.52 (m, 2H), 2.16- 2.08 (m, 2H). | |
| Example 33 | 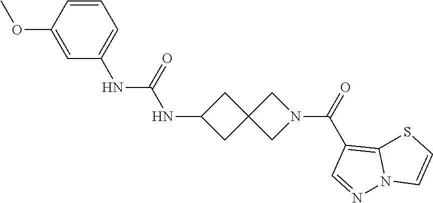 [SMILES: COc1cccc(NC(=O)NC2CC3(C2)CN(C(=O)c2cnn4ccsc24)C3)c1] | 52 mg (45%) |
| | LCMS (ESI): Method 3, t_{R} = 3.49 min, m/z (M + 1) = 412.2 ¹H NMR (400 MHz, DMSO-d6) δ 8.37 (s, 1H), 8.31 (d, J = 4.0 Hz, 1H), 8.07 (s, 1H), 7.52 (dd, J = 1.3, 4.0 Hz, 1H), 7.13-7.08 (m, 2H), 6.87-6.83 (m, 1H), 6.47 (dd, J = 2.5, 8.0 Hz, 1H), 6.42 (d, J = 7.1 Hz, 1H), 4.51-4.39 (m, 2H), 4.09-3.96 (m, 3H), 3.70 (s, 3H), 2.58-2.52 (m, 2H), 2.14-2.07 (m, 2H). | |
| Example 34 | 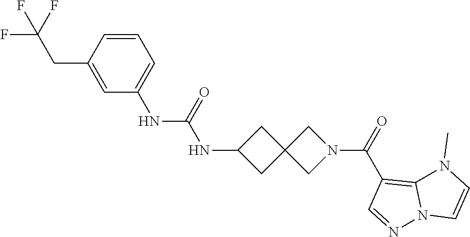 [SMILES: Cn1ccn2ncc(C(=O)N3CC4(CC(NC(=O)Nc5cccc(CC(F)(F)F)c5)C4)C3)c12] | 76 mg (57%) |
| | LCMS (ESI): Method 3, t_{R} = 3.96 min, m/z (M + 1) = 461.6 ¹H NMR (400 MHz, DMSO-d6) δ 8.44 (s, 1H), 7.77 (d, J = 1.0 Hz, 1H), 7.66 (d, J = 2.0 Hz, 1H), 7.39 (s, 1H), 7.35 (d, J = 8.1 Hz, 1H), 7.26-7.24 (m, 1H), 7.21 (t, J = 7.8 Hz, 1H), 6.87 (d, J = 7.3 Hz, 1H), 6.42 (d, J = 7.3 Hz, 1H), 4.25-3.96 (m, 8H), 3.56 (q, J = 11.6 Hz, 2H), 2.13-2.07 (m, 2H). | |
| Example 79 | 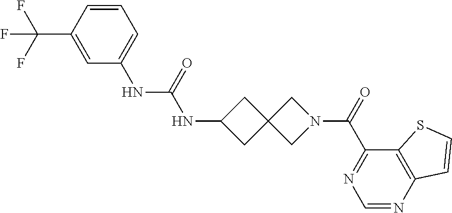 [SMILES: O=C(Nc1cccc(C(F)(F)F)c1)NC1CC2(C1)CN(C(=O)c1ncnc3ccsc13)C2] | 7 mg (8%) |
| | LCMS (ESI): Method 2, t_{R} = 4.57 min, m/z (M + 1) = 462.0 ¹H NMR (400 MHz, DMSO-d6) δ 9.29 (d, J = 8.0 Hz, 1H), 8.81 (d, J = 3.5 Hz, 1H), 8.59 (d, J = 5.5 Hz, 1H), 7.94 (d, J = 1.9 Hz, 1H), 7.68 (dd, J = 2.1, 5.6 Hz, 1H), 7.51 (d, J = 8.7 Hz, 1H), 7.44 (dd, J = 7.9, 7.9 Hz, 1H), 7.22 (d, J = 7.5 Hz, 1H), 6.64 (d, J = 7.4 Hz, 1H), 4.86 (s, 1H), 4.76 (s, 1H), 4.25 (s, 1H), 4.15 (s, 1H), 4.10-4.01 (m, 1H), 2.62-2.54 (m, 2H), 2.19-2.14 (m, 2H). | |
| Example 85 | 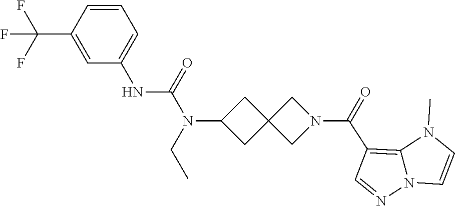 [SMILES: CCN(C(=O)Nc1cccc(C(F)(F)F)c1)C1CC2(C1)CN(C(=O)c1cnn3ccn(C)c13)C2] | 68 mg (72%) |
| | LCMS (ESI): Method 3, t_{R} = 4.32 min, m/z (M + 1) = 475.4 ¹H NMR (400 MHz, CDCl₃) δ 7.70 (s, 1H), 7.66 (s, 1H), 7.58 (d, J = 8.3 Hz, 1H), 7.40 (dd, J = 7.9, 7.9 Hz, 1H), 7.31-7.27 (m, 2H), 6.75-6.75 (m, 1H), 6.35 (s, 1H), 4.44-4.19 (m, 5H), 4.10 (s, 3H), 3.38 (q, J = 7.2 Hz, 2H), 2.67-2.60 (m, 2H), 2.43- 2.36 (m, 2H), 1.24 (dd, J = 7.2, 7.2 Hz, 3H). | |
| Example 86 | 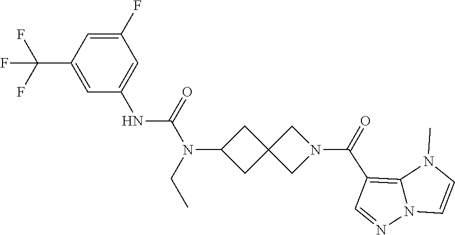 [SMILES: CCN(C(=O)Nc1cc(F)cc(C(F)(F)F)c1)C1CC2(C1)CN(C(=O)c1cnn3ccn(C)c13)C2] | 68 mg (69%) |
| | LCMS (ESI): Method 2, t_{R} = 4.58 min, m/z (M + 1) = 493.4 ¹H NMR (400 MHz, CDCl₃) δ 7.69 (s, 1H), 7.60-7.56 (m, 1H), 7.30 (d, J = 2.0 Hz, 2H), 6.98 (d, J = 8.2 Hz, 1H), 6.75-6.75 (m, 1H), 6.44 (s, 1H), 4.42-4.18 (m, 5H), 4.09 (s, 3H), 3.38 (q, J = 7.2 Hz, 2H), 2.67-2.60 (m, 2H), 2.43-2.36 (m, 2H), 1.24 (dd, J = 7.2, 7.2 Hz, 3H). | |
| Example 106 | 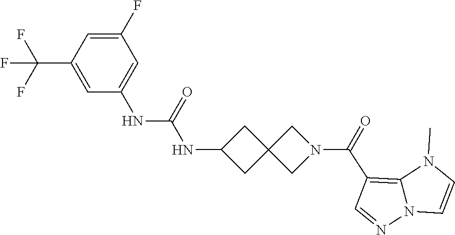 [SMILES: Cn1ccn2ncc(C(=O)N3CC4(CC(NC(=O)Nc5cc(F)cc(C(F)(F)F)c5)C4)C3)c12] | 73 mg (68%) |
| | LCMS (ESI): Method 2, t_{R} = 4.2 min, m/z (M + 1) = 465.4 ¹H NMR (400 MHz, DMSO-d6) δ 8.97 (s, 1H), 7.77 (s, 1H), 7.67-7.55 (m, 3H), 7.26 (s, 1H), 7.13 (d, J = 8.6 Hz, 1H), 6.71 (d, J = 7.8 Hz, 1H), 4.18-3.95 (m, 8H), 2.18-2.12 (m, 2H). | |
| Example 128 | 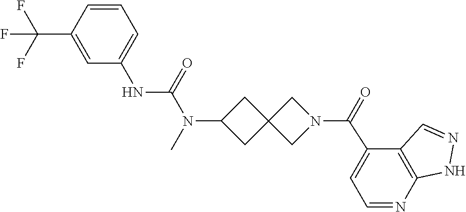 [SMILES: CN(C(=O)Nc1cccc(C(F)(F)F)c1)C1CC2(C1)CN(C(=O)c1ccnc3[nH]ncc13)C2] | 68 mg (56%) |
| | LCMS (ESI): Method 2, t_{R} = 3.95 min, m/z (M + 1) = 459.4 ¹H NMR (400 MHz, DMSO-d6) δ 13.87 (s, 1H), 8.62 (t, J = 4.4 Hz, 1H), 8.56 (d, J = 11.8 Hz, 1H), 8.24 (d, J = 2.7 Hz, 1H), 7.89 (d, J = 8.6 Hz,, 1H), 7.74 (t, J = 7.4 Hz, 1H), 7.49-7.41 (m, 1H), 7.33 (dd, J = 4.6, 9.4 Hz, 1H), 7.26 (d, J = 7.8 Hz, 1H), 4.62- 4.41 (m, 1H), 4.41-4.06 (m, 4H), 2.93-2.81 (m, 3H), 2.50-2.29 (m, 4H). | |
| Example 143 | 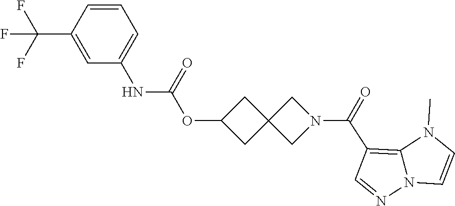 [SMILES: Cn1ccn2ncc(C(=O)N3CC4(CC(OC(=O)Nc5cccc(C(F)(F)F)c5)C4)C3)c12] | 67 mg (43%) |
| | LCMS (ESI): Method 3, t_{R} = 4.42 min, m/z (M + 1) = 448.4 ¹H NMR (400 MHz, DMSO-d6) δ 10.04 (s, 1H), 7.89 (s, 1H), 7.76 (d, J = 1.3 Hz, 1H), 7.69-7.65 (m, 2H), 7.52 (dd, J = 7.8, 7.8 Hz, 1H), 7.33 (d, J = 7.6 Hz, 1H), 7.26 (dd, J = 1.1, 2.1 Hz, 1H), 4.94-4.86 (m, 1H), 4.46-4.08 (m, 4H), 3.96 (s, 3H), 2.72-2.65 (m, 2H), 2.53 (d, J = 6.8 Hz, 1H), 2.34-2.27 (m, 2H). | |
| Intermediate 60 | 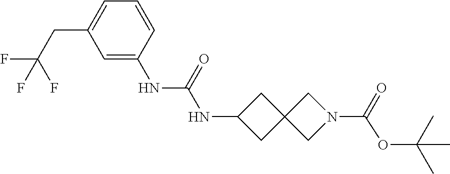 [SMILES: CC(C)(C)OC(=O)N1CC2(CC(NC(=O)Nc3cccc(CC(F)(F)F)c3)C2)C1] | 330 mg (quantitative) |

LCMS (ESI): Method 4, t_{R} = 1.70 min, m/z (M + 1-tBu) = 358.0

### Example 18: 1-(5-(tert-butyl) isoxazol-3-yl)-3-(2-(pyrazolo[1,5-a]pyrazine-3-carbonyl)-2-azaspiro[3.3]heptan-6-yl)urea

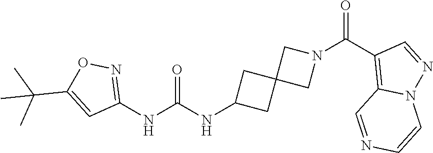 [SMILES: CC(C)(C)c1cc(NC(=O)NC2CC3(C2)CN(C(=O)c2cnn4ccncc24)C3)no1]

### Example 18

A solution of Intermediate 10 (50 mg, 0.194 mmol) and Intermediate 3 (51 mg, 0.194 mmol) in pyridine (1.00 mL) was stirred at 80° C. for 6 h. The reaction mixture was concentrated in vacuo and purified by preparative HPLC (Sunfire C18 19×150 mm, 10 μm 5-60% ACN/H₂O (10 mM NH₄CO₃), 20 mL/min, RT) to give the title compound (19 mg, 0.0458 mmol, 24% yield).

LCMS (ESI): Method 2, t_{R}=3.75 min, m/z (M+1)=424.2

¹H NMR (400 MHz, DMSO-d6) δ 9.55 (d, J=1.5 Hz, 1H), 9.26 (s, 1H), 8.91 (dd, J=1.3, 4.8 Hz, 1H), 8.44 (s, 1H), 8.11 (d, J=4.8 Hz, 1H), 6.74 (d, J=7.3 Hz, 1H), 6.33 (s, 1H), 4.59-4.44 (m, 2H), 4.15-4.01 (m, 3H), 2.59-2.52 (m, 2H), 2.19-2.11 (m, 2H), 1.27-1.26 (m, 9H).

The following Examples were prepared analogously to what described for Example 18 by starting from the suitable Intermediates (see Table 22).

TABLE 22

| | | Product Amount |
|---|---|---|
| | Structure | (Yield) |
| N° | Analytical Data | |
| Example 19 | 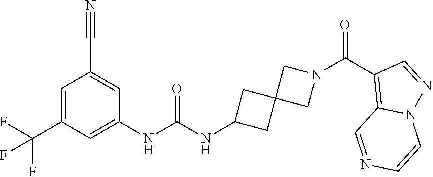 [SMILES: N#Cc1cc(NC(=O)NC2CC3(C2)CN(C(=O)c2cnn4ccncc24)C3)cc(C(F)(F)F)c1] | 28 mg (31%) |
| | LCMS (ESI): Method 2, t_{R} = 3.99 min, m/z (M + 1) = 470.2 ¹H NMR (400 MHz, DMSO-d6) δ 9.55 (d, J = 1.5 Hz, 1H), 9.08 (s, 1H), 8.91 (dd, J = 1.1, 4.7 Hz, 1H), 8.45 (s, 1H), 8.16-8.10 (m, 2H), 8.04 (s, 1H), 7.80 (s, 1H), 6.87 (d, J = 7.6 Hz, 1H), 4.59-4.47 (m, 2H), 4.17-4.02 (m, 3H), 2.60-2.53 (m, 2H), 2.23-2.16 (m, 2H). | |
| Example 53 | 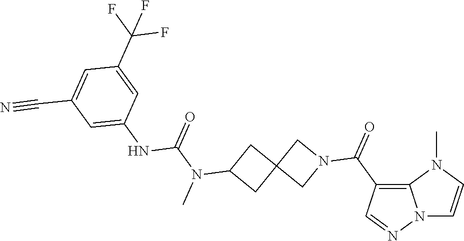 [SMILES: CN(C(=O)Nc1cc(C#N)cc(C(F)(F)F)c1)C1CC2(C1)CN(C(=O)c1cnn3ccn(C)c13)C2] | 72 mg (58%) |
| | LCMS (ESI): Method 2, t_{R} = 4.14 min, m/z (M + 1) = 468.4 ¹H NMR (400 MHz, DMSO-d6) δ 8.91 (s, 1H), 8.20 (d, J = 7.3 Hz, 2H), 7.84 (s, 1H), 7.76 (s, 1H), 7.66 (d, J = 2.0 Hz, 1H), 7.26 (dd, J = 1.3, 2.0 Hz, 1H), 4.57- 4.49 (m, 1H), 4.44-4.02 (m, 4H), 3.96 (s, 3H), 2.91 (s, 3H), 2.46-2.35 (m, 4H). | |
| Example 87 | 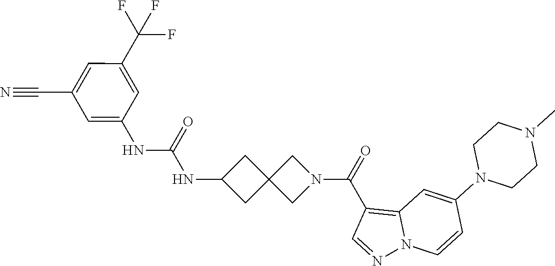 [SMILES: CN1CCN(c2ccn3ncc(C(=O)N4CC5(CC(NC(=O)Nc6cc(C#N)cc(C(F)(F)F)c6)C5)C4)c3c2)CC1] | 24 mg (35%) |

LCMS (ESI): Method 2, t_{R} = 3.35 min, m/z (M + 1) = 567.5 ¹H NMR (400 MHz, DMSO-d6) δ 9.07 (s, 1H), 8.53 (d, J = 7.8 Hz, 1H), 8.15 (s, 1H), 8.08 (s, 1H), 8.04 (s, 1H), 7.81 (s, 1H), 7.34 (d, J = 2.6 Hz, 1H), 6.97 (dd, J = 2.8, 7.8 Hz, 1H), 6.87 (d, J = 7.7 Hz, 1H), 4.47-3.91 (m, 5H), 3.31-3.26 (m, 4H), 2.58-2.53 (m, 4H), 2.27 (s, 3H), 2.21-2.13 (m, 2H).

### Example 20: 1-(5-(tert-butyl) isoxazol-3-yl)-3-(2-(pyrazolo[5,1-b]thiazole-7-carbonyl)-2-azaspiro[3.3]heptan-6-yl)urea

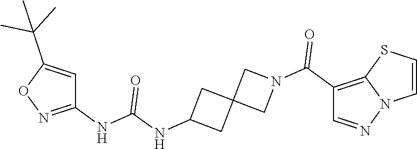 [SMILES: CC(C)(C)c1cc(NC(=O)NC2CC3(C2)CN(C(=O)c2cnn4ccsc24)C3)no1]

### Example 20

### Step 1 tert-butyl(2-(pyrazolo[5,1-b]thiazole-7-carbonyl)-2-azaspiro[3.3]heptan-6-yl)carbamate (Intermediate 11)

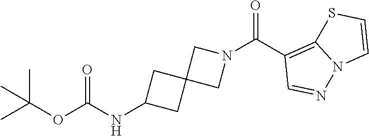 [SMILES: CC(C)(C)OC(=O)NC1CC2(C1)CN(C(=O)c1cnn3ccsc13)C2]

To a solution of tert-butyl(2-azaspiro[3.3]heptan-6-yl)carbamate (350 mg, 1.65 mmol), pyrazolo[5,1-b]thiazole-7-carboxylic acid (277 mg, 1.65 mmol) and TCFH (463 mg, 1.65 mmol) in DMF (2.00 mL) 1-methyl-1H-imidazole (0.13 mL, 1.65 mmol) was added and the reaction mixture was stirred at rt overnight. Water was added and the product was extracted into EtOAc. The organic phase was dried over a hydrophobic frit and concentrated in vacuo to give the title compound (467 mg, 1.29 mmol, 78% yield).

LCMS (ESI): Method 4, t_{R}=1.39 min, m/z (M+1)=363.1

The following Examples and Intermediate were prepared analogously to what described for Intermediate 11 by starting from the suitable Intermediates (see Table 23).

TABLE 23

| | | Product Amount |
|---|---|---|
| | Structure | (Yield) |
| N° | Analytical Data | |
| Example 60 | 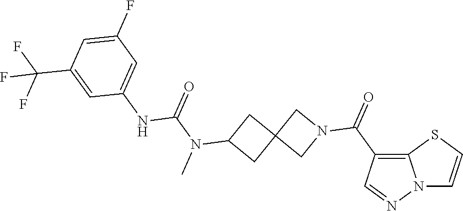 [SMILES: CN(C(=O)Nc1cc(F)cc(C(F)(F)F)c1)C1CC2(C1)CN(C(=O)c1cnn3ccsc13)C2] | 49 mg (40%) |
| | LCMS (ESI): Method 2, t_{R} = 4.49 min, m/z (M + 1) = 482.2 ¹H NMR (400 MHz, DMSO-d6) δ 8.80 (s, 1H), 8.31 (d, J = 4.3 Hz, 1H), 8.07 (s, 1H), 7.76-7.71 (m, 2H), 7.53 (dd, J = 1.5, 4.0 Hz, 1H), 7.17 (d, J = 8.6 Hz, 1H), 4.60-4.36 (m, 3H), 4.15-3.94 (m, 2H), 2.90 (s, 3H), 2.48-2.37 (m, 4H). | |
| Example 68 | 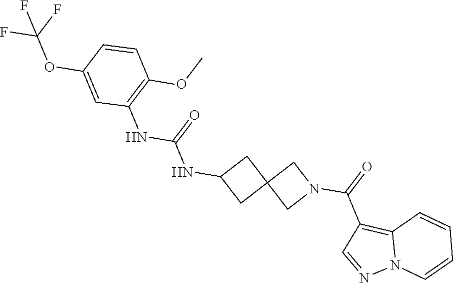 [SMILES: COc1ccc(OC(F)(F)F)cc1NC(=O)NC1CC2(C1)CN(C(=O)c1cnn3ccccc13)C2] | 26 mg (26%) |
| | LCMS (ESI): Method 2, t_{R} = 4.46 min, m/z (M + 1) = 490.3 ¹H NMR (400 MHz, DMSO-d6) δ 8.80 (d, J = 6.8 Hz, 1H), 8.29 (s, 1H), 8.21- 8.16 (m, 2H), 8.06 (s, 1H), 7.50-7.45 (m, 1H), 7.29 (d, J = 7.3 Hz, 1H), 7.11- 7.07 (m, 1H), 7.03 (d, J = 8.8 Hz, 1H), 6.84 (dd, J = 2.4, 8.5 Hz, 1H), 4.55-4.40 (m, 2H), 4.14-4.01 (m, 3H), 3.87 (s, 3H), 2.61-2.52 (m, 2H), 2.07 (s, 2H). | |
| Example 88 | 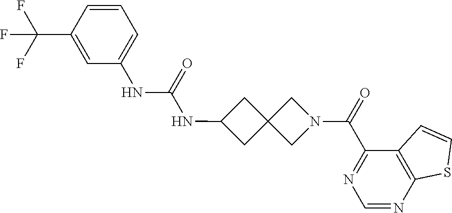 [SMILES: O=C(Nc1cccc(C(F)(F)F)c1)NC1CC2(C1)CN(C(=O)c1ncnc3sccc13)C2] | 32 mg (20%) |
| | LCMS (ESI): Method 2, t_{R} = 4.59 min, m/z (M + 1) = 462.2 ¹H NMR (400 MHz, DMSO-d6) δ 9.16 (d, J = 7.5 Hz, 1H), 8.73 (d, J = 4.4 Hz, 1H), 8.10 (d, J = 6.0 Hz, 1H), 7.97 (dd, J = 1.9, 6.1 Hz, 1H), 7.93 (s, 1H), 7.49 (d, J = 8.3 Hz, 1H), 7.44 (dd, J = 7.8, 7.8 Hz, 1H), 7.22 (d, J = 7.4 Hz, 1H), 6.55 (dd, J = 2.4, 7.6 Hz, 1H), 4.65 (s, 1H), 4.54 (s, 1H), 4.22 (s, 1H), 4.12 (s, 1H), 4.09- 3.97 (m, 1H), 2.60-2.52 (m, 2H), 2.19-2.07 (m, 2H). | |
| Example 93 | 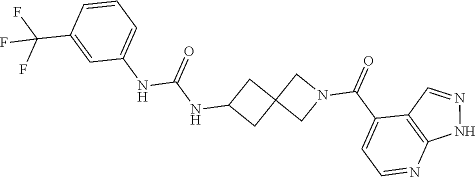 [SMILES: O=C(Nc1cccc(C(F)(F)F)c1)NC1CC2(C1)CN(C(=O)c1ccnc3[nH]ncc13)C2] | 10 mg (7%) |
| | LCMS (ESI): Method 2, t_{R} = 3.83 min, m/z (M + 1) = 445.3 ¹H NMR (400 MHz, DMSO-d6) δ 13.88 (br s, 1H), 8.77 (d, J = 13.1 Hz, 1H), 8.62 (dd, J = 4.9, 4.9 Hz, 1H), 8.24 (s, 1H), 7.93 (s, 1H), 7.50 (d, J = 8.6 Hz, 1H), 7.44 (dd, J = 7.9, 7.9 Hz, 1H), 7.33 (dd, J = 4.9, 4.9 Hz, 1H), 7.22 (d, J = 7.5 Hz, 1H), 6.61-6.53 (m, 1H), 4.36 (s, 1H), 4.25 (s, 1H), 4.22 (s, 1H), 4.10 (s, 1H), 4.09-3.94 (m, 1H), 2.60-2.53 (m, 2H), 2.18-2.07 (m, 2H). | |
| Example 96 | 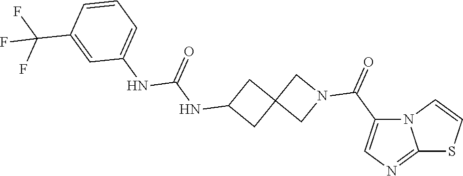 [SMILES: O=C(Nc1cccc(C(F)(F)F)c1)NC1CC2(C1)CN(C(=O)c1cnc3sccn13)C2] | 11 mg (7%) |
| | LCMS (ESI): Method 2, t_{R} = 4.06 min, m/z (M + 1) = 450.7 ¹H NMR (400 MHz, DMSO-d6) δ 8.79 (s, 1H), 8.26 (d, J = 4.5 Hz, 1H), 7.94 (s, 1H), 7.71 (s, 1H), 7.51 (d, J = 8.3 Hz, 1H), 7.47-7.41 (m, 2H), 7.22 (d, J = 7.5 Hz, 1H), 6.62 (d, J = 7.5 Hz, 1H), 4.49-4.49 (m, 1H), 4.37 (s, 1H), 4.16-3.97 (m, 3H), 2.59-2.52 (m, 2H), 2.19-2.11 (m, 2H). | |
| Example 99 | 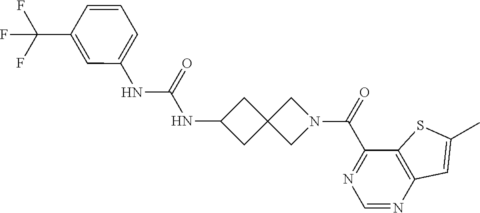 [SMILES: Cc1cc2ncnc(C(=O)N3CC4(CC(NC(=O)Nc5cccc(C(F)(F)F)c5)C4)C3)c2s1] | 8 mg (5%) |
| | LCMS (ESI): Method 2, t_{R} = 4.76 min, m/z (M + 1) = 476.3 ¹H NMR (400 MHz, DMSO-d6) δ 9.19 (d, J = 8.0 Hz, 1H), 8.86 (d, J = 4.9 Hz, 1H), 7.97-7.94 (m, 1H), 7.52 (d, J = 8.4 Hz, 1H), 7.45 (t, J = 8.0 Hz, 1H), 7.40 (s, 1H), 7.22 (d, J = 7.6 Hz, 1H), 6.70-6.66 (m, 1H), 4.86 (s, 1H), 4.75 (s, 1H), 4.24 (s, 1H), 4.14 (s, 1H), 4.10-4.01 (m, 1H), 2.70 (s, 3H), 2.61-2.54 (m, 2H), 2.21- 2.12 (m, 2H). | |
| Example 115 | 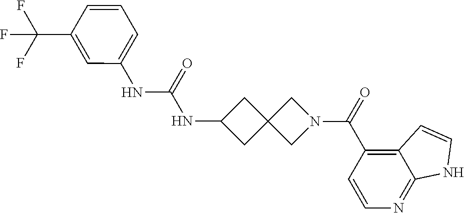 [SMILES: O=C(Nc1cccc(C(F)(F)F)c1)NC1CC2(C1)CN(C(=O)c1ccnc3[nH]ccc13)C2] | 107 mg (47%) |
| | LCMS (ESI): Method 2, t_{R} = 3.90 min, m/z (M + 1) = 444.5 ¹H NMR (400 MHz, DMSO-d6) δ 11.89-11.87 (m, 1H), 8.77 (d, J = 12.9 Hz, 1H), 8.28 (t, J = 4.3, 4.3 Hz, 1H), 7.92 (s, 1H), 7.59 (d, J = 3.4 Hz, 1H), 7.49 (d, J = 8.2 Hz, 1H), 7.44 (t, J = 7.8, 7.8 Hz, 1H), 7.22 (d, J = 7.4 Hz, 1H), 7.16 (t, J = 4.8, 4.8 Hz, 1H), 6.62-6.54 (m, 2H), 4.20 (d, J = 16.3 Hz, 2H), 4.09 (d, J = 11.24 Hz, 2H), 4.04-3.94 (m, 1H) 2.18-2.04 (m, 2H). | |
| Example 122 | 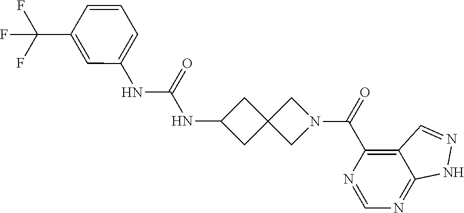 [SMILES: O=C(Nc1cccc(C(F)(F)F)c1)NC1CC2(C1)CN(C(=O)c1ncnc3[nH]ncc13)C2] | 38 mg (17%) |
| | LCMS (ESI): Method 2, t_{R} = 3.98 min, m/z (M + 1) = 446.2 ¹H NMR (400 MHz, DMSO-d6) δ 14.3 (s, 1H), 9.06 (d, J = 8.0 Hz, 1H), 8.75 (d, J = 3.4 Hz, 1H), 8.51 (d, J = 2.0 Hz, 1H), 7.94-7.92 (m, 1H), 7.50 (d, J = 8.5 Hz, 1H), 7.44 (t, J = 7.9 Hz, 1H), 7.22 (d, J = 7.5 Hz, 1H), 6.56 (d, J = 7.5 Hz, 1H), 4.75 (s, 1H), 4.64 (s, 1H), 4.23 (s, 1H), 4.12 (s, 1H), 4.09-3.99 (m, 1H), 2.60-2.53 (m, 2H), 2.19-2.10 (m, 2H). | |
| Example 130 | 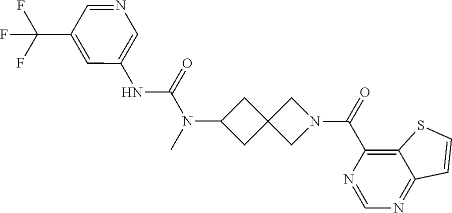 [SMILES: CN(C(=O)Nc1cncc(C(F)(F)F)c1)C1CC2(C1)CN(C(=O)c1ncnc3ccsc13)C2] | 18 mg (23%) |
| | LCMS (ESI): Method 2, t_{R} = 4.19 min, m/z (M + 1) = 477.5 ¹H NMR (400 MHz, DMSO-d6) δ 9.30 (d, J = 5.9 Hz, 1H), 8.95 (s, 1H), 8.84 (d, J = 6.3 Hz, 1H), 8.60 (d, J = 5.5 Hz, 1H), 8.52 (s, 1H), 8.32 (t, J = 2.4 Hz, 1H), 7.69 (dd, J = 1.8, 5.6 Hz, 1H), 4.92 (s, 1H), 4.78 (s, 1H), 4.63-4.54 (m, 1H), 4.30 (s, 1H), 4.17 (s, 1H), 2.93 (s, 3H), 2.48-2.43 (m, 4H). | |
| Example 139 | 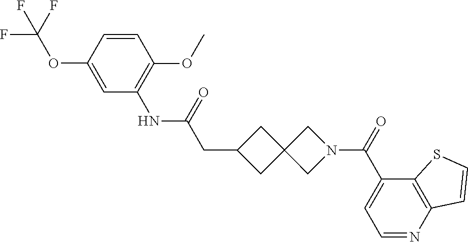 [SMILES: COc1ccc(OC(F)(F)F)cc1NC(=O)CC1CC2(C1)CN(C(=O)c1ccnc3ccsc13)C2] | 86 mg (56%) |
| | LCMS (ESI): Method 3, t_{R} = 4.76 min, m/z (M + 1) = 506.2 ¹H NMR (400 MHz, DMSO-d6) δ 9.24 (d, J = 6.6 Hz, 1H), 8.79-8.75 (m, 1H), 8.21 (d, J = 5.6 Hz, 1H), 8.11-8.08 (m, 1H), 7.60 (d, J = 5.6 Hz, 1H), 7.57-7.53 (m, 1H), 7.10 (d, J = 8.8 Hz, 1H), 7.03 (dd, J = 2.8, 8.9 Hz, 1H), 4.57 (s, 1H), 4.45 (s, 1H), 4.20 (s, 1H), 4.10 (s, 1H), 3.86 (d, J = 5.1 Hz, 3H), 2.41-2.34 (m, 2H), 2.00-1.93 (m, 2H). | |
| Example 140 | 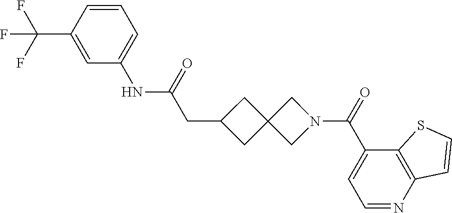 [SMILES: O=C(CC1CC2(C1)CN(C(=O)c1ccnc3ccsc13)C2)Nc1cccc(C(F)(F)F)c1] | 77 mg (52%) |
| | LCMS (ESI): Method 3, t_{R} = 4.52 min, m/z (M + 1) = 460.2 ¹H NMR (400 MHz, DMSO-d6) δ 10.19 (d, J = 5.8 Hz, 1H), 8.79-8.75 (m, 1H), 8.21 (d, J = 5.6 Hz, 1H), 8.08 (s, 1H), 7.75 (d, J = 8.6 Hz, 1H), 7.60 (d, J = 5.6 Hz, 1H), 7.57-7.50 (m, 2H), 7.37 (d, J = 7.8 Hz, 1H), 4.57 (s, 1H), 4.46 (s, 1H), 4.21 (s, 1H), 4.11 (s, 1H), 2.61-2.53 (m, 1H), 2.47-2.35 (m, 4H), 2.02-1.94 (m, 2H). | |
| Example 153 | 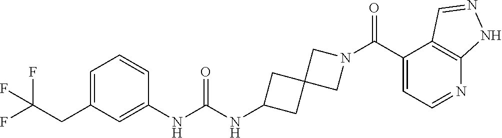 [SMILES: O=C(Nc1cccc(CC(F)(F)F)c1)NC1CC2(C1)CN(C(=O)c1ccnc3[nH]ncc13)C2] | 46 mg (37%) |
| | LCMS (ESI): Method 2, t_{R} = 3.75 min, m/z (M + 1) = 459.2 ¹H NMR (400 MHz, DMSO-d6) δ 13.86 (s, 1H), 8.61 (t, J = 4.8 Hz, 1H), 8.41 (d, J = 12.6 Hz, 1H), 8.23 (s, 1H), 7.39-7.30 (m, 3H), 7.20 (t, J = 7.8 Hz, 1H), 6.87 (d, J = 7.4 Hz, 1H), 6.38 (t, J = 6.8 Hz, 1H), 4.35 (s, 1H), 4.24-4.19 (m, 2H), 4.10- 3.93 (m, 2H), 3.55 (q, J = 11.6 Hz, 2H), 2.14-2.04 (m, 2H). | |
| Example 154 | 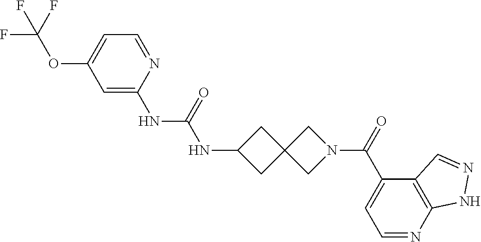 [SMILES: O=C(Nc1cc(OC(F)(F)F)ccn1)NC1CC2(C1)CN(C(=O)c1ccnc3[nH]ncc13)C2] | 38 mg (26%) |
| | LCMS (ESI): Method 2, t_{R} = 3.67 min, m/z (M + 1) = 462.6 ¹H NMR (400 MHz, DMSO-d6) δ 13.87 (s, 1H), 9.32 (d, J = 12.3 Hz, 1H), 8.61 (t, J = 5.0 Hz, 1H), 8.31-8.26 (m, 1H), 8.23 (s, 1H), 7.69-7.54 (m, 2H), 7.32 (t, J = 5.3 Hz, 1H), 6.93 (s, 1H), 4.35 (s, 1H), 4.22 (d, J = 11.5 Hz, 2H), 4.10-3.98 (m, 2H), 2.61-2.54 (m, 2H), 2.17-2.08 (m, 2H). | |
| Example 160 | 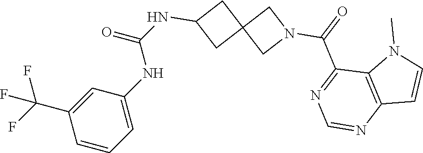 [SMILES: Cn1ccc2ncnc(C(=O)N3CC4(CC(NC(=O)Nc5cccc(C(F)(F)F)c5)C4)C3)c21] | 38 mg (26%) |
| | LCMS (ESI): Method 2, t_{R} = 3.90 min, m/z (M + 1) = 459.4 ¹H NMR (400 MHz, DMSO-d6) δ 8.82 (d, J = 4.5 Hz, 1H), 8.72 (d, J = 11.8 Hz, 1H), 7.97-7.91 (m, 2H), 7.51-7.40 (m, 2H), 7.21 (d, J = 7.6 Hz, 1H), 6.70 (t, J = 3.1 Hz, 1H), 6.54 (t, J = 7.0 Hz, 1H), 4.29-3.94 (m, 5H), 3.88 (s, 3H), 2.60- 2.54 (m, 2H), 2.20-2.07 (m, 2H). | |
| Example 209 | 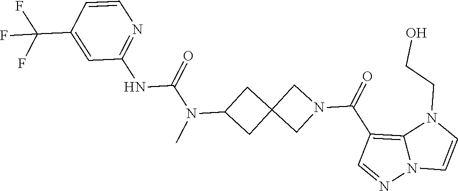 [SMILES: CN(C(=O)Nc1cc(C(F)(F)F)ccn1)C1CC2(C1)CN(C(=O)c1cnn3ccn(CCO)c13)C2] | 15 mg (8%) |
| | LCMS (ESI): Method 2, t_{R} = 3.63 min, m/z (M + 1) = 492.4 ¹H NMR (400 MHz, DMSO-d6) δ 9.42 (s, 1H), 8.50 (d, J = 5.2 Hz, 1H), 8.13 (s, 1H), 7.77 (s, 1H), 7.65 (d, J = 2.2 Hz, 1H), 7.31 (dd, J = 1.0, 5.3 Hz, 1H), 7.26 (dd, J = 1.2, 2.2 Hz, 1H), 4.89 (t, J = 5.2 Hz, 1H), 4.64-4.50 (m, 1H), 4.47 (t, J = 5.3 Hz, 2H), 4.46-3.79 (m, 4H), 3.64 (q, J = 5.2 Hz, 2H), 2.90 (s, 3H), 2.47-2.30 (m, 4H). | |
| Example 210 | 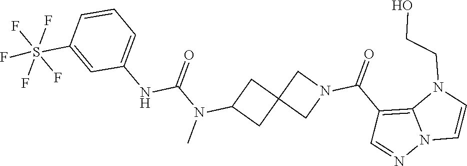 [SMILES: CN(C(=O)Nc1cccc(S(F)(F)(F)(F)F)c1)C1CC2(C1)CN(C(=O)c1cnn3ccn(CCO)c13)C2] | 10.5 mg (5%) |
| | LCMS (ESI): Method 2, t_{R} = 4.16 min, m/z (M + 1) = 549.4 ¹H NMR (400 MHz, DMSO-d6) δ 8.68 (s, 1H), 8.14-8.10 (m, 1H), 7.78 (s, 2H), 7.75 (d, J = 7.8 Hz, 1H), 7.65 (d, J = 2.2 Hz, 1H), 7.50-7.40 (m, 2H), 7.28- 7.24 (m, 1H), 4.90 (br s, 1H), 4.63-4.50 (m, 1H), 4.47 (t, J = 5.3 Hz, 2H), 4.44- 3.83 (m, 4H), 3.70-3.60 (m, 2H), 2.90 (s, 3H), 2.46-2.34 (m, 4H). | |
| Example 211 | 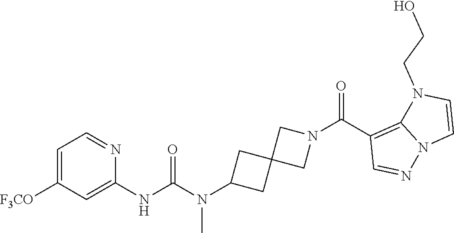 [SMILES: CN(C(=O)Nc1cc(OC(F)(F)F)ccn1)C1CC2(C1)CN(C(=O)c1cnn3ccn(CCO)c13)C2] | 9 mg (5%) |
| | LCMS (ESI): Method 2, t_{R} = 3.58 min, m/z (M + 1) = 508.4 ¹H NMR (400 MHz, DMSO-d6) δ 9.31 (s, 1H), 8.34 (d, J = 5.6 Hz, 1H), 7.84 (s, 1H), 7.77 (s, 1H), 7.65 (d, J = 2.2 Hz, 1H), 7.28-7.23 (m, 1H), 6.98 (d, J = 5.6 Hz, 1H), 4.90 (t, J = 5.2 Hz, 1H), 4.63-4.51 (m, 1H), 4.47 (t, J = 5.3 Hz, 2H), 4.44- 3.69 (m, 4H), 3.65 (q, J = 5.2 Hz, 2H), 2.89 (s, 3H), 2.46-2.28 (m, 4H). | |
| Example 231 | 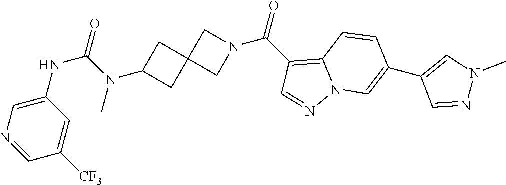 [SMILES: CN(C(=O)Nc1cncc(C(F)(F)F)c1)C1CC2(C1)CN(C(=O)c1cnn3cc(-c4cnn(C)c4)ccc13)C2] | 14 mg (22%) |
| | LCMS (ESI): Method 5, t_{R} = 5.54 min, m/z (M + 1) = 539.50 ¹H NMR (400 MHz, DMSO-d6) δ ppm 9.07 (1 H, s), 8.91 (1 H, d, J = 2.19 Hz), 8.82 (1 H, s), 8.48 (1 H, s), 8.27 (1 H, s), 8.25 (1 H, s), 8.22 (1 H, s), 8.14 (1 H, d, J = 9.15 Hz), 8.00 (1 H, s), 7.72 (1 H, dd, J = 9.21, 1.32 Hz), 4.52 (3 H, m), 4.07 (2 H, m), 3.85 (3 H, s), 2.89 (3 H, s), 2.39 (4 H, m). | |
| Example 240 | 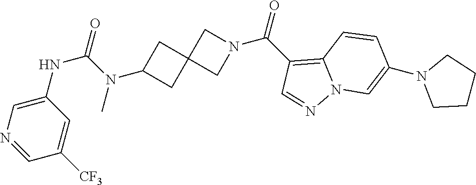 [SMILES: CN(C(=O)Nc1cncc(C(F)(F)F)c1)C1CC2(C1)CN(C(=O)c1cnn3cc(N4CCCC4)ccc13)C2] | 11 mg (10%) |
| | LCMS (ESI): Method 5, t_{R} = 6.48 min, m/z (M + 1) = 528.40 ¹H NMR (400 MHz, DMSO-d6) δ ppm 8.94 (1 H, d, J = 1.97 Hz), 8.85 (1 H, s), 8.52 (1 H, s), 8.31 (1 H, s), 8.04 (2 H, d, J = 9.43 Hz), 7.86 (1 H, s), 7.23 (1 H, dd, J = 9.76, 1.86 Hz), 4.57 (3 H, br t, J = 8.55 Hz), 4.07 (2 H, m), 3.26 (4 H, m), 2.92 (3 H, s), 2.42 (4 H, m), 1.97 (4 H, m). | |
| Example 252 | 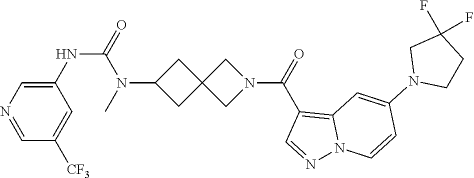 [SMILES: CN(C(=O)Nc1cncc(C(F)(F)F)c1)C1CC2(C1)CN(C(=O)c1cnn3ccc(N4CCC(F)(F)C4)cc13)C2] | 9 mg (4%) |
| | LCMS (ESI): Method 5, t_{R} = 5.51 min, m/z (M + 1) = 564.26 ¹H NMR (600 MHz, DMSO-d6) δ ppm 8.94 (1 H, d, J = 2.35 Hz), 8.85 (1 H, s), 8.57 (1 H, d, J = 7.63 Hz), 8.52 (1 H, s), 8.31 (1 H, t, J = 1.98 Hz), 8.06 (1 H, s), 7.02 (1 H, d, J = 2.64 Hz), 6.66 (1 H, dd, J = 7.70, 2.71 Hz), 4.57 (1 H, m), 4.21 (4 H, m), 3.84 (2 H, t, J = 13.06 Hz), 3.59 (2 H, t, J = 7.26 Hz), 2.92 (3 H, s), 2.60 (2 H, m), 2.41 (4 H, m). | |
| Example 275 | 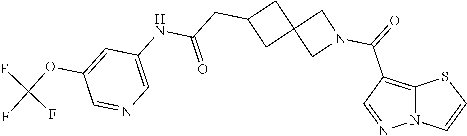 [SMILES: O=C(CC1CC2(C1)CN(C(=O)c1cnn3ccsc13)C2)Nc1cncc(OC(F)(F)F)c1] | 14 mg (23%) |
| | LCMS (ESI): Method 2, t_{R} = 3.93 min, m/z (M + 1) = 466.3 ¹H NMR (400 MHz, DMSO-d6) δ 10.44 (s, 1H), 8.67 (d, J = 2.0 Hz, 1H), 8.34 (d, J = 2.1 Hz, 1H), 8.31 (d, J = 4.1 Hz, 1H), 8.24 (s, 1H), 8.06 (s, 1H), 7.52 (dd, J = 1.1, 4.1 Hz, 1H), 4.56-4.21 (m, 2H), 4.21-3.83 (m, 2H), 2.62- 2.45 (m, 1H), 2.43-2.29 (m, 2H), 2.03-1.91 (m, 2H). | |
| Intermediate 61 | 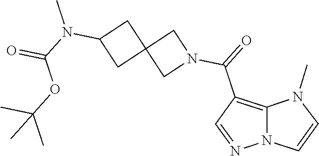 [SMILES: CN(C(=O)OC(C)(C)C)C1CC2(C1)CN(C(=O)c1cnn3ccn(C)c13)C2] | 360 mg (76%) |
| | LCMS (ESI): Method 1, t_{R} = 1.49 min, m/z (M + 1) = 374.2 | |
| Intermediate 62 | 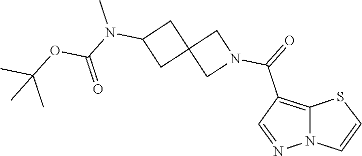 [SMILES: CN(C(=O)OC(C)(C)C)C1CC2(C1)CN(C(=O)c1cnn3ccsc13)C2] | 120 mg (33%) |
| | LCMS (ESI): Method 1, t_{R} = 1.53 min, m/z (M + 1) = 377.1 | |
| Intermediate 63 | 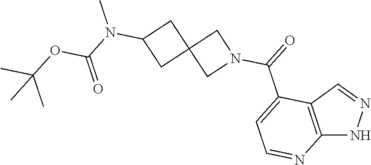 [SMILES: CN(C(=O)OC(C)(C)C)C1CC2(C1)CN(C(=O)c1ccnc3[nH]ncc13)C2] | 117 mg (66%) |
| | LCMS (ESI): Method 4, t_{R} = 1.37 min, m/z (M + 1) = 372.3 | |
| Intermediate 64 | 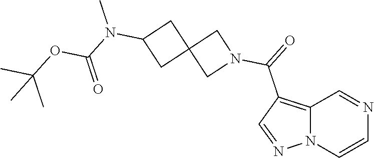 [SMILES: CN(C(=O)OC(C)(C)C)C1CC2(C1)CN(C(=O)c1cnn3ccncc13)C2] | 479 mg (69%) |
| | LCMS (ESI): Method 1, t_{R} = 1.46 min, m/z (M + 1) = 372.1 | |
| Intermediate 65 | 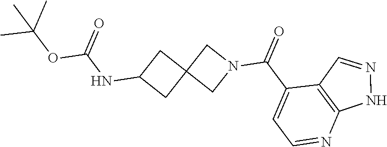 [SMILES: CC(C)(C)OC(=O)NC1CC2(C1)CN(C(=O)c1ccnc3[nH]ncc13)C2] | 108 mg (quantitative) |
| | LCMS (ESI): Method 4, t_{R} = 1.1 min, m/z (M + 1) = 358.2 | |
| Intermediate 136 | 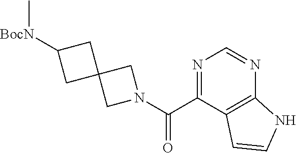 [SMILES: CN(C(=O)OC(C)(C)C)C1CC2(C1)CN(C(=O)c1ncnc3[nH]ccc13)C2] | 263 mg (37%) |
| | LCMS (ESI): Method 1, t_{R} = 1.49 min, m/z (M + 1) = 472.1 | |
| Intermediate 137 | 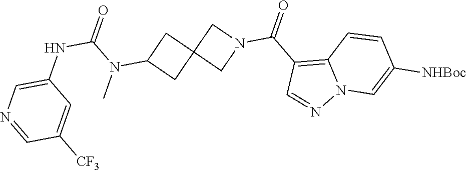 [SMILES: CN(C(=O)Nc1cncc(C(F)(F)F)c1)C1CC2(C1)CN(C(=O)c1cnn3cc(NC(=O)OC(C)(C)C)ccc13)C2] | 150 mg (73%) |
| | LCMS (ESI): Method 7, t_{R} = 1.04 min, m/z (M + 1) = 574.3 | |
| Intermediate 138 | 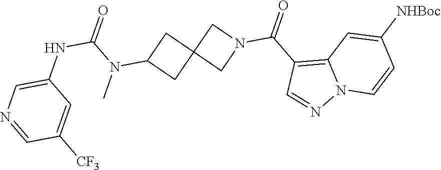 [SMILES: CN(C(=O)Nc1cncc(C(F)(F)F)c1)C1CC2(C1)CN(C(=O)c1cnn3ccc(NC(=O)OC(C)(C)C)cc13)C2] | 50 mg (25%) |
| | LCMS (ESI): Method 7, t_{R} = 1.03 min, m/z (M + 1) = 574.2 | |
| Intermediate 139 | 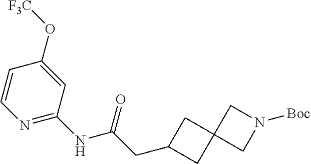 [SMILES: CC(C)(C)OC(=O)N1CC2(CC(CC(=O)Nc3cc(OC(F)(F)F)ccn3)C2)C1] | 320 mg (98%) |
| | LCMS (ESI): Method 7, t_{R} = 1.23 min, m/z (M + 1) = 414.2 | |
| Intermediate 140 | 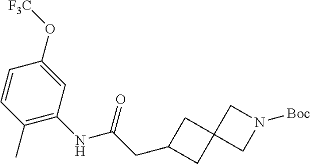 [SMILES: Cc1ccc(OC(F)(F)F)cc1NC(=O)CC1CC2(C1)CN(C(=O)OC(C)(C)C)C2] | 200 mg (60%) |
| | LCMS (ESI): Method 7, t_{R} = 1.28 min, m/z (M + 1) = 427.3 | |
| Intermediate 141 | 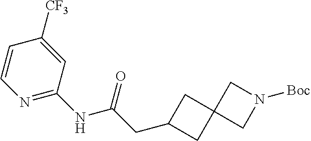 [SMILES: CC(C)(C)OC(=O)N1CC2(CC(CC(=O)Nc3cc(C(F)(F)F)ccn3)C2)C1] | 313 mg (100%) |

LCMS (ESI): Method 7, t_{R} = 1.22 min, m/z (M + 1) = 398.3

### Step 2-(6-amino-2-azaspiro[3.3]heptan-2-yl) (pyrazolo[5,1-b]thiazol-7-yl) methanone (Intermediate 12)

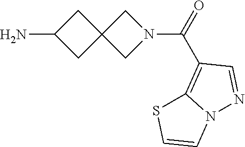 [SMILES: NC1CC2(C1)CN(C(=O)c1cnn3ccsc13)C2]

Intermediate 12 was prepared by following the procedure described for the synthesis of Intermediate 7.

SM: Intermediate 11: 467 mg (1 eq); TFA: 0.99 mL (10 eq)

Amount/yield: 314 mg, 1.20 mmol, 93%

LCMS (ESI): Method 4, t_{R}=0.98 min, m/z (M+1)=263.1

The following Intermediates were prepared analogously to what described for Intermediate 12, starting from suitable intermediate (see Table 24).

TABLE 24

| | | Product Amount |
|---|---|---|
| | Structure | (Yield) |
| N° | Analytical Data | |
| Intermediate 66 | 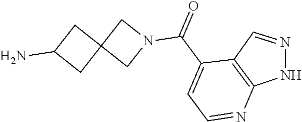 [SMILES: NC1CC2(C1)CN(C(=O)c1ccnc3[nH]ncc13)C2] | 78 mg (quantitative) |
| | LCMS (ESI): Method 4, t_{R} = 0.92 min, m/z (M + 1) = 258.2 | |
| Intermediate 67 | 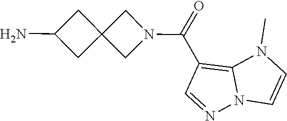 [SMILES: Cn1ccn2ncc(C(=O)N3CC4(CC(N)C4)C3)c12] | 1.07 g (64%) |
| | LCMS (ESI): Method 1, t_{R} = 0.93 min, m/z (M + 1) = 260.1 | |
| Intermediate 68 | 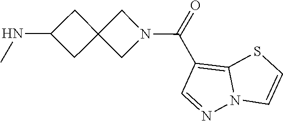 [SMILES: CNC1CC2(C1)CN(C(=O)c1cnn3ccsc13)C2] | 80 mg (91%) |
| | LCMS (ESI): Method 1, t_{R} = 0.97 min, m/z (M + 1) = 277.0 | |
| Intermediate 69 | 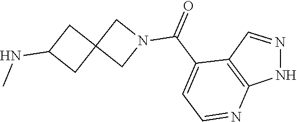 [SMILES: CNC1CC2(C1)CN(C(=O)c1ccnc3[nH]ncc13)C2] | 71 mg (83%) |
| | LCMS (ESI): Method 4, t_{R} = 0.98 min, m/z (M + 1) = 272.2 | |
| Intermediate 70 | 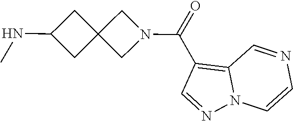 [SMILES: CNC1CC2(C1)CN(C(=O)c1cnn3ccncc13)C2] | 37 mg (72%) |
| | LCMS (ESI): Method 1, t_{R} = 0.92 min, m/z (M + 1) = 272.1 | |
| Intermediate 71 | 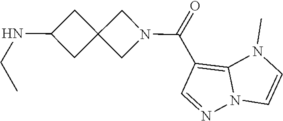 [SMILES: CCNC1CC2(C1)CN(C(=O)c1cnn3ccn(C)c13)C2] | 115 mg (93%) |
| | LCMS (ESI): Method 1, t_{R} = 1.00 min, m/z (M + 1) = 288.1 | |
| Intermediate 72 | 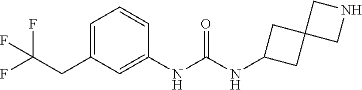 [SMILES: O=C(Nc1cccc(CC(F)(F)F)c1)NC1CC2(CNC2)C1] | 200 mg (80%) |
| | LCMS (ESI): Method 1, t_{R} = 1.15 min, m/z (M + 1) = 314.0 | |
| Intermediate 73 | 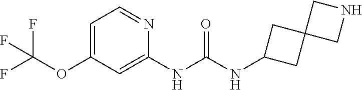 [SMILES: O=C(Nc1cc(OC(F)(F)F)ccn1)NC1CC2(CNC2)C1] | 100 mg (82%) |
| | LCMS (ESI): Method 1, t_{R} = 1.13 min, m/z (M + 1) = 317.0 | |
| Intermediate 142 | 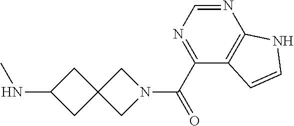 [SMILES: CNC1CC2(C1)CN(C(=O)c1ncnc3[nH]ccc13)C2] | 200 mg (39%) |
| | LCMS (ESI): Method 1, t_{R} = 0.78 min, m/z (M + 1) = 272.1 | |
| Intermediate 183 | 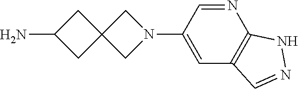 [SMILES: NC1CC2(C1)CN(c1cnc3[nH]ncc3c1)C2] | 510 mg (53%) |

LCMS (ESI): Method 16, t_{R} = 0.92 min, m/z (M + 1) = 230.1

### Step 3—1-(5-(tert-butyl) isoxazol-3-yl)-3-(2-(pyrazolo[5,1-b]thiazole-7-carbonyl)-2-azaspiro[3.3]heptan-6-yl)urea (Example 20)

Example 20 was prepared by following the procedure described for the synthesis of Intermediate 18.

SM: Intermediate 12: 50 mg (1 eq); Intermediate 3: 50 mg (1 eq)

Amount/yield: 34 mg, 0.0799 mmol, 42%

LCMS (ESI): Method 2, t_{R}=3.97 min, m/z (M+1)=429.3

¹H NMR (400 MHz, DMSO-d6) δ 9.26 (s, 1H), 8.31 (d, J=4.3 Hz, 1H), 8.06 (s, 1H), 7.52 (dd, J=1.4, 4.2 Hz, 1H), 6.74 (d, J=7.6 Hz, 1H), 6.33 (s, 1H), 4.44 (d, J=44.9 Hz, 2H), 4.09-3.94 (m, 3H), 2.58-2.53 (m, 2H), 2.17-2.07 (m, 2H), 1.26 (s, 9H).

### Example 21: 1-(2-(pyrazolo[5,1-b]thiazole-7-carbonyl)-2-azaspiro[3.3]heptan-6-yl)-3-(3-(trifluoromethoxy)phenyl)urea

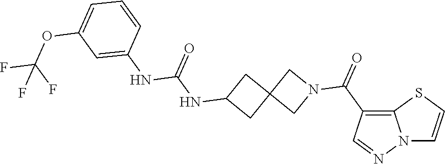 [SMILES: O=C(Nc1cccc(OC(F)(F)F)c1)NC1CC2(C1)CN(C(=O)c1cnn3ccsc13)C2]

### Example 21

### Step 1-phenyl(2-(pyrazolo[5,1-b]thiazole-7-carbonyl)-2-azaspiro[3.3]heptan-6-yl)carbamate (Intermediate 13)

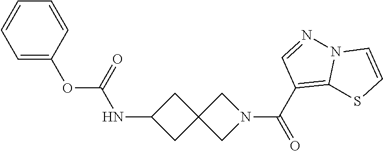 [SMILES: O=C(NC1CC2(C1)CN(C(=O)c1cnn3ccsc13)C2)Oc1ccccc1]

To a suspension of Intermediate 12 (250 mg, 0.953 mmol) and Na₂CO₃ (61 mg, 0.572 mmol) in EtOAc (20.00 mL), THF (4.00 mL) and water (4.00 mL) cooled to 0° C., phenyl chloroformate (0.13 mL, 1.05 mmol) was added dropwise. The resulting suspension was stirred at 0° C. for 20 min, then warmed up to rt. DMF (4 mL) was added and the reaction stirred for a further 16 h at rt. Water was added and product extracted into EtOAc. The organic phase was dried over a hydrophobic frit and concentrated in vacuo to give the title compound (420 mg, 0.769 mmol, 81% yield).

LCMS (ESI): Method 4, t_{R}=1.37 min, m/z (M+1)=383.1

The following compounds were prepared analogously to what described for Intermediate 13, starting from suitable intermediates (see Table 25).

TABLE 25

| | | Product Amount |
|---|---|---|
| | Structure | (Yield) |
| N° | Analytical Data | |
| Intermediate 74 | 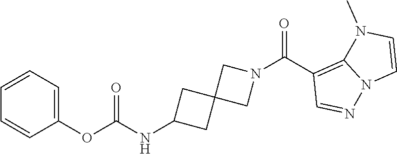 [SMILES: Cn1ccn2ncc(C(=O)N3CC4(CC(NC(=O)Oc5ccccc5)C4)C3)c12] | 625 mg (85%) |
| | LCMS (ESI): Method 1, t_{R} = 1.38 min, m/z (M + 1) = 380.2 | |
| Intermediate 75 | 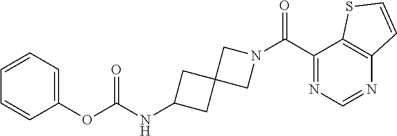 [SMILES: O=C(NC1CC2(C1)CN(C(=O)c1ncnc3ccsc13)C2)Oc1ccccc1] | 287 mg (quantitative) |

LCMS (ESI): Method 4, t_{R} = 1.53 min, m/z (M + 1) = 395.1

### Step 2—1-(2-(pyrazolo[5,1-b]thiazole-7-carbonyl)-2-azaspiro[3.3]heptan-6-yl)-3-(3-(trifluoromethoxy)phenyl)urea (Example 21)

A solution of 3-(Trifluoromethoxy) aniline (0.035 mL, 0.261 mmol) and Intermediate 13 (100 mg, 0.261 mmol) in pyridine (1.00 mL) was stirred for 16 h at 120° C. The reaction mixture was concentrated in vacuo. The residue was loaded onto an Isolute SCX-II cartridge and released with MeOH. The MeOH eluent was concentrated in vacuo and the residue purified by preparative HPLC (Sunfire C18 19×150 mm, 10 μm 5-60% ACN/H₂O (10 mM NH₄CO₃), 20 mL/minute) to give the title compound (5.7 mg, 0.0122 mmol, 5% yield).

LCMS (ESI): Method 2, t_{R}=4.24 min, m/z (M+1)=466.2

¹H NMR (400 MHz, DMSO-d6) δ 8.76 (s, 1H), 8.31 (d, J=4.3 Hz, 1H), 8.07 (s, 1H), 7.63 (s, 1H), 7.52 (dd, J=1.3, 4.0 Hz, 1H), 7.32 (t, J=8.2 Hz, 1H), 7.21 (dd, J=1.6, 7.7 Hz, 1H), 6.87-6.84 (m, 1H), 6.61 (d, J=7.6 Hz, 1H), 4.55-4.34 (m, 1H), 4.10-4.00 (m, 3H), 2.58-2.53 (m, 2H), 2.17-2.10 (m, 2H).

The following Examples and Intermediates were prepared analogously to what described for Example 21, step 2, by starting from the corresponding aniline/amine and suitable Intermediates (Table 26).

TABLE 26

| | | Product Amount |
|---|---|---|
| | Structure | (Yield) |
| N° | Analytical Data | |
| Example 22 | 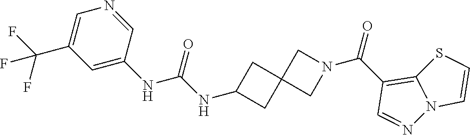 [SMILES: O=C(Nc1cncc(C(F)(F)F)c1)NC1CC2(C1)CN(C(=O)c1cnn3ccsc13)C2] | 5.7 mg, (5%) |
| | LCMS (ESI): Method 2, t_{R} = 3.63 min, m/z (M + 1) = 451.3 ¹H NMR (400 MHz, DMSO-d6) δ 9.02 (s, 1H), 8.71 (d, J = 2.1 Hz, 1H), 8.48 (d, J = 1.0 Hz, 1H), 8.37-8.31 (m, 2H), 8.08 (s, 1H), 7.52 (dd, J = 1.4, 4.1 Hz, 1H), 6.89-6.84 (m, 1H), 4.54-4.38 (m, 2H), 4.11-3.98 (m, 3H), 2.59-2.52 (m, 2H), 2.21-2.14 (m, 2H). | |
| Example 23 | 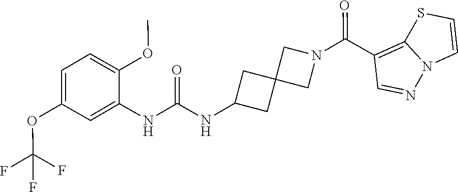 [SMILES: COc1ccc(OC(F)(F)F)cc1NC(=O)NC1CC2(C1)CN(C(=O)c1cnn3ccsc13)C2] | 7.4 mg, (6%) |
| | LCMS (ESI): Method 2, t_{R} = 4.43 min, m/z (M + 1) = 496. ¹H NMR (400 MHz, DMSO-d6) δ 8.31 (d, J = 4.4 Hz, 1H), 8.16 (d, J = 2.8 Hz, 1H), 8.09-8.06 (m, 2H), 7.52 (dd, J = 1.6, 4.0 Hz, 1H), 7.29 (d, J = 7.2 Hz, 1H), 7.03 (d, J = 8.5 Hz, 1H), 6.86-6.82 (m, 1H), 4.55-4.35 (m, 2H), 4.12-3.95 (m, 3H), 2.60- 2.54 (m, 2H), 2.09-2.02 (m, 2H). | |
| Example 24 | 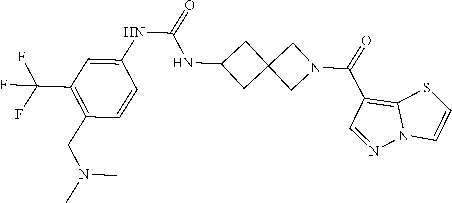 [SMILES: CN(C)Cc1ccc(NC(=O)NC2CC3(C2)CN(C(=O)c2cnn4ccsc24)C3)cc1C(F)(F)F] | 5.2 mg, (4%) |
| | LCMS (ESI): Method 3, t_{R} = 4.09 min, m/z (M + 1) = 507.2 ¹H NMR (400 MHz, DMSO-d6) δ 8.70 (s, 1H), 8.31 (d, J = 4.0 Hz, 1H), 8.07 (s, 1H), 7.87 (d, J = 2.0 Hz, 1H), 7.56-7.51 (m, 3H), 6.57-6.52 (m, 1H), 4.51-4.38 (m, 2H), 4.10-3.99 (m, 3H), 3.42 (s, 2H), 2.58-2.52 (m, 2H), 2.15-2.14 (m, 8H). | |
| Example 35 | 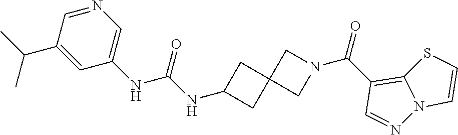 [SMILES: CC(C)c1cncc(NC(=O)NC2CC3(C2)CN(C(=O)c2cnn4ccsc24)C3)c1] | 13 mg (13%) |
| | LCMS (ESI): Method 2, t_{R} = 2.66 min, m/z (M + 1) = 425.2 ¹H NMR (400 MHz, DMSO-d6) δ 8.58 (s, 1H), 8.34 (d, J = 2.5 Hz, 1H), 8.31 (d, J = 4.0 Hz, 1H), 8.07 (s, 1H), 8.03 (d, J = 2.0 Hz, 1H), 7.76 (t, J = 2.0 Hz, 1H), 7.52 (dd, J = 1.4, 4.2 Hz, 1H), 6.64 (d, J = 7.3 Hz, 1H), 4.51-4.39 (m, 2H), 4.10-3.97 (m, 3H), 2.92-2.84 (m, 1H), 2.17-2.10 (m, 2H), 1.20 (d, J = 7.1 Hz, 6H). | |
| Example 36 | 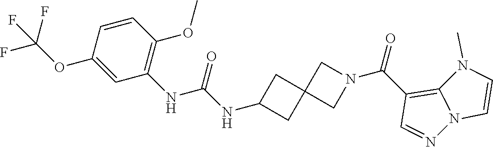 [SMILES: COc1ccc(OC(F)(F)F)cc1NC(=O)NC1CC2(C1)CN(C(=O)c1cnn3ccn(C)c13)C2] | 41 mg (31%) |
| | LCMS (ESI): Method 3, t_{R} = 4.25 min, m/z (M + 1) = 493.2 ¹H NMR (400 MHz, DMSO-d6) δ 8.17-8.15 (m, 1H), 8.05 (s, 1H), 7.77 (d, J = 1.3 Hz, 1H), 7.66 (d, J = 2.3 Hz, 1H), 7.29-7.24 (m, 2H), 7.02 (d, J = 9.0 Hz, 1H), 6.86- 6.82 (m, 1H), 4.17-3.86 (m, 11H), 2.58-2.52 (m, 2H), 2.07-2.01 (m, 2H). | |
| Example 37 | 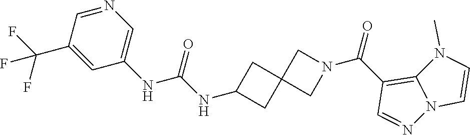 [SMILES: Cn1ccn2ncc(C(=O)N3CC4(CC(NC(=O)Nc5cncc(C(F)(F)F)c5)C4)C3)c12] | 4 mg (12%) |
| | LCMS (ESI): Method 2, t_{R} = 3.47 min, m/z (M + 1) = 448.2 ¹H NMR (400 MHz, DMSO-d6) δ 9.02 (s, 1H), 8.71 (d, J = 2.3 Hz, 1H), 8.48-8.46 (m, 1H), 8.37-8.35 (m, 1H), 7.77 (d, J = 1.0 Hz, 1H), 7.66 (d, J = 2.3 Hz, 1H), 7.26- 7.24 (m, 1H), 6.87-6.83 (m, 1H), 4.16-3.96 (m, 8H), 2.19-2.12 (m, 2H). | |
| Example 38 | 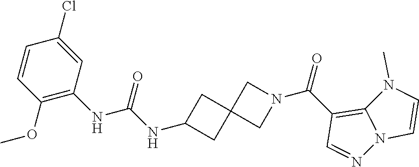 [SMILES: COc1ccc(Cl)cc1NC(=O)NC1CC2(C1)CN(C(=O)c1cnn3ccn(C)c13)C2] | 36 mg (30%) |
| | LCMS (ESI): Method 2, t_{R} = 4.01 min, m/z (M + 1) = 443.2 ¹H NMR (400 MHz, DMSO-d6) δ 8.16 (d, J = 2.5 Hz, 1H), 7.97 (s, 1H), 7.77 (d, J = 1.0 Hz, 1H), 7.66 (d, J = 2.0 Hz, 1H), 7.26-7.22 (m, 2H), 6.97 (d, J = 8.6 Hz, 1H), 6.89 (dd, J = 2.6, 8.5 Hz, 1H), 4.18-3.83 (m, 11H), 2.58-2.52 (m, 2H), 2.08-2.00 (m, 2H). | |
| Example 39 | 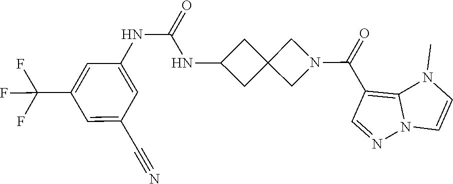 [SMILES: Cn1ccn2ncc(C(=O)N3CC4(CC(NC(=O)Nc5cc(C#N)cc(C(F)(F)F)c5)C4)C3)c12] | 15 mg (12%) |
| | LCMS (ESI): Method 2, t_{R} = 4.11 min, m/z (M + 1) = 472.2 ¹H NMR (400 MHz, DMSO-d6) δ 9.09 (s, 1H), 8.14 (s, 1H), 8.03 (s, 1H), 7.80- 7.76 (m, 2H), 7.66 (d, J = 2.3 Hz, 1H), 7.26-7.24 (m, 1H), 6.88 (d, J = 7.6 Hz, 1H), 4.19-3.96 (m, 8H), 2.57-2.52 (m, 2H), 2.20-2.12 (m, 2H). | |
| Example 41 | 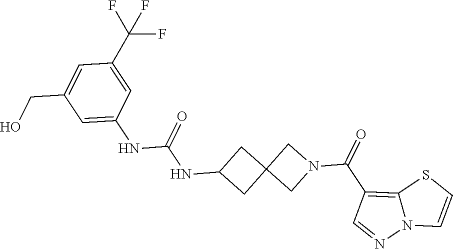 [SMILES: O=C(Nc1cc(CO)cc(C(F)(F)F)c1)NC1CC2(C1)CN(C(=O)c1cnn3ccsc13)C2] | 1.4 mg (1.2%) |
| | LCMS (ESI): Method 2, t_{R} = 3.65 min, m/z (M + 1) = 480.3 ¹H NMR (400 MHz, DMSO-d6) δ 8.83 (s, 1H), 8.31 (d, J = 4.1 Hz, 1H), 8.08 (s, 1H), 7.82 (s, 1H), 7.52 (dd, J = 1.4, 4.1 Hz, 1H), 7.46 (s, 1H), 7.16 (s, 1H), 6.64 (d, J = 7.1 Hz, 1H), 5.40-5.31 (m, 1H), 4.50-4.30 (m, 4H), 4.21-3.91 (m, 3H), 2.19- 2.12 (m, 2H). | |
| Example 42 | 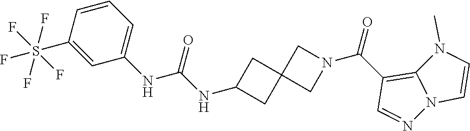 [SMILES: Cn1ccn2ncc(C(=O)N3CC4(CC(NC(=O)Nc5cccc(S(F)(F)(F)(F)F)c5)C4)C3)c12] | 1.4 mg (1.2%) |
| | LCMS (ESI): Method 2, t_{R} = 4.28 min, m/z (M + 1) = 505.2 ¹H NMR (400 MHz, DMSO-d6) δ 8.95 (s, 1H), 8.18 (t, J = 2.0 Hz, 1H), 7.77 (d, J = 0.8 Hz, 1H), 7.66 (d, J = 2.3 Hz, 1H), 7.51-7.36 (m, 3H), 7.26-7.24 (m, 1H), 6.72-6.69 (m, 1H), 4.20-3.96 (m, 8H), 2.17-2.10 (m, 2H). | |
| Example 45 | 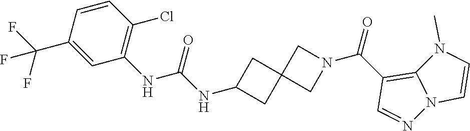 [SMILES: Cn1ccn2ncc(C(=O)N3CC4(CC(NC(=O)Nc5cc(C(F)(F)F)ccc5Cl)C4)C3)c12] | 14 mg (11%) |
| | LCMS (ESI): Method 3, t_{R} = 4.32 min, m/z (M + 1) = 481.2 ¹H NMR (400 MHz, DMSO-d6) δ 8.60 (d, J = 1.7 Hz, 1H), 8.24 (s, 1H), 7.77 (s, 1H), 7.67-7.64 (m, 2H), 7.47 (d, J = 7.2 Hz, 1H), 7.31-7.25 (m, 2H), 4.20-3.96 (m, 8H), 2.60-2.53 (m, 2H), 2.13-2.05 (m, 2H). | |
| Example 51 | 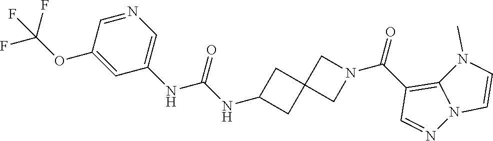 [SMILES: Cn1ccn2ncc(C(=O)N3CC4(CC(NC(=O)Nc5cncc(OC(F)(F)F)c5)C4)C3)c12] | 12 mg (10%) |
| | LCMS (ESI): Method 2, t_{R} = 3.48 min, m/z (M + 1) = 464.2 ¹H NMR (400 MHz, DMSO-d6) δ 8.99 (s, 1H), 8.46 (d, J = 2.0 Hz, 1H), 8.18-8.17 (m, 1H), 8.12-8.10 (m, 1H), 7.77 (d, J = 1.3 Hz, 1H), 7.66 (d, J = 2.3 Hz, 1H), 7.26- 7.24 (m, 1H), 6.79 (d, J = 7.8 Hz, 1H), 4.20-3.96 (m, 8H), 2.17-2.08 (m, 2H). | |
| Example 56 | 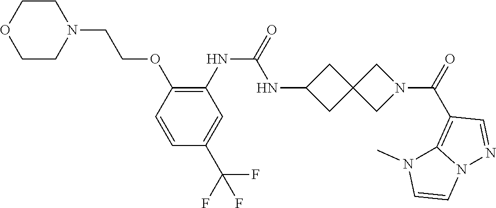 [SMILES: Cn1ccn2ncc(C(=O)N3CC4(CC(NC(=O)Nc5cc(C(F)(F)F)ccc5OCCN5CCOCC5)C4)C3)c12] | 14 mg (9%) |
| | LCMS (ESI): Method 3, t_{R} = 4.1 min, m/z (M + 1) = 576.2 ¹H NMR (400 MHz, DMSO-d6) δ 8.48 (d, J = 1.8 Hz, 1H), 7.87 (s, 1H), 7.77 (d, J = 1.0 Hz, 1H), 7.66 (d, J = 2.0 Hz, 1H), 7.39 (d, J = 7.3 Hz, 1H), 7.26-7.17 (m, 3H), 4.28-3.96 (m, 10H), 3.57 (t, J = 4.5 Hz, 4H), 2.78 (t, J = 6.1 Hz, 2H), 2.59-2.52 (m, 2H), 2.12-2.04 (m, 2H). | |
| Example 112 | 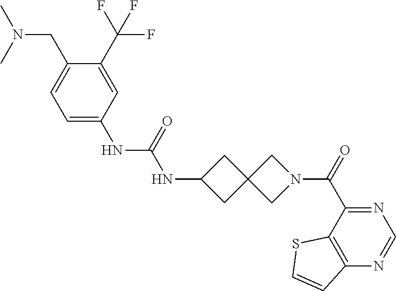 [SMILES: CN(C)Cc1ccc(NC(=O)NC2CC3(C2)CN(C(=O)c2ncnc4ccsc24)C3)cc1C(F)(F)F] | 15 mg (8%) |
| | LCMS (ESI): Method 2, t_{R} = 2.95 min, m/z (M + 1) = 519.2 ¹H NMR (400 MHz, DMSO-d6) δ 9.29 (d, J = 8.1 Hz, 1H), 8.72 (s, 1H), 8.60 (d, J = 5.6 Hz, 1H), 7.89 (br s, 1H), 7.69 (dd, J = 2.0, 5.5 Hz, 1H), 7.58-7.49 (m, 2H), 6.56 (d, J = 7.5 Hz, 1H), 4.81 (d, J = 42.1 Hz, 2H), 4.20 (d, J = 42.1 Hz, 2H), 4.05 (hept, J = 8.0 Hz, 1H), 3.42 (s, 2H), 2.64-2.53 (m, 2H), 2.21-2.11 (m, 2H), 2.15 (s, 6H). | |
| Intermediate 143 | 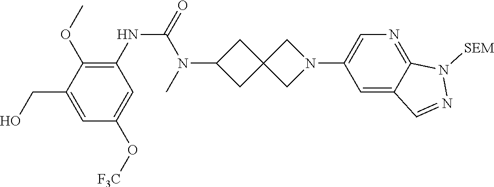 [SMILES: COc1c(CO)cc(OC(F)(F)F)cc1NC(=O)N(C)C1CC2(C1)CN(c1cnc3c(cnn3COCC[Si](C)(C)C)c1)C2] | 54 mg (55%) |
| | LCMS (ESI): Method 14, t_{R} = 4.19 min, m/z (M + 1) = 637.10 | |
| Intermediate 144 | 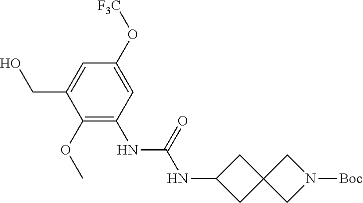 [SMILES: COc1c(CO)cc(OC(F)(F)F)cc1NC(=O)NC1CC2(C1)CN(C(=O)OC(C)(C)C)C2] | 309 mg (quantitative) |

LCMS (ESI): Method 14, t_{R} = 3.24 min, m/z (M + 1) = 476.05

### Example 25: 1-methyl-1-(2-(pyrazolo[1,5-a]pyrazine-3-carbonyl)-2-azaspiro[3.3]heptan-6-yl)-3-(3-(trifluoromethyl)phenyl)urea

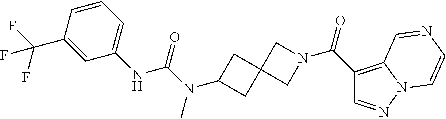 [SMILES: CN(C(=O)Nc1cccc(C(F)(F)F)c1)C1CC2(C1)CN(C(=O)c1cnn3ccncc13)C2]

### Example 25

### Step 1—tert-butyl 6-(1-methyl-3-(3-(trifluoromethyl)phenyl) ureido)-2-azaspiro[3.3]heptane-2-carboxylate (Intermediate 14)

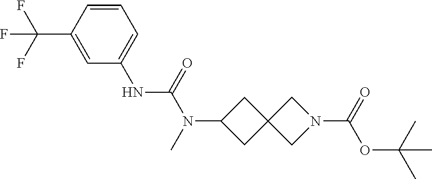 [SMILES: CN(C(=O)Nc1cccc(C(F)(F)F)c1)C1CC2(C1)CN(C(=O)OC(C)(C)C)C2]

Intermediate 14 was prepared by following the same procedure reported for the synthesis of Intermediate 6.

SM: tert-butyl 6-(methylamino)-2-azaspiro[3.3]heptane-2-carboxylate: 300 mg (1 eq);

1-isocyanato-3-(trifluoromethyl)benzene (1 eq)

Amount/yield: 478 mg, 1.16 mmol, 87%

LCMS (ESI): Method 4, t_{R}=1.76 min, m/z (M+1)=414.1

### Step 2—1-methyl-1-(2-azaspiro[3.3]heptan-6-yl)-3-(3-(trifluoromethyl)phenyl)urea (Intermediate 15, compound IVa)

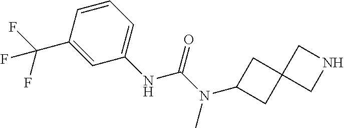 [SMILES: CN(C(=O)Nc1cccc(C(F)(F)F)c1)C1CC2(CNC2)C1]

Intermediate 15 was prepared by following the procedure described for the synthesis of Intermediate 7.

SM: Intermediate 14: 467 mg (1 eq); TFA: 0.99 mL (10 eq)

Amount/yield: 314 mg, 1.20 mmol, 93%

LCMS (ESI): Method 4, t_{R}=1.40 min, m/z (M+1)=314.1

¹H NMR (400 MHz, DMSO-d6) δ 8.58-8.55 (m, 1H), 7.88 (s, 1H), 7.72-7.68 (m, 1H), 7.45 (t, J=8.0 Hz, 1H), 7.27-7.24 (m, 1H), 4.52-3.96 (m, 5H), 2.84 (s, 3H), 2.34-2.26 (m, 2H), 2.23-2.14 (m, 2H).

### Step 3—1-methyl-1-(2-(pyrazolo[1,5-a]pyrazine-3-carbonyl)-2-azaspiro[3.3]heptan-6-yl)-3-(3-(trifluoromethyl)phenyl)urea (Example 25)

Example 25 was prepared by following the procedure described for the synthesis of Example 1, step 3.

SM: Intermediate 15: 100 mg (1 eq); pyrazolo[1,5-a]pyrazine-3-carboxylic acid: 52 mg mL (1 eq)

Amount/yield: 36 mg, 0.0790 mmol, 25%

LCMS (ESI): Method 4, t_{R}=4.11 min, m/z (M+1)=459.6

¹H NMR (400 MHz, DMSO-d6) δ 9.55 (d, J=1.5 Hz, 1H), 8.91 (dd, J=1.3, 4.8 Hz, 1H), 8.60 (s, 1H), 8.45-8.44 (m, 1H), 8.11 (d, J=4.8 Hz, 1H), 7.91 (s, 1H), 7.77-7.74 (m, 1H), 7.46 (t, J=8.0 Hz, 1H), 7.28-7.25 (m, 1H), 4.63-4.47 (m, 3H), 4.21-4.02 (m, 2H), 2.90 (s, 3H), 2.47-2.38 (m, 4H).

The following Examples and Intermediates were prepared analogously to what described for Example 25, step 3, by starting from corresponding Intermediate and the suitable carboxylic acid (see Table 27).

TABLE 27

| | | Product Amount |
|---|---|---|
| | Structure | (Yield) |
| N° | Analytical Data | |
| Example 26 | 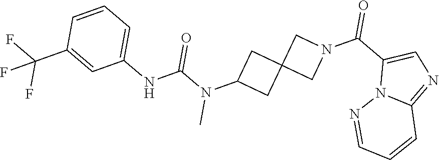 [SMILES: CN(C(=O)Nc1cccc(C(F)(F)F)c1)C1CC2(C1)CN(C(=O)c1cnc3cccnn13)C2] | 13 mg (9%) |
| | LCMS (ESI): Method 3 t_{R} = 4.35 min, m/z (M + 1) = 458.2 ¹H NMR (400 MHz, DMSO-d6) δ 9.45 (d, J = 7.1 Hz, 1H), 8.60 (s, 1H), 8.06 (s, 1H), 7.91 (s, 1H), 7.77-7.72 (m, 2H), 7.51-7.44 (m, 2H), 7.28-7.25 (m, 1H), 7.16-7.11 (m, 1H), 4.64-4.04 (m, 1H), 2.90 (s, 3H), 2.47-2.40 (m, 4H) | |
| Example 50 | 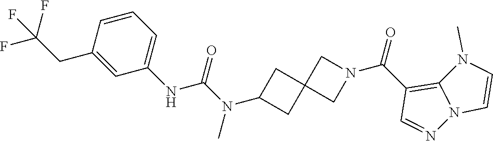 [SMILES: CN(C(=O)Nc1cccc(CC(F)(F)F)c1)C1CC2(C1)CN(C(=O)c1cnn3ccn(C)c13)C2] | 19 mg (21%) |
| | LCMS (ESI): Method 3, t_{R} = 4.1 min, m/z (M + 1) = 475.2 ¹H NMR (400 MHz, DMSO-d6) δ 8.32 (s, 1H), 7.77 (d, J = 1.0 Hz, 1H), 7.66 (d, J = 2.3 Hz, 1H), 7.48-7.40 (m, 2H), 7.26-7.20 (m, 2H), 6.92 (d, J = 7.6 Hz, 1H), 4.60-3.96 (m, 8H), 3.55 (q, J = 11.6 Hz, 2H), 2.87 (s, 3H), 2.44-2.31 (m, 4H). | |
| Example 64 | 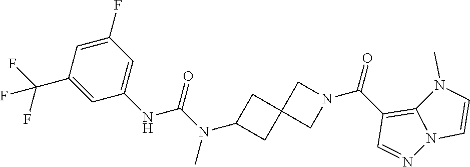 [SMILES: CN(C(=O)Nc1cc(F)cc(C(F)(F)F)c1)C1CC2(C1)CN(C(=O)c1cnn3ccn(C)c13)C2] | 16 mg (13%) |
| | LCMS (ESI): Method 3, t_{R} = 4.33 min, m/z (M + 1) = 479.2 ¹H NMR (400 MHz, DMSO-d6) δ 8.79 (s, 1H), 7.77-7.71 (m, 3H), 7.66 (d, J = 2.0 Hz, 1H), 7.26 (dd, J = 1.3, 2.3 Hz, 1H), 7.16 (d, J = 8.6 Hz, 1H), 4.59- 4.49 (m, 1H), 4.43-4.04 (m, 4H), 3.96 (s, 3H), 2.89 (s, 3H), 2.42 2.37 (m, 4H). | |
| Example 81 | 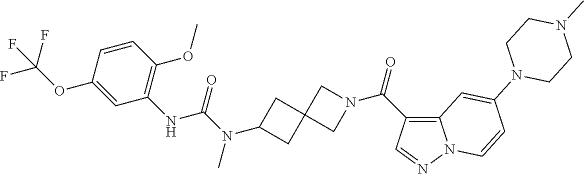 [SMILES: COc1ccc(OC(F)(F)F)cc1NC(=O)N(C)C1CC2(C1)CN(C(=O)c1cnn3ccc(N4CCN(C)CC4)cc13)C2] | 25 mg (26%) |
| | LCMS (ESI): Method 3, t_{R} = 3.63 min, m/z (M + 1) = 602.5 ¹H NMR (400 MHz, CDCl₃) δ 8.25 (d, J = 7.8 Hz, 1H), 8.21-8.21 (m, 1H), 7.91 (s, 1H), 7.58 (d, J = 2.6 Hz, 1H), 7.09 (s, 1H), 6.81 (s, 2H), 6.67 (dd, J = 2.9, 7.8 Hz, 1H), 4.56 (d, J = 8.0 Hz, 1H), 4.37-4.37 (m, 4H), 3.90 (s, 3H), 3.37 (t, J = 5.0 Hz, 4H), 2.95 (s, 3H), 2.64-2.54 (m, 6H), 2.42-2.37 (m, 2H), 2.36 (s, 3H). | |
| Example 133 | 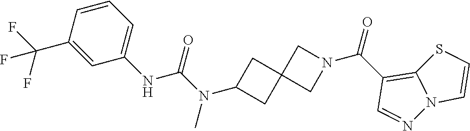 [SMILES: CN(C(=O)Nc1cccc(C(F)(F)F)c1)C1CC2(C1)CN(C(=O)c1cnn3ccsc13)C2] | 30 mg (22%) |
| | LCMS (ESI): Method 3, t_{R} = 4.25 min, m/z (M + 1) = 464.2 ¹H NMR (400 MHz, DMSO-d6) δ 8.60 (s, 1H), 8.31 (d, J = 4.0 Hz, 1H), 8.07 (s, 1H), 7.91 (s, 1H), 7.75 (d, J = 8.3 Hz, 1H), 7.53 (dd, J = 1.4, 4.2 Hz, 1H), 7.46 (t, J = 8.1 Hz, 1H), 7.26 (d, J = 7.6 Hz, 1H), 4.61-4.41 (m, 3H), 4.12- 3.95 (m, 2H), 2.90 (s, 3H), 2.48-2.37 (m, 4H). | |
| Example 134 | 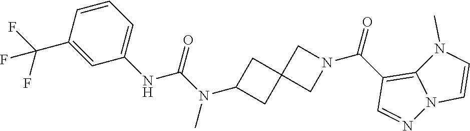 [SMILES: CN(C(=O)Nc1cccc(C(F)(F)F)c1)C1CC2(C1)CN(C(=O)c1cnn3ccn(C)c13)C2] | 24 mg (18%) |
| | LCMS (ESI): Method 3, t_{R} = 4.1 min, m/z (M + 1) = 461.2 ¹H NMR (400 MHz, DMSO-d6) δ 8.59 (s, 1H), 7.91 (s, 1H), 7.78-7.73 (m, 2H), 7.66 (d, J = 2.3 Hz, 1H), 7.46 (t, J = 8.0 Hz, 1H), 7.28-7.24 (m, 2H), 4.60- 3.97 (m, 8H), 2.89 (s, 3H), 2.45-2.31 (m, 4H). | |
| Intermediate 76 | 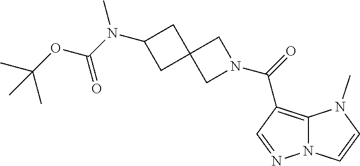 [SMILES: CN(C(=O)OC(C)(C)C)C1CC2(C1)CN(C(=O)c1cnn3ccn(C)c13)C2] | 370 mg (76%) |

LCMS (ESI): Method 1, t_{R} = 1.49 min, m/z (M + 1) = 374.2

### Intermediate 77: 1-(2-azaspiro[3.3]heptan-6-yl)-1-(2,2,2-trifluoroethyl)-3-(3-(trifluoromethyl)phenyl)urea

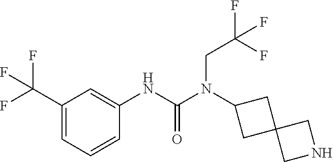 [SMILES: O=C(Nc1cccc(C(F)(F)F)c1)N(CC(F)(F)F)C1CC2(CNC2)C1]

### Step 1-tert-butyl 6-((2,2,2-trifluoroethyl)amino)-2-azaspiro[3.3]heptane-2-carboxylate (Intermediate 78)

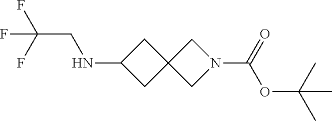 [SMILES: CC(C)(C)OC(=O)N1CC2(CC(NCC(F)(F)F)C2)C1]

A solution of 2,2,2-trifluoroethan-1-amine (0.14 mg, 1.42 mmol, 1.20 eq) and tert-butyl 6-oxo-2-azaspiro[3.3]heptane-2-carboxylate (250 mg, 1.18 mmol) in MeOH (1.5 mL) was stirred at rt for 18 h. The reaction cooled to 0° C. and NaBH₄ (0.067 mg, 1.8 mmol) was added and stirring continued at rt for 3 h. The reaction mixture was concentrated in vacuo and the residue partitioned between DCM and saturated Na₂CO₃. The organic extract was filtered through a hydrophobic frit and concentrated in vacuo. The title compound was used without further purification (350 mg, 1.18 mmol, quantitative).

LCMS (ESI): Method 4, t_{R}=1.57 min, m/z (M+1-tBu)=239.2

The following Intermediate was prepared analogously to what described for Intermediate 78, by starting from corresponding Intermediate (see Table 28).

TABLE 28

| | | Product |
|---|---|---|
| | | Amount |
| | Structure | (Yield) |
| N° | Analytical Data | |
| Intermediate 79 | 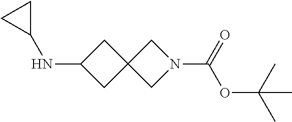 [SMILES: CC(C)(C)OC(=O)N1CC2(CC(NC3CC3)C2)C1] | 298 mg (quantitative) |

LCMS (ESI): Method 4, t_{R} = 1.55 min, m/z (M + 1) = 253.3

### Step 2—tert-butyl 6-(1-(2,2,2-trifluoroethyl)-3-(3-(trifluoromethyl)phenyl) ureido)-2-azaspiro[3.3]heptane-2-carboxylate (Intermediate 80)

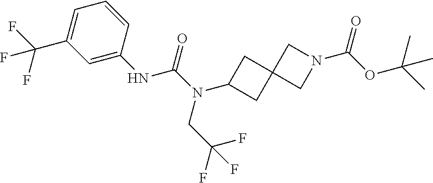 [SMILES: CC(C)(C)OC(=O)N1CC2(CC(N(CC(F)(F)F)C(=O)Nc3cccc(C(F)(F)F)c3)C2)C1]

Intermediate 80 was prepared by following the same procedure reported for the synthesis of Intermediate 6.

SM: Intermediate 78: 350 mg (1 eq); 1-isocyanato-3-(trifluoromethyl)benzene: 0.163 mL (1 eq)

Amount/yield: 127 mg, 22%

LCMS (ESI): Method 4, t_{R}=1.85 min, m/z (M+1-tBu)=426.2

The following compound was prepared analogously to what described for Intermediate 80, by starting from suitable Intermediate (see Table 29).

TABLE 29

| | | Product Amount |
|---|---|---|
| Intermediate | Structure | (Yield) |
| N° | Analytical Data | |
| Intermediate 81 | 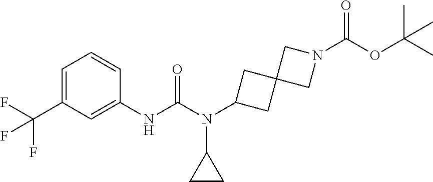 [SMILES: CC(C)(C)OC(=O)N1CC2(CC(N(C(=O)Nc3cccc(C(F)(F)F)c3)C3CC3)C2)C1] | 518 mg (quantitative) |

LCMS (ESI): Method 4, t_{R} = 1.84 min, m/z (M + 1-tBu) = 384.1

### Step 3—1-(2-azaspiro[3.3]heptan-6-yl)-1-(2,2,2-trifluoroethyl)-3-(3-(trifluoromethyl)phenyl) urea (Intermediate 77)

Intermediate 77 was prepared by following the same procedure reported for the synthesis of Intermediate 7.

SM: Intermediate 80: 127 mg (1 eq)

Amount/yield: 100 mg, quantitative

LCMS (ESI): Method 4, t_{R}=1.57 min, m/z (M+1)=382.1

The following compound was prepared analogously to what described for Intermediate 77, by starting from suitable Intermediates (see Table 30).

TABLE 30

| | | Product Amount |
|---|---|---|
| | Structure | (Yield) |
| N° | Analytical Data | |
| Intermediate 82 | 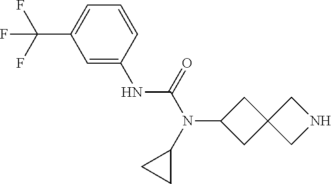 [SMILES: O=C(Nc1cccc(C(F)(F)F)c1)N(C1CC1)C1CC2(CNC2)C1] | 401 mg (quantitative) |

LCMS (ESI): Method 4, t_{R} = 1.50 min, m/z (M + 1) = 340.1

### Example 27: 2-(2-(pyrazolo[5,1-b]thiazole-7-carbonyl)-2-azaspiro[3.3]heptan-6-yl)-N-(3-(trifluoromethyl)phenyl)acetamide

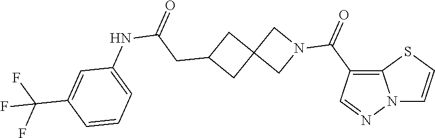 [SMILES: O=C(CC1CC2(C1)CN(C(=O)c1cnn3ccsc13)C2)Nc1cccc(C(F)(F)F)c1]

### Example 27

### Step 1—tert-butyl 6-(2-oxo-2-((3-(trifluoromethyl)phenyl)amino)ethyl)-2-azaspiro[3.3]heptane-2-carboxylate (Intermediate 16)

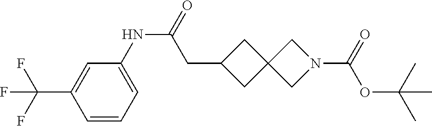 [SMILES: CC(C)(C)OC(=O)N1CC2(CC(CC(=O)Nc3cccc(C(F)(F)F)c3)C2)C1]

Intermediate 16 was prepared by following the procedure described for the synthesis of Example 1, step 3.

SM: 2-(2-tert-butoxycarbonyl-2-azaspiro[3.3]heptan-6-yl) acetic acid: 300 mg (1 eq.); 3-(trifluoromethyl) aniline: 0.15 mL, (1 eq.)

Amount/yield: 430 mg, 1.08 mmol, 92%

LC-MS (ESI): Method 1, t_{R}=1.75 min, m/z (M+H-tBu)=343.0

The following compounds were prepared analogously to what described for Intermediate 16, by starting from suitable Intermediates (see Table 31).

TABLE 31

| | | Product Amount |
|---|---|---|
| | Structure | (Yield) |
| N° | Analytical Data | |
| Intermediate 145 | 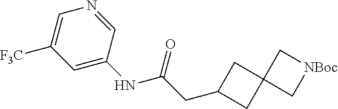 [SMILES: CC(C)(C)OC(=O)N1CC2(CC(CC(=O)Nc3cncc(C(F)(F)F)c3)C2)C1] | 120 mg (38%) |
| | LCMS (ESI): Method 1, t_{R} = 1.73 min, m/z (M + 1) = 398.2 | |
| Intermediate 146 | 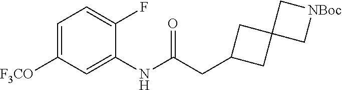 [SMILES: CC(C)(C)OC(=O)N1CC2(CC(CC(=O)Nc3cc(OC(F)(F)F)ccc3F)C2)C1] | 80 mg (24%) |
| | LCMS (ESI): Method 1, t_{R} = 1.86 min, m/z (M + 1) = 431.3 | |
| Intermediate 147 | 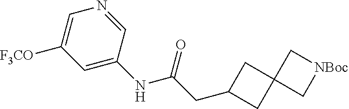 [SMILES: CC(C)(C)OC(=O)N1CC2(CC(CC(=O)Nc3cncc(OC(F)(F)F)c3)C2)C1] | 95 mg (29%) |

LCMS (ESI): Method 1, t_{R} = 1.76 min, m/z (M + 1) = 414.2

### Step 2—2-(2-azaspiro[3.3]heptan-6-yl)-N-[3-(trifluoromethyl)phenyl]acetamide (Intermediate 17, compound IVb)

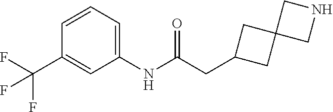 [SMILES: O=C(CC1CC2(CNC2)C1)Nc1cccc(C(F)(F)F)c1]

Intermediate 17 was prepared by following the procedure described for the synthesis of Intermediate 7.

SM: Intermediate 16: 430 mg (1 eq.); TFA: 1.7 ml, (20 eq)

Amount/yield: 230 mg, 0.270 mmol, 25%

LC-MS (ESI): Method 1 t_{R}=1.30 min, m/z (M+1)=299.0

The following compounds were prepared analogously to what described for Intermediate 17, by starting from suitable Intermediates (see Table 32).

TABLE 32

| | | Product Amount |
|---|---|---|
| | Structure | (Yield) |
| N° | Analytical Data | |
| Intermediate 148 | 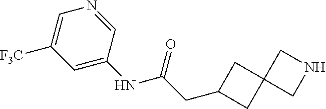 [SMILES: O=C(CC1CC2(CNC2)C1)Nc1cncc(C(F)(F)F)c1] | 80 mg (89%) |
| | LCMS (ESI): Method 1, t_{R} = 1.11 min, m/z (M + 1) = 300.0 | |
| Intermediate 149 | 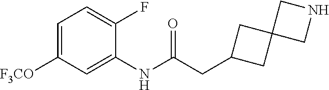 [SMILES: O=C(CC1CC2(CNC2)C1)Nc1cc(OC(F)(F)F)ccc1F] | 60 mg (82%) |
| | LCMS (ESI): Method 1, t_{R} = 1.25 min, m/z (M + 1) = 333.0 | |
| Intermediate 150 | 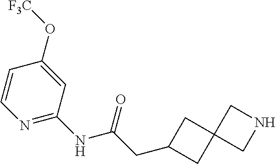 [SMILES: O=C(CC1CC2(CNC2)C1)Nc1cc(OC(F)(F)F)ccn1] | 336 mg (100%) |
| | LCMS (ESI): Method 7, t_{R} = 0.48 min, m/z (M + 1) = 316.14 | |
| Intermediate 151 | 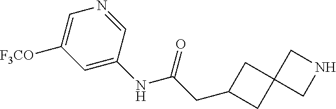 [SMILES: O=C(CC1CC2(CNC2)C1)Nc1cncc(OC(F)(F)F)c1] | 40 mg (55%) |
| | LCMS (ESI): Method 1, t_{R} = 1.14 min, m/z (M + 1) = 316.0 | |
| Intermediate 152 | 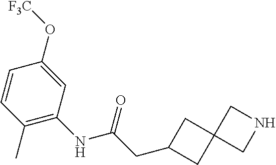 [SMILES: Cc1ccc(OC(F)(F)F)cc1NC(=O)CC1CC2(CNC2)C1] | 206 mg (100%) |
| | LCMS (ESI): Method 7, t_{R} = 0.55 min, m/z (M + 1) = 329.1 | |
| Intermediate 153 | 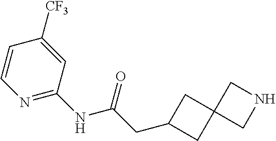 [SMILES: O=C(CC1CC2(CNC2)C1)Nc1cc(C(F)(F)F)ccn1] | 324 mg (100%) |

LCMS (ESI): Method 7, t_{R} = 0.45 min, m/z (M + 1) = 300.1

### Step 3—2-(2-(pyrazolo[5,1-b]thiazole-7-carbonyl)-2-azaspiro[3.3]heptan-6-yl)-N-(3-(trifluoromethyl)phenyl)acetamide (Example 27)

Example 27 was prepared by following the procedure described for the synthesis of Example 1, step 3.

SM: Intermediate 17: 230 mg (1 eq.); pyrazolo[5,1-b]thiazole-7-carboxylic acid: 45 mg, (1 eq.)

Amount/yield: 31 mg, 0.0694 mmol, 26%

LC-MS (ESI): Method 3 t_{R}=4.35 min, m/z (M+1)=449.2

¹H NMR (400 MHz, DMSO-d6) δ 10.20 (s, 1H), 8.31 (d, J=4.3 Hz, 1H), 8.08 (d, J=10.8 Hz, 2H), 7.76 (d, J=8.8 Hz, 1H), 7.56-7.51 (m, 2H), 7.38 (d, J=7.8 Hz, 1H), 4.49-4.37 (m, 2H), 4.06-3.96 (m, 2H), 2.61-2.52 (m, 1H), 2.46 (d, J=7.6 Hz, 2H), 2.42-2.34 (m, 2H), 2.01-1.94 (m, 2H).

The following compounds were prepared as described for Example 27, step 3, by starting from Intermediate 17 and the suitable carboxylic acid (see Table 33).

TABLE 33

| | | Product Amount |
|---|---|---|
| | Structure | (Yield) |
| N° | Analytical Data | |
| Example 28 | 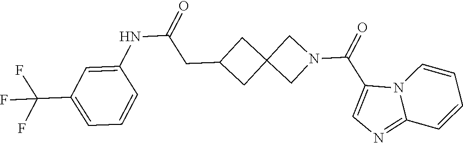 [SMILES: O=C(CC1CC2(C1)CN(C(=O)c1cnc3ccccn13)C2)Nc1cccc(C(F)(F)F)c1] | 13 mg (8%) |
| | LC-MS (ESI): Method 2 t_{R} = 3.72 min, m/z (M + 1) = 443.2 | |
| | ¹H NMR (400 MHZ, DMSO-d6) δ 10.20 (s, 1H), 9.45 (d, J = 6.8 Hz, 1H), | |
| | 8.07 (d, J = 13.9 Hz, 2H), 7.78-7.71 (m, 2H), 7.53 (t, J = 8.0 Hz, 1H), 7.50- | |
| | 7.45 (m, 1H), 7.39-7.36 (m, 1H), 7.13 (dt, J = 1.7, 6.8 Hz, 1H), 4.53-4.04 (m, 4H), | |
| | 2.62-2.52 (m, 1H), 2.48-2.44 (m, 2H), 2.43-2.35 (m, 2H), 2.02-1.95 (m, 2H). | |
| Example 29 | 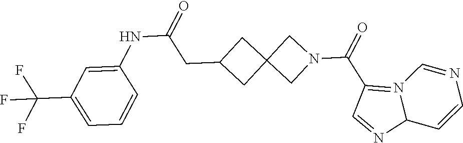 | 18 mg (11%) |

LC-MS (ESI): Method 3 t_{R} = 4.05 min, m/z (M + 1) = 444.2
¹H NMR (400 MHZ, DMSO-d6) δ 10.23-10.19 (m, 1H), 9.55-9.54 (m, 1H), 8.90
(dd, J = 1.5, 4.8 Hz, 1H), 8.45-8.41 (m, 1H), 8.11-8.09 (m, 2H), 7.78-7.74
(m, 1H), 7.53 (t, J = 8.0 Hz, 1H), 7.39-7.35 (m, 1H), 4.59-4.41 (m, 2H), 4.16-4.02
(m, 2H), 2.59 (s, 1H), 2.45 (d, J = 7.3 Hz, 2H),2.43-2.35 (m, 2H), 2.02-1.96 (m, 2H).

### Example 47: 1-methyl-1-(2-(pyrazolo[5,1-b]thiazole-7-carbonyl)-2-azaspiro[3.3]heptan-6-yl)-3-(5-(trifluoromethyl)pyridin-3-yl)urea

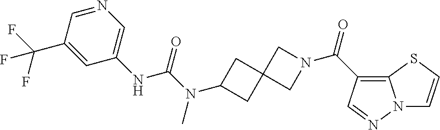 [SMILES: CN(C(=O)Nc1cncc(C(F)(F)F)c1)C1CC2(C1)CN(C(=O)c1cnn3ccsc13)C2]

### Example 47

To a stirred solution of triphosgene (258 mg, 0.87 mmol) in DCM (2 mL) at 0° C. was added a solution of 5-(trifluoromethyl)pyridin-3-amine (94 mg, 0.579 mmol, 2.00 eq) and Et₃N (0.12 mL, 0.87 mmol) in DCM (2 mL) dropwise, the reaction was then stirred at 0° C. for 1 h. Intermediate 68 (80 mg, 0.289 mmol, 1.00 eq) and Et₃N (0.16 mL, 1.15 mmol) in DCM were added dropwise, then after addition the reaction was allowed to warm to rt and the reaction was stirred overnight. The reaction mixture was diluted with DCM and washed with NaHCO₃ and brine. The organic phase was dried over MgSO₄ and concentrated in vacuo. The crude residue was purified by FCC (0-10% MeOH in DCM) to give the title compound (20.7 mg, 0.045 mmol, 15%).

LCMS (ESI): Method 3, t_{R}=3.72 min, m/z (M+1)=465.2

¹H NMR (400 MHz, DMSO-d6) δ 8.94 (d, J=2.3 Hz, 1H), 8.84 (s, 1H), 8.52 (d, J=1.0 Hz, 1H), 8.31 (dd, J=3.8, 3.8 Hz, 2H), 8.07 (s, 1H), 7.53 (dd, J=1.4, 4.2 Hz, 1H), 4.62-4.36 (m, 3H), 4.17-3.96 (m, 2H), 2.92 (s, 3H), 2.47-2.39 (m, 4H).

Analogously, the following Examples were prepared as described for Example 47 by starting from suitable Intermediate (see Table 34).

TABLE 34

| | | Product Amount |
|---|---|---|
| | Structure | (Yield) |
| N° | Analytical Data | |
| Example 48 | 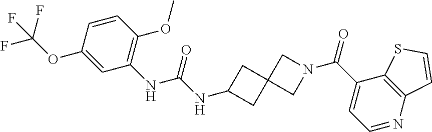 [SMILES: COc1ccc(OC(F)(F)F)cc1NC(=O)NC1CC2(C1)CN(C(=O)c1ccnc3ccsc13)C2] | 13 mg (13%) |
| | LCMS (ESI): Method 3, t_{R} = 4.56 min, m/z (M + 1) = 507.2 | |
| | ¹H NMR (400 MHZ, DMSO-d6) δ 8.78 (dd, J = 4.9, 4.9 Hz, 1H), 8.22 (d, | |
| | J = 5.6 Hz, 1H), 8.15 (s, 1H), 8.05 (d, J = 6.6 Hz, 1H), 7.60 (d, J = 5.6 Hz, 1H), | |
| | 7.56 (dd, J = 5.4, 5.4 Hz, 1H), 7.27 (dd, J = 6.9, 6.9 Hz, 1H), 7.02 (d, J = 8.8 | |
| | Hz, 1H), 6.84 (dd, J = 2.4, 8.5 Hz, 1H), 4.60 (s, 1H), 4.49 (s, 1H), 4.23 (s, | |
| | 1H), 4.13 (s, 1H), 4.06-3.94 (m, 1H), 3.86 (s, 3H), 2.62-2.55 (m, 2H), 2.11-2.01 (m, 2H). | |
| Example 49 | 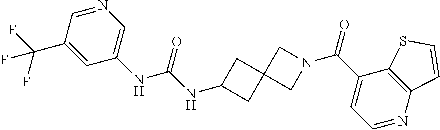 [SMILES: O=C(Nc1cncc(C(F)(F)F)c1)NC1CC2(C1)CN(C(=O)c1ccnc3ccsc13)C2] | 15 mg (10%) |
| | LCMS (ESI): Method 3, t_{R} = 3.86 min, m/z (M + 1) = 462.2 | |
| | ¹H NMR (400 MHZ, DMSO-d6) δ 9.16-9.11 (m, 1H), 8.84 (dd, J = 4.9, 4.9 | |
| | Hz, 1H), 8.76 (d, J = 2.3 Hz, 1H), 8.53 (d, J = 1.0 Hz, 1H), 8.41 (dd, J = 2.0, | |
| | 2.0 Hz, 1H), 8.27 (d, J = 5.6 Hz, 1H), 7.66 (d, J = 5.6 Hz, 1H), 7.61 (dd, J = 5.2, | |
| | 5.2 Hz, 1H), 6.96 (d, J = 7.3 Hz, 1H), 4.66 (s, 1H), 4.54 (s, 1H), 4.29 (s, 1H), | |
| | 4.18 (s, 1H), 4.16-4.04 (m, 1H), 2.66-2.59 (m, 3H), 2.27-2.19 (m, 2H). | |
| Example 58 | 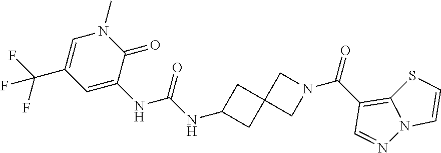 [SMILES: Cn1cc(C(F)(F)F)cc(NC(=O)NC2CC3(C2)CN(C(=O)c2cnn4ccsc24)C3)c1=O] | 16 mg (11%) |
| | LCMS (ESI): Method 2, t_{R} = 3.61 min, m/z (M + 1) = 481.2 | |
| | ¹H NMR (400 MHZ, DMSO-d6) δ 8.49 (s, 1H), 8.36 (d, J = 4.0 Hz, 1H), 8.23 | |
| | (d, J = 2.5 Hz, 1H), 8.13 (s, 1H), 7.99 (d, J = 1.3 Hz, 1H), 7.60-7.57 (m, 2H), 4.59-4.42 | |
| | (m, 2H), 4.18-4.00 (m, 3H), 3.62 (s, 3H), 2.66-2.58 (m, 2H), 2.15-2.08 (m, 2H). | |
| Example 208 | 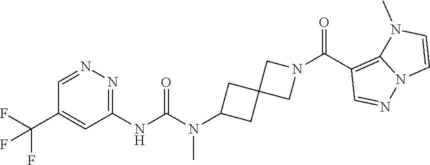 [SMILES: CN(C(=O)Nc1cc(C(F)(F)F)cnn1)C1CC2(C1)CN(C(=O)c1cnn3ccn(C)c13)C2] | 29 mg (12%) |
| | LCMS (ESI): Method 2, t_{R} = 3.45 min, m/z (M + 1) = 463.4 | |
| | ¹H NMR (400 MHZ, DMSO-d6) δ 10.20 (s, 1H), 9.26 (d, J = 1.6 Hz, 1H), | |
| | 8.33 (s, 1H), 7.76 (s, 1H), 7.65 (d, J = 2.2 Hz, 1H), 7.25 (s, 1H), 4.66-4.53 | |
| | (m, 1H), 4.53-3.86 (m, 4H), 3.96 (s, 3H), 2.94 (s, 3H), 2.47-2.34 (m, 4H). | |
| Example 267 | 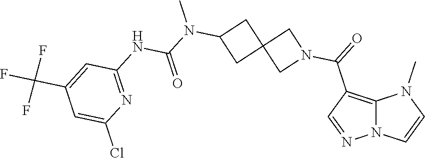 [SMILES: CN(C(=O)Nc1cc(C(F)(F)F)cc(Cl)n1)C1CC2(C1)CN(C(=O)c1cnn3ccn(C)c13)C2] | 18 mg (14%) |
| | LCMS (ESI): Method 11, t_{R} = 4.35 min, m/z (M + 1) = 495.6 | |
| | ¹H NMR (400 MHZ, DMSO-d6) δ 9.81 (s, 1H), 8.11 (s, 1H), 7.76 (s, | |
| | 1H), 7.66 (d, J = 2.2 Hz, 1H), 7.47 (s, 1H), 7.26 (dd, J = 1.2, 2.2 Hz, 1H), | |
| | 4.61-4.50 (m, 1H), 4.49-3.77 (m, 4H), 3.96 (s, 3H), 2.89 (s, 3H), 2.47-2.28 (m, 4H). | |
| Example 272 | 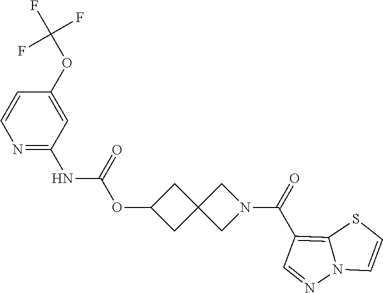 [SMILES: O=C(Nc1cc(OC(F)(F)F)ccn1)OC1CC2(C1)CN(C(=O)c1cnn3ccsc13)C2] | 7.3 mg (8%) |
| | LCMS (ESI): Method 11, t_{R} = 3.96 min, m/z (M + 1) = 468.5 | |
| | ¹H NMR (400 MHZ, CDCl₃) δ 10.63 (s, 1H), 8.38 (d, J = 5.6 Hz, 1H), 8.32 | |
| | (d, J = 4.1 Hz, 1H), 8.06 (d, J = 1.3 Hz, 1H), 7.79-7.79 (m, 1H), 7.53 (dd, | |
| | J = 1.4, 4.1 Hz, 1H), 7.08-7.06 (m, 1H), 4.95-4.87 (m, 1H), 4.57-4.39 | |
| | (m, 2H), 4.13-3.99 (m, 2H), 2.74-2.67 (m, 2H), 2.36-2.28 (m, 2H). | |
| Example 273 | 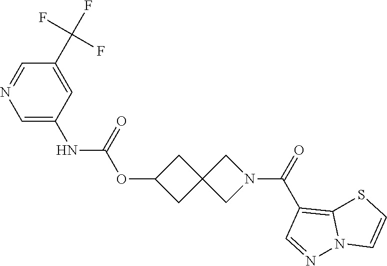 [SMILES: O=C(Nc1cncc(C(F)(F)F)c1)OC1CC2(C1)CN(C(=O)c1cnn3ccsc13)C2] | 18 mg (21%) |
| | LCMS (ESI): Method 11, t_{R} = 3.44 min, m/z (M + 1) = 452.6 | |
| | ¹H NMR (400 MHZ, DMSO-d6) δ 10.34-10.34 (m, 1H), 8.85 (d, J = 2.4 | |
| | Hz, 1H), 8.60 (s, 1H), 8.32 (d, J = 4.1 Hz, 1H), 8.27 (dd, J = 2.3, 2.3 Hz, | |
| | 1H), 8.06 (s, 1H), 7.53 (dd, J = 1.4, 4.1 Hz, 1H), 4.98-4.89 (m, 1H), 4.56- | |
| | 4.45 (m, 2H), 4.14-4.03 (m, 2H), 2.75-2.67 (m, 2H), 2.38-2.31 (m, 2H). | |
| Example 308 | 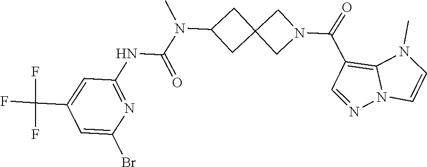 [SMILES: CN(C(=O)Nc1cc(C(F)(F)F)cc(Br)n1)C1CC2(C1)CN(C(=O)c1cnn3ccn(C)c13)C2] | 31 mg (25%) |

LCMS (ESI): Method 11, t_{R} = 4.45 min, m/z (M + 1) = 540.3, 542.2
¹H NMR (400 MHZ, DMSO-d6) δ 9.82 (s, 1H), 8.12 (s, 1H), 7.77 (s, 1H),
7.66 (d, J = 2.2 Hz, 1H), 7.58 (s, 1H), 7.26 (dd, J = 1.2, 2.2 Hz, 1H), 4.62-
4.49 (m, 1H), 4.49-3.83 (m, 4H), 3.96 (s, 3H), 2.89 (s, 3H), 2.45-2.31 (m, 4H).

### Example 146: 2-(1-methyl-1H-imidazo[1,2-b]pyrazole-7-carbonyl)-2-azaspiro[3.3]heptan-6-yl (5-(trifluoromethyl)pyridin-3-yl)carbamate

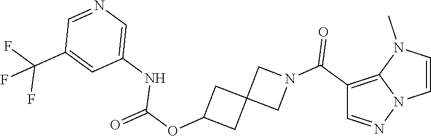 [SMILES: Cn1ccn2ncc(C(=O)N3CC4(CC(OC(=O)Nc5cncc(C(F)(F)F)c5)C4)C3)c12]

### Example 146

Intermediate 56 (120 mg, 0.46 mmol) was dissolved in DCM (2 mL) and added to 4-nitrophenyl chloroformate (112 mg, 0.553 mmol) and pyridine (0.056 mL). Solution was stirred at rt for 2 h, then solvent was removed under vacuum. The solid was dissolved in 1 mL pyridine and added of 5-(trifluoromethyl)pyridin-3-amine (90 mg, 0.55 mmol) and solution was stirred at 60° C. Then solvent was removed under vacuum. The solid was dissolved in DCM and washed with NaHCO₃ sat. solution. After purification by RF FCC, appropriate fractions were combined and concentrated, aqueous phase was quenched with NaHCO₃ sat solution and extracted with DCM and MeTHF. Organic layer was evaporated to give title product (45 mg, 0.10 mmol, 22% yield).

LCMS (ESI): Method 5, t_{R}=5.23 min, m/z (M+1)=449.30

¹H NMR (400 MHz, DMSO-d6) δ ppm 10.33 (1H, s), 8.84 (1H, d, J=2.19 Hz), 8.60 (1H, s), 8.27 (1H, br s), 7.76 (1H, d, J=1.10 Hz), 7.66 (1H, d, J=2.19 Hz), 7.26 (1H, dd, J=1.97, 1.10 Hz), 4.92 (1H, m), 4.32 (4H, m), 3.96 (3H, s), 2.70 (2H, td, J=6.96, 3.62 Hz), 2.32 (2H, m).

The following Example was prepared analogously to what described for Example 146 by starting from suitable Intermediate (see Table 35).

TABLE 35

| | | Product Amount |
|---|---|---|
| | Structure | (Yield) |
| N° | Analytical Data | |
| Example 144 | 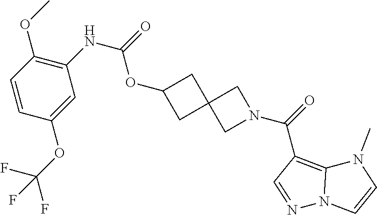 [SMILES: COc1ccc(OC(F)(F)F)cc1NC(=O)OC1CC2(C1)CN(C(=O)c1cnn3ccn(C)c13)C2] | 15 mg (8%) |

LCMS (ESI): Method 5, t_{R} = 6.87 min, m/z (M + 1) = 494.20
¹H NMR (400 MHZ, DMSO-d6) δ ppm 8.71 (1 H, s), 7.76 (1 H, d, J = 1.10
Hz), 7.75 (1 H, br s), 7.66 (1 H, d, J = 2.19 Hz), 7.26 (1 H, m), 7.09 (1 H,
m), 7.04 (1 H, m), 4.88 (1 H, t, J = 7.13 Hz), 4.18 (4 H, m), 3.96 (3 H, s),
3.83 (3 H, s), 2.66 (2 H, ddd, J = 10.14, 7.07, 2.96 Hz), 2.29 (2 H, m).

### Intermediate 154: (benzyl(2-(1-(2-methoxyethyl)-1H-imidazo[1,2-b]pyrazole-7-carbonyl)-2-azaspiro[3.3]heptan-6-yl)(methyl)carbamate)

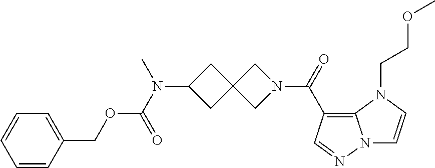 [SMILES: COCCn1ccn2ncc(C(=O)N3CC4(CC(N(C)C(=O)OCc5ccccc5)C4)C3)c12]

Intermediate 154 was prepared by following the procedure for Intermediate 30, starting from Intermediate 132 (860 mg, 3.6 mmol) and 2-bromoethyl methyl ether (0.3 ml, 5.5 mmol).

Amount/yield: 430 mg, 0.95 mmol mmol, 44%

LC-MS (ESI): Method 7, t_{R}=0.95 min, m/z (M+1)=452.25

### Intermediate 155: (1-(2-methoxyethyl)-1H-imidazo[1,2-b]pyrazol-7-yl) (6-(methylamino)-2-azaspiro[3.3]heptan-2-yl) methanone

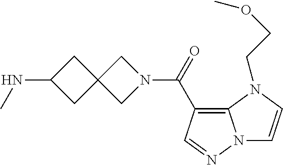 [SMILES: CNC1CC2(C1)CN(C(=O)c1cnn3ccn(CCOC)c13)C2]

In a screwable two-chamber glass system, Intermediate 154 was dissolved in ethanol (5 mL) then Pd/C (50% wet) (10.13 mg, 0.05 mmol) was splitted in both chambers. The vessel was closed and 3 cycle vacuum-N₂ was performed to set the reaction. Triethylsilane (1.52 ml, 9.5 mmol) was added slowly to chamber 1 and then the reaction was stirred at rt for 2 h. The reaction mixture was filtered on a paper filter to removed the catalyst, washing with EtOH and then the organic phase was concentrated under vacuum to afford the title product (260 mg, 0.82 mmol, 86%).

LCMS (ESI): Method 7, t_{R}=0.31 min, m/z (M+1)=318.17

The following compound was prepared according to the same synthetic procedure (see Table 36).

TABLE 36

| | | Product Amount |
|---|---|---|
| | Structure | (Yield) |
| N° | Analytical Data | |
| Intermediate 156 | 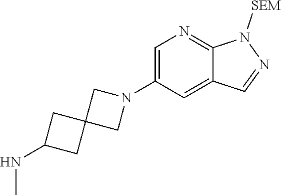 [SMILES: CNC1CC2(C1)CN(c1cnc3c(cnn3COCC[Si](C)(C)C)c1)C2] | 210 mg (quantitative) |

LCMS (ESI): Method 4 min, t_{R} = 1.17 min, m/z (M + 1) = 374.20
¹H NMR (300 MHz, DMSO-d6) δ 7.99 (d, J = 1.4 Hz, 1H), 7.96 (dd,
J = 2.6, 1.4 Hz, 1H), 7.10 (t, J = 2.4 Hz, 1H), 5.68 (s, 2H), 3.67-3.46
(m, 2H), 0.79 (dd, J = 8.5, 7.4 Hz, 2H), −0.12 (s, 10H).

### Intermediate 157: tert-butyl 6-(((benzyloxy) carbonyl)(methyl)amino)-2-azaspiro[3.3]heptane-2-carboxylate

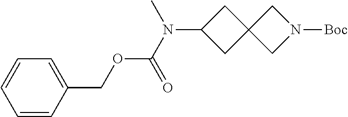 [SMILES: CN(C(=O)OCc1ccccc1)C1CC2(C1)CN(C(=O)OC(C)(C)C)C2]

To a stirred solution of tert-butyl 6-(((benzyloxy) carbonyl)amino)-2-azaspiro[3.3]heptane-2-carboxylate (30.7 g, 89 mmol) in DMF (296 mL) was added NaH (60% in mineral oil) (7.10 g, 178 mmol). After 15 min of stirring the MeI (16.58 ml, 266 mmol) was added and the reaction mixture was stirred at rt for 1 h. Then the mixture was diluted with water and extracted with AcOEt. Organic layer was dried over Na₂SO₄ and concentrated, to give title compound (33.6 g, quantitative).

LC-MS (ESI): Method 10 t_{R}=2.56 min, m/z (M+1)=361.15

¹H NMR (300 MHz, Chloroform-d) & 7.35 (s, 5H), 5.11 (s, 2H), 3.94 (s, 2H), 3.83 (s, 2H), 2.83 (s, 3H), 2.37 (ddt, J=10.2, 7.7, 2.6 Hz, 2H), 2.26 (td, J=9.6, 2.7 Hz, 2H), 1.42 (s, 9H).

### Intermediate 83: (1-methyl-1H-imidazo[1,2-b]pyrazol-7-yl) (6-((methyl-d₃)amino)-2-azaspiro[3.3]heptan-2-yl) methanone

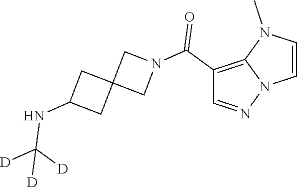 [SMILES: [2H]C([2H])([2H])NC1CC2(C1)CN(C(=O)c1cnn3ccn(C)c13)C2]

### Step 1—benzyl(methyl-d₃)(2-(1-methyl-1/I-imidazo[1,2-b]pyrazole-7-carbonyl)-2-azaspiro[3.3]heptan-6-yl)carbamate (Intermediate 84)

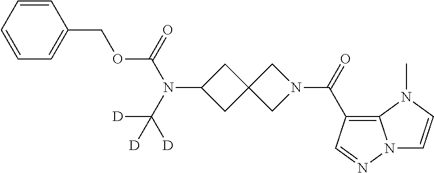 [SMILES: [2H]C([2H])([2H])N(C(=O)OCc1ccccc1)C1CC2(C1)CN(C(=O)c1cnn3ccn(C)c13)C2]

A solution of Intermediate 51 (250 mg, 0.635 mmol) in THF (2.0 mL) was added dropwise under a nitrogen atmosphere to a slurry of sodium hydride (60% suspension in mineral oil, 51 mg, 1.3 mmol) in THF (0.50 mL). The mixture was stirred at rt for 2 h, then iodomethane-d₃ (0.40 mL, 6.4 mmol) was added dropwise. The reaction was then stirred at rt for 18 h. The mixture was carefully quenched with water and was diluted with brine and partitioned with EtOAc. Organic layer was washed with brine, dried over MgSO₄, filtered and solvents removed in vacuo. The crude was purified by FCC (40 g silica gel, 0-10% MeOH in DCM) to yield the title compound (247 mg, 0.60 mmol, 95%).

LC-MS (ESI): Method 1, t_{R}=1.44 min, m/z (M+1)=411.3

The following compound was prepared analogously to what described for Intermediate 84, by starting from suitable Intermediate (see Table 37).

TABLE 37

| | | Product Amount |
|---|---|---|
| | Structure | (Yield) |
| N° | Analytical Data | |
| Intermediate 85 | 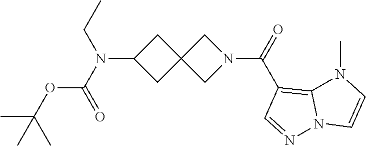 [SMILES: CCN(C(=O)OC(C)(C)C)C1CC2(C1)CN(C(=O)c1cnn3ccn(C)c13)C2] | 165 mg (76%) |
| | LCMS (ESI): Method 1, t_{R} = 1.58 min, m/z (M + 1) = 388.2 | |
| Intermediate 181 | 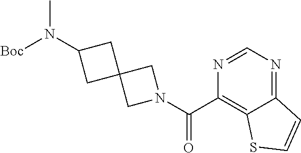 [SMILES: CN(C(=O)OC(C)(C)C)C1CC2(C1)CN(C(=O)c1ncnc3ccsc13)C2] | 1.3 g (quantitative) |

LCMS (ESI): Method 15, t_{R} = 1.73 min, m/z (M + 1) = 389.1

### Step 2—(1-methyl-1H-imidazo[1,2-b]pyrazol-7-yl) (6-((methyl-d₃)amino)-2-azaspiro[3.3]heptan-2-yl) methanone (Intermediate 83)

A suspension of Intermediate 84 (247 mg, 0.602 mmol) and palladium (10% on carbon, 64 mg, 0.0602 mmol) in EtOH (5.0 mL) was subjected to cycles of vacuum/nitrogen, followed by cycles of vacuum/hydrogen before being subjected to 1 atmosphere of hydrogen gas. The reaction was stirred at rt for 18 h. The mixture was filtered on Celite® washing with EtOAc and solvents were removed in vacuo to yield the title compound (161 mg, 0.583 mmol, 97%) which was used without any further purification.

LC-MS (ESI): Method 1 t_{R}=1.04 min, m/z (M+1)=277.2

### Intermediate 86: 1-(2-methoxy-5-(trifluoromethoxy)phenyl)-3-(2azaspiro[3.3]heptan-6-yl)urea

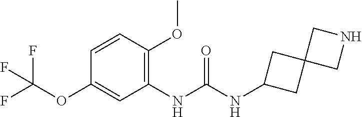 [SMILES: COc1ccc(OC(F)(F)F)cc1NC(=O)NC1CC2(CNC2)C1]

### Step 1—tert-butyl 6-(3-(2-methoxy-5-(trifluoromethoxy)phenyl) ureido)-2-azaspiro[3.3]heptane-2-carboxylate (Intermediate 87)

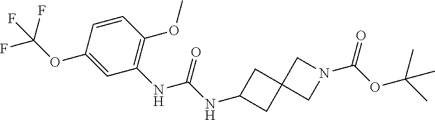 [SMILES: COc1ccc(OC(F)(F)F)cc1NC(=O)NC1CC2(C1)CN(C(=O)OC(C)(C)C)C2]

A solution of 2,2,2-trichloroethyl(2-methoxy-5-(trifluoromethoxy)phenyl)carbamate (649 mg, 1.70 mmol), tert-butyl 6-amino-2-azaspiro[3.3]heptane-2-carboxylate (300 mg, 1.41 mmol) and DIPEA (0.74 mL, 4.24 mmol) in ACN (5.0 mL) was stirred under microwave radiation at 150° C. for 30 minutes, then cooled and concentrated in vacuo to give the title compound (882 mg, 1.98 mmol, quant.).

LC-MS (ESI): Method 1 t_{R}=1.76 min, m/z (M+1-tBu)=390.1

The following Intermediates were prepared analogously to the procedure described for Intermediate 87 (see Table 38).

TABLE 38

| | | Product Amount |
|---|---|---|
| | Structure | (Yield) |
| N° | Analytical Data | |
| Intermediate 88 | 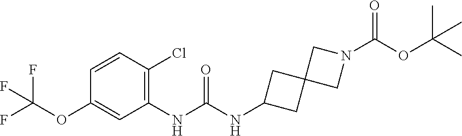 [SMILES: CC(C)(C)OC(=O)N1CC2(CC(NC(=O)Nc3cc(OC(F)(F)F)ccc3Cl)C2)C1] | 400 mg (quantitative) |
| | LCMS (ESI): Method 1, t_{R} = 1.83 min, m/z (M + 1-tBu) = 394.0 | |
| Intermediate 89 | 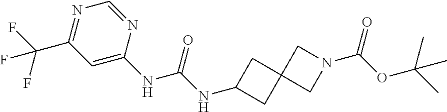 [SMILES: CC(C)(C)OC(=O)N1CC2(CC(NC(=O)Nc3cc(C(F)(F)F)ncn3)C2)C1] | 224 mg (quantitative) |
| | LCMS (ESI): Method 1, t_{R} = 1.61 min, m/z (M-1) = 400.2 | |
| Intermediate 90 | 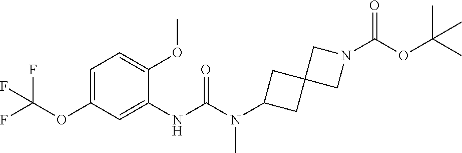 [SMILES: COc1ccc(OC(F)(F)F)cc1NC(=O)N(C)C1CC2(C1)CN(C(=O)OC(C)(C)C)C2] | 160 mg (39%) |
| | LCMS (ESI): Method 1, t_{R} = 1.85 min, m/z (M + 1-tBu) = 403.8 | |
| Intermediate 91 | 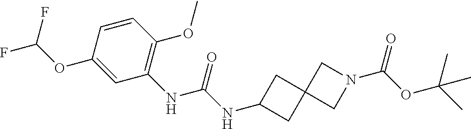 [SMILES: COc1ccc(OC(F)F)cc1NC(=O)NC1CC2(C1)CN(C(=O)OC(C)(C)C)C2] | 340 mg (quantitative) |
| | LCMS (ESI): Method 1, t_{R} = 1.69 min, m/z (M + 1-tBu) = 372.1 | |
| Intermediate 92 | 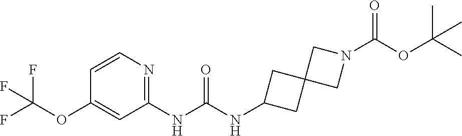 [SMILES: CC(C)(C)OC(=O)N1CC2(CC(NC(=O)Nc3cc(OC(F)(F)F)ccn3)C2)C1] | 750 mg (quantitative) |
| | LCMS (ESI): Method 1, t_{R} = 1.73 min, m/z (M + 1-tBu) = 361.0 | |
| Intermediate 158 | 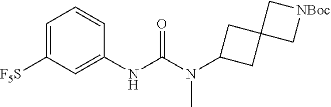 | 556 mg (quantitative) |
| | LCMS (ESI): Method 1, t_{R} = 1.85 min, m/z (M-1) = 470.2 | |
| Intermediate 159 | 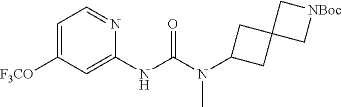 [SMILES: CN(C(=O)Nc1cc(OC(F)(F)F)ccn1)C1CC2(C1)CN(C(=O)OC(C)(C)C)C2] | 488 mg (quantitative) |
| | LCMS (ESI): Method 1, t_{R} = 1.78 min, m/z (M-1) = 431.1 | |
| Intermediate 160 | 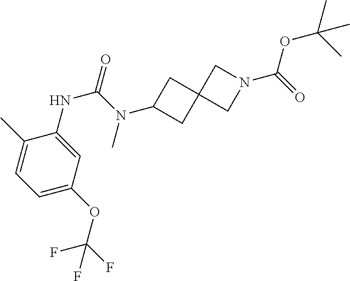 [SMILES: Cc1ccc(OC(F)(F)F)cc1NC(=O)N(C)C1CC2(C1)CN(C(=O)OC(C)(C)C)C2] | 435 mg (70%) |
| | LCMS (ESI): Method 13, t_{R} = 2.33 min, m/z (M + 1-tBu) = 388.2 | |
| Intermediate 161 | 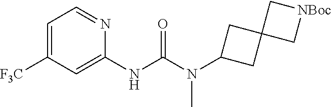 [SMILES: CN(C(=O)Nc1cc(C(F)(F)F)ccn1)C1CC2(C1)CN(C(=O)OC(C)(C)C)C2] | 472 mg (quantitative) |
| | LCMS (ESI): Method 1, t_{R} = 1.78 min, m/z (M + 1) = 415.1 | |
| Intermediate 162 | 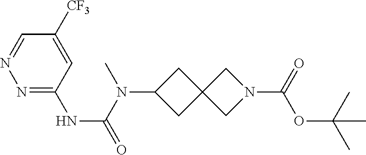 [SMILES: CN(C(=O)Nc1cc(C(F)(F)F)cnn1)C1CC2(C1)CN(C(=O)OC(C)(C)C)C2] | 191 mg (quantitative) |
| | LCMS (ESI): Method 7, t_{R} = 1.07 min, m/z (M + 1) = 414.26 | |
| Intermediate 163 | 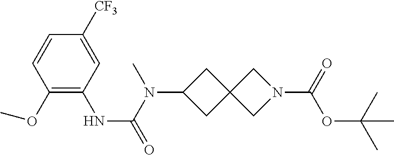 [SMILES: COc1ccc(C(F)(F)F)cc1NC(=O)N(C)C1CC2(C1)CN(C(=O)OC(C)(C)C)C2] | 360 mg (quantitative) |

LCMS (ESI): Method 7, t_{R} = 1.28 min, m/z (M-1) = 442.22

### Step 2—1-(2-methoxy-5-(trifluoromethoxy)phenyl)-3-(2-azaspiro[3.3]heptan-6-yl)urea (Intermediate 86)

Intermediate 86 was prepared by following the procedure described for Intermediate 7.

SM: Previous Intermediate: 882 mg (1 eq); TFA: 3.0 mL (20 eq)

Amount/yield: 450 mg, 1.79 mmol, 66%

LC-MS (ESI): Method 1 t_{R}=1.32 min, m/z (M+1-)=346.0

The following compounds were prepared analogously to what described for Intermediate 86, starting from previously described intermediates (see Table 39).

TABLE 39

| | | Product Amount |
|---|---|---|
| | Structure | (Yield) |
| N° | Analytical Data | |
| Intermediate 93 | 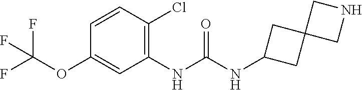 [SMILES: O=C(Nc1cc(OC(F)(F)F)ccc1Cl)NC1CC2(CNC2)C1] | 100 mg (32%) |
| | LCMS (ESI): Method 1, t_{R} = 1.39 min, m/z (M + 1) = 350.0 | |
| Intermediate 94 | 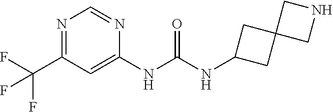 [SMILES: O=C(Nc1cc(C(F)(F)F)ncn1)NC1CC2(CNC2)C1] | 107 mg (75%) |
| | LCMS (ESI): Method 4, t_{R} = 1.14 min, m/z (M + 1) = 302.1 | |
| Intermediate 95 | 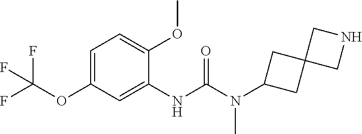 [SMILES: COc1ccc(OC(F)(F)F)cc1NC(=O)N(C)C1CC2(CNC2)C1] | 117 mg (94%) |
| | LCMS (ESI): Method 1, t_{R} = 1.38 min, m/z (M + 1) = 359.8 | |
| Intermediate 96 | 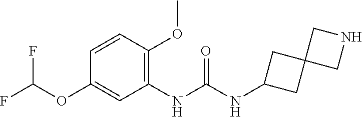 [SMILES: COc1ccc(OC(F)F)cc1NC(=O)NC1CC2(CNC2)C1] | 100 mg (38%) |
| | LCMS (ESI): Method 1, t_{R} = 1.26 min, m/z (M + 1) = 328.0 | |
| Intermediate 97 | 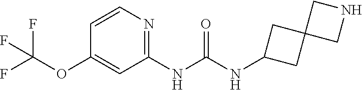 [SMILES: O=C(Nc1cc(OC(F)(F)F)ccn1)NC1CC2(CNC2)C1] | 350 mg (61%) |
| | LCMS (ESI): Method 1, t_{R} = 1.21 min, m/z (M + 1) = 316.8 | |
| Intermediate 164 | 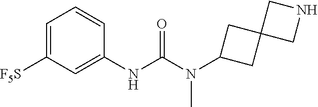 | 217 mg (quantitative) |
| | LCMS (ESI): Method 1, t_{R} = 1.28 min, m/z (M + 1) = 372.0 | |
| Intermediate 165 | 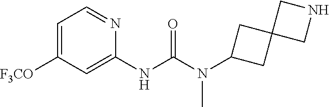 [SMILES: CN(C(=O)Nc1cc(OC(F)(F)F)ccn1)C1CC2(CNC2)C1] | 164 mg (quantitative) |
| | LCMS (ESI): Method 1, t_{R} = 1.15 min, m/z (M + 1) = 331.0 | |
| Intermediate 166 | 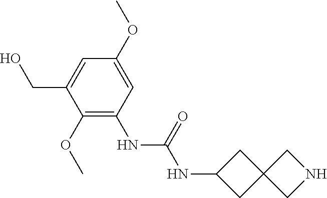 [SMILES: COc1cc(CO)c(OC)c(NC(=O)NC2CC3(CNC3)C2)c1] | 207 mg (quantitative) |
| | LCMS (ESI): Method 14, t_{R} = 1.62 min, m/z (M + 1) = 321.6 | |
| | 1H NMR (300 MHz, DMSO-d6) δ 8.14 (d, J = 3.3 Hz, 1H), 8.09 (d, J = 3.1 Hz, | |
| | 1H), 7.25 (t, J = 8.2 Hz, 1H), 6.89 (d, J = 3.0 Hz, 1H), 5.32 (t, J = 5.7 Hz, 1H), | |
| | 4.55 (d, J = 5.5 Hz, 2H), 4.06 (dddd, J = 21.1, 14.0, 9.6, 5.8 Hz, 6H), 3.66 (d, J = | |
| | 1.2 Hz, 3H), 2.13-1.95 (m, 2H), 1.73 (d, J = 3.5 Hz, 1H) | |
| Intermediate 167 | 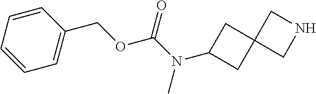 [SMILES: CN(C(=O)OCc1ccccc1)C1CC2(CNC2)C1] | 1.3 g (57%) |
| | LCMS (ESI): Method 10, t_{R} = 1.76 min, m/z (M + 1) = 261.15 | |
| | 1H NMR (300 MHz, DMSO-d6) δ 7.41-7.28 (m, 5H), 5.05 (s, 2H), 4.01 (s, 2H), | |
| | 3.87 (s, 2H), 3.17 (s, 4H), 2.78 (s, 3H), 2.41-2.32 (m, 4H). | |
| Intermediate 168 | 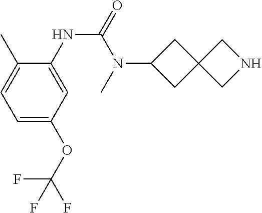 [SMILES: Cc1ccc(OC(F)(F)F)cc1NC(=O)N(C)C1CC2(CNC2)C1] | 358 mg (80%) |
| | LCMS (ESI): Method 7, t_{R} = 0.57 min, m/z (M + 1) = 344.14 | |
| Intermediate 169 | 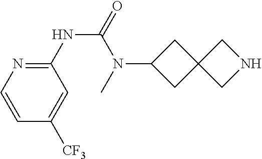 [SMILES: CN(C(=O)Nc1cc(C(F)(F)F)ccn1)C1CC2(CNC2)C1] | 284 mg (100%) |
| | LCMS (ESI): Method 7, t_{R} = 0.44 min, m/z (M + 1) = 315.04 | |
| Intermediate 170 | 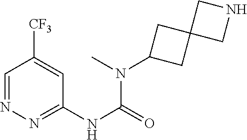 [SMILES: CN(C(=O)Nc1cc(C(F)(F)F)cnn1)C1CC2(CNC2)C1] | 197 mg (100%) |
| | LCMS (ESI): Method 7, t_{R} = 0.33 min, m/z (M + 1) = 316.09 | |
| Intermediate 171 | 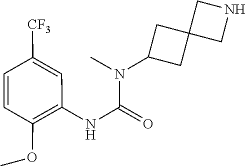 [SMILES: COc1ccc(C(F)(F)F)cc1NC(=O)N(C)C1CC2(CNC2)C1] | 197 mg (100%) |

LCMS (ESI): Method 7, t_{R} = 0.33 min, m/z (M + 1) = 344.04

### Intermediate 172: benzyl methyl(2-(1-methyl-1H-imidazo[1,2-b]pyrazole-7-carbonyl)-2-azaspiro[3.3]heptan-6-yl)carbamate

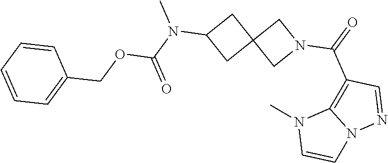 [SMILES: CN(C(=O)OCc1ccccc1)C1CC2(C1)CN(C(=O)c1cnn3ccn(C)c13)C2]

1-methyl-1H-imidazo[1,2-b]pyrazole-7-carboxylic acid (0.866 g, 5.2 mmol) was dissolved in DMF (25 mL) and then added of CDI. Solution was stirred a rt until formation of activated ester then Intermediate 167 (1.3 g, 5 mmol) was added to the solution and stirred at rt on. Solution was then diluted with EtOAc and washed with NaHCO₃ sat solution. Organic phase was then evaporated under vacuum and purified by FCC (Sfar NH, from 100% of n-heptane to 40% of EtOac/EtOH 3:1). Appropriate fractions were combined and evaporated under vacuum to give title compound (550 mg, 1.3 mmol, 27% yield). 10

LCMS (ESI): Method 2, t_{R}-0.92 min, m/z (M+1)=408.19

### Intermediate 173: tert-butyl 6-(((benzyloxy) carbonyl)amino)-2-azaspiro[3.3]heptane-2-carboxylate

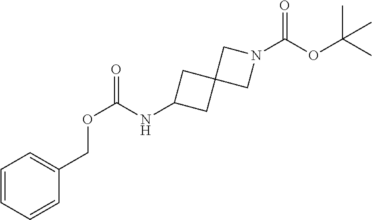 [SMILES: CC(C)(C)OC(=O)N1CC2(CC(NC(=O)OCc3ccccc3)C2)C1]

Tert-butyl 6-amino-2-azaspiro[3.3]heptane-2-carboxylate (30 g, 141 mmol)) and triethylamine (21.7 ml, 155 mmol) were dissolved in DCM (70 mL), the solution was cooled at −5° C. and stirred for 10 min, then N-(benzyloxycarbonyloxy) succinimide (37.0 g, 148 mmol) was added carefully. The reaction mixture was stirred at rt until completion, then it was washed with sol. NH₄Cl and HCl 0.5N, the organic layer was dried over MgSO₄, filtered and essicated to afford title compound (48 g, 139 mmol, 98% yield).

LCMS (ESI): Method 2, t_{R}-0.92 min, m/z (M+1)=408.19

### Example 52: 3-(2-methoxy-5-(trifluoromethoxy)phenyl)-1-methyl-1-(2-(1-methyl-1H-imidazo[1,2-b]pyrazole-7-carbonyl)-2-azaspiro[3.3]heptan-6-yl)urea

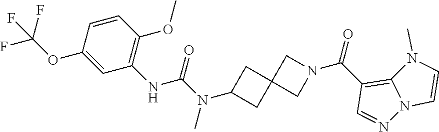 [SMILES: COc1ccc(OC(F)(F)F)cc1NC(=O)N(C)C1CC2(C1)CN(C(=O)c1cnn3ccn(C)c13)C2]

### Example 52

To a stirred solution of Intermediate 57 (70 mg, 0.26 mmol) and 2,2,2-trichloroethyl(2-methoxy-5-(trifluoromethoxy)phenyl)carbamate (147 mg, 0.38 mmol) in ACN (3 mL) DIPEA (0.13 mL, 0.77 mmol) was added and the reaction mixture was heated to 150° C. in the microwave for 30 min. The reaction mixture was concentrated in vacuo to dryness. The crude residue was purified by preparative HPLC (Sunfire C18 19×150 mm, 10 μm 20-80% ACN/H₂O (0.1% FA), 20 mL/minute) to give the title compound (73 mg, 0.144 mmol, 56% yield).

LCMS (ESI): Method 2, t_{R}=4.42 min, m/z (M+1)=507.2

¹H NMR (400 MHz, DMSO-d6) δ 8.00 (d, J=2.0 Hz, 1H), 7.83 (s, 1H), 7.72 (d, J=2.0 Hz, 1H), 7.51 (s, 1H), 7.32 (d, J=1.3 Hz, 1H), 7.13 (d, J=8.8 Hz, 1H), 7.01 (dd, J=2.3, 8.8 Hz, 1H), 4.58-4.48 (m, 1H), 4.39-4.08 (m, 4H), 4.02 (s, 3H), 3.94 (s, 3H), 2.94 (s, 3H), 2.54-2.37 (m, 4H).

The following Examples and intermediates were prepared analogously to what described for Example 52 using ACN or DMF as solvent, at a temperature varying from 80 to 150° C., starting from suitable Troc carbamates and Intermediate (see Table 40)

TABLE 40

| | | Product Amount |
|---|---|---|
| | Structure | (Yield) |
| N^{o} | Analytical Data | |
| Example 43 | 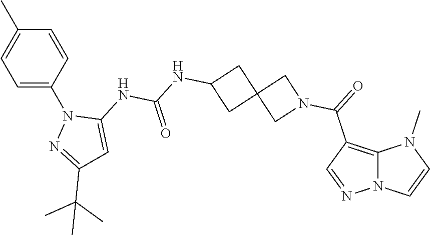 [SMILES: Cc1ccc(-n2nc(C(C)(C)C)cc2NC(=O)NC2CC3(C2)CN(C(=O)c2cnn4ccn(C)c24)C3)cc1] | 60 mg (46%) |
| | LCMS (ESI): Method 3, t_{R} = 4.26 min, m/z (M + 1) = 515.5 ¹H NMR (400 MHz, DMSO-d6) δ | |
| | 7.98 (s, 1H), 7.75 (s, 1H), 7.65 (d, J = 2.3 Hz, 1H), 7.35-7.28 (m, 4H), 7.25-7.24 (m, 1H), 6.79 (d, | |
| | J = 7.3 Hz, 1H), 6.22 (s, 1H), 4.37-3.95 (m, 8H), 2.36 (s, 3H), 2.08-1.99 (m, 2H), 1.25 (s, 9H). | |
| Example 59 | 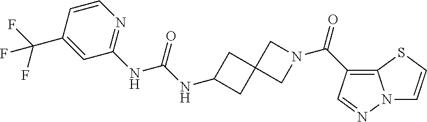 [SMILES: O=C(Nc1cc(C(F)(F)F)ccn1)NC1CC2(C1)CN(C(=O)c1cnn3ccsc13)C2] | 33 mg (32%) |
| | LCMS (ESI): Method 2, t_{R} = 3.89 min, m/z (M + 1) = 451.1 ¹H NMR (400 MHz, DMSO-d6) δ | |
| | 9.45 (s, 1H), 8.47 (d, H = 5.3 Hz, 1H), 8.32 (d, J = 4.0 Hz, 1H), 8.08 (s, 1H), 7.95 (s, 1H), 7.66 | |
| | (d, J = 7.2 Hz, 1H), 7.53 (d, J = 3.0 Hz, 1H), 7.27 (d, J = 4.8 Hz, 1H), 4.57-4.39 (m, 2H), 4.14- | |
| | 4.01 (m, 3H), 2.64-2.54 (m, 2H), 2.21-2.12 (m, 2H). | |
| Example 61 | 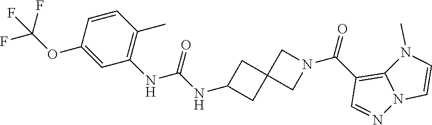 [SMILES: Cc1ccc(OC(F)(F)F)cc1NC(=O)NC1CC2(C1)CN(C(=O)c1cnn3ccn(C)c13)C2] | 81 mg (55%) |
| | LCMS (ESI): Method 2, t_{R} = 4.21 min, m/z (M + 1) = 477.3 ¹H NMR (400 MHz, DMSO-d6) δ | |
| | 8.02 (d, J = 1.3 Hz, 1H), 7.77 (d, J = 1.3 Hz, 1H), 7.73 (s, 1H), 7.66 (d, J = 2.0 Hz, 1H), 7.25 | |
| | (dd, J = 1.3, 2.0 Hz, 1H), 7.21 (d, J = 8.8 Hz, 1H), 7.04 (d, J = 7.3 Hz, 1H), 6.81 (dd, J = 1.6, 8.5 | |
| | Hz, 1H), 4.40-4.00 (m, 5H), 3.96 (s, 3H), 2.59-2.52 (m, 2H), 2.18 (s, 3H), 2.11-2.04 (m, 2H). | |
| Example 63 | 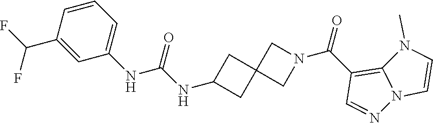 [SMILES: Cn1ccn2ncc(C(=O)N3CC4(CC(NC(=O)Nc5cccc(C(F)F)c5)C4)C3)c12] | 45 mg (34%) |
| | LCMS (ESI): Method 3, t_{R} = 3.70 min, m/z (M + 1) = 429.4 ¹H NMR (400 MHz, DMSO-d6) δ | |
| | 8.63 (s, 1H), 7.78 (s, 1H), 7.71 (s, 1H), 7.67 (d, J = 2.1 Hz, 1H), 7.44 (d, J = 7.9 Hz, 1H), 7.35 | |
| | (t, J = 7.8 Hz, 1H), 7.26-7.26 (m, 1H), 7.08 (td, J = 1.4, 7.7 Hz, 1H), 6.96 (t, J = 56.1 Hz, 1H), | |
| | 6.52 (d, J = 7.7 Hz, 1H), 4.51-4.02 (m, 5H), 3.97 (s, 3H), 2.57-2.52 (m, 2H), 2.16-2.09 (m, 2H). | |
| Example 65 | 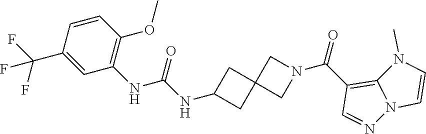 [SMILES: COc1ccc(C(F)(F)F)cc1NC(=O)NC1CC2(C1)CN(C(=O)c1cnn3ccn(C)c13)C2] | 76 mg (51%) |
| | LCMS (ESI): Method 3, t_{R} = 4.09 min, m/z (M + 1) = 477.3 ¹H NMR (400 MHz, DMSO-d6) δ | |
| | 8.49 (d, J = 2.3 Hz, 1H), 8.09 (s, 1H), 7.77 (d, J = 1.3 Hz, 1H), 7.66 (d, J = 2.3 Hz, 1H), 7.29- | |
| | 7.21 (m, 3H), 7.14 (d, J = 8.3 Hz, 1H), 4.48-3.98 (m, 5H), 3.96 (s, 3H), 3.92 (s, 3H), 2.59-2.52 | |
| | (m, 2H), 2.08-2.01 (m, 2H). | |
| Example 66 | 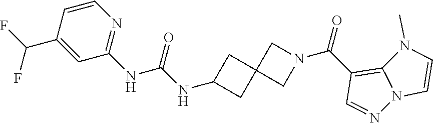 [SMILES: Cn1ccn2ncc(C(=O)N3CC4(CC(NC(=O)Nc5cc(C(F)F)ccn5)C4)C3)c12] | 59 mg (44%) |
| | LCMS (ESI): Method 2, t_{R} = 3.34 min, m/z (M + 1) = 430.3 ¹H NMR (400 MHz, DMSO-d6) δ | |
| | 9.31 (s, 1H), 8.34 (d, J = 5.3 Hz, 1H), 7.88 (d, J = 7.1 Hz, 1H), 7.77 (s, 1H), 7.69 (s, 1H), 7.66 | |
| | (d, J = 2.3 Hz, 1H), 7.26 (d, J = 1.0 Hz, 1H), 7.08 (d, J = 4.8 Hz, 1H), 7.05 (t, J = 55.4 Hz, 1H), | |
| | 4.43-4.06 (m, 5H), 3.96 (s, 3H), 2.60-2.53 (m, 2H), 2.17-2.07 (m, 2H). | |
| Example 67 | 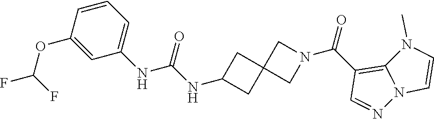 [SMILES: Cn1ccn2ncc(C(=O)N3CC4(CC(NC(=O)Nc5cccc(OC(F)F)c5)C4)C3)c12] | 42 mg (31%) |
| | LCMS (ESI): Method 2, t_{R} = 3.68 min, m/z (M + 1) = 445.3 ¹H NMR (400 MHz, DMSO-d6) δ | |
| | 8.59 (s, 1H), 7.77 (d, J = 1.0 Hz, 1H), 7.66 (d, J = 2.0 Hz, 1H), 7.39 (dd, J = 2.1, 2.1 Hz, 1H), | |
| | 7.27-7.22 (m, 1H), 7.15 (t, J = 74.4 Hz, 1H), 7.11 (dd, J = 1.1, 8.2 Hz, 1H), 6.68 (dd, J = 2.3, | |
| | 8.1 Hz, 1H), 6.53-6.48 (m, 1H), 4.35-4.00 (m, 5H), 3.96 (s, 3H), 2.54 (dd, J = 2.9, 7.2 Hz, | |
| | 2H), 2.14-2.07 (m, 2H). | |
| Example 71 | 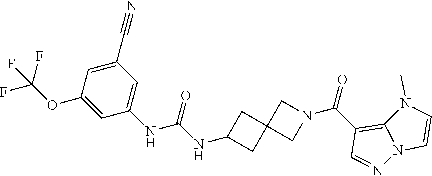 [SMILES: Cn1ccn2ncc(C(=O)N3CC4(CC(NC(=O)Nc5cc(C#N)cc(OC(F)(F)F)c5)C4)C3)c12] | 48 mg (32%) |
| | LCMS (ESI): Method 2, t_{R} = 4.13 min, m/z (M + 1) = 488.3 ¹H NMR (400 MHz, DMSO-d6) δ | |
| | 9.07 (s, 1H), 7.87 (s, 1H), 7.77 (s, 2H), 7.66 (d, J = 2.3 Hz, 1H), 7.47 (s, 1H), 7.26-7.26 (m, 1H), | |
| | 6.86 (d, J = 7.7 Hz, 1H), 4.51-4.02 (m, 5H), 3.97 (s, 3H), 2.57-2.53 (m, 2H), 2.19-2.11 (m, 2H). | |
| Example 72 | 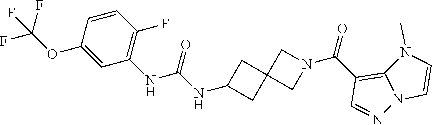 [SMILES: Cn1ccn2ncc(C(=O)N3CC4(CC(NC(=O)Nc5cc(OC(F)(F)F)ccc5F)C4)C3)c12] | 38 mg (26%) |
| | LCMS (ESI): Method 2, t_{R} = 4.22 min, m/z (M + 1) = 481.1 ¹H NMR (400 MHz, DMSO-d6) δ | |
| | 8.51 (d, J = 2.6 Hz, 1H), 8.22 (dd, J = 2.7, 6.5 Hz, 1H), 7.77 (s, 1H), 7.67 (d, J = 2.3 Hz, 1H), | |
| | 7.33 (dd, J = 9.0, 11.0 Hz, 1H), 7.26-7.26 (m, 1H), 7.01 (d, J = 7.4 Hz, 1H), 6.95-6.89 (m, 1H), | |
| | 4.48-4.01 (m, 5H), 3.97 (s, 3H), 2.59-2.53 (m, 2H), 2.12-2.04 (m, 2H). | |
| Example 74 | 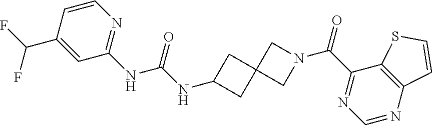 [SMILES: O=C(Nc1cc(C(F)F)ccn1)NC1CC2(C1)CN(C(=O)c1ncnc3ccsc13)C2] | 46 mg (47%) |
| | LCMS (ESI): Method 2, t_{R} = 4.02 min, m/z (M + 1) = 445.2 ¹H NMR (400 MHz, DMSO-d6) δ | |
| | 9.33-9.27 (m, 2H), 8.59 (d, J = 5.5 Hz, 1H), 8.34 (d, J = 5.3 Hz, 1H), 7.87 (dd, J = 6.9, 6.9 Hz, | |
| | 1H), 7.72-7.67 (m, 2H), 7.08 (d, J = 5.1 Hz, 1H), 7.05 (t, J = 55.2 Hz, 1H), 4.87 (s, 1H), 4.77 | |
| | (s, 1H), 4.26 (s, 1H), 4.17-4.07 (m, 2H), 2.68-2.54 (m, 2H), 2.21-2.14 (m, 2H). | |
| Example 75 | 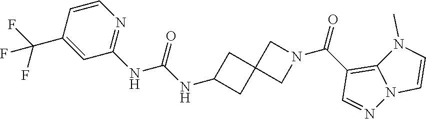 [SMILES: Cn1ccn2ncc(C(=O)N3CC4(CC(NC(=O)Nc5cc(C(F)(F)F)ccn5)C4)C3)c12] | 83 mg (50%) |
| | LCMS (ESI): Method 2, t_{R} = 3.78 min, m/z (M + 1) = 448.34 ¹H NMR (400 MHz, DMSO-d6) δ | |
| | 9.43 (s, 1H), 8.45 (d, J = 5.3 Hz, 1H), 7.93 (s, 1H), 7.77 (d, J = 1.1 Hz, 1H), 7.67-7.63 (m, 2H), | |
| | 7.28-7.25 (m, 2H), 4.19-3.96 (m, 8H), 2.60-2.52 (m, 2H), 2.17-2.10 (m, 2H). | |
| Example 76 | 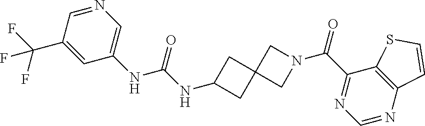 [SMILES: O=C(Nc1cncc(C(F)(F)F)c1)NC1CC2(C1)CN(C(=O)c1ncnc3ccsc13)C2] | 42 mg (50%) |
| | LCMS (ESI): Method 2, t_{R} = 3.99 min, m/z (M + 1) = 463.4 ¹H NMR (400 MHz, DMSO-d6) δ | |
| | 9.29 (d, J = 7.5 Hz, 1H), 9.04 (d, J = 3.9 Hz, 1H), 8.72 (d, J = 2.1 Hz, 1H), 8.60 (d, J = 5.5 Hz, | |
| | 1H), 8.48 (s, 1H), 8.39-8.36 (m, 1H), 7.69 (dd, J = 2.0, 5.5 Hz, 1H), 6.87 (d, J = 7.4 Hz, 1H), | |
| | 4.87 (s, 1H), 4.77 (s, 1H), 4.26 (s, 1H), 4.16 (s, 1H), 4.12-4.03 (m, 1H), 2.63-2.54 (m, 2H), | |
| | 2.24-2.17 (m, 2H). | |
| Example 78 | 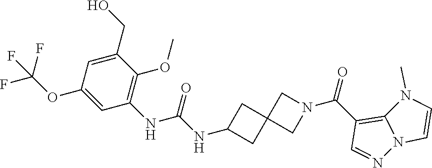 [SMILES: COc1c(CO)cc(OC(F)(F)F)cc1NC(=O)NC1CC2(C1)CN(C(=O)c1cnn3ccn(C)c13)C2] | 29 mg (23%) |
| | LCMS (ESI): Method 2, t_{R} = 3.61 min, m/z (M + 1) = 523.36 ¹H NMR (400 MHz, DMSO-d6) δ | |
| | 8.15 (s, 1H), 8.10 (d, J = 2.8 Hz, 1H), 7.77 (d, J = 1.0 Hz, 1H), 7.66 (d, J = 2.3 Hz, 1H), 7.30- | |
| | 7.25 (m, 2H), 6.90-6.88 (m, 1H), 5.32-5.26 (m, 1H), 4.56-3.96 (m, 10H), 3.67 (s, 3H), 2.59- | |
| | 2.53 (m, 2H), 2.10-2.03 (m, 2H). | |
| Example 80 | 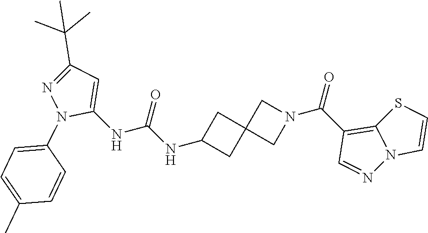 [SMILES: Cc1ccc(-n2nc(C(C)(C)C)cc2NC(=O)NC2CC3(C2)CN(C(=O)c2cnn4ccsc24)C3)cc1] | 35 mg (36%) |
| | LCMS (ESI): Method 2, t_{R} = 4.36 min, m/z (M + 1) = 518.7 ¹H NMR (400 MHz, DMSO-d6) δ | |
| | 8.31 (d, J = 4.1 Hz, 1H), 8.06 (s, 1H), 7.98 (s, 1H), 7.52 (dd, J = 1.3, 4.1 Hz, 1H), 7.35-7.34 (m, | |
| | 1H), 7.33-7.28 (m, 3H), 6.80 (d, J = 7.4 Hz, 1H), 6.23 (s, 1H), 4.57-4.25 (m, 2H), 4.18-3.90 (m, | |
| | 3H), 2.36 (s, 3H), 2.08-2.01 (m, 2H), 1.25 (s, 9H). 2H obscured by DMSO peak. | |
| Example 82 | 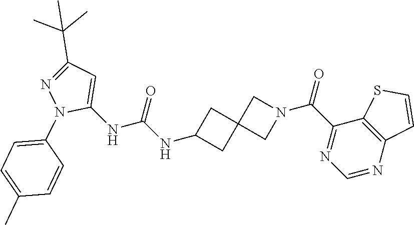 [SMILES: Cc1ccc(-n2nc(C(C)(C)C)cc2NC(=O)NC2CC3(C2)CN(C(=O)c2ncnc4ccsc24)C3)cc1] | 59 mg (34%) |
| | LCMS (ESI): Method 2, t_{R} = 4.85 min, m/z (M + 1) = 530.6 ¹H NMR (400 MHz, DMSO-d6) δ | |
| | 9.28 (d, J = 8.3 Hz, 1H), 8.60 (d, J = 5.5 Hz, 1H), 7.99 (s, 1H), 7.69 (dd, J = 2.0, 5.5 Hz, 1H), | |
| | 7.36-7.35 (m, 1H), 7.32 (d, J = 5.3 Hz, 3H), 6.81 (d, J = 7.3 Hz, 1H), 6.24 (s, 1H), 4.84 (s, 1H), | |
| | 4.73 (s, 1H), 4.23 (s, 1H), 4.12 (s, 1H), 4.01-3.93 (m, 1H), 2.61-2.54 (m, 2H), 2.37 (d, J = 2.0 | |
| | Hz, 3H), 2.10-2.05 (m, 2H), 1.25 (s, 9H). | |
| Example 83 | 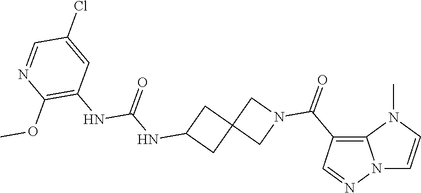 [SMILES: COc1ncc(Cl)cc1NC(=O)NC1CC2(C1)CN(C(=O)c1cnn3ccn(C)c13)C2] | 80 mg (49%) |
| | LCMS (ESI): Method 2, t_{R} = 3.82 min, m/z (M + 1) = 446.1 ¹H NMR (400 MHz, DMSO-d6) δ | |
| | 8.41 (d, J = 2.5 Hz, 1H), 8.16 (s, 1H), 7.76 (d, J = 1.1 Hz, 1H), 7.72 (d, J = 2.5 Hz, 1H), 7.66 (d, | |
| | J = 2.1 Hz, 1H), 7.28 (d, J = 7.4 Hz, 1H), 7.26-7.25 (m, 1H), 4.18-3.94 (m, 11H), 2.59-2.52 (m, | |
| | 2H), 2.08-2.02 (m, 2H). | |
| Example 89 | 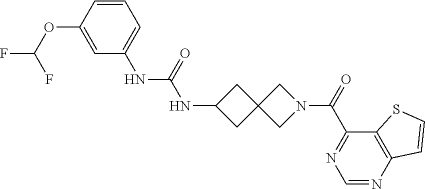 [SMILES: O=C(Nc1cccc(OC(F)F)c1)NC1CC2(C1)CN(C(=O)c1ncnc3ccsc13)C2] | 47 mg (47%) |
| | LCMS (ESI): Method 2, t_{R} = 4.26 min, m/z (M + 1) = 460.2 ¹H NMR (400 MHz, DMSO-d6) δ | |
| | 9.29 (d, J = 7.8 Hz, 1H), 8.60 (d, J = 5.8 Hz, 2H), 7.69 (dd, J = 1.8, 5.4 Hz, 1H), 7.41 (d, J = 1.5 | |
| | Hz, 1H), 7.25 (dd, J = 8.1, 8.1 Hz, 1H), 7.17 (t, J = 74.2 Hz, 1H), 7.11 (d, J = 8.4 Hz, 1H), 6.71- | |
| | 6.68 (m, 1H), 6.51 (d, J = 7.5 Hz, 1H), 4.87 (s, 1H), 4.76 (s, 1H), 4.26 (s, 1H), 4.15 (s, 1H), | |
| | 4.10-4.01 (m, 1H), 2.60-2.55 (m, 2H), 2.19-2.07 (m, 2H). | |
| Example 90 | 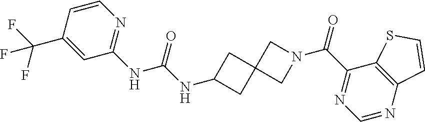 [SMILES: O=C(Nc1cc(C(F)(F)F)ccn1)NC1CC2(C1)CN(C(=O)c1ncnc3ccsc13)C2] | 40 mg (40%) |
| | LCMS (ESI): Method 2, t_{R} = 4.45 min, m/z (M + 1) = 463.5 ¹H NMR (400 MHz, DMSO-d6) δ | |
| | 9.46 (d, J = 4.6 Hz, 1H), 9.29 (d, J = 8.1 Hz, 1H), 8.60 (d, J = 5.5 Hz, 1H), 8.47 (d, J = 5.3 Hz, | |
| | 1H), 7.96-7.95 (m, 1H), 7.71-7.64 (m, 2H), 7.28 (d, J = 5.1 Hz, 1H), 4.88 (s, 1H), 4.78 (s, 1H), | |
| | 4.27 (s, 1H), 4.18-4.07 (m, 2H), 2.66-2.57 (m, 2H), 2.21-2.15 (m, 2H). | |
| Example 94 | 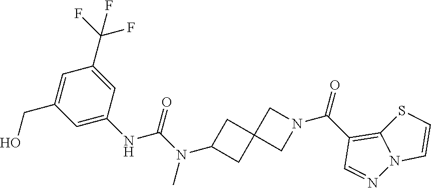 [SMILES: CN(C(=O)Nc1cc(CO)cc(C(F)(F)F)c1)C1CC2(C1)CN(C(=O)c1cnn3ccsc13)C2] | 27 mg (18%) |
| | LCMS (ESI): Method 2, t_{R} = 3.68 min, m/z (M + 1) = 494.2 ¹H NMR (400 MHz, DMSO-d6) δ | |
| | 8.60 (s, 1H), 8.32 (d, J = 4.1 Hz, 1H), 8.08 (s, 1H), 7.81 (s, 1H), 7.71 (s, 1H), 7.53 (d, J = 4.0 | |
| | Hz, 1H), 7.21 (s, 1H), 5.37 (t, J = 5.7 Hz, 1H), 4.57-4.51 (m, 4H), 4.41 (s, 1H), 4.12 (s, 1H), | |
| | 3.99 (m, 1H), 2.89 (s, 3H), 2.47-2.35 (m, 4H). | |
| Example 95 | 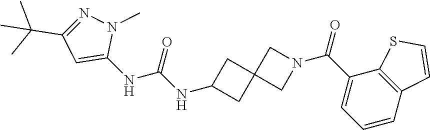 [SMILES: Cn1nc(C(C)(C)C)cc1NC(=O)NC1CC2(C1)CN(C(=O)c1cccc3ccsc13)C2] | 54 mg (55%) |
| | LCMS (ESI): Method 2, t_{R} = 3.71 min, m/z (M + 1) = 454.5 ¹H NMR (400 MHz, DMSO-d6) δ | |
| | 9.28 (d, J = 5.8 Hz, 1H), 8.59 (d, J = 5.5 Hz, 1H), 8.15 (s, 1H), 7.68 (dd, J = 1.6, 5.6 Hz, 1H), | |
| | 6.62 (d, J = 7.3 Hz, 1H), 5.92 (s, 1H), 4.85 (s, 1H), 4.75 (s, 1H), 4.24 (s, 1H), 4.14 (s, 1H), 4.06- | |
| | 3.97 (m, 1H), 3.52 (s, 3H), 2.57-2.54 (m, 2H), 2.33 (s, 1H), 2.18-2.08 (m, 3H), 1.18 (s, 9H). | |
| Example 97 | 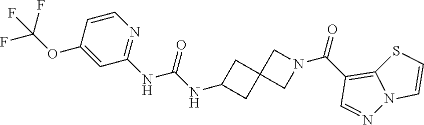 [SMILES: O=C(Nc1cc(OC(F)(F)F)ccn1)NC1CC2(C1)CN(C(=O)c1cnn3ccsc13)C2] | 68 mg (64%) |
| | LCMS (ESI): Method 2, t_{R} = 3.98 min, m/z (M + 1) = 467.3 ¹H NMR (400 MHz, DMSO-d6) δ | |
| | 9.36 (s, 1H), 8.33-8.29 (m, 2H), 8.08 (s, 1H), 7.71 (d, J = 7.3 Hz, 1H), 7.59 (s, 1H), 7.53 (d, J = | |
| | 3.5 Hz, 1H), 6.95 (d, J = 5.1 Hz, 1H), 4.58-4.36 (m, 1H), 4.12-3.97 (m, 3H), 2.62-2.54 (m, 2H), | |
| | 2.19-2.11 (m, 2H). | |
| Example 98 | 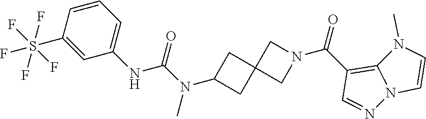 [SMILES: CN(C(=O)Nc1cccc(S(F)(F)(F)(F)F)c1)C1CC2(C1)CN(C(=O)c1cnn3ccn(C)c13)C2] | 46 mg (30%) |
| | LCMS (ESI): Method 2, t_{R} = 4.34 min, m/z (M + 1) = 519.4 ¹H NMR (400 MHz, DMSO-d6) δ | |
| | 8.68 (s, 1H), 8.13 (t, J = 2.1 Hz, 1H), 7.79-7.74 (m, 2H), 7.67 (d, J = 2.3 Hz, 1H), 7.51-7.42 (m, | |
| | 2H), 7.28-7.26 (m, 1H), 4.62-4.52 (m, 1H), 4.20-4.20 (m, 4H), 3.97 (s, 3H), 2.90 (s, 3H), 2.44- | |
| | 2.36 (m, 4H). | |
| Example 100 | 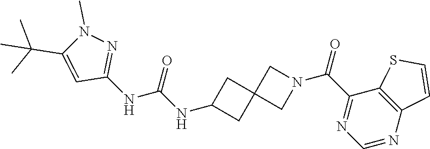 [SMILES: Cn1nc(NC(=O)NC2CC3(C2)CN(C(=O)c2ncnc4ccsc24)C3)cc1C(C)(C)C] | 56 mg (56%) |
| | LCMS (ESI): Method 2, t_{R} = 4.18 min, m/z (M + 1) = 453.6 ¹H NMR (400 MHz, DMSO-d6) δ | |
| | 9.29 (d, J = 8.1 Hz, 1H), 8.60 (d, J = 5.6 Hz, 1H), 8.54 (s, 1H), 7.69 (dd, J = 1.8, 5.6 Hz, 1H), | |
| | 7.04-7.04 (m, 1H), 5.86 (s, 1H), 4.86 (s, 1H), 4.77 (s, 1H), 4.25 (s, 1H), 4.15 (s, 1H), 4.08-3.99 | |
| | (m, 1H), 3.77 (s, 3H), 2.62-2.55 (m, 2H), 2.16-2.08 (m, 2H), 1.30 (s, 9H). | |
| Example 101 | 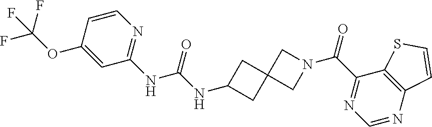 [SMILES: O=C(Nc1cc(OC(F)(F)F)ccn1)NC1CC2(C1)CN(C(=O)c1ncnc3ccsc13)C2] | 18 mg (17%) |
| | LCMS (ESI): Method 2, t_{R} = 4.51 min, m/z (M + 1) = 479.5 ¹H NMR (400 MHz, DMSO-d6) δ | |
| | 9.37-9.34 (m, 1H), 9.29 (d, J = 8.2 Hz, 1H), 8.60 (d, J = 5.6 Hz, 1H), 8.31 (d, J = 5.8 Hz, 1H), | |
| | 7.71-7.66 (m, 2H), 7.59 (d, J = 4.6 Hz, 1H), 6.95 (d, J = 5.6 Hz, 1H), 4.88 (s, 1H), 4.77 (s, 1H), | |
| | 4.26 (s, 1H), 4.17-4.07 (m, 2H), 2.68-2.57 (m, 2H), 2.17 (dd, J = 9.6, 9.6 Hz, 2H). | |
| Example 104 | 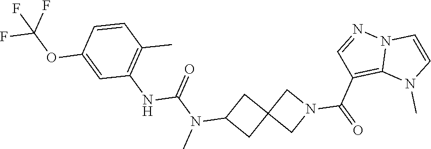 [SMILES: Cc1ccc(OC(F)(F)F)cc1NC(=O)N(C)C1CC2(C1)CN(C(=O)c1cnn3ccn(C)c13)C2] | 31 mg (45%) |
| | LCMS (ESI): Method 2, t_{R} = 4.26 min, m/z (M + 1) = 491.5 ¹H NMR (400 MHz, DMSO-d6) δ | |
| | 7.82 (s, 1H), 7.77 (s, 1H), 7.67 (d, J = 2.2 Hz, 1H), 7.35 (s, 1H), 7.31-7.24 (m, 2H), 7.04-6.98 | |
| | (m, 1H), 4.61-4.49 (m, 1H), 4.50-3.90 (m, 4H), 3.97 (s, 3H), 2.90 (s, 3H), 2.46-2.31 (m, 4H), | |
| | 2.19 (s, 3H). | |
| Example 105 | 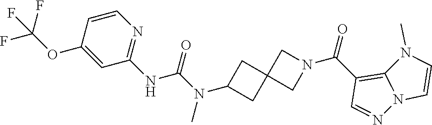 [SMILES: CN(C(=O)Nc1cc(OC(F)(F)F)ccn1)C1CC2(C1)CN(C(=O)c1cnn3ccn(C)c13)C2] | 32 mg (47%) |
| | LCMS (ESI): Method 2, t_{R} = 3.79 min, m/z (M + 1) = 478.4 ¹H NMR (400 MHz, DMSO-d6) δ | |
| | 9.31 (s, 1H), 8.34 (d, J = 5.6 Hz, 1H), 7.84 (s, 1H), 7.77 (s, 1H), 7.67 (d, J = 2.2 Hz, 1H), 7.26 | |
| | (s, 1H), 6.99 (d, J = 5.6 Hz, 1H), 4.64-4.52 (m, 1H), 4.50-3.91 (m, 4H), 3.97 (s, 3H), 2.89 (s, | |
| | 3H), 2.45-2.30 (m, 4H). | |
| Example 107 | 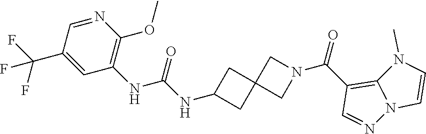 [SMILES: COc1ncc(C(F)(F)F)cc1NC(=O)NC1CC2(C1)CN(C(=O)c1cnn3ccn(C)c13)C2] | 52 mg (40%) |
| | LCMS (ESI): Method 2, t_{R} = 4.06 min, m/z (M + 1) = 478.4 ¹H NMR (400 MHz, DMSO-d6) δ | |
| | 8.66 (d, J = 2.3 Hz, 1H), 8.31 (s, 1H), 8.10 (s, 1H), 7.77 (s, 1H), 7.67 (d, J = 2.2 Hz, 1H), 7.32 | |
| | (d, J = 7.3 Hz, 1H), 7.26 (s, 1H), 4.50-3.80 (m, 5H), 4.02 (s, 3H), 3.97 (s, 3H), 2.61-2.46 (m, | |
| | 2H), 2.11-2.02 (m, 2H). | |
| Example 108 | 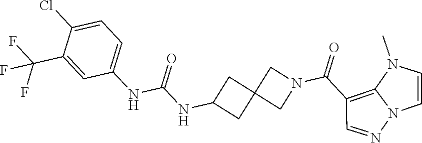 [SMILES: Cn1ccn2ncc(C(=O)N3CC4(CC(NC(=O)Nc5ccc(Cl)c(C(F)(F)F)c5)C4)C3)c12] | 66 mg (35%) |
| | LCMS (ESI): Method 2, t_{R} = 4.29 min, m/z (M + 1) = 481.3 ¹H NMR (400 MHz, DMSO-d6) δ | |
| | 8.89 (s, 1H), 8.04 (d, J = 2.3 Hz, 1H), 7.77 (s, 1H), 7.66 (d, J = 2.2 Hz, 1H), 7.60-7.51 (m, 3H), | |
| | 7.25 (s, 1H), 6.65 (d, J = 7.5 Hz, 1H), 4.53-3.80 (m, 5H), 3.96 (s, 3H), 2.59-2.42 (m, 2H, partly | |
| | covered by residual DMSO signal), 2.20-2.09 (m, 2H). | |
| Example 109 | 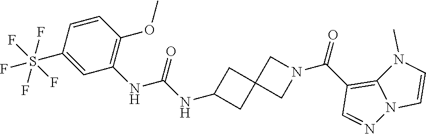 [SMILES: COc1ccc(S(F)(F)(F)(F)F)cc1NC(=O)NC1CC2(C1)CN(C(=O)c1cnn3ccn(C)c13)C2] | 99 mg (48%) |
| | LCMS (ESI): Method 2, t_{R} = 4.33 min, m/z (M + 1) = 535.3 ¹H NMR (400 MHz, DMSO-d6) δ | |
| | 8.74 (d, J = 2.9Hz, 1H), 8.15 (s, 1H), 7.78 (d, J = 0.96 Hz, 1H), 7.67 (d, J = 2.2 Hz, 1H), 7.41 | |
| | (dd, J = 2.9, 9.0 Hz, 1H), 7.30 (d, J = 7.2 Hz, 1H), 7.26 (dd, J = 1.2, 2.0 Hz, 1H), 7.13 (d, J = 9.0 | |
| | Hz, 1H), 4.48-3.89 (m, 5H), 3.97 (s, 3H), 3.94 (s, 3H), 2.59-2.46 (m, 2H,), 2.09-2.01 (m, 2H). | |
| Example 110 | 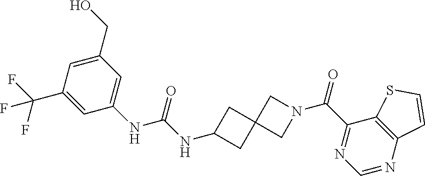 | 42 mg (39%) |
| | LCMS (ESI): Method 2, t_{R} = 3.96 min, m/z (M + 1) = 492.3 ¹H NMR (400 MHz, DMSO-d6) δ | |
| | 9.31-9.28 (m, 1H), 8.77 (d, J = 2.1 Hz, 1H), 8.60 (d, J = 5.6 Hz, 1H), 7.83 (s, 1H), 7.69 (dd, J = | |
| | 2.1, 5.6 Hz, 1H), 7.46 (s, 1H), 7.17 (s, 1H), 6.56 (d, J = 7.6 Hz, 1H), 5.36 (dd, J = 5.8, 5.8 Hz, | |
| | 1H), 4.87 (s, 1H), 4.77 (s, 1H), 4.52 (d, J = 5.8 Hz, 2H), 4.26 (s, 1H), 4.15 (s, 1H), 4.10-4.01 | |
| | (m, 1H), 2.68-2.55 (m, 2H), 2.21-2.14 (m, 2H). | |
| Example 111 | 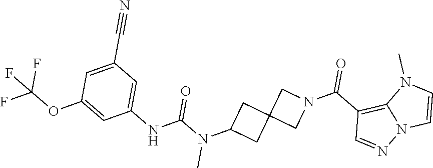 [SMILES: CN(C(=O)Nc1cc(C#N)cc(OC(F)(F)F)c1)C1CC2(C1)CN(C(=O)c1cnn3ccn(C)c13)C2] | 34 mg (26%) |
| | LCMS (ESI): Method 2, t_{R} = 4.27 min, m/z (M + 1) = 502.4 ¹H NMR (400 MHz, DMSO-d6) δ | |
| | 8.87 (s, 1H), 7.93 (d, J = 6.5 Hz, 2H), 7.77 (s, 1H), 7.66 (d, J = 2.1 Hz, 1H), 7.51 (s, 1H), 7.27- | |
| | 7.26 (m, 1H), 4.58-4.49 (m, 1H), 4.38-4.02 (m, 4H), 3.96 (s, 3H), 2.90 (s, 3H), 2.54 (s, 1H), | |
| | 2.44-2.32 (m, 4H). | |
| Example 114 | 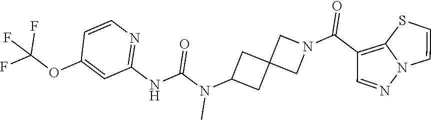 [SMILES: CN(C(=O)Nc1cc(OC(F)(F)F)ccn1)C1CC2(C1)CN(C(=O)c1cnn3ccsc13)C2] | 15 mg (24%) |
| | LCMS (ESI): Method 2, t_{R} = 3.98 min, m/z (M + 1) = 481.4 ¹H NMR (400 MHz, DMSO-d6) δ | |
| | 9.32 (s, 1H), 8.36-8.31 (m, 2H), 8.07 (s, 1H), 7.84 (s, 1H), 7.53 (d, J = 4.0 Hz, 1H), 6.99 (d, J = | |
| | 5.6 Hz, 1H), 4.63-4.54 (m, 2H), 4.41 (s, 1H), 4.11 (s, 1H), 3.98 (s, 1H), 2.90 (s, 3H), 2.47-2.33 | |
| | (m, 4H). | |
| Example 117 | 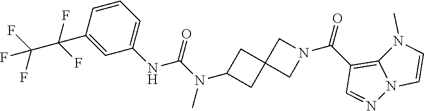 [SMILES: CN(C(=O)Nc1cccc(C(F)(F)C(F)(F)F)c1)C1CC2(C1)CN(C(=O)c1cnn3ccn(C)c13)C2] | 48 mg (37%) |
| | LCMS (ESI): Method 2, t_{R} = 4.47 min, m/z (M + 1) = 511.4 ¹H NMR (400 MHz, DMSO-d6) δ | |
| | 8.61 (s, 1H), 7.89 (s, 1H), 7.81-7.73 (m, 2H), 7.66 (d, J = 2.2 Hz, 1H), 7.48 (t, J = 8.0 Hz, 1H), | |
| | 7.28-7.20 (m, 2H), 4.64-3.98 (m, 5H), 3.96 (s, 3H), 2.89 (s, 3H), 2.44-2.34 (m, 4H). | |
| Example 118 | 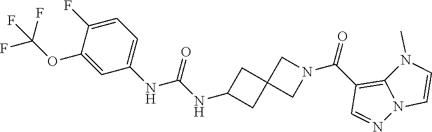 [SMILES: Cn1ccn2ncc(C(=O)N3CC4(CC(NC(=O)Nc5ccc(F)c(OC(F)(F)F)c5)C4)C3)c12] | 50 mg (38%) |
| | LCMS (ESI): Method 2, t_{R} = 4.14 min, m/z (M + 1) = 481.4 ¹H NMR (400 MHz, DMSO-d6) δ | |
| | 8.69 (s, 1H), 7.81-7.76 (m, 2H), 7.67 (d, J = 2.2 Hz, 1H), 7.36 (dd, J = 9.0, 10.0 Hz, 1H), 7.28- | |
| | 7.21 (m, 2H), 6.55 (d, J = 7.6 Hz, 1H), 4.56-3.82 (m, 5H), 3.97 (s, 3H), 2.57-2.50 (m, 2H), | |
| | 2.18-2.08 (m, 2H). | |
| Example 119 | 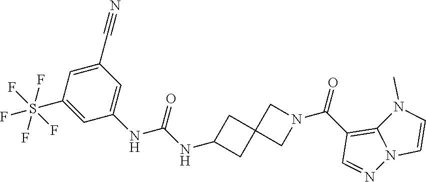 [SMILES: Cn1ccn2ncc(C(=O)N3CC4(CC(NC(=O)Nc5cc(C#N)cc(S(F)(F)(F)(F)F)c5)C4)C3)c12] | 50 mg (41%) |
| | LCMS (ESI): Method 2, t_{R} = 4.26 min, m/z (M + 1) = 530.4 ¹H NMR (400 MHz, DMSO-d6) δ | |
| | 9.21 (s, 1H), 8.39 (t, J = 1.8 Hz, 1H), 8.01 (s, 2H), 7.77 (s, 1H), 7.66 (d, J = 2.4 Hz, 1H), 7.25 | |
| | 6.96 (d, J = 6.8 Hz, 1H), (s, 1H), 4.20-3.95 (m, 8H), 2.20-2.12 (m, 2H). | |
| Example 120 | 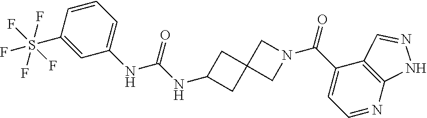 [SMILES: O=C(Nc1cccc(S(F)(F)(F)(F)F)c1)NC1CC2(C1)CN(C(=O)c1ccnc3[nH]ncc13)C2] | 42 mg (56%) |
| | LCMS (ESI): Method 2, t_{R} = 4.08 min, m/z (M + 1) = 503.2 ¹H NMR (400 MHz, DMSO-d6) δ | |
| | 13.88 (s, 1H), 8.84 (d, J = 13.1 Hz, 1H), 8.62 (t, J = 5.0 Hz, 1H), 8.24 (s, 1H), 8.17 (s, 1H), | |
| | 7.50-7.36 (m, 3H), 7.33 (t, J = 5.0 Hz, 1H), 6.59 (dd, J = 4.7, 7.0 Hz, 1H), 4.30 (d, J = 45.0 | |
| | Hz, 2H), 4.16 (d, J = 45.0 Hz, 2H), 4.10-3.93 (m, 1H), 2.60-2.44 (m, 2H), 2.20-2.06 (m, 2H). | |
| Example 121 | 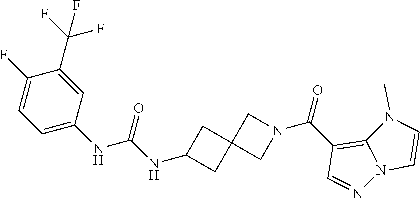 [SMILES: Cn1ccn2ncc(C(=O)N3CC4(CC(NC(=O)Nc5ccc(F)c(C(F)(F)F)c5)C4)C3)c12] | 57 mg (45%) |
| | LCMS (ESI): Method 2, t_{R} = 4.03 min, m/z (M + 1) = 465.4 ¹H NMR (400 MHz, DMSO-d6) δ | |
| | 8.73 (s, 1H), 7.93 (dd, J = 2.6, 6.2 Hz, 1H), 7.77 (s, 1H), 7.66 (d, J = 2.2 Hz, 1H), 7.60-7.53 (m, | |
| | 1H), 7.36 (t, J = 9.8 Hz, 1H), 7.25 (s, 1H), 6.58 (d, J = 7.9 Hz, 1H), 4.58-3.57 (m, 5H), 3.96 (s, | |
| | 3H), 2.56-2.43 (m, 2H), 2.17-2.09 (m, 2H). | |
| Example 123 | 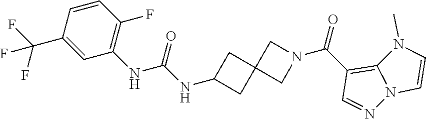 [SMILES: Cn1ccn2ncc(C(=O)N3CC4(CC(NC(=O)Nc5cc(C(F)(F)F)ccc5F)C4)C3)c12] | 97 mg (91%) |
| | LCMS (ESI): Method 2, t_{R} = 4.09 min, m/z (M + 1) = 465.2 ¹H NMR (400 MHz, DMSO-d6) δ | |
| | 8.59-8.54 (m, 2H), 7.77 (s, 1H), 7.66 (d, J = 2.1 Hz, 1H), 7.46-7.40 (m, 1H), 7.33-7.25 (m, 2H), | |
| | 7.01 (d, J = 7.3 Hz, 1H), 4.19-3.96 (m, 8H), 2.59-2.52 (m, 2H), 2.12-2.05 (m, 2H). | |
| Example 124 | 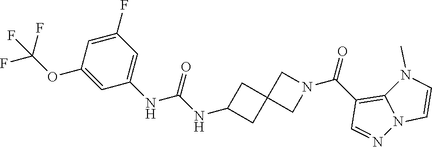 [SMILES: Cn1ccn2ncc(C(=O)N3CC4(CC(NC(=O)Nc5cc(F)cc(OC(F)(F)F)c5)C4)C3)c12] | 70 mg (63%) |
| | LCMS (ESI): Method 2, t_{R} = 4.3 min, m/z (M + 1) = 481 ¹H NMR (400 MHz, DMSO-d6) δ | |
| | 8.91 (s, 1H), 7.77 (s, 1H), 7.66 (d, J = 2.1 Hz, 1H), 7.33-7.25 (m, 3H), 6.82 (d, J = 8.9 Hz, | |
| | 1H), 6.67 (d, J = 7.3 Hz, 1H), 4.18-3.96 (m, 8H), 2.15-2.10 (m, 2H). | |
| Example 125 | 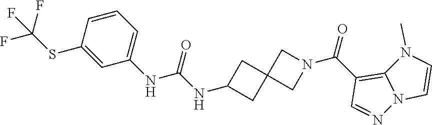 [SMILES: Cn1ccn2ncc(C(=O)N3CC4(CC(NC(=O)Nc5cccc(SC(F)(F)F)c5)C4)C3)c12] | 52 mg (40%) |
| | LCMS (ESI): Method 2, t_{R} = 4.27 min, m/z (M + 1) = 479.3 ¹H NMR (400 MHz, DMSO-d6) δ | |
| | 8.71 (s, 1H), 7.91 (br s, 1H), 7.78 (d, J = 0.9 Hz, 1H), 6.67 (d, J = 2.2 Hz, 1H), 7.52-7.47 (m, | |
| | 1H), 7.38 (t, J = 7.9 Hz, 1H), 7.28-7.25 (m, 1H), 7.25-7.20 (m, 1H), 6.56 (d, J = 7.6 Hz, 1H), | |
| | 4.52-3.73 (m, 5H), 3.97 (s, 3H), 2.58-2.49 (m, 2H), 2.18-2.2.08 (m, 2H). | |
| Example 126 | 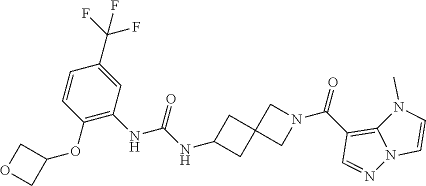 [SMILES: Cn1ccn2ncc(C(=O)N3CC4(CC(NC(=O)Nc5cc(C(F)(F)F)ccc5OC5COC5)C4)C3)c12] | 22 mg (16%) |
| | LCMS (ESI): Method 2, t_{R} = 3.95 min, m/z (M + 1) = 519.4 ¹H NMR (400 MHz, DMSO-d6) δ | |
| | 8.53 (d, J = 2.2 Hz, 1H), 8.07 (s, 1H), 7.78 (d, J = 0.88 Hz, 1H), 7.67 (d, J = 2.2 Hz, 1H), 7.34 (d, | |
| | J = 7.2 Hz, 1H), 7.29-7.23 (m, 1H), 7.18 (dd, J = 1.8, 8.5 Hz, 1H), 6.77 (d, J = 8.5 Hz, 1H), 5.42 | |
| | (p, J = 5.6 Hz, 1H), 4.99 (t, J = 6.7 Hz, 2H), 4.65 (dd, J = 4.9, 7.6 Hz, 2H), 4.56-3.83 (m, 5H), | |
| | 3.97 (s, 3H), 2.64-2.46 (m, 2H, partly covered by residual DMSO peak), 2.16-2.03 (m, 2H). | |
| Example 127 | 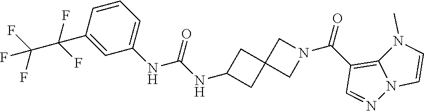 [SMILES: Cn1ccn2ncc(C(=O)N3CC4(CC(NC(=O)Nc5cccc(C(F)(F)C(F)(F)F)c5)C4)C3)c12] | 40 mg (30%) |
| | LCMS (ESI): Method 2, t_{R} = 4.32 min, m/z (M + 1) = 497.4 ¹H NMR (400 MHz, DMSO-d6) δ | |
| | 8.79 (s, 1H), 7.93 (s, 1H), 7.78 (d, J = 0.85 Hz, 1H), 7.67 (d, J = 2.2 Hz, 1H), 7.54 (d, J = 8.6 | |
| | Hz, 1H), 7.47 (t, J = 7.9 Hz, 1H), 7.28-7.24 (m, 1H), 7.19 (d, J = 7.7 Hz, 1H), 6.57 (d, J = 7.6 | |
| | Hz, 1H), 4.54-3.81 (m, 5H), 3.97 (s, 3H), 2.59-2.45 (m, 2H), 2.19-2.09 (m, 2H) | |
| Example 129 | 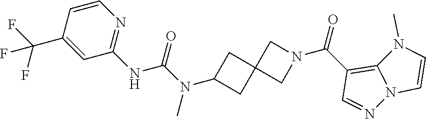 [SMILES: CN(C(=O)Nc1cc(C(F)(F)F)ccn1)C1CC2(C1)CN(C(=O)c1cnn3ccn(C)c13)C2] | 52 mg (48%) |
| | LCMS (ESI): Method 2, t_{R} = 2.84 min, m/z (M + 1) = 462.1 ¹H NMR (400 MHz, DMSO-d6) δ | |
| | 9.42 (s, 1H), 8.50 (d, J = 5.3 Hz, 1H), 8.13 (s, 1H), 7.76 (s, 1H), 7.66 (d, J = 2.1 Hz, 1H), 7.30 | |
| | (dd, J = 0.9, 4.3 Hz, 1H), 7.26-7.25 (m, 1H), 4.62-4.52 (m, 1H), 4.18-4.17 (m, 6H), 3.96 (s, 3H), | |
| | 2.90 (s, 3H), 2.44-2.32 (m, 4H). | |
| Example 131 | 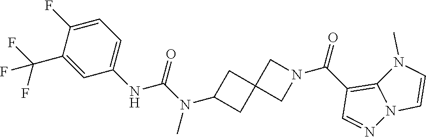 [SMILES: CN(C(=O)Nc1ccc(F)c(C(F)(F)F)c1)C1CC2(C1)CN(C(=O)c1cnn3ccn(C)c13)C2] | 35 mg (37%) |
| | LCMS (ESI): Method 2, t_{R} = 4.16 min, m/z (M + 1) = 479.4 ¹H NMR (400 MHz, DMSO-d6) δ | |
| | 8.60 (s, 1H), 7.91 (dd, J = 2.7, 6.6 Hz, 1H), 7.81-7.78 (m, 1H), 7.77 (s, 1H), 7.66 (d, J = 2.1 Hz, | |
| | 1H), 7.37 (t, J = 9.6 Hz, 1H), 7.26 (s, 1H), 4.60-4.50 (m, 1H), 4.43-4.02 (m, 4H), 3.96 (s, 3H), | |
| | 2.88 (s, 3H), 2.42-2.37 (m, 4H). | |
| Example 132 | 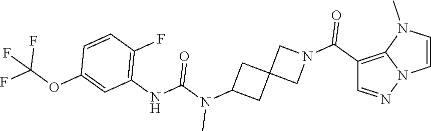 [SMILES: CN(C(=O)Nc1cc(OC(F)(F)F)ccc1F)C1CC2(C1)CN(C(=O)c1cnn3ccn(C)c13)C2] | 45 mg (45%) |
| | LCMS (ESI): Method 2, t_{R} = 4.28 min, m/z (M + 1) = 495.4 ¹H NMR (400 MHz, DMSO-d6) δ | |
| | 8.55 (s, 1H), 7.79-7.76 (m, 2H), 7.67 (d, J = 2.3 Hz, 1H), 7.50-7.46 (m, 1H), 7.3 (t, J = 9.3 Hz, | |
| | 1H), 7.27-7.26 (m, 1H), 4.60-4.51 (m, 1H), 4.44-4.02 (m, 4H), 3.97 (s, 3H), 2.88 (s, 3H), 2.43- | |
| | 2.33 (m, 4H). | |
| Example 145 | 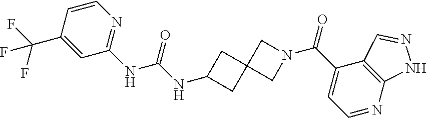 [SMILES: O=C(Nc1cc(C(F)(F)F)ccn1)NC1CC2(C1)CN(C(=O)c1ccnc3[nH]ncc13)C2] | 27 mg (40%) |
| | LCMS (ESI): Method 2, t_{R} = 3.61 min, m/z (M + 1) = 446.4 ¹H NMR (400 MHz, DMSO-d6) δ | |
| | 13.87 (s, 1H), 9.42 (d, J = 12.2 Hz, 1H), 8.61 (t, J = 5.0 Hz, 1H), 8.44 (dd, J = 5.2, 9.4 Hz, 1H), | |
| | 8.24 (s, 1H), 7.92 (d, J = 2.8 Hz, 1H), 7.61 (dd, J = 7.0, 17.3 Hz, 1H), 7.32 (dd, J = 4.8, 5.9 Hz, | |
| | 1H), 7.26 (br s, 1H), 4.38-3.93 (m, 5H), 2.63-2.53 (m, 2H), 2.21-2.08 (m, 2H). | |
| Example 150 | 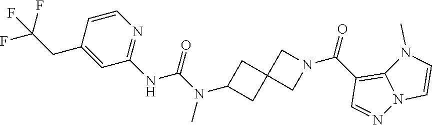 [SMILES: CN(C(=O)Nc1cc(CC(F)(F)F)ccn1)C1CC2(C1)CN(C(=O)c1cnn3ccn(C)c13)C2] | 8.3 mg (19%) |
| | LCMS (ESI): Method 2, t_{R} = 3.08 min, m/z (M + 1) = 476.6 ¹H NMR (400 MHz, DMSO-d6) δ | |
| | 8.94 (s, 1H), 8.22 (d, J = 5.1 Hz, 1H), 7.84 (s, 1H), 7.76 (s, 1H), 7.66 (d, J = 2.0 Hz, 1H), 7.26 | |
| | (s, 1H), 6.97 (d, J = 5.0 Hz, 1H), 4.62-3.86 (m, 5H), 3.96 (s, 3H), 3.69 (q, J = 11.5 Hz, 2H), 2.88 | |
| | (s, 3H), 2.45-2.29 (m, 4H). | |
| Example 151 | 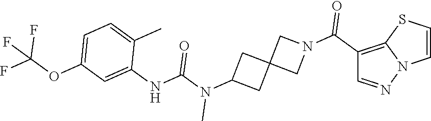 [SMILES: Cc1ccc(OC(F)(F)F)cc1NC(=O)N(C)C1CC2(C1)CN(C(=O)c1cnn3ccsc13)C2] | 80 mg (44%) |
| | LCMS (ESI): Method 2, t_{R} = 4.41 min, m/z (M + 1) = 494.4 ¹H NMR (400 MHz, DMSO-d6) δ | |
| | 8.31 (d, J = 4.23 Hz, 1H), 8.06 (s, 1H), 7.83 (s, 1H), 7.52 (d, J = 4.23 Hz, 1H), 7.35 (s, 1H), 7.28 | |
| | (d, J = 7.81 Hz, 1H), 7.00 (d, J = 8.14 Hz, 1H), 4.60-4.51 (m, 2H), 4.41 (s, 1H), 4.10 (s, 1H), | |
| | 3.98 (s, 1H), 2.89 (s, 3H), 2.46-2.36 (m, 4H), 2.18 (s, 3H). | |
| Example 152 | 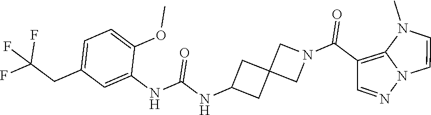 [SMILES: COc1ccc(CC(F)(F)F)cc1NC(=O)NC1CC2(C1)CN(C(=O)c1cnn3ccn(C)c13)C2] | 50 mg (38%) |
| | LCMS (ESI): Method 2, t_{R} = 4.03 min, m/z (M + 1) = 491.6 ¹H NMR (400 MHz, DMSO-d6) δ | |
| | 8.1 (s, 1H), 7.82 (s, 1H), 7.77 (s, 1H), 7.66 (s, 1H), 7.25 (s, 1H), 7.16 (d, J = 7.3 Hz, 1H), 6.95 | |
| | (d, J = 8.4 Hz, 1H), 6.84 (d, J = 8.4 Hz, 1H), 4.50-3.89 (m, 5H), 3.96 (s, 3H), 3.83 (s, 3H), | |
| | 3.47 (q, J = 11.6 Hz, 2H), 2.58-2.45 (m, 2H, partly covered by residual DMSO peak), 2.10- | |
| | 1.98 (m, 2H). | |
| Example 155 | 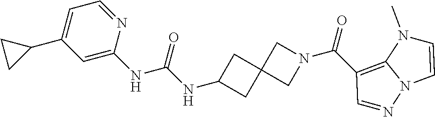 [SMILES: Cn1ccn2ncc(C(=O)N3CC4(CC(NC(=O)Nc5cc(C6CC6)ccn5)C4)C3)c12] | 36 mg (31%) |
| | LCMS (ESI): Method 2, t_{R} = 2.63 min, m/z (M + 1) = 420.8 ¹H NMR (400 MHz, DMSO-d6) δ | |
| | 9.00 (s, 1H), 8.46 (d, J = 6.8 Hz, 1H), 8.00 (d, J = 5.4 Hz, 1H), 7.76 (s, 1H), 7.66 (d, J = 2.2 Hz, | |
| | 1H), 7.25 (s, 1H), 7.04 (s, 1H), 6.62 (dd, J = 1.2, 5.4 Hz, 1H), 4.61-3.85 (m, 5H), 3.96 (s, 3H), | |
| | 2.60-2.36 (m, 2H), 2.17-2.06 (m, 2H), 1.90-1.78 (m, 1H), 1.09-0.97 (m, 2H), 0.76-0.66 (m, 2H). | |
| Example 156 | 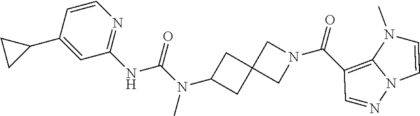 [SMILES: CN(C(=O)Nc1cc(C2CC2)ccn1)C1CC2(C1)CN(C(=O)c1cnn3ccn(C)c13)C2] | 8 mg (7%) |
| | LCMS (ESI): Method 2, t_{R} = 2.61 min, m/z (M + 1) = 434.8 ¹H NMR (400 MHz, DMSO-d6) δ | |
| | 8.68 (s, 1H), 8.03 (d, J = 5.2 Hz, 1H), 7.77 (s, 1H), 7.67 (d, J = 2.2 Hz, 1H), 7.54 (s, 1H), 7.26 | |
| | (s, 1H), 6.67 (dd, J = 1.4, 5.2 Hz), 4.63-4.50 (m, 1H), 4.50-3.83 (m, 4H), 3.97 (s, 3H), 2.87 (s, | |
| | 3H), 2.47-2.28 (m, 4H), 1.94-1.82 (m, 1H), 1.08-0.98 (m, 2H), 0.77-0.68 (m, 2H). | |
| Example 157 | 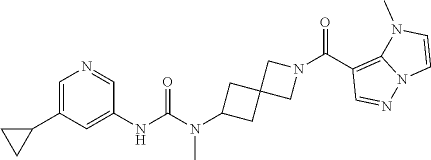 [SMILES: CN(C(=O)Nc1cncc(C2CC2)c1)C1CC2(C1)CN(C(=O)c1cnn3ccn(C)c13)C2] | 24 mg (22%) |
| | LCMS (ESI): Method 2, t_{R} = 2.52 min, m/z (M + 1) = 434.7 ¹H NMR (400 MHz, DMSO-d6) δ | |
| | 8.43 (d, J = 2.3 Hz, 1H), 8.36 (s, 1H), 8.00 (d, J = 1.9 Hz, 1H), 7.77 (s, 1H), 7.67 (d, J = 2.2 Hz, | |
| | 1H), 7.47 (t, J = 2.1 Hz, 1H), 7.26 (s, 1H), 4.64-4.50 (m, 1H), 4.50-3.76 (m, 4H), 3.97 (s, 3H), | |
| | 2.89 (s, 3H), 2.45-2.30 (m, 4H), 1.95-1.86 (m, 1H), 1.03-0.93 (m, 2H), 0.69-0.62 (m, 2H). | |
| Example 158 | 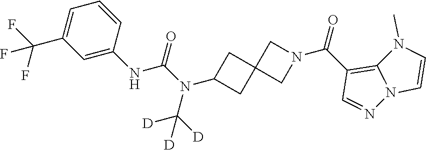 [SMILES: [2H]C([2H])([2H])N(C(=O)Nc1cccc(C(F)(F)F)c1)C1CC2(C1)CN(C(=O)c1cnn3ccn(C)c13)C2] | 71 mg (60%) |
| | LCMS (ESI): Method 2, t_{R} = 4.11 min, m/z (M + 1) = 464.5 ¹H NMR (400 MHz, DMSO-d6) δ | |
| | 8.59 (s, 1H), 7.91 (s, 1H), 7.98-7.71 (m, 2H), 7.66 (d, J = 2.2 Hz, 1H), 7.46 (t, J = 7.9 Hz, 1H), | |
| | 7.28-7.23 (m, 2H), 4.61-4.50 (m, 1H), 4.51-3.78 (m, 4H), 3.96 (s, 3H), 2.45-2.30 (m, 4H). | |
| Example 159 | 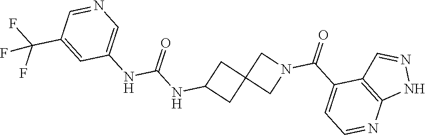 [SMILES: O=C(Nc1cncc(C(F)(F)F)c1)NC1CC2(C1)CN(C(=O)c1ccnc3[nH]ncc13)C2] | 55 mg (52%) |
| | LCMS (ESI): Method 2, t_{R} = 3.24 min, m/z (M + 1) = 446.5 ¹H NMR (400 MHz, DMSO-d6) δ | |
| | 13.87 (s, 1H), 8.98 (d, J = 14.3 Hz, 1H), 8.69 (s, 1H), 8.61 (t, J = 4.8 Hz, 1H), 8.47 (s, 1H), 8.35 | |
| | (t, J = 2.4 Hz, 1H), 8.23 (s, 1H), 7.32 (t, J = 4.7 Hz, 1H), 6.81 (m, 1H), 4.35 (s, 1H), 4.24 (s, | |
| | 1H), 4.20 (s, 1H), 4.10 (s, 1H), 4.08-3.94 (m, 1H), 2.56-2.53 (m, 2H), 2.21-2.07 (m, 2H). | |
| Example 161 | 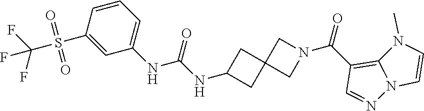 [SMILES: Cn1ccn2ncc(C(=O)N3CC4(CC(NC(=O)Nc5cccc(S(=O)(=O)C(F)(F)F)c5)C4)C3)c12] | 82 mg (52%) |
| | LCMS (ESI): Method 2, t_{R} = 4.05 min, m/z (M + 1) = 511.5 ¹H NMR (400 MHz, DMSO-d6) δ | |
| | 9.09 (s, 1H), 8.41 (s, 1H), 7.84-7.76 (m, 2H), 7.70 (d, J = 7.8 Hz, 1H), 7.67 (d, J = 2.4 Hz, 1H), | |
| | 7.62 (d, J = 8.0 Hz, 1H), 7.26 (s, 1H), 6.71 (d, J = 7.4 Hz, 1H), 4.57-3.75 (m, 5H), 3.97 (s, 3H), | |
| | 2.62-2.42 (m, 2H), 2.22-2.10 (m, 2H). | |
| Example 162 | 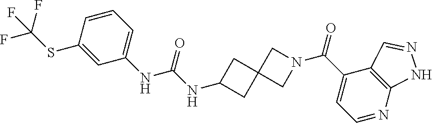 [SMILES: O=C(Nc1cccc(SC(F)(F)F)c1)NC1CC2(C1)CN(C(=O)c1ccnc3[nH]ncc13)C2] | 86 mg (67%) |
| | LCMS (ESI): Method 2, t_{R} = 4.15 min, m/z (M + 1) = 477.4 ¹H NMR (400 MHz, DMSO-d6) δ | |
| | 13.88 (s, 1H), 8.70 (d, J = 12.7 Hz, 1H), 8.61 (t, J = 5.1 Hz, 1H), 8.24 (s, 1H), 7.90 (s, 1H), 7.47 | |
| | (d, J = 8.5 Hz, 1H), 7.37 (t, J = 7.9 Hz, 1H), 7.32 (t, J = 5.0 Hz, 1H), 7.22 (d, J = 7.5 Hz, 1H), | |
| | 6.54 (t, J = 6.2 Hz, 1H), 4.40-3.90 (m, 5H), 2.62-2.43 (m, 2H), 2.22-2.04 (m, 2H). | |
| Example 164 | 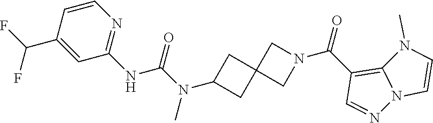 [SMILES: CN(C(=O)Nc1cc(C(F)F)ccn1)C1CC2(C1)CN(C(=O)c1cnn3ccn(C)c13)C2] | 43 mg (44%) |
| | LCMS (ESI): Method 2, t_{R} = 3.25 min, m/z (M + 1) = 444.3 ¹H NMR (400 MHz, DMSO-d6) δ | |
| | 9.19 (s, 1H), 8.39 (d, J = 5.0 Hz, 1H), 8.00 (s, 1H), 7.77 (s, 1H), 7.67 (d, J = 2.3 Hz, 1H), 7.26- | |
| | 7.26 (m, 1H), 7.13 (d, J = 4.9 Hz, 1H), 7.06 (t, J = 55.1 Hz, 1H), 4.62-4.52 (m, 1H), 4.46-4.02 | |
| | (m, 4H), 3.97 (s, 3H), 2.90 (s, 3H), 2.45-2.32 (m, 4H). | |
| Example 165 | 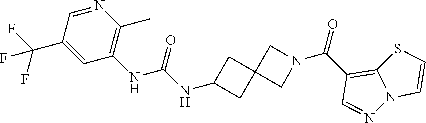 [SMILES: Cc1ncc(C(F)(F)F)cc1NC(=O)NC1CC2(C1)CN(C(=O)c1cnn3ccsc13)C2] | 42 mg (32%) |
| | LCMS (ESI): Method 2, t_{R} = 3.58 min, m/z (M + 1) = 465.4 ¹H NMR (400 MHz, DMSO-d6) δ | |
| | 8.65 (d, J = 1.9 Hz, 1H), 8.39 (s, 1H), 8.32 (d, J = 4.1 Hz, 1H), 8.08 (d, J = 3.1 Hz, 2H), 7.53 | |
| | (dd, J = 1.4, 4.1 Hz, 1H), 7.16 (d, J = 7.3 Hz, 1H), 4.53 (1, 1H), 4.42 (s, 1H), 4.15-4.00 (m, 3H), | |
| | 2.61-2.56 (m, 2H), 2.18-2.11 (m, 2H). | |
| Example 168 | 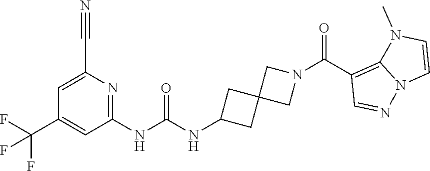 [SMILES: Cn1ccn2ncc(C(=O)N3CC4(CC(NC(=O)Nc5cc(C(F)(F)F)cc(C#N)n5)C4)C3)c12] | 10 mg (8%) |
| | LCMS (ESI): Method 2, t_{R} = 3.97 min, m/z (M + 1) = 473.4 ¹H NMR (400 MHz, DMSO-d6) δ | |
| | 9.89 (s, 1H), 8.43 (s, 1H), 8.06 (s, 1H), 7.77 (s, 1H), 7.66 (d, J = 2.3 Hz, 1H), 7.26 (s, 1H), 7.11 | |
| | (d, J = 7.5 Hz, 1H), 4.45-4.04 (m, 4H), 3.96 (s, 3H), 2.59-2.53 (m, 2H), 2.18-2.11 (m, 2H). | |
| Example 169 | 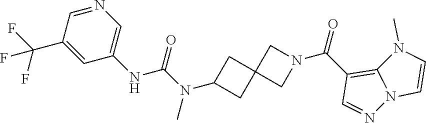 [SMILES: CN(C(=O)Nc1cncc(C(F)(F)F)c1)C1CC2(C1)CN(C(=O)c1cnn3ccn(C)c13)C2] | 58 mg (57%) |
| | LCMS (ESI): Method 2, t_{R} = 3.52 min, m/z (M + 1) = 462.3 ¹H NMR (400 MHz, DMSO-d6) δ | |
| | 8.94 (d, J = 2.0 Hz, 1H), 8.84 (s, 1H), 8.51 (s, 1H), 8.30 (dd, J = 2.4, 2.4 Hz, 1H), 7.77 (s, 1H), | |
| | 7.66 (d, J = 2.3 Hz, 1H), 7.27-7.25 (m, 1H), 4.61-4.52 (m, 1H), 4.45-4.02 (m, 4H), 3.96 (s, 3H), | |
| | 2.92 (s, 3H), 2.44-2.37 (m, 4H) | |
| Example 170 | 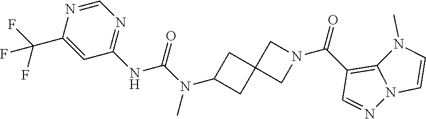 [SMILES: CN(C(=O)Nc1cc(C(F)(F)F)ncn1)C1CC2(C1)CN(C(=O)c1cnn3ccn(C)c13)C2] | 72 mg (61%) |
| | LCMS (ESI): Method 2, t_{R} = 3.57 min, m/z (M + 1) = 463.4 ¹H NMR (400 MHz, DMSO-d6) δ | |
| | 10.20 (s, 1H), 8.96 (s, 1H), 8.15 (s, 1H), 7.76 (s, 1H), 7.66 (d, J = 2.2 Hz, 1H), 7.26 (s, 1H), | |
| | 4.61-3.97 (m, 5H), 3.96 (s, 3H), 2.91 (s, 3H), 2.48-2.30 (m, 4H, partly covered by residual | |
| | DMSO peak). | |
| Example 171 | 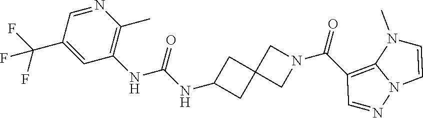 [SMILES: Cc1ncc(C(F)(F)F)cc1NC(=O)NC1CC2(C1)CN(C(=O)c1cnn3ccn(C)c13)C2] | 1.8 mg (3.6%) |
| | LCMS (ESI): Method 2, t_{R} = 3.44 min, m/z (M + 1) = 462.4 ¹H NMR (400 MHz, DMSO-d6) δ | |
| | 8.64 (d, J = 1.9 Hz, 1H), 8.39 (s, 1H), 8.08 (s, 1H), 7.78 (s, 1H), 7.67 (d, J = 2.1 Hz, 1H), 7.27- | |
| | 7.26 (m, 1H), 7.16 (d, J = 7.4 Hz, 1H), 4.47-3.97 (m, 8H), 2.59-2.54 (m, 2H), 2.15-2.07 (m, 2H). | |
| Example 172 | 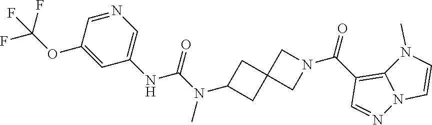 [SMILES: CN(C(=O)Nc1cncc(OC(F)(F)F)c1)C1CC2(C1)CN(C(=O)c1cnn3ccn(C)c13)C2] | 39 mg (44%) |
| | LCMS (ESI): Method 2, t_{R} = 3.64 min, m/z (M + 1) = 478.6 ¹H NMR (400 MHz, DMSO-d6) δ | |
| | 8.81 (s, 1H), 8.70 (d, J = 2.0 Hz, 1H), 8.22 (d, J = 2.3 Hz, 1H), 8.08 (s, 1H), 7.77 (s, 1H), 7.67 | |
| | (d, J = 2.3 Hz, 1H), 7.27-7.26 (m, 1H), 4.62-4.52 (m, 1H), 4.19-4.19 (m, 4H), 3.97 (s, 3H), 2.91 | |
| | (s, 3H), 2.42-2.33 (m, 4H). | |
| Example 173 | 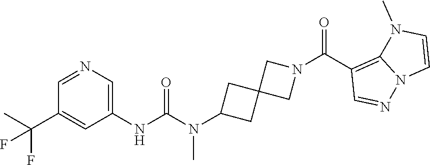 [SMILES: CN(C(=O)Nc1cncc(C(C)(F)F)c1)C1CC2(C1)CN(C(=O)c1cnn3ccn(C)c13)C2] | 27 mg (31%) |
| | LCMS (ESI): Method 2, t_{R} = 3.15 min, m/z (M + 1) = 458.6 ¹H NMR (400 MHz, DMSO-d6) δ | |
| | 8.79 (d, J = 2.3 Hz, 1H), 8.68 (s, 1H), 8.36 (s, 1H), 8.09 (dd, J = 2.4, 2.4 Hz, 1H), 7.77 (s, 1H), | |
| | 7.67 (d, J = 2.3 Hz, 1H), 7.26 (s, 1H), 4.62-4.52 (m, 1H), 4.44-4.03 (m, 4H), 3.97 (s, 3H), 2.91 | |
| | (s, 3H), 2.46-2.37 (m, 4H), 2.00 (dd, J = 19.1, 19.1 Hz, 3H). | |
| Example 175 | 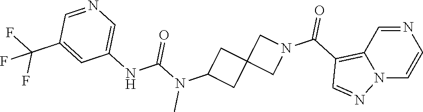 [SMILES: CN(C(=O)Nc1cncc(C(F)(F)F)c1)C1CC2(C1)CN(C(=O)c1cnn3ccncc13)C2] | 35 mg (43%) |
| | LCMS (ESI): Method 2, t_{R} = 3.43 min, m/z (M + 1) = 460.4 ¹H NMR (400 MHz, DMSO-d6) δ | |
| | 9.56 (d, J = 1.5 Hz, 1H), 8.96-8.91 (m, 2H), 8.86 (s, 1H), 8.52 (s, 1H), 8.45 (d, J = 5.6 Hz, 1H), | |
| | 8.31 (t, J = 2.4, 2.4 Hz, 1H), 8.12 (d, J = 4.6 Hz, 1H), 4.65-4.54 (m, 2H), 4.49 (s, 1H), 4.19 (s, | |
| | 1H), 4.06-4.06 (m, 1H), 2.93 (s, 3H), 2.46-2.43 (m, 4H). | |
| Example 176 | 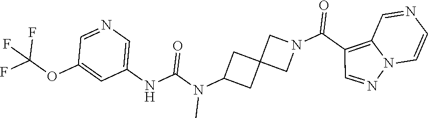 [SMILES: CN(C(=O)Nc1cncc(OC(F)(F)F)c1)C1CC2(C1)CN(C(=O)c1cnn3ccncc13)C2] | 54 mg (66%) |
| | LCMS (ESI): Method 2, t_{R} = 3.54 min, m/z (M + 1) = 476.5 ¹H NMR (400 MHz, DMSO-d6) δ | |
| | 9.56 (d, J = 1.5 Hz, 1H), 8.92 (dd, J = 1.3, 4.7 Hz, 1H), 8.82 (s, 1H), 8.70 (d, J = 2.1 Hz, 1H), | |
| | 8.45 (d, J = 4.6 Hz, 1H), 8.22 (d, J = 2.1 Hz, 1H), 8.12 (d, J = 4.8 Hz, 1H), 8.07 (s, 1H), 4.64- | |
| | 4.54 (m, 2H), 4.49 (s, 1H), 4.20 (s, 1H), 4.06 (s, 1H), 2.92 (s, 3H), 2.44 (m, 4H). | |
| Example 177 | 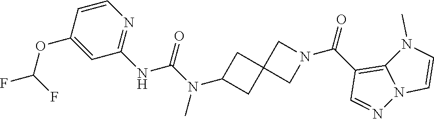 [SMILES: CN(C(=O)Nc1cc(OC(F)F)ccn1)C1CC2(C1)CN(C(=O)c1cnn3ccn(C)c13)C2] | 28 mg (33%) |
| | LCMS (ESI): Method 2, t_{R} = 3.03 min, m/z (M + 1) = 460.7 ¹H NMR (400 MHz, DMSO-d6) δ | |
| | 9.11 (s, 1H), 8.23 (d, J = 5.6 Hz, 1H), 7.77 (s, 1H), 7.66 (d, J = 2.5 Hz, 1H), 7.64 (d, J = 2.2 Hz, | |
| | 1H), 7.41 (t, J = 72.5 Hz, 1H), 7.26-7.26 (m, 1H), 6.81 (dd, J = 2.3, 5.6 Hz, 1H), 4.62-4.52 (m, | |
| | 1H), 4.41-4.07 (m, 4H), 3.97 (s, 3H), 2.89 (s, 3H), 2.45-2.32 (m, 4H). | |
| Example 178 | 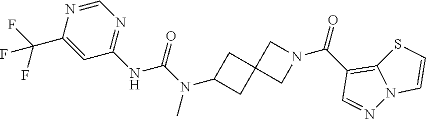 [SMILES: CN(C(=O)Nc1cc(C(F)(F)F)ncn1)C1CC2(C1)CN(C(=O)c1cnn3ccsc13)C2] | 35 mg (49%) |
| | LCMS (ESI): Method 2, t_{R} = 3.72 min, m/z (M + 1) = 466.6 ¹H NMR (400 MHz, DMSO-d6) δ | |
| | 10.21 (s, 1H), 8.96 (s, 1H), 8.32 (d, J = 4.1 Hz, 1H), 8.15 (s, 1H), 8.07 (s, 1H), 7.53 (dd, J = 1.2, | |
| | 4.1 Hz, 1H), 4.61-4.40 (m, 3H), 4.14-3.97 (m, 2H), 2.92 (s, 3H), 2.46-2.32 (m, 4H). | |
| Example 179 | 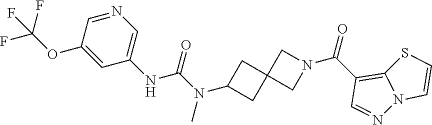 [SMILES: CN(C(=O)Nc1cncc(OC(F)(F)F)c1)C1CC2(C1)CN(C(=O)c1cnn3ccsc13)C2] | 37 mg (51%) |
| | LCMS (ESI): Method 2, t_{R} = 3.78 min, m/z (M + 1) = 481.8 ¹H NMR (400 MHz, DMSO-d6) δ | |
| | 8.82 (s, 1H), 8.70 (d, J = 2.0 Hz, 1H), 8.32 (d, J = 4.1 Hz, 1H), 8.22 (d, J = 2.3 Hz, 1H), 8.07 (s, | |
| | 2H), 7.53 (dd, J = 1.2, 4.1 Hz, 1H), 4.63-4.40 (m, 2H), 4.16-3.97 (m, 2H), 2.92 (s, 3H), 2.46- | |
| | 2.32 (m, 4H). | |
| Example 180 | 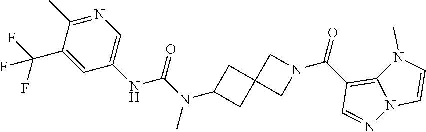 [SMILES: Cc1ncc(NC(=O)N(C)C2CC3(C2)CN(C(=O)c2cnn4ccn(C)c24)C3)cc1C(F)(F)F] | 49 mg (56%) |
| | LCMS (ESI): Method 2, t_{R} = 3.60 min, m/z (M + 1) = 476.7 ¹H NMR (400 MHz, DMSO-d6) δ | |
| | 8.80 (d, J = 2.3 Hz, 1H), 8.72 (s, 1H), 8.24 (d, J = 2.4 Hz, 1H), 7.77 (s, 1H), 7.67 (d, J = 2.3 Hz, | |
| | 1H), 7.26 (s, 1H), 4.62-4.52 (m, 1H), 4.46-4.05 (m, 4H), 3.97 (s, 3H), 2.91 (s, 3H), 2.55 (d, J = | |
| | 1.5 Hz, 3H), 2.44-2.37 (m, 4H). | |
| Example 181 | 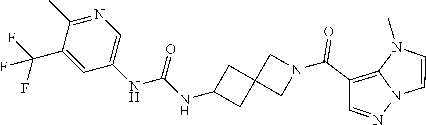 [SMILES: Cc1ncc(NC(=O)NC2CC3(C2)CN(C(=O)c2cnn4ccn(C)c24)C3)cc1C(F)(F)F] | 52 mg (58%) |
| | LCMS (ESI): Method 2, t_{R} = 3.46 min, m/z (M + 1) = 462.6 ¹H NMR (400 MHz, DMSO-d6) δ | |
| | 8.91 (s, 1H), 8.60 (d, J = 2.0 Hz, 1H), 8.29 (d, J = 2.4 Hz, 1H), 7.77 (s, 1H), 7.66 (d, J = 2.1 Hz, | |
| | 1H), 7.26-7.26 (m, 1H), 6.80 (d, J = 7.6 Hz, 1H), 4.38-3.99 (m, 5H), 3.96 (s, 3H), 2.57-2.52 (m, | |
| | 2H), 2.18-2.11 (m, 2H). | |
| Example 188 | 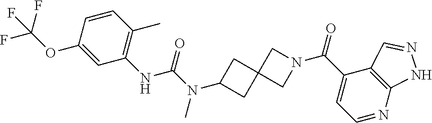 [SMILES: Cc1ccc(OC(F)(F)F)cc1NC(=O)N(C)C1CC2(C1)CN(C(=O)c1ccnc3[nH]ncc13)C2] | 39 mg (48%) |
| | LCMS (ESI): Method 2, t_{R} = 4.12 min, m/z (M + 1) = 489.7 ¹H NMR (400 MHz, DMSO-d6) δ | |
| | 13.88 (s, 1H), 8.62 (d, J = 4.3 Hz, 1H), 8.25 (d, J = 1.1 Hz, 1H), 7.80 (d, J = 7.79 Hz, 1H), 7.33 | |
| | (t, J = 5.4, Hz, 2H), 7.27 (dd, J = 4.4, 8.4 Hz, 1H), 7.02-6.98 (m, 1H), 4.58-4.44 (m, 1H), 4.37 | |
| | (s, 1H), 4.23 (s, 2H), 4.11 (s, 1H), 2.87 (d, J = 13.8 Hz, 3H), 2.44-2.38 (m, 4H), 2.17 (d, J = | |
| | 5.1 Hz, 3H). | |
| Example 189 | 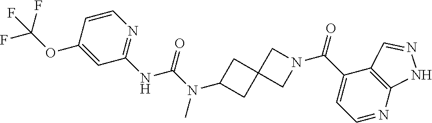 [SMILES: CN(C(=O)Nc1cc(OC(F)(F)F)ccn1)C1CC2(C1)CN(C(=O)c1ccnc3[nH]ncc13)C2] | 41 mg (51%) |
| | LCMS (ESI): Method 2, t_{R} = 3.64 min, m/z (M + 1) = 476.6 ¹H NMR (400 MHz, DMSO-d6) δ | |
| | 13.87 (s, 1H), 9.29 (d, J = 16.5 Hz, 1H), 8.62 (t, J = 5.2 Hz, 1H), 8.33 (dd, J = 5.8, 7.5 Hz, 1H), | |
| | 8.25 (d, J = 2.9 Hz, 1H), 7.82 (d, J = 1.8 Hz, 1H), 7.33 (dd, J = 4.7, 9.6 Hz, 1H), 6.98 (s, 1H), | |
| | 4.63-4.45 (m, 1H), 4.36 (s, 1H), 4.23 (s, 2H), 4.10 (s, 1H), 2.87 (d, J = 13.0 Hz, 3H), 2.47- | |
| | 2.36 (m, 4H). | |
| Example 190 | 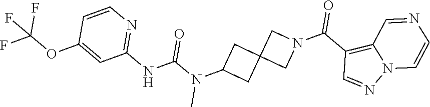 [SMILES: CN(C(=O)Nc1cc(OC(F)(F)F)ccn1)C1CC2(C1)CN(C(=O)c1cnn3ccncc13)C2] | 34 mg (41%) |
| | LCMS (ESI): Method 2, t_{R} = 3.77 min, m/z (M + 1) = 476.5 ¹H NMR (400 MHz, DMSO-d6) δ | |
| | 9.56 (d, J = 1.5 Hz, 1H), 9.34 (s, 1H), 8.92 (dd, J = 1.1, 4.6 Hz, 1H), 8.44 (d, J = 7.8 Hz, 1H), | |
| | 8.35 (d, J = 5.6 Hz, 1H), 8.12 (d, J = 4.6 Hz, 1H), 7.84 (s, 1H), 7.00-6.98 (m, 1H), 4.63-4.57 | |
| | (m, 2H), 4.48 (s, 1H), 4.19 (s, 1H), 4.05 (s, 1H), 2.90 (s, 3H), 2.47-2.37 (m, 4H). | |
| Example 193 | 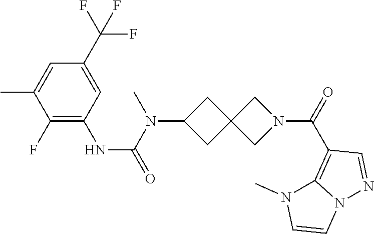 [SMILES: Cc1cc(C(F)(F)F)cc(NC(=O)N(C)C2CC3(C2)CN(C(=O)c2cnn4ccn(C)c24)C3)c1F] | 13 mg (18%) |
| | LCMS (ESI): Method 5, t_{R} = 5.58 min, m/z (M + 1) = 493.27 ¹H NMR (400 MHz, DMSO-d6) δ | |
| | ppm 8.21 (1 H, s), 7.77 (1 H, s), 7.73 (1 H, m), 7.66 (1 H, d, J = 2.19 Hz), 7.40 (1 H, br d, J = | |
| | 4.60 Hz), 7.26 (1 H, s), 4.52 (1 H, br t, J = 8.44 Hz), 4.22 (4 H, m), 3.96 (3 H, s), 2.88 (3 H, s), | |
| | 2.39 (4 H, m), 2.30 (3 H, d, J = 1.32 Hz) | |
| Example 197 | 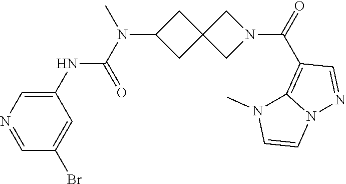 [SMILES: CN(C(=O)Nc1cncc(Br)c1)C1CC2(C1)CN(C(=O)c1cnn3ccn(C)c13)C2] | 48 mg (56%) |
| | LCMS (ESI): Method 2, t_{R} = 3.25 min, m/z (M + 1) = 472.1 ¹H NMR (400 MHz, DMSO-d6) δ | |
| | 8.67-8.64 (m, 2H), 8.25 (d, J = 2.1 Hz, 1H), 8.19 (dd, J = 2.1, 2.1 Hz, 1H), 7.77 (s, 1H), 7.66 | |
| | (d, J = 2.3 Hz, 1H), 7.26-7.25 (m, 1H), 4.59-4.49 (m, 1H), 4.43-4.02 (m, 4H), 3.96 (s, 3H), 2.89 | |
| | (s, 3H), 2.43-2.36 (m, 4H). | |
| Example 198 | 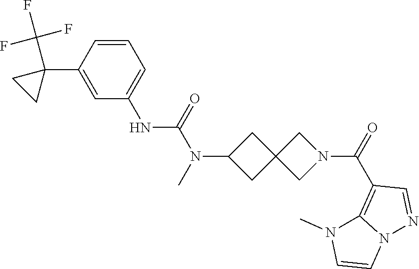 [SMILES: CN(C(=O)Nc1cccc(C2(C(F)(F)F)CC2)c1)C1CC2(C1)CN(C(=O)c1cnn3ccn(C)c13)C2] | 43 mg (47%) |
| | LCMS (ESI): Method 2, t_{R} = 4.33 min, m/z (M + 1) = 501.3 ¹H NMR (400 MHz, DMSO-d6) δ | |
| | 8.33 (s, 1H), 7.77 (s, 1H), 7.66 (d, J = 2.1 Hz, 1H), 7.59 (s, 1H), 7.47 (d, J = 8.5 Hz, 1H), 7.26- | |
| | 7.20 (m, 2H), 7.02 (d, J = 7.6 Hz, 1H), 4.61-4.51 (m, 1H), 4.45-4.02 (m, 4H), 3.96 (s, 3H), 2.87 | |
| | (s, 3H), 2.42-2.31 (m, 4H), 1.33-1.29 (m, 2H), 1.07 (s, 2H). | |
| Example 199 | 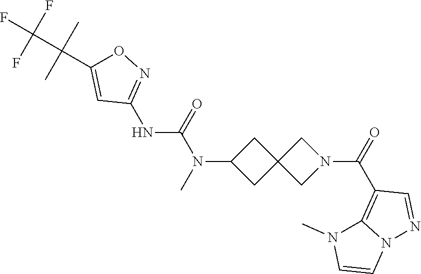 [SMILES: CN(C(=O)Nc1cc(C(C)(C)C(F)(F)F)on1)C1CC2(C1)CN(C(=O)c1cnn3ccn(C)c13)C2] | 43 mg (47%) |
| | LCMS (ESI): Method 2, t_{R} = 3.97 min, m/z (M + 1) = 494.2 ¹H NMR (400 MHz, DMSO-d6) δ | |
| | 9.64 (s, 1H), 7.76 (s, 1H), 7.66 (d, J = 2.3 Hz, 1H), 7.25 (s, 1H), 6.80 (s, 1H), 4.55-4.44 (m, | |
| | 1H), 4.40-4.01 (m, 4H), 3.96 (s, 3H), 2.85 (s, 3H), 2.40-2.31 (m, 4H), 1.52 (s, 6H). | |
| Example 200 | 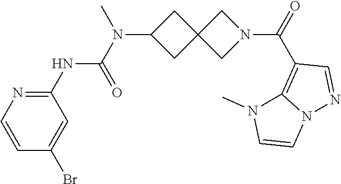 [SMILES: CN(C(=O)Nc1cc(Br)ccn1)C1CC2(C1)CN(C(=O)c1cnn3ccn(C)c13)C2] | 41 mg (47%) |
| | LCMS (ESI): Method 2, t_{R} = 3.38 min, m/z (M + 1) = 472.2 ¹H NMR (400 MHz, DMSO-d6) δ | |
| | 9.15 (s, 1H), 8.14 (d, J = 5.3 Hz, 1H), 8.05 (d, J = 1.8 Hz, 1H), 7.76 (s, 1H), 7.66 (d, J = 2.1 Hz, | |
| | 1H), 7.27-7.25 (m, 1H), 7.23 (dd, J = 1.9, 5.4 Hz, 1H), 4.60-4.50 (m, 1H), 4.44-4.02 (m, 4H), | |
| | 3.96 (s, 3H), 2.88 (s, 3H), 2.45-2.32 (m, 4H). | |
| Example 204 | 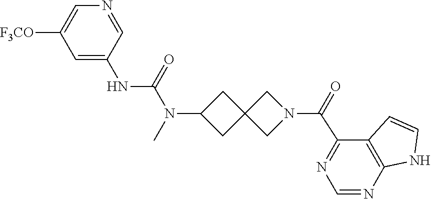 [SMILES: CN(C(=O)Nc1cncc(OC(F)(F)F)c1)C1CC2(C1)CN(C(=O)c1ncnc3[nH]ccc13)C2] | 38 mg (43%) |
| | LCMS (ESI): Method 2, t_{R} = 3.61 min, m/z (M + 1) = 476.4 ¹H NMR (400 MHz, DMSO-d6) δ | |
| | 12.34 (s, 1H), 8.81 (d, J = 5.8 Hz, 1H), 8.84-8.75 (m, 1H), 8.69 (t, J = 2.5 Hz, 1H), 8.22 (d, J = | |
| | 2.1 Hz, 1H), 8.07 (s, 1H), 7.69 (d, J = 3.4 Hz, 1H), 6.91 (d, J = 3.4 Hz, 1H), 4.78-4.48 (m, 3H), | |
| | 4.28-4.06 (m, 2H), 2.94-2.87 (m, 3H), 2.47-2.35 (m, 4H). | |
| Example 205 | 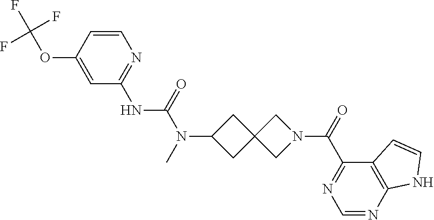 [SMILES: CN(C(=O)Nc1cc(OC(F)(F)F)ccn1)C1CC2(C1)CN(C(=O)c1ncnc3[nH]ccc13)C2] | 50 mg (56%) |
| | LCMS (ESI): Method 2, t_{R} = 3.79 min, m/z (M + 1) = 476.4 ¹H NMR (400 MHz, DMSO-d6) δ | |
| | 12.32 (s, 1H), 9.30 (s, 1H), 8.81 (d, J = 6.1 Hz, 1H), 8.34 (dd, J = 2.0, 5.7 Hz, 1H), 7.84 (s, 1H), | |
| | 7.69 (d, J = 3.4 Hz, 1H), 7.01-6.95 (m, 1H), 6.90 (d, J = 3.4 Hz, 1H), 4.76-4.47 (m, 3H), 4.26- | |
| | 4.04 (m, 2H), 2.91-2.84 (m, 3H), 2.49-2.30 (m, 4H). | |
| Example 206 | 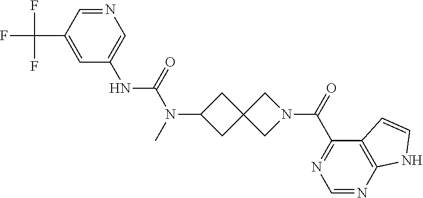 [SMILES: CN(C(=O)Nc1cncc(C(F)(F)F)c1)C1CC2(C1)CN(C(=O)c1ncnc3[nH]ccc13)C2] | 41 mg (47%) |
| | LCMS (ESI): Method 2, t_{R} = 3.49 min, m/z (M + 1) = 460.3 ¹H NMR (400 MHz, DMSO-d6) δ | |
| | 12.33 (s, 1H), 8.94 (t, J = 2.6 Hz, 1H), 8.87-8.78 (m, 1H), 8.81 (d, J = 5.8 Hz, 1H), 8.52 (s, 1H), | |
| | 8.33-8.28 (m, 1H), 7.69 (d, J = 3.4 Hz, 1H), 6.91 (d, J = 3.4 Hz, 1H), 4.78-4.44 (m, 3H), 4.27- | |
| | 4.03 (m, 2H), 2.96-2.86 (m, 3H), 2.50-2.35 (m, 4H). | |
| Example 207 | 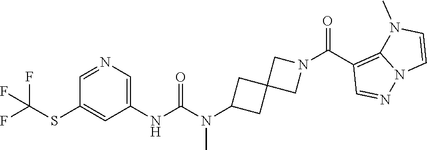 [SMILES: CN(C(=O)Nc1cncc(SC(F)(F)F)c1)C1CC2(C1)CN(C(=O)c1cnn3ccn(C)c13)C2] | 45 mg (41%) |
| | LCMS (ESI): Method 2, t_{R} = 3.67 min, m/z (M + 1) = 464.5 ¹H NMR (400 MHz, DMSO-d6) δ | |
| | 8.85 (d, J = 2.3 Hz, 1H), 8.78 (s, 1H), 8.39 (d, J = 1.9 Hz, 1H), 8.33 (s, 1H), 7.77 (s, 1H), 7.66 | |
| | (d, J = 2.2 Hz, 1H), 7.26 (s, 1H), 4.64-4.49 (m, 1H), 4.49-3.78 (m, 4H), 3.96 (s, 3H), 2.91 (s, | |
| | 3H), 2.47-2.32 (m, 4H) | |
| Example 213 | 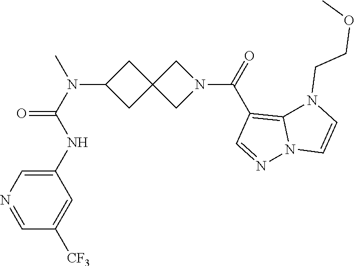 [SMILES: COCCn1ccn2ncc(C(=O)N3CC4(CC(N(C)C(=O)Nc5cncc(C(F)(F)F)c5)C4)C3)c12] | 7 mg (9%) |
| | LCMS (ESI): Method 5, t_{R} = 5.13 min, m/z (M + 1) = 506.50 ¹H NMR (400 MHz, DMSO-d6) δ | |
| | ppm 8.94 (1 H, d, J = 2.19 Hz), 8.84 (1 H, s), 8.52 (1 H, s), 8.30 (1 H, s), 7.78 (1 H, s), 7.65 (1 | |
| | H, d, J = 2.19 Hz), 7.27 (1 H, s), 4.60 (3 H, m), 4.11 (4 H, m), 3.60 (2 H, t, J = 5.15 Hz), 3.21 (3 | |
| | H, s), 2.92 (3 H, s), 2.41 (4 H, m). | |
| Example 215 | 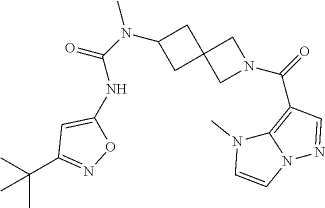 [SMILES: CN(C(=O)Nc1cc(C(C)(C)C)no1)C1CC2(C1)CN(C(=O)c1cnn3ccn(C)c13)C2] | 15 mg (3%) |
| | LCMS (ESI): Method 5, t_{R} = 4.97 min, m/z (M + 1) = 440.50 ¹H NMR (400 MHz, DMSO-d6) δ | |
| | ppm 10.02 (1 H, s), 7.76 (1 H, s), 7.66 (1 H, d, J = 2.19 Hz), 7.26 (1 H, dd, J = 2.08, 1.21 Hz), | |
| | 5.99 (1 H, s), 4.47 (1 H, br t, J = 8.55 Hz), 3.96 (3 H, s), 4.22 (4 H, m), 2.85 (3 H, s), 2.37 (4 H, | |
| | m), 1.23 (9 H, s). | |
| Example 217 | 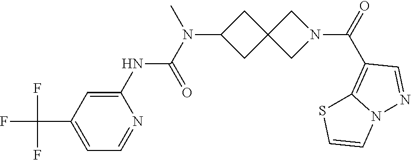 [SMILES: CN(C(=O)Nc1cc(C(F)(F)F)ccn1)C1CC2(C1)CN(C(=O)c1cnn3ccsc13)C2] | 54 mg (53%) |
| | LCMS (ESI): Method 11, t_{R} = 3.90 min, m/z (M + 1) = 465.2 ¹H NMR (400 MHz, DMSO-d6) δ | |
| | 9.43 (s, 1H), 8.50 (d, J = 5.3 Hz, 1H), 8.32 (d, J = 4.1 Hz, 1H), 8.14 (s, 1H), 8.07 (s, 1H), 7.53 | |
| | (dd, J = 1.4, 4.1 Hz, 1H), 7.32-7.30 (m, 1H), 4.62-4.38 (m, 3H), 4.11-3.96 (m, 2H), 2.91 (s, 3H), | |
| | 2.48-2.35 (m, 4H). | |
| Example 219 | 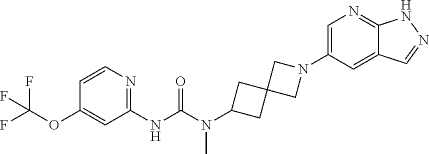 [SMILES: CN(C(=O)Nc1cc(OC(F)(F)F)ccn1)C1CC2(C1)CN(c1cnc3[nH]ncc3c1)C2] | 46mg (37%) |
| | LCMS (ESI): Method 5, t_{R} = 5.59 min, m/z (M + 1) = 448.40 ¹H NMR (600 MHz, DMSO-d6) δ | |
| | ppm 13.29 (1 H, br s), 9.35 (1 H, s), 8.35 (1 H, d, J = 5.58 Hz), 7.95 (1 H, d, J = 2.35 Hz), 7.92 (1 | |
| | H, s), 7.85 (1 H, s), 7.10 (1 H, d, J = 2.35 Hz), 6.99 (1 H, dt, J = 4.62, 1.06 Hz), 4.61 (1 H, m), | |
| | 3.95 (2 H, s), 3.82 (2 H, s), 2.91 (3 H, s), 2.40 (4 H, m). | |
| Example 221 | 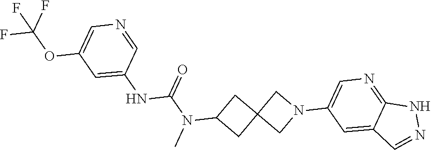 [SMILES: CN(C(=O)Nc1cncc(OC(F)(F)F)c1)C1CC2(C1)CN(c1cnc3[nH]ncc3c1)C2] | 12 mg (36%) |
| | LCMS (ESI): Method 5, t_{R} = 5.16 min, m/z (M + 1) = 448.40 ¹H NMR (600 MHz, DMSO-d6) δ | |
| | ppm 13.28 (1 H, br s), 8.83 (1 H, s), 8.70 (1 H, d, J = 2.05 Hz), 8.22 (1 H, d, J = 2.35 Hz), 8.08 | |
| | (1 H, s), 7.94 (1 H, d, J = 2.49 Hz), 7.91 (1 H, s), 7.10 (1 H, d, J = 2.20 Hz), 4.60 (1 H, t, J = | |
| | 8.51 Hz), 3.95 (2 H, s), 3.83 (2 H, s), 2.93 (3 H, s), 2.42 (4 H, d, J = 8.66 Hz). | |
| Example 222 | 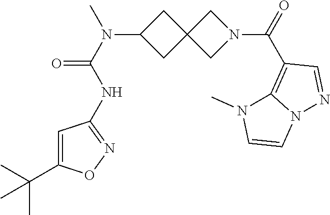 [SMILES: CN(C(=O)Nc1cc(C(C)(C)C)on1)C1CC2(C1)CN(C(=O)c1cnn3ccn(C)c13)C2] | 45 mg (56%) |
| | LCMS (ESI): Method 5, t_{R} = 5.18 min, m/z (M + 1) = 440.50 ¹H NMR (400 MHz, DMSO-d6) δ | |
| | ppm 9.44 (1 H, s), 7.76 (1 H, s), 7.66 (1 H, d, J = 2.19 Hz), 7.26 (1 H, dd, J = 2.08, 1.21 Hz), 6.43 | |
| | (1 H, s), 4.50 (1 H, s), 3.96 (3 H, s), 4.20 (4 H, m), 2.84 (3 H, s), 2.36 (4 H, m), 1.27 (9 H, s). | |
| Example 223 | 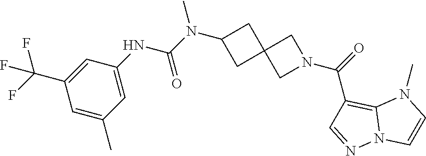 [SMILES: Cc1cc(NC(=O)N(C)C2CC3(C2)CN(C(=O)c2cnn4ccn(C)c24)C3)cc(C(F)(F)F)c1] | 15 mg (14%) |
| | LCMS (ESI): Method 7, t_{R} = 0.88 min, m/z (M + 1) = 475 ¹H NMR (400 MHz, DMSO-d6) δ | |
| | ppm 8.51 (1 H, s), 7.77 (1 H, s), 7.72 (1 H, s), 7.66 (1 H, d, J = 2.19 Hz), 7.57 (1 H, s), 7.26 (1 | |
| | H, dd, J = 2.08, 1.21 Hz), 7.09 (1 H, s), 4.53 (1 H, t, J = 8.55 Hz), 3.96 (3 H, s), 4.26 (4 H, m), | |
| | 2.88 (3 H, s), 2.39 (4 H, m), 2.33 (3 H, s). | |
| Example 224 | 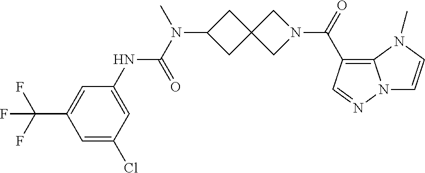 [SMILES: CN(C(=O)Nc1cc(Cl)cc(C(F)(F)F)c1)C1CC2(C1)CN(C(=O)c1cnn3ccn(C)c13)C2] | 90 mg (64%) |
| | LCMS (ESI): Method 5, t_{R} = 6.54 min, m/z (M + 1) = 495.40 ¹H NMR (400 MHz, DMSO-d6) δ | |
| | ppm 8.77 (1 H, s), 7.93 (1 H, s), 7.88 (1 H, s), 7.77 (1 H, m), 7.66 (1 H, d, J = 2.19 Hz), 7.36 (1 | |
| | H, s), 7.26 (1 H, dd, J = 2.08, 1.21 Hz), 4.53 (1 H, t, J = 8.55 Hz), 3.96 (3 H, s), 4.23 (4 H, m), | |
| | 2.89 (3 H, s), 2.40 (4 H, m). | |
| Example 226 | 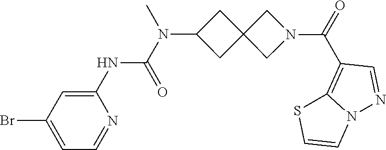 [SMILES: CN(C(=O)Nc1cc(Br)ccn1)C1CC2(C1)CN(C(=O)c1cnn3ccsc13)C2] | 50 mg (48%) |
| | LCMS (ESI): Method 11, t_{R} = 3.70 min, m/z (M+) = 475.2 ¹H NMR (400 MHz, DMSO-d6) δ | |
| | 9.16 (s, 1H), 8.32 (d, J = 4.1 Hz, 1H), 8.14 (d, J = 5.4 Hz, 1H), 8.08-8.05 (m, 2H), 7.53 (dd, J = | |
| | 1.4, 4.1 Hz, 1H), 7.23 (dd, J = 1.8, 5.4 Hz, 1H), 4.61-4.37 (m, 3H), 4.15-3.94 (m, 2H), 2.89 (s, | |
| | 3H), 2.47-2.32 (m, 4H). | |
| Example 228 | 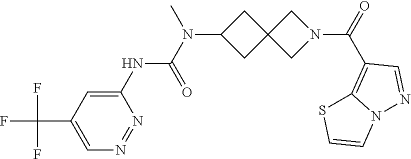 [SMILES: CN(C(=O)Nc1cc(C(F)(F)F)cnn1)C1CC2(C1)CN(C(=O)c1cnn3ccsc13)C2] | 58 mg (57%) |
| | LCMS (ESI): Method 11, t_{R} = 3.52 min, m/z (M + 1) = 466.2 ¹H NMR (400 MHz, DMSO-d6) δ | |
| | 10.22 (s, 1H), 9.26 (s, 1H), 8.34-8.31 (m, 2H), 8.07 (s, 1H), 7.53 (dd, J = 1.4, 4.1 Hz, 1H), 4.66- | |
| | 4.41 (m, 3H), 4.14-3.97 (m, 2H), 2.95 (s, 3H), 2.46-2.39 (m, 4H). | |
| Example 232 | 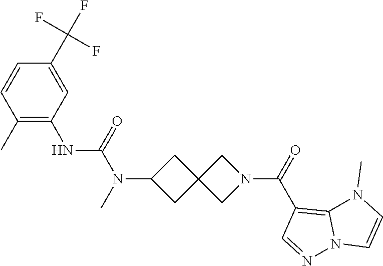 [SMILES: Cc1ccc(C(F)(F)F)cc1NC(=O)N(C)C1CC2(C1)CN(C(=O)c1cnn3ccn(C)c13)C2] | 28 mg (39%) |
| | LCMS (ESI): Method 12, t_{R} = 4.03 min, m/z (M + 1) = 475.2 ¹H NMR (400 MHz, DMSO-d6) δ | |
| | 7.93 (s, 1H), 7.77 (s, 1H), 7.66 (d, J = 2.3 Hz, 1H), 7.63 (s, 1H), 7.42-7.34 (m, 2H), 7.26-7.26 | |
| | (m, 1H), 4.59-4.50 (m, 1H), 4.19-4.19 (m, 4H), 3.96 (s, 3H), 2.90 (s, 3H), 2.42-2.38 (m, 4H), | |
| | 2.25 (s, 3H). | |
| Example 235 | 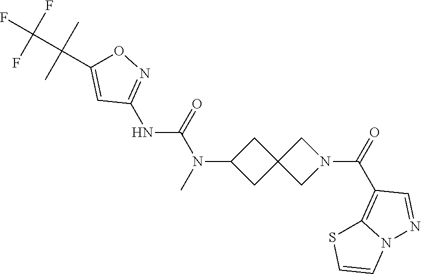 [SMILES: CN(C(=O)Nc1cc(C(C)(C)C(F)(F)F)on1)C1CC2(C1)CN(C(=O)c1cnn3ccsc13)C2] | 59 mg (55%) |
| | LCMS (ESI): Method 12, t_{R} = 4.06 min, m/z (M + 1) = 497.4 ¹H NMR (400 MHz, DMSO-d6) δ | |
| | 9.65 (s, 1H), 8.32 (d, J = 4.1 Hz, 1H), 8.05 (s, 1H), 7.53 (dd, J = 1.4, 4.1 Hz, 1H), 6.81 (s, 1H), | |
| | 4.56-4.36 (m, 3H), 4.15-3.93 (m, 2H), 2.86 (s, 3H), 2.41-2.35 (m, 4H), 1.53 (s, 6H). | |
| Example 254 | 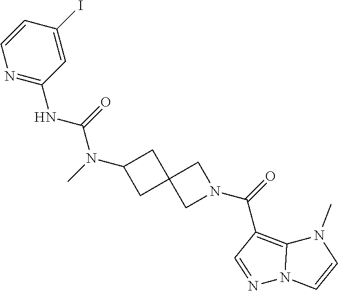 [SMILES: CN(C(=O)Nc1cc(I)ccn1)C1CC2(C1)CN(C(=O)c1cnn3ccn(C)c13)C2] | 43 mg (33%) |
| | LCMS (ESI): Method 11, t_{R} = 3.50 min, m/z (M + 1) = 520.3 ¹H NMR (400 MHz, DMSO-d6) δ | |
| | 9.02 (s, 1H), 8.25 (d, J = 1.0 Hz, 1H), 7.96 (d, J = 4.9 Hz, 1H), 7.76 (s, 1H), 7.66 (d, J = 2.3 Hz, | |
| | 1H), 7.37 (dd, J = 1.5, 5.3 Hz, 1H), 7.26-7.26 (m, 1H), 4.59-4.49 (m, 1H), 4.18-4.18 (m, 4H), | |
| | 3.96 (s, 3H), 2.87 (s, 3H), 2.44-2.31 (m, 4H). | |
| Example 255 | 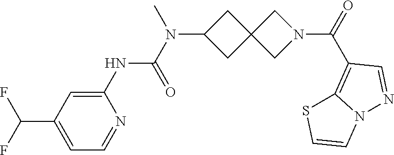 [SMILES: CN(C(=O)Nc1cc(C(F)F)ccn1)C1CC2(C1)CN(C(=O)c1cnn3ccsc13)C2] | 25 mg (25%) |
| | LCMS (ESI): Method 12, t_{R} = 3.44 min, m/z (M + 1) = 447.5 ¹H NMR (400 MHz, DMSO-d6) δ | |
| | 9.19 (s, 1H), 8.39 (d, J = 5.0 Hz, 1H), 8.32 (d, J = 4.1 Hz, 1H), 8.07 (s, 1H), 8.00 (s, 1H), 7.53 | |
| | (dd, J = 1.4, 4.1 Hz, 1H), 7.13 (d, J = 5.0 Hz, 1H), 7.06 (t, J = 55.4 Hz, 1H), 4.63-4.35 (m, 3H), | |
| | 4.17-3.94 (m, 2H), 2.90 (s, 3H), 2.48-2.34 (m, 4H). | |
| Example 256 | 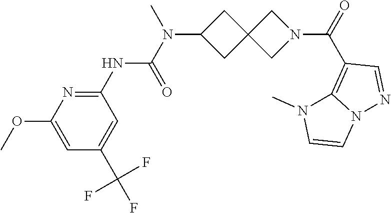 [SMILES: COc1cc(C(F)(F)F)cc(NC(=O)N(C)C2CC3(C2)CN(C(=O)c2cnn4ccn(C)c24)C3)n1] | 25 mg (25%) |
| | LCMS (ESI): Method 11, t_{R} = 4.41 min, m/z (M + 1) = 492.5 ¹H NMR (400 MHz, DMSO-d6) δ | |
| | 9.06 (s, 1H), 7.77 (s, 1H), 7.67-7.66 (m, 2H), 7.26-7.26 (m, 1H), 6.70 (s, 1H), 4.60-4.49 (m, 1H), | |
| | 4.51-4.00 (m, 4H), 3.96 (s, 3H), 3.91 (s, 3H), 2.90 (s, 3H), 2.46-2.33 (m, 4H). | |
| Example 257 | 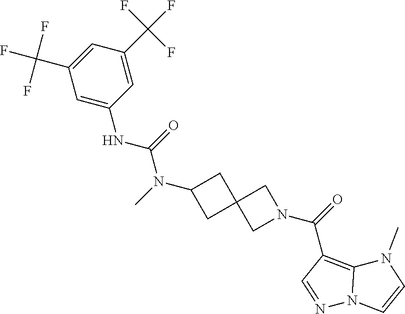 [SMILES: CN(C(=O)Nc1cc(C(F)(F)F)cc(C(F)(F)F)c1)C1CC2(C1)CN(C(=O)c1cnn3ccn(C)c13)C2] | 89 mg (66%) |
| | LCMS (ESI): Method 12, t_{R} = 4.69 min, m/z (M + 1) = 529.2 ¹H NMR (400 MHz, DMSO-d6) δ | |
| | 8.96 (s, 1H), 8.23 (s, 2H), 7.77 (s, 1H), 7.66 (d, J = 2.3 Hz, 1H), 7.59 (s, 1H), 7.26-7.26 (m, 1H), | |
| | 4.61-4.51 (m, 1H), 4.19-4.19 (m, 4H), 3.96 (s, 3H), 2.91 (s, 3H), 2.43-2.38 (m, 4H). | |
| Example 262 | 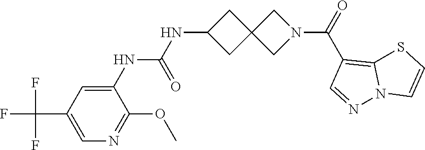 [SMILES: COc1ncc(C(F)(F)F)cc1NC(=O)NC1CC2(C1)CN(C(=O)c1cnn3ccsc13)C2] | 7 mg (6%) |
| | LCMS (ESI): Method 11, t_{R} = 4.07 min, m/z (M + 1) = 481.3 ¹H NMR (400 MHz, DMSO-d6) δ | |
| | 8.66 (d, J = 2.3 Hz, 1H), 8.32 (d, J = 4.1 Hz, 2H), 8.08 (d, J = 8.0 Hz, 2H), 7.53 (dd, J = 1.4, 4.1 | |
| | Hz, 1H), 7.34 (d, J = 7.2 Hz, 1H), 4.59-4.37 (m, 2H), 4.03 (s, 6H), 2.62-2.55 (m, 3H), 2.13-2.05 | |
| | (m, 2H). | |
| Example 263 | 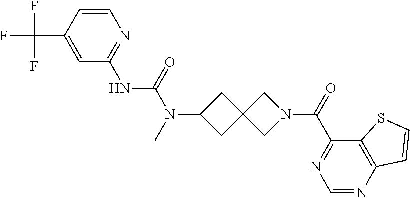 [SMILES: CN(C(=O)Nc1cc(C(F)(F)F)ccn1)C1CC2(C1)CN(C(=O)c1ncnc3ccsc13)C2] | 50 mg (50%) |
| | LCMS (ESI): Method 11, t_{R} = 4.41 min, m/z (M + 1) = 477.6 ¹H NMR (400 MHz, DMSO-d6) δ | |
| | 9.43 (s, 1H), 9.29 (d, J = 6.3 Hz, 1H), 8.60 (d, J = 5.6 Hz, 1H), 8.51 (d, J = 5.0 Hz, 1H), 8.16 | |
| | (s, 1H), 7.69 (dd, J = 2.0, 5.6 Hz, 1H), 7.32 (dd, J = 0.7, 4.2 Hz, 1H), 4.91 (s, 1H), 4.77 (s, 1H), | |
| | 4.60 (q, J = 8.4 Hz, 1H), 4.28 (s, 1H), 4.15 (s, 1H), 2.91 (s, 3H), 2.49-2.39 (m, 4H). | |
| Example 264 | 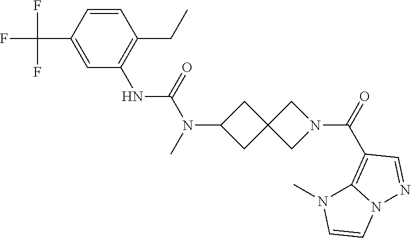 [SMILES: CCc1ccc(C(F)(F)F)cc1NC(=O)N(C)C1CC2(C1)CN(C(=O)c1cnn3ccn(C)c13)C2] | 75 mg (60%) |
| | LCMS (ESI): Method 11, t_{R} = 4.19 min, m/z (M + 1) = 489.6 ¹H NMR (400 MHz, DMSO-d6) δ | |
| | 7.94 (s, 1H), 7.77 (d, J = 1.1 Hz, 1H), 7.66 (d, J = 2.2 Hz, 1H), 7.57 (s, 1H), 7.44-7.42 (m, 2H), | |
| | 7.26 (dd, J = 1.1, 2.2 Hz, 1H), 4.60-4.48 (m, 1H), 4.48-3.79 (m, 4H), 3.96 (s, 3H), 2.90 (s, 3H), | |
| | 2.63 (q, J = 7.5 Hz, 2H), 2.46-2.33 (m, 4H), 1.14 (t, J = 7.5 Hz, 3H). | |
| Example 265 | 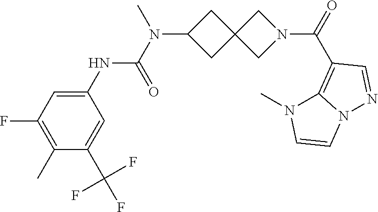 [SMILES: Cc1c(F)cc(NC(=O)N(C)C2CC3(C2)CN(C(=O)c2cnn4ccn(C)c24)C3)cc1C(F)(F)F] | 67 mg (53%) |
| | LCMS (ESI): Method 11, t_{R} = 4.42 min, m/z (M + 1) = 493.6 ¹H NMR (400 MHz, DMSO-d6) δ | |
| | 8.68 (s, 1H), 7.68 (d, J = 1.2 Hz, 1H), 7.75-7.70 (m, 1H), 7.71 (s, 1H), 7.66 (d, J = 2.2 Hz, 1H), | |
| | 7.26 (dd, J = 1.2, 2.2 Hz, 1H), 4.60-4.47 (m, 1H), 4.47-3.80 (m, 4H), 3.96 (s, 3H), 2.88 (s, 3H), | |
| | 2.45-2.31 (m, 4H), 2.26-2.21 (m, 3H). | |
| Example 266 | 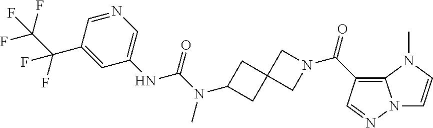 [SMILES: CN(C(=O)Nc1cncc(C(F)(F)C(F)(F)F)c1)C1CC2(C1)CN(C(=O)c1cnn3ccn(C)c13)C2] | 42 mg (32%) |
| | LCMS (ESI): Method 11, t_{R} = 3.18 min, m/z (M + 1) = 512.5 ¹H NMR (400 MHz, DMSO-d6) δ | |
| | 8.98 (d, J = 2.3 Hz, 1H), 8.87 (s, 1H), 8.46 (d, J = 1.9 Hz, 1H), 8.30 (br s, 1H), 7.77 (d, J = 1.0 | |
| | Hz, 1H), 7.66 (d, J = 2.2 Hz, 1H), 7.26 (dd, J = 1.2, 2.2 Hz, 1H), 4.63-4.51 (m, 1H), 4.51-3.87 | |
| | (m, 4H), 3.96 (s, 3H), 2.92 (s, 3H), 2.46-2.34 (m, 4H). | |
| Example 268 | 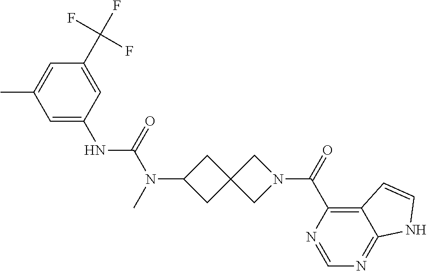 [SMILES: Cc1cc(NC(=O)N(C)C2CC3(C2)CN(C(=O)c2ncnc4[nH]ccc24)C3)cc(C(F)(F)F)c1] | 73 mg (74%) |
| | LCMS (ESI): Method 11, t_{R} = 4.26 min, m/z (M + 1) = 473.6 ¹H NMR (400 MHz, DMSO-d6) δ | |
| | 12.33 (bs, 1H), 8.81 (d, J = 6.3 Hz, 1H), 8.50 (d, J = 9.0 Hz, 1H), 7.72-7.68 (m, 2H), 7.57 (s, 1H), | |
| | 7.09 (s, 1H), 6.91 (d, J = 3.1 Hz, 1H), 4.73 (s, 1H), 4.59 (s, 1H), 4.57-4.45 (m, 1H), 4.22 (s, 1H), | |
| | 4.09 (s, 1H), 2.87 (d, J = 3.9 Hz, 3H), 2.46-2.35 (m, 4H), 2.33 (s, 3H). | |
| Example 269 | 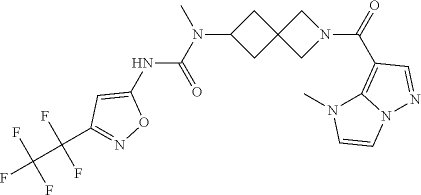 [SMILES: CN(C(=O)Nc1cc(C(F)(F)C(F)(F)F)no1)C1CC2(C1)CN(C(=O)c1cnn3ccn(C)c13)C2] | 17 mg (16%) |
| | LCMS (ESI): Method 11, t_{R} = 3.87 min, m/z (M + 1) = 494.5 ¹H NMR (400 MHz, DMSO-d6) δ | |
| | 10.27 (s, 1H), 7.76 (s, 1H), 7.66 (d, J = 2.3 Hz, 1H), 7.26-7.26 (m, 1H), 6.13 (s, 1H), 4.53-4.43 | |
| | (m, 1H), 4.38-4.05 (m, 4H), 3.96 (s, 3H), 2.86 (s, 3H), 2.42-2.33 (m, 4H), 1.48 (s, 6H). | |
| Example 270 | 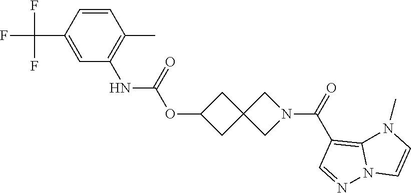 [SMILES: Cc1ccc(C(F)(F)F)cc1NC(=O)OC1CC2(C1)CN(C(=O)c1cnn3ccn(C)c13)C2] | 25 mg (18%) |
| | LCMS (ESI): Method 11, t_{R} = 4.35 min, m/z (M + 1) = 462.7 ¹H NMR (400 MHz, DMSO-d6) δ | |
| | 9.15 (s, 1H), 7.79 (s, 1H), 7.76 (s, 1H), 7.66 (d, J = 2.3 Hz, 1H), 7.44-7.36 (m, 2H), 7.27-7.25 | |
| | (m, 1H), 4.92-4.86 (m, 1H), 4.51-4.02 (m, 4H), 3.96 (s, 3H), 2.71-2.63 (m, 2H), 2.35-2.30 (m, | |
| | 2H), 2.29 (s, 3H). | |
| Example 271 | 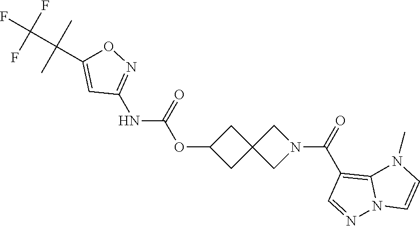 [SMILES: Cn1ccn2ncc(C(=O)N3CC4(CC(OC(=O)Nc5cc(C(C)(C)C(F)(F)F)on5)C4)C3)c12] | 28 mg (19%) |
| | LCMS (ESI): Method 11, t_{R} = 4.09 min, m/z (M + 1) = 481.6 ¹H NMR (400 MHz, DMSO-d6) | |
| | δ 10.79 (s, 1H), 7.76 (s, 1H), 7.67 (d, J = 2.1 Hz, 1H), 7.26-7.26 (m, 1H), 6.78 (s, 1H), 4.88 | |
| | (quint, J = 7.1 Hz, 1H), 4.77-4.06 (m, 4H), 3.96 (s, 3H), 2.70-2.63 (m, 2H), 2.34-2.26 (m, | |
| | 2H), 1.54 (s, 6H). | |
| Example 280 | 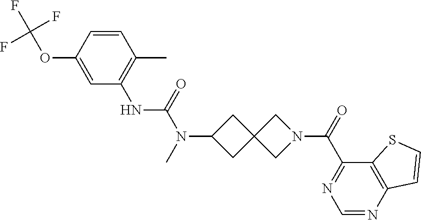 [SMILES: Cc1ccc(OC(F)(F)F)cc1NC(=O)N(C)C1CC2(C1)CN(C(=O)c1ncnc3ccsc13)C2] | 30 mg (28%) |
| | LCMS (ESI): Method 11, t_{R} = 4.78 min, m/z (M + 1) = 506.4 ¹H NMR (400 MHz, DMSO-d6) δ | |
| | 9.28 (d, J = 1.9 Hz, 1H), 8.60 (d, J = 5.5 Hz, 1H), 7.82 (d, J = 3.9 Hz, 1H), 7.68 (d, J = 5.3 Hz, | |
| | 1H), 7.35 (s, 1H), 7.28 (d, J = 8.5 Hz, 1H), 7.01 (dd, J = 1.6, 8.3 Hz, 1H), 4.89 (s, 1H), 4.77 (s, | |
| | 1H), 4.61-4.50 (m, 1H), 4.28 (s, 1H), 4.15 (s, 1H), 2.90 (s, 3H), 2.47-2.40 (m, 4H), 2.19 (s, 3H). | |
| Example 281 | 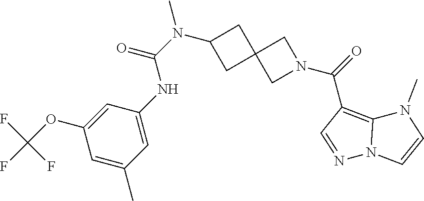 [SMILES: Cc1cc(NC(=O)N(C)C2CC3(C2)CN(C(=O)c2cnn4ccn(C)c24)C3)cc(OC(F)(F)F)c1] | 42 mg (39%) |
| | LCMS (ESI): Method 2, t_{R} = 4.35 min, m/z (M + 1) = 491.5 ¹H NMR (400 MHz, DMSO-d6) δ | |
| | 8.45 (s, 1H), 7.77 (s, 1H), 7.66 (d, J = 2.1 Hz, 1H), 7.43 (s, 1H), 7.27 (d, J = 9.4 Hz, 2H), 6.73 | |
| | (s, 1H), 4.58-4.48 (m, 1H), 4.22-4.21 (m, 4H), 3.96 (s, 3H), 2.87 (s, 3H), 2.40-2.31 (m, 4H), | |
| | 2.28 (s, 3H). | |
| Example 282 | 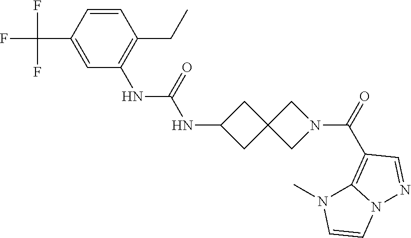 [SMILES: CCc1ccc(C(F)(F)F)cc1NC(=O)NC1CC2(C1)CN(C(=O)c1cnn3ccn(C)c13)C2] | 49 mg (38%) |
| | LCMS (ESI): Method 11, t_{R} = 4.96 min, m/z (M + 1) = 475.5 ¹H NMR (400 MHz, DMSO-d6) δ | |
| | 8.31 (s, 1H), 7.80 (s, 1H), 7.77 (d, J = 1.0 Hz, 1H), 7.66 (d, J+2.2 Hz, 1H), 7.35 (d, J = 7.9 Hz, | |
| | 1H), 7.26 (dd, J = 1.0, 2.2 Hz, 1H), 7.23 (dd, J = 1.2, 7.9 Hz, 1H), 7.04 (d, J = 7.3 Hz, 1H), 4.63- | |
| | 3.69 (m, 5H), 3.96 (s, 3H), 2.66-2.50 (m, 4H), 2.15-2.04 (m, 2H), 1.16 (t, J = 7.5 Hz, 3H). | |
| Example 283 | 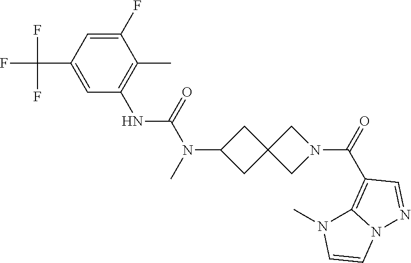 [SMILES: Cc1c(F)cc(C(F)(F)F)cc1NC(=O)N(C)C1CC2(C1)CN(C(=O)c1cnn3ccn(C)c13)C2] | 49 mg (38%) |
| | LCMS (ESI): Method 18, t_{R} = 6.31 min, m/z (M + 1) = 493.5 ¹H NMR (400 MHz, DMSO-d6) δ | |
| | 8.19 (s, 1H), 7.77 (d, J = 1.1 Hz, 1H), 7.66 (d, J = 2.2 Hz, 1H), 7.50 (s, 1H), 7.37 (d, J = 9.8 Hz, | |
| | 1H), 7.26 (dd, J = 1.1, 2.2 Hz, 1H), 4.61-4.48 (m, 1H), 4.48-3.80 (m, 4H), 3.96 (s, 3H), 2.91 (s, | |
| | 3H), 2.46-2.35 (m, 4H), 2.11 (s, 3H). | |
| Example 284 | 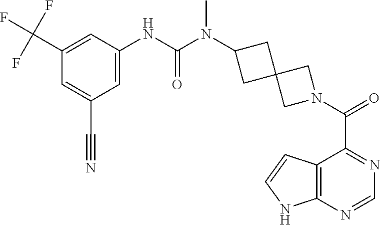 [SMILES: CN(C(=O)Nc1cc(C#N)cc(C(F)(F)F)c1)C1CC2(C1)CN(C(=O)c1ncnc3[nH]ccc13)C2] | 66 mg (55%) |
| | LCMS (ESI): Method 11, t_{R} = 4.05 min, m/z (M + 1) = 484.5 ¹H NMR (400 MHz, DMSO-d6) δ | |
| | 12.33 (bs, 1H), 8.98-8.85 (bs, 1H), 8.81 (d, J = 6.4 Hz, 1H), 8.23-8.18 (m, 2H), 7.85 (s, 1H), | |
| | 7.69 (d, J = 3.1 Hz, 1H), 6.91 (d, J = 3.1 Hz, 1H), 4.74 (s, 1H), 4.59 (s, 1H), 4.58-4.49 (m, 1H), | |
| | 4.23 (s, 1H), 4.09 (s, 1H), 2.90 (d, J = 3.9 Hz, 3H), 2.47-2.39 (m, 4H). | |
| Example 285 | 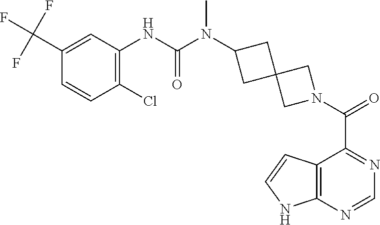 [SMILES: CN(C(=O)Nc1cc(C(F)(F)F)ccc1Cl)C1CC2(C1)CN(C(=O)c1ncnc3[nH]ccc13)C2] | 87 mg (73%) |
| | LCMS (ESI): Method 11, t_{R} = 4.52 min, m/z (M + 1) = 493.5 ¹H NMR (400 MHz, DMSO-d6) δ | |
| | 12.33 (bs, 1H), 8.81 (d, J = 6.4 Hz, 1H), 8.75 (d, J = 10.6 Hz, 1H), 7.93 (s, 1H), 7.87 (s, 1H), | |
| | 7.69 (d, J = 3.4 Hz, 1H), 7.36 (s, 1H), 6.91 (d, J = 3.4 Hz, 1H), 4.73 (s, 1H), 4.59 (s, 1H), 4.57- | |
| | 4.45 (m, 1H), 4.22 (s, 1H), 4.09 (s, 1H), 2.89 (d, J = 3.9 Hz, 3H), 2.47-2.38 (m, 4H). | |
| Example 286 | 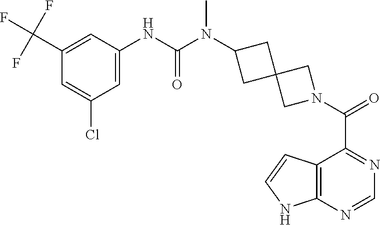 [SMILES: CN(C(=O)Nc1cc(Cl)cc(C(F)(F)F)c1)C1CC2(C1)CN(C(=O)c1ncnc3[nH]ccc13)C2] | 9 mg (8%) |
| | LCMS (ESI): Method 11, t_{R} = 4.35 min, m/z (M + 1) = 493.5 ¹H NMR (400 MHz, DMSO-d6) | |
| | δ 12.30 (bs, 1H), 8.80 (d, J = 2.8 Hz, 1H), 8.04-8.01 (m, 2H), 7.72-7.68 (m, 2H), 7.48-7.43 (m, | |
| | 1H), 6.91 (d, J = 3.5 Hz, 1H), 4.72 (s, 1H), 4.59 (s, 1H), 4.57-4.48 (m, 1H), 4.22 (s, 1H), 4.09 | |
| | (s, 1H), 2.91 (d, J = 2.9 Hz, 3H), 2.47-2.39 (m, 4H). | |
| Example 287 | 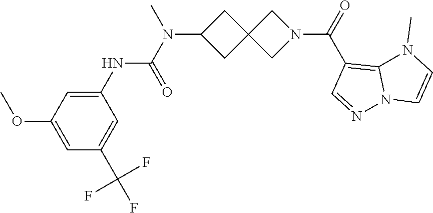 [SMILES: COc1cc(NC(=O)N(C)C2CC3(C2)CN(C(=O)c2cnn4ccn(C)c24)C3)cc(C(F)(F)F)c1] | 38 mg (42%) |
| | LCMS (ESI): Method 11, t_{R} = 4.12 min, m/z (M + 1) = 491.5 ¹H NMR (400 MHz, DMSO-d6) | |
| | δ 8.57 (s, 1H), 7.77 (s, 1H), 7.67 (d, J = 2.1 Hz, 1H), 7.51 (s, 1H), 7.44 (dd, J = 2.2, 2.2 Hz, | |
| | 1H), 7.27-7.26 (m, 1H), 6.80 (s, 1H), 4.60-4.50 (m, 1H), 4.48-4.03 (m, 4H), 3.97 (s, 3H), | |
| | 3.79 (s, 3H), 2.89 (s, 3H), 2.46-2.34 (m, 4H). | |
| Example 288 | 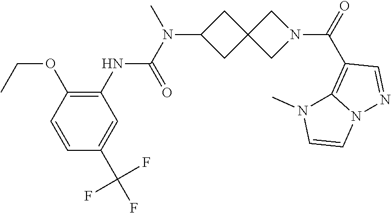 [SMILES: CCOc1ccc(C(F)(F)F)cc1NC(=O)N(C)C1CC2(C1)CN(C(=O)c1cnn3ccn(C)c13)C2] | 41 mg (32%) |
| | LCMS (ESI): Method 11, t_{R} = 4.49 min, m/z (M + 1) = 505.5 ¹H NMR (400 MHz, DMSO-d6) δ | |
| | 8.20 (d, J = 2.2 Hz, 1H), 7.77 (s, 1H), 7.66 (d, J = 2.2 Hz, 1H), 7.48 (s, 1H), 7.33-7.29 (m, 1H), | |
| | 7.27-7.25 (m, 1H), 7.17 (d, J = 8.6 Hz, 1H), 4.53-3.85 (m, 5H), 4.18 (q, J = 6.9 Hz, 2H), 3.96 | |
| | (s, 3H), 2.89 (s, 3H), 2.50-2.29 (m, 4H, partly covered by residual DMSO peak), 1.42 (t, J = 6.9 | |
| | Hz, 3H). | |
| Example 289 | 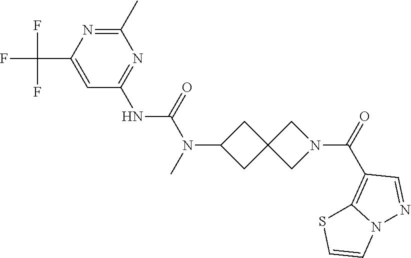 [SMILES: Cc1nc(NC(=O)N(C)C2CC3(C2)CN(C(=O)c2cnn4ccsc24)C3)cc(C(F)(F)F)n1] | 25 mg (51%) |
| | LCMS (ESI): Method 11, t_{R} = 3.78 min, m/z (M + 1) = 480.3 ¹H NMR (400 MHz, DMSO-d6) δ | |
| | 10.11 (br s, 1H), 8.32 (d, J = 4.1 Hz, 1H), 8.07 (br s, 1H), 7.98 (s, 1H), 7.53 (dd, J = 1.4, 4.1 Hz, | |
| | 1H), 4.63-4.28 (m, 3H), 4.20-3.85 (m, 2H), 2.90 (s, 3H), 2.57 (s, 3H), 2.47-2.35 (m, 4H). | |
| Example 290 | 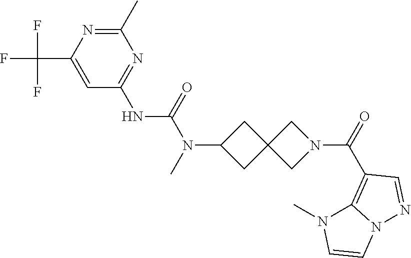 [SMILES: Cc1nc(NC(=O)N(C)C2CC3(C2)CN(C(=O)c2cnn4ccn(C)c24)C3)cc(C(F)(F)F)n1] | 58 mg (47%) |
| | LCMS (ESI): Method 11, t_{R} = 3.64 min, m/z (M + 1) = 477.4 ¹H NMR (400 MHz, DMSO-d6) | |
| | δ 10.11 (br s, 1H), 7.98 (s, 1H), 7.76 (d, J = 1.2 Hz, 1H), 7.66 (d, J = 2.2 Hz, 1H), 7.26 (dd, J = | |
| | 1.2, 2.2 Hz, 1H), 4.61-4.49 (m, 1H), 4.49-3.81 (m, 4H), 3.96 (s, 3H), 2.90 (s, 3H), 2.57 (s, 3H), | |
| | 2.46-2.30 (m, 4H). | |
| Example 291 | 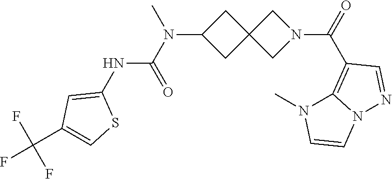 [SMILES: CN(C(=O)Nc1cc(C(F)(F)F)cs1)C1CC2(C1)CN(C(=O)c1cnn3ccn(C)c13)C2] | 42 mg (35%) |
| | LCMS (ESI): Method 11, t_{R} = 4.05 min, m/z (M + 1) = 467.6 ¹H NMR (400 MHz, DMSO-d6) δ | |
| | 9.81 (br s, 1H), 7.77 (d, J = 1.0 Hz, 1H), 7.66 (d, J = 2.2 Hz, 1H), 7.45 (t, J = 1.5 Hz, 1H), 7.26 | |
| | (dd, J = 1.0, 2.2 Hz, 1H), 6.78 (d, J = 1.8 Hz, 1H), 4.62-4.50 (m, 1H), 4.50-3.80 (m, 4H), 3.96 | |
| | (s, 3H), 2.88 (s, 3H), 2.43-2.35 (m, 4H). | |
| Example 293 | 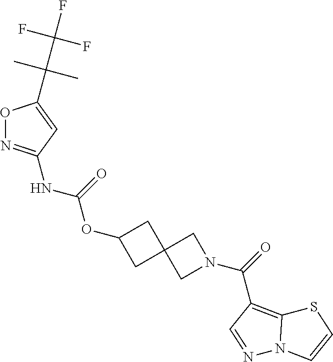 [SMILES: CC(C)(c1cc(NC(=O)OC2CC3(C2)CN(C(=O)c2cnn4ccsc24)C3)no1)C(F)(F)F] | 40 mg (22%) |
| | LCMS (ESI): Method 11, t_{R} = 4.25 min, m/z (M + 1) = 484.3 ¹H NMR (400 MHz, DMSO-d6) δ | |
| | 10.79 (s, 1H), 8.31 (d, J = 4.1 Hz, 1H), 8.05 (d, J = 1.3 Hz, 1H), 7.53 (dd, J = 1.4, 4.1 Hz, 1H), | |
| | 6.78 (s, 1H), 4.92-4.84 (m, 1H), 4.54-4.41 (m, 2H), 4.12-3.98 (m, 2H), 2.72-2.65 (m, 2H), 2.35- | |
| | 2.28 (m, 2H), 1.54 (s, 6H). | |
| Example 298 | 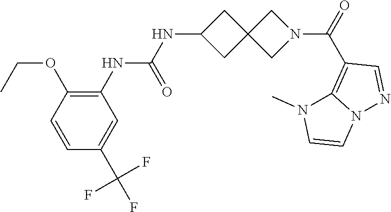 [SMILES: CCOc1ccc(C(F)(F)F)cc1NC(=O)NC1CC2(C1)CN(C(=O)c1cnn3ccn(C)c13)C2] | 15 mg (11%) |
| | LCMS (ESI): Method 17, t_{R} = 6.85 min, m/z (M + 1) = 491.6 ¹H NMR (400 MHz, DMSO-d6) δ | |
| | 8.50 (d, J = 2.2 Hz, 1H), 7.92 (s, 1H), 7.78 (d, J = 1.2 Hz, 1H), 7.67 (d, J = 2.3 Hz, 1H), 7.41 (d, | |
| | J = 7.2 Hz, 1H), 7.26 (dd, J = 1.2, 2.2 Hz, 1H), 7.21 (ddd, J = 0.7, 2.3, 8.5 Hz, 1H), 7.12 (d, J = | |
| | 8.5 Hz, 1H), 4.50-3.90 (m, 5H), 4.19 (q, J = 7.0 Hz, 2H), 3.97 (s, 3H), 2.60-2.53 (m, 2H), 2.13- | |
| | 2.02 (m, 2H), 1.42 (t, J = 7.0 Hz, 3H). | |
| Example 300 | 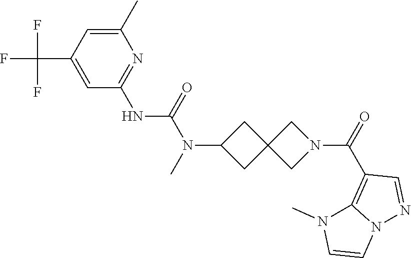 [SMILES: Cc1cc(C(F)(F)F)cc(NC(=O)N(C)C2CC3(C2)CN(C(=O)c2cnn4ccn(C)c24)C3)n1] | 38 mg (31%) |
| | LCMS (ESI): Method 11, t_{R} = 4.18 min, m/z (M + 1) = 476.4 ¹H NMR (400 MHz, DMSO-d6) δ | |
| | 9.32 (s, 1H), 7.95 (s, 1H), 7.77 (d, J = 0.9 Hz, 1H), 7.66 (d, J = 2.2 Hz, 1H), 7.26 (dd, J = 0.9, | |
| | 2.2 Hz, 1H), 7.19 (s, 1H), 4.63-4.51 (m, 1H), 4.51-3.86 (m, 4H), 3.96 (s, 3H), 2.89 (s, 3H), 2.49 | |
| | (s, 3H), 2.46-2.29 (m, 4H). | |
| Example 303 | 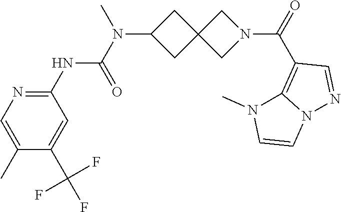 [SMILES: Cc1cnc(NC(=O)N(C)C2CC3(C2)CN(C(=O)c2cnn4ccn(C)c24)C3)cc1C(F)(F)F] | 60 mg (49%) |
| | LCMS (ESI): Method 11, t_{R} = 3.96 min, m/z (M + 1) = 476.5 ¹H NMR (400 MHz, DMSO-d6) δ | |
| | 9.27 (s, 1H), 8.34 (s, 1H), 8.12 (s, 1H), 7.76 (d, J = 0.9 Hz, 1H), 7.66 (d, J = 2.2 Hz, 1H), 7.26 | |
| | (dd, J = 0.9, 2.2 Hz, 1H), 4.63-4.51 (m, 1H), 4.51-3.81 (m, 4H), 3.96 (s, 3H), 2.89 (s, 3H), 2.45- | |
| | 2.30 (m, 7H). | |
| Example 304 | 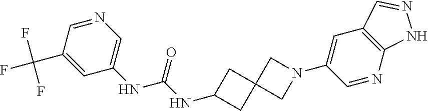 [SMILES: O=C(Nc1cncc(C(F)(F)F)c1)NC1CC2(C1)CN(c1cnc3[nH]ncc3c1)C2] | 13 mg (10%) |
| | LCMS (ESI): Method 11, t_{R} = 3.36 min, m/z (M + 1) = 418.4 ¹H NMR (400 MHz, DMSO-d6) δ | |
| | 13.27 (s, 1H), 9.04-9.02 (m, 1H), 8.72 (d, J = 2.1 Hz, 1H), 8.47 (s, 1H), 8.37 (s, 1H), 7.94-7.90 | |
| | (m, 2H), 7.09 (d, J = 2.5 Hz, 1H), 6.88 (d, J = 7.5 Hz, 1H), 4.13-4.05 (m, 1H), 3.92 (s, 2H), 3.82 | |
| | (s, 2H), 2.22-2.14 (m, 2H). | |
| Example 305 | 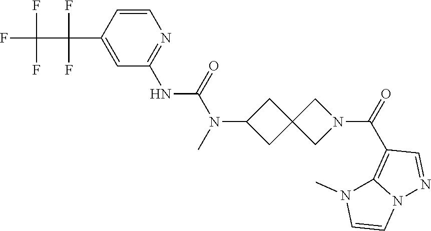 [SMILES: CN(C(=O)Nc1cc(C(F)(F)C(F)(F)F)ccn1)C1CC2(C1)CN(C(=O)c1cnn3ccn(C)c13)C2] | 37 mg (28%) |
| | LCMS (ESI): Method 11, t_{R} = 4.12 min, m/z (M + 1) = 512.6 ¹H NMR (400 MHz, DMSO-d6) δ | |
| | 9.44 (s, 1H), 8.52 (d, J = 5.2 Hz, 1H), 8.16 (s, 1H), 7.76 (d, J = 1.0 Hz, 1H), 7.66 (d, J = 2.2 Hz, | |
| | 1H), 7.28 (dd, J = 1.2, 5.2 Hz, 1H), 7.26 (dd, J = 1.0, 2.2 Hz, 1H), 4.63-4.52 (m, 1H), 4.53-3.81 | |
| | (m, 4H), 3.96 (s, 3H), 2.90 (s, 3H), 2.46-2.31 (m, 4H). | |
| Example 306 | 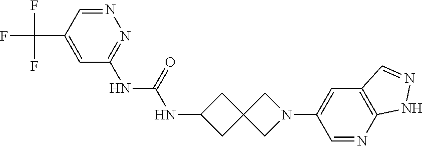 [SMILES: O=C(Nc1cc(C(F)(F)F)cnn1)NC1CC2(C1)CN(c1cnc3[nH]ncc3c1)C2] | 23 mg (18%) |
| | LCMS (ESI): Method 11, t_{R} = 3.33 min, m/z (M + 1) = 419.5 ¹H NMR (400 MHz, DMSO-d6) δ | |
| | 13.20 (s, 1H), 9.28-9.27 (m, 1H), 8.10 (d, J = 5.5 Hz, 1H), 7.94-7.90 (m, 3H), 7.77 (d, J = 1.5 | |
| | Hz, 1H), 7.18 (dd, J = 1.8, 5.5 Hz, 1H), 7.08 (d, J = 2.6 Hz, 1H), 4.11 (dd, J = 7.9, 15.7 Hz, 1H), | |
| | 3.92 (s, 2H), 3.82 (s, 2H), 2.61-2.54 (m, 2H), 2.19-2.07 (m, 2H). | |
| Example 307 | 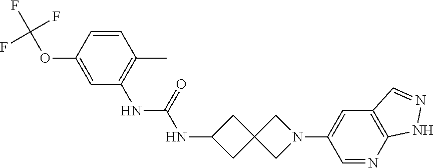 [SMILES: Cc1ccc(OC(F)(F)F)cc1NC(=O)NC1CC2(C1)CN(c1cnc3[nH]ncc3c1)C2] | 35 mg (25%) |
| | LCMS (ESI): Method 11, t_{R} = 4.24 min, m/z (M + 1) = 447.5 ¹H NMR (400 MHz, DMSO-d6) δ | |
| | 13.27 (s, 1H), 8.03-8.02 (m, 1H), 7.94-7.90 (m, 2H), 7.76 (s, 1H), 7.22 (d, J = 8.3 Hz, 1H), 7.10- | |
| | 7.05 (m, 2H), 6.82 (dd, J = 1.6, 8.3 Hz, 1H), 4.07 (dd, J = 7.8, 15.5 Hz, 1H), 3.92 (s, 2H), 3.83 | |
| | (s, 2H), 2.60-2.53 (m, 2H), 2.19 (s, 3H), 2.14-2.08 (m, 2H). | |
| Example 309 | 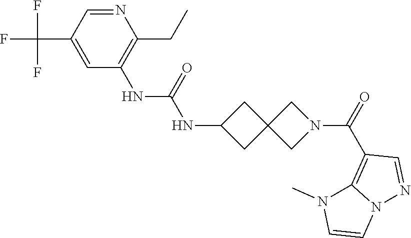 [SMILES: CCc1ncc(C(F)(F)F)cc1NC(=O)NC1CC2(C1)CN(C(=O)c1cnn3ccn(C)c13)C2] | 66 mg (51%) |
| | LCMS (ESI): Method 11, t_{R} = 3.61 min, m/z (M + 1) = 476.6 ¹H NMR (400 MHz, DMSO-d6) δ | |
| | 8.64 (d, J = 1.9 Hz, 1H), 8.46-8.44 (m, 1H), 8.06 (s, 1H), 7.77 (d, J = 1.1 Hz, 1H), 7.66 (d, J = | |
| | 2.2 Hz, 1H), 7.26 (dd, J = 1.1, 2.2 Hz, 1H), 7.14 (d, J = 7.3 Hz, 1H), 4.53-3.84 (m, 5H), 3.96 | |
| | (s, 3H), 2.80 (q, J = 7.5 Hz, 2H), 2.60-2.53 (m, 2H), 2.17-2.05 (m, 2H), 1.23 (t, J = 7.5 Hz, 3H). | |
| Example 310 | 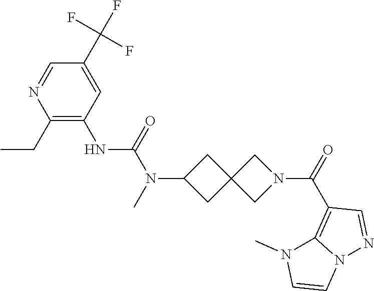 [SMILES: CCc1ncc(C(F)(F)F)cc1NC(=O)N(C)C1CC2(C1)CN(C(=O)c1cnn3ccn(C)c13)C2] | 40 mg (32%) |
| | LCMS (ESI): Method 11, t_{R} = 3.60 min, m/z (M + 1) = 490.6 ¹H NMR (400 MHz, DMSO-d6) δ | |
| | 8.64 (s, 1H), 8.12 (s, 1H), 7.80 (d, J = 1.7 Hz, 1H), 7.76 (s, 1H), 7.66 (d, J = 2.1 Hz, 1H), 7.26 | |
| | (s, 1H), 4.61-4.49 (m, 1H), 4.49-3.89 (m, 4H), 3.96 (s, 3H), 2.92 (s, 3H), 2.81 (q, J = 7.4 Hz, | |
| | 2H), 2.46-2.35 (m, 4H), 1.20 (t, J = 7.4 Hz, 3H). | |
| Example 312 | 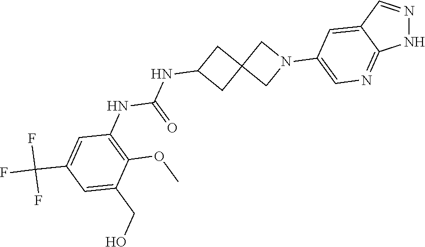 [SMILES: COc1c(CO)cc(C(F)(F)F)cc1NC(=O)NC1CC2(C1)CN(c1cnc3[nH]ncc3c1)C2] | 17 mg (16%) |
| | LCMS (ESI): Method 11, t_{R} = 3.50 min, m/z (M + 1) = 477.4 ¹H NMR (400 MHz, DMSO-d6) δ | |
| | 13.28 (s, 1H), 8.47 (d, J = 2.1 Hz, 1H), 8.21 (s, 1H), 7.95-7.90 (m, 2H), 7.31 (d, J = 7.3 Hz, 2H), | |
| | 7.09 (d, J = 2.4 Hz, 1H), 5.34 (dd, J = 5.7, 5.7 Hz, 1H), 4.59 (d, J = 5.6 Hz, 2H), 4.06 (dd, J = | |
| | 7.8, 15.3 Hz, 1H), 3.92 (s, 2H), 3.83 (s, 2H), 3.72 (s, 3H), 2.61-2.55 (m, 2H), 2.14-2.07 (m, 2H). | |
| Intermediate 98 | 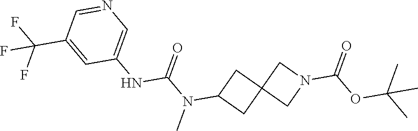 [SMILES: CN(C(=O)Nc1cncc(C(F)(F)F)c1)C1CC2(C1)CN(C(=O)OC(C)(C)C)C2] | 90 mg (27%) |
| | LCMS (ESI): Method 1, t_{R} = 1.68 min, m/z (M − 1) = 413.3 | |
| Intermediate 99 | 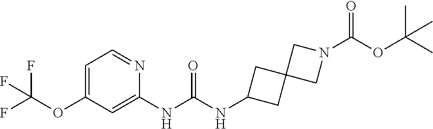 [SMILES: CC(C)(C)OC(=O)N1CC2(CC(NC(=O)Nc3cc(OC(F)(F)F)ccn3)C2)C1] | 150 mg (quantitative) |
| | LCMS (ESI): Method 4, t_{R} = 1.75 min, m/z (M + 1 − tBu) = 361.0 | |
| Intermediate 174 | 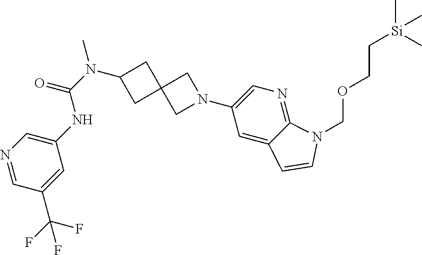 [SMILES: CN(C(=O)Nc1cncc(C(F)(F)F)c1)C1CC2(C1)CN(c1cnc3c(ccn3COCC[Si](C)(C)C)c1)C2] | 103 mg (27%) |

LCMS (ESI): Method 13, t_{R} = 2.53 min, m/z (M − 1) = 561.19

### Example 292: 2-(pyrazolo[5,1-b]thiazole-7-carbonyl)-2-azaspiro[3.3]heptan-6-yl (6-(trifluoromethyl)pyrimidin-4-yl)carbamate

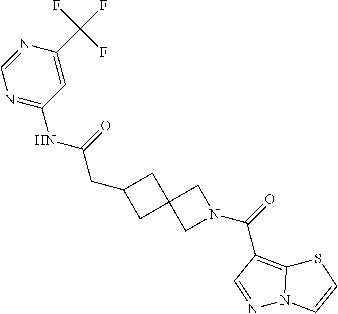 [SMILES: O=C(CC1CC2(C1)CN(C(=O)c1cnn3ccsc13)C2)Nc1cc(C(F)(F)F)ncn1]

To a stirred solution of (6-hydroxy-2-azaspiro[3.3]heptan-2-yl) (pyrazolo[5,1-b]thiazol-7-yl) methanone (100 mg, 0.380 mmol) in DMF (1 mL) sodium hydride (45 mg, 1.1 mmol) was added and the mixture was stirred for 20 minutes, then 2,2,2-trichloroethyl (6-(trifluoromethyl)pyrimidin-4-yl)carbamate (231 mg, 0.684 mmol) was added and the mixture was stirred overnight.

The crude was purified by preparative HPLC (Xbridge C18 19×150 mm, 10 μm 20-80% ACN/H₂O/H₂O (10 mM NH₄CO₃), 20 mL/min, r.t.) to give the title compound (48 mg, 0.105 mmol, 28% yield)

LCMS (ESI): Method 11, t_{R}=3.84 min, m/z (M+1)=453.4

¹H NMR (400 MHz, DMSO-d6) δ 11.37 (bs, 1H), 9.02 (s, 1H), 8.31 (d, J=4.1 Hz, 1H), 8.20-8.18 (m, 1H), 8.07-8.05 (m, 1H), 7.54-7.51 (m, 1H), 4.99-4.92 (m, 1H), 4.49-4.49 (m, 2H), 4.07-4.07 (m, 2H), 2.76-2.68 (m, 2H), 2.39-2.31 (m, 2H).

The following Examples were prepared analogously to what described for Example 292 (Table 41).

TABLE 41

| | | Product Amount |
|---|---|---|
| | Structure | (Yield) |
| Nº | Analytical Data | |
| Example 294 | 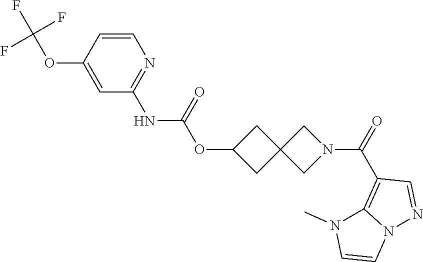 [SMILES: Cn1ccn2ncc(C(=O)N3CC4(CC(OC(=O)Nc5cc(OC(F)(F)F)ccn5)C4)C3)c12] | 70 mg (39%) |
| | LCMS (ESI): Method 11, t_{R} = 4.04 min, m/z (M + 1) = 465.5 | |
| | ¹H NMR (400 MHZ, DMSO-d6) δ 10.64-10.59 (m, 1H), 8.37 (d, J = 5.8 | |
| | Hz, 1H), 7.79-7.79 (m, 1H), 7.76 (d, J = 1.0 Hz, 1H), 7.67 (d, J-2.3 Hz, | |
| | 1H), 7.26-7.26 (m, 1H), 7.05 (d, J-5.1 Hz, 1H), 4.95-4.86 (m, 1H), 4.50- | |
| | 4.05 (m, 4H), 3.97 (s, 3H), 2.72-2.65 (m, 2H), 2.34-2.27 (m, 2H). | |
| Example 295 | 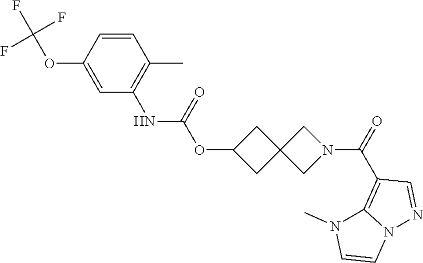 [SMILES: Cc1ccc(OC(F)(F)F)cc1NC(=O)OC1CC2(C1)CN(C(=O)c1cnn3ccn(C)c13)C2] | 70 mg (38%) |
| | LCMS (ESI): Method 11, t_{R} = 4.50 min, m/z (M + 1) = 478.3 | |
| | ¹H NMR (400 MHZ, CDCl₃) δ 9.07 (s, 1H), 7.76 (s, 1H), 7.67 (d, J = 2.1 | |
| | Hz, 1H), 7.49 (s, 1H), 7.29 (d, J = 8.4 Hz, 1H), 7.26 (dd, J = 1.2, 2.1 Hz, | |
| | 1H), 7.02 (dd, J = 1.5, 8.3 Hz, 1H), 4.93-4.84 (m, 1H), 4.47-4.04 (m, | |
| | 4H), 3.97 (s, 3H), 2.71-2.64 (m, 2H), 2.35-2.27 (m, 2H), 2.22 (s, 3H). | |
| Example 296 | 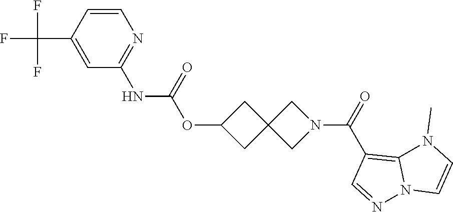 [SMILES: Cn1ccn2ncc(C(=O)N3CC4(CC(OC(=O)Nc5cc(C(F)(F)F)ccn5)C4)C3)c12] | 9 mg (10%) |
| | LCMS (ESI): Method 11, t_{R} = 3.99 min, m/z (M + 1) = 449.5 | |
| | ¹H NMR (400 MHZ, DMSO-d6) δ 10.72 (s, 1H), 8.54 (d, J = 5.1 Hz, 1H), | |
| | 8.11 (s, 1H), 7.77-7.75 (m, 1H), 7.66 (d, J-2.3 Hz, 1H), 7.42-7.39 (m, | |
| | 1H), 7.27-7.24 (m, 1H), 4.95-4.87 (m, 1H), 4.20-4.20 (m, 4H), 3.96 | |
| | (s, 3H), 2.72-2.65 (m, 2H), 2.35-2.28 (m, 2H). | |
| Example 297 | 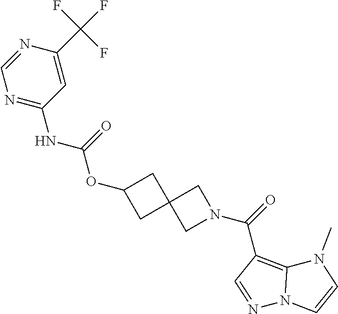 [SMILES: Cn1ccn2ncc(C(=O)N3CC4(CC(OC(=O)Nc5cc(C(F)(F)F)ncn5)C4)C3)c12] | 15 mg (9%) |
| | LCMS (ESI): Method 11, t_{R} = 3.75 min, m/z (M + 1) = 450.6 | |
| | ¹H NMR (400 MHZ, DMSO-d6) δ 11.35 (s, 1H), 9.00 (s, 1H), 8.18 (s, | |
| | 1H), 7.77-7.75 (m, 1H), 7.66 (d, J = 2.3 Hz, 1H), 7.27-7.25 (m, 1H), | |
| | 4.98-4.89 (m, 1H), 4.60-4.01 (m, 4H), 3.96 (s, 3H), 2.73-2.66 (m, 2H), | |
| | 2.36-2.29 (m, 2H). | |
| Example 299 | 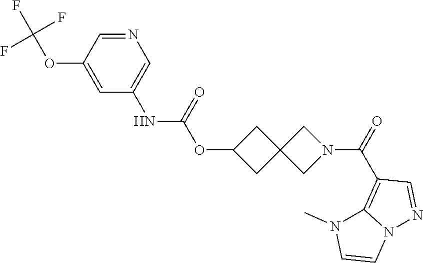 [SMILES: Cn1ccn2ncc(C(=O)N3CC4(CC(OC(=O)Nc5cncc(OC(F)(F)F)c5)C4)C3)c12] | 92 mg (52%) |
| | LCMS (ESI): Method 11, t_{R} = 3.87 min, m/z (M + 1) = 465.3 | |
| | ¹H NMR (400 MHZ, DMSO-d6) δ 10.33-10.29 (m, 1H), 8.55 (s, 1H), | |
| | 8.24 (s, 1H), 8.02 (s, 1H), 7.76 (s, 1H), 7.67 (d, J = 2.3 Hz, 1H), 7.26-7.26 | |
| | (m, 1H), 4.95-4.86 (m, 1H), 4.47-4.05 (m, 4H), 3.97 (s, 3H), 2.72-2.64 | |
| | (m, 2H), 2.34-2.26 (m, 2H). | |
| Example 311 | 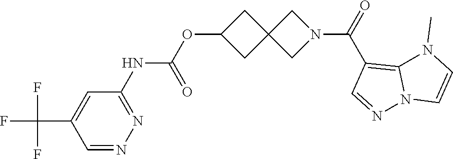 [SMILES: Cn1ccn2ncc(C(=O)N3CC4(CC(OC(=O)Nc5cc(C(F)(F)F)cnn5)C4)C3)c12] | 58 mg (21%) |

LCMS (ESI): Method 11, t_{R} = 3.51 min, m/z (M + 1) = 450.5
¹H NMR (400 MHZ, DMSO-d6) δ 11.41 (s, 1H), 9.37-9.35 (m, 1H), 8.36
(s, 1H), 7.76 (s, 1H), 7.66 (d, J = 2.3 Hz, 1H), 7.27-7.25 (m, 1H), 4.99-
4.90 (m, 1H), 4.62-4.01 (m, 4H), 3.96 (s, 3H), 2.74-2.66 (m, 2H), 2.38-2.30 (m, 2H).

### Example 202: 1-(2-(1-(2-methoxyethyl)-1H-imidazo[1,2-b]pyrazole-7-carbonyl)-2-azaspiro[3.3]heptan-6-yl)-1-methyl-3-(4-(trifluoromethoxy)pyridin-2-yl)urea

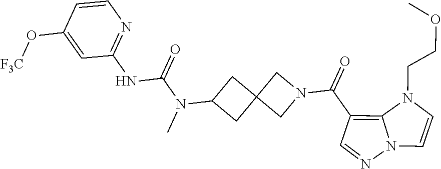 [SMILES: COCCn1ccn2ncc(C(=O)N3CC4(CC(N(C)C(=O)Nc5cc(OC(F)(F)F)ccn5)C4)C3)c12]

Intermediate 155 was dissolved in DMF (1 mL) then DIPEA (0.235 mL, 1.34 mmol) was added, followed by addition of phenyl (4-(trifluoromethoxy)pyridin-2-yl)carbamate (66.8 mg, 0.22 mmol) at 0° C. The reaction was stirred at rt for 12 h. The reaction was diluted with EtOAc and then washed with brine. The organic layer was concentrated under vacuum.

The crude was purified by RF FCC to give the title compound (20 mg, 0.04 mmol, 17% yield).

LCMS (ESI): Method 5, t_{R}=5.88 min, m/z (M+1)=522.50

¹H NMR (400 MHz, DMSO-d6) δ ppm 9.31 (1H, s), 8.34 (1H, d, J=5.70 Hz), 7.83 (1H, s), 7.77 (1H, s), 7.65 (1H, d, J=2.19 Hz), 7.27 (1H, m), 6.98 (1H, m), 4.60 (3H, m), 4.13 (4H, m), 3.60 (2H, t, J=5.15 Hz), 3.21 (3H, s), 2.89 (3H, s), 2.38 (4H, m).

### Example 182: 1-(2-(6-(1-methyl-1H-pyrazol-4-yl) pyrazolo[1,5-a]pyridine-3-carbonyl)-2-azaspiro[3.3]heptan-6-yl)-3-(3-(trifluoromethyl)phenyl)urea

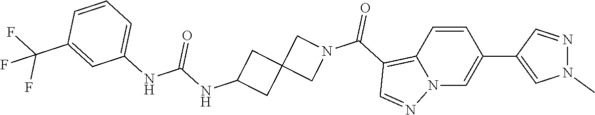 [SMILES: Cn1cc(-c2ccc3c(C(=O)N4CC5(CC(NC(=O)Nc6cccc(C(F)(F)F)c6)C5)C4)cnn3c2)cn1]

### Example 182

In a reactor, Intermediate 53 (0.100 g, 0.19 mmol), 1-methyl-4-(4,4,5,5-tetramethyl-1,3,2-dioxaborolan-2-yl)-1H-pyrazole (0.040 g, 0.19 mmol) and K₂CO₃ (0.066 g, 0.48 mmol) were placed and dioxane (0.55 mL) was added via syringe under argon atmosphere. Solution was filled with argon, next Pd(dppf)Cl₂ (0.014 g, 0.019 mmol) was added. The tube was sealed and heated overnight at 110° C. The reaction mixture was diluted with water and extracted with DCM. Organic layer was washed with water, brine, dried over Na₂SO₄ and concentrated on rotary evaporator. The crude material was purified via FCC (DCM/MeOH from 100/0 to 90/10) to give the title compound (20 mg, 0.038 mmol, 20% yield).

LCMS (ESI): Method 8, t_{R}=3.45 min, m/z (M+1)=524.23

¹H NMR (400 MHz, DMSO-d6) δ 9.10 (t, J=1.3 Hz, 1H), 8.75 (s, 1H), 8.27 (d, J=10.3 Hz, 2H), 8.17 (dd, J=9.1, 1.0 Hz, 1H), 8.03 (d, J=0.8 Hz, 1H), 7.94 (d, J=2.2 Hz, 1H), 7.75 (dd, J=9.2, 1.6 Hz, 1H), 7.51 (d, J=8.4 Hz, 1H), 7.44 (t, J=7.9 Hz, 1H), 7.22 (d, J=7.6 Hz, 1H), 6.58 (d, J=7.6 Hz, 1H), 4.48 (d, J=47.4 Hz, 2H), 4.06 (td, J=17.7, 17.3, 9.3 Hz, 3H), 2.59-2.52 (m, 2H), 2.15 (dd, J=12.0, 8.7 Hz, 2H).

### Example 192: 1-(2-(1-methyl-3-morpholino-1H-imidazo[1,2-b]pyrazole-7-carbonyl)-2-azaspiro[3.3]heptan-6-yl)-3-(3-(trifluoromethyl)phenyl)urea

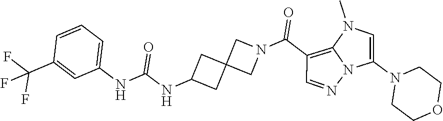 [SMILES: Cn1cc(N2CCOCC2)n2ncc(C(=O)N3CC4(CC(NC(=O)Nc5cccc(C(F)(F)F)c5)C4)C3)c12]

### Example 192

An ElectraSyn vial (5 mL) with a stir bar was charged with dibromonickel; 1-methoxy-2-(2-methoxyethoxy) ethane (84 mg, 0.24 mmol), 2,2′-bipyridine (111 mg, 0.71 mmol), TBAB (2.38 ml, 0.48 mmol), Intermediate 52 (250 mg, 0.48 mmol), morpholine (0.062 ml, 0.71 mmol), DBU (0.143 ml, 0.95 mmol) and DMA (6 mL). The vial was closed with an ElectraSyn 2.0 vial cap equipped with anode ((+) RVC, 47 mm×8 mm×2 mm) and cathode ((−) Ni foam, 0.8 cm×4.7×0.1 cm) inserted into the mixture. The vial was then evacuated and backfilled with an argon balloon. The reaction mixture was electrolyzed under a constant current of 4 mA until complete consumption of the starting material as judged by LC-MS. Then, the ElectraSyn vial cap was removed and electrodes were rinsed with a mixture of EtOAc:hexanes=1:1, which was combined with the crude mixture. Aqueous sat. NH₄Cl was then added to the combined solutions; the resulting solution was extracted with a mixture of EtOAc:hexanes=1:1. The combined organic layers were dried over anhydrous Na₂SO₄ and concentrated in vacuo. The crude material was purified by RF FCC to give the desired product (4 mg, 0.008 mmol, 2% yield).

LCMS (ESI): Method 5, t_{R}=5.82 min, m/z (M+1)=532.40

¹H NMR (600 MHz, DMSO-d6) δ ppm 8.88 (1H, br s), 7.94 (1H, s), 7.75 (1H, m), 7.51 (1H, m), 7.43 (1H, t, J=7.92 Hz), 7.21 (1H, br d, J=7.63 Hz), 6.69 (1H, br d, J=7.34 Hz), 6.62 (1H, d, J=1.03 Hz), 4.03 (2H, m), 3.89 (2H, s), 4.16 (4H, m), 3.76 (3H, m), 3.10 (3H, m), 2.52 (2H, m), 2.12 (2H, td, J=9.13, 2.71 Hz).

### Example 227: 1-(2-(6-(1-(2-methoxyethyl)-1H-pyrazol-4-yl) pyrazolo[1,5-a]pyridine-3-carbonyl)-2-azaspiro[3.3]heptan-6-yl)-1-methyl-3-(5-(trifluoromethyl)pyridin-3-yl)urea

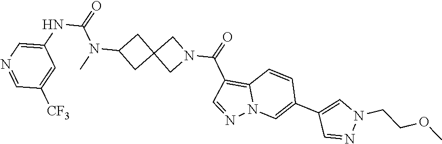 [SMILES: COCCn1cc(-c2ccc3c(C(=O)N4CC5(CC(N(C)C(=O)Nc6cncc(C(F)(F)F)c6)C5)C4)cnn3c2)cn1]

To a suspension of Intermediate 129 (70 mg, 0.13 mmol) and 1-(2-methoxyethyl)-4-(4,4,5,5-tetramethyl-1,3,2-dioxaborolan-2-yl) pyrazole (49.3 mg, 0.195 mmol) in Me-THF (1.5 mL), potassium phosphate tribasic (69 mg, 0.33 mmol) was added. After applying three N₂/vacuum cycles, tetrakis(triphenylphosphine) palladium (0) polymer bound (15.05 mg, 0.013 mmol) was added and the vial was sealed. The reaction was heated at 75° C. for 12 h. EtOAc and brine were added and the organic layer was concentrated under vacuum. The crude was purified by RF FCC to give title compound (28 mg, 0.05 mmol, 37% yield).

LCMS (ESI): Method 5, t_{R}=5.66 min, m/z (M+1)=583.50

¹H NMR (400 MHz, DMSO-d6) δ ppm 9.12 (1H, s), 8.94 (1H, d, J=2.41 Hz), 8.85 (1H, $), 8.52 (1H, s), 8.32 (1H, s), 8.31 (1H, t, J=1.97 Hz), 8.26 (1H, s), 8.18 (1H, d, J=9.17 Hz), 8.06 (1H, s), 7.77 (1H, dd, J=9.32, 1.43 Hz), 4.56 (3H, m), 4.29 (2H, t, J=5.26 Hz), 4.05 (2H, m), 3.72 (2H, t, J=5.26 Hz), 3.25 (3H, s), 2.93 (3H, s), 2.43 (4H, m)

The following Examples were prepared analogously to what described for Example 227, using XPhos Pd G3, starting from corresponding intermediate (see Table 42).

TABLE 42

| | | Product Amount |
|---|---|---|
| | Structure | (Yield) |
| Nº | Analytical Data | |
| Example 229 | 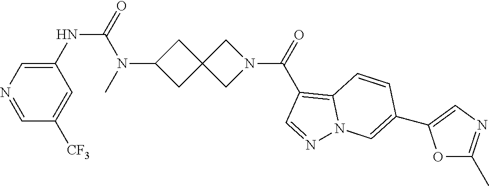 [SMILES: Cc1ncc(-c2ccc3c(C(=O)N4CC5(CC(N(C)C(=O)Nc6cncc(C(F)(F)F)c6)C5)C4)cnn3c2)o1] | 23 mg (33%) |
| | LCMS (ESI): Method 5, t_{R} = 5.82 min, m/z (M + 1) = 540.50 | |
| | ¹H NMR (400 MHZ, DMSO-d6) δ ppm 9.06 (1 H, s), 8.91 (1 H, d, J = 2.19 Hz), | |
| | 8.82 (1 H, s), 8.48 (1 H, s), 8.31 (1 H, br s), 8.27 (1 H, s), 8.22 (1 H, d, J = 9.21 | |
| | Hz), 7.76 (1 H, dd, J = 9.32, 1.43 Hz), 7.64 (1 H, s), 4.43 (1 H, m), 4.53 (3 H, m), | |
| | 4.08 (2 H, m), 2.89 (3 H, s), 2.47 (3 H, br s), 2.37 (1 H, m), 2.40 (4 H, m). | |
| Example 230 | 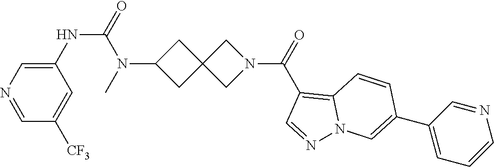 [SMILES: CN(C(=O)Nc1cncc(C(F)(F)F)c1)C1CC2(C1)CN(C(=O)c1cnn3cc(-c4cccnc4)ccc13)C2] | 35 mg (50%) |
| | LCMS (ESI): Method 5, t_{R} = 5.28 min, m/z (M + 1) = 536.40 | |
| | ¹H NMR (400 MHZ, DMSO-d6) δ ppm 9.30 (1 H, s), 9.04 (1 H, d, J = 2.41 Hz), | |
| | 8.94 (1 H, d, J = 2.19 Hz), 8.86 (1 H, s), 8.62 (1 H, dd, J = 4.71, 1.43 Hz), 8.51 (1 | |
| | H, s), 8.36 (1 H, br s), 8.31 (1 H, s), 8.29 (1 H, d, J = 9.21 Hz), 8.24 (1 H, dt, | |
| | J = 8.11, 1.97 Hz), 7.92 (1 H, dd, J = 9.21, 1.53 Hz), 7.54 (1 H, dd, J = 7.89, 4.82 | |
| | Hz), 4.58 (3 H, m), 4.09 (2 H, m), 2.93 (3 H, s), 2.44 (4 H, m). | |
| Example 233 | 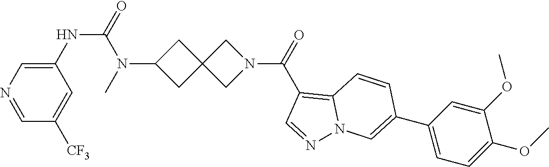 [SMILES: COc1ccc(-c2ccc3c(C(=O)N4CC5(CC(N(C)C(=O)Nc6cncc(C(F)(F)F)c6)C5)C4)cnn3c2)cc1OC] | 26 mg (34%) |
| | LCMS (ESI): Method 5, t_{R} = 6.58 min, m/z (M + 1) = 595.50 | |
| | ¹H NMR (400 MHZ, DMSO-d6) δ ppm 9.16 (1 H, s), 8.95 (1 H, d, J-2.19 Hz), | |
| | 8.86 (1 H, s), 8.52 (1 H, s), 8.31 (2 H, s), 8.23 (1 H, d, J = 9.21 Hz), 7.88 (1 H, | |
| | dd, J = 9.21, 1.53 Hz), 7.39 (1 H, d, J = 1.97 Hz), 7.34 (1 H, dd, J = 8.33, 2.19 Hz), | |
| | 7.07 (1 H, d, J = 8.33 Hz), 4.57 (3 H, m), 4.10 (2 H, m), 3.88 (3 H, s), 3.81 (3 H, | |
| | s), 2.93 (3 H, s), 2.44 (4 H, m). | |
| Example 234 | 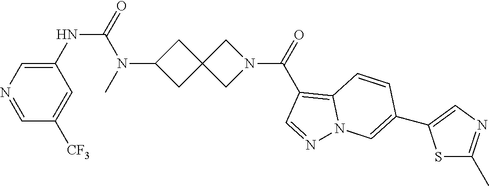 [SMILES: Cc1ncc(-c2ccc3c(C(=O)N4CC5(CC(N(C)C(=O)Nc6cncc(C(F)(F)F)c6)C5)C4)cnn3c2)s1] | 30 mg (41%) |
| | LCMS (ESI): Method 5, t_{R} = 6.15 min, m/z (M + 1) = 556.40 | |
| | ¹H NMR (600 MHz, DMSO-d6) δ ppm 9.17 (1 H, s), 8.94 (1 H, d, J = 2.20 Hz), | |
| | 8.86 (1 H, s), 8.52 (1 H, s), 8.34 (1 H, m), 8.31 (1 H, s), 8.23 (1 H, dd, J = 9.32, | |
| | 0.66 Hz), 8.19 (1 H, s), 7.77 (1 H, dd, J = 9.24, 1.61 Hz), 4.55 (3 H, m), 4.02 (2 | |
| | H, m), 2.93 (3 H, m), 2.71 (3 H, s), 2.43 (4 H, m). | |
| Example 236 | 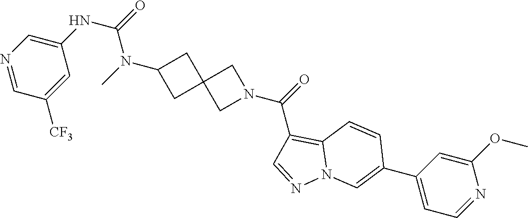 [SMILES: COc1cc(-c2ccc3c(C(=O)N4CC5(CC(N(C)C(=O)Nc6cncc(C(F)(F)F)c6)C5)C4)cnn3c2)ccn1] | 30 mg (41%) |
| | LCMS (ESI): Method 5, t_{R} = 6.28 min, m/z (M + 1) = 566.40 | |
| | ¹H NMR (600 MHz, DMSO-d6) δ ppm 9.39 (1 H, s), 8.94 (1 H, d, J = 2.20 Hz), | |
| | 8.86 (1 H, s), 8.52 (1 H, m), 8.38 (1 H, br s), 8.31 (1 H, m), 8.27 (2 H, m), 7.96 | |
| | (1 H, dd, J = 9.39, 1.61 Hz), 7.49 (1 H, dd, J = 5.43, 1.61 Hz), 7.31 (1 H, d, J = 1.03 | |
| | Hz), 4.48 (1 H, m), 4.58 (2 H, m), 4.09 (2 H, m), 3.91 (3 H, s), 2.93 (3 H, s), 2.44 (4 H, m). | |
| Example 237 | 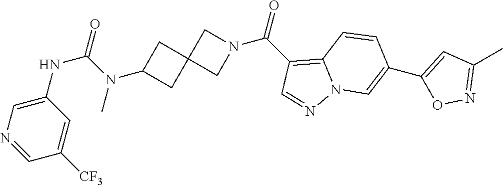 [SMILES: Cc1cc(-c2ccc3c(C(=O)N4CC5(CC(N(C)C(=O)Nc6cncc(C(F)(F)F)c6)C5)C4)cnn3c2)on1] | 12 mg (17%) |
| | LCMS (ESI): Method 5, t_{R} = 5.92 min, m/z (M + 1) = 540.40 | |
| | ¹H NMR (400 MHZ, DMSO-d6) δ ppm 9.40 (1 H, s), 8.94 (1 H, s), 8.85 (1 H, | |
| | s), 8.52 (1 H, s), 8.42 (1 H, br s), 8.30 (2 H, m), 7.89 (1 H, d, J = 9.31 Hz), 7.05 | |
| | (1 H, s), 4.57 (3 H, m), 4.11 (2 H, m), 2.93 (3 H, s), 2.44 (4 H, m), 2.32 (3 H, s). | |
| Example 241 | 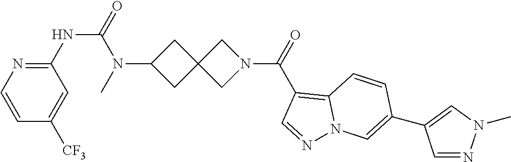 [SMILES: CN(C(=O)Nc1cc(C(F)(F)F)ccn1)C1CC2(C1)CN(C(=O)c1cnn3cc(-c4cnn(C)c4)ccc13)C2] | 13 mg (43%) |
| | LCMS (ESI): Method 5, t_{R} = 5.91 min, m/z (M + 1) = 539.40 | |
| | ¹H NMR (400 MHZ, DMSO-d6) δ ppm 9.43 (1 H, s), 9.10 (1 H, s), 8.50 (1 H, d, | |
| | J = 5.04 Hz), 8.29 (1 H, s), 8.25 (1 H, br s), 8.17 (1 H, d, J = 9.21 Hz), 8.14 (1 H, | |
| | s), 8.03 (1 H, s), 7.75 (1 H, d, J = 9.21 Hz), 7.31 (1 H, d, J = 5.26 Hz), 4.57 (3 H, | |
| | m), 4.08 (2 H, m), 3.88 (3 H, s), 2.91 (3 H. s), 2.41 (4 H. m). | |
| Example 242 | 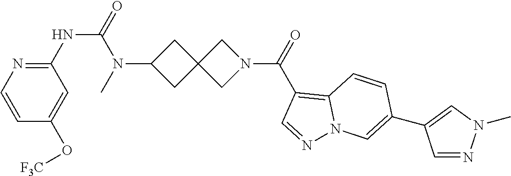 [SMILES: CN(C(=O)Nc1cc(OC(F)(F)F)ccn1)C1CC2(C1)CN(C(=O)c1cnn3cc(-c4cnn(C)c4)ccc13)C2] | 6 mg (17%) |
| | LCMS (ESI): Method 5, t_{R} = 6.03 min, m/z (M + 1) = 555.40 | |
| | ¹H NMR (400 MHz, DMSO-d6) δ ppm 9.32 (1 H, s), 9.10 (1 H, s), 8.34 (1 H, d, | |
| | J = 5.48 Hz), 8.29 (1 H, s), 8.25 (1 H, s), 8.18 (1 H, d, J = 9.21 Hz), 8.03 (1 H, s), | |
| | 7.84 (1 H, s), 7.75 (1 H, dd, J = 9.10, 1.21 Hz), 6.98 (1 H, m), 4.57 (3 H, m), 4.10 | |
| | (2 H, m), 3.88 (3 H, s), 2.90 (3 H, s), 2.41 (4 H, br dd, J = 13.04, 9.32 Hz). | |
| Example 243 | 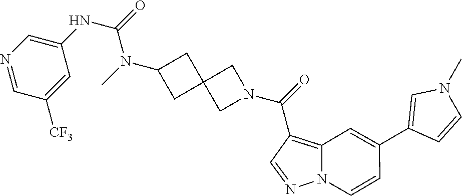 [SMILES: CN(C(=O)Nc1cncc(C(F)(F)F)c1)C1CC2(C1)CN(C(=O)c1cnn3ccc(-c4ccn(C)c4)cc13)C2] | 7 mg (14%) |
| | LCMS (ESI): Method 5, t_{R} = 5.49 min, m/z (M + 1) = 539.40 | |
| | ¹H NMR (400 MHZ, DMSO-d6) δ ppm 8.94 (1 H, s), 8.86 (1 H, s), 8.77 (1 H, d, | |
| | J = 7.23 Hz), 8.52 (1 H, s), 8.38 (1 H, s), 8.31 (1 H, br s), 8.26 (1 H, s), 8.24 (1 H, | |
| | s), 8.00 (1 H, s), 7.33 (1 H, br d, J = 7.23 Hz), 4.57 (3 H, m), 4.10 (2 H, m), 3.90 | |
| | (3 H, s), 2.93 (3 H, s), 2.42 (4 H, br d, J = 8.11 Hz). | |
| Example 245 | 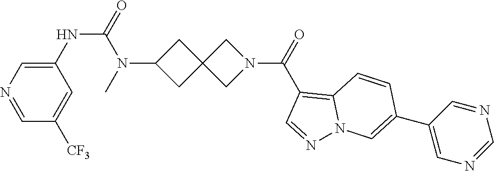 [SMILES: CN(C(=O)Nc1cncc(C(F)(F)F)c1)C1CC2(C1)CN(C(=O)c1cnn3cc(-c4cncnc4)ccc13)C2] | 12 mg (17%) |
| | LCMS (ESI): Method 5, t_{R} = 5.09 min, m/z (M + 1) = 537.30 | |
| | ¹H NMR (400 MHZ, DMSO-d6) δ ppm 9.44 (1 H, s), 9.29 (2 H, s), 9.23 (1 H, | |
| | s), 8.95 (1 H, d, J = 2.19 Hz), 8.86 (1 H, s), 8.52 (1 H, s), 8.39 (1 H, br s), 8.31 (2 H, m), | |
| | 7.98 (1 H, dd, J = 9.43, 1.53 Hz), 4.58 (3 H, m), 4.08 (2 H, m), 2.93 (3 H, s), 2.44 (4 H, m). | |
| Example 246 | 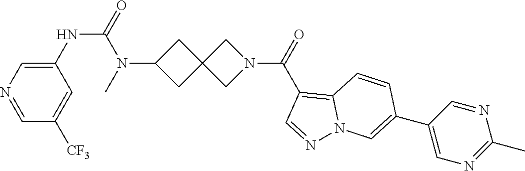 [SMILES: Cc1ncc(-c2ccc3c(C(=O)N4CC5(CC(N(C)C(=O)Nc6cncc(C(F)(F)F)c6)C5)C4)cnn3c2)cn1] | 15 mg (21%) |
| | LCMS (ESI): Method 5, t_{R} = 5.29 min, m/z (M + 1) = 551.30 | |
| | ¹H NMR (400 MHZ, DMSO-d6) δ ppm 9.39 (1 H, s), 9.16 (2 H, s), 8.95 (1 H, d, | |
| | J = 2.19 Hz), 8.86 (1 H, s), 8.52 (1 H, s), 8.38 (1 H, br s), 8.30 (2 H, m), 7.94 (1 | |
| | H, dd, J = 9.21, 1.53 Hz), 4.58 (3 H, m), 4.12 (2 H, m), 2.93 (3 H, s), 2.69 (3 H, | |
| | s), 2.44 (4 H, m). | |
| Example 249 | 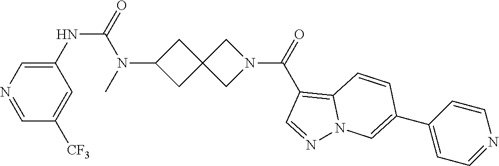 [SMILES: CN(C(=O)Nc1cncc(C(F)(F)F)c1)C1CC2(C1)CN(C(=O)c1cnn3cc(-c4ccncc4)ccc13)C2] | 10 mg (14%) |
| | LCMS (ESI): Method 5, t_{R} = 4.30 min, m/z (M + 1) = 536.21 | |
| | ¹H NMR (400 MHZ, DMSO-d6) δ ppm 9.42 (1 H, s), 8.95 (1 H, d, J = 1.97 Hz), | |
| | 8.86 (1 H, s), 8.68 (2 H, d, J = 6.14 Hz), 8.52 (1 H, s), 8.39 (1 H, br s), 8.30 (2 H, | |
| | d, J = 8.99 Hz), 7.98 (1 H, dd, J = 9.43, 1.53 Hz), 7.88 (2 H, d, J = 4.98 Hz), 4.59 (2 | |
| | H, m), 4.47 (1 H, br s), 4.17 (1 H, br s), 4.04 (1 H, br s), 2.93 (3 H, s), 2.44 (4 H, m). | |
| Example 253 | 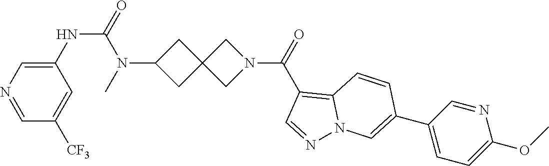 [SMILES: COc1ccc(-c2ccc3c(C(=O)N4CC5(CC(N(C)C(=O)Nc6cncc(C(F)(F)F)c6)C5)C4)cnn3c2)cn1] | 22 mg (30%) |
| | LCMS (ESI): Method 5, t_{R} = 5.77 min, m/z (M + 1) = 566.28 | |
| | ¹H NMR (400 MHZ, DMSO-d6) δ ppm 9.20 (1 H, s), 8.95 (1 H, m), 8.86 (1 H, | |
| | s), 8.63 (1 H, d, J = 2.41 Hz), 8.52 (1 H, s), 8.33 (1 H, br s), 8.31 (1 H, s), 8.26 (1 | |
| | H, d, J = 9.21 Hz), 8.17 (1 H, dd, J = 8.77, 2.63 Hz), 7.86 (1 H, d, J = 9.19 Hz), 6.96 | |
| | (1 H, d, J = 8.55 Hz), 4.59 (2 H, quin, J = 8.66 Hz), 4.45 (1 H, br d, J = 5.48 Hz), | |
| | 4.17 (1 H, m), 4.03 (1 H, m), 3.91 (3 H, s), 2.93 (3 H, s), 2.44 (4 H, m). | |
| Example 258 | 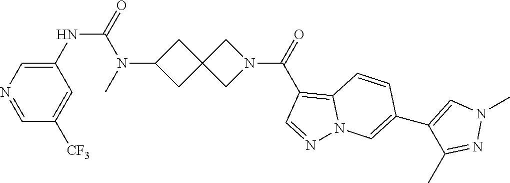 [SMILES: Cc1nn(C)cc1-c1ccc2c(C(=O)N3CC4(CC(N(C)C(=O)Nc5cncc(C(F)(F)F)c5)C4)C3)cnn2c1] | 15 mg (21%) |
| | LCMS (ESI): Method 5, t_{R} = 5.59 min, m/z (M + 1) = 552.97 | |
| | ¹H NMR (400 MHZ, DMSO-d6) δ ppm 8.94 (1 H, s), 8.85 (1 H, s), 8.77 (1 H, | |
| | s), 8.52 (1 H, s), 8.31 (1 H, s), 8.27 (1 H, br s), 8.19 (1 H, d, J = 9.21 Hz), 8.04 (1 | |
| | H, s), 7.60 (1 H, d, J-9.21 Hz), 4.58 (2 H, m), 4.44 (1 H, m), 4.15 (1 H, br s), | |
| | 4.02 (1 H, br s), 3.81 (3 H, s), 2.93 (3 H, s), 2.43 (4 H, m), 2.34 (3 H, s). | |
| Example 261 | 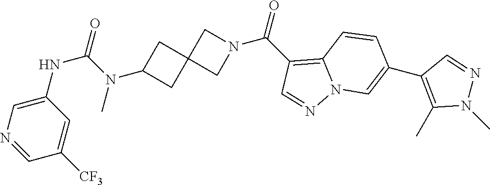 [SMILES: Cc1c(-c2ccc3c(C(=O)N4CC5(CC(N(C)C(=O)Nc6cncc(C(F)(F)F)c6)C5)C4)cnn3c2)cnn1C] | 21 mg (29%) |

LCMS (ESI): Method 5, t_{R} = 5.55 min, m/z (M + 1) = 553
¹H NMR (400 MHZ, DMSO-d6) δ ppm 8.95 (1 H, s), 8.86 (1 H, s), 8.78 (1 H,
s), 8.52 (1 H, s), 8.31 (1 H, br s), 8.28 (1 H, br s), 8.21 (1 H, d, J = 9.21 Hz), 7.71
(1 H, s), 7.60 (1 H, d, J = 9.43 Hz), 4.58 (2 H, m), 4.44 (1 H, br d, J = 2.19 Hz),
4.15 (1 H, br s), 4.02 (1 H, br s), 3.81 (3 H, s), 2.93 (3 H, s), 2.42 (7 H, m).

The following compounds of formula (I) were prepared by applying the experimental conditions described above:
1-(4-((4-methylpiperazin-1-yl)methyl)-3-(trifluoromethyl)phenyl)-3-(2-(pyrazolo[5,1-b]thiazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(4-(2-morpholinoethoxy)-3-(trifluoromethyl)phenyl)urea;
1-(2-(6,7-dihydro-5H-pyrrolo[1,2-a]imidazole-3-carbonyl)-2-azaspiro[3.3]heptan-6-yl)-3-(3-(trifluoromethyl)phenyl)urea;
1-(3-fluorophenyl)-3-(2-(pyrazolo[5,1-b]thiazole-7-carbonyl)-2-azaspiro[3.3]heptan-6-yl)urea;
1-(2-(2,3-dihydro-1H-imidazo[1,2-b]pyrazole-7-carbonyl)-2-azaspiro[3.3]heptan-6-yl)-3-(3-(trifluoromethyl)phenyl)urea;
1-(2-(1-methyl-2,3-dihydro-1H-imidazo[1,2-b]pyrazole-7-carbonyl)-2-azaspiro[3.3]heptan-6-yl)-3-(3-(trifluoromethyl)phenyl)urea;
1-(3-fluorophenyl)-3-(2-(1-methyl-1H-imidazo[1,2-b]pyrazole-7-carbonyl)-2-azaspiro[3.3]heptan-6-yl)urea;
1-(2-(pyrazolo[1,5-a]pyrimidine-3-carbonyl)-2-azaspiro[3.3]heptan-6-yl)-3-(3-(trifluoro-methyl)phenyl)urea;
1-(2-(pyrazolo[1,5-a]pyrimidine-7-carbonyl)-2-azaspiro[3.3]heptan-6-yl)-3-(3-(trifluoro-methyl)phenyl)urea;
1-(3-(tert-butyl)-1-methyl-1H-pyrazol-5-yl)-3-(2-(1-methyl-1H-imidazo[1,2-b]pyrazole-7-carbonyl)-2-azaspiro[3.3]heptan-6-yl)urea;
1-(5-(tert-butyl)-1-methyl-1H-pyrazol-3-yl)-3-(2-(1-methyl-1H-imidazo[1,2-b]pyrazole-7-carbonyl)-2-azaspiro[3.3]heptan-6-yl)urea;
1-(5-cyclopropylpyridin-3-yl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(2-(trifluoromethyl)pyridin-4-yl)urea;
1-(5-cyanopyridin-3-yl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(4-(trifluoromethyl)pyrimidin-2-yl)urea;
1-(2-(5-oxo-4,5-dihydropyrazolo[1,5-a]pyrimidine-3-carbonyl)-2-azaspiro[3.3]heptan-6-yl)-3-(3-(trifluoromethyl)phenyl)urea;
1-(3-(tert-butyl)-1-(pyridin-3-yl)-1H-pyrazol-5-yl)-3-(2-(1-methyl-1H-imidazo[1,2-b]pyrazole-7-carbonyl)-2-azaspiro[3.3]heptan-6-yl)urea;
1-(2-(1-(2-(dimethylamino)ethyl)-1H-imidazo[1,2-b]pyrazole-7-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(3-(4-methyl-1H-imidazol-1-yl)-5-(trifluoromethyl)phenyl)urea;
1-(2-(1-methyl-1H-imidazo[1,2-b]pyrazole-7-carbonyl)-2-azaspiro[3.3]heptan-6-yl)-3-(3-(methylsulfonyl)-5-(trifluoromethyl)phenyl)urea;
1-(2-((1-acetylazetidin-3-yl)oxy)-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-((1-acetylpiperidin-4-yl)oxy)-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-(pyrazolo[1,5-a]pyrazin-3-yl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(3-(2,2,2-trifluoro-1-hydroxyethyl)phenyl)urea;
1-(2-(2,3-dihydropyrazolo[5,1-b]oxazole-7-carbonyl)-2-azaspiro[3.3]heptan-6-yl)-3-(3-(trifluoromethyl)phenyl)urea;
1-(2-(imidazo[2,1-b]thiazole-2-carbonyl)-2-azaspiro[3.3]heptan-6-yl)-3-(3-(trifluoromethyl)phenyl)urea;
1-(2-(7-methylthieno[3,2-d]pyrimidine-4-carbonyl)-2-azaspiro[3.3]heptan-6-yl)-3-(3-(trifluoromethyl)phenyl)urea;
1-(2-(1-methyl-1H-pyrrolo[2,3-c]pyridine-3-carbonyl)-2-azaspiro[3.3]heptan-6-yl)-3-(3-(trifluoromethyl)phenyl)urea;
1-(2-(pyrazolo[1,5-a]pyrimidin-6-yl)-2-azaspiro[3.3]heptan-6-yl)-3-(3-(trifluoromethyl)phenyl)urea;
1-(2-(pyrazolo[1,5-a]pyridin-3-yl)-2-azaspiro[3.3]heptan-6-yl)-3-(3-(trifluoromethyl)phenyl)urea;
1-(2-(thieno[3,2-b]pyridin-6-yl)-2-azaspiro[3.3]heptan-6-yl)-3-(3-(trifluoromethyl)phenyl)urea;
1-(2-(imidazo[2,1-b]thiazole-6-carbonyl)-2-azaspiro[3.3]heptan-6-yl)-3-(3-(trifluoromethyl)phenyl)urea;
1-(5-isopropylpyridin-3-yl)-3-(2-(1-methyl-1H-imidazo[1,2-b]pyrazole-7-carbonyl)-2-azaspiro[3.3]heptan-6-yl)urea;
1-(cyclopropylmethyl)-1-(2-(1-methyl-1H-imidazo[1,2-b]pyrazole-7-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(6-(trifluoromethyl) pyrazin-2-yl)urea;
1-(5-(difluoromethyl)pyridin-3-yl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(3-(S-(trifluoromethyl) sulfonimidoyl)phenyl)urea;
1-(5-(tert-butyl)pyridin-3-yl)-3-(2-(1-methyl-1H-imidazo[1,2-b]pyrazole-7-carbonyl)-2-azaspiro[3.3]heptan-6-yl)urea;
1-(3-(hydroxymethyl)-2-methoxy-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-3-(2-methyl-5-(trifluoromethyl)pyridin-3-yl)urea;
1-methyl-3-(2-methyl-5-(trifluoromethyl)pyridin-3-yl)-1-(2-(pyrazolo[5,1-b]thiazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(2-methyl-5-(trifluoromethyl)pyridin-3-yl)urea;
1-isobutyl-1-(2-(1-methyl-1H-imidazo[1,2-b]pyrazole-7-carbonyl)-2-azaspiro[3.3]heptan-6-yl)-3-(3-(trifluoromethyl)phenyl)urea;
1-methyl-1-(2-(pyrazolo[1,5-a]pyrazine-3-carbonyl)-2-azaspiro[3.3]heptan-6-yl)-3-(6-(trifluoromethyl)pyrimidin-4-yl)urea;
1-(5-(1,1-difluoroethyl)pyridin-3-yl)-3-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-1-methyl-3-(5-(trifluoromethyl)pyridin-3-yl)urea;
1-(2-(1H-pyrazolo[3,4-b]pyridine-4-carbonyl)-2-azaspiro[3.3]heptan-6-yl)-1-methyl-3-(6-(trifluoromethyl)pyrimidin-4-yl)urea;
1-methyl-1-(2-(1-methyl-1H-imidazo[1,2-b]pyrazole-7-carbonyl)-2-azaspiro[3.3]heptan-6-yl)-3-(5-(trifluoromethoxy)pyridin-2-yl)urea;
1-methyl-1-(2-(1-methyl-1H-imidazo[1,2-b]pyrazole-7-carbonyl)-2-azaspiro[3.3]heptan-6-yl)-3-(6-(trifluoromethoxy)pyridin-3-yl)urea;
1-(2-(1-methyl-1H-imidazo[1,2-b]pyrazole-7-carbonyl)-2-azaspiro[3.3]heptan-6-yl)-3-(5-(trifluoromethoxy)pyridin-2-yl)urea;
1-(2-(1-methyl-1H-imidazo[1,2-b]pyrazole-7-carbonyl)-2-azaspiro[3.3]heptan-6-yl)-3-(6-(trifluoromethoxy)pyridin-3-yl)urea;
1-(2-(1-(cyanomethyl)-1H-imidazo[1,2-b]pyrazole-7-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(6-(trifluoromethyl)pyridazin-4-yl)urea;
1-methyl-3-(2-methyl-5-(trifluoromethoxy)phenyl)-1-(2-(pyrazolo[1,5-a]pyrazine-3-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-1-methyl-3-(5-(trifluoromethoxy)pyridin-3-yl)urea;
1-(6-methyl-2-(1-methyl-1H-imidazo[1,2-b]pyrazole-7-carbonyl)-2-azaspiro[3.3]heptan-6-yl)-3-(3-(trifluoromethyl)phenyl)urea;
1-(2-(1H-pyrazolo[3,4-b]pyridine-4-carbonyl)-2-azaspiro[3.3]heptan-6-yl)-1-methyl-3-(4-(trifluoromethyl)pyridin-2-yl)urea;
1-methyl-1-(2-(pyrazolo[1,5-a]pyridine-3-carbonyl)-2-azaspiro[3.3]heptan-6-yl)-3-(6-(trifluoromethyl)pyrimidin-4-yl)urea;
1-methyl-1-(2-(1-methyl-1H-imidazo[1,2-b]pyrazole-7-carbonyl)-2-azaspiro[3.3]heptan-6-yl)-3-(1-methyl-2-oxo-5-(trifluoromethyl)-1,2-dihydropyridin-3-yl)urea;
1-(2-(1-((methylsulfonyl)methyl)-1H-imidazo[1,2-b]pyrazole-7-carbonyl)-2-azaspiro[3.3]heptan-6-yl)-3-(3-(trifluoromethyl)phenyl)urea;
1-(2-(1-(2-hydroxyethyl)-1H-imidazo[1,2-b]pyrazole-7-carbonyl)-2-azaspiro[3.3]heptan-6-yl)-1-methyl-3-(3-(trifluoromethyl)phenyl)urea;
1-(2-(1-((2-methoxyethoxy)methyl)-1H-imidazo[1,2-b]pyrazole-7-carbonyl)-2-azaspiro[3.3]heptan-6-yl)-1-methyl-3-(5-(trifluoromethyl)pyridin-3-yl)urea;
3-(2-methoxy-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(6-aminopyrazolo[1,5-a]pyridine-3-carbonyl)-2-azaspiro[3.3]heptan-6-yl)-1-methyl-3-(5-(trifluoromethyl)pyridin-3-yl)urea;
3-(3-cyclopropylisoxazol-5-yl)-1-methyl-1-(2-(1-methyl-1H-imidazo[1,2-b]pyrazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(3-(trifluoromethyl) isoxazol-5-yl)urea;
1-methyl-1-(2-(thieno[3,2-d]pyrimidine-4-carbonyl)-2-azaspiro[3.3]heptan-6-yl)-3-(5-(1,1,1-trifluoro-2-methylpropan-2-yl) isoxazol-3-yl)urea;
1-methyl-1-(2-(pyrazolo[1,5-a]pyridine-3-carbonyl)-2-azaspiro[3.3]heptan-6-yl)-3-(5-(1,1,1-trifluoro-2-methylpropan-2-yl) isoxazol-3-yl)urea;
1-methyl-3-(1-methyl-2-oxo-5-(trifluoromethyl)-1,2-dihydropyridin-3-yl)-1-(2-(pyrazolo[5,1-b]thiazole-7-carbonyl)-2-azaspiro[3.3]heptan-6-yl)urea;
3-(3-fluoro-4-(trifluoromethyl)pyridin-2-yl)-1-methyl-1-(2-(1-methyl-1H-imidazo[1,2-b]pyrazole-7-carbonyl)-2-azaspiro[3.3]heptan-6-yl)urea;
1-methyl-1-(2-(thieno[3,2-d]pyrimidine-4-carbonyl)-2-azaspiro[3.3]heptan-6-yl)-3-(6-(trifluoromethyl)pyrimidin-4-yl)urea;
1-(2-(7H-pyrrolo[2,3-d]pyrimidine-4-carbonyl)-2-azaspiro[3.3]heptan-6-yl)-3-(4-bromopyridin-2-yl)-1-methylurea;
1-methyl-1-(2-(1-methyl-1H-imidazo[1,2-b]pyrazole-7-carbonyl)-2-azaspiro[3.3]heptan-6-yl)-3-(1-methyl-5-(1,1,1-trifluoro-2-methylpropan-2-yl)-1H-pyrazol-3-yl)urea;
2-(2-(1-methyl-1H-imidazo[1,2-b]pyrazole-7-carbonyl)-2-azaspiro[3.3]heptan-6-yl)-N-(6-(trifluoromethyl)pyrimidin-4-yl) acetamide;
2-(2-(pyrazolo[5,1-b]thiazole-7-carbonyl)-2-azaspiro[3.3]heptan-6-yl)-N-(4-(trifluoromethoxy)pyridin-2-yl) propanamide;
2-(2-(1-methyl-1H-imidazo[1,2-b]pyrazole-7-carbonyl)-2-azaspiro[3.3]heptan-6-yl)-N-(4-(trifluoromethoxy)pyridin-2-yl) propanamide;
2-methyl-2-(2-(1-methyl-1H-imidazo[1,2-b]pyrazole-7-carbonyl)-2-azaspiro[3.3]heptan-6-yl)-N-(4-(trifluoromethoxy)pyridin-2-yl) propanamide;
1-methyl-1-(2-(1-methyl-1H-imidazo[1,2-b]pyrazole-7-carbonyl)-2-azaspiro[3.3]heptan-6-yl)-3-(1-methyl-5-(1,1,1-trifluoro-2-methylpropan-2-yl)-1H-pyrazol-3-yl)urea;
1-methyl-1-(2-(4,5,6,7-tetrahydropyrazolo[1,5-a]pyridine-3-carbonyl)-2-azaspiro[3.3]heptan-6-yl)-3-(4-(trifluoromethyl)pyridin-2-yl)urea;
1-(2-(5-fluoro-1H-pyrrolo[2,3-b]pyridine-4-carbonyl)-2-azaspiro[3.3]heptan-6-yl)-1-methyl-3-(3-(trifluoromethyl)phenyl)urea;
3-(5-bromopyridazin-3-yl)-1-methyl-1-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridin-5-yl)-2-azaspiro[3.3]heptan-6-yl)-3-(4-bromopyridin-2-yl)urea;
1-(2-(1H-pyrazolo[3,4-b]pyridin-5-yl)-2-azaspiro[3.3]heptan-6-yl)-3-(6-(trifluoromethyl)pyrimidin-4-yl)urea;
1-(2-(7H-pyrrolo[2,3-d]pyrimidine-4-carbonyl)-2-azaspiro[3.3]heptan-6-yl)-1-methyl-3-(4-(trifluoromethyl)pyridin-2-yl)urea;
1-methyl-3-(2-methyl-6-(trifluoromethyl)pyrimidin-4-yl)-1-(2-(thieno[3,2-d]pyrimidine-4-carbonyl)-2-azaspiro[3.3]heptan-6-yl)urea;
1-methyl-3-(5-(perfluoroethyl)pyridazin-3-yl)-1-(2-(pyrazolo[5,1-b]thiazole-7-carbonyl)-2-azaspiro[3.3]heptan-6-yl)urea and
1-methyl-1-(2-(thieno[3,2-d]pyrimidine-4-carbonyl)-2-azaspiro[3.3]heptan-6-yl)-3-(5-(trifluoromethyl)pyridazin-3-yl)urea.

Comparative newly synthesized compounds C1, C2 and C3 were prepared as described below.

C1 is characterized by having a —CO—NH— linker replacing the —NH—CO-L1- linker of the compounds of the invention.

C2 is characterized by having a —CH₂—CO—NH— linker replacing the —NH—CO-L1- linker of the compounds of the invention.

C3 is characterized by having a structure XXIII

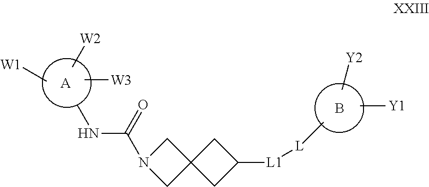

wherein the central core

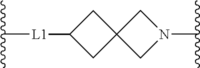

of the compounds of formula (I) has been flipped (for Rs being hydrogen).

### Compound C1: N-(2-(pyrazolo[1,5-a]pyrazine-3-carbonyl)-2-azaspiro[3.3]heptan-6-yl)-3-(trifluoromethyl)benzamide

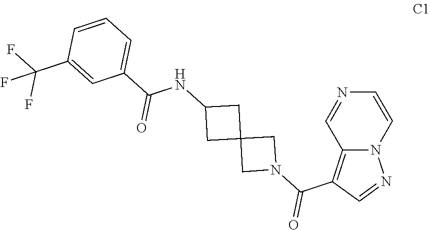 [SMILES: O=C(NC1CC2(C1)CN(C(=O)c1cnn3ccncc13)C2)c1cccc(C(F)(F)F)c1]

Compound C1 was prepared by following the procedure described for the synthesis of Example 1, step 3.

SM: Intermediate 10: 50 mg (1 eq); 3-(trifluoromethyl)benzoic acid: 37 mg (1 eq)

Amount/yield: 23 mg, 0.0544 mmol, 28%

LCMS (ESI): Method 3, t_{R} 3.73 min, m/z (M+1)=430.2

¹H NMR (400 MHz, DMSO-d6) δ 9.55 (d, J=1.5 Hz, 1H), 8.92-8.87 (m, 2H), 8.46 (s, 1H), 8.19 (s, 1H), 8.15 (d, J=8.1 Hz, 1H), 8.11 (d, J=4.8 Hz, 1H), 7.91 (d, J=7.6 Hz, 1H), 7.72 (t, J=7.7 Hz, 1H), 4.56 (d, J=52.8 Hz, 1H), 4.38-4.33 (m, 1H), 4.13 (d, J=50.2 Hz, 1H), 2.68-2.58 (m, 2H), 2.39-2.31 (m, 2H).

Compound C2 (N-(2-(pyrazolo[1,5-a]pyrazine-3-carbonyl)-2-azaspiro[3.3]heptan-6-yl)-2-(3-(trifluoromethyl)phenyl)acetamide) was prepared analogously to what described for Compound C1 by starting from commercially available 2-(3-(trifluoromethyl)phenyl) acetic acid (see Table 43).

TABLE 43

| | | Product Amount |
|---|---|---|
| | Structure | (Yield) |
| Nº | Analytical Data | |
| C2 | 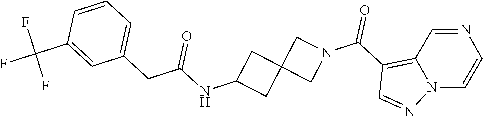 [SMILES: O=C(Cc1cccc(C(F)(F)F)c1)NC1CC2(C1)CN(C(=O)c1cnn3ccncc13)C2] | 20 mg, (24%) |

LCMS (ESI): Method 2, t_{R} = 3.8 min, m/z (M + 1) = 444.2
¹H NMR (400 MHZ, DMSO-d6) δ 9.60 (d, J = 1.5 Hz, 1H), 8.96 (dd, J = 1.5,
4.5 Hz, 1H), 8.49-8.47 (m, 2H), 8.16 (d, J-4.5 Hz, 1H), 7.67-7.58 (m,
4H), 4.61 (s, 1H), 4.51 (s, 1H), 4.22-4.14 (m, 2H), 4.08 (s, 1H), 3.55 (s,
2H), 2.62-2.58 (m, 2H), 2.23-2.12 (m, 2H).

### Compound C3: N-(2-((3-(trifluoromethyl)phenyl)carbamoyl)-2-azaspiro[3.3]heptan-6-yl) pyrazolo[1,5-a]pyrazine-3-carboxamide (Compound of Formula XX, Wherein L is CO and L1 is NR, Wherein R is Hydrogen)

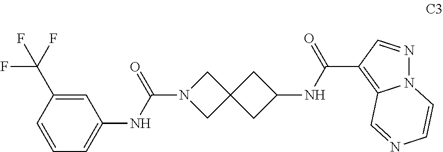 [SMILES: O=C(NC1CC2(C1)CN(C(=O)Nc1cccc(C(F)(F)F)c1)C2)c1cnn2ccncc12]

### Step 1-tert-butyl(2-((3-(trifluoromethyl)phenyl)carbamoyl)-2-azaspiro[3.3]heptan-6-yl)carbamate (Intermediate 18)

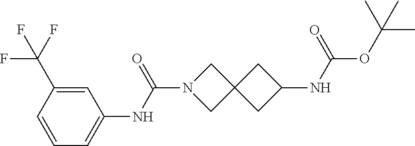 [SMILES: CC(C)(C)OC(=O)NC1CC2(C1)CN(C(=O)Nc1cccc(C(F)(F)F)c1)C2]

Intermediate 18 was prepared by following the same procedure reported for the synthesis of Intermediate 6.

SM: tert-butyl(2-azaspiro[3.3]heptan-6-yl)carbamate: 200 mg (1 eq); 1-isocyanato-3-(trifluoromethyl)benzene (1 eq)

Amount/yield: 376 mg, 0.941 mmol, 100%

LCMS (ESI): Method 4, t_{R}=1.67 min, m/z (M+1)=400.2

### Step 2—6-amino-N-(3-(trifluoromethyl)phenyl)-2-azaspiro[3.3]heptane-2-carboxamide (Intermediate 19)

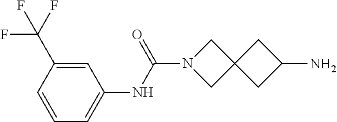 [SMILES: NC1CC2(C1)CN(C(=O)Nc1cccc(C(F)(F)F)c1)C2]

Intermediate 19 was prepared by following the same procedure reported for the synthesis of Intermediate 7.

SM: Intermediate 18: 376 mg (1 eq)

TFA: 1.4 mL (20 eq)

Amount/yield: 270 mg, 0.902 mmol, 96%

LCMS (ESI): Method 4, t_{R}=1.34 min, m/z (M+1)=300

### Step 3—N-(2-((3-(trifluoromethyl)phenyl)carbamoyl)-2-azaspiro[3.3]heptan-6-yl) pyrazolo[1,5-a]pyrazine-3-carboxamide (Compound C3)

Compound C3 was prepared by following the procedure described for the synthesis of Example 1, step 3.

SM: Intermediate 19: 100 mg (1 eq); pyrazolo[1,5-a]pyrazine-3-carboxylic acid: 55 mg (1 eq)

Amount/yield: 34 mg, 0.0755 mmol, 23%

LCMS (ESI): Method 3, t_{R}=3.82 min, m/z (M+1)=445.2

¹H NMR (400 MHz, DMSO-d6) δ 9.55 (d, J=1.3 Hz, 1H), 8.87 (dd, J=1.4, 4.7 Hz, 1H), 8.75 (s, 1H), 8.66-8.59 (m, 2H), 8.07 (d, J=4.8 Hz, 1H), 7.96 (s, 1H), 7.78-7.74 (m, 1H), 7.46 (t, J=8.0 Hz, 1H), 7.27-7.23 (m, 1H), 4.40-4.32 (m, 1H), 4.08 (s, 2H), 3.97 (s, 2H), 2.62-2.55 (m, 2H), 2.33-2.26 (m, 2H).

### PHARMACOLOGICAL ACTIVITY OF THE COMPOUNDS OF THE INVENTION

### In Vitro Assays

### Binding Assays

DDR1 and DDR2 binding assays were performed using Life Technologies LanthaScreen™ Europium Kinase Binding assay. The compounds were incubated with 5 nM DDR1 (Carna Biosciences) or 5 nM DDR2 (Life Technologies) for 1 h at rt in white 384-well OptiPlate (PerkinElmer), containing 20 nM or 10 nM Kinase Tracer 178 respectively and 2 nM Europium labelled anti-GST antibody (Life Technologies) in assay buffer (50 mM HEPES pH 7.5, 10 mM MgCl₂, 1 mM EGTA and 0.01% BRIJ35). The ratio of fluorescence emission 665 nm/615 nm after excitation at 340 nm was obtained using the Tecan Spark 20M plate reader. IC₅₀ values were determined in GraphPad Prism 7.0 software, using 4 parameter model: log (inhibitor) vs. response. IC₅₀ values were converted in Ki using the Cheng-Prusoff equation (Ki-IC₅₀/(1+ [Tracer]/Kd).

The results for representative compounds of the invention are provided in Table 44, wherein the compounds are classified in term of potency (Ki, nM) in binding with respect to their inhibitory activity on DDR1 and DDR2:

TABLE 44

| | Ki | Ki |
|---|---|---|
| | DDR1 | DDR2 |
| Example No. | (nM) | (nM) |
| 3, 44, 192 | + | + |
| 18, 25, 175, 176, 190, 208, 212, 213, 215, 220, 222, | ++ | + |
| 246, 290, 307,310 | | |
| 33, 170 | ++ | − |
| 7, 11, 12, 160, 163, 189, 240 | ++ | ++ |
| 78, 164, 173, 177, 178, 202, 211, 216, 225, 228, 250, | +++ | + |
| 259 | | |
| 1, 2, 5, 6, 8, 16, 17, 20, 22, 24, 26, 28, 29, 32, 35, 36, | +++ | ++ |
| 37, 38, 41, 42, 43, 47, 48, 50, 51, 52, 53, 54, 55, 56, | | |
| 57, 58, 59, 61, 62, 63, 64, 65, 66, 67, 68, 69, 72, 73, | | |
| 75, 77, 80, 81, 82, 83, 84, 85, 86, 87, 88, 91, 94, 95, | | |
| 100, 102, 103, 104, 105, 107, 108, 112, 114, 115, | | |
| 116, 121, 123, 126, 128, 129, 130, 131, 132, 133, | | |
| 134, 135, 136, 144, 145, 146, 150, 151, 152, 154, | | |
| 155, 156, 157, 158, 159, 161, 165, 166, 167, 168, | | |
| 169, 171, 172, 174, 174, 179, 180, 181, 183, 186, | | |
| 187, 188, 191, 193, 194, 195, 196, 197, 199, 200, | | |
| 201, 203, 204, 205, 206, 207, 209, 214, 217, 218, | | |
| 219, 221, 223, 224, 226, 227, 229, 230, 231, 232, | | |
| 233, 234, 235, 236, 237, 238, 239, 241, 242, 243, | | |
| 244, 245, 247, 248, 249, 251, 252, 253, 254, 255, | | |
| 256, 257, 258, 260, 261, 262, 263, 264, 266, 268, | | |
| 269, 274, 277, 278, 281, 283, 284, 286, 287, 288, | | |
| 289, 291, 292, 296, 297, 300, 303, 304, 305, 306, | | |
| 309, 311, 312 | | |
| 4, 9, 15, 19, 21, 23, 27, 30, 31, 34, 39, 40, 45, 46, | +++ | +++ |
| 49, 60, 70, 71, 74, 76, 79, 89, 90, 92, 93, 96, 97, | | |
| 98, 99, 101, 106, 109, 110, 111, 113, 117, 118, | | |
| 119, 120, 122, 124, 125, 127, 137, 138, 139, 140, | | |
| 141, 142, 143, 147, 148, 149, 153, 162, 182, 184, | | |
| 185, 198, 210, 265, 267, 270, 271, 272, 273, 275, | | |
| 276, 279, 280, 282, 285, 293, 294, 295, 298, 299, | | |
| 301, 302, 308 | | |

−: Ki higher than 100 nM
+: Ki between 25 and 100 nM
++: Ki between 5 nM and 25 nM
+++: Ki lower than 5 nM

As it can be appreciated, the compounds of Table 44, i.e. compounds according to the invention, show a good activity as antagonist of DDR1 and DDR2. Accordingly, the compounds of the invention can be effectively used for treating diseases, disorders or conditions associated with DDR receptors, such as fibrosis, e.g. pulmonary fibrosis, idiopathic pulmonary fibrosis (IPF), hepatic fibrosis, progressive pulmonary fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

### Comparative Compounds

Compounds C1, C2 and C3 were tested in the same binding assay described above and their Ki (nM) is reported in Table 45.

TABLE 45

| Example No | DDR1 Ki (nM) | DDR2 Ki (nM) |
|---|---|---|
| C1 | >500 | >500 |
| C2 | >500 | >500 |
| C3 | >500 | >500 |

The compounds of the present invention whose activity is reported in Table 44 have a binding affinity for DDR1 and DDR2 receptors, expressed as Ki, which is lower than 100 nM, in several cases lower than 25 nM or even lower than 5 nM. To the contrary, as it can be seen in Table 45, the comparative compounds C1, C2 and C3 did not show a binding affinity for DDR1 and DDR2 receptors, being their Ki over the detectable limit of the binding assay.

Thus, the presence of the —NH—CO-L1- linker, wherein L1 is NR, CH₂ or O, of the linker L, being —CO—, —CH₂— or absent, and of the central core in the compounds of formula (I) of the present invention unexpectedly and noteworthy determine a remarkable inhibitory activity on the DDR1 and DDR2 receptors.

1. A compound of formula (I) 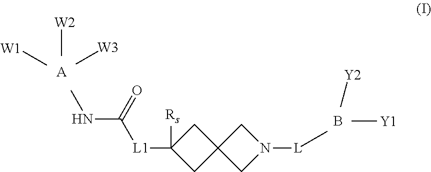
wherein
A is a ring selected from the group consisting of: 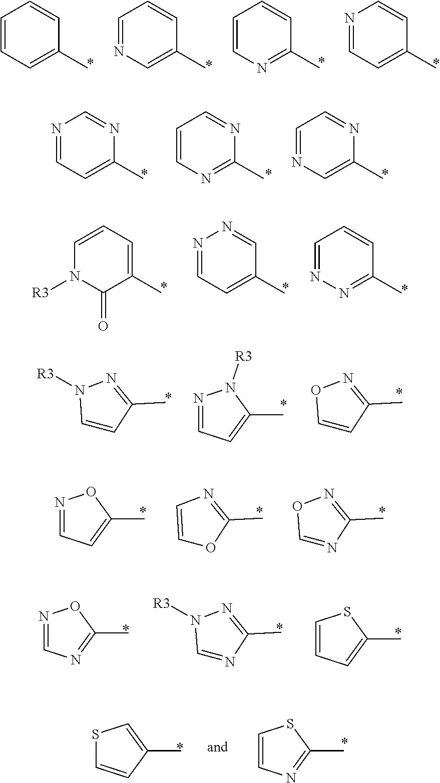
wherein [symbol] indicates a direct bond to NH;
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms;
L is selected from the group consisting of CO and CH₂ or is absent;
L1 is selected from the group consisting of NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇)cycloalkyl-(C₁-C₄)alkyl and deuterated (C₁-C₄)alkyl;
R_{S} is selected from the group consisting of hydrogen and methyl, when L1 is NR;
R_{S} is hydrogen, when L1 is CH₂ or O;
B is a mono- or bi-cyclic heteroaryl ring or a bi-cyclic semisaturated heteroaryl ring;
Y1 and Y2 are substituents of ring B independently selected from the group consisting of hydrogen, (C₁-C₄)alkyl, deuterated (C₁-C₄)alkyl, (C₁-C₄)haloalkyl, (C₁-C₄)alkoxy, (C₁-C₄)haloalkoxy, (C₁-C₄)hydroxyalkyl, halogen, cyano, SF₅, (C₁-C₄)cyanoalkyl, (C₁-C₄)alkyl-sulfonyl, (C₁-C₄)haloalkyl-sulfonyl, CONR1R2, NHCOR1, NR1R2, NR1R2-(C₁-C₄)alkyl, heterocycloalkyl optionally substituted with 1 to 3 halogens, heterocycloalkyl-NH—(C₁-C₄)alkyl, (C₃-C₇)cycloalkyl, (C₁-C₄)alkyl-heterocycloalkyl, (C₁-C₄)alkyl-(C₃-C₇)cycloalkyl, heterocycloalkyl-(C₁-C₄)alkyl, (C₃-C₇)cycloalkyl-(C₁-C₄)alkyl, heterocycloalkyl-(C₁-C₄)alkoxy, (C₃-C₇)cycloalkyl-(C₁-C₄)alkoxy, heterocycloalkyl-oxy, (C₃-C₇)cycloalkyl-oxy, phenyl, (C₁-C₄)alkoxy substituted phenyl, (C₁-C₄)alkyl-thio-(C₁-C₄)alkyl, (C₁-C₄)alkyl-sulfonyl-(C₁-C₄)alkyl, (C₁-C₄)alkoxy-(C₁-C₄)alkyl, (C₁-C₄)haloalkyl-cycloalkyl, (C₁-C₄)alkyl-heterocycloalkyl-carbonyl and monocyclic heteroaryl, optionally substituted with 1 to 3 groups selected from the group consisting of (C₁-C₄)alkyl, (C₁-C₄)alkoxy and (C₁-C₄)alkoxy-(C₁-C₄)alkyl;
R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl;
R3 is selected from the group consisting of (C₁-C₄)alkyl, (C₁-C₄)haloalkyl, (C₁-C₄)alkyl-phenyl and monocyclic heteroaryl;
or a stereoisomer, tautomer, solvate or pharmaceutically acceptable salt thereof;
wherein the compound of formula (I) is not 1-(3,5-dichlorophenyl)-3-(2-(2-methylpyridin-4-yl)-2-azaspiro[3.3]heptan-6-yl)urea.

2. The compound, or stereoisomer tautomer, solvate or pharmaceutically acceptable salt thereof, according to claim 1, wherein, when A ring is phenyl and L1 is NH,
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms.

3. The compound, or stereoisomer, tautomer, solvate or pharmaceutically acceptable salt thereof, according to claim 1, wherein
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms.

4. The compound, or stereoisomer, tautomer, solvate or pharmaceutically acceptable salt thereof, according to claim 1 wherein
A is a ring selected from the group consisting of: 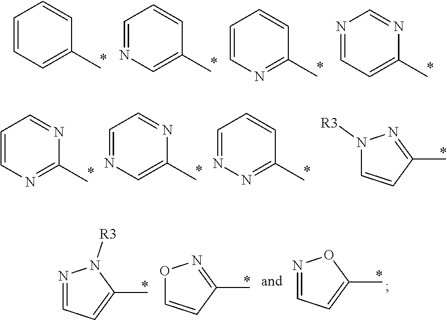
wherein R3 is CH₃ or p-tolyl;
W1, W2 and W3 are selected from the group consisting of hydrogen, CH₃, OCH₃, OCF₃, CF₃, CHF₂, C(CH₃)₃, CH₂CF₃, CF₂CH₃, CF₂CF₃, C(CH₃)₂CF₃, OCF₂H, CH₂OH, cyano, CF₃SO₂, SCF₃, SF₅, F, Br, I, cyclopropyl, morpholino-N-ethoxy, N-acetylpiperidinyl-oxy, N-acetylazetidinyl-oxy, 2,2,2-trifluoro-1-hydroxyethyl, oxetanyloxy and CH₂N(CH₃)₂;
L is selected from the group consisting of CO and CH₂ or is absent;
L1 is selected from the group consisting of NR, CH₂ and O, wherein R is selected from the group consisting of hydrogen, CD₃, cyclopropylmethyl, cyclopropyl, CH₂CF₃, CH₂CH₃ and CH₃;
B is selected from the group consisting of pyridinyl, pyrimidinyl, pyrazinyl, pyridazinyl, imidazolyl, pyrazolyl, pyrazolo[1,5-a]pyrazinyl, pyrazolo[1,5-a]pyridinyl, pyrazolo[3,4-d]pyrimidinyl, pyrazolo[1,5-a]pyrimidinyl, 1H-pyrazolo[3,4-b]pyridinyl, imidazo[1,2-b]pyridazinyl, imidazo[1,2-a]pyrazinyl, 1H-imidazo[1,2-b]pyrazolyl, imidazo[2,1-b]thiazolyl, pyrrolo[2,3-c]pyrimidinyl, pyrrolo[3,2-b]pyridinyl, 1H-pyrrolo[2,3-b]pyridinyl, pyrazolo[5,1-b]thiazolyl, imidazo[1,5-a]pyrazinyl, 5,6-dihydro-8H-imidazo[2,1-c][1,4]oxazinyl, thieno[3,2-d]pyrimidinyl, thieno[3,2-b]pyridinyl, thieno[2,3-d]pyrimidinyl, pyrazolo[5,1-b][1,3]thiazinyl, pyrrolo[3,2-d]pyrimidinyl, pyrrolo[2,3-d]pyrimidinyl and imidazo[1,2-a]pyridinyl;
Y1 is selected from the group consisting of hydrogen, CH₃, OCH₃, OCF₃, CF₃, C(CH₃)₃, C(CH₃)₂CF₃, CONH₂, OCF₂H, CF₂H, CF₂CH₃, CF₂CF₃, CH₂CF₃, CH₂OH, CH₂CH₂OH, CH₂CH₂OCH₃, CH₂CH₂SCH₃, CH₂CH₂F, CH₂SCH₃, SCF₃, SO₂CF₃, CD₃, cyano, NHCOCH₃, NH₂, (C₁-C₄)alkyl-piperazinyl, 4-methylpiperazin-1-yl, piperazinyl, morpholinyl, pyrrolidinyl, hydroxypyrrolidinyl, N-methyl-oxopiperazinyl, S,S-dioxido-thiomorpholinyl, oxetanyl-methyl, oxetanyl, (oxetanylamino)methyl, 1H-pyrazol-4-yl, oxazol-5-yl, pyridin-3-yl, pyrimidin-5-yl, pyridin-4-yl, dimethoxyphenyl, thiazol-5-yl, 3-isoxazol-5-yl and 1-methyl-1H-pyrazol-4-yl, and Y2 is hydrogen; and
R1 and R2 are independently selected from the group consisting of hydrogen, (C₁-C₄)alkyl and optionally substituted heterocycloalkyl, wherein optional substituents are selected from the group consisting of CH₃, OCH₃, OCF₃, CF₃, C(CH₃)₃, C(CH₃)₂CF₃, CONH₂, OCF₂H and CF₂H.

5. The compound, or stereoisomer, tautomer, solvate or pharmaceutically acceptable salt thereof, according to claim 1, wherein L is CO and L1 is NR, wherein the compound is represented by formula (Ia)

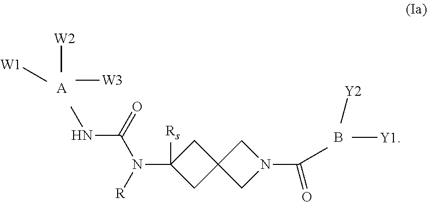

6. The compound, or stereoisomer, tautomer, solvate or pharmaceutically acceptable salt thereof, according to claim 1, wherein L is CH₂ and L1 is NR, wherein the compound is represented by formula (Ib)

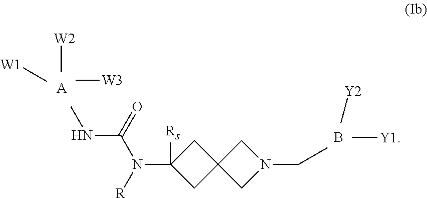

7. The compound, or stereoisomer, tautomer, solvate or pharmaceutically acceptable salt thereof, according to claim 1, wherein L is absent and L1 is NR, wherein the compound is represented by formula (Ic)

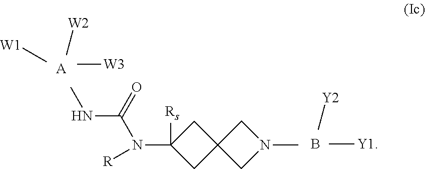

8. The compound, or stereoisomer, tautomer, solvate or pharmaceutically acceptable salt thereof, according to claim 1, wherein L1 is CH₂, wherein the compound is represented by formula (Id) 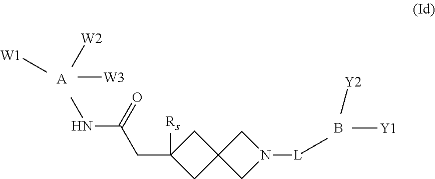
and wherein Rs is hydrogen.

9. The compound, or stereoisomer, tautomer, solvate or pharmaceutically acceptable salt thereof, according to claim 1, wherein L1 is O, wherein the compound is represented by formula (Ie) 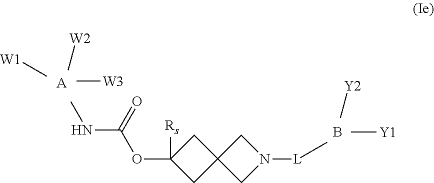
and wherein Rs is hydrogen.

10. The compound, or stereoisomer, tautomer, solvate or pharmaceutically acceptable salt thereof, according to claim 1, wherein the compound is selected from the group consisting of:
1-(2-(imidazo[1,2-a]pyridine-3-carbonyl)-2-azaspiro[3.3]heptan-6-yl)-3-(3-(trifluoromethyl)phenyl)urea;
1-(2-(pyrazolo[1,5-a]pyrazine-3-carbonyl)-2-azaspiro[3.3]heptan-6-yl)-3-(3-(trifluoromethyl)phenyl)urea;
1-(2-(1H-pyrrolo[2,3-b]pyridine-5-carbonyl)-2-azaspiro[3.3]heptan-6-yl)-3-(3-(trifluoromethyl)phenyl)urea;
1-(2-(pyrazolo[5,1-b]thiazole-7-carbonyl)-2-azaspiro[3.3]heptan-6-yl)-3-(3-(trifluoromethyl)phenyl)urea;
1-(2-(6-(4-methylpiperazin-1-yl) imidazo[1,2-a]pyridine-3-carbonyl)-2-azaspiro[3.3]heptan-6-yl)-3-(3-(trifluoromethyl)phenyl)urea;
1-(2-(5,6-dihydro-8H-imidazo[2,1-c][1,4]oxazine-3-carbonyl)-2-azaspiro[3.3]heptan-6-yl)-3-(3-(trifluoromethyl)phenyl)urea;
1-(2-(imidazo[1,2-b]pyridazine-3-carbonyl)-2-azaspiro[3.3]heptan-6-yl)-3-(3-(trifluoromethyl)phenyl)urea;
1-(2-(1-methyl-1H-imidazole-5-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(3-(trifluoromethyl)phenyl)urea;
1-(2-(imidazo[1,2-a]pyrazin-3-ylmethyl)-2-azaspiro[3.3]heptan-6-yl)-3-(3-(trifluoromethyl)phenyl)urea;
1-(2-((1H-pyrazolo[3,4-b]pyridin-5-yl)methyl)-2-azaspiro[3.3]heptan-6-yl)-3-(3-(trifluoromethyl)phenyl)urea;
1-(2-(1H-pyrazolo[3,4-b]pyridin-4-yl)-2-azaspiro[3.3]heptan-6-yl)-3-(3-(trifluoromethyl)phenyl)urea;
1-(3-fluoro-5-(trifluoromethyl)phenyl)-3-(2-(pyrazolo[1,5-a]pyrazine-3-carbonyl)-2-azaspiro[3.3]heptan-6-yl)urea;
1-(2-fluoro-5-(trifluoromethyl)phenyl)-3-(2-(pyrazolo[1,5-a]pyrazine-3-carbonyl)-2-azaspiro[3.3]heptan-6-yl)urea;
1-(5-(tert-butyl) isoxazol-3-yl)-3-(2-(pyrazolo[1,5-a]pyrazine-3-carbonyl)-2-azaspiro[3.3]heptan-6-yl)urea;
1-(3-cyano-5-(trifluoromethyl)phenyl)-3-(2-(pyrazolo[1,5-a]pyrazine-3-carbonyl)-2-azaspiro[3.3]heptan-6-yl)urea;
1-(5-(tert-butyl) isoxazol-3-yl)-3-(2-(pyrazolo[5,1-b]thiazole-7-carbonyl)-2-azaspiro[3.3]heptan-6-yl)urea;
1-(2-(pyrazolo[5,1-b]thiazole-7-carbonyl)-2-azaspiro[3.3]heptan-6-yl)-3-(3-(trifluoromethoxy)phenyl)urea;
1-(2-(pyrazolo[5,1-b]thiazole-7-carbonyl)-2-azaspiro[3.3]heptan-6-yl)-3-(5-(trifluoromethyl)pyridin-3-yl)urea;
1-(2-methoxy-5-(trifluoromethoxy)phenyl)-3-(2-(pyrazolo[5,1-b]thiazole-7-carbonyl)-2-azaspiro[3.3]heptan-6-yl)urea;
1-(4-((dimethylamino)methyl)-3-(trifluoromethyl)phenyl)-3-(2-(pyrazolo[5,1-b]thiazole-7-carbonyl)-2-azaspiro[3.3]heptan-6-yl)urea;
1-methyl-1-(2-(pyrazolo[1,5-a]pyrazine-3-carbonyl)-2-azaspiro[3.3]heptan-6-yl)-3-(3-(trifluoromethyl)phenyl)urea;
1-(2-(imidazo[1,2-a]pyridine-3-carbonyl)-2-azaspiro[3.3]heptan-6-yl)-1-methyl-3-(3-(trifluoromethyl)phenyl)urea;
2-(2-(pyrazolo[5,1-b]thiazole-7-carbonyl)-2-azaspiro[3.3]heptan-6-yl)-N-(3-(trifluoromethyl)phenyl)acetamide;
2-(2-(imidazo[1,2-a]pyridine-3-carbonyl)-2-azaspiro[3.3]heptan-6-yl)-N-(3-(trifluoromethyl)phenyl)acetamide;
2-(2-(pyrazolo[1,5-a]pyrazine-3-carbonyl)-2-azaspiro[3.3]heptan-6-yl)-N-(3-(trifluoromethyl)phenyl)acetamide;
1-(2-(1H-imidazo[1,2-b]pyrazole-7-carbonyl)-2-azaspiro[3.3]heptan-6-yl)-3-(3-(trifluoromethyl)phenyl)urea;
1-(2-(pyrazolo[5,1-b]thiazole-7-carbonyl)-2-azaspiro[3.3]heptan-6-yl)-3-(3-(2,2,2-trifluoroethyl)phenyl)urea;
1-(2-(4-aminopyrazolo[1,5-a]pyridine-3-carbonyl)-2-azaspiro[3.3]heptan-6-yl)-3-(3-(trifluoromethyl)phenyl)urea;
1-(3-methoxyphenyl)-3-(2-(pyrazolo[5,1-b]thiazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(3-(2,2,2-trifluoroethyl)phenyl)urea;
1-(5-isopropylpyridin-3-yl)-3-(2-(pyrazolo[5,1-b]thiazole-7-carbonyl)-2-azaspiro[3.3]heptan-6-yl)urea;
1-(2-methoxy-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(5-(trifluoromethyl)pyridin-3-yl)urea;
1-(5-chloro-2-methoxyphenyl)-3-(2-(1-methyl-1H-imidazo[1,2-b]pyrazole-7-carbonyl)-2-azaspiro[3.3]heptan-6-yl)urea;
1-(3-cyano-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-(pyrazolo[1,5-a]pyridine-3-carbonyl)-2-azaspiro[3.3]heptan-6-yl)-3-(3-(trifluoromethyl)phenyl)urea;
1-(3-(hydroxymethyl)-5-(trifluoromethyl)phenyl)-3-(2-(pyrazolo[5,1-b]thiazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(3-(pentafluoro-16-sulfaneyl)phenyl)urea;
1-(3-(tert-butyl)-1-(p-tolyl)-1H-pyrazol-5-yl)-3-(2-(1-methyl-1H-imidazo[1,2-b]pyrazole-7-carbonyl)-2-azaspiro[3.3]heptan-6-yl)urea;
1-(2-isonicotinoyl-2-azaspiro[3.3]heptan-6-yl)-3-(3-(trifluoromethyl)phenyl)urea;
1-(2-chloro-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-(1H-pyrrolo[3,2-b]pyridine-7-carbonyl)-2-azaspiro[3.3]heptan-6-yl)-3-(3-(trifluoromethyl)phenyl)urea;
1-methyl-1-(2-(pyrazolo[5,1-b]thiazole-7-carbonyl)-2-azaspiro[3.3]heptan-6-yl)-3-(5-(trifluoromethyl)pyridin-3-yl)urea;
1-(2-methoxy-5-(trifluoromethoxy)phenyl)-3-(2-(thieno[3,2-b]pyridine-7-carbonyl)-2-azaspiro[3.3]heptan-6-yl)urea;
1-(2-(thieno[3,2-b]pyridine-7-carbonyl)-2-azaspiro[3.3]heptan-6-yl)-3-(5-(trifluoromethyl)pyridin-3-yl)urea;
azaspiro[3.3]heptan-6-yl)-3-(3-(2,2,2-trifluoroethyl)phenyl)urea;
3-(5-(trifluoromethoxy)pyridin-3-yl)urea;
3-(2-methoxy-5-(trifluoromethoxy)phenyl)-1-methyl-1-(2-(1-methyl-1H-imidazo[1,2-b]pyrazole-7-carbonyl)-2-azaspiro[3.3]heptan-6-yl)urea;
3-(3-cyano-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(imidazo[1,2-b]pyridazine-3-carbonyl)-2-azaspiro[3.3]heptan-6-yl)-3-(2-methoxy-5-(trifluoromethoxy)phenyl)urea;
1-(2-methoxy-5-(trifluoromethoxy)phenyl)-3-(2-(pyrazolo[1,5-a]pyrimidine-3-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(2-(2-morpholinoethoxy)-5-(trifluoromethyl)phenyl)urea;
1-(2-(imidazo[1,2-a]pyrazine-3-carbonyl)-2-azaspiro[3.3]heptan-6-yl)-3-(2-methoxy-5-(trifluoromethoxy)phenyl)urea;
1-(1-methyl-2-oxo-5-(trifluoromethyl)-1,2-dihydropyridin-3-yl)-3-(2-(pyrazolo[5,1-b]thiazole-7-carbonyl)-2-azaspiro[3.3]heptan-6-yl)urea;
1-(2-(pyrazolo[5,1-b]thiazole-7-carbonyl)-2-azaspiro[3.3]heptan-6-yl)-3-(4-(trifluoromethyl)pyridin-2-yl)urea;
3 3-(3-fluoro-5-(trifluoro methyl)phenyl)-1-methyl-1-(2-(pyrazolo[5,1-b]thiazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(2-methyl-5-(trifluoromethoxy)phenyl)urea;
1-(2-methoxy-5-(trifluoromethoxy)phenyl)-3-(2-(thieno[3,2-d]pyrimidine-4-carbonyl)-2-azaspiro[3.3]heptan-6-yl)urea;
1-(3-(difluoromethyl)phenyl)-3-(2-(1-methyl-1H-imidazo[1,2-b]pyrazole-7-carbonyl)-2-azaspiro[3.3]heptan-6-yl)urea;
3-(3-fluoro-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-methoxy-5-(trifluoromethyl)phenyl)-3-(2-(1-methyl-1H-imidazo[1,2-b]pyrazole-7-carbonyl)-2-azaspiro[3.3]heptan-6-yl)urea;
1-(4-(difluoromethyl)pyridin-2-yl)-3-(2-(1-methyl-1H-imidazo[1,2-b]pyrazole-7-carbonyl)-2-azaspiro[3.3]heptan-6-yl)urea;
1-(3-(difluoromethoxy)phenyl)-3-(2-(1-methyl-1H-imidazo[1,2-b]pyrazole-7-carbonyl)-2-azaspiro[3.3]heptan-6-yl)urea;
1-(2-methoxy-5-(trifluoromethoxy)phenyl)-3-(2-(pyrazolo[1,5-a]pyridine-3-carbonyl)-2-azaspiro[3.3]heptan-6-yl)urea;
1-(2-methoxy-5-(trifluoromethoxy)phenyl)-3-(2-(5-(4-methylpiperazin-1-yl) pyrazolo[1,5-a]pyridine-3-carbonyl)-2-azaspiro[3.3]heptan-6-yl)urea;
1-(2-(1-ethyl-1H-imidazo[1,2-b]pyrazole-7-carbonyl)-2-azaspiro[3.3]heptan-6-yl)-3-(3-(trifluoromethyl)phenyl)urea;
1-(3-cyano-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-fluoro-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(4-(trifluoromethoxy)pyridin-2-yl)urea;
1-(4-(difluoromethyl)pyridin-2-yl)-3-(2-(thieno[3,2-d]pyrimidine-4-carbonyl)-2-azaspiro[3.3]heptan-6-yl)urea;
3-(4-(trifluoromethyl)pyridin-2-yl)urea;
1-(2-(thieno[3,2-d]pyrimidine-4-carbonyl)-2-azaspiro[3.3]heptan-6-yl)-3-(5-(trifluoromethyl)pyridin-3-yl)urea;
3-(2-methoxy-5-(trifluoromethoxy)phenyl)-1-methyl-1-(2-(thieno[3,2-d]pyrimidine-4-carbonyl)-2-azaspiro[3.3]heptan-6-yl)urea;
1-(3-(hydroxymethyl)-2-methoxy-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-(thieno[3,2-d]pyrimidine-4-carbonyl)-2-azaspiro[3.3]heptan-6-yl)-3-(3-(trifluoromethyl)phenyl)urea;
1-(3-(tert-butyl)-1-(p-tolyl)-1H-pyrazol-5-yl)-3-(2-(pyrazolo[5,1-b]thiazole-7-carbonyl)-2-azaspiro[3.3]heptan-6-yl)urea;
3-(2-methoxy-5-(trifluoromethoxy)phenyl)-1-methyl-1-(2-(5-(4-methylpiperazin-1-yl) pyrazolo[1,5-a]pyridine-3-carbonyl)-2-azaspiro[3.3]heptan-6-yl)urea;
1-(3-(tert-butyl)-1-(p-tolyl)-1H-pyrazol-5-yl)-3-(2-(thieno[3,2-d]pyrimidine-4-carbonyl)-2-azaspiro[3.3]heptan-6-yl)urea;
1-(5-chloro-2-methoxypyridin-3-yl)-3-(2-(1-methyl-1H-imidazo[1,2-b]pyrazole-7-carbonyl)-2-azaspiro[3.3]heptan-6-yl)urea;
1-(2-(5-(4-methylpiperazin-1-yl) pyrazolo[1,5-a]pyridine-3-carbonyl)-2-azaspiro[3.3]heptan-6-yl)-3-(3-(trifluoromethyl)phenyl)urea;
1-ethyl-1-(2-(1-methyl-1H-imidazo[1,2-b]pyrazole-7-carbonyl)-2-azaspiro[3.3]heptan-6-yl)-3-(3-(trifluoromethyl)phenyl)urea;
1-ethyl-3-(3-fluoro-5-(trifluoromethyl)phenyl)-1-(2-(1-methyl-1H-imidazo[1,2-b]pyrazole-7-carbonyl)-2-azaspiro[3.3]heptan-6-yl)urea;
1-(3-cyano-5-(trifluoromethyl)phenyl)-3-(2-(5-(4-methylpiperazin-1-yl) pyrazolo[1,5-a]pyridine-3-carbonyl)-2-azaspiro[3.3]heptan-6-yl)urea;
1-(2-(thieno[2,3-d]pyrimidine-4-carbonyl)-2-azaspiro[3.3]heptan-6-yl)-3-(3-(trifluoromethyl)phenyl)urea;
1-(3-(difluoromethoxy)phenyl)-3-(2-(thieno[3,2-d]pyrimidine-4-carbonyl)-2-azaspiro[3.3]heptan-6-yl)urea;
1-(2-(thieno[3,2-d]pyrimidine-4-carbonyl)-2-azaspiro[3.3]heptan-6-yl)-3-(4-(trifluoromethyl)pyridin-2-yl)urea;
1-(5-(difluoromethoxy)-2-methoxyphenyl)-3-(2-(1-methyl-1H-imidazo[1,2-b]pyrazole-7-carbonyl)-2-azaspiro[3.3]heptan-6-yl)urea;
1-(2-chloro-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-3-(3-(trifluoromethyl)phenyl)urea;
3-(3-(hydroxymethyl)-5-(trifluoromethyl)phenyl)-1-methyl-1-(2-(pyrazolo[5,1-b]thiazole-7-carbonyl)-2-azaspiro[3.3]heptan-6-yl)urea;
1-(3-(tert-butyl)-1-methyl-1H-pyrazol-5-yl)-3-(2-(thieno[3,2-d]pyrimidine-4-carbonyl)-2-azaspiro[3.3]heptan-6-yl)urea;
1-(2-(imidazo[2,1-b]thiazole-5-carbonyl)-2-azaspiro[3.3]heptan-6-yl)-3-(3-(trifluoromethyl)phenyl)urea;
1-(2-(pyrazolo[5,1-b]thiazole-7-carbonyl)-2-azaspiro[3.3]heptan-6-yl)-3-(4-(trifluoromethoxy)pyridin-2-yl)urea;
azaspiro[3.3]heptan-6-yl)-3-(3-(pentafluoro-16-sulfaneyl)phenyl)urea;
1-(2-(6-methylthieno[3,2-d]pyrimidine-4-carbonyl)-2-azaspiro[3.3]heptan-6-yl)-3-(3-(trifluoromethyl)phenyl)urea;
1-(5-(tert-butyl)-1-methyl-1H-pyrazol-3-yl)-3-(2-(thieno[3,2-d]pyrimidine-4-carbonyl)-2-azaspiro[3.3]heptan-6-yl)urea;
1-(2-(thieno[3,2-d]pyrimidine-4-carbonyl)-2-azaspiro[3.3]heptan-6-yl)-3-(4-(trifluoromethoxy)pyridin-2-yl)urea;
1-(2-(1-(2-hydroxyethyl)-1H-imidazo[1,2-b]pyrazole-7-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(6-(trifluoromethyl)pyrimidin-4-yl)urea;
1-methyl-1-(2-(1-methyl-1H-imidazo[1,2-b]pyrazole-7-carbonyl)-2-azaspiro[3.3]heptan-6-yl)-3-(2-methyl-5-(trifluoromethoxy)phenyl)urea;
1-methyl-1-(2-(1-methyl-1H-imidazo[1,2-b]pyrazole-7-carbonyl)-2-azaspiro[3.3]heptan-6-yl)-3-(4-(trifluoromethoxy)pyridin-2-yl)urea;
1-(3-fluoro-5-(trifluoromethyl)phenyl)-3-(2-(1-methyl-1H-imidazo[1,2-b]pyrazole-7-carbonyl)-2-azaspiro[3.3]heptan-6-yl)urea;
1-(2-methoxy-5-(trifluoromethyl)pyridin-3-yl)-3-(2-(1-methyl-1H-imidazo[1,2-b]pyrazole-7-carbonyl)-2-azaspiro[3.3]heptan-6-yl)urea;
1-(4-chloro-3-(trifluoromethyl)phenyl)-3-(2-(1-methyl-1H-imidazo[1,2-b]pyrazole-7-carbonyl)-2-azaspiro[3.3]heptan-6-yl)urea;
1-(2-methoxy-5-(pentafluoro-16-sulfaneyl)phenyl)-3-(2-(1-methyl-1H-imidazo[1,2-b]pyrazole-7-carbonyl)-2-azaspiro[3.3]heptan-6-yl)urea;
1-(3-(hydroxymethyl)-5-(trifluoromethyl)phenyl)-3-(2-(thieno[3,2-d]pyrimidine-4-carbonyl)-2-azaspiro[3.3]heptan-6-yl)urea;
3-(3-cyano-5-(trifluoromethoxy)phenyl)-1-methyl-1-(2-(1-methyl-1H-imidazo[1,2-b]pyrazole-7-carbonyl)-2-azaspiro[3.3]heptan-6-yl)urea;
1-(4-((dimethylamino)methyl)-3-(trifluoromethyl)phenyl)-3-(2-(thieno[3,2-d]pyrimidine-4-carbonyl)-2-azaspiro[3.3]heptan-6-yl)urea;
1-(2-(6-(hydroxymethyl) pyrazolo[1,5-a]pyridine-3-carbonyl)-2-azaspiro[3.3]heptan-6-yl)-3-(3-(trifluoromethyl)phenyl)urea;
1-methyl-1-(2-(pyrazolo[5,1-b]thiazole-7-carbonyl)-2-azaspiro[3.3]heptan-6-yl)-3-(4-(trifluoromethoxy)pyridin-2-yl)urea;
1-(2-(1H-pyrrolo[2,3-b]pyridine-4-carbonyl)-2-azaspiro[3.3]heptan-6-yl)-3-(3-(trifluoromethyl)phenyl)urea;
1-(2-(6-((oxetan-3-ylamino)methyl) pyrazolo[1,5-a]pyridine-3-carbonyl)-2-azaspiro[3.3]heptan-6-yl)-3-(3-(trifluoromethyl)phenyl)urea;
1-methyl-1-(2-(1-methyl-1H-imidazo[1,2-b]pyrazole-7-carbonyl)-2-azaspiro[3.3]heptan-6-yl)-3-(3-(perfluoroethyl)phenyl)urea;
1-(4-fluoro-3-(trifluoromethoxy)phenyl)-3-(2-(1-methyl-1H-imidazo[1,2-b]pyrazole-7-carbonyl)-2-azaspiro[3.3]heptan-6-yl)urea;
1-(3-cyano-5-(pentafluoro-16-sulfaneyl)phenyl)-3-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-3-(3-(pentafluoro-16-sulfaneyl)phenyl)urea;
1-(4-fluoro-3-(trifluoromethyl)phenyl)-3-(2-(1-methyl-1H-imidazo[1,2-b]pyrazole-7-carbonyl)-2-azaspiro[3.3]heptan-6-yl)urea;
1-(2-(1H-pyrazolo[3,4-d]pyrimidine-4-carbonyl)-2-azaspiro[3.3]heptan-6-yl)-3-(3-(trifluoromethyl)phenyl)urea;
1-(2-fluoro-5-(trifluoromethyl)phenyl)-3-(2-(1-methyl-1H-imidazo[1,2-b]pyrazole-7-carbonyl)-2-azaspiro[3.3]heptan-6-yl)urea;
1-(3-fluoro-5-(trifluoromethoxy)phenyl)-3-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-3-(3-((trifluoromethyl)thio)phenyl)urea;
1-(2-(1-methyl-1H-imidazo[1,2-b]pyrazole-7-carbonyl)-2-azaspiro[3.3]heptan-6-yl)-3-(2-(oxetan-3-yloxy)-5-(trifluoromethyl)phenyl)urea;
1-(2-(1-methyl-1H-imidazo[1,2-b]pyrazole-7-carbonyl)-2-azaspiro[3.3]heptan-6-yl)-3-(3-(perfluoroethyl)phenyl)urea;
1-(2-(1H-pyrazolo[3,4-b]pyridine-4-carbonyl)-2-azaspiro[3.3]heptan-6-yl)-1-methyl-3-(3-(trifluoromethyl)phenyl)urea;
1-methyl-1-(2-(1-methyl-1H-imidazo[1,2-b]pyrazole-7-carbonyl)-2-azaspiro[3.3]heptan-6-yl)-3-(4-(trifluoromethyl)pyridin-2-yl)urea;
1-methyl-1-(2-(thieno[3,2-d]pyrimidine-4-carbonyl)-2-azaspiro[3.3]heptan-6-yl)-3-(5-(trifluoromethyl)pyridin-3-yl)urea;
3-(4-fluoro-3-(trifluoromethyl)phenyl)-1-methyl-1-(2-(1-methyl-1H-imidazo[1,2-b]pyrazole-7-carbonyl)-2-azaspiro[3.3]heptan-6-yl)urea;
3-(2-fluoro-5-(trifluoromethoxy)phenyl)-1-methyl-1-(2-(1-methyl-1H-imidazo[1,2-b]pyrazole-7-carbonyl)-2-azaspiro[3.3]heptan-6-yl)urea;
1-methyl-1-(2-(pyrazolo[5,1-b]thiazole-7-carbonyl)-2-azaspiro[3.3]heptan-6-yl)-3-(3-(trifluoromethyl)phenyl)urea;
azaspiro[3.3]heptan-6-yl)-3-(3-(trifluoromethyl)phenyl)urea;
1-(2-(2,3-dihydropyrazolo[5,1-b]thiazole-7-carbonyl)-2-azaspiro[3.3]heptan-6-yl)-3-(3-(trifluoromethyl)phenyl)urea;
1-(2-(6,7-dihydro-5H-pyrazolo[5,1-b][1,3]thiazine-3-carbonyl)-2-azaspiro[3.3]heptan-6-yl)-3-(3-(trifluoromethyl)phenyl)urea;
1-(2-(thieno[3,2-b]pyridine-7-carbonyl)-2-azaspiro[3.3]heptan-6-yl)-3-(3-(trifluoromethyl)phenyl)urea;
1-(2-(1H-pyrazolo[3,4-b]pyridin-5-yl)-2-azaspiro[3.3]heptan-6-yl)-3-(3-(trifluoromethyl)phenyl)urea;
N-(2-methoxy-5-(trifluoromethoxy)phenyl)-2-(2-(thieno[3,2-b]pyridine-7-carbonyl)-2-azaspiro[3.3]heptan-6-yl) acetamide;
2-(2-(thieno[3,2-b]pyridine-7-carbonyl)-2-azaspiro[3.3]heptan-6-yl)-N-(3-(trifluoromethyl)phenyl)acetamide;
2-(2-(1-methyl-1H-imidazo[1,2-b]pyrazole-7-carbonyl)-2-azaspiro[3.3]heptan-6-yl)-N-(3-(trifluoromethyl)phenyl)acetamide;
N-(2-methoxy-5-(trifluoromethoxy)phenyl)-2-(2-(1-methyl-1H-imidazo[1,2-b]pyrazole-7-carbonyl)-2-azaspiro[3.3]heptan-6-yl) acetamide;
2-(1-methyl-1H-imidazo[1,2-b]pyrazole-7-carbonyl)-2-azaspiro[3.3]heptan-6-yl(3-(trifluoromethyl)phenyl)carbamate;
2-(1-methyl-1H-imidazo[1,2-b]pyrazole-7-carbonyl)-2-azaspiro[3.3]heptan-6-yl(2-methoxy-5-(trifluoromethoxy)phenyl)carbamate;
1-(2-(1H-pyrazolo[3,4-b]pyridine-4-carbonyl)-2-azaspiro[3.3]heptan-6-yl)-3-(4-(trifluoromethyl)pyridin-2-yl)urea;
2-(1-methyl-1H-imidazo[1,2-b]pyrazole-7-carbonyl)-2-azaspiro[3.3]heptan-6-yl(5-(trifluoromethyl)pyridin-3-yl)carbamate;
2-(pyrazolo[5,1-b]thiazole-7-carbonyl)-2-azaspiro[3.3]heptan-6-yl(3-(trifluoromethyl)phenyl)carbamate;
1-(2-(7H-pyrrolo[2,3-d]pyrimidine-4-carbonyl)-2-azaspiro[3.3]heptan-6-yl)-3-(3-(trifluoromethyl)phenyl)urea;
1-(2-(imidazo[1,5-a]pyrazin-8-yl)-2-azaspiro[3.3]heptan-6-yl)-3-(3-(trifluoromethyl)phenyl)urea;
1-methyl-1-(2-(1-methyl-1H-imidazo[1,2-b]pyrazole-7-carbonyl)-2-azaspiro[3.3]heptan-6-yl)-3-(4-(2,2,2-trifluoroethyl)pyridin-2-yl)urea;
1-methyl-3-(2-methyl-5-(trifluoromethoxy)phenyl)-1-(2-(pyrazolo[5,1-b]thiazole-7-carbonyl)-2-azaspiro[3.3]heptan-6-yl)urea;
1-(2-methoxy-5-(2,2,2-trifluoroethyl)phenyl)-3-(2-(1-methyl-1H-imidazo[1,2-b]pyrazole-7-carbonyl)-2-azaspiro[3.3]heptan-6-yl)urea;
1-(2-(1H-pyrazolo[3,4-b]pyridine-4-carbonyl)-2-azaspiro[3.3]heptan-6-yl)-3-(3-(2,2,2-trifluoroethyl)phenyl)urea;
1-(2-(1H-pyrazolo[3,4-b]pyridine-4-carbonyl)-2-azaspiro[3.3]heptan-6-yl)-3-(4-(trifluoromethoxy)pyridin-2-yl)urea;
1-(4-cyclopropylpyridin-2-yl)-3-(2-(1-methyl-1H-imidazo[1,2-b]pyrazole-7-carbonyl)-2-azaspiro[3.3]heptan-6-yl)urea;
3-(4-cyclopropylpyridin-2-yl)-1-methyl-1-(2-(1-methyl-1H-imidazo[1,2-b]pyrazole-7-carbonyl)-2-azaspiro[3.3]heptan-6-yl)urea;
3-(5-cyclopropylpyridin-3-yl)-1-methyl-1-(2-(1-methyl-1H-imidazo[1,2-b]pyrazole-7-carbonyl)-2-azaspiro[3.3]heptan-6-yl)urea;
1-(methyl-d3)-1-(2-(1-methyl-1H-imidazo[1,2-b]pyrazole-7-carbonyl)-2-azaspiro[3.3]heptan-6-yl)-3-(3-(trifluoromethyl)phenyl)urea);
1-(2-(1H-pyrazolo[3,4-b]pyridine-4-carbonyl)-2-azaspiro[3.3]heptan-6-yl)-3-(5-(trifluoromethyl)pyridin-3-yl)urea;
1-(2-(5-methyl-5H-pyrrolo[3,2-d]pyrimidine-4-carbonyl)-2-azaspiro[3.3]heptan-6-yl)-3-(3-(trifluoromethyl)phenyl)urea;
1-(2-(1-methyl-1H-imidazo[1,2-b]pyrazole-7-carbonyl)-2-azaspiro[3.3]heptan-6-yl)-3-(3-((trifluoromethyl) sulfonyl)phenyl)urea;
1-(2-(1H-pyrazolo[3,4-b]pyridine-4-carbonyl)-2-azaspiro[3.3]heptan-6-yl)-3-(3-((trifluoromethyl)thio)phenyl)urea;
1-(2-(1H-pyrazolo[3,4-b]pyridin-5-yl)-2-azaspiro[3.3]heptan-6-yl)-3-(5-(trifluoromethyl)pyridin-2-yl)urea;
3-(4-(difluoromethyl)pyridin-2-yl)-1-methyl-1-(2-(1-methyl-1H-imidazo[1,2-b]pyrazole-7-carbonyl)-2-azaspiro[3.3]heptan-6-yl)urea;
1-(2-methyl-5-(trifluoromethyl)pyridin-3-yl)-3-(2-(pyrazolo[5,1-b]thiazole-7-carbonyl)-2-azaspiro[3.3]heptan-6-yl)urea;
1-(2-(1-(oxetan-3-ylmethyl)-1H-imidazo[1,2-b]pyrazole-7-carbonyl)-2-azaspiro[3.3]heptan-6-yl)-3-(3-(trifluoromethyl)phenyl)urea;
1-(2-(1-(oxetan-3-yl)-1H-imidazo[1,2-b]pyrazole-7-carbonyl)-2-azaspiro[3.3]heptan-6-yl)-3-(3-(trifluoromethyl)phenyl)urea;
1-(6-cyano-4-(trifluoromethyl)pyridin-2-yl)-3-(2-(1-methyl-1H-imidazo[1,2-b]pyrazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(5-(trifluoromethyl)pyridin-3-yl)urea;
azaspiro[3.3]heptan-6-yl)-3-(6-(trifluoromethyl)pyrimidin-4-yl)urea;
3-(2-methyl-5-(trifluoromethyl)pyridin-3-yl)urea;
1-methyl-1-(2-(1-methyl-1H-imidazo[1,2-b]pyrazole-7-carbonyl)-2-azaspiro[3.3]heptan-6-yl)-3-(5-(trifluoromethoxy)pyridin-3-yl)urea;
3-(5-(1,1-difluoroethyl)pyridin-3-yl)-1-methyl-1-(2-(1-methyl-1H-imidazo[1,2-b]pyrazole-7-carbonyl)-2-azaspiro[3.3]heptan-6-yl)urea;
1-(2-(1-methyl-1H-imidazo[1,2-b]pyrazole-7-carbonyl)-2-azaspiro[3.3]heptan-6-yl)-1-(2,2,2-trifluoroethyl)-3-(3-(trifluoromethyl)phenyl)urea;
1-methyl-1-(2-(pyrazolo[1,5-a]pyrazine-3-carbonyl)-2-azaspiro[3.3]heptan-6-yl)-3-(5-(trifluoromethyl)pyridin-3-yl)urea;
1-methyl-1-(2-(pyrazolo[1,5-a]pyrazine-3-carbonyl)-2-azaspiro[3.3]heptan-6-yl)-3-(5-(trifluoromethoxy)pyridin-3-yl)urea;
3-(4-(difluoromethoxy)pyridin-2-yl)-1-methyl-1-(2-(1-methyl-1H-imidazo[1,2-b]pyrazole-7-carbonyl)-2-azaspiro[3.3]heptan-6-yl)urea;
1-methyl-1-(2-(pyrazolo[5,1-b]thiazole-7-carbonyl)-2-azaspiro[3.3]heptan-6-yl)-3-(6-(trifluoromethyl)pyrimidin-4-yl)urea;
1-methyl-1-(2-(pyrazolo[5,1-b]thiazole-7-carbonyl)-2-azaspiro[3.3]heptan-6-yl)-3-(5-(trifluoromethoxy)pyridin-3-yl)urea;
1-methyl-1-(2-(1-methyl-1H-imidazo[1,2-b]pyrazole-7-carbonyl)-2-azaspiro[3.3]heptan-6-yl)-3-(6-methyl-5-(trifluoromethyl)pyridin-3-yl)urea;
1-(2-(1-methyl-1H-imidazo[1,2-b]pyrazole-7-carbonyl)-2-azaspiro[3.3]heptan-6-yl)-3-(6-methyl-5-(trifluoromethyl)pyridin-3-yl)urea;
1-(2-(6-(1-methyl-1H-pyrazol-4-yl) pyrazolo[1,5-a]pyridine-3-carbonyl)-2-azaspiro[3.3]heptan-6-yl)-3-(3-(trifluoromethyl)phenyl)urea);
1-(2-(5-(1,1-dioxidothiomorpholino) pyrazolo[1,5-a]pyridine-3-carbonyl)-2-azaspiro[3.3]heptan-6-yl)-3-(3-(trifluoromethyl)phenyl)urea;
1-(2-(5-aminopyrazolo[1,5-a]pyridine-3-carbonyl)-2-azaspiro[3.3]heptan-6-yl)-3-(3-(trifluoromethyl)phenyl)urea;
1-(2-(5-(3-hydroxypyrrolidin-1-yl) pyrazolo[1,5-a]pyridine-3-carbonyl)-2-azaspiro[3.3]heptan-6-yl)-3-(3-(trifluoromethyl)phenyl)urea;
1-(2-(5-(4-methyl-2-oxopiperazin-1-yl) pyrazolo[1,5-a]pyridine-3-carbonyl)-2-azaspiro[3.3]heptan-6-yl)-3-(3-(trifluoromethyl)phenyl)urea;
1-cyclopropyl-1-(2-(1-methyl-1H-imidazo[1,2-b]pyrazole-7-carbonyl)-2-azaspiro[3.3]heptan-6-yl)-3-(3-(trifluoromethyl)phenyl)urea;
1-(2-(1H-pyrazolo[3,4-b]pyridine-4-carbonyl)-2-azaspiro[3.3]heptan-6-yl)-1-methyl-3-(2-methyl-5-(trifluoromethoxy)phenyl)urea;
1-(2-(1H-pyrazolo[3,4-b]pyridine-4-carbonyl)-2-azaspiro[3.3]heptan-6-yl)-1-methyl-3-(4-(trifluoromethoxy)pyridin-2-yl)urea;
1-methyl-1-(2-(pyrazolo[1,5-a]pyrazine-3-carbonyl)-2-azaspiro[3.3]heptan-6-yl)-3-(4-(trifluoromethoxy)pyridin-2-yl)urea;
1-(6-methyl-2-(thieno[3,2-d]pyrimidine-4-carbonyl)-2-azaspiro[3.3]heptan-6-yl)-3-(3-(trifluoromethyl)phenyl)urea;
1-(2-(1-methyl-3-morpholino-1H-imidazo[1,2-b]pyrazole-7-carbonyl)-2-azaspiro[3.3]heptan-6-yl)-3-(3-(trifluoromethyl)phenyl)urea;
3-(2-fluoro-3-methyl-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(1-(2-(methylthio)ethyl)-1H-imidazo[1,2-b]pyrazole-7-carbonyl)-2-azaspiro[3.3]heptan-6-yl)-3-(3-(trifluoromethyl)phenyl)urea;
1-(2-(1-(2-fluoroethyl)-1H-imidazo[1,2-b]pyrazole-7-carbonyl)-2-azaspiro[3.3]heptan-6-yl)-1-methyl-3-(3-(trifluoromethyl)phenyl)urea;
1-(2-(1-((methylthio)methyl)-1H-imidazo[1,2-b]pyrazole-7-carbonyl)-2-azaspiro[3.3]heptan-6-yl)-3-(3-(trifluoromethyl)phenyl)urea;
3-(5-bromopyridin-3-yl)-1-methyl-1-(2-(1-methyl-1H-imidazo[1,2-b]pyrazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(3-(1-(trifluoromethyl)cyclopropyl)phenyl)urea;
1-methyl-1-(2-(1-methyl-1H-imidazo[1,2-b]pyrazole-7-carbonyl)-2-azaspiro[3.3]heptan-6-yl)-3-(5-(1,1,1-trifluoro-2-methylpropan-2-yl) isoxazol-3-yl)urea;
3-(4-bromopyridin-2-yl)-1-methyl-1-(2-(1-methyl-1H-imidazo[1,2-b]pyrazole-7-carbonyl)-2-azaspiro[3.3]heptan-6-yl)urea;
1-(2-(7H-pyrrolo[2,3-d]pyrimidine-4-carbonyl)-2-azaspiro[3.3]heptan-6-yl)-1-methyl-3-(2-methyl-5-(trifluoromethoxy)phenyl)urea;
1-(2-(1-(2-methoxyethyl)-1H-imidazo[1,2-b]pyrazole-7-carbonyl)-2-azaspiro[3.3]heptan-6-yl)-1-methyl-3-(4-(trifluoromethoxy)pyridin-2-yl)urea;
1-(2-(1-(2-methoxyethyl)-1H-imidazo[1,2-b]pyrazole-7-carbonyl)-2-azaspiro[3.3]heptan-6-yl)-1-methyl-3-(3-(trifluoromethyl)phenyl)urea;
1-(2-(7H-pyrrolo[2,3-d]pyrimidine-4-carbonyl)-2-azaspiro[3.3]heptan-6-yl)-1-methyl-3-(5-(trifluoromethoxy)pyridin-3-yl)urea;
1-(2-(7H-pyrrolo[2,3-d]pyrimidine-4-carbonyl)-2-azaspiro[3.3]heptan-6-yl)-1-methyl-3-(4-(trifluoromethoxy)pyridin-2-yl)urea;
1-(2-(7H-pyrrolo[2,3-d]pyrimidine-4-carbonyl)-2-azaspiro[3.3]heptan-6-yl)-1-methyl-3-(5-(trifluoromethyl)pyridin-3-yl)urea;
1-methyl-1-(2-(1-methyl-1H-imidazo[1,2-b]pyrazole-7-carbonyl)-2-azaspiro[3.3]heptan-6-yl)-3-(5-((trifluoromethyl)thio)pyridin-3-yl)urea;
1-methyl-1-(2-(1-methyl-1H-imidazo[1,2-b]pyrazole-7-carbonyl)-2-azaspiro[3.3]heptan-6-yl)-3-(5-(trifluoromethyl)pyridazin-3-yl)urea;
1-(2-(1-(2-hydroxyethyl)-1H-imidazo[1,2-b]pyrazole-7-carbonyl)-2-azaspiro[3.3]heptan-6-yl)-1-methyl-3-(4-(trifluoromethyl)pyridin-2-yl)urea;
1-(2-(1-(2-hydroxyethyl)-1H-imidazo[1,2-b]pyrazole-7-carbonyl)-2-azaspiro[3.3]heptan-6-yl)-1-methyl-3-(3-(pentafluoro-16-sulfaneyl)phenyl)urea;
1-(2-(1-(2-hydroxyethyl)-1H-imidazo[1,2-b]pyrazole-7-carbonyl)-2-azaspiro[3.3]heptan-6-yl)-1-methyl-3-(4-(trifluoromethoxy)pyridin-2-yl)urea;
1-(3-(hydroxymethyl)-2-methoxy-5-(trifluoromethoxy)phenyl)-3-(2-(pyrazolo[1,5-a]pyrazine-3-carbonyl)-2-azaspiro[3.3]heptan-6-yl)urea;
1-(2-(1-(2-methoxyethyl)-1H-imidazo[1,2-b]pyrazole-7-carbonyl)-2-azaspiro[3.3]heptan-6-yl)-1-methyl-3-(5-(trifluoromethyl)pyridin-3-yl)urea;
1-methyl-1-(2-(5-(pyrrolidin-1-yl) pyrazolo[1,5-a]pyridine-3-carbonyl)-2-azaspiro[3.3]heptan-6-yl)-3-(5-(trifluoromethyl)pyridin-3-yl)urea;
3-(3-(tert-butyl) isoxazol-5-yl)-1-methyl-1-(2-(1-methyl-1H-imidazo[1,2-b]pyrazole-7-carbonyl)-2-azaspiro[3.3]heptan-6-yl)urea;
1-methyl-1-(2-(pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(pyrazolo[5,1-b]thiazole-7-carbonyl)-2-azaspiro[3.3]heptan-6-yl)-3-(4-(trifluoromethyl)pyridin-2-yl)urea;
1-(2-(1H-pyrazolo[3,4-b]pyridin-5-yl)-2-azaspiro[3.3]heptan-6-yl)-3-(3-(hydroxymethyl)-2-methoxy-5-(trifluoromethoxy)phenyl)-1-methylurea;
1-(2-(1H-pyrazolo[3,4-b]pyridin-5-yl)-2-azaspiro[3.3]heptan-6-yl)-1-methyl-3-(4-(trifluoromethoxy)pyridin-2-yl)urea;
1-(2-(1H-pyrrolo[2,3-b]pyridin-5-yl)-2-azaspiro[3.3]heptan-6-yl)-1-methyl-3-(5-(trifluoromethyl)pyridin-3-yl)urea;
1-(2-(1H-pyrazolo[3,4-b]pyridin-5-yl)-2-azaspiro[3.3]heptan-6-yl)-1-methyl-3-(5-(trifluoromethoxy)pyridin-3-yl)urea;
3-(5-(tert-butyl) isoxazol-3-yl)-1-methyl-1-(2-(1-methyl-1H-imidazo[1,2-b]pyrazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(3-methyl-5-(trifluoromethyl)phenyl)urea;
3-(3-chloro-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(2-(2-methoxyethyl)-1-methyl-1H-imidazo[1,2-b]pyrazole-7-carbonyl)-2-azaspiro[3.3]heptan-6-yl)-1-methyl-3-(5-(trifluoromethyl)pyridin-3-yl)urea;
3-(4-bromopyridin-2-yl)-1-methyl-1-(2-(pyrazolo[5,1-b]thiazole-7-carbonyl)-2-azaspiro[3.3]heptan-6-yl)urea;
1-(2-(6-(1-(2-methoxyethyl)-1H-pyrazol-4-yl) pyrazolo[1,5-a]pyridine-3-carbonyl)-2-azaspiro[3.3]heptan-6-yl)-1-methyl-3-(5-(trifluoromethyl)pyridin-3-yl)urea;
1-methyl-1-(2-(pyrazolo[5,1-b]thiazole-7-carbonyl)-2-azaspiro[3.3]heptan-6-yl)-3-(5-(trifluoromethyl)pyridazin-3-yl)urea;
1-methyl-1-(2-(6-(2-methyloxazol-5-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(6-(pyridin-3-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(6-(1-methyl-1H-pyrazol-4-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(1-methyl-1H-imidazo[1,2-b]pyrazole-7-carbonyl)-2-azaspiro[3.3]heptan-6-yl)-3-(2-methyl-5-(trifluoromethyl)phenyl)urea;
1-(2-(6-(3,4-dimethoxyphenyl) pyrazolo[1,5-a]pyridine-3-carbonyl)-2-azaspiro[3.3]heptan-6-yl)-1-methyl-3-(5-(trifluoromethyl)pyridin-3-yl)urea;
1-methyl-1-(2-(6-(2-methylthiazol-5-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(pyrazolo[5,1-b]thiazole-7-carbonyl)-2-azaspiro[3.3]heptan-6-yl)-3-(5-(1,1,1-trifluoro-2-methylpropan-2-yl) isoxazol-3-yl)urea;
1-(2-(6-(2-methoxypyridin-4-yl) pyrazolo[1,5-a]pyridine-3-carbonyl)-2-azaspiro[3.3]heptan-6-yl)-1-methyl-3-(5-(trifluoromethyl)pyridin-3-yl)urea;
1-methyl-1-(2-(6-(3-methylisoxazol-5-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(pyrazolo[1,5-a]pyridine-3-carbonyl)-2-azaspiro[3.3]heptan-6-yl)-3-(4-(trifluoromethyl)pyridin-2-yl)urea;
1-methyl-1-(2-(pyrazolo[1,5-a]pyridine-3-carbonyl)-2-azaspiro[3.3]heptan-6-yl)-3-(4-(trifluoromethoxy)pyridin-2-yl)urea;
1-methyl-1-(2-(6-(pyrrolidin-1-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(6-(1-methyl-1H-pyrazol-4-yl) pyrazolo[1,5-a]pyridine-3-carbonyl)-2-azaspiro[3.3]heptan-6-yl)-3-(4-(trifluoromethyl)pyridin-2-yl)urea;
1-(2-(6-(1,2-dimethyl-1H-214-pyrazol-4-yl) pyrazolo[1,5-a]pyridine-3-carbonyl)-2-azaspiro[3.3]heptan-6-yl)-1-methyl-3-(4-(trifluoromethoxy)pyridin-2-yl)urea;
1-methyl-1-(2-(5-(1-methyl-1H-pyrazol-4-yl) pyrazolo[1,5-a]pyridine-3-carbonyl)-2-azaspiro[3.3]heptan-6-yl)-3-(5-(trifluoromethyl)pyridin-3-yl)urea;
1-(2-(5-aminopyrazolo[1,5-a]pyridine-3-carbonyl)-2-azaspiro[3.3]heptan-6-yl)-1-methyl-3-(5-(trifluoromethyl)pyridin-3-yl)urea;
1-methyl-1-(2-(6-(pyrimidin-5-yl) pyrazolo[1,5-a]pyridine-3-carbonyl)-2-azaspiro[3.3]heptan-6-yl)-3-(5-(trifluoromethyl)pyridin-3-yl)urea;
1-methyl-1-(2-(6-(2-methylpyrimidin-5-yl) pyrazolo[1,5-a]pyridine-3-carbonyl)-2-azaspiro[3.3]heptan-6-yl)-3-(5-(trifluoromethyl)pyridin-3-yl)urea;
1-(2-(7H-pyrrolo[2,3-d]pyrimidine-4-carbonyl)-2-azaspiro[3.3]heptan-6-yl)-1-methyl-3-(3-(trifluoromethyl)phenyl)urea;
1-(2-(1H-pyrrolo[2,3-b]pyridine-4-carbonyl)-2-azaspiro[3.3]heptan-6-yl)-1-methyl-3-(3-(trifluoromethyl)phenyl)urea;
1-methyl-1-(2-(6-(pyridin-4-yl) pyrazolo[1,5-a]pyridine-3-carbonyl)-2-azaspiro[3.3]heptan-6-yl)-3-(5-(trifluoromethyl)pyridin-3-yl)urea;
1-(2-(6-methoxypyrazolo[1,5-a]pyridine-3-carbonyl)-2-azaspiro[3.3]heptan-6-yl)-1-methyl-3-(5-(trifluoromethyl)pyridin-3-yl)urea;
1-(2-(5-methoxypyrazolo[1,5-a]pyridine-3-carbonyl)-2-azaspiro[3.3]heptan-6-yl)-1-methyl-3-(5-(trifluoromethyl)pyridin-3-yl)urea;
1-(2-(5-(3,3-difluoropyrrolidin-1-yl) pyrazolo[1,5-a]pyridine-3-carbonyl)-2-azaspiro[3.3]heptan-6-yl)-1-methyl-3-(5-(trifluoromethyl)pyridin-3-yl)urea;
1-(2-(6-(6-methoxypyridin-3-yl) pyrazolo[1,5-a]pyridine-3-carbonyl)-2-azaspiro[3.3]heptan-6-yl)-1-methyl-3-(5-(trifluoromethyl)pyridin-3-yl)urea;
3-(4-iodopyridin-2-yl)-1-methyl-1-(2-(1-methyl-1H-imidazo[1,2-b]pyrazole-7-carbonyl)-2-azaspiro[3.3]heptan-6-yl)urea;
3-(4-(difluoromethyl)pyridin-2-yl)-1-methyl-1-(2-(pyrazolo[5,1-b]thiazole-7-carbonyl)-2-azaspiro[3.3]heptan-6-yl)urea;
3-(6-methoxy-4-(trifluoromethyl)pyridin-2-yl)-1-methyl-1-(2-(1-methyl-1H-imidazo[1,2-b]pyrazole-7-carbonyl)-2-azaspiro[3.3]heptan-6-yl)urea;
3-(3,5-bis(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(6-(1,3-dimethyl-1H-pyrazol-4-yl) pyrazolo[1,5-a]pyridine-3-carbonyl)-2-azaspiro[3.3]heptan-6-yl)-1-methyl-3-(5-(trifluoromethyl)pyridin-3-yl)urea;
1-methyl-1-(2-(pyrazolo[1,5-a]pyridine-3-carbonyl)-2-azaspiro[3.3]heptan-6-yl)-3-(5-(trifluoromethyl)pyridazin-3-yl)urea;
1-(2-(7H-pyrrolo[2,3-d]pyrimidine-4-carbonyl)-2-azaspiro[3.3]heptan-6-yl)-3-(2-methoxy-5-(trifluoromethyl)phenyl)-1-methylurea;
1-(2-(6-(1,5-dimethyl-1H-pyrazol-4-yl) pyrazolo[1,5-a]pyridine-3-carbonyl)-2-azaspiro[3.3]heptan-6-yl)-1-methyl-3-(5-(trifluoromethyl)pyridin-3-yl)urea;
1-(2-methoxy-5-(trifluoromethyl)pyridin-3-yl)-3-(2-(pyrazolo[5,1-b]thiazole-7-carbonyl)-2-azaspiro[3.3]heptan-6-yl)urea;
1-methyl-1-(2-(thieno[3,2-d]pyrimidine-4-carbonyl)-2-azaspiro[3.3]heptan-6-yl)-3-(4-(trifluoromethyl)pyridin-2-yl)urea;
3-(2-ethyl-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
3-(3-fluoro-4-methyl-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(5-(perfluoroethyl)pyridin-3-yl)urea;
3-(6-chloro-4-(trifluoromethyl)pyridin-2-yl)-1-methyl-1-(2-(1-methyl-1H-imidazo[1,2-b]pyrazole-7-carbonyl)-2-azaspiro[3.3]heptan-6-yl)urea;
1-(2-(7H-pyrrolo[2,3-d]pyrimidine-4-carbonyl)-2-azaspiro[3.3]heptan-6-yl)-1-methyl-3-(3-methyl-5-(trifluoromethyl)phenyl)urea;
1-methyl-1-(2-(1-methyl-1H-imidazo[1,2-b]pyrazole-7-carbonyl)-2-azaspiro[3.3]heptan-6-yl)-3-(3-(1,1,1-trifluoro-2-methylpropan-2-yl) isoxazol-5-yl)urea;
2-(1-methyl-1H-imidazo[1,2-b]pyrazole-7-carbonyl)-2-azaspiro[3.3]heptan-6-yl(2-methyl-5-(trifluoromethyl)phenyl)carbamate;
2-(1-methyl-1H-imidazo[1,2-b]pyrazole-7-carbonyl)-2-azaspiro[3.3]heptan-6-yl(5-(1,1,1-trifluoro-2-methylpropan-2-yl) isoxazol-3-yl)carbamate;
2-(pyrazolo[5,1-b]thiazole-7-carbonyl)-2-azaspiro[3.3]heptan-6-yl(4-(trifluoromethoxy)pyridin-2-yl)carbamate;
2-(pyrazolo[5,1-b]thiazole-7-carbonyl)-2-azaspiro[3.3]heptan-6-yl(5-(trifluoromethyl)pyridin-3-yl)carbamate;
2-(2-(1-methyl-1H-imidazo[1,2-b]pyrazole-7-carbonyl)-2-azaspiro[3.3]heptan-6-yl)-N-(5-(trifluoromethyl)pyridin-3-yl) acetamide;
2-(2-(pyrazolo[5,1-b]thiazole-7-carbonyl)-2-azaspiro[3.3]heptan-6-yl)-N-(5-(trifluoromethoxy)pyridin-3-yl) acetamide;
N-(2-fluoro-5-(trifluoromethoxy)phenyl)-2-(2-(1-methyl-1H-imidazo[1,2-b]pyrazole-7-carbonyl)-2-azaspiro[3.3]heptan-6-yl) acetamide;
2-(2-(1-methyl-1H-imidazo[1,2-b]pyrazole-7-carbonyl)-2-azaspiro[3.3]heptan-6-yl)-N-(4-(trifluoromethoxy)pyridin-2-yl) acetamide;
2-(2-(1-methyl-1H-imidazo[1,2-b]pyrazole-7-carbonyl)-2-azaspiro[3.3]heptan-6-yl)-N-(2-methyl-5-(trifluoromethoxy)phenyl)acetamide;
2-(2-(1-methyl-1H-imidazo[1,2-b]pyrazole-7-carbonyl)-2-azaspiro[3.3]heptan-6-yl)-N-(4-(trifluoromethyl)pyridin-2-yl) acetamide;
1-methyl-3-(2-methyl-5-(trifluoromethoxy)phenyl)-1-(2-(thieno[3,2-d]pyrimidine-4-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(3-methyl-5-(trifluoromethoxy)phenyl)urea);
1-(2-ethyl-5-(trifluoromethyl)phenyl)-3-(2-(1-methyl-1H-imidazo[1,2-b]pyrazole-7-carbonyl)-2-azaspiro[3.3]heptan-6-yl)urea;
3-(3-fluoro-2-methyl-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-(2-(7H-pyrrolo[2,3-d]pyrimidine-4-carbonyl)-2-azaspiro[3.3]heptan-6-yl)-3-(3-cyano-5-(trifluoromethyl)phenyl)-1-methylurea;
1-(2-(7H-pyrrolo[2,3-d]pyrimidine-4-carbonyl)-2-azaspiro[3.3]heptan-6-yl)-3-(2-chloro-5-(trifluoromethyl)phenyl)-1-methylurea;
1-(2-(7H-pyrrolo[2,3-d]pyrimidine-4-carbonyl)-2-azaspiro[3.3]heptan-6-yl)-3-(3-chloro-5-(trifluoromethyl)phenyl)-1-methylurea;
3-(3-methoxy-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
3-(2-ethoxy-5-(trifluoromethyl)phenyl)-1-methyl-1-(2-(1-methyl-1H-imidazo[1,2-b]pyrazole-7-carbonyl)-2-azaspiro[3.3]heptan-6-yl)urea;
1-methyl-3-(2-methyl-6-(trifluoromethyl)pyrimidin-4-yl)-1-(2-(pyrazolo[5,1-b]thiazole-7-carbonyl)-2-azaspiro[3.3]heptan-6-yl)urea;
1-methyl-1-(2-(1-methyl-1H-imidazo[1,2-b]pyrazole-7-carbonyl)-2-azaspiro[3.3]heptan-6-yl)-3-(2-methyl-6-(trifluoromethyl)pyrimidin-4-yl)urea;
1-methyl-1-(2-(1-methyl-1H-imidazo[1,2-b]pyrazole-7-carbonyl)-2-azaspiro[3.3]heptan-6-yl)-3-(4-(trifluoromethyl)thiophen-2-yl)urea;
2-(pyrazolo[5,1-b]thiazole-7-carbonyl)-2-azaspiro[3.3]heptan-6-yl(6-(trifluoromethyl)pyrimidin-4-yl)carbamate;
2-(pyrazolo[5,1-b]thiazole-7-carbonyl)-2-azaspiro[3.3]heptan-6-yl(5-(1,1,1-trifluoro-2-methylpropan-2-yl) isoxazol-3-yl)carbamate;
2-(1-methyl-1H-imidazo[1,2-b]pyrazole-7-carbonyl)-2-azaspiro[3.3]heptan-6-yl(4-(trifluoromethoxy)pyridin-2-yl)carbamate;
2-(1-methyl-1H-imidazo[1,2-b]pyrazole-7-carbonyl)-2-azaspiro[3.3]heptan-6-yl(2-methyl-5-(trifluoromethoxy)phenyl)carbamate;
2-(1-methyl-1H-imidazo[1,2-b]pyrazole-7-carbonyl)-2-azaspiro[3.3]heptan-6-yl(4-(trifluoromethyl)pyridin-2-yl)carbamate;
2-(1-methyl-1H-imidazo[1,2-b]pyrazole-7-carbonyl)-2-azaspiro[3.3]heptan-6-yl(6-(trifluoromethyl)pyrimidin-4-yl)carbamate;
1-(2-ethoxy-5-(trifluoromethyl)phenyl)-3-(2-(1-methyl-1H-imidazo[1,2-b]pyrazole-7-carbonyl)-2-azaspiro[3.3]heptan-6-yl)urea;
2-(1-methyl-1H-imidazo[1,2-b]pyrazole-7-carbonyl)-2-azaspiro[3.3]heptan-6-yl(5-(trifluoromethoxy)pyridin-3-yl)carbamate;
1-methyl-1-(2-(1-methyl-1H-imidazo[1,2-b]pyrazole-7-carbonyl)-2-azaspiro[3.3]heptan-6-yl)-3-(6-methyl-4-(trifluoromethyl)pyridin-2-yl)urea;
2-(2-(1-(methyl-d3)-1H-imidazo[1,2-b]pyrazole-7-carbonyl)-2-azaspiro[3.3]heptan-6-yl)-N-(4-(trifluoromethoxy)pyridin-2-yl) acetamide;
2-(2-(1-(difluoromethyl)-1H-imidazo[1,2-b]pyrazole-7-carbonyl)-2-azaspiro[3.3]heptan-6-yl)-N-(4-(trifluoromethoxy)pyridin-2-yl) acetamide;
1-methyl-1-(2-(1-methyl-1H-imidazo[1,2-b]pyrazole-7-carbonyl)-2-azaspiro[3.3]heptan-6-yl)-3-(5-methyl-4-(trifluoromethyl)pyridin-2-yl)urea;
1-(2-(1H-pyrazolo[3,4-b]pyridin-5-yl)-2-azaspiro[3.3]heptan-6-yl)-3-(5-(trifluoromethyl)pyridin-3-yl)urea;
1-methyl-1-(2-(1-methyl-1H-imidazo[1,2-b]pyrazole-7-carbonyl)-2-azaspiro[3.3]heptan-6-yl)-3-(4-(perfluoroethyl)pyridin-2-yl)urea;
1-(2-(1H-pyrazolo[3,4-b]pyridin-5-yl)-2-azaspiro[3.3]heptan-6-yl)-3-(5-(trifluoromethyl)pyridazin-3-yl)urea;
1-(2-(1H-pyrazolo[3,4-b]pyridin-5-yl)-2-azaspiro[3.3]heptan-6-yl)-3-(2-methyl-5-(trifluoromethoxy)phenyl)urea;
3-(6-bromo-4-(trifluoromethyl)pyridin-2-yl)-1-methyl-1-(2-(1-methyl-1H-imidazo[1,2-b]pyrazole-7-carbonyl)-2-azaspiro[3.3]heptan-6-yl)urea;
1-(2-ethyl-5-(trifluoromethyl)pyridin-3-yl)-3-(2-(1-methyl-1H-imidazo[1,2-b]pyrazole-7-carbonyl)-2-azaspiro[3.3]heptan-6-yl)urea;
3-(2-ethyl-5-(trifluoromethyl)pyridin-3-yl)-1-methyl-1-(2-(1-methyl-1H-imidazo[1,2-b]pyrazole-7-carbonyl)-2-azaspiro[3.3]heptan-6-yl)urea;
2-(1-methyl-1H-imidazo[1,2-b]pyrazole-7-carbonyl)-2-azaspiro[3.3]heptan-6-yl(5-(trifluoromethyl)pyridazin-3-yl)carbamate; and
1-(2-(1H-pyrazolo[3,4-b]pyridin-5-yl)-2-azaspiro[3.3]heptan-6-yl)-3-(3-(hydroxymethyl)-2-methoxy-5-(trifluoromethyl)phenyl)urea.

11. A pharmaceutical composition comprising the compound, or stereoisomer, tautomer, solvate or pharmaceutically acceptable salt thereof, according to claim 1, in admixture with at least one of more pharmaceutically acceptable carrier and/or excipient.

12. The pharmaceutical composition according to claim 11 formulated for administration by inhalation.

13. The pharmaceutical composition according to claim 11 formulated for oral administration.

14. (canceled)

15. A method of treating a disease disorder or condition associated with dysregulation of DDR comprising administering to a subject in need thereof a compound of formula (I) 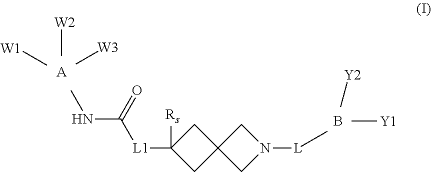
wherein
A is a ring selected from the group consisting of: 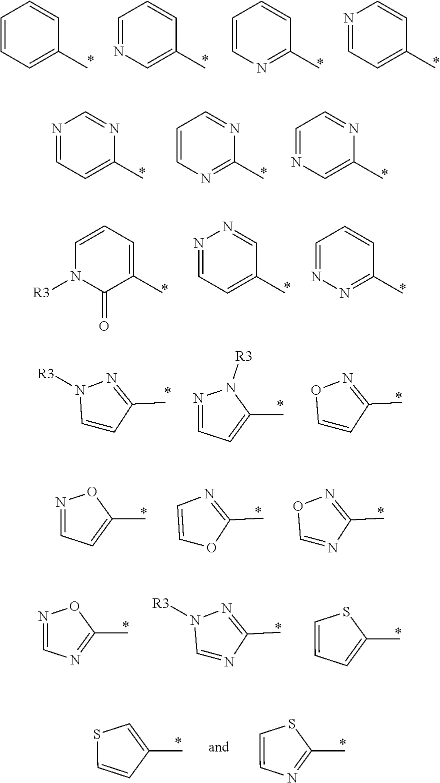
wherein [symbol] indicates a direct bond to NH;
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms;
L is selected from the group consisting of CO and CH₂ or is absent;
L1 is selected from the group consisting of NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇)cycloalkyl-(C₁-C₄)alkyl and deuterated (C₁-C₄)alkyl;
R_{S} is selected from the group consisting of hydrogen and methyl, when L1 is NR;
R_{S} is hydrogen, when L1 is CH₂ or O;
B is a mono- or bi-cyclic heteroaryl ring or bi-cyclic semisaturated heteroaryl ring;
Y1 and Y2 are substituents of ring B independently selected from the group consisting of hydrogen, (C₁-C₄)alkyl, deuterated (C₁-C₄)alkyl, (C₁-C₄)haloalkyl, (C₁-C₄)alkoxy, (C₁-C₄)haloalkoxy, (C₁-C₄)hydroxyalkyl, halogen, cyano, SF₅, (C₁-C₄) cyanoalkyl, (C₁-C₄)alkyl-sulfonyl, (C₁-C₄)haloalkyl-sulfonyl, CONR1R2, NHCOR1, NR1R2, NR1R2-(C₁-C₄)alkyl, heterocycloalkyl optionally substituted with 1 to 3 halogens, heterocycloalkyl-NH—(C₁-C₄)alkyl, (C₃-C₇)cycloalkyl, (C₁-C₄)alkyl-heterocycloalkyl, (C₁-C₄)alkyl-(C₃-C₇)cycloalkyl, heterocycloalkyl-(C₁-C₄)alkyl, (C₃-C₇)cycloalkyl-(C₁-C₄)alkyl, heterocycloalkyl-(C₁-C₄)alkoxy, (C₃-C₇)cycloalkyl-(C₁-C₄)alkoxy, heterocycloalkyl-oxy, (C₃-C₇)cycloalkyl-oxy, phenyl, (C₁-C₄)alkoxy substituted phenyl, (C₁-C₄)alkyl-thio-(C₁-C₄)alkyl, (C₁-C₄)alkyl-sulfonyl-(C₁-C₄)alkyl, (C₁-C₄)alkoxy-(C₁-C₄)alkyl, (C₁-C₄)haloalkyl-cycloalkyl, (C₁-C₄)alkyl-heterocycloalkyl-carbonyl and monocyclic heteroaryl, optionally substituted with 1 to 3 groups selected from the group consisting of (C₁-C₄)alkyl, (C₁-C₄)alkoxy and (C₁-C₄)alkoxy-(C₁-C₄)alkyl;
R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl;
R3 is selected from the group consisting of (C₁-C₄)alkyl, (C₁-C₄)haloalkyl, (C₁-C₄)alkyl-phenyl and monocyclic heteroaryl;
or a stereoisomer, tautomer, solvate or pharmaceutically acceptable salt thereof.

16. The method according to claim 15, wherein, when A ring is phenyl and L1 is NH,
W1, W2 and W3 are independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms.

17. The method according to claim 15, wherein the disease, disorder or condition associated with dysregulation of DDR comprises fibrosis and/or a disease, disorder or condition that involves fibrosis.

18. The method according to claim 17, wherein the fibrosis comprises at least one selected from the group consisting of pulmonary fibrosis, idiopathic pulmonary fibrosis (IPF), hepatic fibrosis, progressive pulmonary fibrosis, sarcoidosis, familiar pulmonary fibrosis, chronic hypersensitivity pneumonitis (CHP), kidney fibrosis, renal fibrosis, ocular fibrosis, cardiac fibrosis, arterial fibrosis and systemic sclerosis.

19. The method according to claim 18, wherein the fibrosis comprises idiopathic pulmonary fibrosis (IPF).

20. The method according to claim 18, wherein the fibrosis comprises kidney fibrosis.

21. A compound of formula IV 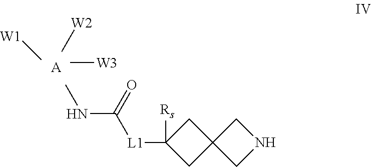
wherein
A is a ring selected from the group consisting of: 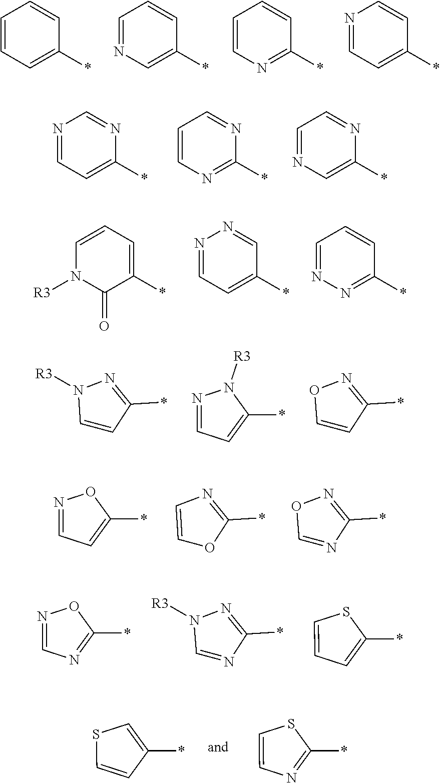
wherein [symbol] indicates a direct bond to NH;
W1, W2 and W3 are substituents of ring A independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, halogen, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms;
L1 is selected from the group consisting of NR, CH₂ and O, wherein R is selected from the group consisting of hydrogen, (C₁-C₄)alkyl, (C₁-C₄)hydroxyalkyl, (C₁-C₄)haloalkyl, (C₃-C₇)cycloalkyl, (C₃-C₇)cycloalkyl-(C₁-C₄)alkyl and deuterated (C₁-C₄)alkyl;
Rs is selected from the group consisting of hydrogen and methyl when L1 is NR;
R_{S} is hydrogen when L1 is CH₂ or O;
R1 and R2 are independently selected from the group consisting of hydrogen, (C₁-C₄)alkyl, (C₁-C₄)hydroxyalkyl, (C₁-C₄)alkoxy-(C₁-C₄)alkyl, (C₁-C₄)alkylamino-(C₁-C₄)alkyl, di-(C₁-C₄)alkylamino-(C₁-C₄)alkyl, optionally substituted (C₃-C₇)cycloalkyl, optionally substituted heterocycloalkyl and optionally substituted heterocycloalkyl-(C₁-C₄)alkoxy, wherein optional substituents are from 1 to 3 and are selected from the group consisting of (C₁-C₄)alkyl, (C₁-C₄)alkoxy, (C₁-C₄)haloalkyl, (C₁-C₄)haloalkoxy and carbamoyl; and
R3 is selected from the group consisting of (C₁-C₄)alkyl, (C₁-C₄)haloalkyl, (C₁-C₄)alkyl-phenyl and monocyclic heteroaryl;
or a stereoisomer, tautomer, solvate or pharmaceutically acceptable salt thereof;
wherein the compound of formula IV is not 1-(3,5-dichlorophenyl)-3-(2-azaspiro[3.3]heptan-6-yl)urea;
and wherein, when A ring is phenyl, L1 is NH and W1 and W2 are hydrogen, W3 is not para-OCF₃.

22. The compound, or stereoisomer, tautomer, solvate or pharmaceutically acceptable salt thereof, of claim 21, wherein, when A ring is phenyl and L1 is NH,
W1, W2 and W3 are independently selected from the group consisting of hydrogen, (C₁-C₄)alkyl, (C₁-C₄)haloalkyl, (C₁-C₄)halohydroxyalkyl, (C₁-C₄)alkoxy, (C₁-C₄)alkoxy-(C₁-C₄)alkyl, (C₁-C₄)haloalkoxy, (C₁-C₄)hydroxyalkyl, fluorine, bromine, iodine, cyano, SF₅, NR1R2-(C₁-C₄)alkyl, CONR1R2, NHCOR1, NR1R2, heterocycloalkyl, (C₃-C₇)cycloalkyl, (C₁-C₄)alkyl-heterocycloalkyl, (C₁-C₄)alkyl-heterocycloalkyl-(C₁-C₄)alkyl, (C₁-C₄)haloalkyl-cycloalkyl, (C₁-C₄)alkyl-(C₃-C₇)cycloalkyl, heterocycloalkyl-(C₁-C₄)alkyl, heterocycloalkyl-NH—(C₁-C₄)alkyl, (C₃-C₇)cycloalkyl-(C₁-C₄)alkyl, heterocycloalkyl-(C₁-C₄)alkoxy, (C₁-C₄)alkyl-CO-heterocycloalkyl-oxy, (C₃-C₇)cycloalkyl-(C₁-C₄)alkoxy, heterocycloalkyl-oxy, (C₃-C₇)cycloalkyl-oxy, (C₁-C₄)alkyl-heterocycloalkyl-carbonyl, monocyclic (C₁-C₄)alkyl-heteroaryl, (C₁-C₄)alkyl-sulfonyl, (C₁-C₄)haloalkyl-sulfonyl, (C₁-C₄)haloalkyl-sulfonylamino, (C₁-C₄)haloalkyl-sulfinyl, (C₁-C₄)haloalkyl-thio, (C₁-C₄)alkyl-thio-(C₁-C₄)alkyl, (C₁-C₄)alkyl-sulfonyl-(C₁-C₄)alkyl and (C₁-C₄)alkyl-sulfinyl-(C₁-C₄)alkyl; or, wherein W1 and W2 are in adjacent positions on ring A, W1 and W2 form a (C₅-C₆)cycloalkyl or a 5- or 6-membered heterocycloalkyl, wherein the (C₅-C₆)cycloalkyl or the 5- or 6-membered heterocycloalkyl are optionally substituted with 1 to 3 halogen atoms.

23. (canceled)

24. A process for the preparation of the compound, or stereoisomer, tautomer solvate or pharmaceutically acceptable salt thereof, according to claim 1, comprising:
a) reacting an intermediate compound IV 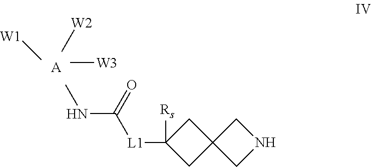
with either a carboxylic acid XI 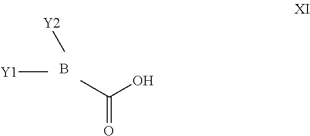
or an aldehyde XII 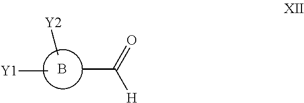
or an aryl halide XIII 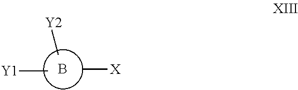
to obtain the compound, or stereoisomer, tautomer, solvate or pharmaceutically acceptable salt thereof, according to claim 1,
wherein A, W1, W2, W3, L1, R_{S}, B, Y1 and Y2 are as defined in claim 1 and X is halogen.
